(19) United States
(12) Patent Application Publication
(10) Pub. No.: US 2026/0200919 A1
(43) Pub. Date: Jul. 16, 2026

(54) SUBSTITUTED AMINOPYRIDINE COMPOUNDS AND METHODS OF TREATING DISEASE USING SAME

(71) Applicant: Atavistik Bio, Inc., Cambridge, MA (US)

(72) Inventors: Maria Jesus Blanco-Pillado, Arlington, MA (US); Aaron Robert Coffin, Everett, MA (US)

(21) Appl. No.: 19/561,275

(22) Filed: Mar. 9, 2026

Related U.S. Application Data
(63) Continuation of application No. PCT/US2024/046748, filed on Sep. 13, 2024.
(60) Provisional application No. 63/538,656, filed on Sep. 15, 2023.

(51) Int. Cl. C07D 471/04; A61K 31/4439; A61K 31/444; A61K 31/4545; C07D 401/04; C07D 401/14

(52) U.S. Cl. CPC C07D 471/04; A61K 31/4439; A61K 31/444; A61K 31/4545; C07D 401/04; C07D 401/14

(57) ABSTRACT

The invention provides aminopyridinyl imidazo[4,5-b]pyridine and related compounds, pharmaceutical compositions, and their use in treating disease, such as cancer.

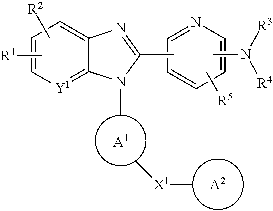

### CROSS-REFERENCE TO RELATED APPLICATION

This application claims priority to and the benefit of U.S. Provisional Patent Application No. 63/538,656, filed Sep. 15, 2023, the content of which is hereby incorporated by reference in its entirety.

### BRIEF SUMMARY

The invention provides aminopyridinyl imidazo[4,5-b]pyridine and related compounds, pharmaceutical compositions, and their use in treating disease, such as cancer.

### BACKGROUND

Cancer continues to be a significant health problem despite the substantial research efforts and scientific advances reported in the literature for treating this disease. Solid tumors, such as prostate cancer, colon, rectum, skin cancer, breast cancer, and lung cancer remain highly prevalent among the world population. Existing therapies for treating cancer include localized therapies, such as surgery, radiation therapy, cryotherapy, and systemic therapies (e.g., chemotherapy, hormonal therapy, immune therapy, and targeted therapy) used alone or in combination. Support therapies are also used in some contexts, where supportive therapies are additional treatments that do not directly treat cancer but are used to reduce side effects and address patient quality of life. However, current treatment options for cancer are not effective for all patients and/or can have substantial adverse side effects. New therapies are needed to address this unmet need in cancer therapy.

The AKT protein family, which members are also named as protein kinase B (PKB), are important mediators of growth factor induced cellular signaling. See, for example, Manning and Toker, Cell 169, Apr. 20, 2017. The AKT protein family belongs to the larger serine/threonine kinase family regulated by the phosphoinositide 3-kinase (PI3K) pathway—a critical signal transduction system linking oncogenes and multiple receptor classes to many essential cellular functions. Class I PI3K phosphorylates the 3′ hydroxyl of the inositol bead group of phosphoinositides (PtdIns), resulting in the production of the lipid second messengers PtdIns-3,4-P₂ (PIP2) and PtdIns3,4,5-P3 (PIP3). PIP3 recruits AKT to the plasma membrane where it is activated and triggers a signaling cascade impinging upon pathways important for cancer cell growth.

In humans, there are three AKT isoforms encoded by three separate genes: AKT1, AKT2 and AKT3. These genes encode for serine/threonine kinases that are activated by growth factors in a phosphoinositide 3-kinase (PI3K) dependent manner. AKT1 regulates cell growth and survival and is expressed in several tissues. AKT2 is an important signaling molecule in the insulin signaling pathway and is required to induce glucose transport in certain tissues. The role of AKT3 is less known, and it appears to be predominantly expressed in the brain.

Among the three AKT isoforms, AKT1 is most frequently hyperactivated in tumors. The hyperactivation of AKT1 in tumors underscores the important role AKT1 plays in tumorigenesis and cancer cell proliferation and survival (Bhattarai et al. Nature Communications volume 13, Article number: 2111, 2022). AKT1 signaling promotes tumor cell growth by modulating multiple different effectors. For example, AKT1 regulates protein synthesis via the mTORC1 pathway, cellular metabolism via GSK3-beta pathway and multiple metabolic enzymes, and cell profileration and survival via the FOXO family of transcription factors. See, for example, Hoxhaj and Manning, Nat Rev Cancer. 2020 February; 20 (2): 74-88.

The most common mutation in AKT1 is a point mutation converting a glutamic acid of residue 17 to a lysine (E17K). The E17K mutation [Carpten et al. Nature 448, 2007, 439-444] in the Pleckstrin Homology Domain (PHD) of AKT1 leads to constitutive association with the plasma membrane via ectopic binding to PIP2 and increased affinity for PIP3. Consequently, this deregulated recruitment of AKT1 to the plasma membrane causes constitutive activation of AKT1 signaling and has been shown to be a recurrent mutation in breast, endometrial, and other cancers.

The oncogenic properties of the E17K single point mutation in AKT1 make it a target for inhibition for anti-cancer agents. In addition to cancer, somatic mutation of AKT1 to the E17K variant during embryonic development leads to the overgrowth disease Proteus Syndrome (N Engl J Med. 2011 Aug. 18; 365 (7): 611-619). Proteus syndrome is characterized by the overgrowth of skin, connective tissue, brain, and other tissues. Patients with this disease also have an increased risk of premature death due to vascular deformities.

New compounds that inhibit AKT1 are needed and would provide a medical benefit to patients suffering from disorders associated with AKT1 activity. The present invention addresses the foregoing needs and provides other related advantages.

### SUMMARY

The invention provides aminopyridinyl imidazo[4,5-b]pyridine and related compounds, pharmaceutical compositions, and their use in treating disease, such as cancer. In particular, one aspect of the invention provides a collection of aminopyridinyl imidazo[4,5-b]pyridine and related compounds, such as a compound represented by Formula I:

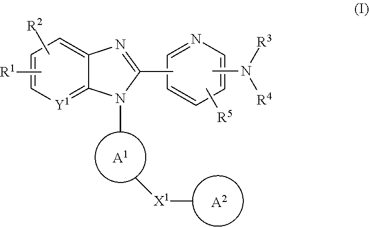

or a pharmaceutically acceptable salt thereof, where the variables are as defined in the detailed description. Further description of additional collections of heterosubstituted acetyl imidazo[5,1-d]tetrazinone compounds are described in the detailed description. The compounds may be part of a pharmaceutical composition comprising a pharmaceutically acceptable carrier.

Another aspect of the invention provides a method of treating a disease or disorder associated with aberrant AKT1 signaling. The method comprises administering to a subject in need thereof a therapeutically effective amount of a compound described herein, such as a compound of Formula I, to treat the disease or disorder, as further described in the detailed description.

Another aspect of the invention provides a method of treating cancer. The method comprises administering to a subject in need thereof a therapeutically effective amount of a compound described herein, such as a compound of Formula I, to treat the cancer, as further described in the detailed description. In certain embodiments, the cancer has a AKT1 mutation.

A method of treating a disease or disorder associated with active PI3K signaling. The method comprises administering to a subject in need thereof a therapeutically effective amount of a compound described herein, such as a compound of Formula I, to treat the disease or disorder.

Another aspect of the invention provides a method inhibiting AKT1 activity. The method comprises contacting an AKT1 with an effective amount of a compound described herein, such as a compound of Formula I, to thereby inhibit the AKT1 activity, as further described in the detailed description.

### DETAILED DESCRIPTION

The invention provides aminopyridinyl imidazo[4,5-b]pyridine and related compounds, pharmaceutical compositions, and their use in treating disease, such as cancer. The practice of the present invention employs, unless otherwise indicated, conventional techniques of organic chemistry, pharmacology, molecular biology (including recombinant techniques), cell biology, biochemistry, and immunology. Such techniques are explained in the literature, such as in “Comprehensive Organic Synthesis” (B. M. Trost & I. Fleming, eds., 1991-1992); “Handbook of experimental immunology” (D. M. Weir & C. C. Blackwell, eds.); “Current protocols in molecular biology” (F. M. Ausubel et al., eds., 1987, and periodic updates); and “Current protocols in immunology” (J. E. Coligan et al., eds., 1991), each of which is herein incorporated by reference in its entirety.

Various aspects of the invention are set forth below in sections; however, aspects of the invention described in one particular section are not to be limited to any particular section. Further, when a variable is not accompanied by a definition, the previous definition of the variable controls.

### Definitions

Compounds of the present invention include those described generally herein, and are further illustrated by the classes, subclasses, and species disclosed herein. As used herein, the following definitions shall apply unless otherwise indicated. These definitions apply regardless of whether a term is used by itself or in combination with other terms, unless otherwise indicated. Hence, the definition of “alkyl” applies to “alkyl” as well as the “alkyl” portions of “—O-alkyl” etc. For purposes of this invention, the chemical elements are identified in accordance with the Periodic Table of the Elements, CAS version, Handbook of Chemistry and Physics, 75^{th} Ed. Additionally, general principles of organic chemistry are described in “Organic Chemistry”, Thomas Sorrell, University Science Books, Sausalito: 1999, and “March's Advanced Organic Chemistry”, 5^{th} Ed., Ed.: Smith, M. B. and March, J., John Wiley & Sons, New York: 2001, the entire contents of which are hereby incorporated by reference.

The term “aliphatic” or “aliphatic group”, as used herein, means a straight-chain (i.e., unbranched) or branched, substituted or unsubstituted hydrocarbon chain that is completely saturated or that contains one or more units of unsaturation, or a monocyclic hydrocarbon or bicyclic hydrocarbon that is completely saturated or that contains one or more units of unsaturation, but which is not aromatic (also referred to herein as “cycloaliphatic”), that has a single point of attachment to the rest of the molecule. Unless otherwise specified, aliphatic groups contain 1-6 aliphatic carbon atoms. In certain embodiments, aliphatic groups contain 1-5 aliphatic carbon atoms. In other embodiments, aliphatic groups contain 1-4 aliphatic carbon atoms. In still other embodiments, aliphatic groups contain 1-3 aliphatic carbon atoms, and in yet other embodiments, aliphatic groups contain 1-2 aliphatic carbon atoms. In certain embodiments, “cycloaliphatic” refers to a monocyclic C₃-C₆ hydrocarbon that is completely saturated or that contains one or more units of unsaturation, but which is not aromatic, that has a single point of attachment to the rest of the molecule. Suitable aliphatic groups include, but are not limited to, linear or branched, substituted or unsubstituted alkyl, alkenyl, alkynyl groups and hybrids thereof such as (cycloalkyl)alkyl, (cycloalkenyl)alkyl or (cycloalkyl)alkenyl.

As used herein, the term “bicyclic ring” or “bicyclic ring system” refers to any bicyclic ring system, i.e., carbocyclic or heterocyclic, saturated or having one or more units of unsaturation, having one or more atoms in common between the two rings of the ring system. Thus, the term includes any permissible ring fusion, such as ortho-fused or spirocyclic. As used herein, the term “heterobicyclic” is a subset of “bicyclic” that requires that one or more beteroatoms are present in one or both rings of the bicycle. Such heteroatoms may be present at ring junctions and are optionally substituted, and may be selected from nitrogen (including N-oxides), oxygen, sulfur (including oxidized forms such as sulfones and sulfonates), phosphorus (including oxidized forms such as phosphates), boron, etc. In certain embodiments, a bicyclic group has 7-12 ring members and 0-4 heteroatoms independently selected from nitrogen, oxygen, or sulfur. As used herein, the term “bridged bicyclic” refers to any bicyclic ring system, i.e., carbocyclic or heterocyclic, saturated or partially unsaturated, having at least one bridge. As defined by IUPAC, a “bridge” is an unbranched chain of atoms or an atom or a valence bond connecting two bridgeheads, where a “bridgehead” is any skeletal atom of the ring system which is bonded to three or more skeletal atoms (excluding hydrogen). In certain embodiments, a bridged bicyclic group has 7-12 ring members and 0-4 heteroatoms independently selected from nitrogen, oxygen, or sulfur. Such bridged bicyclic groups are well known in the art and include those groups set forth below where each group is attached to the rest of the molecule at any substitutable carbon or nitrogen atom. Unless otherwise specified, a bridged bicyclic group is optionally substituted with one or more substituents as set forth for aliphatic groups. Additionally or alternatively, any substitutable nitrogen of a bridged bicyclic group is optionally substituted. Exemplary bicyclic rings include:

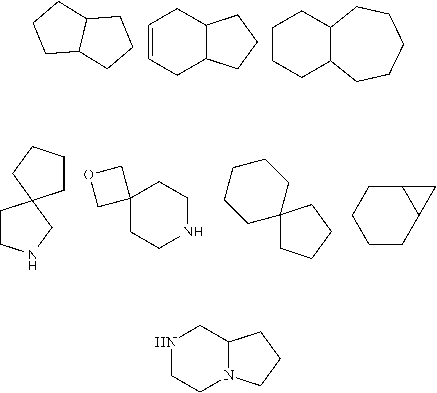

Exemplary bridged bicyclics include:

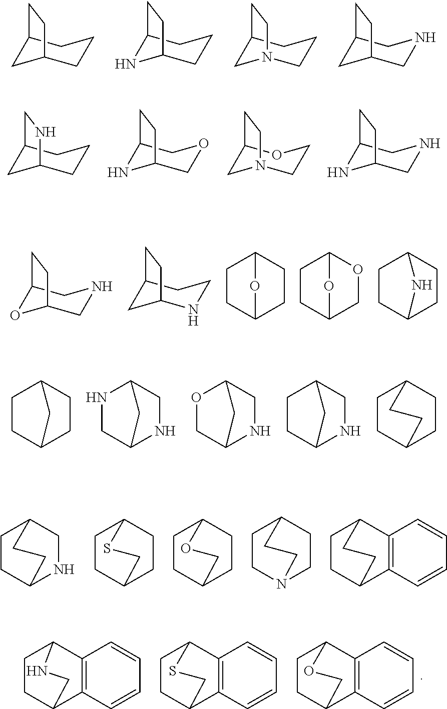

The term “lower alkyl” refers to a C₁₋₄ straight or branched alkyl group. Exemplary lower alkyl groups are methyl, ethyl, propyl, isopropyl, butyl, isobutyl, and tert-butyl.

The term “lower haloalkyl” refers to a C₁₋₄ straight or branched alkyl group that is substituted with one or more halogen atoms.

The term “heteroatom” means one or more of oxygen, sulfur, nitrogen, phosphorus, or silicon (including, any oxidized form of nitrogen, sulfur, phosphorus, or silicon; the quaternized form of any basic nitrogen or; a substitutable nitrogen of a heterocyclic ring, for example N (as in 3,4-dihydro-2H-pyrrolyl), NH (as in pyrrolidinyl) or NR⁺ (as in N-substituted pyrrolidinyl)).

The term “unsaturated,” as used herein, means that a moiety has one or more units of unsaturation.

As used herein, the term “bivalent C₁₋₈ (or C₁₋₆) saturated or unsaturated, straight or branched, hydrocarbon chain”, refers to bivalent alkylene, alkenylene, and alkynylene chains that are straight or branched as defined herein.

The term “alkylene” refers to a bivalent alkyl group. An “alkylene chain” is a polymethylene group, i.e., —(CH₂)ₙ—, wherein n is a positive integer, preferably from 1 to 6, from 1 to 4, from 1 to 3, from 1 to 2, or from 2 to 3. A substituted alkylene chain is a polymethylene group in which one or more methylene hydrogen atoms are replaced with a substituent. Suitable substituents include those described below for a substituted aliphatic group.

The term “—(C₀ alkylene)-” refers to a bond. Accordingly, the term “—(C₀₋₃ alkylene)-” encompasses a bond (i.e., C₀) and a —(C₁₋₃ alkylene)-group.

The term “alkenylene” refers to a bivalent alkenyl group. A substituted alkenylene chain is a polymethylene group containing at least one double bond in which one or more hydrogen atoms are replaced with a substituent. Suitable substituents include those described below for a substituted aliphatic group.

The terms “halogen” and “halo” are used interchangeably and mean F, Cl, Br, or I.

The term “aryl” used alone or as part of a larger moiety as in “aralkyl,” “aralkoxy.” or “aryloxyalkyl,” refers to monocyclic or bicyclic ring systems having a total of five to fourteen ring members, wherein at least one ring in the system is aromatic and wherein each ring in the system contains 3 to 7 ring members. The term “aryl” may be used interchangeably with the term “aryl ring.” In certain embodiments of the present invention, “aryl” refers to an aromatic ring system which includes, but not limited to, phenyl, biphenyl, naphthyl, anthracyl and the like, which may bear one or more substituents. Also included within the scope of the term “aryl,” as it is used herein, is a group in which an aromatic ring is fused to one or more non-aromatic rings, such as indanyl, phthalimidyl, naphthimidyl, phenanthridinyl, or tetrahydronaphthyl, and the like. The term “phenylene” refers to a multivalent phenyl group having the appropriate number of open valences to account for groups attached to it. For example, “phenylene” is a bivalent phenyl group when it has two groups attached to it

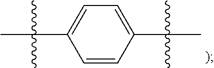 [SMILES: *c1ccc(*)cc1]

“phenylene” is a trivalent phenyl group when it has three groups attached to it

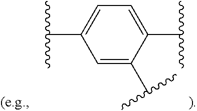 [SMILES: *c1ccc(*)c(*)c1]

The term “arylene” refers to a bivalent aryl group.

The terms “heteroaryl” and “heteroar-,” used alone or as part of a larger moiety, e.g., “heteroaralkyl,” or “heteroaralkoxy,” refer to groups having 5 to 10 ring atoms, preferably 5, 6, or 9 ring atoms; having 6, 10, or 14 π electrons shared in a cyclic array; and having, in addition to carbon atoms, from one to five heteroatoms. The term “heteroatom” refers to nitrogen, oxygen, or sulfur, and includes any oxidized form of nitrogen or sulfur, and any quaternized form of a basic nitrogen. Heteroaryl groups include, without limitation, thienyl, furanyl, pyrrolyl, imidazolyl, pyrazolyl, triazolyl, tetrazolyl, oxazolyl, isoxazolyl, oxadiazolyl, thiazolyl, isothiazolyl, thiadiazolyl, pyridyl, pyridazinyl, pyrimidinyl, pyrazinyl, indolizinyl, purinyl, naphthyridinyl, and pteridinyl. The terms “heteroaryl” and “heteroar-”, as used herein, also include groups in which a heteroaromatic ring is fused to one or more aryl, cycloaliphatic, or heterocyclyl rings, where unless otherwise specified, the radical or point of attachment is on the heteroaromatic ring or on one of the rings to which the heteroaromatic ring is fused. Nonlimiting examples include indolyl, isoindolyl, benzothienyl, benzofuranyl, dibenzofuranyl, indazolyl, benzimidazolyl, benzthiazolyl, quinolyl, isoquinolyl, cinnolinyl, phthalazinyl, quinazolinyl, quinoxalinyl. 4H-quinolizinyl, carbazolyl, acridinyl, phenazinyl, phenothiazinyl, phenoxazinyl, tetrahydroquinolinyl, and tetrahydroisoquinolinyl. A heteroaryl group may be mono- or bicyclic. The term “heteroaryl” may be used interchangeably with the terms “heteroaryl ring,” “heteroaryl group,” or “heteroaromatic,” any of which terms include rings that are optionally substituted. The term “heteroaralkyl” refers to an alkyl group substituted by a heteroaryl, wherein the alkyl and heteroaryl portions independently are optionally substituted.

The term “heteroarylene” refers to a multivalent heteroaryl group having the appropriate number of open valences to account for groups attached to it. For example, “heteroarylene” is a bivalent heteroaryl group when it has two groups attached to it; “heteroarylene” is a trivalent heteroaryl group when it has three groups attached to it.

As used herein, the terms “heterocycle,” “heterocyclyl,” “heterocyclic radical,” and “heterocyclic ring” are used interchangeably and refer to a stable 5- to 7-membered monocyclic or 7-10-membered bicyclic heterocyclic moiety that is either saturated or partially unsaturated, and having, in addition to carbon atoms, one or more, preferably one to four, heteroatoms, as defined above. When used in reference to a ring atom of a heterocycle, the term “nitrogen” includes a substituted nitrogen. As an example, in a saturated or partially unsaturated ring having 0-3 heteroatoms selected from oxygen, sulfur or nitrogen, the nitrogen may be N (as in 3,4-dihydro-2H-pyrrolyl), NH (as in pyrrolidinyl), or +NR (as in N-substituted pyrrolidinyl).

A heterocyclic ring can be attached to its pendant group at any heteroatom or carbon atom that results in a stable structure and any of the ring atoms can be optionally substituted. Examples of such saturated or partially unsaturated heterocyclic radicals include, without limitation, tetrahydrofuranyl, tetrahydrothiophenyl pyrrolidinyl, piperidinyl, pyrrolinyl, tetrahydroquinolinyl, tetrahydroisoquinolinyl, decahydroquinolinyl, oxazolidinyl, piperazinyl, dioxanyl, dioxolanyl, diazepinyl, oxazepinyl, thiazepinyl, morpholinyl, 2-oxa-6-azaspiro[3.3]heptane, and quinuclidinyl. The terms “heterocycle,” “heterocyclyl,” “heterocyclyl ring,” “heterocyclic group,” “heterocyclic moiety,” and “heterocyclic radical,” are used interchangeably herein, and also include groups in which a heterocyclyl ring is fused to one or more aryl, heteroaryl, or cycloaliphatic rings, such as indolinyl, 3H-indolyl, chromanyl, phenanthridinyl, or tetrahydroquinolinyl. A heterocyclyl group may be mono- or bicyclic. The term “heterocyclylalkyl” refers to an alkyl group substituted by a heterocyclyl, wherein the alkyl and heterocyclyl portions independently are optionally substituted. The term “oxo-heterocyclyl” refers to a heterocyclyl substituted by one or more oxo group. The term “heterocyclylene” refers to a multivalent heterocyclyl group having the appropriate number of open valences to account for groups attached to it. For example, “heterocyclylene” is a bivalent heterocyclyl group when it has two groups attached to it; “heterocyclylene” is a trivalent heterocyclyl group when it has three groups attached to it. The term “oxo-heterocyclylene” refers to a multivalent oxo-heterocyclyl group having the appropriate number of open valences to account for groups attached to it.

As used herein, the term “partially unsaturated” refers to a ring moiety that includes at least one double or triple bond. The term “partially unsaturated” is intended to encompass rings having multiple sites of unsaturation, but is not intended to include aryl or heteroaryl moieties, as herein defined.

As described herein, compounds of the invention may contain “optionally substituted” moieties. In general, the term “substituted,” whether preceded by the term “optionally” or not, means that one or more hydrogens of the designated moiety are replaced with a suitable substituent. Unless otherwise indicated, an “optionally substituted” group may have a suitable substituent at each substitutable position of the group, and when more than one position in any given structure may be substituted with more than one substituent selected from a specified group, the substituent may be either the same or different at every position. Combinations of substituents envisioned by this invention are preferably those that result in the formation of stable or chemically feasible compounds. The term “stable,” as used herein, refers to compounds that are not substantially altered when subjected to conditions to allow for their production, detection, and, in certain embodiments, their recovery, purification, and use for one or more of the purposes disclosed herein.

Each optional substituent on a substitutable carbon is a monovalent substituent independently selected from halogen; —(CH₂)₀₋₄R°; —(CH₂)₀₋₄OR°; —O(CH₂)₀₋₄R°, —O—(CH₂)₀₋₄C(O)OR°; —(CH₂)₀₋₄CH(OR°)₂; —(CH₂)₀₋₄SR°; —(CH₂)₀₋₄Ph, which may be substituted with R°; —(CH₂)₀₋₄₀ (CH₂)₀₋₁Ph which may be substituted with R°: —CH—CHPh, which may be substituted with R°; —(CH₂)₀₋₄₀ (CH₂)₀₋₁-pyridyl which may be substituted with R°; —NO₂; —CN; —N₃; —(CH₂)₀₋₄N(R°)₂; —(CH₂)₀₋₄N(R°C(O)R°; —N(R°)C(S)R°; —(CH₂)₀₋₄N(R°)C(O)NR°₂;
—N(R°C(S)NR°₂; —(CH₂)₀₋₄N(R°)C(O)OR°; —N(R°N(R°C(O)R°; —N(R°N(R°)C(O)NR°₂; —N(R°N(R°C(O)OR°; —(CH₂)₀₋₄C(O)R°; —C(S)R°; —(CH₂)₀₋₄C(O)OR°; —(CH₂)₀₋₄C(O)SR°; —(CH₂)₀₋₄C(O)OSiR°₃; —(CH₂)₀₋₄OC(O)R°; —OC(O)(CH₂)₀₋₄SR—, SC(S)SR°; —(CH₂)₀₋₄SC(O)R°; —(CH₂)₀₋₄C(O)NR°₂; —C(S)NR°₂:—C(S)SR°; —SC(S)SR°, —(CH₂)₀₋₄OC(O)NR°₂; —C(O)N(OR°) R°; —C(O)C(O)R°; —C(O)CH₂C(O)R°; —C(NOR°) R°; —(CH₂)₀₋₄SSR°; —(CH₂)₀₋₄S(O)₂R°; —(CH₂)₀₋₄S(O)₂OR°; —(CH₂)₀₋₄OS(O)₂R°; —S(O)₂NR°₂; —S(O)(NR°) R°; —S(O)₂N═C(NR°₂)₂; —(CH₂)₀₋₄S(O)R°; —N(R°S(O)₂NR°₂; —N(R°S(O)₂R°; —N(OR°) R°; —C(NH)NR°₂; —P(O)₂R°; —P(O) R°)₂; —OP(O) R°)₂; —OP(O)(OR°)₂, SiR°₃; —C₁₋₄ straight or branched alkylene)O—N(R°)₂; or —(C₁₋₄ straight or branched alkylene) C(O)O—N(R°)₂.

Each R° is independently hydrogen, C₁₋₆ aliphatic, —CH₂Ph, —O(CH₂)₀₋₁Ph, —CH₂— (5-6 membered heteroaryl ring), or a 5-6-membered saturated, partially unsaturated, or aryl ring having 0-4 heteroatoms independently selected from nitrogen, oxygen, or sulfur, or, notwithstanding the definition above, two independent occurrences of R°, taken together with their intervening atom(s), form a 3-12-membered saturated, partially unsaturated, or aryl mono- or bicyclic ring having 0-4 heteroatoms independently selected from nitrogen, oxygen, or sulfur, which may be substituted by a divalent substituent on a saturated carbon atom of R° selected from ═O and ═S; or each R° is optionally substituted with a monovalent substituent independently selected from halogen, —(CH₂)_{0 2}R^{●}, -(haloR^{●}), —(CH₂)_{0 2}OH, —(CH₂)₀₋₂OR^{●}, —(CH₂)₀₋₂CH(OR^{●})₀₋₂; —O(haloR^{●}), —CN, —N₃, —(CH₂)₀₋₂C(O)R^{●}, —(CH₂)₀₋₂C(O)OH, —(CH₂)₀₋₂C(O)OR^{●}, —(CH₂)₀₋₂SR^{●}, —(CH₂)₀₋₂SH, —(CH₂)₀₋₂NH₂, —(CH₂)₀₋₂NHR^{●}, —(CH₂)₀₋₂NR^{●}₂, —NO₂, —SiR^{●}₃, —OSiR^{●}₃, —C(O) SR^{●}. —(C₁₋₄ straight or branched alkylene) C(O)OR^{●}, or —SSR^{●}.

Each R^{●} is independently selected from C₁₋₄ aliphatic, —CH₂Ph, —(CH₂)₀₋₁Ph, or a 5-6-membered saturated, partially unsaturated, or aryl ring having 0-4 heteroatoms independently selected from nitrogen, oxygen, or sulfur, and wherein each R^{●} is unsubstituted or where preceded by halo is substituted only with one or more halogens; or wherein an optional substituent on a saturated carbon is a divalent substituent independently selected from ═O, ═S, ═NNR*₂, ═NNHC(O)R*, ═NNHC(O)OR*, ═NNHS(O)₂R*, ═NR*, ═NOR*, —O(C(R*₂))₂₋₃O—, or —S(C(R*₂))₂₋₃S—, or a divalent substituent bound to vicinal substitutable carbons of an “optionally substituted” group is —O(CR*₂)₂₋₃O—, wherein each independent occurrence of R* is selected from hydrogen, Che aliphatic or an unsubstituted 5-6-membered saturated, partially unsaturated, or aryl ring having 0-4 heteroatoms independently selected from nitrogen, oxygen, or sulfur.

When R* is C₁₋₆ aliphatic, R* is optionally substituted with halogen, —R^{●}, -(haloR^{●}), —OH, —OR^{●}, —O(haloR^{●}), —CN, —C(O)OH, —C(O)OR^{●}, —NH₂, —NHR^{●}, —NR^{●}₂, or —NO₂, wherein each R^{●} is independently selected from C₁₋₄ aliphatic, —CH₂Ph, —O(CH₂)₀₋₁Ph, or a 5-6-membered saturated, partially unsaturated, or aryl ring having 0-4 heteroatoms independently selected from nitrogen, oxygen, or sulfur, and wherein each Re is unsubstituted or where preceded by halo is substituted only with one or more halogens.

An optional substituent on a substitutable nitrogen is independently —R^{†}, —NR^{†}₂, —C(O)R^{†}, —C(O)OR^{†}, —C(O)C(O)R^{†}, —C(O)CH₂C(O)R^{†}, —S(O)₂R^{†}, —S(O)₂NR^{†}₂, —C(S)NR^{†}₂, —C(NH)NR^{†}₂, or —N(R^{†})S(O)₂R^{†}; wherein each R^{†} is independently hydrogen, C₁₋₆ aliphatic, unsubstituted —OPh, or an unsubstituted 5-6-membered saturated, partially unsaturated, or aryl ring having 0-4 heteroatoms independently selected from nitrogen, oxygen, or sulfur, or, two independent occurrences of Rf, taken together with their intervening atom(s) form an unsubstituted 3-12-membered saturated, partially unsaturated, or aryl mono- or bicyclic ring having 0-4 heteroatoms independently selected from nitrogen, oxygen, or sulfur; wherein when R^{†} is C₁₋₆ aliphatic, R^{†} is optionally substituted with halogen, —R^{●}, -(haloR^{●}), —OH, —OR^{●}, —O(haloR^{●}), —CN, —C(O)OH, —C(O)OR^{●}, —NH₂, —NHR^{●}, —NR^{●}₂, or —NO₂, wherein each R^{●} is independently selected from C₁₋₄ aliphatic, —CH₂Ph, —O(CH₂)₀₋₁Ph, or a 5-6-membered saturated, partially unsaturated, or aryl ring having 0-4 heteroatoms independently selected from nitrogen, oxygen, or sulfur, and wherein each R^{●} is unsubstituted or where preceded by halo is substituted only with one or more halogens.

As used herein, the term “pharmaceutically acceptable salt” refers to those salts which are, within the scope of sound medical judgment, suitable for use in contact with the tissues of humans and lower animals without undue toxicity, irritation, allergic response and the like, and are commensurate with a reasonable benefit/risk ratio. Pharmaceutically acceptable salts are well known in the art. For example, S. M. Berge et al., describe pharmaceutically acceptable salts in detail in J. Pharmaceutical Sciences, 1977, 66, 1-19, incorporated herein by reference. Pharmaceutically acceptable salts of the compounds of this invention include those derived from suitable inorganic and organic acids and bases. Examples of pharmaceutically acceptable, nontoxic acid addition salts are salts of an amino group formed with inorganic acids such as hydrochloric acid, hydrobromic acid, phosphoric acid, sulfuric acid and perchloric acid or with organic acids such as acetic acid, oxalic acid, maleic acid, tartaric acid, citric acid, succinic acid or malonic acid or by using other methods used in the art such as ion exchange. Other pharmaceutically acceptable salts include adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, pivalate, propionate, stearate, succinate, sulfate, tartrate, thiocyanate, p-toluenesulfonate, undecanoate, valerate salts, and the like.

Further, acids which are generally considered suitable for the formation of pharmaceutically useful salts from basic pharmaceutical compounds are discussed, for example, by P. Stahl et al., Camille G. (eds.) Handbook of Pharmaceutical Salts. Properties, Selection and Use. (2002) Zurich: Wiley-VCH; S. Berge et al., Journal of Pharmaceutical Sciences (1977) 66 (1) 1-19; P. Gould, International J. of Pharmaceutics (1986) 33 201-217; Anderson et al., The Practice of Medicinal Chemistry (1996), Academic Press, New York; and in The Orange Book (Food & Drug Administration, Washington, D.C. on their website). These disclosures are incorporated herein by reference.

Salts derived from appropriate bases include alkali metal, alkaline earth metal. ammonium and N⁺ (C₁₋₄alkyl)₄ salts. Representative alkali or alkaline earth metal salts include sodium, lithium, potassium, calcium, magnesium, and the like. Further pharmaceutically acceptable salts include, when appropriate, nontoxic ammonium, quaternary ammonium, and amine cations formed using counterions such as halide, hydroxide, carboxylate, sulfate, phosphate, nitrate, loweralkyl sulfonate and aryl sulfonate.

Unless otherwise stated, structures depicted herein are also meant to include all isomeric (e.g., enantiomeric, diastereomeric, and geometric (or conformational) forms of the structure: for example, the R and S configurations for each asymmetric center, Z and E double bond isomers, and Z and E conformational isomers. Therefore, single stereochemical isomers as well as enantiomeric, diastereomeric, and geometric (or conformational) mixtures of the present compounds are within the scope of the invention. Unless otherwise stated, all tautomeric forms of the compounds of the invention are within the scope of the invention. The invention includes compounds that differ only in the presence of one or more isotopically enriched atoms. For example, compounds having the present structures including the replacement of hydrogen by deuterium or tritium, the replacement of a carbon by a ¹³C- or ¹⁴ C-enriched carbon, or the reaplacement of a fluorine by a ¹⁸F-enriched fluorine are within the scope of this invention. Such compounds are useful, for example, as analytical tools, as probes in biological assays, or as therapeutic agents in accordance with the present invention.

Diastereomeric mixtures can be separated into their individual diastereomers on the basis of their physical chemical differences by methods known to those skilled in the art, such as, for example, by chromatography and/or fractional crystallization. Enantiomers can be separated by converting the enantiomeric mixture into a diastereomeric mixture by reaction with an appropriate optically active compound (e.g., chiral auxiliary such as a chiral alcohol or Mosher's acid chloride), separating the diastereomers and converting (e.g., hydrolyzing) the individual diastereomers to the corresponding pure enantiomers. Alternatively, a particular enantiomer of a compound of the present invention may be prepared by asymmetric synthesis. Still further, where the molecule contains a basic functional group (such as amino) or an acidic functional group (such as carboxylic acid) diastereomeric salts are formed with an appropriate optically-active acid or base, followed by resolution of the diastereomers thus formed by fractional crystallization or chromatographic means known in the art, and subsequent recovery of the pure enantiomers.

Individual stereoisomers of the compounds of the invention may, for example, be substantially free of other isomers, or may be admixed, for example, as racemates or with all other, or other selected, stereoisomers. Chiral center(s) in a compound of the present invention can have the Sor R configuration as defined by the IUPAC 1974 Recommendations. Further, to the extent a compound described herein may exist as an atropisomer (e.g., substituted biaryls), all forms of such atropisomer are considered part of this invention.

Chemical names, common names, and chemical structures may be used interchangeably to describe the same structure. If a chemical compound is referred to using both a chemical structure and a chemical name, and an ambiguity exists between the structure and the name, the structure predominates. It should also be noted that any carbon as well as heteroatom with unsatisfied valences in the text, schemes, examples and tables herein is assumed to have the sufficient number of hydrogen atom(s) to satisfy the valences.

The terms “a” and “an” as used herein mean “one or more” and include the plural unless the context is inappropriate.

The term “alkyl” refers to a saturated straight or branched hydrocarbon, such as a straight or branched group of 1-12, 1-10, or 1-6 carbon atoms, referred to herein as C₁-C₁₂ alkyl, C₁-C₁₀ alkyl, and C₁-C₆ alkyl, respectively. Exemplary alkyl groups include, but are not limited to, methyl, ethyl, propyl, isopropyl, 2-methyl-1-propyl, 2-methyl-2-propyl, 2-methyl-1-butyl, 3-methyl-1-butyl, 2-methyl-3-butyl, 2,2-dimethyl-1-propyl, 2-methyl-1-pentyl, 3-methyl-1-pentyl, 4-methyl-1-pentyl, 2-methyl-2-pentyl, 3-methyl-2-pentyl, 4-methyl-2-pentyl, 2,2-dimethyl-1-butyl, 3,3-dimethyl-1-butyl, 2-ethyl-1-butyl, butyl, isobutyl, t-butyl, pentyl, isopentyl, neopentyl, hexyl, heptyl, octyl, etc.

The term “cycloalkyl” refers to a monovalent saturated cyclic, bicyclic, or bridged cyclic (e.g., adamantyl) hydrocarbon group of 3-12, 3-8, 4-8, or 4-6 carbons, referred to herein, e.g., as “C₃-C₆ cycloalkyl,” derived from a cycloalkane. Exemplary cycloalkyl groups include cyclohexyl, cyclopentyl, cyclobutyl, and cyclopropyl. The term “cycloalkylene” refers to a bivalent cycloalkyl group.

The term “haloalkyl” refers to an alkyl group that is substituted with at least one halogen. Exemplary haloalkyl groups include-CH₂F, —CHF₂, —CF₃, —CH₂CF₃, —CF₂CF₃, and the like. The term “haloalkylene” refers to a bivalent haloalkyl group.

The term “hydroxyalkyl” refers to an alkyl group that is substituted with at least one hydroxyl. Exemplary hydroxyalkyl groups include-CH₂CH₂OH, —C(H)(OH)CH₃, —CH₂C(H)(OH)CH₂CH₂OH, and the like.

The terms “alkenyl” and “alkynyl” are art-recognized and refer to unsaturated aliphatic groups analogous in length and possible substitution to the alkyls described above, but that contain at least one double or triple bond respectively.

The terms “alkoxyl” or “alkoxy” are art-recognized and refer to an alkyl group, as defined above, having an oxygen radical attached thereto. Representative alkoxyl groups include methoxy, ethoxy, propyloxy, tert-butoxy and the like. The term “haloalkoxyl” refers to an alkoxyl group that is substituted with at least one halogen. Exemplary haloalkoxyl groups include —OCH₂F, —OCHF₂, —OCF₃, —OCH₂CF₃, —OCF₂CF₃, and the like.

The term “oxo” is art-recognized and refers to a “═O” substituent. For example, a cyclopentane susbstituted with an oxo group is cyclopentanone.

The symbol “ [symbol] ” indicates a point of attachment.

When any substituent or variable occurs more than one time in any constituent or the compound of the invention, its definition on each occurrence is independent of its definition at every other occurrence, unless otherwise indicated.

One or more compounds of the invention may exist in unsolvated as well as solvated forms with pharmaceutically acceptable solvents such as water, ethanol, and the like, and it is intended that the invention embrace both solvated and unsolvated forms. “Solvate” means a physical association of a compound of this invention with one or more solvent molecules. This physical association involves varying degrees of ionic and covalent bonding, including hydrogen bonding. In certain instances the solvate will be capable of isolation, for example when one or more solvent molecules are incorporated in the crystal lattice of the crystalline solid. “Solvate” encompasses both solution-phase and isolatable solvates. Nonlimiting examples of suitable solvates include ethanolates, methanolates, and the like. “Hydrate” is a solvate wherein the solvent molecule is H₂O.

As used herein, the terms “subject” and “patient” are used interchangeable and refer to organisms to be treated by the methods of the present invention. Such organisms preferably include, but are not limited to, mammals (e.g., murines, simians, equines, bovines, porcines, canines, felines, and the like), and most preferably includes humans.

The term “IC₅₀” is art-recognized and refers to the concentration of a compound that is required to achieve 50% inhibition of the target.

As used herein, the term “effective amount” refers to the amount of a compound sufficient to effect beneficial or desired results (e.g., a therapeutic, ameliorative, inhibitory or preventative result). An effective amount can be administered in one or more administrations, applications or dosages and is not intended to be limited to a particular formulation or administration route. As used herein, the term “treating” includes any effect, e.g., lessening, reducing, modulating, ameliorating or eliminating, that results in the improvement of the condition, disease, disorder, and the like, or ameliorating a symptom thereof.

As used herein, the term “pharmaceutical composition” refers to the combination of an active agent with a carrier, inert or active, making the composition especially suitable for diagnostic or therapeutic use in vivo or ex vivo.

As used herein, the term “pharmaceutically acceptable carrier” refers to any of the standard pharmaceutical carriers, such as a phosphate buffered saline solution, water, emulsions (e.g., such as an oil/water or water/oil emulsions), and various types of wetting agents. The compositions also can include stabilizers and preservatives. For examples of carriers, stabilizers and adjuvants, see e.g., Martin, Remington's Pharmaceutical Sciences, 15th Ed., Mack Publ. Co., Easton, PA [1975].

For therapeutic use, salts of the compounds of the present invention are contemplated as being pharmaceutically acceptable. However, salts of acids and bases that are non-pharmaceutically acceptable may also find use, for example, in the preparation or purification of a pharmaceutically acceptable compound.

In addition, when a compound of the invention contains both a basic moiety (such as, but not limited to, a pyridine or imidazole) and an acidic moiety (such as, but not limited to, a carboxylic acid) zwitterions (“inner salts”) may be formed. Such acidic and basic salts used within the scope of the invention are pharmaceutically acceptable (i.e., non-toxic, physiologically acceptable) salts. Such salts of the compounds of the invention may be formed, for example, by reacting a compound of the invention with an amount of acid or base, such as an equivalent amount, in a medium such as one in which the salt precipitates or in an aqueous medium followed by lyophilization.

Throughout the description, where compositions are described as having, including, or comprising specific components, or where processes and methods are described as having, including, or comprising specific steps, it is contemplated that, additionally, there are compositions of the present invention that consist essentially of, or consist of, the recited components, and that there are processes and methods according to the present invention that consist essentially of, or consist of, the recited processing steps.

As a general matter, compositions specifying a percentage are by weight unless otherwise specified.

### I. Heterosubstituted Aminopyridinyl Imidazo[4,5-b]Pyridine & Related Compounds

One aspect of the invention provides aminopyridinyl imidazo[4,5-b]pyridine and related compounds. The compounds may be used in the pharmaceutical compositions and therapeutic methods described herein. Exemplary compounds are described in the following sections, along with exemplary procedures for making the compounds. One aspect of the invention provides a compound represented by Formula I:

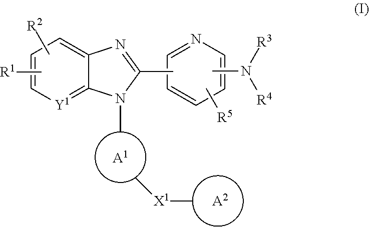

or a pharmaceutically acceptable salt thereof; wherein:
R¹ is phenyl, a 5-6 membered heteroaryl containing 1 or 2 heteroatoms selected from nitrogen, C₁₋₄ alkyl, C₁₋₄ haloalkyl, C₁₋₄ alkoxyl, C₁₋₄ haloalkoxy, cyano, C₃₋₄ cycloalkyl, —C(O)N(R¹⁰)(R¹¹), or hydrogen, wherein the phenyl and heteroaryl are each substituted with m occurrences of R¹³;
R², R³, R⁴, R⁵, and R⁹ are independently hydrogen or C₁₋₄ alkyl;
R⁶ is halo, C₁₋₄ haloalkyl, or C₁₋₄ alkyl;
R⁷ represents independently for each occurrence —(C₀₋₄ alkylene)-CO₂R⁹, hydroxyl, C₁₋₄ alkoxyl, C₁₋₄ alkyl, C₁₋₄ haloalkyl, cyano, —(C₀₋₄ alkylene)-C(O)N(R¹⁰)(R¹¹), —(C₀₋₄ alkylene)-N(R¹⁰)C(O)R¹², —SO₃R¹¹, —S(O)₂N(R¹⁰)(R¹¹), —S(O)₂R¹², or —N(R¹⁰)(R¹¹);
R⁸ represents independently for each occurrence hydroxyl, C₁₋₄ alkoxyl, C₁₋₄ alkyl, C₁₋₄ haloalkyl, halo, cyano, or —N(R¹⁰)(R¹¹);
R¹⁰ and R¹¹ each represent independently for each occurrence hydrogen or C₁₋₄ alkyl, or R¹⁰ and R¹¹ are taken together with the nitrogen atom to which they are attached to form a 3-7 membered ring;
R¹² represents independently for each occurrence C₁₋₆ alkyl or C₃₋₆ cycloalkyl;
R¹³ represents independently for each occurrence halo, C₁₋₄ alkyl, —(C₀₋₄ alkylene)-C(O)N(R¹⁰)(R¹¹), —(C₀₋₄ alkylene)-N(R¹⁰) C(O)R¹², —C(O)N(R¹⁰)-phenyl, or —N(R¹⁰)C(O)— phenyl;
A¹ is phenylene or pyridinylene, each of which is substituted with 0 or 1 occurrence of R⁶;
A² is phenyl or pyridinyl, each of which is substituted with (i) 1 occurrence of R⁷ and (ii) n occurrences of R⁸;
X¹ is —N(R⁹)C(O)-Ψ, —C(O)N(R⁹)-Ψ, —(C₁₋₃ alkylene)-N(R⁹)C(O)-Ψ, -(3-6 membered saturated heterocyclylene containing 1 or 2 heteroatoms selected from nitrogen)-C(O)-Ψ, —N(R⁹)C(R¹⁰)(R¹¹)-Ψ, —C(R⁹)(R¹⁰)N(R⁹)C(R⁹)(R¹⁰)—, —C(O)N(R⁹)—(C₁₋₃ alkylene)-Ψ, or -(3-6 membered saturated heterocyclylene containing 1 or 2 heteroatoms selected from nitrogen)-C(R¹⁰)(R¹¹)-Ψ; wherein Ψ is a bond to A²;
Y¹ is N or —C(H)—; and
m and n are independently 0, 1, or 2

In some embodiments, the compound is represented by Formula I:

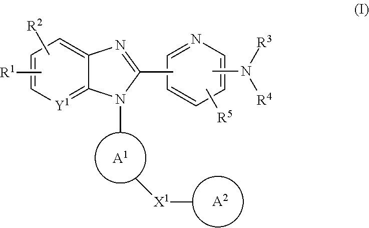

or a pharmaceutically acceptable salt thereof; wherein:
R¹ is phenyl, a 5-6-membered heteroaryl containing 1 or 2 heteroatoms selected from nitrogen, C₁₋₄ haloalkyl, C₁₋₄ alkoxyl, cyano, C₃₋₄ cycloalkyl, —C(O)N(R¹⁰)(R¹³), or hydrogen, wherein the phenyl and heteroaryl are each substituted with m occurrences of R¹³;
R², R³, R⁴, R⁵, and R⁹ are independently hydrogen or C₁₋₄ alkyl;
R⁶ is halo, C₁₋₄ haloalkyl, or C₁₋₄ alkyl;
R⁷ represents independently for each occurrence-(C₀₋₄ alkylene)-CO₂R⁹, hydroxyl, C₁₋₄ alkoxyl, C₁₋₄ alkyl, C₁₋₄ haloalkyl, cyano, —(C₀₋₄ alkylene)-C(O)N(R¹⁰)(R¹¹), —(C₀₋₄ alkylene)-N(R¹⁰)C(O)R¹², —SO₃R¹¹, —S(O)₂N(R¹⁰)(R¹¹), —S(O)₂R¹², or —N(R¹⁰)(R¹¹);
R⁸ represents independently for each occurrence hydroxyl, C₁₋₄ alkoxyl, C₁₋₄ alkyl, C₁₋₄ haloalkyl, halo, cyano, or —N(R¹⁰)(R¹¹);
R¹⁰ and R¹¹ each represent independently for each occurrence hydrogen or C₁₋₄ alkyl, or R¹⁰ and R¹¹ are taken together with the nitrogen atom to which they are attached to form a 3-7 membered ring;
R¹² represents independently for each occurrence C₁₋₆ alkyl or C₃₋₆ cycloalkyl;
R¹³ represents independently for each occurrence halo, C₁₋₄ alkyl, —(C₀₋₄ alkylene)-C(O)N(R¹⁰)(R¹¹), —(C₀₋₄ alkylene)-N(R¹⁰)C(O)R¹², —C(O)N(R¹⁰)-phenyl, or —N(R¹⁰)C(O)-phenyl;
A¹ is phenylene or pyridinylene, each of which is substituted with 0 or 1 occurrence of R⁶;
A² is phenyl or pyridinyl, each of which is substituted with (i) 1 occurrence of R⁷ and (ii) n occurrences of R⁸;
X¹ is —N(R⁹)C(O)-Ψ, —(C₁₋₃ alkylene)-N(R⁹)C(O)-Ψ, -(3-6 membered saturated heterocyclylene containing 1 or 2 heteroatoms selected from nitrogen)-C(O)-Ψ, —N(R⁹)C(R¹⁰)(R¹¹)-4, —C(R⁹)(R¹⁰)N(R⁹)C(R⁹)(R¹⁰)—, —C(O)N(R⁹)—(C₁₋₃ alkylene)-Ψ, or -(3-6 membered saturated heterocyclylene containing 1 or 2 heteroatoms selected from nitrogen)-C(R¹⁰)(R¹¹)-Ψ; wherein Ψ is a bond to A²;
Y¹ is N or —C(H)—; and
m and n are independently 0, 1, or 2.

The definitions of variables in Formula I above encompass multiple chemical groups. The application contemplates embodiments where, for example, i) the definition of a variable is a single chemical group selected from those chemical groups set forth above, ii) the definition of a variable is a collection of two or more of the chemical groups selected from those set forth above, and iii) the compound is defined by a combination of variables in which the variables are defined by (i) or (ii).

In certain embodiments, the compound is a compound of Formula I.

As generally defined above, R¹ is phenyl, a 5-6 membered heteroaryl containing 1 or 2 heteroatoms selected from nitrogen, C₁₋₄ alkyl, C₁₋₄ haloalkyl, C₁₋₄ alkoxyl, C₁₋₄ haloalkoxy, cyano, C₃₋₄ cycloalkyl, —C(O)N(R¹⁰)(R¹¹), or hydrogen, wherein the phenyl and heteroaryl are each substituted with m occurrences of R¹³. In certain embodiments, R′ is phenyl, a 5-6-membered heteroaryl containing 1 or 2 heteroatoms selected from nitrogen, C₁₋₄ haloalkyl, C₁₋₄ alkoxyl, cyano, C₃₋₄ cycloalkyl, —C(O)N(R¹⁰)(R¹¹), or hydrogen, wherein the phenyl and heteroaryl are each substituted with m occurrences of R¹³. In certain embodiments, R¹ is phenyl substituted with m occurrences of R¹³. In certain embodiments, R¹ is a 5-6-membered heteroaryl containing 1 or 2 heteroatomns selected from nitrogen substituted with m occurrences of R¹³. In certain embodiments, R¹ is C₁₋₄ haloalkyl. In certain embodiments, R¹ is C₁₋₄ alkoxyl. In certain embodiments. R¹ is cyano. In certain embodiments, R¹ is C₃₋₄ cycloalkyl. In certain embodiments, R¹ is —C(O)N(R¹⁰)(R¹¹). In certain embodiments. R¹ is hydrogen. In certain embodiments, R¹ is CF₃. In certain embodiments, R¹ is —C(O)NH₂. In certain embodiments. R¹ is C₁₋₄ alkyl. In certain embodiments, R¹ is haloalkoxy.

In certain embodiments, R¹ is phenyl. In certain embodiments, R¹ is C₁₋₄ haloalkyl, C₁₋₄ alkoxyl, or hydrogen. In certain embodiments, R¹ is selected from the groups depicted in the compounds in Table 1, below.

As generally defined above, R², R³, R⁴, R⁵, and R⁹ are independently hydrogen or C₁₋₄ alkyl. In certain embodiments, R² is hydrogen or C₁₋₄ alkyl. In certain embodiments, R³ is hydrogen or C₁₋₄ alkyl. In certain embodiments, R⁴ is hydrogen or C₁₋₄ alkyl. In certain embodiments, R⁵ is hydrogen or C₁₋₄ alkyl. In certain embodiments, R⁹ is hydrogen or C₁₋₄ alkyl. In certain embodiments, R² is C₁₋₄ alkyl. In certain embodiments, R³ is C₁₋₄ alkyl. In certain embodiments, R⁴ is C₁₋₄ alkyl. In certain embodiments, R⁵ is C₁₋₄ alkyl. In certain embodiments, R⁹ is C₁₋₄ alkyl. In certain embodiments, R², R³, R⁴, R⁵, and R⁹ are hydrogen. In certain embodiments, R², R³, R⁴, R⁵, and R⁹ are C₁₋₄ alkyl.

In certain embodiments, R² is hydrogen. In certain embodiments, R³ is hydrogen. In certain embodiments, R⁴ is hydrogen. In certain embodiments. R⁵ is hydrogen. In certain embodiments, R⁹ is hydrogen.

In certain embodiments. R² is selected from the groups depicted in the compounds in Table 1, below. In certain embodiments, R³ is selected from the groups depicted in the compounds in Table 1, below. In certain embodiments. R⁴ is selected from the groups depicted in the compounds in Table 1, below. In certain embodiments, R⁵ is selected from the groups depicted in the compounds in Table 1, below. In certain embodiments, R⁹ is selected from the groups depicted in the compounds in Table 1, below.

As generally defined above, Re is halo, C₁₋₄ haloalkyl, or C₁₋₄ alkyl. In certain embodiments, R⁶ is halo or C₁₋₄ alkyl. In certain embodiments, R⁶ is halo. In certain embodiments, R⁶ is C₁₋₄ alkyl. In certain embodiments, R⁶ is C₁₋₄ haloalkyl. In certain embodiments. R⁶ is halo or C₁₋₄ alkyl. In certain embodiments, R⁶ is selected from the groups depicted in the compounds in Table 1, below.

As generally defined above, R⁷ represents independently for each occurrence-(C₀₋₄ alkylene)-CO₂R⁹, hydroxyl, C₁₋₄ alkoxyl, C₁₋₄ alkyl, C₁₋₄ haloalkyl, cyano, —(C₀₋₄ alkylene)-C(O)N(R¹⁰)(R¹¹), —(C₀₋₄ alkylene)-N(R¹⁰)C(O)R¹², —SO₂R¹¹, —S(O)₂N(R¹⁰)(R¹¹), —S(O)₂R¹², or —N(R¹⁰)(R¹¹). In certain embodiments, R⁷ represents independently for each occurrence-(C₀₋₄ alkylene)-CO₂R⁹. In certain embodiments, R⁷ represents independently for each occurrence hydroxyl. In certain embodiments, R⁷ represents independently for each occurrence C₁₋₄ alkoxyl. In certain embodiments, R⁷ represents independently for each occurrence C₁₋₄ alkyl. In certain embodiments, R⁷ represents independently for each occurrence C₁₋₄ haloalkyl. In certain embodiments, R⁷ represents independently for each occurrence cyano. In certain embodiments, R⁷ represents independently for each occurrence-(C₀₋₄ alkylene)-C(O)N(R¹⁰)(R¹¹). In certain embodiments, R⁷ represents independently for each occurrence-(C₀₋₄ alkylene)-N(R¹⁰) C(O)R¹². In certain embodiments, R⁷ represents independently for each occurrence —SO₃R¹¹. In certain embodiments. R⁷ represents independently for each occurrence-S(O)₂N(R¹⁰)(R¹¹). In certain embodiments, R⁷ represents independently for each occurrence-S(O)₂R¹². In certain embodiments, R⁷ represents independently for each occurrence-N(R¹⁰)(R¹¹).

In certain embodiments, R⁷ is —(C₀₋₄ alkylene)-CO₂R⁹. In certain embodiments, R⁷ is hydroxyl. In certain embodiments, R⁷ is C₁₋₄ alkoxyl. In certain embodiments, R⁷ is C₁₋₄ alkyl. In certain embodiments, R⁷ is C₁₋₄ haloalkyl. In certain embodiments, R⁷ is cyano. In certain embodiments, R⁷ is —(C₀₋₄ alkylene)-C(O)N(R¹⁰)(R¹¹). In certain embodiments, R⁷ is —(C₀₋₄ alkylene)-N(R¹⁰) C(O)R¹². In certain embodiments, R⁷ is —SO₃R¹¹. In certain embodiments, R⁷ is-S(O)₂N(R¹⁰)(R¹¹). In certain embodiments, R⁷ is-S(O)₂R¹². In certain embodiments, R⁷ is or —N(R¹⁰)(R¹¹).

In certain embodiments, R⁷ is —CO₂H. In certain embodiments, R⁷ is —CH₂CO₂H. In certain embodiments, R⁷ is —CH₂CH₂CO₂H. In certain embodiments, R⁷ is-C(H)(CH₃)CO₂H. In certain embodiments, R⁷ is —(C₀₋₄ alkylene)-C(O)N(R¹⁰)(R¹¹), —(C₀₋₄ alkylene)-N(R¹⁰)C(O)R¹², or —N(R¹⁰)(R¹¹). In certain embodiments, R⁷ is-SO₃R¹¹, —S(O)₂N(R¹⁰)(R¹¹), or —S(O)₂R¹².

In certain embodiments. R⁷ is selected from the groups depicted in the compounds in Table 1, below.

As generally defined above, R⁸ represents independently for each occurrence hydroxyl, C₁₋₄ alkoxyl, C₁₋₄ alkyl, C₁₋₄ haloalkyl, halo, cyano, or —N(R¹⁰)(R¹¹). In certain embodiments, R⁸ represents for each occurrence hydroxyl. In certain embodiments. R⁸ represents for each occurrence C₁₋₄ alkoxyl. In certain embodiments, R⁸ represents for each occurrence C₁₋₄ alkyl. In certain embodiments, R⁸ represents for each occurrence C₁₋₄ haloalkyl. In certain embodiments. R⁸ represents for each occurrence halo. In certain embodiments, R⁸ represents for each occurrence cyano. In certain embodiments, R⁸ represents for each occurrence-N(R¹⁰)(R¹¹). In certain embodiments, R⁸ is hydroxyl. In certain embodiments, R⁸ is C₁₋₄ alkoxyl. In certain embodiments, R⁸ is C₁₋₄ alkyl. In certain embodiments, R⁸ is C₁₋₄ haloalkyl. In certain embodiments, R³ is halo. In certain embodiments, R³ is cyano. In certain embodiments, R⁸ is —N(R¹⁰)(R¹¹).

In certain embodiments, R⁸ represents independently for each occurrence hydroxyl, C₁₋₄ alkoxyl, and C₁₋₄ alkyl. In certain embodiments, R⁸ is hydroxyl. In certain embodiments, R⁸ represents independently for each occurrence C₁₋₄ alkyl. In certain embodiments, R⁸ is methyl. In certain embodiments, R⁸ represents independently for each occurrence C₁₋₄ alkoxyl. In certain embodiments, R⁸ is methoxy.

In certain embodiments, R⁸ is selected from the groups depicted in the compounds in Table 1, below.

As generally defined above, R¹⁰ and R¹¹ each represent independently for each occurrence hydrogen or C₁₋₄ alkyl, or R¹⁰ and R¹¹ are taken together with the nitrogen atom to which they are attached to form a 3-7 membered ring. In certain embodiments, R¹⁰ and R¹¹ are hydrogen. In certain embodiments. R¹⁰ and R¹¹ are hydrogen or C₁₋₄ alkyl. In certain embodiments, R¹⁰ is hydrogen. In certain embodiments, R¹⁰ is C₁₋₄ alkyl. In certain embodiments, R¹¹ is hydrogen. In certain embodiments, R¹¹ is C₁₋₄ alkyl. In certain embodiments, R¹⁰ and R¹¹ are taken together with the nitrogen atom to which they are attached to form a 3-7 membered ring.

In certain embodiments, R¹⁰ is selected from the groups depicted in the compounds in Table 1, below. In certain embodiments, R¹¹ is selected from the groups depicted in the compounds in Table 1, below.

As generally defined above, R¹² represents independently for each occurrence C₁₋₆ alkyl or C₃₋₆ cycloalkyl. In certain embodiments, R¹² represents independently for each occurrence C₁₋₆ alkyl. In certain embodiments, R¹² represents independently for each occurrence C₃₋₆ cycloalkyl. In certain embodiments. R¹² is C₁₋₆ alkyl. In certain embodiments, R¹² is C₃₋₆ cycloalkyl. In certain embodiments. R¹² is selected from the groups depicted in the compounds in Table 1, below.

As generally defined above, R¹³ represents independently for each occurrence halo, C₁₋₄ alkyl, —(C₀₋₄ alkylene)-C(O)N(R¹⁰)(R¹¹), —(C₀₋₄)alkylone)-N(R¹⁰)C(O)R¹², —C(O)N(R¹⁰)-phenyl, or —N(R¹⁰)C(O)-phenyl. In certain embodiments, R¹³ represents independently for each occurrence halo. In certain embodiments, R¹³ represents independently for each occurrence C₁₋₄ alkyl. In certain embodiments. R¹³ represents independently for each occurrence-(C₀₋₄ alkylene)-C(O)N(R¹⁰)(R¹¹). In certain embodiments, R¹³ represents independently for each occurrence-(C₀₋₄ alkylene)-N(R¹⁰) C(O)R¹². In certain embodiments, R¹³ represents independently for each occurrence-C(O)N(R¹⁰)-phenyl. In certain embodiments, R¹³ represents independently for each occurrence-N(R¹⁰)C(O)-phenyl. In certain embodiments, R¹³ is halo. In certain embodiments, R¹³ is C₁₋₄ alkyl. In certain embodiments, R¹³ is —(C₀₋₄ alkylene)-C(O)N(R¹⁰)(R¹¹). In certain embodiments, R¹³ is ~ (C₀₋₄ alkylene)-N(R¹⁰) C(O)R¹². In certain embodiments, R¹³ is —C(O)N(R¹⁰)-phenyl. In certain embodiments, R¹³ is —N(R¹)C(O)-phenyl. In certain embodiments, R¹³ is selected from the groups depicted in the compounds in Table 1, below.

As generally defined above, A¹ is phenylene or pyridinylene, each of which is substituted with 0 or 1 occurrence of R⁶. In certain embodiments, A¹ is phenylene substituted with 0 or 1 occurrence of R⁶. In certain embodiments, A¹ is phenylene substituted with 1 occurrence of R⁶. In certain embodiments, A¹ is phenylene. In certain embodiments, A¹ is pyridinylene. In certain embodiments, A¹ is selected from the groups depicted in the compounds in Table 1. below.

As generally defined above, A² is phenyl or pyridinyl, each of which is substituted with (i) 1 occurrence of R⁷ and (ii) n occurrences of R⁸. In certain embodiments, A² is phenyl substituted with (i) 1 occurrence of R⁷ and (ii) n occurrences of R⁸. In certain embodiments, A² is pyridinyl substituted with (i) 1 occurrence of R⁷ and (ii) n occurrences of R⁸. In certain embodiments, A² is phenyl substituted with (i) 1 occurrence of R⁷. In certain embodiments, A² is phenyl substituted with n occurrences of R⁸. In certain embodiments, A² is one of the following:

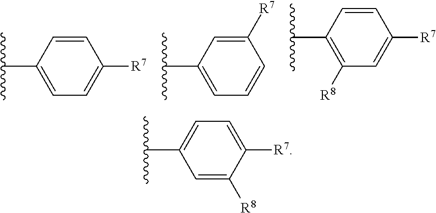

In certain embodiments, A² is selected from the groups depicted in the compounds in Table 1, below.

As generally defined above, X¹ is —N(R⁹)C(O)-Ψ, —C(O)N(R⁹)-Ψ, —(C₁₋₃ alkylene)-N(R⁹)C(O)-Ψ, -(3-6 membered saturated heterocyclylene containing 1 or 2 heteroatoms selected from nitrogen)-C(O)—, —N(R⁹)C(R¹⁰)(R¹³)-Ψ, —C(R⁹)(R¹⁰) N(R⁹)C(R⁹)(R¹⁰)—, —C(O)N(R⁹)—(C₁₋₃ alkylene)-Ψ, or -(3-6 membered saturated heterocyclylene containing 1 or 2 heteroatoms selected from nitrogen)-C(R¹⁰)(R¹¹)-4′; wherein Y is a bond to A². In certain embodiments, X¹ is —N(R⁹)C(O)-Ψ, —(C₁₋₃ alkylene)-N(R⁹)C(O)-Ψ, -(3-6 membered saturated heterocyclylene containing 1 or 2 heteroatoms selected from nitrogen)-C(O)-Ψ, —N(R⁹)C(R¹⁰)(R¹¹)-Ψ, —C(R⁹)(R¹⁰)N(R⁹)C(R⁹)(R¹⁰)—, —C(O)N(R⁹)—(C₁₋₃ alkylene)-Ψ, or -(3-6 membered saturated heterocyclylene containing 1 or 2 heteroatoms selected from nitrogen)-C(R¹⁰)(R¹¹)-Ψ; wherein Ψ is a bond to A². In certain embodiments, X¹ is —N(R⁹)C(O)-Ψ, wherein Ψ is a bond to A². In certain embodiments, X¹ is —C(O)N(R⁹)-Ψ, wherein Y is a bond to A². In certain embodiments, X¹ is —(C₁₋₃ alkylene)-N(R⁹)C(O)—, wherein Ψ is a bond to A². In certain embodiments, X¹ is -(3-6 membered saturated heterocyclylene containing 1 or 2 heteroatoms selected from nitrogen)-C(O)-Ψ; wherein Ψ is a bond to A². In certain embodiments, X¹ is —N(R⁹)C(O)-Ψ. In certain embodiments, X¹ is —C(O)N(R⁹)-Ψ. In certain embodiments, X¹ is —(C₁₋₃ alkylene)-N(R⁹)C(O)-Ψ. In certain embodiments, X¹ is —(CH₂)—N(R⁹)C(O)-Ψ. In certain embodiments, X¹ is)-C(R⁹)(R¹⁰)N(R⁹)C(R⁹)(R¹⁰)—. In certain embodiments, X¹ is —C(O)N(R⁹)—(C₁₋₃ alkylene)-Ψ. In certain embodiments, X¹ is a -(3-6 membered saturated heterocyclylene containing 1 or 2 heteroatoms selected from nitrogen)-C(O)-Ψ. In certain embodiments, X¹ is a -(4-membered saturated heterocyclylene containing 1 heteroatom selected from nitrogen)-C(O)-Ψ. In certain embodiments, X¹ is

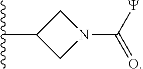

In certain embodiments, X¹ is selected from the groups depicted in the compounds in Table 1, below.

As generally defined above, Y¹ is N or —C(H)—. In certain embodiments, Y′ is N. In certain embodiments, Y′ is —C(H)—. In certain embodiments, Y¹ is selected from the groups depicted in the compounds in Table 1, below.

As generally defined above, m and n are independently 0, 1, or 2. In certain embodiments, n is 1. In certain embodiments, n is 0. In certain embodiments, n is 2.

In certain embodiments, m is 1. In certain embodiments, m is 2. In certain embodiments, m is 0.

In certain embodiments, m is a value according to that depicted in the compounds in Table 1, below. In certain embodiments, n is a value according to that depicted in the compounds in Table 1, below.

In certain embodiments, the compound is represented by Formula I-aa, I-ab, I-ac, I-ad, I-ae, I-af, I-ag, I-ah, or I-ai, or a pharmaceutically acceptable salt thereof:

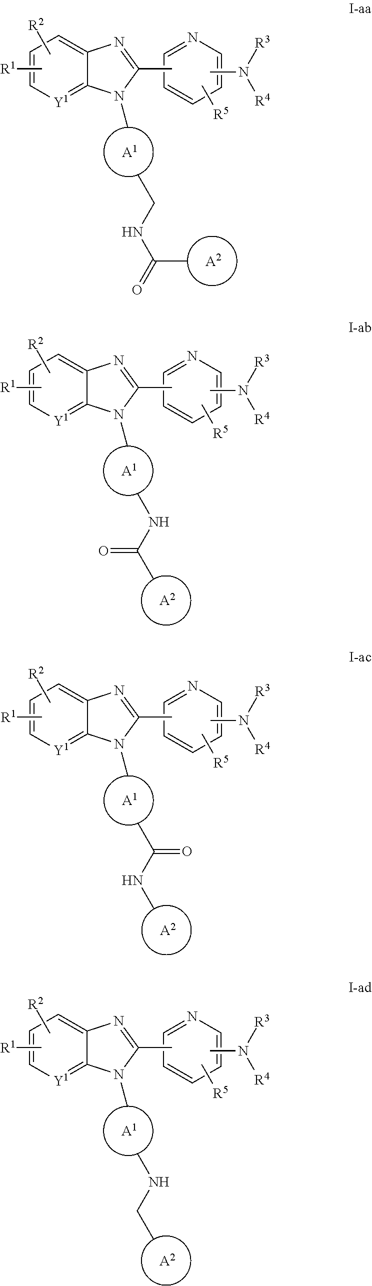

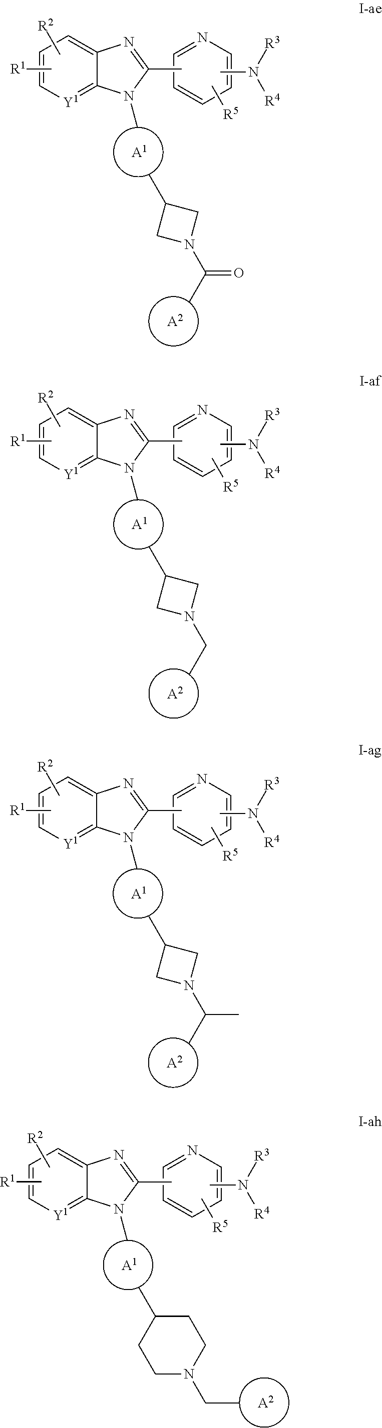

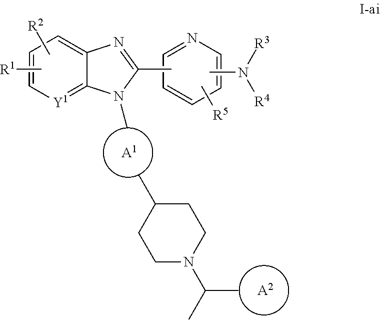

In certain embodiments, the compound is represented by Formula I-aa, I-ab, I-af, or I-ag, or a pharmaceutically acceptable salt thereof.

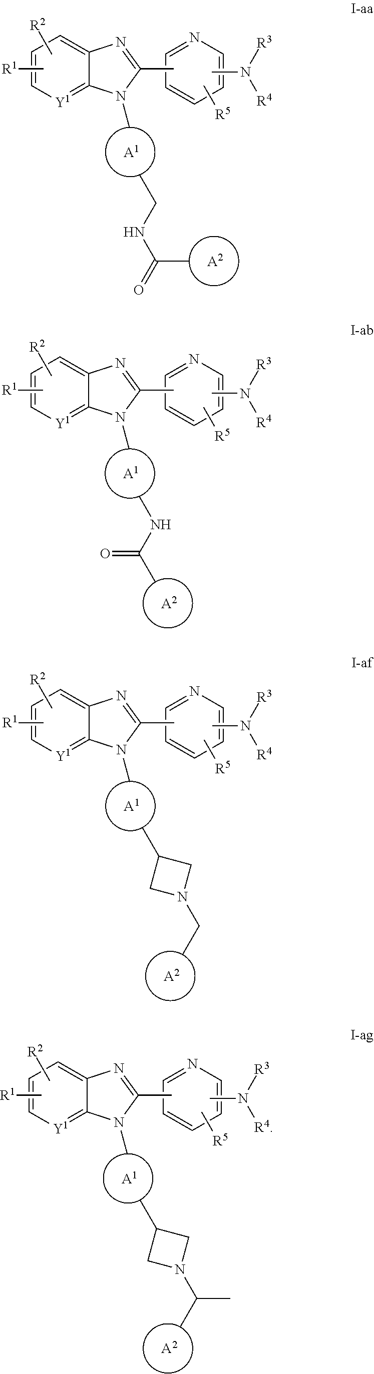

In certain embodiments, the compound is represented by Formula Ta or a pharmaceutically acceptable salt thereof:

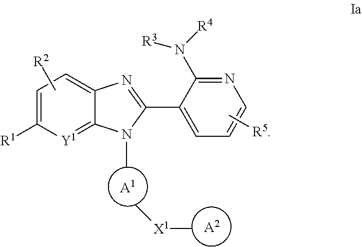

In certain embodiments, the compound is a compound of Formula Ib, Ic, Id, Ie, If, or Ig, or a pharmaceutically acceptable salt thereof:

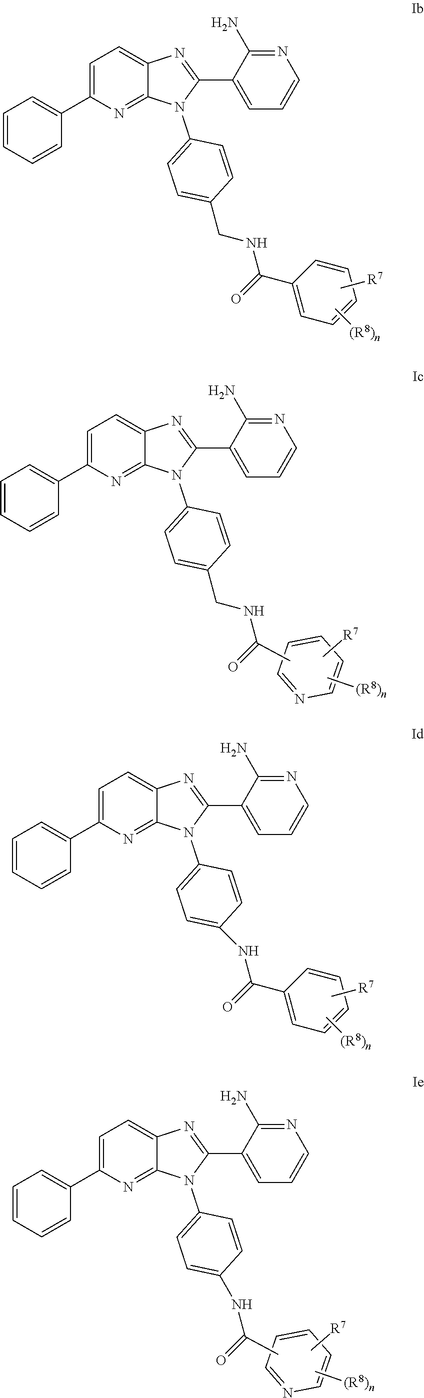

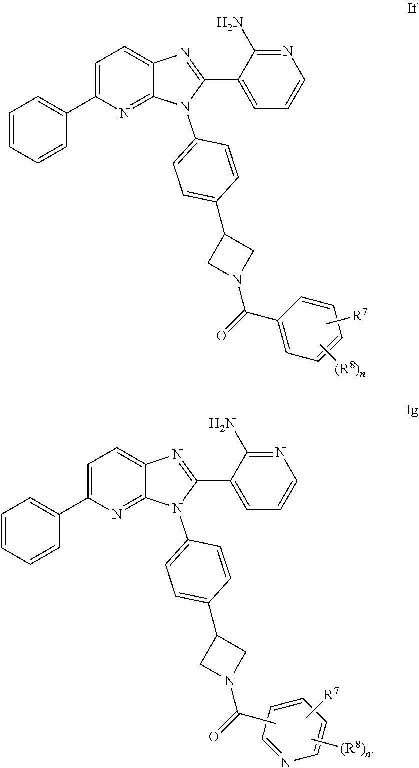

In certain embodiments, the compound is a compound of Formula Ih, Ii, Ij, Ik, Il, or Im, or a pharmaceutically acceptable salt thereof.

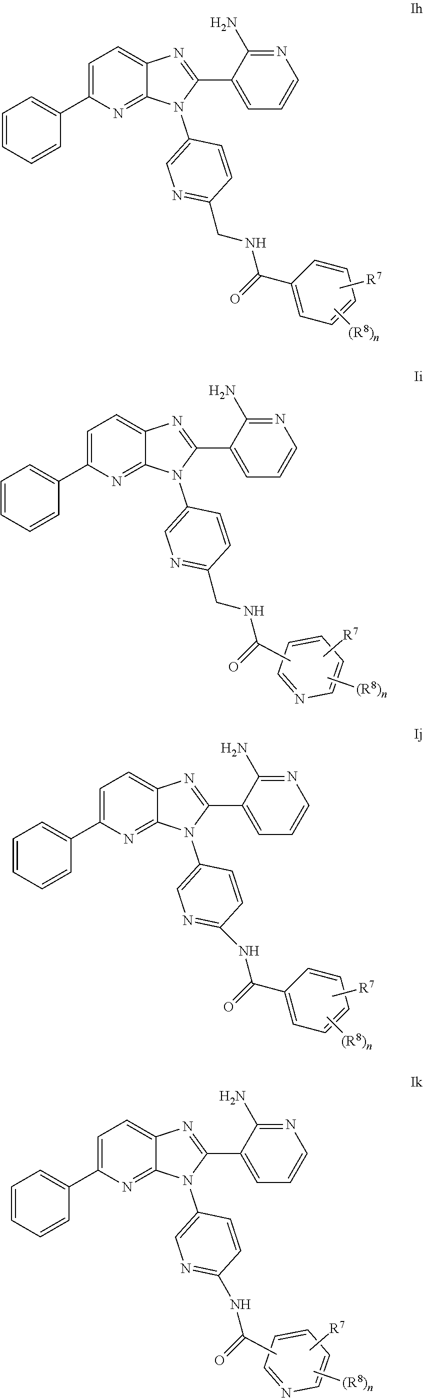

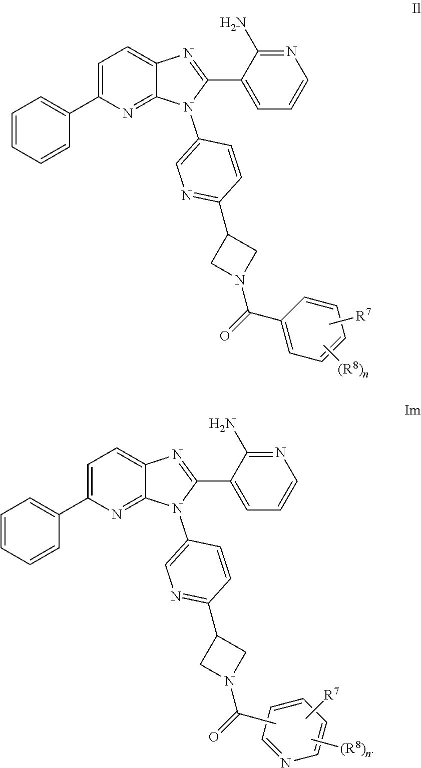

### Exemplary Specific Compounds

In certain embodiments, the compound is a compound in Table 1 or a stereoisomer or pharmaceutically acceptable salt thereof. In certain embodiments, the compound is a compound in Table 1. It is contemplated herein, in certain embodiments, that any free carboxylic acid present in a compound in Table 1 could be readily replaced with a corresponding ester group (e.g. a methyl ester). It is also contemplated herein, in certain embodiments, that any ester group (e.g. a methyl ester) present in a compound in Table 1 could be readily replaced with a free carboxylic acid.

TABLE 1

| Compound No. | Chemical Structure |
|---|---|
| 1 | 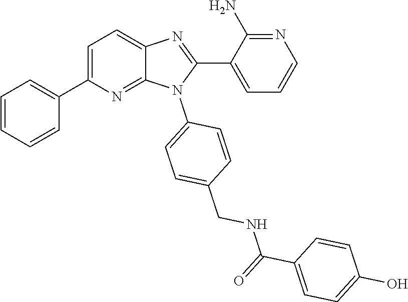 [SMILES: Nc1ncccc1-c1nc2ccc(-c3ccccc3)nc2n1-c1ccc(CNC(=O)c2ccc(O)cc2)cc1] |
| 2 | 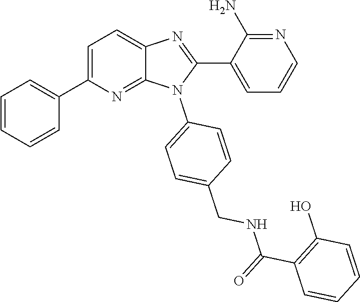 [SMILES: Nc1ncccc1-c1nc2ccc(-c3ccccc3)nc2n1-c1ccc(CNC(=O)c2ccccc2O)cc1] |
| 3 | 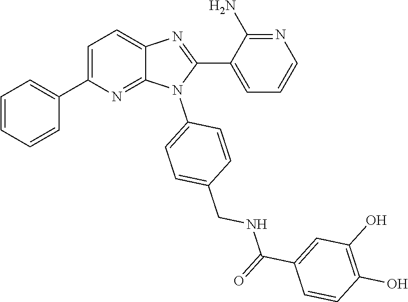 [SMILES: Nc1ncccc1-c1nc2ccc(-c3ccccc3)nc2n1-c1ccc(CNC(=O)c2ccc(O)c(O)c2)cc1] |
| 4 | 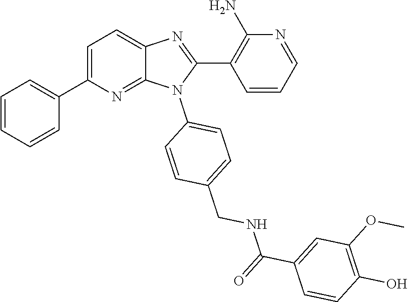 [SMILES: COc1cc(C(=O)NCc2ccc(-n3c(-c4cccnc4N)nc4ccc(-c5ccccc5)nc43)cc2)ccc1O] |
| 5 | 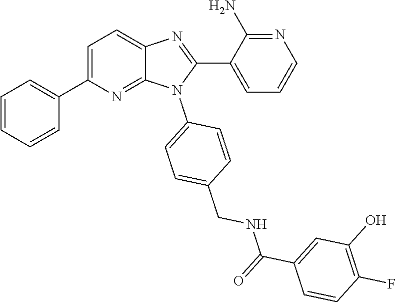 [SMILES: Nc1ncccc1-c1nc2ccc(-c3ccccc3)nc2n1-c1ccc(CNC(=O)c2ccc(F)c(O)c2)cc1] |
| 6 | 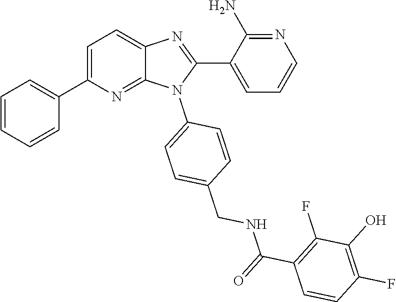 [SMILES: Nc1ncccc1-c1nc2ccc(-c3ccccc3)nc2n1-c1ccc(CNC(=O)c2ccc(F)c(O)c2F)cc1] |
| 7 | 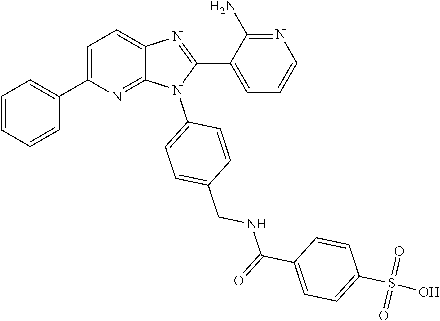 [SMILES: Nc1ncccc1-c1nc2ccc(-c3ccccc3)nc2n1-c1ccc(CNC(=O)c2ccc(S(=O)(=O)O)cc2)cc1] |
| 8 | 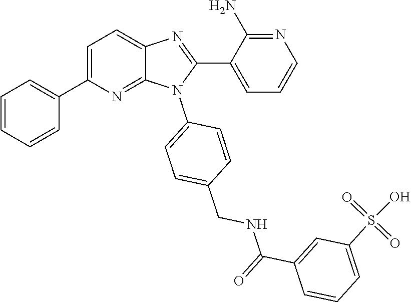 [SMILES: Nc1ncccc1-c1nc2ccc(-c3ccccc3)nc2n1-c1ccc(CNC(=O)c2cccc(S(=O)(=O)O)c2)cc1] |
| 9 | 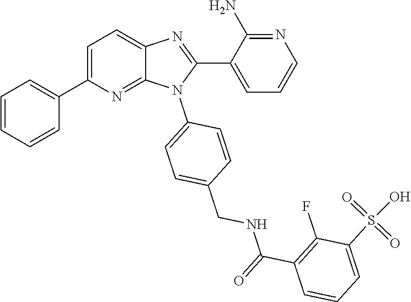 [SMILES: Nc1ncccc1-c1nc2ccc(-c3ccccc3)nc2n1-c1ccc(CNC(=O)c2cccc(S(=O)(=O)O)c2F)cc1] |
| 10 | 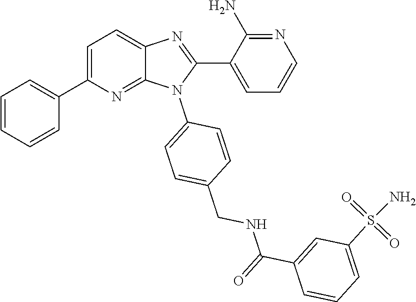 [SMILES: Nc1ncccc1-c1nc2ccc(-c3ccccc3)nc2n1-c1ccc(CNC(=O)c2cccc(S(N)(=O)=O)c2)cc1] |
| 11 | 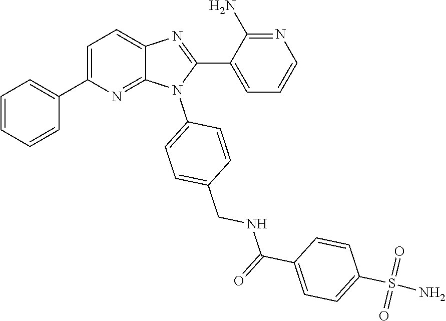 [SMILES: Nc1ncccc1-c1nc2ccc(-c3ccccc3)nc2n1-c1ccc(CNC(=O)c2ccc(S(N)(=O)=O)cc2)cc1] |
| 12 | 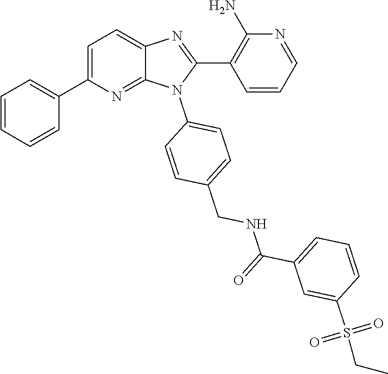 [SMILES: CCS(=O)(=O)c1cccc(C(=O)NCc2ccc(-n3c(-c4cccnc4N)nc4ccc(-c5ccccc5)nc43)cc2)c1] |
| 13 | 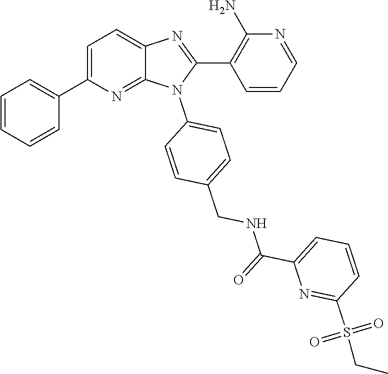 [SMILES: CCS(=O)(=O)c1cccc(C(=O)NCc2ccc(-n3c(-c4cccnc4N)nc4ccc(-c5ccccc5)nc43)cc2)n1] |
| 14 | 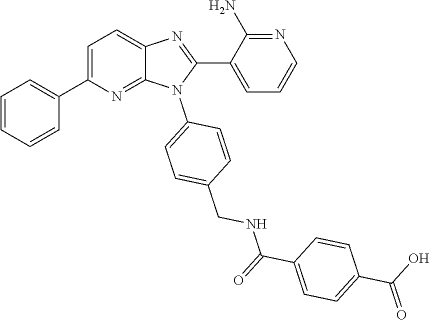 [SMILES: Nc1ncccc1-c1nc2ccc(-c3ccccc3)nc2n1-c1ccc(CNC(=O)c2ccc(C(=O)O)cc2)cc1] |
| 15 | 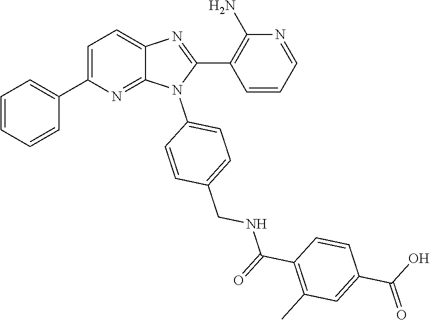 [SMILES: Cc1cc(C(=O)O)ccc1C(=O)NCc1ccc(-n2c(-c3cccnc3N)nc3ccc(-c4ccccc4)nc32)cc1] |
| 16 | 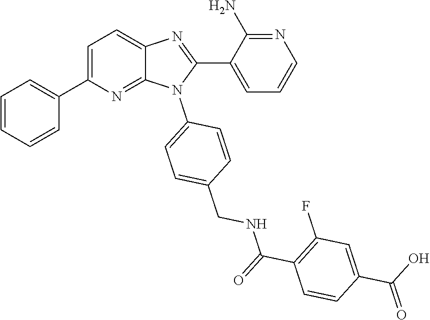 [SMILES: Nc1ncccc1-c1nc2ccc(-c3ccccc3)nc2n1-c1ccc(CNC(=O)c2ccc(C(=O)O)cc2F)cc1] |
| 17 | 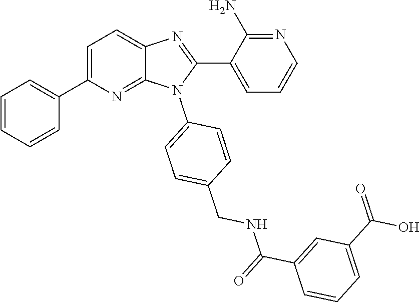 [SMILES: Nc1ncccc1-c1nc2ccc(-c3ccccc3)nc2n1-c1ccc(CNC(=O)c2cccc(C(=O)O)c2)cc1] |
| 18 | 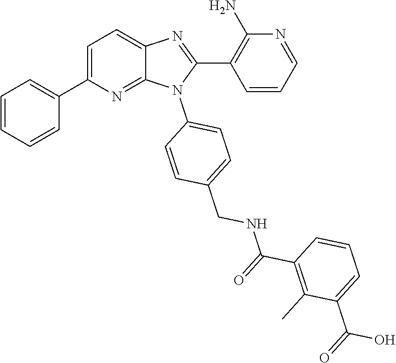 [SMILES: Cc1c(C(=O)O)cccc1C(=O)NCc1ccc(-n2c(-c3cccnc3N)nc3ccc(-c4ccccc4)nc32)cc1] |
| 19 | 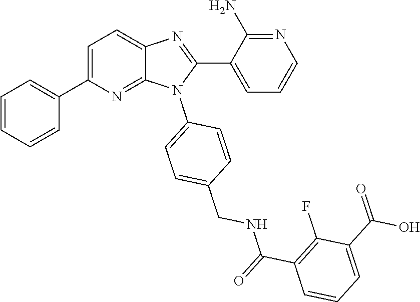 [SMILES: Nc1ncccc1-c1nc2ccc(-c3ccccc3)nc2n1-c1ccc(CNC(=O)c2cccc(C(=O)O)c2F)cc1] |
| 20 | 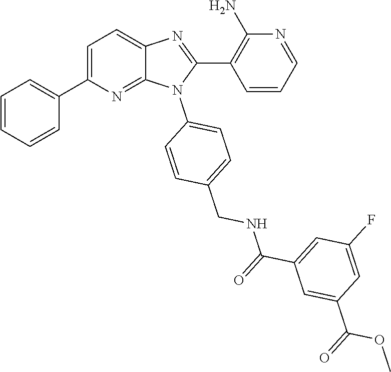 [SMILES: COC(=O)c1cc(F)cc(C(=O)NCc2ccc(-n3c(-c4cccnc4N)nc4ccc(-c5ccccc5)nc43)cc2)c1] |
| 21 | 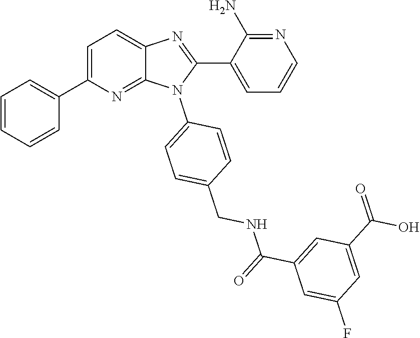 [SMILES: Nc1ncccc1-c1nc2ccc(-c3ccccc3)nc2n1-c1ccc(CNC(=O)c2cc(F)cc(C(=O)O)c2)cc1] |
| 22 | 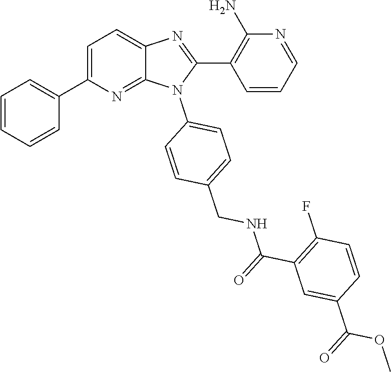 [SMILES: COC(=O)c1ccc(F)c(C(=O)NCc2ccc(-n3c(-c4cccnc4N)nc4ccc(-c5ccccc5)nc43)cc2)c1] |
| 23 | 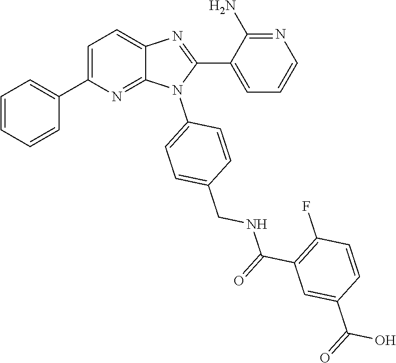 [SMILES: Nc1ncccc1-c1nc2ccc(-c3ccccc3)nc2n1-c1ccc(CNC(=O)c2cc(C(=O)O)ccc2F)cc1] |
| 24 | 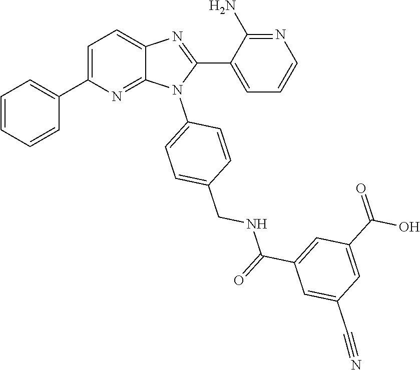 [SMILES: N#Cc1cc(C(=O)O)cc(C(=O)NCc2ccc(-n3c(-c4cccnc4N)nc4ccc(-c5ccccc5)nc43)cc2)c1] |
| 25 | 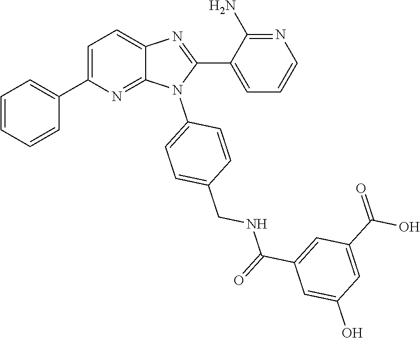 [SMILES: Nc1ncccc1-c1nc2ccc(-c3ccccc3)nc2n1-c1ccc(CNC(=O)c2cc(O)cc(C(=O)O)c2)cc1] |
| 26 | 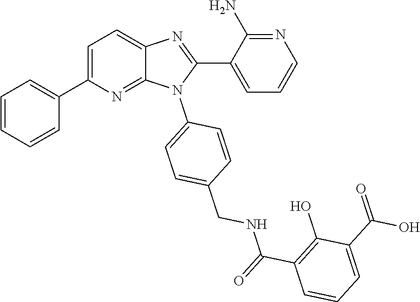 [SMILES: Nc1ncccc1-c1nc2ccc(-c3ccccc3)nc2n1-c1ccc(CNC(=O)c2cccc(C(=O)O)c2O)cc1] |
| 27 | 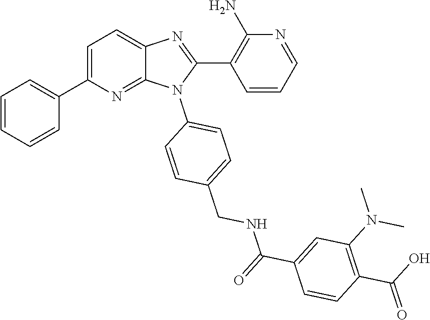 [SMILES: CN(C)c1cc(C(=O)NCc2ccc(-n3c(-c4cccnc4N)nc4ccc(-c5ccccc5)nc43)cc2)ccc1C(=O)O] |
| 28 | 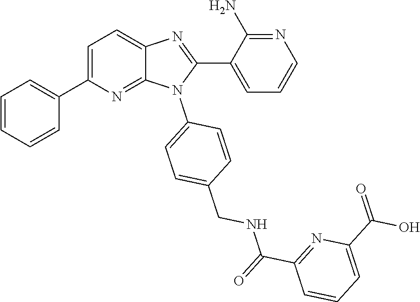 [SMILES: Nc1ncccc1-c1nc2ccc(-c3ccccc3)nc2n1-c1ccc(CNC(=O)c2cccc(C(=O)O)n2)cc1] |
| 29 | 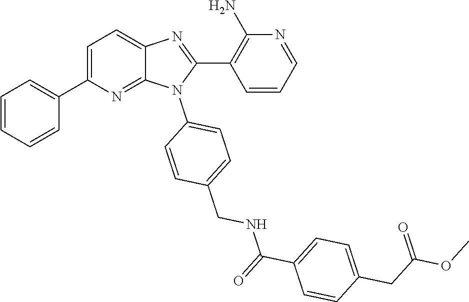 [SMILES: COC(=O)Cc1ccc(C(=O)NCc2ccc(-n3c(-c4cccnc4N)nc4ccc(-c5ccccc5)nc43)cc2)cc1] |
| 30 | 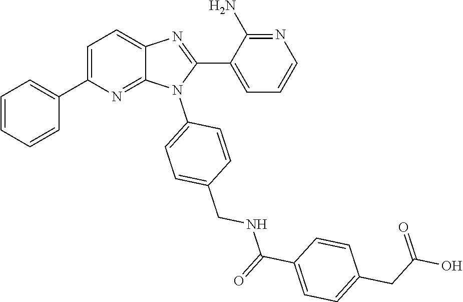 [SMILES: Nc1ncccc1-c1nc2ccc(-c3ccccc3)nc2n1-c1ccc(CNC(=O)c2ccc(CC(=O)O)cc2)cc1] |
| 31 | 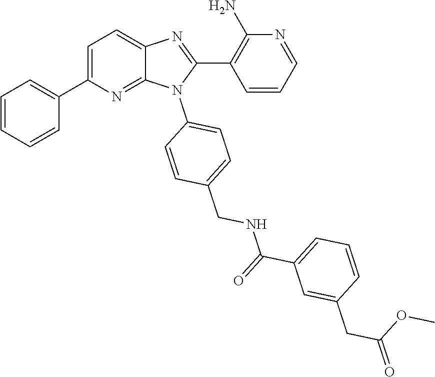 [SMILES: COC(=O)Cc1cccc(C(=O)NCc2ccc(-n3c(-c4cccnc4N)nc4ccc(-c5ccccc5)nc43)cc2)c1] |
| 32 | 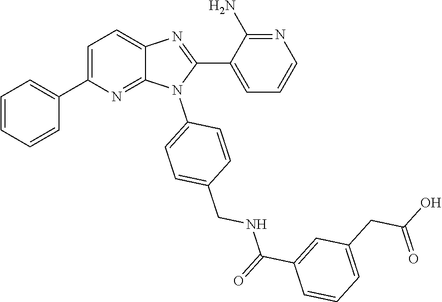 [SMILES: Nc1ncccc1-c1nc2ccc(-c3ccccc3)nc2n1-c1ccc(CNC(=O)c2cccc(CC(=O)O)c2)cc1] |
| 33 | 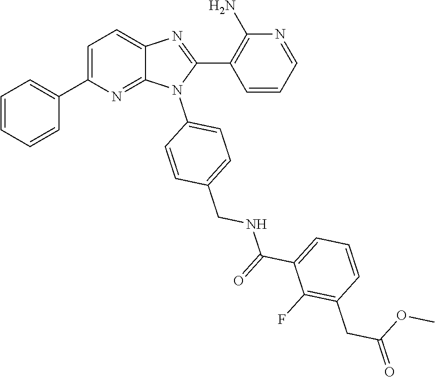 [SMILES: COC(=O)Cc1cccc(C(=O)NCc2ccc(-n3c(-c4cccnc4N)nc4ccc(-c5ccccc5)nc43)cc2)c1F] |
| 34 | 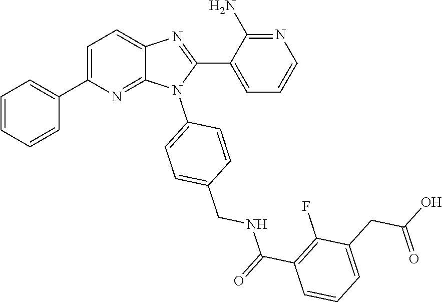 [SMILES: Nc1ncccc1-c1nc2ccc(-c3ccccc3)nc2n1-c1ccc(CNC(=O)c2cccc(CC(=O)O)c2F)cc1] |
| 35 | 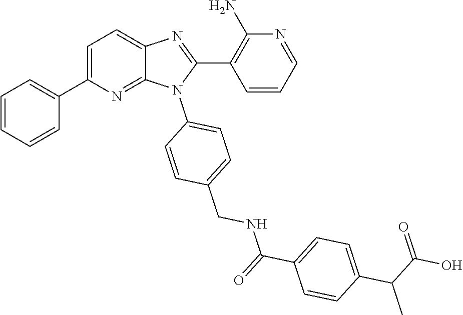 [SMILES: CC(C(=O)O)c1ccc(C(=O)NCc2ccc(-n3c(-c4cccnc4N)nc4ccc(-c5ccccc5)nc43)cc2)cc1] |
| 36 | 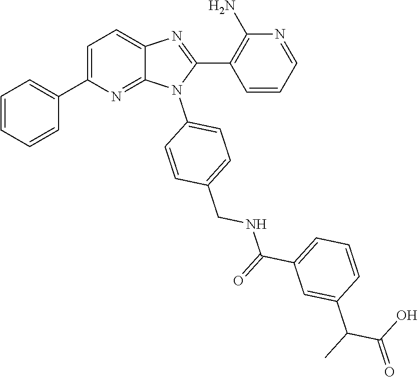 [SMILES: CC(C(=O)O)c1cccc(C(=O)NCc2ccc(-n3c(-c4cccnc4N)nc4ccc(-c5ccccc5)nc43)cc2)c1] |
| 37 | 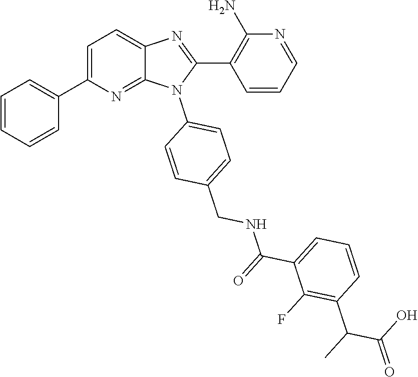 [SMILES: CC(C(=O)O)c1cccc(C(=O)NCc2ccc(-n3c(-c4cccnc4N)nc4ccc(-c5ccccc5)nc43)cc2)c1F] |
| 38 | 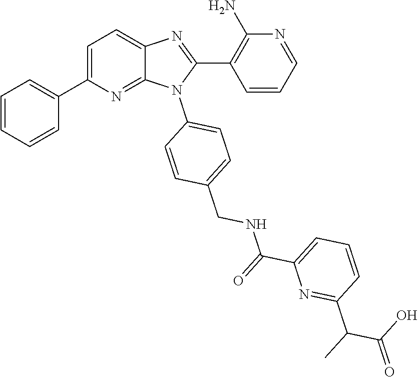 [SMILES: CC(C(=O)O)c1cccc(C(=O)NCc2ccc(-n3c(-c4cccnc4N)nc4ccc(-c5ccccc5)nc43)cc2)n1] |
| 39 | 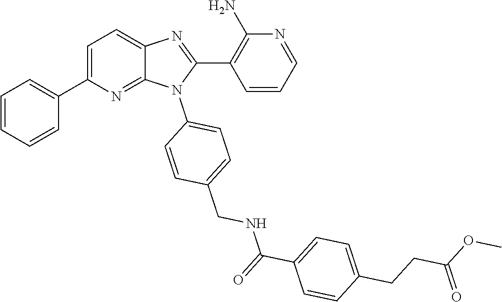 [SMILES: COC(=O)CCc1ccc(C(=O)NCc2ccc(-n3c(-c4cccnc4N)nc4ccc(-c5ccccc5)nc43)cc2)cc1] |
| 40 | 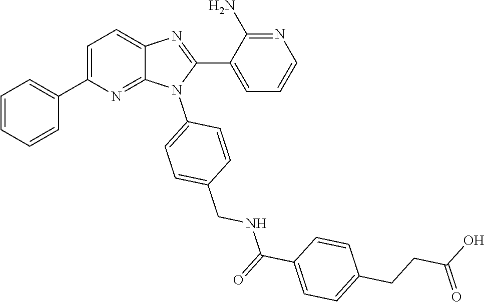 [SMILES: Nc1ncccc1-c1nc2ccc(-c3ccccc3)nc2n1-c1ccc(CNC(=O)c2ccc(CCC(=O)O)cc2)cc1] |
| 41 | 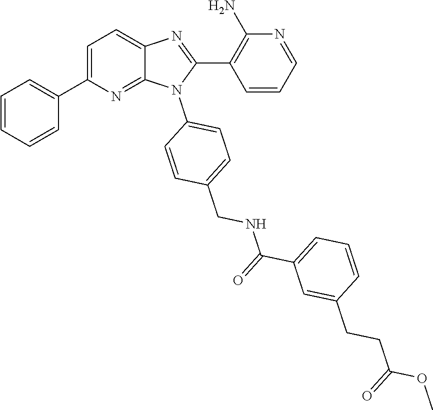 [SMILES: COC(=O)CCc1cccc(C(=O)NCc2ccc(-n3c(-c4cccnc4N)nc4ccc(-c5ccccc5)nc43)cc2)c1] |
| 42 | 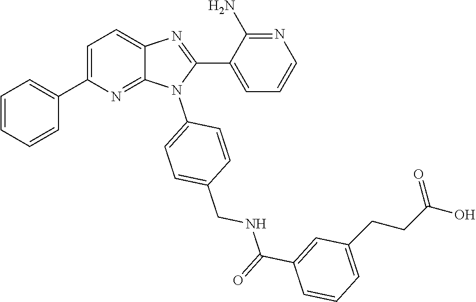 [SMILES: Nc1ncccc1-c1nc2ccc(-c3ccccc3)nc2n1-c1ccc(CNC(=O)c2cccc(CCC(=O)O)c2)cc1] |
| 43 | 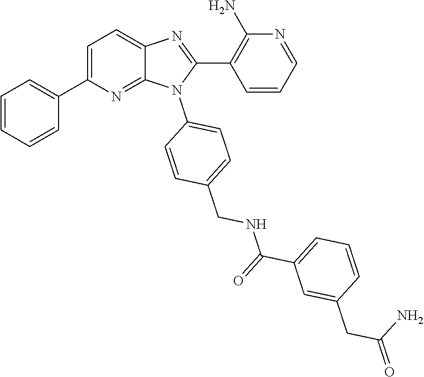 [SMILES: NC(=O)Cc1cccc(C(=O)NCc2ccc(-n3c(-c4cccnc4N)nc4ccc(-c5ccccc5)nc43)cc2)c1] |
| 44 | 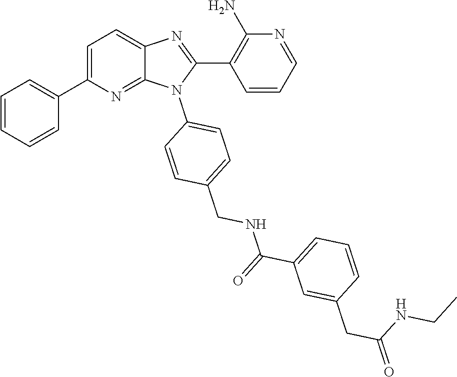 [SMILES: CCNC(=O)Cc1cccc(C(=O)NCc2ccc(-n3c(-c4cccnc4N)nc4ccc(-c5ccccc5)nc43)cc2)c1] |
| 45 | 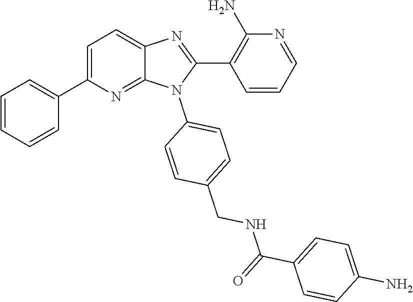 [SMILES: Nc1ccc(C(=O)NCc2ccc(-n3c(-c4cccnc4N)nc4ccc(-c5ccccc5)nc43)cc2)cc1] |
| 46 | 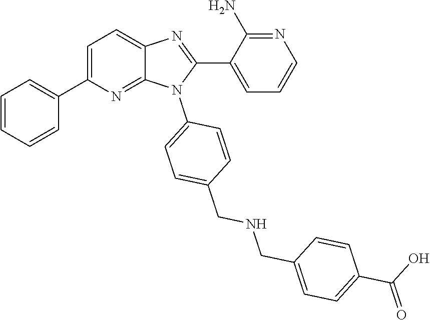 [SMILES: Nc1ncccc1-c1nc2ccc(-c3ccccc3)nc2n1-c1ccc(CNCc2ccc(C(=O)O)cc2)cc1] |
| 47 | 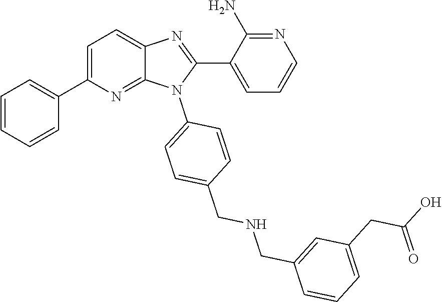 [SMILES: Nc1ncccc1-c1nc2ccc(-c3ccccc3)nc2n1-c1ccc(CNCc2cccc(CC(=O)O)c2)cc1] |
| 48 | 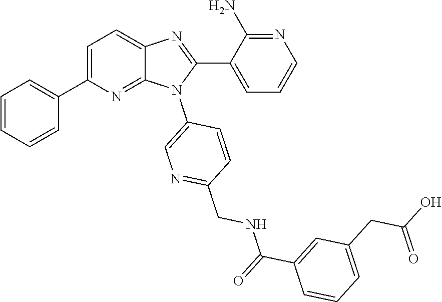 [SMILES: Nc1ncccc1-c1nc2ccc(-c3ccccc3)nc2n1-c1ccc(CNC(=O)c2cccc(CC(=O)O)c2)nc1] |
| 49 | 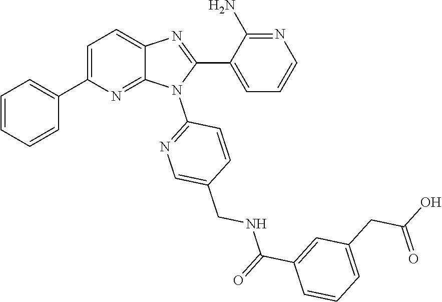 [SMILES: Nc1ncccc1-c1nc2ccc(-c3ccccc3)nc2n1-c1ccc(CNC(=O)c2cccc(CC(=O)O)c2)cn1] |
| 50 | 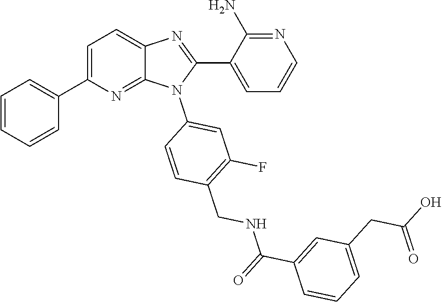 [SMILES: Nc1ncccc1-c1nc2ccc(-c3ccccc3)nc2n1-c1ccc(CNC(=O)c2cccc(CC(=O)O)c2)c(F)c1] |
| 51 | 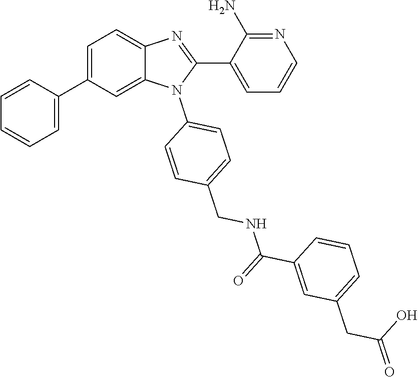 [SMILES: Nc1ncccc1-c1nc2ccc(-c3ccccc3)cc2n1-c1ccc(CNC(=O)c2cccc(CC(=O)O)c2)cc1] |
| 52 | 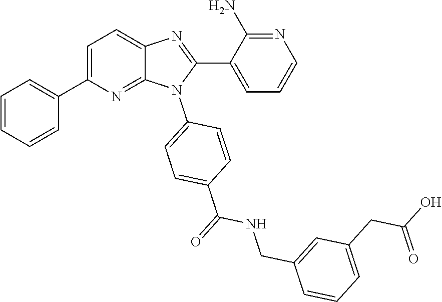 [SMILES: Nc1ncccc1-c1nc2ccc(-c3ccccc3)nc2n1-c1ccc(C(=O)NCc2cccc(CC(=O)O)c2)cc1] |
| 53 | 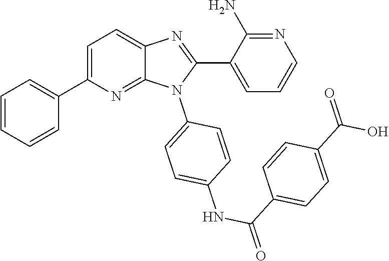 [SMILES: Nc1ncccc1-c1nc2ccc(-c3ccccc3)nc2n1-c1ccc(NC(=O)c2ccc(C(=O)O)cc2)cc1] |
| 54 | 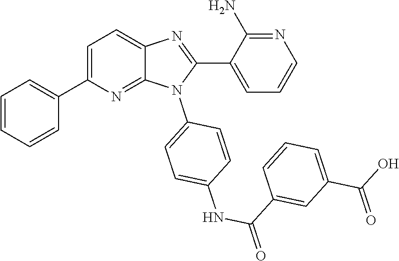 [SMILES: Nc1ncccc1-c1nc2ccc(-c3ccccc3)nc2n1-c1ccc(NC(=O)c2cccc(C(=O)O)c2)cc1] |
| 55 | 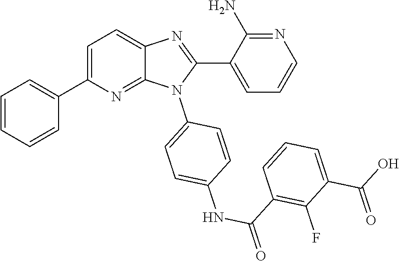 [SMILES: Nc1ncccc1-c1nc2ccc(-c3ccccc3)nc2n1-c1ccc(NC(=O)c2cccc(C(=O)O)c2F)cc1] |
| 56 | 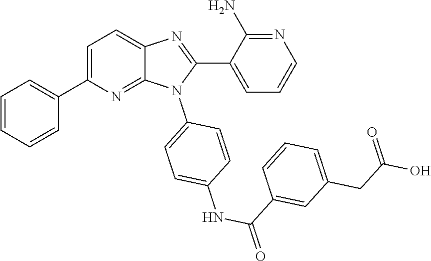 [SMILES: Nc1ncccc1-c1nc2ccc(-c3ccccc3)nc2n1-c1ccc(NC(=O)c2cccc(CC(=O)O)c2)cc1] |
| 57 | 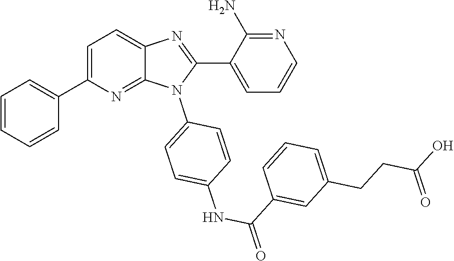 [SMILES: Nc1ncccc1-c1nc2ccc(-c3ccccc3)nc2n1-c1ccc(NC(=O)c2cccc(CCC(=O)O)c2)cc1] |
| 58 | 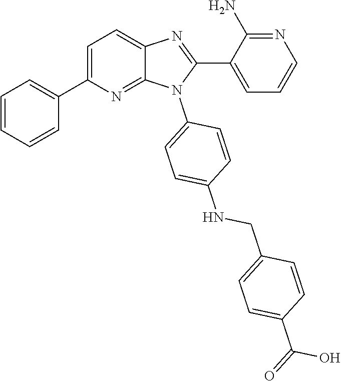 [SMILES: Nc1ncccc1-c1nc2ccc(-c3ccccc3)nc2n1-c1ccc(NCc2ccc(C(=O)O)cc2)cc1] |
| 59 | 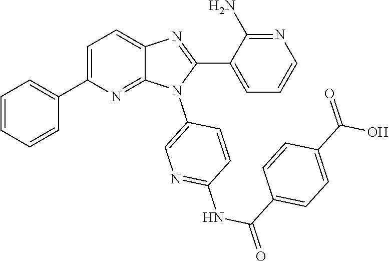 [SMILES: Nc1ncccc1-c1nc2ccc(-c3ccccc3)nc2n1-c1ccc(NC(=O)c2ccc(C(=O)O)cc2)nc1] |
| 60 | 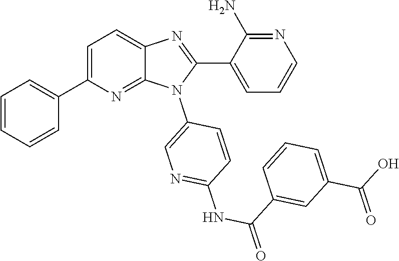 [SMILES: Nc1ncccc1-c1nc2ccc(-c3ccccc3)nc2n1-c1ccc(NC(=O)c2cccc(C(=O)O)c2)nc1] |
| 61 | 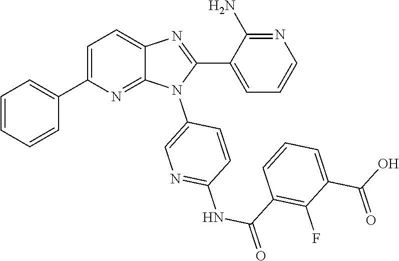 [SMILES: Nc1ncccc1-c1nc2ccc(-c3ccccc3)nc2n1-c1ccc(NC(=O)c2cccc(C(=O)O)c2F)nc1] |
| 62 | 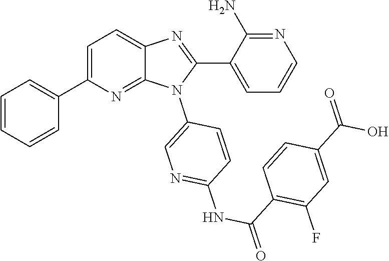 [SMILES: Nc1ncccc1-c1nc2ccc(-c3ccccc3)nc2n1-c1ccc(NC(=O)c2ccc(C(=O)O)cc2F)nc1] |
| 63 | 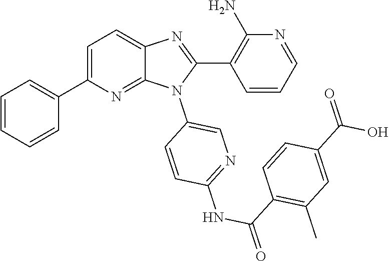 [SMILES: Cc1cc(C(=O)O)ccc1C(=O)Nc1ccc(-n2c(-c3cccnc3N)nc3ccc(-c4ccccc4)nc32)cn1] |
| 64 | 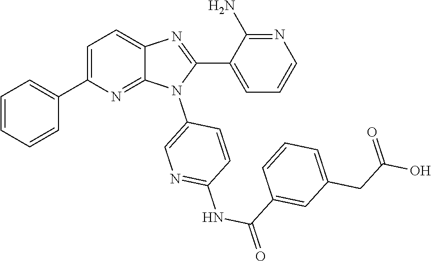 [SMILES: Nc1ncccc1-c1nc2ccc(-c3ccccc3)nc2n1-c1ccc(NC(=O)c2cccc(CC(=O)O)c2)nc1] |
| 65 | 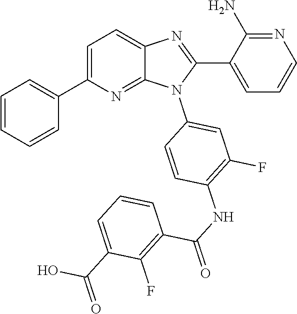 [SMILES: Nc1ncccc1-c1nc2ccc(-c3ccccc3)nc2n1-c1ccc(NC(=O)c2cccc(C(=O)O)c2F)c(F)c1] |
| 66 | 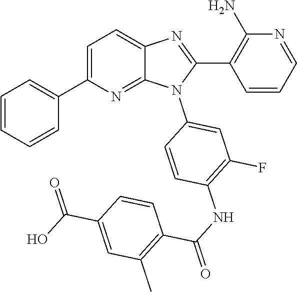 [SMILES: Cc1cc(C(=O)O)ccc1C(=O)Nc1ccc(-n2c(-c3cccnc3N)nc3ccc(-c4ccccc4)nc32)cc1F] |
| 67 | 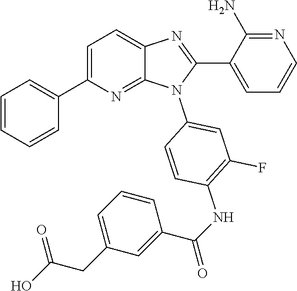 [SMILES: Nc1ncccc1-c1nc2ccc(-c3ccccc3)nc2n1-c1ccc(NC(=O)c2cccc(CC(=O)O)c2)c(F)c1] |
| 68 | 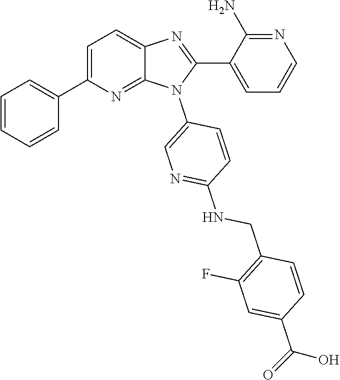 [SMILES: Nc1ncccc1-c1nc2ccc(-c3ccccc3)nc2n1-c1ccc(NCc2ccc(C(=O)O)cc2F)nc1] |
| 69 | 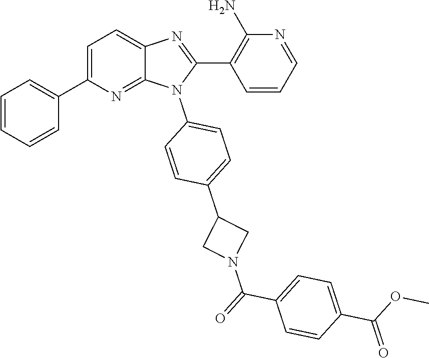 [SMILES: COC(=O)c1ccc(C(=O)N2CC(c3ccc(-n4c(-c5cccnc5N)nc5ccc(-c6ccccc6)nc54)cc3)C2)cc1] |
| 70 | 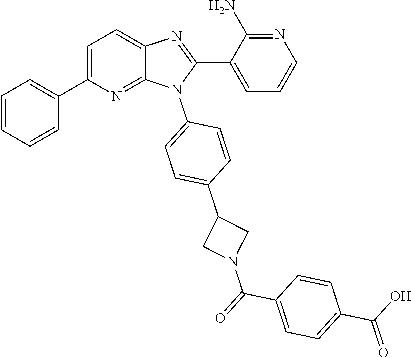 [SMILES: Nc1ncccc1-c1nc2ccc(-c3ccccc3)nc2n1-c1ccc(C2CN(C(=O)c3ccc(C(=O)O)cc3)C2)cc1] |
| 71 | 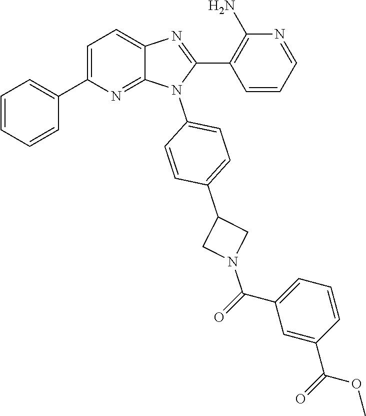 [SMILES: COC(=O)c1cccc(C(=O)N2CC(c3ccc(-n4c(-c5cccnc5N)nc5ccc(-c6ccccc6)nc54)cc3)C2)c1] |
| 72 | 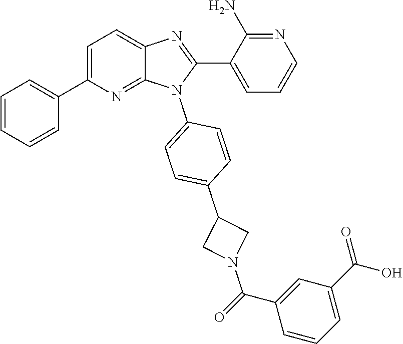 [SMILES: Nc1ncccc1-c1nc2ccc(-c3ccccc3)nc2n1-c1ccc(C2CN(C(=O)c3cccc(C(=O)O)c3)C2)cc1] |
| 73 | 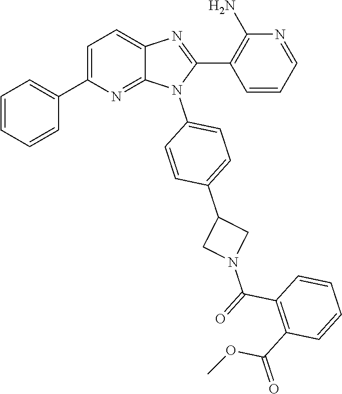 [SMILES: COC(=O)c1ccccc1C(=O)N1CC(c2ccc(-n3c(-c4cccnc4N)nc4ccc(-c5ccccc5)nc43)cc2)C1] |
| 74 | 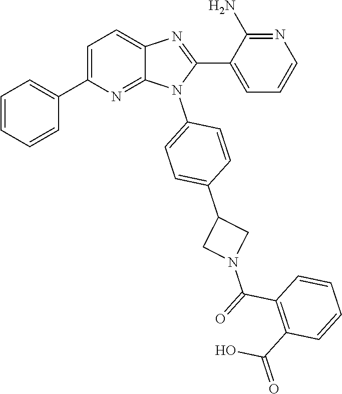 [SMILES: Nc1ncccc1-c1nc2ccc(-c3ccccc3)nc2n1-c1ccc(C2CN(C(=O)c3ccccc3C(=O)O)C2)cc1] |
| 75 | 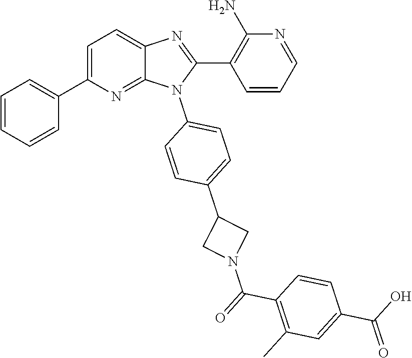 [SMILES: Cc1cc(C(=O)O)ccc1C(=O)N1CC(c2ccc(-n3c(-c4cccnc4N)nc4ccc(-c5ccccc5)nc43)cc2)C1] |
| 76 | 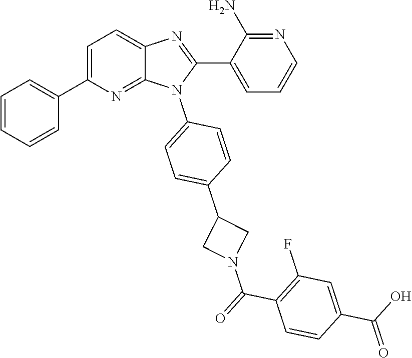 [SMILES: Nc1ncccc1-c1nc2ccc(-c3ccccc3)nc2n1-c1ccc(C2CN(C(=O)c3ccc(C(=O)O)cc3F)C2)cc1] |
| 77 | 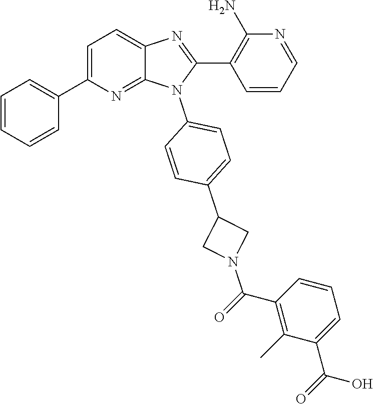 [SMILES: Cc1c(C(=O)O)cccc1C(=O)N1CC(c2ccc(-n3c(-c4cccnc4N)nc4ccc(-c5ccccc5)nc43)cc2)C1] |
| 78 | 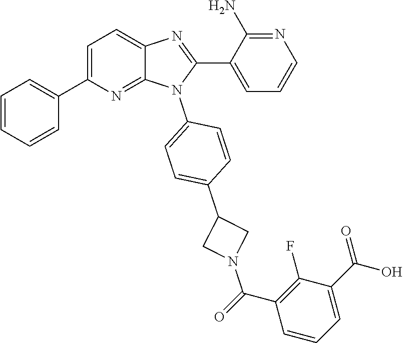 [SMILES: Nc1ncccc1-c1nc2ccc(-c3ccccc3)nc2n1-c1ccc(C2CN(C(=O)c3cccc(C(=O)O)c3F)C2)cc1] |
| 79 | 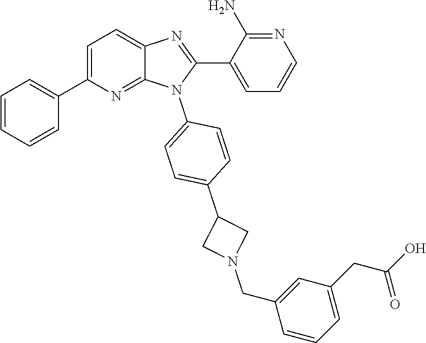 [SMILES: Nc1ncccc1-c1nc2ccc(-c3ccccc3)nc2n1-c1ccc(C2CN(Cc3cccc(CC(=O)O)c3)C2)cc1] |
| 80 | 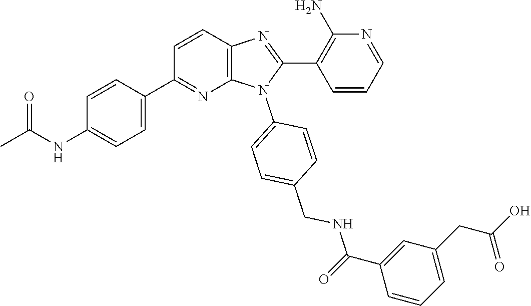 [SMILES: CC(=O)Nc1ccc(-c2ccc3nc(-c4cccnc4N)n(-c4ccc(CNC(=O)c5cccc(CC(=O)O)c5)cc4)c3n2)cc1] |
| 81 | 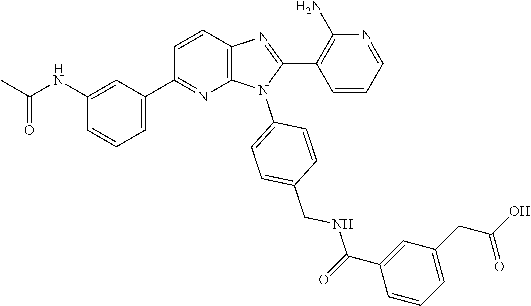 [SMILES: CC(=O)Nc1cccc(-c2ccc3nc(-c4cccnc4N)n(-c4ccc(CNC(=O)c5cccc(CC(=O)O)c5)cc4)c3n2)c1] |
| 82 | 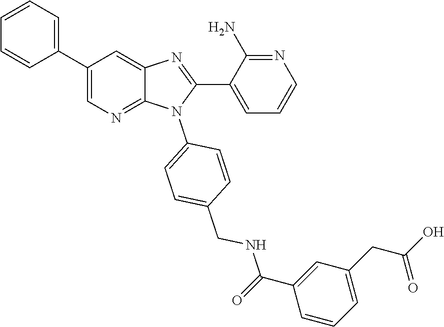 [SMILES: Nc1ncccc1-c1nc2cc(-c3ccccc3)cnc2n1-c1ccc(CNC(=O)c2cccc(CC(=O)O)c2)cc1] |
| 83 | 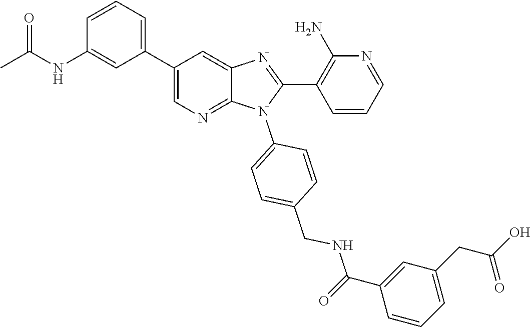 [SMILES: CC(=O)Nc1cccc(-c2cnc3c(c2)nc(-c2cccnc2N)n3-c2ccc(CNC(=O)c3cccc(CC(=O)O)c3)cc2)c1] |
| 84 | 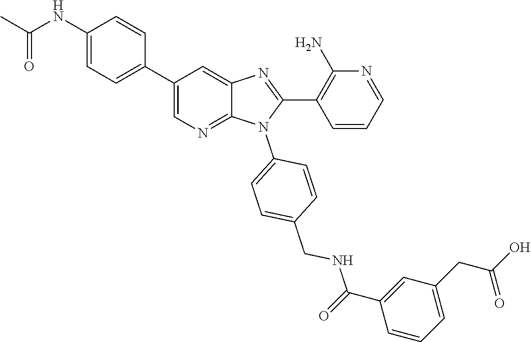 [SMILES: CC(=O)Nc1ccc(-c2cnc3c(c2)nc(-c2cccnc2N)n3-c2ccc(CNC(=O)c3cccc(CC(=O)O)c3)cc2)cc1] |
| 85 | 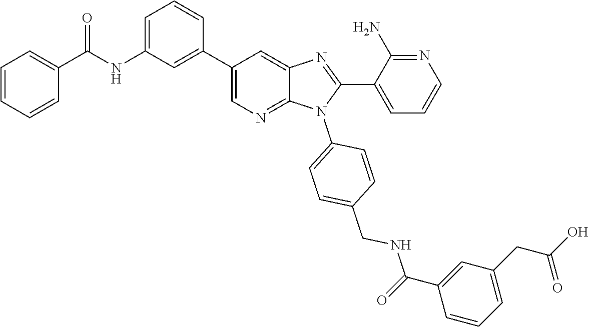 [SMILES: Nc1ncccc1-c1nc2cc(-c3cccc(NC(=O)c4ccccc4)c3)cnc2n1-c1ccc(CNC(=O)c2cccc(CC(=O)O)c2)cc1] |
| 86 | 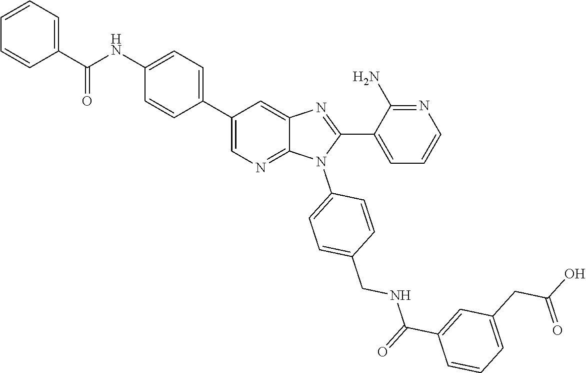 [SMILES: Nc1ncccc1-c1nc2cc(-c3ccc(NC(=O)c4ccccc4)cc3)cnc2n1-c1ccc(CNC(=O)c2cccc(CC(=O)O)c2)cc1] |
| 87 | 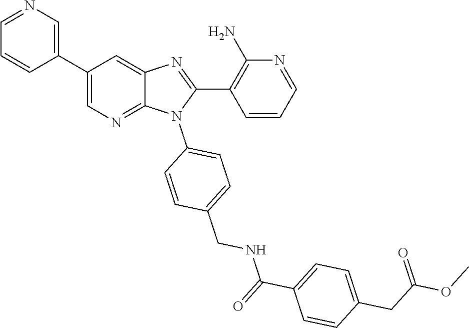 [SMILES: COC(=O)Cc1ccc(C(=O)NCc2ccc(-n3c(-c4cccnc4N)nc4cc(-c5cccnc5)cnc43)cc2)cc1] |
| 88 | 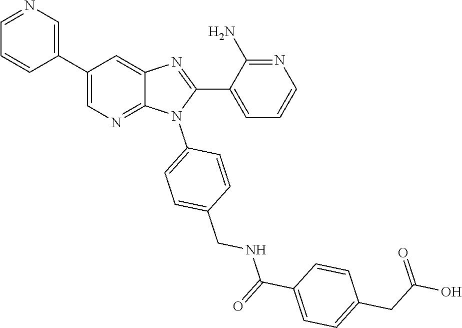 [SMILES: Nc1ncccc1-c1nc2cc(-c3cccnc3)cnc2n1-c1ccc(CNC(=O)c2ccc(CC(=O)O)cc2)cc1] |
| 89 | 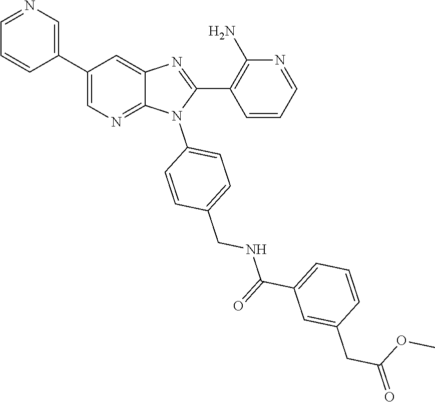 [SMILES: COC(=O)Cc1cccc(C(=O)NCc2ccc(-n3c(-c4cccnc4N)nc4cc(-c5cccnc5)cnc43)cc2)c1] |
| 90 | 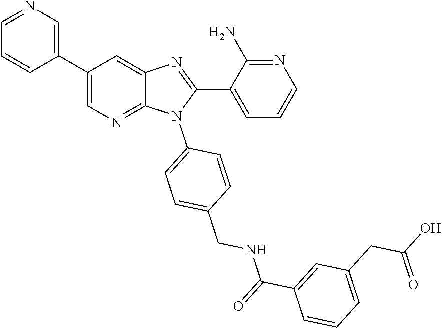 [SMILES: Nc1ncccc1-c1nc2cc(-c3cccnc3)cnc2n1-c1ccc(CNC(=O)c2cccc(CC(=O)O)c2)cc1] |
| 91 | 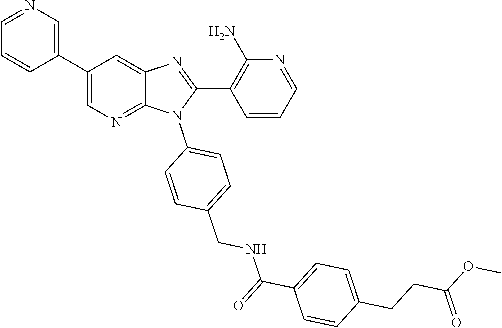 [SMILES: COC(=O)CCc1ccc(C(=O)NCc2ccc(-n3c(-c4cccnc4N)nc4cc(-c5cccnc5)cnc43)cc2)cc1] |
| 92 | 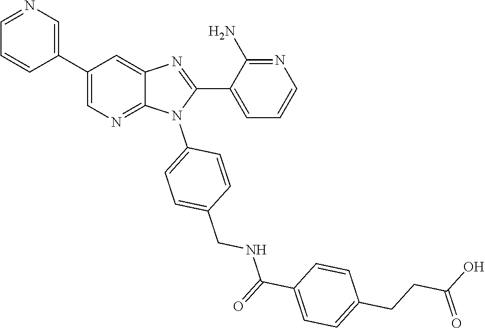 [SMILES: Nc1ncccc1-c1nc2cc(-c3cccnc3)cnc2n1-c1ccc(CNC(=O)c2ccc(CCC(=O)O)cc2)cc1] |
| 93 | 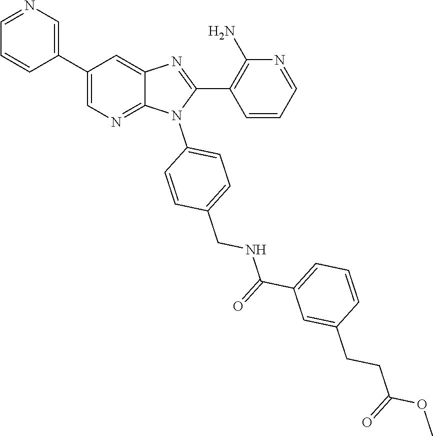 [SMILES: COC(=O)CCc1cccc(C(=O)NCc2ccc(-n3c(-c4cccnc4N)nc4cc(-c5cccnc5)cnc43)cc2)c1] |
| 94 | 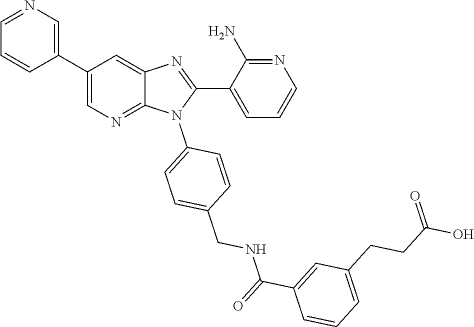 [SMILES: Nc1ncccc1-c1nc2cc(-c3cccnc3)cnc2n1-c1ccc(CNC(=O)c2cccc(CCC(=O)O)c2)cc1] |
| 95 | 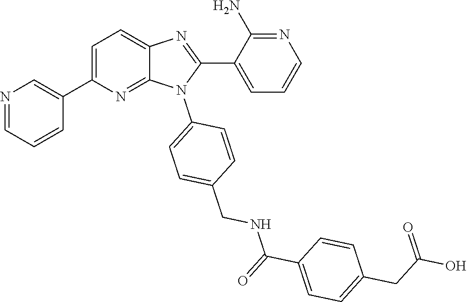 [SMILES: Nc1ncccc1-c1nc2ccc(-c3cccnc3)nc2n1-c1ccc(CNC(=O)c2ccc(CC(=O)O)cc2)cc1] |
| 96 | 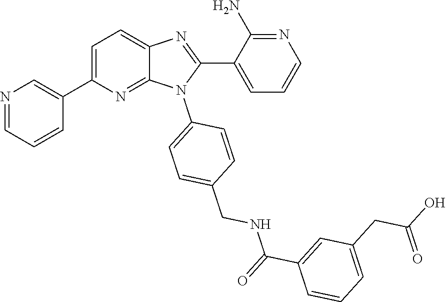 [SMILES: Nc1ncccc1-c1nc2ccc(-c3cccnc3)nc2n1-c1ccc(CNC(=O)c2cccc(CC(=O)O)c2)cc1] |
| 97 | 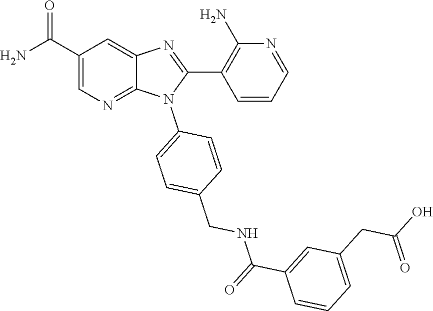 [SMILES: NC(=O)c1cnc2c(c1)nc(-c1cccnc1N)n2-c1ccc(CNC(=O)c2cccc(CC(=O)O)c2)cc1] |
| 98 | 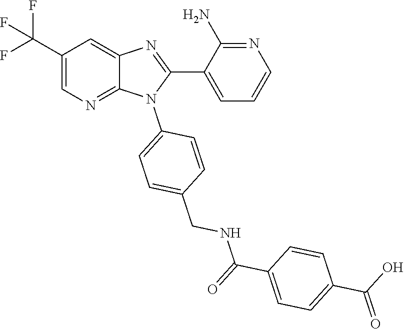 [SMILES: Nc1ncccc1-c1nc2cc(C(F)(F)F)cnc2n1-c1ccc(CNC(=O)c2ccc(C(=O)O)cc2)cc1] |
| 99 | 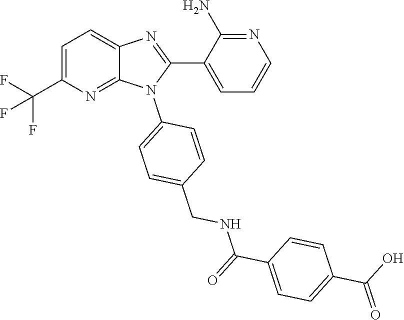 [SMILES: Nc1ncccc1-c1nc2ccc(C(F)(F)F)nc2n1-c1ccc(CNC(=O)c2ccc(C(=O)O)cc2)cc1] |
| 100 | 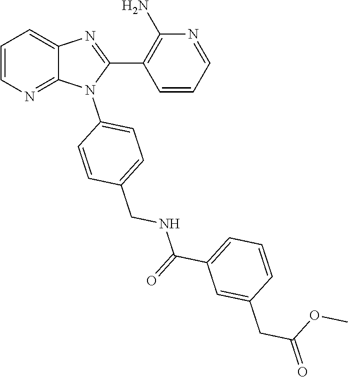 [SMILES: COC(=O)Cc1cccc(C(=O)NCc2ccc(-n3c(-c4cccnc4N)nc4cccnc43)cc2)c1] |
| 101 | 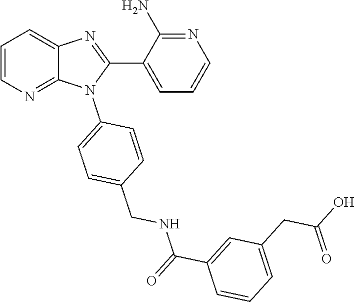 [SMILES: Nc1ncccc1-c1nc2cccnc2n1-c1ccc(CNC(=O)c2cccc(CC(=O)O)c2)cc1] |
| 102 | 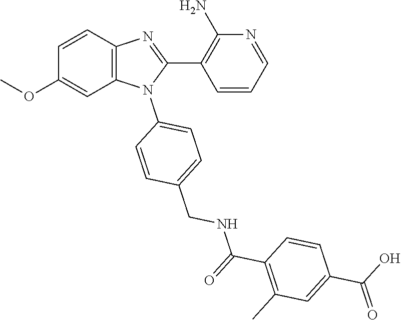 [SMILES: COc1ccc2nc(-c3cccnc3N)n(-c3ccc(CNC(=O)c4ccc(C(=O)O)cc4C)cc3)c2c1] |
| 103 | 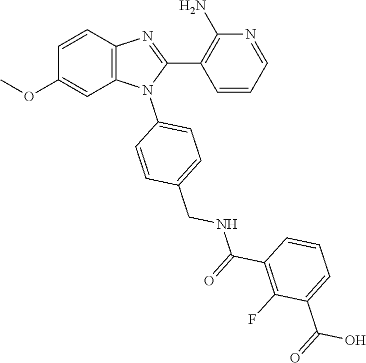 [SMILES: COc1ccc2nc(-c3cccnc3N)n(-c3ccc(CNC(=O)c4cccc(C(=O)O)c4F)cc3)c2c1] |
| 104 | 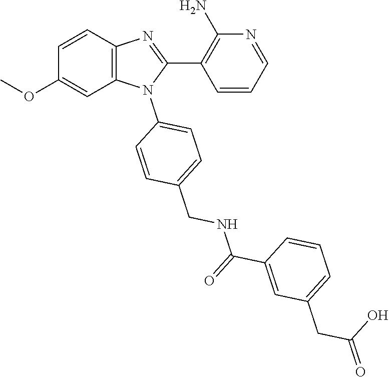 [SMILES: COc1ccc2nc(-c3cccnc3N)n(-c3ccc(CNC(=O)c4cccc(CC(=O)O)c4)cc3)c2c1] |
| 105 | 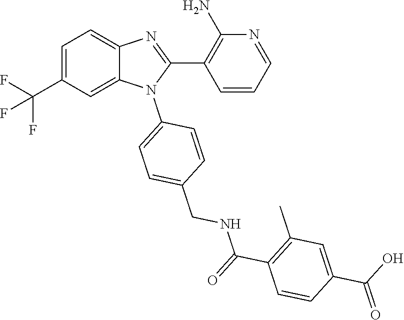 [SMILES: Cc1cc(C(=O)O)ccc1C(=O)NCc1ccc(-n2c(-c3cccnc3N)nc3ccc(C(F)(F)F)cc32)cc1] |
| 106 | 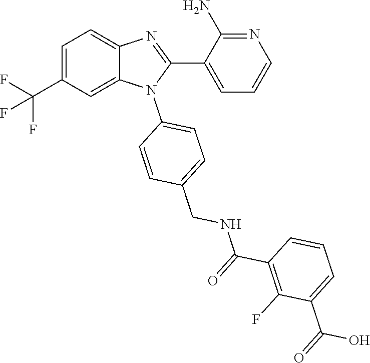 [SMILES: Nc1ncccc1-c1nc2ccc(C(F)(F)F)cc2n1-c1ccc(CNC(=O)c2cccc(C(=O)O)c2F)cc1] |
| 107 | 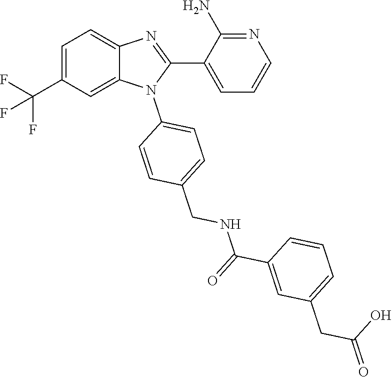 [SMILES: Nc1ncccc1-c1nc2ccc(C(F)(F)F)cc2n1-c1ccc(CNC(=O)c2cccc(CC(=O)O)c2)cc1] |
| 108 | 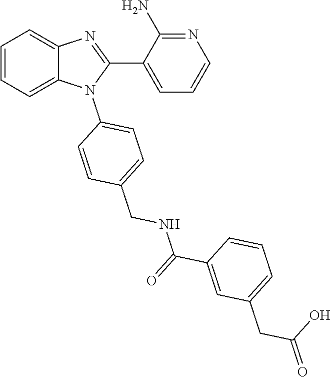 [SMILES: Nc1ncccc1-c1nc2ccccc2n1-c1ccc(CNC(=O)c2cccc(CC(=O)O)c2)cc1] |
| 109 | 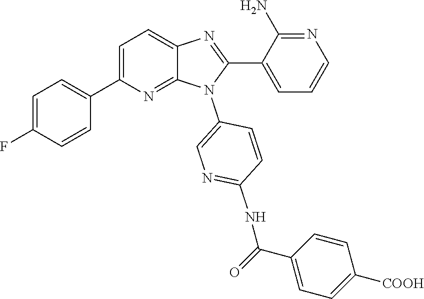 [SMILES: Nc1ncccc1-c1nc2ccc(-c3ccc(F)cc3)nc2n1-c1ccc(NC(=O)c2ccc(C(=O)O)cc2)nc1] |
| 110 | 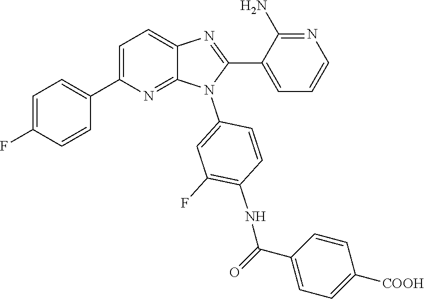 [SMILES: Nc1ncccc1-c1nc2ccc(-c3ccc(F)cc3)nc2n1-c1ccc(NC(=O)c2ccc(C(=O)O)cc2)c(F)c1] |
| 111 | 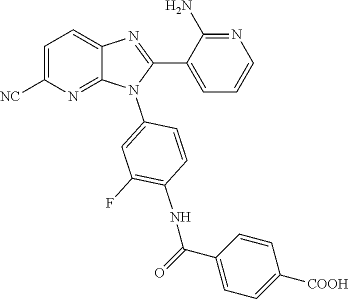 [SMILES: N#Cc1ccc2nc(-c3cccnc3N)n(-c3ccc(NC(=O)c4ccc(C(=O)O)cc4)c(F)c3)c2n1] |
| 112 | 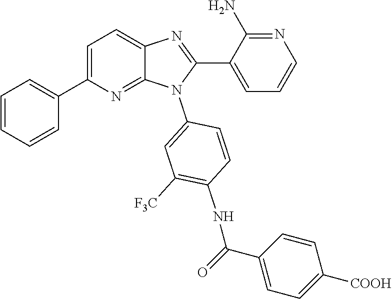 [SMILES: Nc1ncccc1-c1nc2ccc(-c3ccccc3)nc2n1-c1ccc(NC(=O)c2ccc(C(=O)O)cc2)c(C(F)(F)F)c1] |
| 113 | 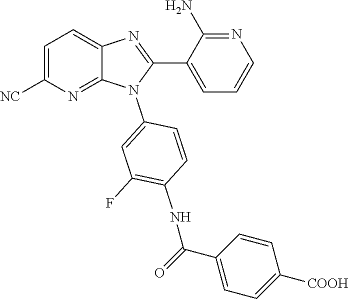 [SMILES: N#Cc1ccc2nc(-c3cccnc3N)n(-c3ccc(NC(=O)c4ccc(C(=O)O)cc4)c(F)c3)c2n1] |
| 114 | 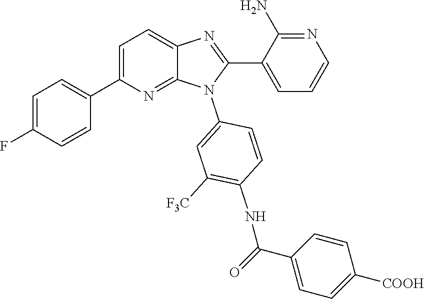 [SMILES: Nc1ncccc1-c1nc2ccc(-c3ccc(F)cc3)nc2n1-c1ccc(NC(=O)c2ccc(C(=O)O)cc2)c(C(F)(F)F)c1] |
| 115 | 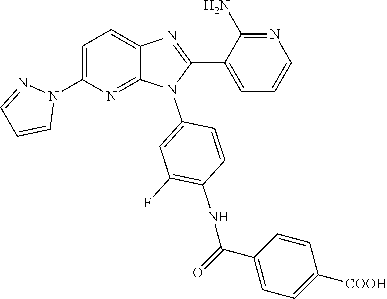 [SMILES: Nc1ncccc1-c1nc2ccc(-n3cccn3)nc2n1-c1ccc(NC(=O)c2ccc(C(=O)O)cc2)c(F)c1] |
| 116 | 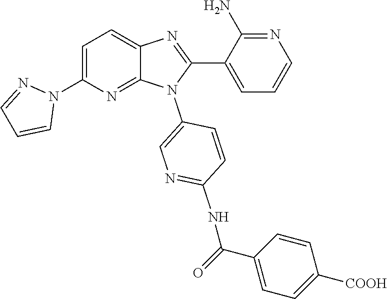 [SMILES: Nc1ncccc1-c1nc2ccc(-n3cccn3)nc2n1-c1ccc(NC(=O)c2ccc(C(=O)O)cc2)nc1] |
| 117 | 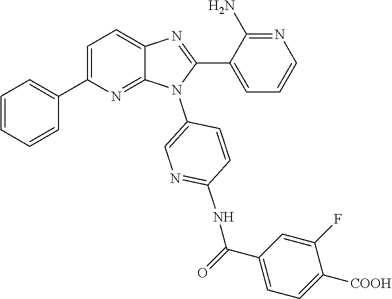 [SMILES: Nc1ncccc1-c1nc2ccc(-c3ccccc3)nc2n1-c1ccc(NC(=O)c2ccc(C(=O)O)c(F)c2)nc1] |
| 118 | 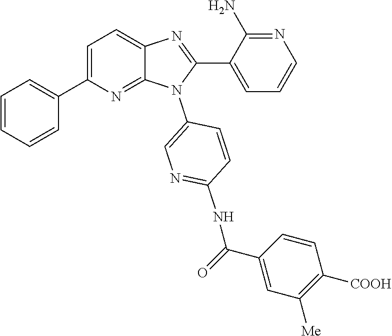 [SMILES: Cc1cc(C(=O)Nc2ccc(-n3c(-c4cccnc4N)nc4ccc(-c5ccccc5)nc43)cn2)ccc1C(=O)O] |
| 119 | 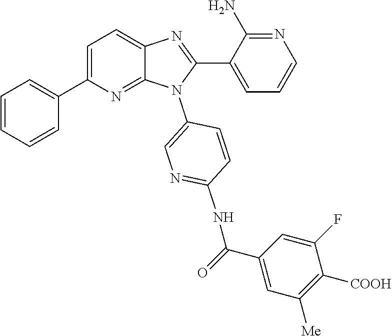 [SMILES: Cc1cc(C(=O)Nc2ccc(-n3c(-c4cccnc4N)nc4ccc(-c5ccccc5)nc43)cn2)cc(F)c1C(=O)O] |
| 120 | 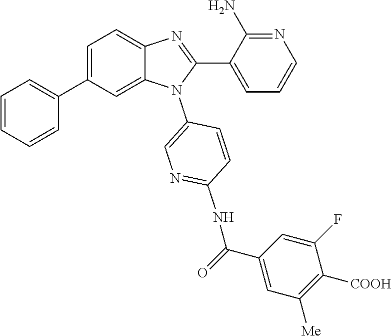 [SMILES: Cc1cc(C(=O)Nc2ccc(-n3c(-c4cccnc4N)nc4ccc(-c5ccccc5)cc43)cn2)cc(F)c1C(=O)O] |
| 121 | 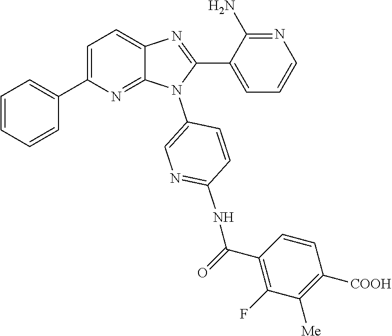 [SMILES: Cc1c(C(=O)O)ccc(C(=O)Nc2ccc(-n3c(-c4cccnc4N)nc4ccc(-c5ccccc5)nc43)cn2)c1F] |
| 122 | 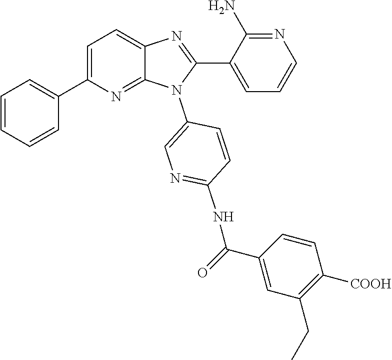 [SMILES: CCc1cc(C(=O)Nc2ccc(-n3c(-c4cccnc4N)nc4ccc(-c5ccccc5)nc43)cn2)ccc1C(=O)O] |
| 123 | 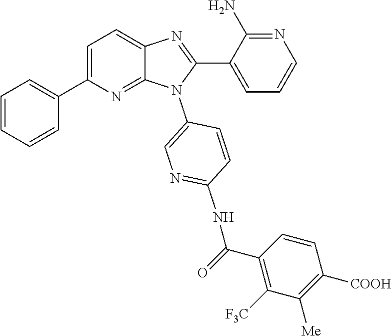 [SMILES: Cc1c(C(=O)O)ccc(C(=O)Nc2ccc(-n3c(-c4cccnc4N)nc4ccc(-c5ccccc5)nc43)cn2)c1C(F)(F)F] |
| 124 | 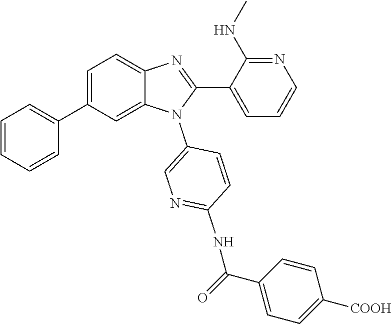 [SMILES: CNc1ncccc1-c1nc2ccc(-c3ccccc3)cc2n1-c1ccc(NC(=O)c2ccc(C(=O)O)cc2)nc1] |
| 125 | 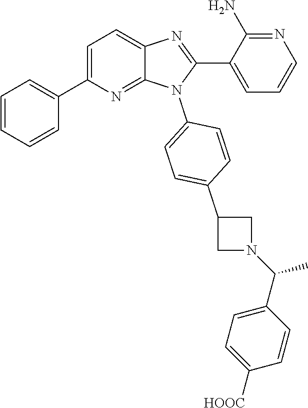 [SMILES: C[C@H](c1ccc(C(=O)O)cc1)N1CC(c2ccc(-n3c(-c4cccnc4N)nc4ccc(-c5ccccc5)nc43)cc2)C1] |
| 126 | 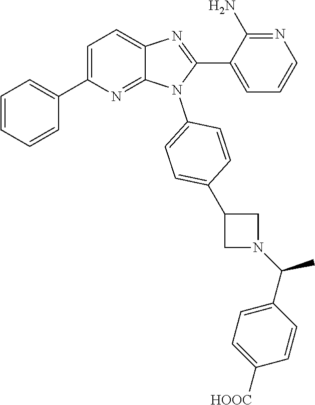 [SMILES: C[C@@H](c1ccc(C(=O)O)cc1)N1CC(c2ccc(-n3c(-c4cccnc4N)nc4ccc(-c5ccccc5)nc43)cc2)C1] |
| 127 | 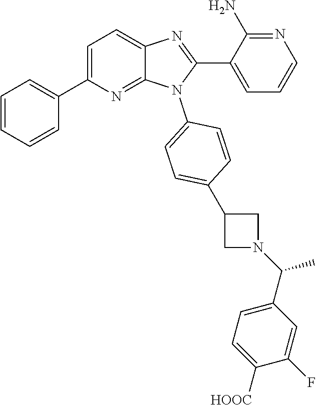 [SMILES: C[C@H](c1ccc(C(=O)O)c(F)c1)N1CC(c2ccc(-n3c(-c4cccnc4N)nc4ccc(-c5ccccc5)nc43)cc2)C1] |
| 128 | 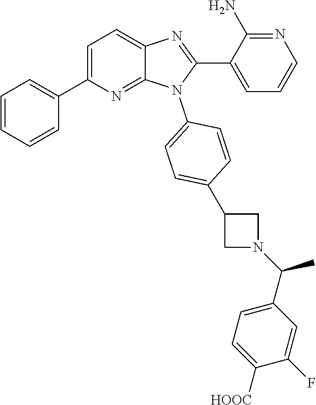 [SMILES: C[C@@H](c1ccc(C(=O)O)c(F)c1)N1CC(c2ccc(-n3c(-c4cccnc4N)nc4ccc(-c5ccccc5)nc43)cc2)C1] |
| 129 | 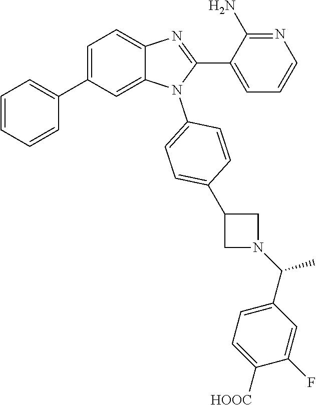 [SMILES: C[C@H](c1ccc(C(=O)O)c(F)c1)N1CC(c2ccc(-n3c(-c4cccnc4N)nc4ccc(-c5ccccc5)cc43)cc2)C1] |
| 130 | 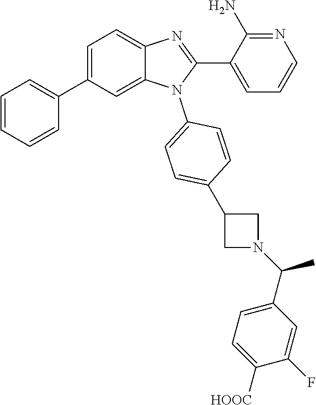 [SMILES: C[C@@H](c1ccc(C(=O)O)c(F)c1)N1CC(c2ccc(-n3c(-c4cccnc4N)nc4ccc(-c5ccccc5)cc43)cc2)C1] |
| 131 | 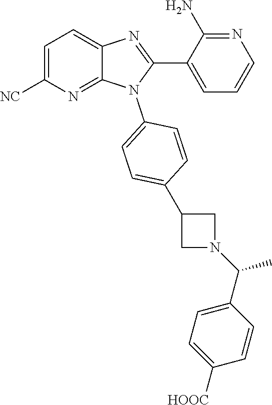 [SMILES: C[C@H](c1ccc(C(=O)O)cc1)N1CC(c2ccc(-n3c(-c4cccnc4N)nc4ccc(C#N)nc43)cc2)C1] |
| 132 | 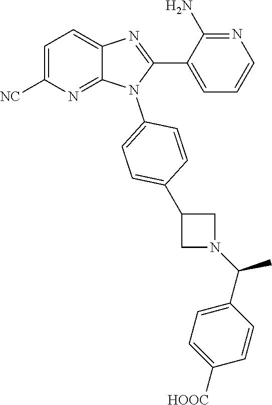 [SMILES: C[C@@H](c1ccc(C(=O)O)cc1)N1CC(c2ccc(-n3c(-c4cccnc4N)nc4ccc(C#N)nc43)cc2)C1] |
| 133 | 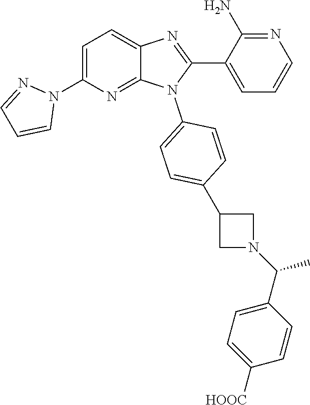 [SMILES: C[C@H](c1ccc(C(=O)O)cc1)N1CC(c2ccc(-n3c(-c4cccnc4N)nc4ccc(-n5cccn5)nc43)cc2)C1] |
| 134 | 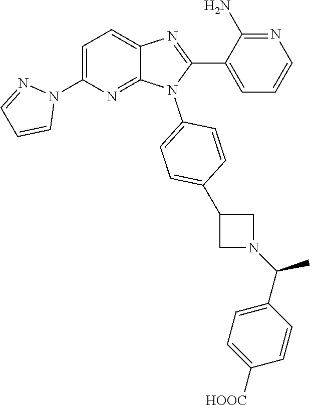 [SMILES: C[C@@H](c1ccc(C(=O)O)cc1)N1CC(c2ccc(-n3c(-c4cccnc4N)nc4ccc(-n5cccn5)nc43)cc2)C1] |
| 135 | 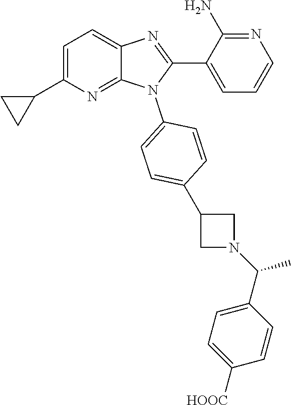 [SMILES: C[C@H](c1ccc(C(=O)O)cc1)N1CC(c2ccc(-n3c(-c4cccnc4N)nc4ccc(C5CC5)nc43)cc2)C1] |
| 136 | 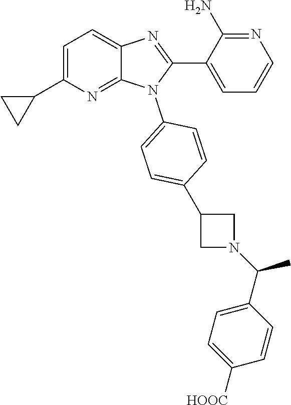 [SMILES: C[C@@H](c1ccc(C(=O)O)cc1)N1CC(c2ccc(-n3c(-c4cccnc4N)nc4ccc(C5CC5)nc43)cc2)C1] |
| 137 | 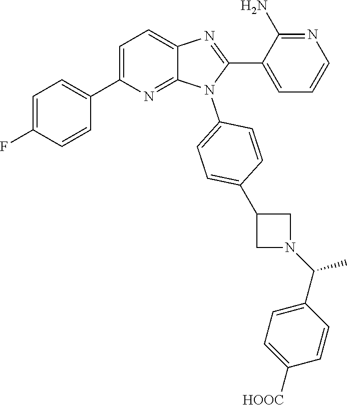 [SMILES: C[C@H](c1ccc(C(=O)O)cc1)N1CC(c2ccc(-n3c(-c4cccnc4N)nc4ccc(-c5ccc(F)cc5)nc43)cc2)C1] |
| 138 | 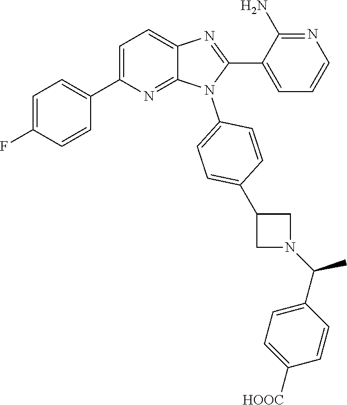 [SMILES: C[C@@H](c1ccc(C(=O)O)cc1)N1CC(c2ccc(-n3c(-c4cccnc4N)nc4ccc(-c5ccc(F)cc5)nc43)cc2)C1] |
| 139 | 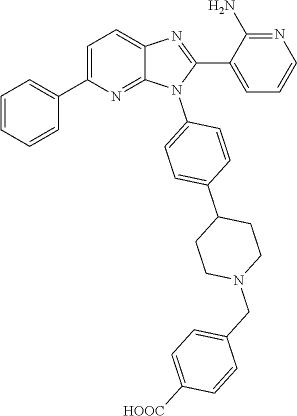 [SMILES: Nc1ncccc1-c1nc2ccc(-c3ccccc3)nc2n1-c1ccc(C2CCN(Cc3ccc(C(=O)O)cc3)CC2)cc1] |
| 140 | 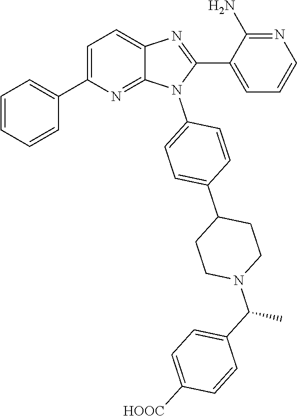 [SMILES: C[C@H](c1ccc(C(=O)O)cc1)N1CCC(c2ccc(-n3c(-c4cccnc4N)nc4ccc(-c5ccccc5)nc43)cc2)CC1] |
| 141 | 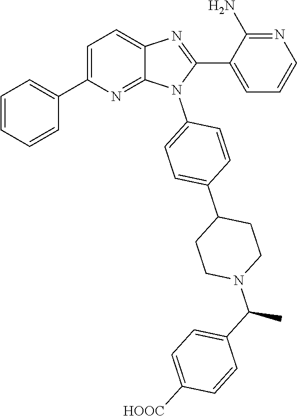 [SMILES: C[C@@H](c1ccc(C(=O)O)cc1)N1CCC(c2ccc(-n3c(-c4cccnc4N)nc4ccc(-c5ccccc5)nc43)cc2)CC1] |
| 142 | 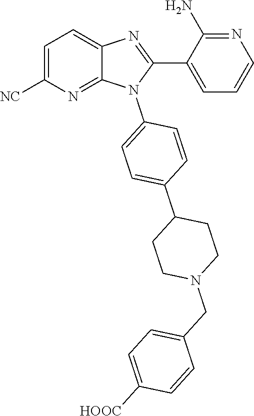 [SMILES: N#Cc1ccc2nc(-c3cccnc3N)n(-c3ccc(C4CCN(Cc5ccc(C(=O)O)cc5)CC4)cc3)c2n1] |
| 143 | 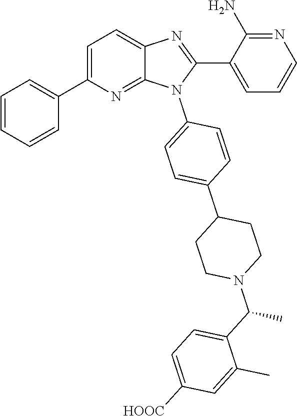 [SMILES: Cc1cc(C(=O)O)ccc1[C@@H](C)N1CCC(c2ccc(-n3c(-c4cccnc4N)nc4ccc(-c5ccccc5)nc43)cc2)CC1] |
| 144 | 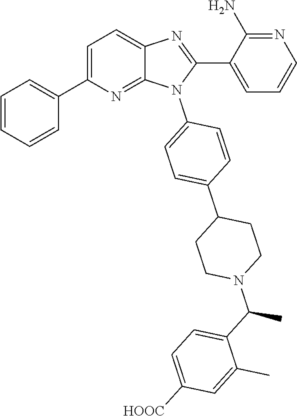 [SMILES: Cc1cc(C(=O)O)ccc1[C@H](C)N1CCC(c2ccc(-n3c(-c4cccnc4N)nc4ccc(-c5ccccc5)nc43)cc2)CC1] |
| 145 | 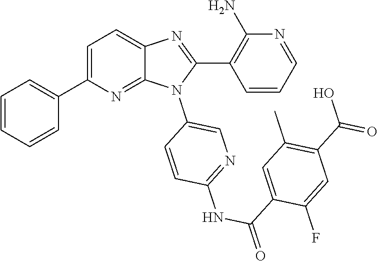 [SMILES: Cc1cc(C(=O)Nc2ccc(-n3c(-c4cccnc4N)nc4ccc(-c5ccccc5)nc43)cn2)c(F)cc1C(=O)O] |
| 146 | 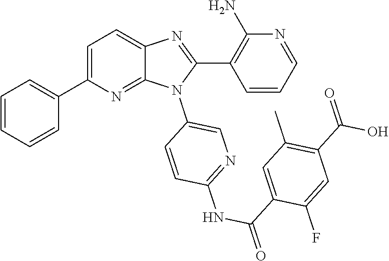 [SMILES: Cc1cc(C(=O)Nc2ccc(-n3c(-c4cccnc4N)nc4ccc(-c5ccccc5)nc43)cn2)c(F)cc1C(=O)O] |
| 147 | 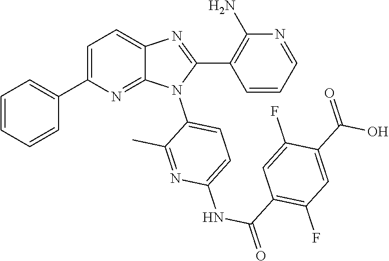 [SMILES: Cc1nc(NC(=O)c2cc(F)c(C(=O)O)cc2F)ccc1-n1c(-c2cccnc2N)nc2ccc(-c3ccccc3)nc21] |
| 148 | 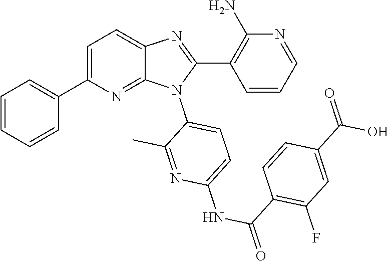 [SMILES: Cc1nc(NC(=O)c2ccc(C(=O)O)cc2F)ccc1-n1c(-c2cccnc2N)nc2ccc(-c3ccccc3)nc21] |
| 149 | 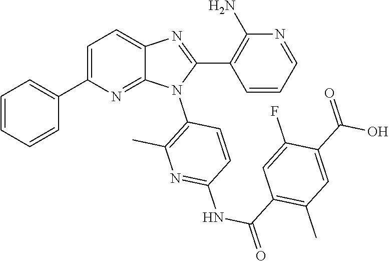 [SMILES: Cc1cc(C(=O)O)c(F)cc1C(=O)Nc1ccc(-n2c(-c3cccnc3N)nc3ccc(-c4ccccc4)nc32)c(C)n1] |
| 150 | 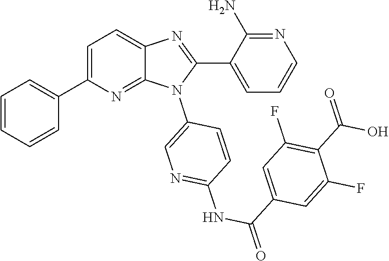 [SMILES: Nc1ncccc1-c1nc2ccc(-c3ccccc3)nc2n1-c1ccc(NC(=O)c2cc(F)c(C(=O)O)c(F)c2)nc1] |
| 151 | 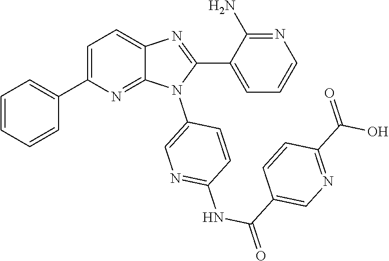 [SMILES: Nc1ncccc1-c1nc2ccc(-c3ccccc3)nc2n1-c1ccc(NC(=O)c2ccc(C(=O)O)nc2)nc1] |
| 152 | 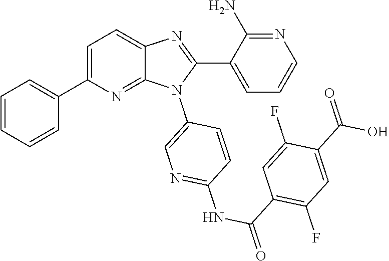 [SMILES: Nc1ncccc1-c1nc2ccc(-c3ccccc3)nc2n1-c1ccc(NC(=O)c2cc(F)c(C(=O)O)cc2F)nc1] |
| 153 | 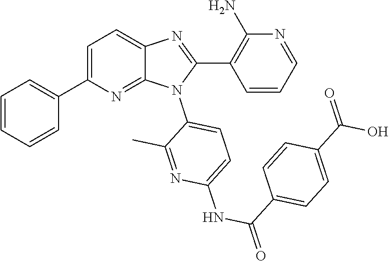 [SMILES: Cc1nc(NC(=O)c2ccc(C(=O)O)cc2)ccc1-n1c(-c2cccnc2N)nc2ccc(-c3ccccc3)nc21] |
| 154 | 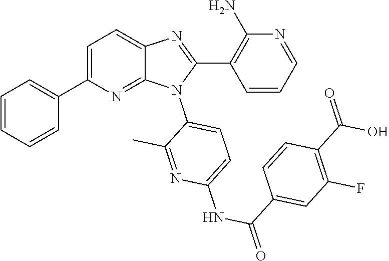 [SMILES: Cc1nc(NC(=O)c2ccc(C(=O)O)c(F)c2)ccc1-n1c(-c2cccnc2N)nc2ccc(-c3ccccc3)nc21] |
| 155 | 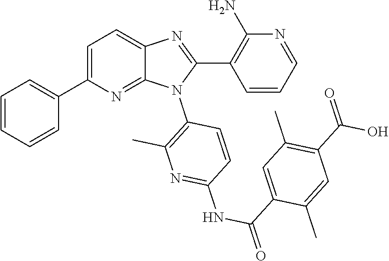 [SMILES: Cc1cc(C(=O)Nc2ccc(-n3c(-c4cccnc4N)nc4ccc(-c5ccccc5)nc43)c(C)n2)c(C)cc1C(=O)O] |
| 156 | 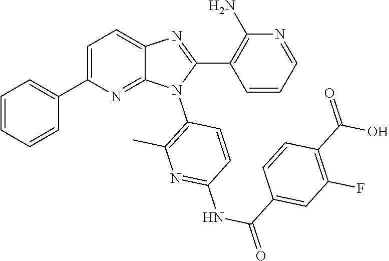 [SMILES: Cc1nc(NC(=O)c2ccc(C(=O)O)c(F)c2)ccc1-n1c(-c2cccnc2N)nc2ccc(-c3ccccc3)nc21] |
| 157 | 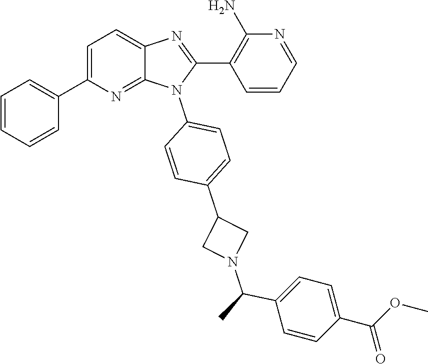 [SMILES: COC(=O)c1ccc([C@@H](C)N2CC(c3ccc(-n4c(-c5cccnc5N)nc5ccc(-c6ccccc6)nc54)cc3)C2)cc1] |
| 158 | 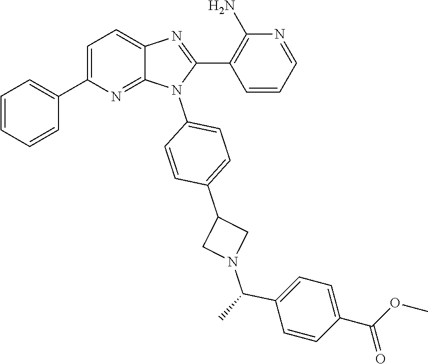 [SMILES: COC(=O)c1ccc([C@H](C)N2CC(c3ccc(-n4c(-c5cccnc5N)nc5ccc(-c6ccccc6)nc54)cc3)C2)cc1] |
| 159 | 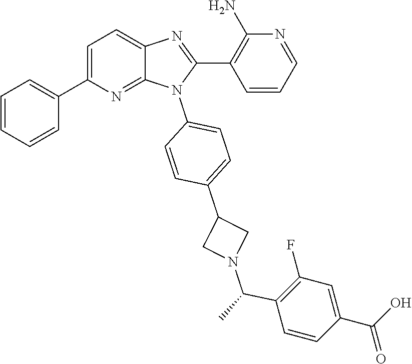 [SMILES: C[C@@H](c1ccc(C(=O)O)cc1F)N1CC(c2ccc(-n3c(-c4cccnc4N)nc4ccc(-c5ccccc5)nc43)cc2)C1] |
| 160 | 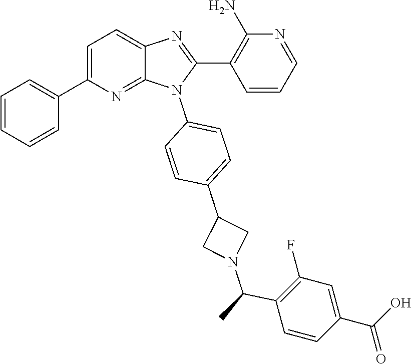 [SMILES: C[C@H](c1ccc(C(=O)O)cc1F)N1CC(c2ccc(-n3c(-c4cccnc4N)nc4ccc(-c5ccccc5)nc43)cc2)C1] |
| 161 | 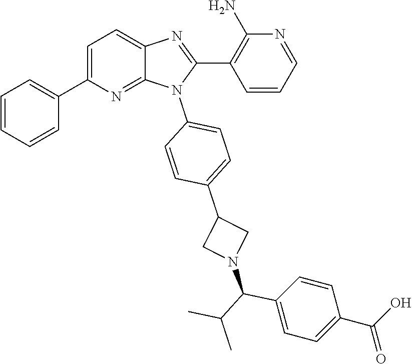 [SMILES: CC(C)[C@H](c1ccc(C(=O)O)cc1)N1CC(c2ccc(-n3c(-c4cccnc4N)nc4ccc(-c5ccccc5)nc43)cc2)C1] |
| 162 | 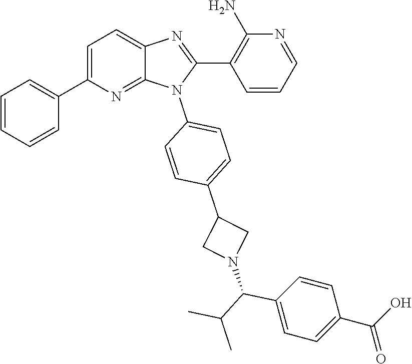 [SMILES: CC(C)[C@@H](c1ccc(C(=O)O)cc1)N1CC(c2ccc(-n3c(-c4cccnc4N)nc4ccc(-c5ccccc5)nc43)cc2)C1] |
| 163 | 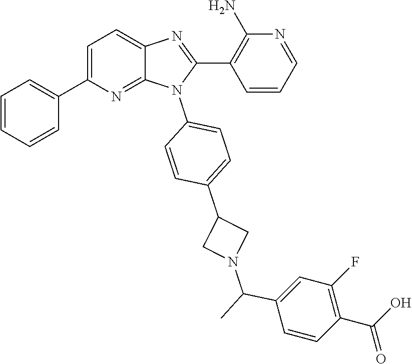 [SMILES: CC(c1ccc(C(=O)O)c(F)c1)N1CC(c2ccc(-n3c(-c4cccnc4N)nc4ccc(-c5ccccc5)nc43)cc2)C1] |
| 164 | 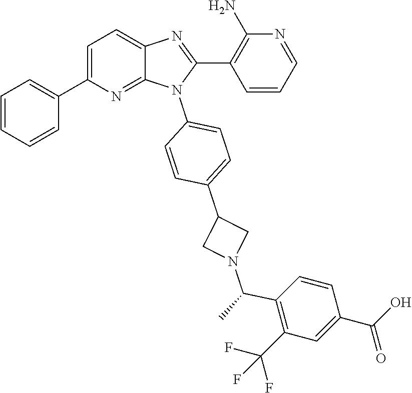 [SMILES: C[C@@H](c1ccc(C(=O)O)cc1C(F)(F)F)N1CC(c2ccc(-n3c(-c4cccnc4N)nc4ccc(-c5ccccc5)nc43)cc2)C1] |
| 165 | 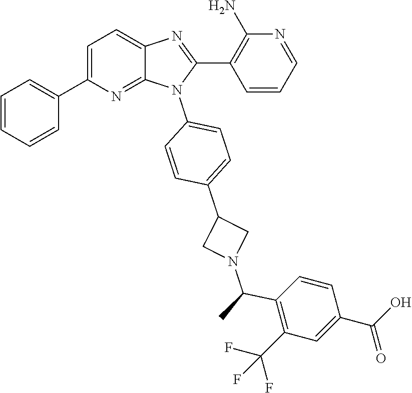 [SMILES: C[C@H](c1ccc(C(=O)O)cc1C(F)(F)F)N1CC(c2ccc(-n3c(-c4cccnc4N)nc4ccc(-c5ccccc5)nc43)cc2)C1] |
| 166 | 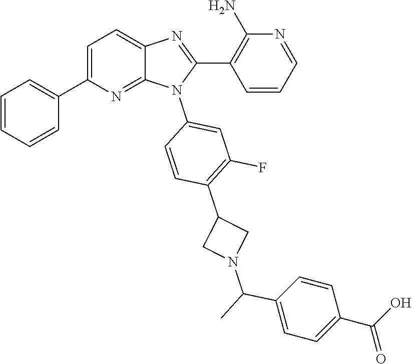 [SMILES: CC(c1ccc(C(=O)O)cc1)N1CC(c2ccc(-n3c(-c4cccnc4N)nc4ccc(-c5ccccc5)nc43)cc2F)C1] |
| 167 | 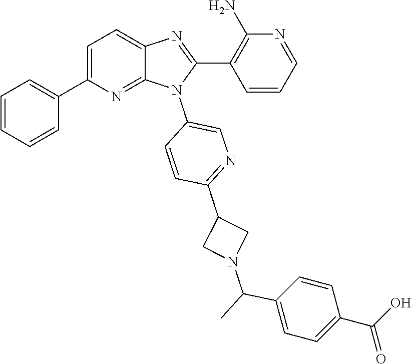 [SMILES: CC(c1ccc(C(=O)O)cc1)N1CC(c2ccc(-n3c(-c4cccnc4N)nc4ccc(-c5ccccc5)nc43)cn2)C1] |
| 168 | 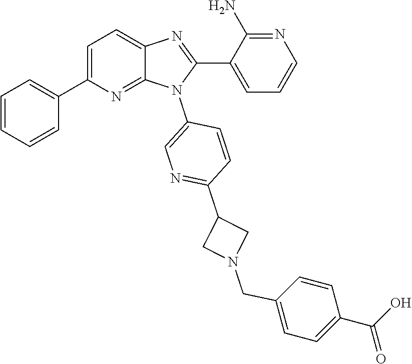 [SMILES: Nc1ncccc1-c1nc2ccc(-c3ccccc3)nc2n1-c1ccc(C2CN(Cc3ccc(C(=O)O)cc3)C2)nc1] |
| 169 | 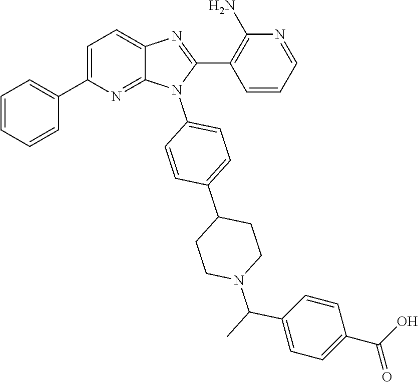 [SMILES: CC(c1ccc(C(=O)O)cc1)N1CCC(c2ccc(-n3c(-c4cccnc4N)nc4ccc(-c5ccccc5)nc43)cc2)CC1] |
| 170 | 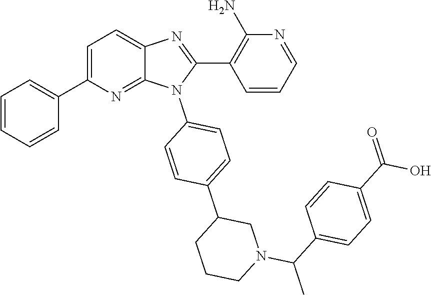 [SMILES: CC(c1ccc(C(=O)O)cc1)N1CCCC(c2ccc(-n3c(-c4cccnc4N)nc4ccc(-c5ccccc5)nc43)cc2)C1] |
| 171 | 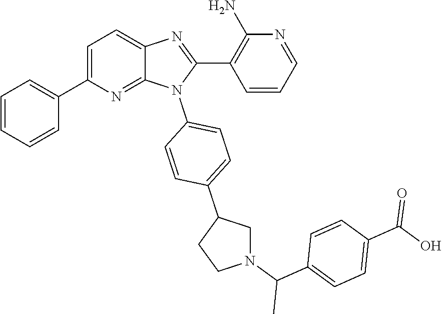 [SMILES: CC(c1ccc(C(=O)O)cc1)N1CCC(c2ccc(-n3c(-c4cccnc4N)nc4ccc(-c5ccccc5)nc43)cc2)C1] |
| 172 | 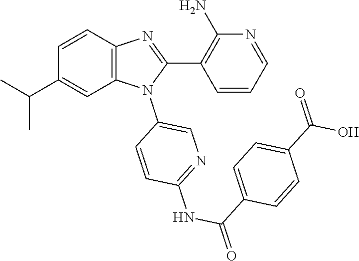 [SMILES: CC(C)c1ccc2nc(-c3cccnc3N)n(-c3ccc(NC(=O)c4ccc(C(=O)O)cc4)nc3)c2c1] |
| 173 | 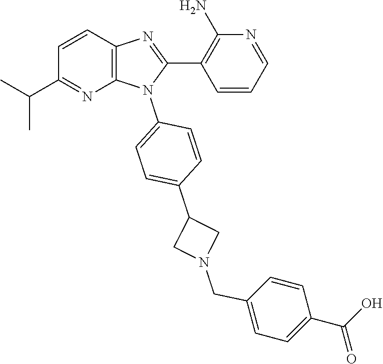 [SMILES: CC(C)c1ccc2nc(-c3cccnc3N)n(-c3ccc(C4CN(Cc5ccc(C(=O)O)cc5)C4)cc3)c2n1] |
| 174 | 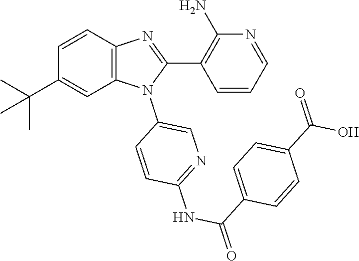 [SMILES: CC(C)(C)c1ccc2nc(-c3cccnc3N)n(-c3ccc(NC(=O)c4ccc(C(=O)O)cc4)nc3)c2c1] |
| 175 | 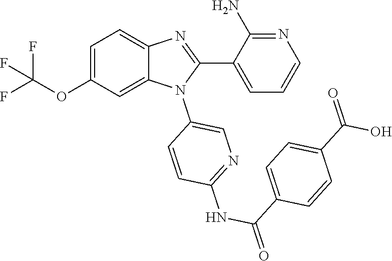 [SMILES: Nc1ncccc1-c1nc2ccc(OC(F)(F)F)cc2n1-c1ccc(NC(=O)c2ccc(C(=O)O)cc2)nc1] |
| 176 | 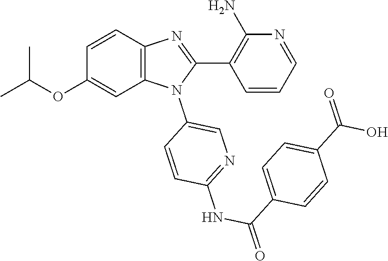 [SMILES: CC(C)Oc1ccc2nc(-c3cccnc3N)n(-c3ccc(NC(=O)c4ccc(C(=O)O)cc4)nc3)c2c1] |
| 177 | 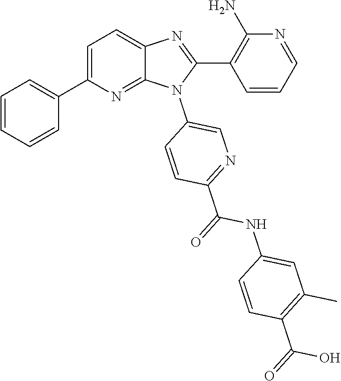 [SMILES: Cc1cc(NC(=O)c2ccc(-n3c(-c4cccnc4N)nc4ccc(-c5ccccc5)nc43)cn2)ccc1C(=O)O] |
| 178 | 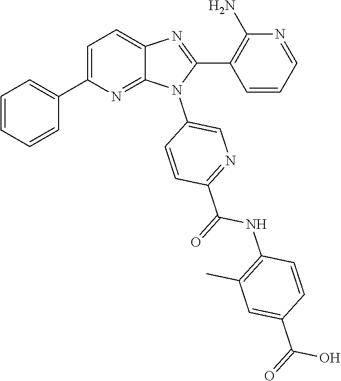 [SMILES: Cc1cc(C(=O)O)ccc1NC(=O)c1ccc(-n2c(-c3cccnc3N)nc3ccc(-c4ccccc4)nc32)cn1] |
| 179 | 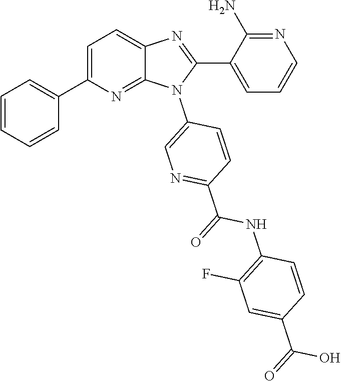 [SMILES: Nc1ncccc1-c1nc2ccc(-c3ccccc3)nc2n1-c1ccc(C(=O)Nc2ccc(C(=O)O)cc2F)nc1] |
| 180 | 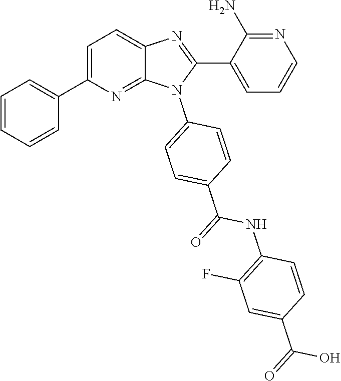 [SMILES: Nc1ncccc1-c1nc2ccc(-c3ccccc3)nc2n1-c1ccc(C(=O)Nc2ccc(C(=O)O)cc2F)cc1] |
| 181 | 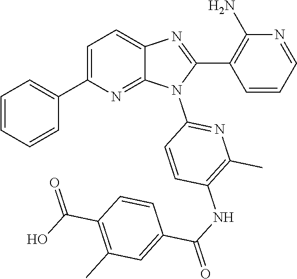 [SMILES: Cc1cc(C(=O)Nc2ccc(-n3c(-c4cccnc4N)nc4ccc(-c5ccccc5)nc43)nc2C)ccc1C(=O)O] |
| 182 | 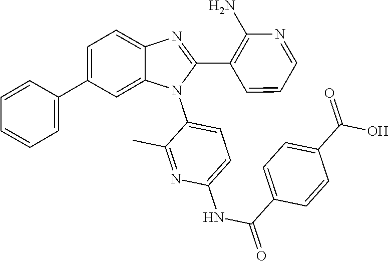 [SMILES: Cc1nc(NC(=O)c2ccc(C(=O)O)cc2)ccc1-n1c(-c2cccnc2N)nc2ccc(-c3ccccc3)cc21] |
| 183 | 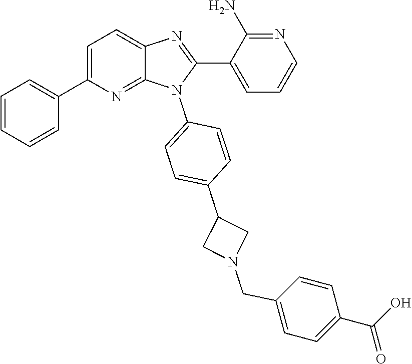 [SMILES: Nc1ncccc1-c1nc2ccc(-c3ccccc3)nc2n1-c1ccc(C2CN(Cc3ccc(C(=O)O)cc3)C2)cc1] |
| 184 | 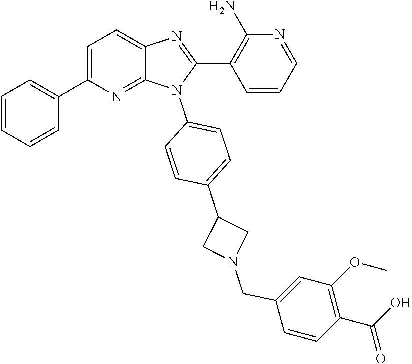 [SMILES: COc1cc(CN2CC(c3ccc(-n4c(-c5cccnc5N)nc5ccc(-c6ccccc6)nc54)cc3)C2)ccc1C(=O)O] |
| 185 | 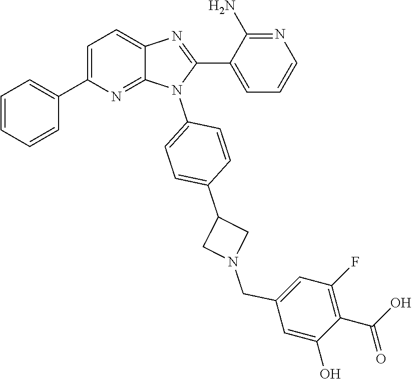 [SMILES: Nc1ncccc1-c1nc2ccc(-c3ccccc3)nc2n1-c1ccc(C2CN(Cc3cc(O)c(C(=O)O)c(F)c3)C2)cc1] |

### Synthetic Methods

Methods for preparing compounds described herein are illustrated in the following synthetic schemes (Scheme 1-Scheme 3). The schemes are given for the purpose of illustrating the invention, and not intended to limit the scope or spirit of the invention. Starting materials shown in the schemes can be obtained from commercial sources or can be prepared based on procedures described in the literature.

Scheme 1 illustrates a general method for preparing aminopyridinyl imidazo[4,5-b]pyridine compounds. Reaction of chloro-pyridine A with aniline B provides amine C. Reaction of amine C with aldehyde D provides imidazo[4,5-b]pyridine E, which is then converted to the final aminopyridinyl imidazo[4,5-b]pyridine compound F, after optional hydrolysis.

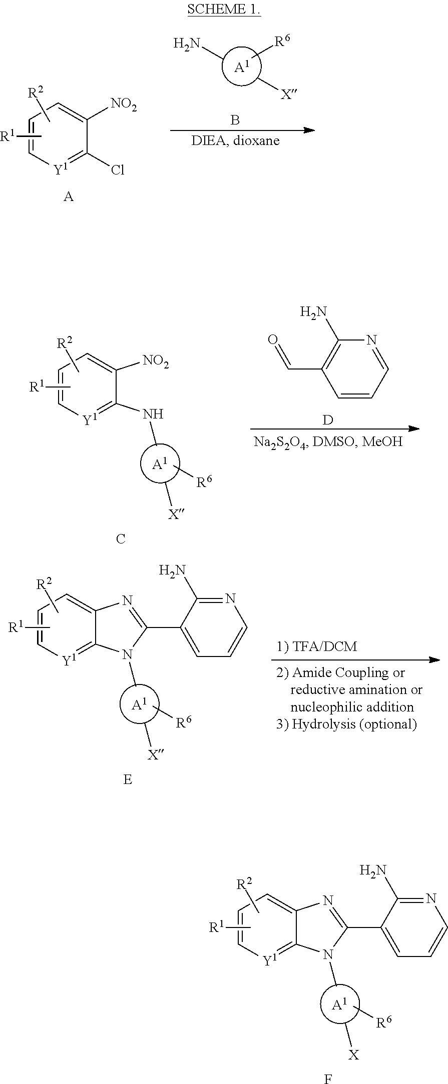

Scheme 2 illustrates a general method for preparing aminopyridinyl imidazo[4,5-b]pyridine compounds. Reaction of A with B provides C. Coupling of C with D provides E. Nitro E is reduced to provide amine F. Reaction of F with aldehyde G, after optional hydrolysis, provides the final imidazo[4,5-b]pyridine compound H.

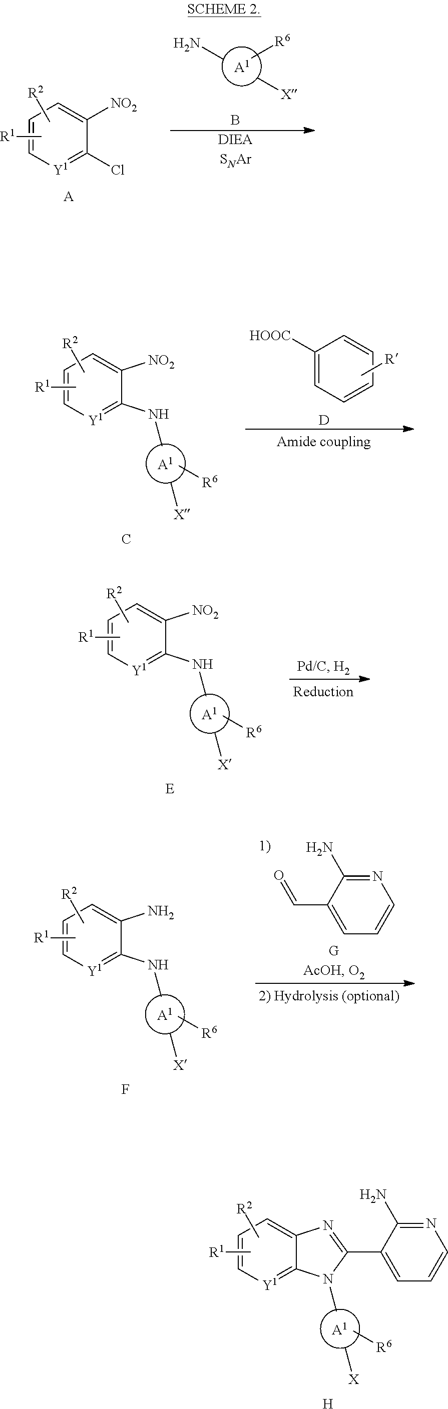

Scheme 3 illustrates a general method for preparing aminopyridinyl imidazo[4,5-b]pyridine compounds. Reaction of A with B provides C. Reaction of C with aldehyde D provides E. Suzuki coupling of boronate F to E provides G, which is then converted to provide the final imidazo[4,5-b]pyridine compound H, after optional hydrolysis.

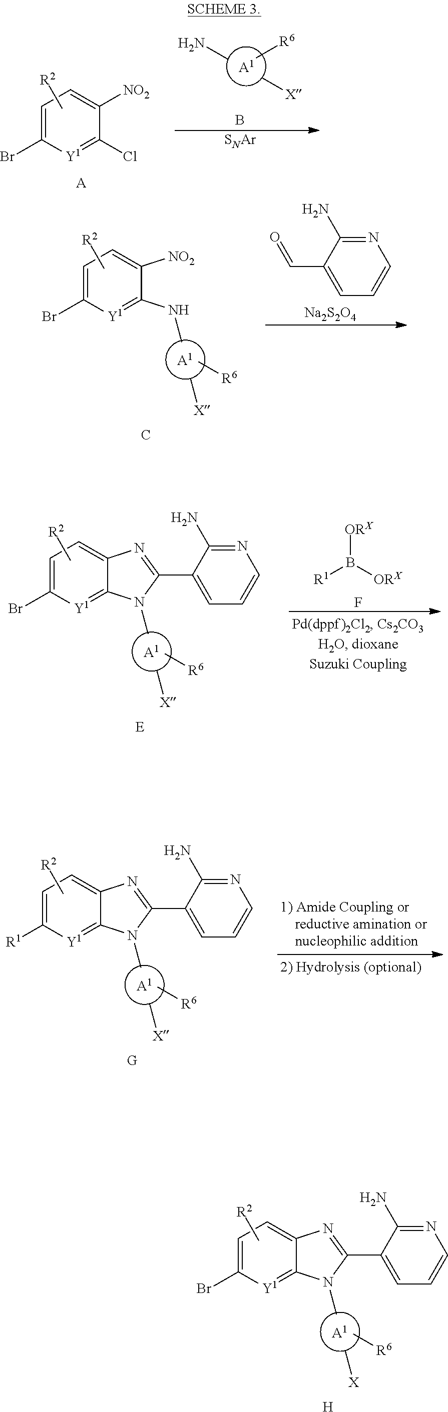

In the schemes (Scheme 1-Scheme 3) described above. R^{X} is a suitable substituent (e.g., H, alkyl, alkenyl, aryl) to form a boronic acid or ester, X is

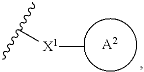

wherein “ [symbol] ” represents a bond to A¹, X′ and X″ refer to earlier stages in synthesis of X and Y¹, R¹, R², R⁶, A¹, X¹, and A² are as defined herein. In the schemes, it is understood by one skilled in the art of organic synthesis that the functionality present on various portions of the molecule should be compatible with the reagents and reactions proposed. Substituents not compatible with the reaction conditions will be apparent to one skilled in the art, and alternate methods are therefore indicated (for example, use of protecting groups or alternative reactions) Protecting group chemistry and strategy is well known in the art, for example, as described in detail in “Protecting Groups in Organic Synthesis”, T. W. Greene and P. G. M. Wuts, 3^{rd} edition, John Wiley & Sons, 1999, the entire contents of which are hereby incorporated by reference. The modular synthetic route illustrated in the schemes can also be readily modified by one of skill in the art to provide additional compounds by conducting functional group transformations on the intermediate and final compounds. Such functional group transformations are well known in the art, as described in, for example, “Comprehensive Organic Synthesis” (B. M. Trost & I. Fleming, eds., 1991-1992).

### II. Therapeutic Applications

Another aspect of the invention provides a method of treating a disease or disorder associated with aberrant AKT1 signaling, wherein the method comprises administering to a subject in need thereof a therapeutically effective amount of a compound described herein, such as a compound of Formula I, to treat the disease or disorder associated with aberrant AKT1 signaling. In certain embodiments, the particular compound of Formula I is a compound defined by one of the embodiments described in Section I, above. In certain embodiments, the disease or disorder associated with aberrant AKT1 signaling is an AKT1 E17K associated disease or disorder.

Another aspect of the invention provides a method of treating cancer, wherein the method comprises administering to a subject in need thereof a therapeutically effective amount of a compound described herein, such as a compound of Formula I, to treat the cancer. In certain embodiments, the particular compound of Formula I is a compound defined by one of the embodiments described in Section I, above.

Methods described herein may be further defined according to additional features, such as the identity of the cancer and/or the subject.

In certain embodiments, the cancer is ovarian cancer, uterine cancer, endometrial cancer, cervical cancer, prostate cancer, testicular cancer, breast cancer, brain cancer, lung cancer, oral cancer, esophageal cancer, head and neck cancer, stomach cancer, colon cancer, rectal cancer, skin cancer, sebaceous gland carcinoma, bile duct cancer, gallbladder cancer, liver cancer, pancreatic cancer, bladder cancer, urinary tract cancer, kidney cancer, eye cancer, thyroid cancer, lymphoma, leukemia, urothelial cancer, colorectal cancer, or glioblastoma multiforme.

In certain embodiments, the cancer is a solid tumor.

In certain embodiments, the cancer is a breast invasive carcinoma, colon adenocarcinoma, head and neck cancer, lung adenocarcinoma, rectal adenocarcinoma, acute myeloid leukemia, glioblastoma multiforme, brain lower grade glioma, colorectal cancer, uterine corpus endometrial carcinoma, cervical cancer, endocervical cancer, thyroid carcinoma, prostate adenocarcinoma, skin cutaneous melanoma, bladder urothelial carcinoma, head and neck squamous cell carcinoma, or stomach adenocarcinoma.

In certain embodiments, the cancer is an adenocarcinoma, squamous cell carcinoma, epithelial neoplasm, glioma, ductal neoplasm, lobular neoplasm, cystic neoplasm, mucinous neoplasm, or serous neoplasm, acinar cell neoplasm, basal cell neoplasm, fibroepithelial neoplasm, transitional cell papilloma, or transitional cell carcinoma.

In certain embodiments, the cancer is a cervical cancer, uterine cancer, breast cancer, thyroid cancer, prostate cancer, lung cancer, bladder cancer, skin cancer, stomach cancer, lymphoma, or leukemia.

In certain embodiments, the cancer is a lymphoma or leukemia.

In certain embodiments, the cancer has active PI3K signaling. In certain embodiments, the cancer has one or more mutations in phosphatase and tensin homolog (PTEN), phosphatidylinositol-4,5-bisphosphate 3-kinase catalytic subunit alpha (PIK3CA). phosphatidylinositol-4,5-bisphosphate 3-kinase catalytic subunit beta (PIK3CB), phosphatidylinositol-4,5-bisphosphate 3-kinase catalytic subunit delta (PIK3CD), mechanistic target of rapamycin kinase (mTOR), RPTOR independent companion of mTOR complex 2 (RICTOR), MAPK associated protein 1 (MAPKAPI), or 3-phosphoinositide dependent protein kinase 1 (PDPKI), or combinations thereof.

In certain embodiments, the cancer has an AKT1 mutation. In certain embodiments, the cancer has an AKT1 E17K mutation.

Exemplary cancers reported in the literature having an AKT1 E17K mutation include breast invasive carcinoma, uterine corpus endometrial carcinoma, colon adenocarcinoma, cervical and endocervical cancer, thyroid carcinoma, lung adenocarcinoma, prostate adenocarcinoma, skin cutaneous melanoma, bladder urothelial carcinoma, head and neck squamous cell carcinoma, and stomach adenocarcinoma.

Additionally, the AKT1 E17K mutation has been in reported in many sub-types of breast cancer including ductal, lobular, and in many combinations of HER2, estrogen receptor and progesterone receptor positivity. See, for example, BMC Cancer volume 16, Article number: 622 (2016)).

In certain embodiments, the cancer is a breast invasive carcinoma, colon adenocarcinoma, head and neck cancer, lung adenocarcinoma, rectal adenocarcinoma, acute myeloid leukemia, glioblastoma multiforme, brain lower grade glioma, colorectal cancer, or metastatic melanoma. In certain embodiments, the cancer is a melanoma.

In certain embodiments, the disorder is a cancer selected from the group consisting of ovarian cancer, uterine cancer, endometrial cancer, cervical cancer, prostate cancer, testicular cancer, breast cancer, brain cancer, lung cancer, oral cancer, esophageal cancer, head and neck cancer, stomach cancer, colon cancer, rectal cancer, skin cancer, sebaceous gland carcinoma, bile duct and gallbladder cancers, liver cancer, pancreatic cancer, bladder cancer, urinary tract cancer, kidney cancer, eye cancer, thyroid cancer, lymphoma, and leukemia.

In certain embodiments, the cancer is a solid tumor. In certain embodiments, the cancer is a sarcoma or carcinoma. In certain embodiments, the cancer is ovarian cancer, uterine cancer, endometrial cancer, cervical cancer, prostate cancer, testicular cancer, breast cancer, brain cancer, lung cancer, oral cancer, esophageal cancer, head and neck cancer, stomach cancer, colon cancer, rectal cancer, skin cancer, sebaceous gland carcinoma, bile duct and gallbladder cancers, liver cancer, pancreatic cancer, bladder cancer, urinary tract cancer, kidney cancer, eye cancer, thyroid cancer, lymphoma, or leukemia.

In certain embodiments, the cancer is prostate cancer, breast cancer, lung cancer, liver cancer, bladder cancer, urinary tract cancer, or eye cancer. In certain embodiments, the cancer is prostate cancer. In certain embodiments, the cancer is breast cancer. In certain embodiments, the cancer is lung cancer. In certain embodiments, the cancer is liver cancer. In certain embodiments, the cancer is bladder cancer. In certain embodiments, the cancer is urinary tract cancer. In certain embodiments, the cancer is eye cancer.

In certain embodiments, the cancer is squamous-cell carcinoma, basal cell carcinoma, adenocarcinoma, hepatocellular carcinomas, and renal cell carcinomas, cancer of the bladder, bowel, breast, cervix, colon, esophagus, head, kidney, liver, lung, neck, ovary, pancreas, prostate, and stomach; leukemias; benign and malignant lymphomas (e.g., Burkitt's lymphoma and Non-Hodgkin's lymphoma); benign and malignant melanomas; myeloproliferative diseases; sarcomas, including Ewing's sarcoma, hemangiosarcoma, Kaposi's sarcoma, liposarcoma, myosarcomas, peripheral neuroepithelioma, synovial sarcoma, gliomas, astrocytomas, oligodendrogliomas, ependymomas, gliobastomas, neuroblastomas, ganglioneuromas, gangliogliomas, medulloblastomas, pineal cell tumors, meningiomas, meningeal sarcomas, neurofibromas, and Schwannomas; bowel cancer, breast cancer, prostate cancer, cervical cancer, uterine cancer, lung cancer, ovarian cancer, testicular cancer, thyroid cancer, astrocytoma, esophageal cancer, pancreatic cancer, stomach cancer, liver cancer, colon cancer, melanoma; carcinosarcoma, Hodgkin's disease, Wilms' tumor and teratocarcinomas.

In certain embodiments, the cancer is a neuroblastoma, craniopharyngioma, glioma, glioblastoma, schwannoma, astrocytoma, oligodendroglioma, medulloblastoma, pinealoma, hemangioblastoma, retinoblastoma, ependymoma, chordoma, meningioma, medullary carcinoma, small cell lung carcinoma, papillary adenocarcinoma, papillary carcinoma, mesothelioma, nasopharyngeal carcinoma, acoustic neuroma, oral cancer, esophageal cancer, head and neck cancer, stomach cancer, colon cancer, rectal cancer, skin cancer, melanoma, sweat gland carcinoma, sebaceous gland carcinoma, squamous cell carcinoma, basal cell carcinoma, bile duct and gallbladder cancers, liver cancer, hepatocellular carcinoma, pancreatic cancer, bladder carcinoma, renal cell carcinoma, kidney cancer, Wilms' tumor, thyroid cancer, parathyroid tumor, synovioma, soft tissue sarcoma (e.g., rhabdomyosarcoma (RMS)), Kaposi sarcoma, synovial sarcoma, osteosarcoma, Ewing's sarcoma, malignant rhabdoid tumor, leiomyosarcoma, liposarcoma, lymphangioendothelio-sarcoma, lymphangiosarcoma, myxosarcoma, osteogenic sarcoma, fibrosarcoma, chondrosarcoma, or endotheliosarcoma.

In certain embodiments, the cancer is a lymphoma. In certain embodiments, the cancer is Burkitt's lymphoma, diffuse large B-cell lymphoma (DLBCL), follicular lymphoma, non-Hodgkin's lymphoma, lymphoid malignancies of T-cell or B-cell origin, peripheral T-cell lymphoma, adult T-cell leukemia-lymphoma, or Waldenström's macroglobulinemia.

In certain embodiments, the cancer is a leukemia. In certain embodiments, the cancer is acute leukemia, lymphoblastic leukemia, acute lymphoblastic leukemia, myelogenous leukemia, acute myelogenous leukemia, acute T-cell leukemia, chronic leukemia, chronic lymphocytic leukemia, chronic myelocytic leukemia, chronic myelogenous leukemia, polycythemia vera, multiple myeloma, or erythroleukemia.

In certain embodiments, the cancer is a myelodysplastic and/or myeloproliferative syndrome. In certain embodiments, the cancer is a myelodysplastic syndrome. In certain embodiments, the cancer is a myeloproliferative syndrome.

In certain embodiments, the cancer is a cancer or related myeloproliferative disorder selected from histiocytosis, essential thrombocythemia, myelofibrosis, heavy chain disease, and other malignancies and hyperproliferative disorders of the bladder, breast, colon, lung, ovaries, pancreas, prostate, skin and uterus.

In certain embodiments, the cancer is a B-cell non-Hodgkin's lymphoma, advanced solid tumor, soft tissue sarcoma, INII-deficient cancer, BAP1-deficient cancer, follicular lymphoma, relapsed/refractory follicular lymphoma, diffuse large B-cell lymphoma, relapsed/refractory diffuse large B-cell lymphoma, non-Hodgkin's lymphoma, pediatric non-Hodgkin's lymphoma, pediatric non-Hodgkin's lymphoma with EZH2, SMARCB1, or SMARCA4 mutation, histiocytic disorder, pediatric histiocytic disorder, pediatric histiocytic disorder with EZH2, SMARCB1, or SMARCA4 mutation, solid tumor with EZH2, SMARCB1, or SMARCA4 mutation, resistant prostate cancer, relapsed/refractory small-cell lung carcinoma, B-cell lymphoma, relapsed/refractory B-cell lymphoma, adult T-cell leukemia-lymphoma, or advanced diffuse large B-cell lymphoma.

In certain embodiments, the cancer is a malignant rhabdoid tumor, atypical teratoid rhabdoid tumor, epithelioid sarcoma, renal medullary carcinoma, pancreatic undifferentiated rhabdoid carcinoma, schwannoma, epithelioid malignant peripheral nerve sheath tumor, or diffuse intrinsic glioma.

In certain embodiments, the cancer is retinoblastoma multiforme, metastatic castration-resistant prostate cancer, prostate small cell neuroendocrine carcinoma, small-cell lung cancer, triple-negative breast cancer, hepatocellular carcinoma, bladder cancer, or urinary tract cancer.

In certain embodiments, the cancer is fibrosarcoma, myxosarcoma, liposarcoma, chondrosarcoma, osteogenic sarcoma, chordoma, angiosarcoma, endotheliosarcoma, lymphangiosarcoma, lymphangioendotheliosarcoma, Ewing's tumor, leiomyosarcoma, rhabdomyosarcoma, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, renal cell carcinoma, hepatoma, bile duct carcinoma, choriocarcinoma, seminoma, embryonal carcinoma, Wilms' tumor, epithelial carcinoma, glioma, astrocytoma, medulloblastoma, and hemangioblastoma. In certain embodiments, the cancer is a neuroblastoma, meningioma, hemangiopericytoma, multiple brain metastase, glioblastoma multiforms, glioblastoma, brain stem glioma, poor prognosis malignant brain tumor, malignant glioma, anaplastic astrocytoma, anaplastic oligodendroglioma, neuroendocrine tumor, rectal adeno carcinoma, Dukes C & D colorectal cancer, unresectable colorectal carcinoma, metastatic hepatocellular carcinoma, Kaposi's sarcoma, karotype acute myeloblastic leukemia, Hodgkin's lymphoma, non-Hodgkin's lymphoma, cutaneous T-Cell lymphoma, cutaneous B-Cell lymphoma, diffuse large B-Cell lymphoma, low grade follicular lymphoma, metastatic melanoma, localized melanoma, malignant mesothelioma, malignant pleural effusion mesothelioma syndrome, peritoneal carcinoma, papillary serous carcinoma, gynecologic sarcoma, soft tissue sarcoma, scelroderma, cutaneous vasculitis, Langerhans cell histiocytosis, leiomyosarcoma, fibrodysplasia ossificans progressive, hormone refractory prostate cancer, resected high-risk soft tissue sarcoma, unrescectable hepatocellular carcinoma. Waidenstrom's macroglobulinemia, smoldering myeloma, indolent myeloma, fallopian tube cancer, androgen independent prostate cancer, androgen dependent stage IV non-metastatic prostate cancer, hormone-insensitive prostate cancer, chemotherapy-insensitive prostate cancer, papillary thyroid carcinoma, follicular thyroid carcinoma, medullary thyroid carcinoma, or leiomyoma.

In certain embodiments, the cancer is a metastatic cancer. In certain embodiments, the cancer is a relapsed and/or refractory cancer.

In certain embodiments, the cancer is ovarian cancer, uterine cancer, gestational trophoblastic disease, endometrial cancer, cervical cancer, embryonal carcinoma, choriocarcinoma, prostate cancer (including hormone insensitive and castrate resistant prostate cancers), testicular tumors (including germ cell testicular cancer/seminoma), cystadenocarcinoma, breast cancer (including estrogen-receptor positive breast cancer), brain tumors (including neuroblastoma, craniopharyngioma, glioma, glioblastoma, schwannoma, astrocytoma, oligodendroglioma, medulloblastoma, and pinealoma), hemangioblastoma, retinoblastoma, ependymoma, chordoma, meningioma, medullary carcinoma, lung cancer (including small cell lung carcinoma, papillary adenocarcinomas, and papillary carcinoma), mesothelioma, nasopharyngeal carcinoma, acoustic neuroma, oral cancer, esophageal cancer, head and neck cancer, stomach cancer, colon cancer, rectal cancer, skin cancer, melanoma, sweat gland carcinoma, sebaceous gland carcinoma, squamous cell carcinoma, basal cell carcinoma, bile duct and gallbladder cancers, liver cancer, hepatocellular carcinoma, pancreatic cancer, bladder carcinoma, renal cell carcinoma, kidney cancer, Wilms' tumor, thyroid cancer, parathyroid tumor, synovioma, soft tissue sarcoma (e.g., rhabdomyosarcoma (RMS)), Kaposi sarcoma, synovial sarcoma, osteosarcoma, Ewing's sarcoma, malignant rhabdoid tumor, leiomyosarcoma, liposarcoma, lymphangioendothelio-sarcoma, lymphangiosarcoma, myxosarcoma, osteogenic sarcoma, fibrosarcoma, chondrosarcoma, or endotheliosarcoma.

Methods of Treating Diseases or Disorders Associated with Active PI3K Signaling

A method of treating a disease or disorder associated with active PI3K signaling. The method comprises administering to a subject in need thereof a therapeutically effective amount of a compound described herein, such as a compound of Formula I, to treat the disease or disorder. In certain embodiments, the disease or disorder features hyperactive PI3K signaling.

### Subjects

In certain embodiments, the subject is a human. In certain embodiments, the subject is an adult human. In certain embodiments, the subject is a pediatric human.

### Methods of Inhibiting AKT1 Activity

Another aspect of the invention provides a method of inhibiting AKT1 activity. The method comprises contacting an AKT1 with an effective amount of a compound described herein, such as a compound of Formula I, to thereby inhibit the AKT1 activity.

In certain embodiments, the AKT1 is AKT1 E17K.

In certain embodiments, the AKT1 protein is AKT1 E17K.

### Medical Uses

Another aspect of the invention provides for the use of a compound described herein (such as a compound of Formula I, or other compounds in Section I) in the manufacture of a medicament. In certain embodiments, the medicament is for treating a disorder described herein, such as cancer.

Another aspect of the invention provides for the use of a compound described herein (such as a compound of Formula I, or other compounds in Section I) for treating a medical disorder, such as a medical disorder described herein, such as cancer.

### IV. Combination Therapy

Another aspect of the invention provides for combination therapy. Compounds described herein (such as a compound of Formula I, or other compounds in Section I) or their pharmaceutically acceptable salts may be used in combination with additional therapeutic agents to treat medical disorders, such as an autoimmune disorder or a cancer.

In certain embodiments, the present invention provides a method of treating a disclosed disease or condition comprising administering to a patient in need thereof an effective amount of a compound disclosed herein or a pharmaceutically acceptable salt thereof and co-administering simultaneously or sequentially an effective amount of one or more additional therapeutic agents, such as those described herein. In certain embodiments, the method includes co-administering one additional therapeutic agent. In certain embodiments, the method includes co-administering two additional therapeutic agents. In certain embodiments, the combination of the disclosed compound and the additional therapeutic agent or agents acts synergistically.

One or more other therapeutic agent may be administered separately from a compound or composition of the invention, as part of a multiple dosage regimen. Alternatively, one or more other therapeutic agents may be part of a single dosage form, mixed together with a compound of this invention in a single composition. If administered as a multiple dosage regime, one or more other therapeutic agent and a compound or composition of the invention may be administered simultaneously, sequentially or within a period of time from one another, for example within 1, 2, 3, 4, 5, 6, 7, 8, 9, 10, 11, 12, 13, 14, 15, 16, 17, 18, 18, 20, 21, 22, 23, or 24 hours from one another. In certain embodiments, one or more other therapeutic agent and a compound or composition of the invention are administered as a multiple dosage regimen more than 24 hours apart.

### Anti-Cancer Agents

Exemplary therapeutic agents that may be used as part of a combination therapy in treating cancer, include, for example, mitomycin, tretinoin, ribomustin, gemcitabine, vincristine, etoposide, cladribine, mitobronitol, methotrexate, doxorubicin, carboquone, pentostatin, nitracrine, zinostatin, cetrorelix, letrozole, raltitrexed, daunorubicin, fadrozole, fotemustine, thymalfasin, sobuzoxane, nedaplatin, cytarabine, bicalutamide, vinorelbine, vesnarinone, aminoglutethimide, amsacrine, proglumide, elliptinium acetate, ketanserin, doxifluridine, etretinate, isotretinoin, streptozocin, nimustine, vindesine, flutamide, drogenil, butocin, carmofur, razoxane, sizofilan, carboplatin, mitolactol, tegafur, ifosfamide, prednimustine, picibanil, levamisole, teniposide, improsulfan, enocitabine, lisuride, oxymetholone, tamoxifen, progesterone, mepitiostane, epitiostanol, formestane, interferon-alpha, interferon-2 alpha, interferon-beta, interferon-gamma, colony stimulating factor-1, colony stimulating factor-2, denileukin diftitox, interleukin-2, and leutinizing hormone releasing factor.

Radiation therapy may also be used as part of a combination therapy.

An additional class of agents that may be used as part of a combination therapy in treating cancer is immune checkpoint inhibitors (also referred to as immune checkpoint blockers). Immune checkpoint inhibitors are a class of therapeutic agents that have the effect of blocking immune checkpoints. See, for example, Pardoll in Nature Reviews Cancer (2012) vol. 12, pages 252-264. Exemplary immune checkpoint inhibitors include agents that inhibit one or more of (i) cytotoxic T-lymphocyte-associated antigen 4 (CTLA4), (ii) programmed cell death protein 1 (PD1), (iii) PDLI, (iv) LAB3, (v) B7-H3, (vi) B7-H4, and (vii) TIM3. The CTLA4 inhibitor ipilumumab has been approved by the United States Food and Drug Administration for treating melanoma. In certain embodiments, the immune checkpoint inhibitor comprises pembrolizumab.

Yet other agents that may be used as part of a combination therapy in treating cancer are monoclonal antibody agents that target non-checkpoint targets (e.g., herceptin) and non-cytotoxic agents (e.g., tyrosine-kinase inhibitors).

Accordingly, another aspect of the invention provides a method of treating cancer in a patient, where the method comprises administering to the patient in need thereof (i) a therapeutically effective amount of a compound described herein and (ii) a second anti-cancer agent, in order to treat the cancer, where the second therapeutic agent may be one of the additional therapeutic agents described above (e.g., mitomycin, tretinoin, ribomustin, gemcitabine, an immune checkpoint inhibitor, or a monoclonal antibody agent that targets non-checkpoint targets) or one of the following:
an inhibitor selected from an ALK Inhibitor, an ATR Inhibitor, an A2A Antagonist, a Base Excision Repair Inhibitor, a Ber-Abl Tyrosine Kinase Inhibitor, a Bruton's Tyrosine Kinase Inhibitor, a CDC7 Inhibitor, a CHKI Inhibitor, a Cyclin-Dependent Kinase Inhibitor, a DNA-PK Inhibitor, an Inhibitor of both DNA-PK and mTOR, a DNMT1 Inhibitor, a DNMT1 Inhibitor plus 2-chloro-deoxyadenosine, an HDAC Inhibitor, a Hedgehog Signaling Pathway Inhibitor, an IDO Inhibitor, a JAK Inhibitor, a mTOR Inhibitor, a MEK Inhibitor, a MELK Inhibitor, a MTH1 Inhibitor, a PARP Inhibitor, a Phosphoinositide 3-Kinase Inhibitor, an Inhibitor of both PARPI and DHODH, a Proteasome Inhibitor, a Topoisomerase-II Inhibitor, a Tyrosine Kinase Inhibitor, a VEGFR Inhibitor, and a WEE1 Inhibitor;
an agonist of OX40, CD137, CD40, GITR, CD27, HVEM, TNFRSF25, or ICOS;
a therapeutic antibody targeting one of the following: CD20, CD30, CD33, CD52, EpCAM, CEA, gpA33, a mucin, TAG-72, CAIX, PSMA, a folate-binding protein, a ganglioside, Le, VEGF, VEGFR, VEGFR2, integrin αVβ3, integrin α5β1, EGFR, ERBB2, ERBB3, MET, IGFIR, EPHA3, TRAILR1, TRAILR2, RANKL, FAP, tenascin, CD19, KIR, NKG2A, CD47, CEACAMI, c-MET, VISTA, CD73, CD38, BAFF, interleukin-1 beta, B4GALNT1, interleukin-6, and interleukin-6 receptor:
a cytokine selected from IL-12, IL-15, GM-CSF, and G-CSF;
a therapeutic agent selected from sipuleucel-T, aldesleukin (a human recombinant interleukin-2 product having the chemical name des-alanyl-1, serine-125 human interleukin-2), dabrafenib (a kinase inhibitor having the chemical name N-{3-[5-(2-aminopyrimidin-4-yl)-2-tert-butyl-1,3-thiazol-4-yl]-2-fluorophenyl}-2,6-difluorobenzenesulfonamide), vemurafenib (a kinase inhibitor having the chemical name propane-1-sulfonic acid {3-[5-(4-chlorophenyl)-1H-pyrrolo[2,3-6]pyridine-3-carbonyl]-2,4-difluoro-phenyl}-amide), and 2-chloro-deoxyadenosine; or
a placental growth factor, an antibody-drug conjugate, an oncolytic virus, or an anti-cancer vaccine.

In certain embodiments, the second anti-cancer agent is an ALK Inhibitor. In certain embodiments, the second anti-cancer agent is an ALK Inhibitor comprising ceritinib or crizotinib. In certain embodiments, the second anti-cancer agent is an ATR Inhibitor. In certain embodiments, the second anti-cancer agent is an ATR Inhibitor comprising AZD6738 or VX-970. In certain embodiments, the second anti-cancer agent is an A2A Antagonist. In certain embodiments, the second anti-cancer agent is a Base Excision Repair Inhibitor comprising methoxyamine. In certain embodiments, the second anti-cancer agent is a Base Excision Repair Inhibitor, such as methoxyamine. In certain embodiments, the second anti-cancer agent is a Bcr-Abl Tyrosine Kinase Inhibitor. In certain embodiments, the second anti-cancer agent is a Bcr-Abl Tyrosine Kinase Inhibitor comprising dasatinib or nilotinib. In certain embodiments, the second anti-cancer agent is a Bruton's Tyrosine Kinase Inhibitor. In certain embodiments, the second anti-cancer agent is a Bruton's Tyrosine Kinase Inhibitor comprising ibrutinib. In certain embodiments, the second anti-cancer agent is a CDC7 Inhibitor. In certain embodiments, the second anti-cancer agent is a CDC7 Inhibitor comprising RXDX-103 or AS-141.

In certain embodiments, the second anti-cancer agent is a CHKI Inhibitor. In certain embodiments, the second anti-cancer agent is a CHKI Inhibitor comprising MK-8776, ARRY-575, or SAR-020106. In certain embodiments, the second anti-cancer agent is a Cyclin-Dependent Kinase Inhibitor. In certain embodiments, the second anti-cancer agent is a Cyclin-Dependent Kinase Inhibitor comprising palbociclib. In certain embodiments, the second anti-cancer agent is a DNA-PK Inhibitor. In certain embodiments, the second anti-cancer agent is a DNA-PK Inhibitor comprising MSC2490484A. In certain embodiments, the second anti-cancer agent is Inhibitor of both DNA-PK and mTOR. In certain embodiments, the second anti-cancer agent comprises CC-115.

In certain embodiments, the second anti-cancer agent is a DNMT1 Inhibitor. In certain embodiments, the second anti-cancer agent is a DNMT1 Inhibitor comprising decitabine, RX-3117, guadecitabine, NUC-8000, or azacytidine. In certain embodiments, the second anti-cancer agent comprises a DNMT1 Inhibitor and 2-chloro-deoxyadenosine. In certain embodiments, the second anti-cancer agent comprises ASTX-727.

In certain embodiments, the second anti-cancer agent is a HDAC Inhibitor. In certain embodiments, the second anti-cancer agent is a HDAC Inhibitor comprising OBP-801, CHR-3996, etinostate, resminostate, pracinostat, CG-200745, panobinostat, romidepsin, mocetinostat, belinostat, AR-42, ricolinostat, KA-3000, or ACY-241.

In certain embodiments, the second anti-cancer agent is a Hedgehog Signaling Pathway Inhibitor. In certain embodiments, the second anti-cancer agent is a Hedgehog Signaling Pathway Inhibitor comprising sonidegib or vismodegib. In certain embodiments, the second anti-cancer agent is an IDO Inhibitor. In certain embodiments, the second anti-cancer agent is an IDO Inhibitor comprising INCB024360. In certain embodiments, the second anti-cancer agent is a JAK Inhibitor. In certain embodiments, the second anti-cancer agent is a JAK Inhibitor comprising ruxolitinib or tofacitinib. In certain embodiments, the second anti-cancer agent is a mTOR Inhibitor. In certain embodiments, the second anti-cancer agent is a mTOR Inhibitor comprising everolimus or temsirolimus. In certain embodiments, the second anti-cancer agent is a MEK Inhibitor. In certain embodiments, the second anti-cancer agent is a MEK Inhibitor comprising cobimetinib or trametinib. In certain embodiments, the second anti-cancer agent is a MELK Inhibitor. In certain embodiments, the second anti-cancer agent is a MELK Inhibitor comprising ARN-7016, APTO-500, or OTS-167. In certain embodiments, the second anti-cancer agent is a MTH1 Inhibitor. In certain embodiments, the second anti-cancer agent is a MTH1 Inhibitor comprising(S)-crizotinib, TH287, or TH588.

In certain embodiments, the second anti-cancer agent is a PARP Inhibitor. In certain embodiments, the second anti-cancer agent is a PARP Inhibitor comprising MP-124, olaparib, BGB-290, talazoparib, veliparib, niraparib, E7449, rucaparb, or ABT-767. In certain embodiments, the second anti-cancer agent is a Phosphoinositide 3-Kinase Inhibitor. In certain embodiments, the second anti-cancer agent is a Phosphoinositide 3-Kinase Inhibitor comprising idelalisib. In certain embodiments, the second anti-cancer agent is an inhibitor of both PARPI and DHODH (i.e., an agent that inhibits both poly ADP ribose polymerase 1 and dihydroorotate dehydrogenase).

In certain embodiments, the second anti-cancer agent is a Proteasome Inhibitor. In certain embodiments, the second anti-cancer agent is a Proteasome Inhibitor comprising bortezomib or carfilzomib. In certain embodiments, the second anti-cancer agent is a Topoisomerase-II Inhibitor. In certain embodiments, the second anti-cancer agent is a Topoisomerase-II Inhibitor comprising vosaroxin.

In certain embodiments, the second anti-cancer agent is a Tyrosine Kinase Inhibitor. In certain embodiments, the second anti-cancer agent is a Tyrosine Kinase Inhibitor comprising bosutinib, cabozantinib, imatinib or ponatinib. In certain embodiments, the second anti-cancer agent is a VEGFR Inhibitor. In certain embodiments, the second anti-cancer agent is a VEGFR Inhibitor comprising regorafenib. In certain embodiments, the second anti-cancer agent is a WEE1 Inhibitor. In certain embodiments, the second anti-cancer agent is a WEE1 Inhibitor comprising AZD1775.

In certain embodiments, the second anti-cancer agent is an agonist of OX40. CD137, CD40, GITR, CD27, HVEM, INFRSF25, or ICOS. In certain embodiments, the second anti-cancer agent is a therapeutic antibody selected from the group consisting of rituximab, ibritumomab tiuxetan, tositumomab, obinutuzumab, ofatumumab, brentuximab vedotin, gemtuzumab ozogamicin, alemtuzumab, IGN101, adecatumumab, labetuzumab, huA33, pemtumomab, oregovomab, minetumomab, cG250, J591, Mov18, farletuzumab, 3F8, ch14.18, KW-2871, hu3S193, IgN311, bevacizumab, IM-2C₆, pazopanib, sorafenib, axitinib, CDP791, lenvatinib, ramucirumab, etaracizumab, volociximab, cetuximab, panitumumab, nimotuzumab, 806, afatinib, erlotinib, gefitinib, osimertinib, vandetanib, trastuzumab, pertuzumab, MM-121, AMG 102, METMAB, SCH 900105, AVE1642, IMC-A12, MK-0646, R1507, CP 751871, KB004, IIIA-4, mapatumumab, HGS-ETR2, CS-1008, denosumab, sibrotuzumab, F19, 81C6, MEDI551, lirilumab, MED19447, daratumumab, belimumab, canakinumab, dinutuximab, siltuximab, and tocilizumab.

In certain embodiments, the second anti-cancer agent is a placental growth factor. In certain embodiments, the second anti-cancer agent is a placental growth factor comprising ziv-aflibercept. In certain embodiments, the second anti-cancer agent is an antibody-drug conjugate. In certain embodiments, the second anti-cancer agent is an antibody-drug conjugate selected from the group consisting of brentoxumab vedotin and trastuzumab emtransine.

In certain embodiments, the second anti-cancer agent is an oncolytic virus. In certain embodiments, the second anti-cancer agent is the oncolytic virus talimogene laherparepvec. In certain embodiments, the second anti-cancer agent is an anti-cancer vaccine. In certain embodiments, the second anti-cancer agent is an anti-cancer vaccine selected from the group consisting of a GM-CSF tumor vaccine, a STING/GM-CSF tumor vaccine, and NY-ESO-1. In certain embodiments, the second anti-cancer agent is a cytokine selected from IL-12, IL-15, GM-CSF, and G-CSF.

In certain embodiments, the second anti-cancer agent is a therapeutic agent selected from sipuleucel-T, aldesleukin (a human recombinant interleukin-2 product having the chemical name des-alanyl-1, serine-125 human interleukin-2), dabrafenib (a kinase inhibitor having the chemical name N-{3-[5-(2-aminopyrimidin-4-yl)-2-tert-butyl-1,3-thiazol-4-yl]-2-fluorophenyl}-2,6-difluorobenzenesulfonamide), vemurafenib (a kinase inhibitor having the chemical name propane-1-sulfonic acid {3-[S-(4-chlorophenyl)-1H-pyrrolo[2,3-b]pyridine-3-carbonyl]-2,4-difluoro-phenyl}-amide), and 2-chloro-deoxyadenosine.

In certain embodiments, the second anti-cancer agent is a hormone therapy agent. Exemplary hormone therapy agents include, for example, fulvestrant (faslodex) and other agents that target estrogen receptor and estrogen signaling. In certain embodiments, the second therapeutic agent is a CDK4/6 inhibitor.

### Additional Considerations

The doses and dosage regimen of the active ingredients used in the combination therapy may be determined by an attending clinician. In certain embodiments, the compound described herein (such as a compound of Formula I, or other compounds in Section I) and the additional therapeutic agent(s) are administered in doses commonly employed when such agents are used as monotherapy for treating the disorder. In other embodiments, the compound described herein (such as a compound of Formula I, or other compounds in Section I) and the additional therapeutic agent(s) are administered in doses lower than the doses commonly employed when such agents are used as monotherapy for treating the disorder. In certain embodiments, the compound described herein (such as a compound of Formula I, or other compounds in Section I) and the additional therapeutic agent(s) are present in the same composition, which is suitable for oral administration.

In certain embodiments, the compound described herein (such as a compound of Formula I, or other compounds in Section I) and the additional therapeutic agent(s) may act additively or synergistically. A synergistic combination may allow the use of lower dosages of one or more agents and/or less frequent administration of one or more agents of a combination therapy. A lower dosage or less frequent administration of one or more agents may lower toxicity of the therapy without reducing the efficacy of the therapy.

Another aspect of this invention is a kit comprising a therapeutically effective amount of the compound described herein (such as a compound of Formula I, or other compounds in Section I), a pharmaceutically acceptable carrier, vehicle or diluent, and optionally at least one additional therapeutic agent listed above.

### III. Pharmaceutical Compositions and Dosing Considerations

As indicated above, the invention provides pharmaceutical compositions, which comprise a therapeutically-effective amount of one or more of the compounds described above, formulated together with one or more pharmaceutically acceptable carriers (additives) and/or diluents. The pharmaceutical compositions may be specially formulated for administration in solid or liquid form, including those adapted for the following: (1) oral administration, for example, drenches (aqueous or non-aqueous solutions or suspensions), tablets, e.g., those targeted for buccal, sublingual, and systemic absorption, boluses, powders, granules, pastes for application to the tongue; (2) parenteral administration, for example, by subcutaneous, intramuscular, intravenous or epidural injection as, for example, a sterile solution or suspension, or sustained-release formulation; (3) topical application, for example, as a cream, ointment, or a controlled-release patch or spray applied to the skin, (4) intravaginally or intrarectally, for example, as a pessary, cream or foam; (5) sublingually; (6) ocularly; (7) transdermally; or (8) nasally. In certain embodiments, the invention provides a pharmaceutical composition comprising a compound described herein (such as a compound of Formula I, or other compounds in Section I) and a pharmaceutically acceptable carrier.

The phrase “therapeutically effective amount” as used herein means that amount of a compound, material, or composition comprising a compound of the present invention which is effective for producing some desired therapeutic effect in at least a sub-population of cells in an animal at a reasonable benefit/risk ratio applicable to any medical treatment.

The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

Wetting agents, emulsifiers and lubricants, such as sodium lauryl sulfate and magnesium stearate, as well as coloring agents, release agents, coating agents, sweetening, flavoring and perfuming agents, preservatives and antioxidants can also be present in the compositions.

Examples of pharmaceutically-acceptable antioxidants include: (1) water soluble antioxidants, such as ascorbic acid, cysteine hydrochloride, sodium bisulfate, sodium metabisulfite, sodium sulfite and the like; (2) oil-soluble antioxidants, such as ascorbyl palmitate, butylated hydroxyanisole (BHA), butylated hydroxytoluene (BHT), lecithin, propyl gallate, alpha-tocopherol, and the like; and (3) metal chelating agents, such as citric acid, ethylenediamine tetraacetic acid (EDTA), sorbitol, tartaric acid, phosphoric acid, and the like.

Formulations of the present invention include those suitable for oral, nasal, topical (including buccal and sublingual), rectal, vaginal and/or parenteral administration. The formulations may conveniently be presented in unit dosage form and may be prepared by any methods well known in the art of pharmacy. The amount of active ingredient which can be combined with a carrier material to produce a single dosage form will vary depending upon the host being treated, the particular mode of administration. The amount of active ingredient which can be combined with a carrier material to produce a single dosage form will generally be that amount of the compound which produces a therapeutic effect. Generally, out of one hundred percent, this amount will range from about 0.1 percent to about ninety-nine percent of active ingredient, preferably from about 5 percent to about 70 percent, most preferably from about 10 percent to about 30 percent.

In certain embodiments, a formulation of the present invention comprises an excipient selected from the group consisting of cyclodextrins, celluloses, liposomes, micelle forming agents, e.g., bile acids, and polymeric carriers, e.g., polyesters and polyanhydrides; and a compound of the present invention. In certain embodiments, an aforementioned formulation renders orally bioavailable a compound of the present invention.

Methods of preparing these formulations or compositions include the step of bringing into association a compound of the present invention with the carrier and, optionally, one or more accessory ingredients. In general, the formulations are prepared by uniformly and intimately bringing into association a compound of the present invention with liquid carriers, or finely divided solid carriers, or both, and then, if necessary, shaping the product.

Formulations of the invention suitable for oral administration may be in the form of capsules, cachets, pills, tablets, lozenges (using a flavored basis, usually sucrose and acacia or tragacanth), powders, granules, or as a solution or a suspension in an aqueous or non-aqueous liquid, or as an oil-in-water or water-in-oil liquid emulsion, or as an elixir or syrup, or as pastilles (using an inert base, such as gelatin and glycerin, or sucrose and acacia) and/or as mouth washes and the like, each containing a predetermined amount of a compound of the present invention as an active ingredient. A compound of the present invention may also be administered as a bolus, electuary or paste.

In solid dosage forms of the invention for oral administration (capsules, tablets, pills, dragees, powders, granules, trouches and the like), the active ingredient is mixed with one or more pharmaceutically-acceptable carriers, such as sodium citrate or dicalcium phosphate, and/or any of the following: (1) fillers or extenders, such as starches, lactose, sucrose, glucose, mannitol, and/or silicic acid; (2) binders, such as, for example, carboxymethylcellulose, alginates, gelatin, polyvinyl pyrrolidone, sucrose and/or acacia; (3) humectants, such as glycerol; (4) disintegrating agents, such as agar-agar, calcium carbonate, potato or tapioca starch, alginic acid, certain silicates, and sodium carbonate; (5) solution retarding agents, such as paraffin; (6) absorption accelerators, such as quaternary ammonium compounds and surfactants, such as poloxamer and sodium lauryl sulfate; (7) wetting agents, such as, for example, cetyl alcohol, glycerol monostearate, and non-ionic surfactants; (8) absorbents, such as kaolin and bentonite clay; (9) lubricants, such as talc, calcium stearate, magnesium stearate, solid polyethylene glycols, sodium lauryl sulfate, zinc stearate, sodium stearate, stearic acid, and mixtures thereof; (10) coloring agents; and (11) controlled release agents such as crospovidone or ethyl cellulose. In the case of capsules, tablets and pills, the pharmaceutical compositions may also comprise buffering agents. Solid compositions of a similar type may also be employed as fillers in soft and hard-shelled gelatin capsules using such excipients as lactose or milk sugars, as well as high molecular weight polyethylene glycols and the like.

A tablet may be made by compression or molding, optionally with one or more accessory ingredients. Compressed tablets may be prepared using binder (for example, gelatin or hydroxypropylmethyl cellulose), lubricant, inert diluent, preservative, disintegrant (for example, sodium starch glycolate or cross-linked sodium carboxymethyl cellulose), surface-active or dispersing agent. Molded tablets may be made by molding in a suitable machine a mixture of the powdered compound moistened with an inert liquid diluent.

The tablets, and other solid dosage forms of the pharmaceutical compositions of the present invention, such as dragees, capsules, pills and granules, may optionally be scored or prepared with coatings and shells, such as enteric coatings and other coatings well known in the pharmaceutical-formulating art. They may also be formulated so as to provide slow or controlled release of the active ingredient therein using, for example, hydroxypropylmethyl cellulose in varying proportions to provide the desired release profile, other polymer matrices, liposomes and/or microspheres. They may be formulated for rapid release, e.g., freeze-dried. They may be sterilized by, for example, filtration through a bacteria-retaining filter, or by incorporating sterilizing agents in the form of sterile solid compositions which can be dissolved in sterile water, or some other sterile injectable medium immediately before use. These compositions may also optionally contain opacifying agents and may be of a composition that they release the active ingredient(s) only, or preferentially, in a certain portion of the gastrointestinal tract, optionally, in a delayed manner. Examples of embedding compositions which can be used include polymeric substances and waxes. The active ingredient can also be in micro-encapsulated form, if appropriate, with one or more of the above-described excipients.

Liquid dosage forms for oral administration of the compounds of the invention include pharmaceutically acceptable emulsions, microemulsions, solutions, suspensions, syrups and elixirs. In addition to the active ingredient, the liquid dosage forms may contain inert diluents commonly used in the art, such as, for example, water or other solvents, solubilizing agents and emulsifiers, such as ethyl alcohol, isopropyl alcohol, ethyl carbonate, ethyl acetate, benzyl alcohol, benzyl benzoate, propylene glycol, 1,3-butylene glycol, oils (in particular, cottonseed, groundnut, corn, germ, olive, castor and sesame oils), glycerol, tetrahydrofuryl alcohol, polyethylene glycols and fatty acid esters of sorbitan, and mixtures thereof.

Besides inert diluents, the oral compositions can also include adjuvants such as wetting agents, emulsifying and suspending agents, sweetening, flavoring, coloring, perfuming and preservative agents.

Suspensions, in addition to the active compounds, may contain suspending agents as, for example, ethoxylated isostearyl alcohols, polyoxyethylene sorbitol and sorbitan esters, microcrystalline cellulose, aluminum metahydroxide, bentonite, agar-agar and tragacanth, and mixtures thereof.

Formulations of the pharmaceutical compositions of the invention for rectal or vaginal administration may be presented as a suppository, which may be prepared by mixing one or more compounds of the invention with one or more suitable nonirritating excipients or carriers comprising, for example, cocoa butter, polyethylene glycol, a suppository wax or a salicylate, and which is solid at room temperature, but liquid at body temperature and, therefore, will melt in the rectum or vaginal cavity and release the active compound.

Formulations of the present invention which are suitable for vaginal administration also include pessaries, tampons, creams, gels, pastes, foams or spray formulations containing such carriers as are known in the art to be appropriate.

Dosage forms for the topical or transdermal administration of a compound of this invention include powders, sprays, ointments, pastes, creams, lotions, gels, solutions, patches and inhalants. The active compound may be mixed under sterile conditions with a pharmaceutically-acceptable carrier, and with any preservatives, buffers, or propellants which may be required.

The ointments, pastes, creams and gels may contain, in addition to an active compound of this invention, excipients, such as animal and vegetable fats, oils, waxes, paraffins, starch, tragacanth, cellulose derivatives, polyethylene glycols, silicones, bentonites, silicic acid, talc and zinc oxide, or mixtures thereof.

Powders and sprays can contain, in addition to a compound of this invention, excipients such as lactose, talc, silicic acid, aluminum hydroxide, calcium silicates and polyamide powder, or mixtures of these substances. Sprays can additionally contain customary propellants, such as chlorofluorohydrocarbons and volatile unsubstituted hydrocarbons, such as butane and propane.

Transdermal patches have the added advantage of providing controlled delivery of a compound of the present invention to the body. Such dosage forms can be made by dissolving or dispersing the compound in the proper medium. Absorption enhancers can also be used to increase the flux of the compound across the skin. The rate of such flux can be controlled by either providing a rate controlling membrane or dispersing the compound in a polymer matrix or gel.

Ophthalmic formulations, eye ointments, powders, solutions and the like, are also contemplated as being within the scope of this invention.

Pharmaceutical compositions of this invention suitable for parenteral administration comprise one or more compounds of the invention in combination with one or more pharmaceutically-acceptable sterile isotonic aqueous or nonaqueous solutions, dispersions, suspensions or emulsions, or sterile powders which may be reconstituted into sterile injectable solutions or dispersions just prior to use, which may contain sugars, alcohols, antioxidants, buffers, bacteriostats, solutes which render the formulation isotonic with the blood of the intended recipient or suspending or thickening agents.

Examples of suitable aqueous and nonaqueous carriers which may be employed in the pharmaceutical compositions of the invention include water, ethanol, polyols (such as glycerol, propylene glycol, polyethylene glycol, and the like), and suitable mixtures thereof, vegetable oils, such as olive oil, and injectable organic esters, such as ethyl oleate. Proper fluidity can be maintained, for example, by the use of coating materials, such as lecithin, by the maintenance of the required particle size in the case of dispersions, and by the use of surfactants.

These compositions may also contain adjuvants such as preservatives, wetting agents, emulsifying agents and dispersing agents. Prevention of the action of microorganisms upon the subject compounds may be ensured by the inclusion of various antibacterial and antifungal agents, for example, paraben, chlorobutanol, phenol sorbic acid, and the like. It may also be desirable to include isotonic agents, such as sugars, sodium chloride, and the like into the compositions. In addition, prolonged absorption of the injectable pharmaceutical form may be brought about by the inclusion of agents which delay absorption such as aluminum monostearate and gelatin.

In some cases, in order to prolong the effect of a drug, it is desirable to slow the absorption of the drug from subcutaneous or intramuscular injection. This may be accomplished by the use of a liquid suspension of crystalline or amorphous material having poor water solubility. The rate of absorption of the drug then depends upon its rate of dissolution which, in turn, may depend upon crystal size and crystalline form. Alternatively, delayed absorption of a parenterally-administered drug form is accomplished by dissolving or suspending the drug in an oil vehicle.

Injectable depot forms are made by forming microencapsule matrices of the subject compounds in biodegradable polymers such as polylactide-polyglycolide. Depending on the ratio of drug to polymer, and the nature of the particular polymer employed, the rate of drug release can be controlled. Examples of other biodegradable polymers include poly(orthoesters) and poly(anhydrides). Depot injectable formulations are also prepared by entrapping the drug in liposomes or microemulsions which are compatible with body tissue.

When the compounds of the present invention are administered as pharmaceuticals, to humans and animals, they can be given per se or as a pharmaceutical composition containing, for example, 0.1 to 99% (more preferably, 10 to 30%) of active ingredient in combination with a pharmaceutically acceptable carrier.

The preparations of the present invention may be given orally, parenterally, topically, or rectally. They are of course given in forms suitable for each administration route. For example, they are administered in tablets or capsule form, by injection, inhalation, eye lotion, ointment, suppository, etc. administration by injection, infusion or inhalation; topical by lotion or ointment; and rectal by suppositories. Oral administrations are preferred.

The phrases “parenteral administration” and “administered parenterally” as used herein means modes of administration other than enteral and topical administration, usually by injection, and includes, without limitation, intravenous, intramuscular, intraarterial, intrathecal, intracapsular, intraorbital, intracardiac, intradermal, intraperitoneal, transtracheal, subcutaneous, subcuticular, intraarticular, subcapsular, subarachnoid, intraspinal and intrasternal injection and infusion.

The phrases “systemic administration,” “administered systemically,” “peripheral administration” and “administered peripherally” as used herein mean the administration of a compound, drug or other material other than directly into the central nervous system, such that it enters the patient's system and, thus, is subject to metabolism and other like processes, for example, subcutaneous administration.

These compounds may be administered to humans and other animals for therapy by any suitable route of administration, including orally, nasally, as by, for example, a spray, rectally, intravaginally, parenterally, intracisternally and topically, as by powders, ointments or drops, including buccally and sublingually.

Regardless of the route of administration selected, the compounds of the present invention, which may be used in a suitable hydrated form, and/or the pharmaceutical compositions of the present invention, are formulated into pharmaceutically-acceptable dosage forms by conventional methods known to those of skill in the art.

Actual dosage levels of the active ingredients in the pharmaceutical compositions of this invention may be varied so as to obtain an amount of the active ingredient which is effective to achieve the desired therapeutic response for a particular patient, composition, and mode of administration, without being toxic to the patient.

The selected dosage level will depend upon a variety of factors including the activity of the particular compound of the present invention employed, or the ester, salt or amide thereof, the route of administration, the time of administration, the rate of excretion or metabolism of the particular compound being employed, the rate and extent of absorption, the duration of the treatment, other drugs, compounds and/or materials used in combination with the particular compound employed, the age, sex, weight, condition, general health and prior medical history of the patient being treated, and like factors well known in the medical arts.

A physician or veterinarian having ordinary skill in the art can readily determine and prescribe the effective amount of the pharmaceutical composition required. For example, the physician or veterinarian could start doses of the compounds of the invention employed in the pharmaceutical composition at levels lower than that required in order to achieve the desired therapeutic effect and gradually increase the dosage until the desired effect is achieved.

In general, a suitable daily dose of a compound of the invention will be that amount of the compound which is the lowest dose effective to produce a therapeutic effect. Such an effective dose will generally depend upon the factors described above. Preferably, the compounds are administered at about 0.01 mg/kg to about 200 mg/kg, more preferably at about 0.1 mg/kg to about 100 mg/kg, even more preferably at about 0.5 mg/kg to about 50 mg/kg. When the compounds described herein are co-administered with another agent (e.g., as sensitizing agents), the effective amount may be less than when the agent is used alone.

If desired, the effective daily dose of the active compound may be administered as two, three, four, five, six or more sub-doses administered separately at appropriate intervals throughout the day, optionally, in unit dosage forms. Preferred dosing is one administration per day.

The invention further provides a unit dosage form (such as a tablet or capsule) comprising a compound described herein in a therapeutically effective amount for the treatment of a medical disorder described herein.

### EXAMPLES

The invention now being generally described, will be more readily understood by reference to the following examples, which are included merely for purposes of illustration of certain aspects and embodiments of the present invention, and is not intended to limit the invention.

### Abbreviations

¹H NMR Proton nuclear magnetic resonance spectroscopy
ACN Acetonitrile
AcOH Acetic acid
aq. aqueous
Ar Argon (gas)
BINAP 2,2′-bis(diphenylphosphino)-1,1′-binaphthyl
BnBr Benzyl bromide
Boc₂O Di-tert-butyl dicarbonate
calcd. calculated
CDCl₃ Deuterated chloroform
Cs₂CO₃ Caesium carbonate
DCE 1,2-Dichloroethane
DCM Dichloromethane
DIEA N,N-Diisopropylethylamine
DMA Dimethylacetamide
DMF N,N-Dimethylformamide
DMSO Dimethylsulfoxide
DMSO-d₆ Hexadeuterodimethyl sulfoxide
EA Ethyl acetate
EDCI 1-(3-Dimethylaminopropyl)-3-ethylcarbodiimide hydrochloride
EtOAc Ethyl acetate
EtOH Ethanol
Fe Iron
H₂ Hydrogen (gas)
HATU N-[(dimethylamino)-3-oxo-1H-1,2,3-triazolo[4,5-b]pyridin-1-yl-methylene]-N-methylmethanaminium hexafluorophosphate
HCl Hydrochloric acid
H₂O Water
H₂O₂ Hydrogen peroxide
HOAc Acetic acid
HPLC High performance liquid chromatography
hr hour
hrs hours
i-PrMgCl Isopropylmagnesium chloride
KF Potassium fluoride
LCMS Liquid Chromatography Mass Spectrometry
LDA Lithium diisopropylamide
LiCl Lithium chloride
LiOH Lithium hydroxide
m-CPBA 3-Chloroperbenzoic acid
MeI Iodomethane
MeOD Tetradeuteromethanol
MeOH Methanol
min minute(s)
MnO₂ Manganese dioxide
MS Mass spectrometry
N₂ Nitrogen (gas)
NaBH(OAc): Sodium triacetoxyborohydride
NaH Sodium hydride
NaHCO₃Sodium bicarbonate
NaOH Sodium hydroxide
Na₂CO₃ Sodium carbonate
Na₂SO₄ Sodium sulfate
Na₂S₂O₃ Sodium thiosulfate
Na₂S₂O₄ Sodium dithionite
NBS N-Bromosuccinimide
NH₄Cl Ammonium chloride
NH₄HCO₃ Ammonium bicarbonate
O₂ Oxygen (gas)
PBr₃ Phosphorus tribromide
Pd Palladium
Pd₂(dba)₃ Tris(dibenzylideneacetone) dipalladium
Pd/C Palladium on carbon
Pd(dppf)Cl₂ [1,1′-Bis(diphenylphosphino) ferrocene]dichloropalladium (II)
Pd(OAc)₂ Palladium(II) acetate
PE Petroleum ether
PPh₃ Triphenylphosphine
prep-HPLC Preparative high performance liquid chromatography
psi Pound per square inch
py Pyridine
RT Room temperature
Rₜ Retention time
sat. saturated
SFC Supercritical fluid chromatography
SOCl₂ Thionyl chloride
t-BuONa Sodium tert-butoxide
TEA Triethylamine
TFA Trifluoroacetic acid
THF Tetrahydrofuran
TMSCl Trimethylsilyl chloride
Zn Zinc

### Example 1. Preparation of N-(4-(2-(2-aminopyridin-3-yl)-5-phenyl-3H-imidazo[4,5-b]pyridin-3-yl)benzyl)-4-hydroxybenzamide (Compound 1)

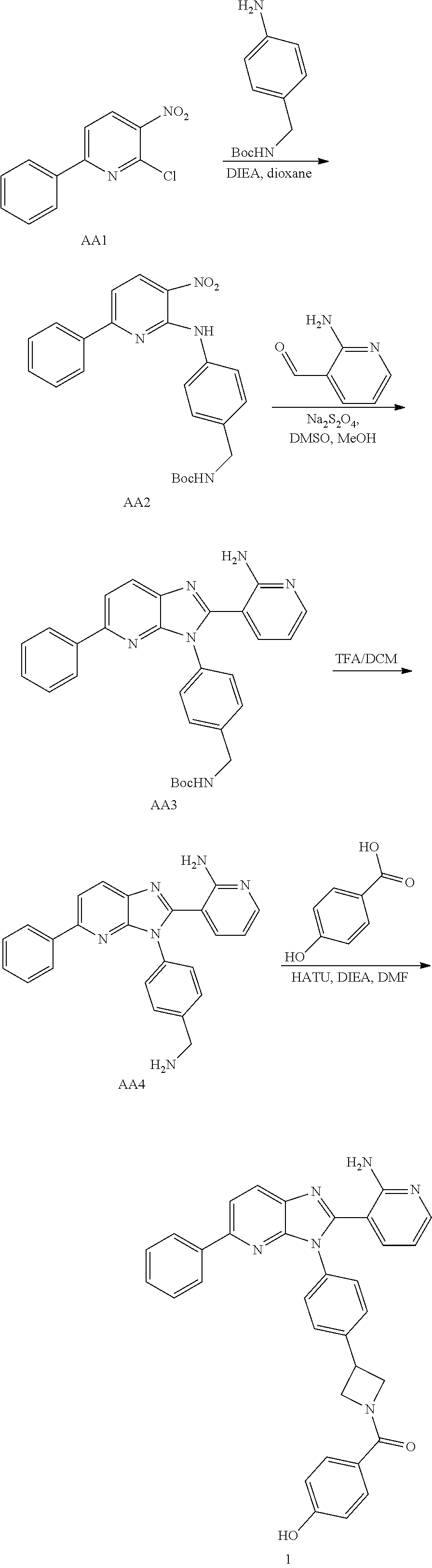

Step 1. To a solution of 2-chloro-3-nitro-6-phenyl-pyridine AA1 (15.0 g, 63.9 mmol) and tert-butyl N-[(4-aminophenyl)methyl]carbamate (19.2 g, 86.3 mmol) in 1,4-dioxane (50.0 mL), DIEA (32.0 mL, 192 mmol) was added and the mixture stirred at 80° C. for 12 hrs. Water (20 mL) was added to the reaction mixture and the resulting mixture was extracted with EtOAc (30.0 mL×3). The combined organic phase was washed with brine (30 mL×3), dried over anhydrous Na₂SO₄, filtered and concentrated. The residue was purified by flash silica gel chromatography (eluent with 0-30% of EtOAc in PE) to afford tert-butyl N-[4-[(3-nitro-6-phenyl-2-pyridyl)amino]phenyl]methyl]carbamate AA2 (26.0 g, 61.8 mmol, 96.7% yield) as a yellow solid. ¹H NMR (DMSO-d₆, 400 MHz) δ_{H}=10.09 (s, 1H), 8.60 (d, J=8.8 Hz, 1H), 8.11-8.07 (m, 2H), 7.69 (d, J=8.4 Hz, 2H), 7.58 (d, J=8.8 Hz, 1H), 7.55-7.50 (m, 3H), 7.29 (d, J=8.4 Hz, 2H), 4.15 (d, J=6.0 Hz, 2H), 1.41 (m, 9H).

Step 2. To a solution of tert-butyl N-[4-[(3-nitro-6-phenyl-2-pyridyl)amino]phenyl methyl]carbamate AA2 (30.0 g, 71.3 mmol) and 2-aminopyridine-3-carbaldehyde (11.8 g, 96.3 mmol) in DMSO (100 mL) and methanol (100 mL), Na₂S₂O₄ (37.3 g, 214 mmol) was added under N₂, and the mixture stirred at 25° C. for 12 hrs. The reaction mixture was concentrated, aq. LiCl (30 mL) was added to the residue, and the resulting mixture was extracted with EtOAc (30 mL×3). The combined organic phase was washed with brine (30 mL), dried over anhydrous Na₂SO₄, filtered and concentrated. The residue was purified by flash silica gel chromatography (eluent with 0-5% of MeOH in DCM) to afford tert-butyl N-[[4-[2-(2-amino-3-pyridyl)-5-phenyl-imidazo[4,5-b]pyridin-3-yl]phenyl]methyl]carbamate AA3 (5.90 g, 11.4 mmol, 15.9% yield) as a yellow solid. ¹H NMR (DMSO-d₆, 400 MHz) δ_{H}=8.26 (d, J=8.4 Hz, 1H), 8.04-7.97 (m, 4H), 7.47-7.39 (m, 7H), 7.22-7.18 (m, 1H), 6.99 (s, 2H), 6.44-6.37 (m, 1H), 4.28-4.20 (m, 2H), 1.41 (s, 9H).

Step 3. To a solution of tert-butyl N-[[4-[2-(2-amino-3-pyridyl)-5-phenyl-imidazo[4,5-b]pyridin-3-yl]phenyl]methyl]carbamate AA3 (100 mg, 0.203 mmol) in DCM (2.50 mL), TFA (0.500 mL) was added and the mixture stirred at 25° C. for 12 hrs. The reaction mixture was concentrated directly. The residue was purified by prep-HPLC (Column: Welch Xtimate C18 150*30 mm*5 μm; mobile phase: [water (0.225% FA)-ACN]; B %: 0%-26%; 36 min) to afford 3-(3-(4-(aminomethyl)phenyl)-5-phenyl-3H-imidazo[4,5-b]pyridin-2-yl)pyridin-2-amine AA4 (19.1 mg, 0.0487 mmol, 24.0% yield) as a light yellow solid. ¹H NMR (METHANOL-d₄, 400 MHz) δ_{H}=8.21 (d, J=8.4 Hz, 1H), 8.04-7.93 (m, 4H), 7.68-7.57 (m, 4H), 7.45-7.35 (m, 4H), 6.48 (dd, J=5.2, 7.6 Hz, 1H), 4.22 (s, 2H). HPLC Rₜ=3.920 min in 8 min chromatography, purity 100%. LCMS Rₜ=2.005 min in 4 min chromatography, purity 99.5%, MS ESI calcd, for 392.17, [M+H]⁺ 393.17, found 393.4.

Step 4. To a solution of 3-(3-(4-(aminomethyl)phenyl)-5-phenyl-3H-imidazo[4,5-b]pyridin-2-yl)pyridin-2-amine AA4 (100 mg, 0.255 mmol) and 4-hydroxybenzoic acid (35.0 mg, 0.255 mmol) in DMF (5.00 mL), DIEA (99.0 mg, 0.764 mmol) and HATU (145 mg, 0.382 mmol) were added, and the mixture stirred at 25° C. for 2 hrs. Water (2 mL) was added to the reaction mixture and the resulting mixture was extracted with EtOAc (5 mL×3). The combined organic phase was washed with brine (10 mL), dried over anhydrous Na₂SO₄, filtered and concentrated. The residue was purified by prep-HPLC (Column: Welch Xtimate C18 150*30 mm*5 μm; mobile phase: [water (FA)-ACN]; B %: 26%-66%; 20 min) to afford N-(4-(2-(2-aminopyridin-3-yl)-5-phenyl-3H-imidazo[4,5-b]pyridin-3-yl)benzyl)-4-hydroxybenzamide 1 (10.4 mg, 0.0198 mmol, 7.78% yield) as a light yellow solid. ¹H NMR (DMSO-d₆, 400 MHz) δ_{H}=9.99 (s, 1H), 8.89 (t. J=6.0 Hz, 1H), 8.26 (d, J=8.4 Hz, 1H), 8.04-7.97 (m, 4H), 7.80 (d, J=8.8 Hz, 2H), 7.49-7.43 (m, 6H), 7.42-7.36 (m, 1H), 7.26-7.20 (m, 1H), 6.99 (s, 2H), 6.82 (d, J=8.4 Hz, 2H), 6.43 (dd, J=4.8, 7.6 Hz, 1H), 4.57 (d, J=6.0 Hz, 2H). HPLC Rₜ=3.452 min in 8 min chromatography, purity 97.7%. LCMS Rₜ=1.182 min in 2 min chromatography, purity 97.4%, MS ESI calcd, for 512.20, [M+H]⁺ 513.20, found 513.0.

### Example 2. Synthesis of N-(4-(2-(2-aminopyridin-3-yl)-5-phenyl-3H-imidazo[4,5-b]pyridin-3-yl)benzyl)-2-hydroxybenzamide (Compound 2)

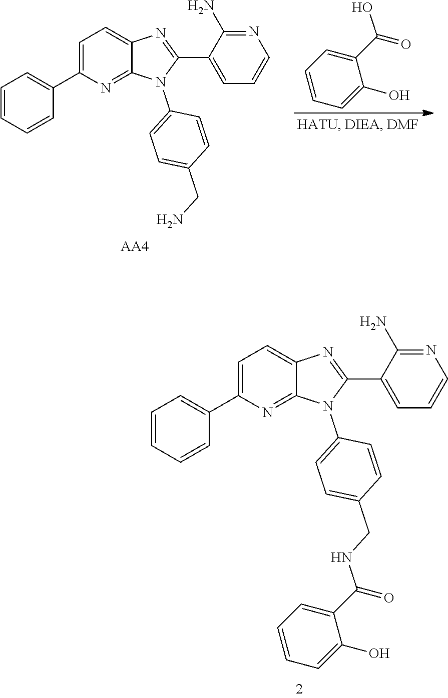

Step 1. To a solution of 3-(3-(4-(aminomethyl)phenyl)-5-phenyl-3H-imidazo[4,5-b]pyridin-2-yl)pyridin-2-amine AA4 (100 mg, 0.255 mmol) and 2-hydroxybenzoic acid (35.0 mg, 0.255 mmol) in DMF (5.00 mL), DIEA (99.0 mg, 0.764 mmol) and HATU (145 mg, 0.382 mmol) were added and the mixture stirred at 25° C. for 2 hrs. Water (2 mL) was added to the reaction mixture and the resulting mixture was extracted with EtOAc (5 ml×3). The combined organic phase was washed with brine (10 mL), dried over anhydrous Na₂SO₄, filtered and concentrated. The residue was purified by prep-HPLC (Column: Welch Xtimate C18 150*30 mm*5 μm; mobile phase: [water (FA)-ACN]; B %: 26%-66%; 20 min) to afford N-(4-(2-(2-aminopyridin-3-yl)-5-phenyl-3H-imidazo[4,5-b]pyridin-3-yl)benzyl)-2-hydroxybenzamide 2 (18.7 mg, 0.0365 mmol, 14.3% yield) as a light yellow solid. ¹H NMR (DMSO-d₆, 400 MHz) δ_{H}=12.47 (s, 1H), 9.49-9.39 (m, 1H), 8.26 (d, J=8.4 Hz, 1H), 8.05-7.91 (m, 5H), 7.52-7.36 (m, 8H), 7.26-7.20 (m, 1H), 7.01-6.88 (m, 4H), 6.42 (dd, J=4.8, 7.6 Hz, 1H), 4.65 (d, J=6.0 Hz, 2H). HPLC R: =3.850 min in 8 min chromatography, purity 99.9%. LCMS Rₜ=1.426 min in 2 min chromatography, purity 98.4%, MS ESI calcd, for 512.20. [M+H]⁺ 513.20, found 512.8.

### Example 3. Synthesis of N-(4-(2-(2-aminopyridin-3-yl)-5-phenyl-3H-imidazo[4,5-b]pyridin-3-yl)benzyl)-3,4-dihydroxybenzamide (Compound 3)

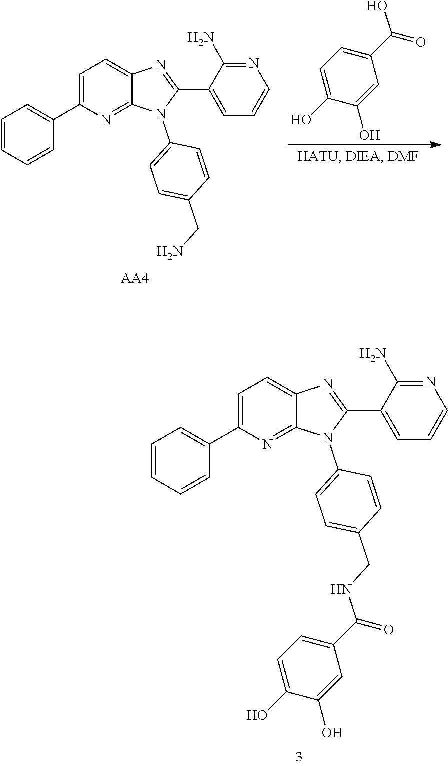

Step 1. To a solution of 3-(3-(4-(aminomethyl)phenyl)-5-phenyl-3H-imidazo[4,5-b]pyridin-2-yl)pyridin-2-amine AA4 (100 mg, 0.255 mmol) and 3,4-dihydroxybenzoic acid (39.0 mg, 0.255 mmol) in DMF (5.00 mL), DIEA (99.0 mg, 0.764 mmol) and HATU (145 mg, 0.382 mmol) were added and the mixture stirred at 25° C. for 2 hrs. Water (2 mL) was added to the reaction mixture, and the resulting mixture was extracted with EtOAc (5 mL×3). The combined organic phase was washed with brine (10 mL), dried over anhydrous Na₂SO₄, filtered and concentrated. The residue was purified by prep-HPLC (Column: Welch Xtimate C18 150*30 mm*5 μm; mobile phase: [water (NH₃H₂O+NH₄HCO₃)-ACN]; B %: 26%-66%; 20 min) to afford N-(4-(2-(2-aminopyridin-3-yl)-5-phenyl-3H-imidazo[4,5-b]pyridin-3-yl)benzyl)-3,4-dihydroxybenzamide 3 (1.8 mg, 0.00336 mmol, 1.32% yield) as a light yellow solid. 1H NMR (DMSO-d₆, 400 MHz) δ_{H}=8.86-8.77 (m, 1H), 8.26 (d, J=8.4 Hz, 1H), 8.04-7.96 (m, 4H), 7.49-7.34 (m, 9H), 7.30-7.26 (m, 1H), 7.23-7.19 (m, 1H), 6.94 (s, 2H), 6.77 (d, J=8.4 Hz, 1H), 6.42 (dd, J=4.8, 7.6 Hz, 1H), 4.58-4.50 (m, 2H). HPLC Rₜ=2.484 min in 8 min chromatography, purity 98.5%. LCMS Rₜ=1.159 min in 2 min chromatography, purity 97.3%, MS ESI calcd, for 528.19, [M+H]⁺ 529.19, found 528.8.

### Example 4. Synthesis of N-(4-(2-(2-aminopyridin-3-yl)-5-phenyl-3H-imidazo[4,5-b]pyridin-3-yl)benzyl)-4-hydroxy-3-methoxybenzamide (Compound 4)

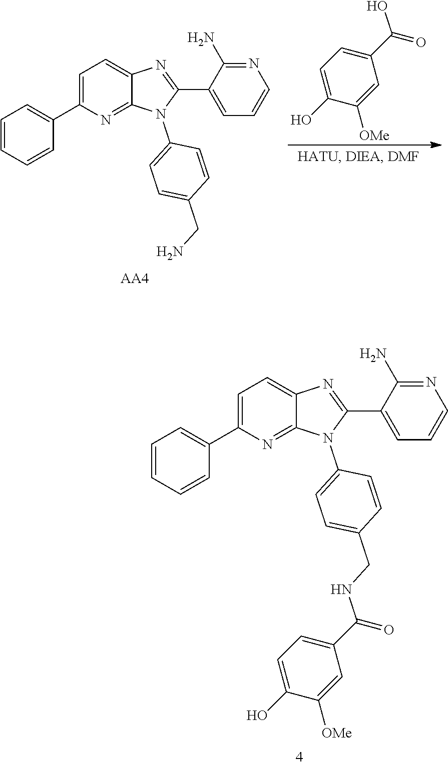

Step 1. To a solution of 3-(3-(4-(aminomethyl)phenyl)-5-phenyl-3H-imidazo[4,5-b]pyridin-2-yl)pyridin-2-amine AA4 (100 mg, 0.255 mmol) and 4-hydroxy-3-methoxy-benzoic acid (43.0 mg, 0.255 mmol) in DMF (5.00 mL), DIEA (99.0 mg, 0.764 mmol) and HATU (145 mg, 0.382 mmol) were added and the mixture stirred at 25° C. for 2 hrs. Water (2 mL) was added to the reaction mixture and the resulting mixture was extracted with EtOAc (5 mL×3). The combined organic phase was washed with brine (10 mL), dried over anhydrous Na₂SO₄, filtered and concentrated. The residue was purified by prep-HPLC (Column: Welch Xtimate C18 150*30 mm*5 μm; mobile phase: [water (NH₃H₂O+NH₄HCO₃)-CAN]: B %: 26%-66%; 20 min) to afford N-(4-(2-(2-aminopyridin-3-yl)-5-phenyl-3H-imidazo[4,5-b]pyridin-3-yl)benzyl)-4-hydroxy-3-methoxy benzamide 4 (11.3 mg, 0.0204 mmol, 8.01% yield) as a light yellow solid. ¹H NMR (DMSO-d₆, 400 MHz) δ_{H}=9.58 (s, 1H), 8.93 (t, J=6.0 Hz, 1H), 8.28 (d, J=8.4 Hz, 1H), 8.05-7.97 (m, 4H), 7.53-7.22 (m. 12H), 6.83 (d, J=8.4 Hz, 1H), 6.54 (dd, J=5.2, 7.6 Hz, 1H), 4.58 (d, J=6.0 Hz, 2H), 3.82 (s, 3H). HPLC Rₜ=2.677 min in 8 min chromatography, purity 98.0%. LCMS Rₜ=1.218 min in 2 min chromatography, purity 96.9%, MS ESI calcd. For 542.21, [M+H]⁺ 543.21, found 542.9.

### Example 5. Synthesis of N-[[4-[2-(2-amino-3-pyridyl)-5-phenyl-imidazo[4,5-b]pyridin-3-yl]phenyl]methyl]-4-fluoro-3-hydroxy-benzamide (Compound 5)

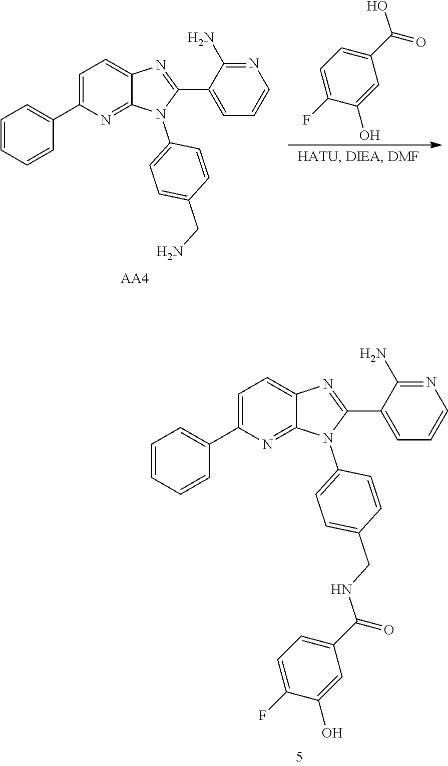

Step 1. To the solution of 3-(3-(4-(aminomethyl)phenyl)-5-phenyl-3H-imidazo[4,5-b]pyridin-2-yl)pyridin-2-amine AA4 (151 mg, 0.384 mmol) in DMF (2.00 mL), HATU (175 mg. 0.461 mmol) and DIEA (0.210 mL, 1.15 mmol) were added. After stirring for 30 mins, 4-fluoro-3-hydroxy-benzoic acid (60.0 mg, 0.384 mmol) was added, and the reaction mixture was stirred for 12 hrs. The reaction mixture was purified directly by HPLC Welch Xtimate C18 150*25 mm*5 μm, water (NH₄HCO₃)-CAN C18 150×30 mm water (NH₃H₂O+NH₄HCO₃)-CAN to give N-[[4-[2-(2-amino-3-pyridyl)-5-phenyl-imidazo[4,5-b]pyridin-3-yl]phenyl methyl]-4-fluoro-3-hydroxy-benzamide 5 (30.0 mg, 0.0510 mmol, 13.3% yield) as a white solid. ¹H NMR (400 MHz, DMSO-d₆) δ_{H}=10.20-10.01 (m, 1H), 9.07 (t, J=5.2 Hz, 1H), 8.26 (d, J=8.4 Hz, 1H), 8.06-7.96 (m, 4H), 7.51-7.56 (m, 1H), 7.50-7.43 (m, 6H), 7.41-7.36 (m, 2H) 7.26-7.19 (m, 2H), 6.97-6.91 (m, 2H) 6.42 (dd, J=8.0, 4.8 Hz, 1H), 4.62-4.53 (m, 2H). HPLC Rₜ=5.109 min in 8 min chromatography, purity 90.5%. LCMS Rₜ=2.659 min in 4 min chromatography, purity 87.4%, MS ESI calcd. For 530.19 [M+H]+ 531.19, found 531.1.

### Example 6. Synthesis of N-(4-(2-(2-aminopyridin-3-yl)-5-phenyl-3H-imidazo[4,5-b]pyridin-3-yl)benzyl)-2,4-difluoro-3-hydroxybenzamide (Compound 6)

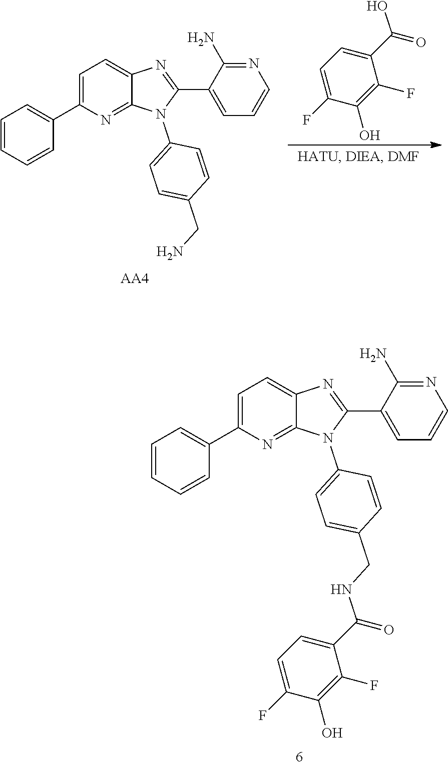

Step 1. To a solution of 2,4-difluoro-3-hydroxy-benzoic acid (44.0 mg, 0.255 mmol) in DMF (4.00 mL), DIEA (99.0 mg, 0.764 mmol), HATU (145 mg, 0.382 mmol) and 3-(3-(4-(aminomethyl)phenyl)-5-phenyl-3H-imidazo[4,5-b]pyridin-2-yl)pyridin-2-amine AA4 (100 mg, 0.255 mmol) were added at 25° C., and the mixture stirred at 25° C. for 16 hrs. Water (20 mL) was added, and the resulting mixture was extracted with EtOAc (10 ml×3). The combined organic phase was washed with brine (5 mL), water (5 mL), dried over anhydrous Na₂SO₄, filtered and concentrated. The residue was purified by prep-HPLC (column: Xtimate C18 150*40 mm*10 μm; condition: water (NH₄HCO₃)-ACN; begin b: 8-48) to give N-(4-(2-(2-aminopyridin-3-yl)-5-phenyl-3H-imidazo[4,5-b]pyridin-3-yl)benzyl)-2,4-difluoro-3-hydroxybenzamide 6 (35.0 mg, 23.1% yield) as a yellow solid. ¹H NMR (400 MHz, DMSO-d₆) δ=10.66-10.21 (m, 1H), 8.94 (t, J=5.6 Hz, 1H), 8.27 (d, J=8.4 Hz, 1H), 8.07-7.95 (m, 4H), 7.52-7.36 (m, 7H), 7.22 (dd, J=7.6, 2.0 Hz, 1H), 7.11 (d, J=8.4 Hz, 2H), 6.95 (s, 2H), 6.42 (dd, J=7.6, 4.8 Hz, 1H), 4.57 (d, J=6.0 Hz, 2H). HPLC R: =2.677 min in 8 min chromatography, purity 92.4%. LCMS Rₜ=1.231 min in 2 min chromatography, purity 93.4%, MS ESI calcd, for 548.18, [M+H]⁺ 549.18, found 549.0.

### Example 7. Synthesis of 4-((4-(2-(2-aminopyridin-3-yl)-5-phenyl-3H-imidazo[4,5-b]pyridin-3-yl)benzyl)carbamoyl)benzenesulfonic acid (Compound 7)

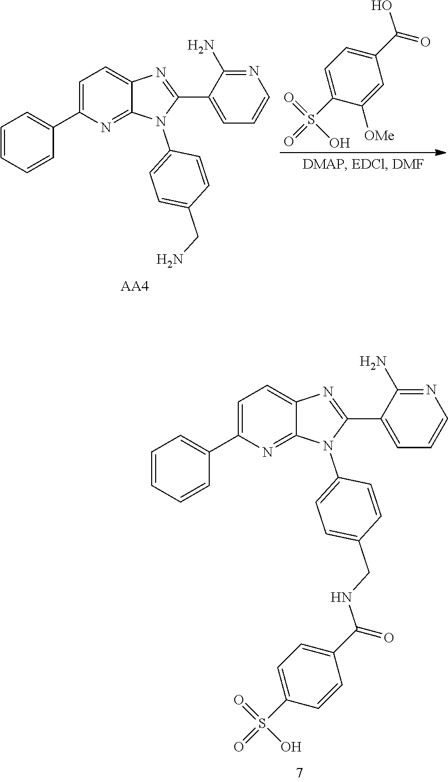

Step 1. To a solution of 4-sulfobenzoic acid (103 mg, 0.510 mmol) in DMF (5.00 mL), DMAP (62.0 mg, 0.510 mmol) and EDCI (147 mg, 0.764 mmol) were added. The mixture was degassed, purged with N₂ (>3), and stirred at 0° C. for 0.25 hrs. 3-(3-(4-(Aminomethyl)phenyl)-5-phenyl-3H-imidazo[4,5-b]pyridin-2-yl)pyridin-2-amine AA4 (200 mg, 0.510 mmol) was then added to the mixture and the mixture stirred at 25° C. for 24 hrs. The reaction mixture was poured into water (30 mL), and the resulting mixture was extracted with BIOAc (20 mL×3). The combined organic phase was washed with brine (30 ml×5), dried over anhydrous Na₂SO₄ and concentrated. The crude product was purified by prep-HPLC (column: welch xtimate c18 150*40 mm*10 μm; mobile phase: [water (HCl)-ACN]; B %: 6%-46%: 36 min) to give an impure product. The impure product was purified by prep-HPLC (column: welch xtimate c18 150*40 mm*10 μm; mobile phase: [water (HCl)-ACN]; B %: 14%-54%: 36 min) to give 4-((4-(2-(2-aminopyridin-3-yl)-5-phenyl-3H-imidazo[4,5-b]pyridin-3-yl)benzyl)carbamoyl)benzenesulfonic acid 7 (29.5 mg, 39.9% yield) as a light yellow solid. ¹H NMR (400 MHz, DMSO-d₆) δ_{H}=9.17 (t, J=6.0 Hz, 1H), 8.45-8.27 (m, 2H), 8.12-8.01 (m, 4H), 7.92 (dd, J=1.6, 7.6 Hz, 1H), 7.90-7.86 (m, 2H), 7.72-7.66 (m, 2H), 7.58-7.46 (m, 6H), 7.44-7.38 (m, 1H), 6.90 (dd, J=6.4, 7.6 Hz, 1H), 4.60 (d, J=5.6 Hz, 2H). HPLC Rₜ=2.416 min in 8 min chromatography, purity 99.3%. LCMS Rₜ=2.258 min in 4 min; Agilent PoroShell 120 EC-C18 2.7 μm 3.0*50 mm; purity 91.0%, MS ESI calcd, for 576.16, [M+H]⁺ 577.16, found 577.1.

### Example 8. Synthesis of 3-((4-(2-(2-aminopyridin-3-yl)-5-phenyl-3H-imidazo[4,5-b]pyridin-3-yl)benzyl)carbamoyl)benzenesulfonic acid (Compound 8)

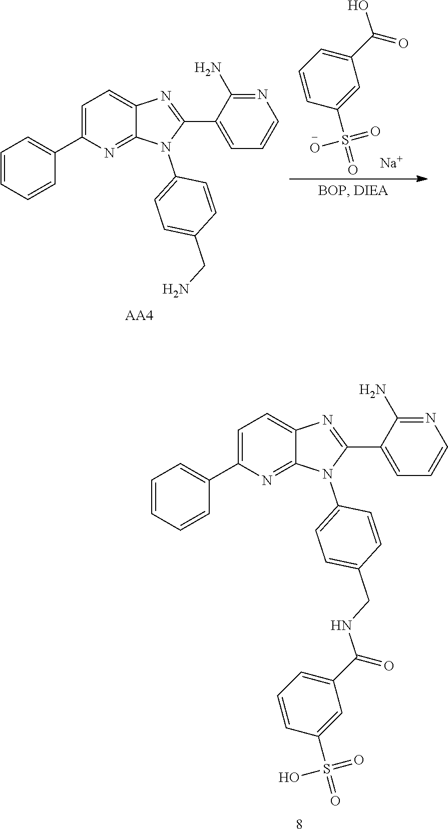

Step 1. To a solution of 3-(3-(4-(aminomethyl)phenyl)-5-phenyl-3H-imidazo[4,5-b]pyridin-2-yl)pyridin-2-amine AA4 (100 mg, 0.250 mmol) in DMF (3.00 mL), sodium 3-sulfobenzoate (52.0 mg, 0.250 mmol) and DIEA (99.0 mg, 0.760 mmol) were added. BOP (146 mg. 0.330 mmol) was then added to the mixture, and the mixture was allowed to stir at 25° C. for 12 hrs. The reaction mixture was diluted with DMF (5 mL) and the crude material was purified by prep-HPLC (column: welch xtimate C18 150*40 mm*10 μm; mobile phase: [water (HCl)-ACN]: B %: 6%-46%: 36 min) to give 3-(4-(2-(2-aminopyridin-3-yl)-5-phenyl-3H-imidazo[4,5-b]pyridin-3-yl)benzyl)carbamoyl)benzenesulfonic acid 8 (11.7 mg, 7.8% yield) as a yellow solid. ¹H NMR (400 MHz, DMSO-d₆) δ_{H}=9.41-9.15 (m, 1H), 8.32 (d, J=8.4 Hz, 1H), 8.18 (s, 1H), 8.09-8.00 (m, 4H), 7.89-7.80 (m, 2H), 7.79-7.72 (m, 1H), 7.59-7.33 (m, 8H), 6.91-6.81 (m, 1H), 4.58 (d, J=5.6 Hz, 2H). HPLC Rₜ=2.193 min in 8 min chromatography, purity 99.8%. LCMS Rₜ=2.076 min in 4 min; Agilent PoroShell 120 EC-C18 2.7 μm 3.0*50 mm; purity 99.4%, MS ESI calcd, for 576.16, [M+H]⁺ 577.16, found 577.1.

### Example 9. Synthesis of 3-((4-(2-(2-aminopyridin-3-yl)-5-phenyl-3H-imidazo[4,5-b]pyridin-3-yl)benzyl)carbamoyl)-2-fluorobenzenesulfonic acid (Compound 9)

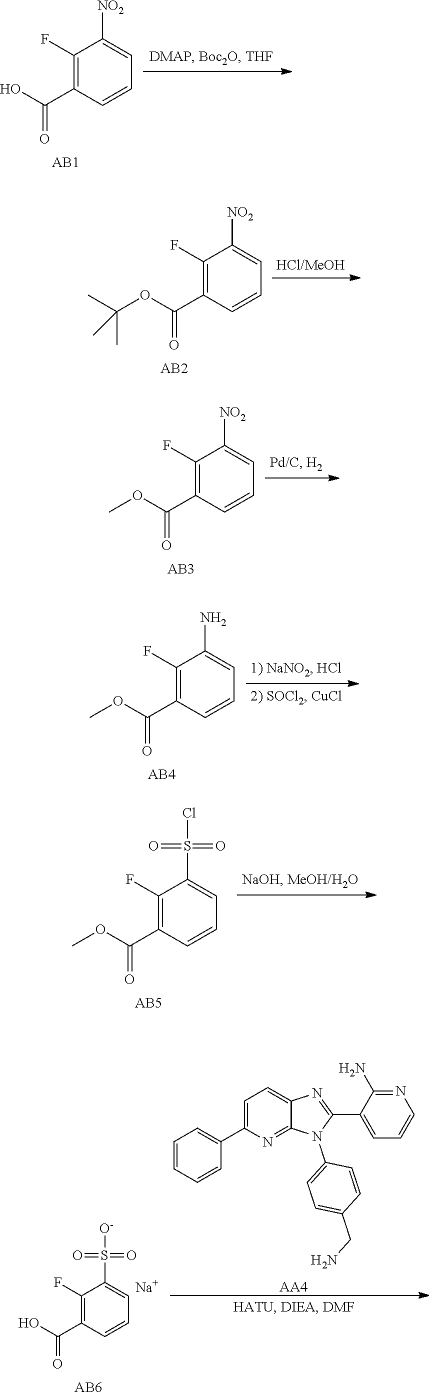

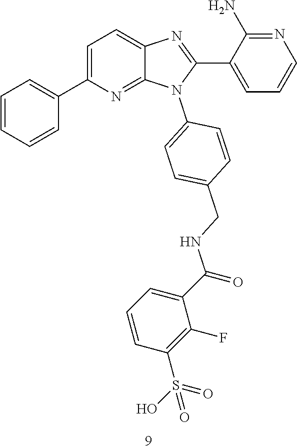

Step 1. A mixture of 2-fluoro-3-nitro-benzoic acid AB1 (5.00 g, 27.0 mmol), Boc₂O (11.8 g, 54.0 mmol) and DMAP (990 mg, 8.10 mmol) in THF (50.0 mL) was degassed and purged with N₂ (>3), and the mixture was stirred at 35° C. for 12 hrs under N₂ atmosphere. The mixture was diluted with H₂O (50 mL), extracted with EtOAc (100 mL×3). The organic layer was dried over Na₂SO₄, concentrated, and the residue was purified by flash silica gel chromatography (ISCOR: 80 g SepaFlash® Silica Flash Column, Eluent of 4% Ethyl acetate/Petroleum ether gradient @ 100 mL/min) to give tert-butyl 2-fluoro-3-nitro-benzoate AB2 (3.70 g, 56.8% yield) as a colorless liquid. ¹H NMR (400 MHz, DMSO-d₆) δ_{H}=8.33 (d, J=6.8 Hz, 1H), 8.15 (d, J=6.8 Hz, 1H), 7.52 (d, J=8.0 Hz, 1H), 1.55 (s, 9H).

Step 2. A mixture of tert-butyl 2-fluoro-3-nitro-benzoate AB2 (3.70 g, 13.3 mmol) in HCl/MeOH (40.0 mL) was stirred at 25° C. for 12 hrs. The reaction mixture was concentrated directly to give methyl 2-fluoro-3-nitro-benzoate AB3 (3.03 g, crude) as a liquid. ¹H NMR (400 MHz, DMSO-d₆) δ_{H}=8.39-8.35 (m, 1H), 8.25-8.21 (m, 1H), 7.55 (d, J=8.0 Hz, 1H), 3.90 (s, 3H).

Step 3. To a solution of methyl 2-fluoro-3-nitro-benzoate AB3 (3.03 g, 15.2 mmol) in methanol (40.0 mL), Pd/C (500 mg, 10%, wet) was added under a N₂ atmosphere. The suspension was degassed and purged with H₂ (×3), and the reaction mixture was subsequently stirred under H₂ (15 psi) at 25° C. for 12 hrs. The mixture was filtered via a celite pad and the filter cake was washed with DCM/MeOH=1/1 (600 mL). The organic phase was concentrated to give methyl 3-amino-2-fluorobenzoate AB4 (2.34 g, crude) as a yellow solid. ¹H NMR (400 MHz, DMSO-d₆) δ_{H}=7.03-6.92 (m, 3H), 4.89 (s, 2H), 3.81 (s, 3H).

Step 4. A solution of SOCl₂ (225 mg, 1.89 mmol) in water (2.16 mL) was prepared at 0° C., and the mixture was subsequently allowed to stir at 25° C. for 1 hr. CuCl (29.0 mg, 0.290 mmol) was then added at 0° C. over 15 mins to provide a solution A. To a solution of HCl (2.20 mL) in water (6.48 mL), methyl 3-amino-2-fluorobenzoate AB4 (500 mg, 2.96 mmol) was added at 0° C. NaNO₂ (408 mg, 5.91 mmol) was then added, and the resulting mixture was stirred at 0° C. for 15 mins to give a solution B. Solution A was added into solution B, dropwise, and the resulting mixture was stirred at 0° C. for 2 hrs. The mixture was diluted with H₂O (10 mL), extracted with DCM (10 mL×3). The organic layer was dried over Na₂SO₄, filtered and the filtrate was concentrated to give methyl 3-(chlorosulfonyl)-2-fluorobenzoate AB5 (569 mg, 76.3% yield) as a black brown liquid.

Step 5. To a solution of methyl 3-(chlorosulfonyl)-2-fluorobenzoate AB5 (369 mg, 1.46 mmol) in water (3.00 mL), NaOH (292 mg, 7.31 mmol), methanol (3.00 mL), and THF (2.00 mL) were added. After addition, the mixture was stirred at 25° C. for 3 hrs. The reaction mixture was concentrated directly to give sodium 3-carboxy-2-fluorobenzenesulfonate AB6 (500 mg, crude) as a black brown solid.

Step 6. To a solution of 3-(3-(4-(aminomethyl)phenyl)-5-phenyl-3H-imidazo[4,5-b]pyridin-2-yl)pyridin-2-amine AA4 (700 mg, 1.78 mmol) in DMF (3.00 mL), 2-fluoro-3-sulfo-benzoic acid AB6 (500 mg, 2.28 mmol) and DIEA (1.15 g, 8.92 mmol) were added. After addition, the mixture was stirred at 25° C. for 5 mins, HATU (1.35 g, 3.57 mmol) was then added, and the resulting mixture further stirred at 25° C. for 2 hrs. The mixture was diluted with H₂O (10 mL), extracted with EtOAc (10 mL×3), washed with brine (30 mL). The organic layer was dried over Na₂SO₄, filtered and concentrated. The crude product was purified by prep-HPLC (column: Xtimate C18 150*40 mm*10 μm; mobile phase: water (HCl)-ACN; B %: 12%-52%, 36 min) to give 3-((4-(2-(2-aminopyridin-3-yl)-5-phenyl-3H-imidazo[4,5-b]pyridin-3-yl)benzyl)carbamoyl)-2-fluorobenzenesulfonic acid 9 (64.7 mg, 6.1% yield) as a yellow solid. ¹H NMR (400 MHz, DMSO-d₆) δ_{H}=9.03 (s, 1H), 8.35 (d, J=8.4 Hz, 2H), 8.10 (d, J=6.0 Hz, 1H), 8.06 (d, J=8.0 Hz, 3H), 7.97 (d, J=7.2 Hz, 1H), 7.78 (d, J=6.8 Hz, 1H), 7.61-7.54 (m, 3H), 7.54-7.46 (m, 4H), 7.45-7.37 (m, 1H), 7.20 (d, J=7.6 Hz, 1H), 6.96 (d, J=6.4 Hz, 1H), 4.55 (d, J=5.6 Hz, 2H). HPLC Rₜ=3.630 min in 8 min chromatography, purity 96.3%. LCMS Rₜ=2.364 min in 4 min chromatography, purity 97.9%, MS ESI calcd. for 594.15 [M+H]⁺ 595.15, found 595.1.

### Example 10. Synthesis of N-(4-(2-(2-aminopyridin-3-yl)-5-phenyl-3H-imidazo[4,5-b]pyridin-3-yl)benzyl)-3-sulfamoylbenzamide (Compound 10)

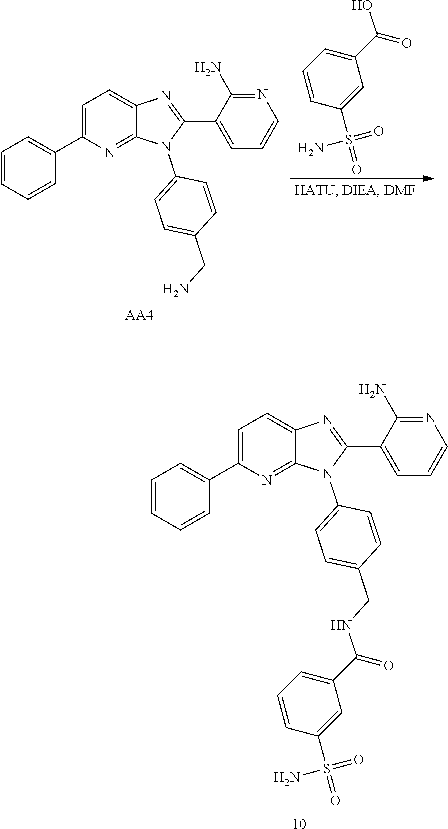

Step 1. To a solution of 3-sulfamoylbenzoic acid (51.0 mg, 0.255 mmol) in DMF (3.00 mL), 3-(3-(4-(aminomethyl)phenyl)-5-phenyl-3H-imidazo[4,5-b]pyridin-2-yl)pyridin-2-amine AA4 (100 mg, 0.255 mmol) and DIEA (99.0 mg, 0.764 mmol) were added. HATU (116 mg. 0.306 mmol) was then added, and the mixture allowed to stir at 25° C. for 2 hrs. The reaction mixture was poured into water (30 mL), and the resulting mixture was extracted with DCM (20 mL×3). The combined organic phase was washed with water (30 mL), dried over anhydrous Na₂SO₄, filtered and concentrated. The crude product was purified by prep-HPLC (column: welch xtimate c18 150*40 mm*10 μm; mobile phase: [water (NH₄HCO₃)-ACN]; B %: 22%-62%: 32 min) to give an impure product. The impure product was purified by prep-HPLC (welch ultimate xb-nh2 250*50*10 μm; mobile phase: [Heptane-EtOH (0.1% NH₃H₂O)]; B %: 15%-45%: 15 min) to give an impure product. The impure product was purified by prep-HPLC (column: welch xtimate c18 150*40 mm*10 μm; mobile phase: [water (HCl)-ACN]; B %: 10%-50%: 36 min) to give N-(4-(2-(2-aminopyridin-3-yl)-5-phenyl-3H-imidazo[4,5-b]pyridin-3-yl)benzyl)-3-sulfamoylbenzamide 10 (9.1 mg, 6.1% yield) as a light yellow solid. ¹H NMR (400 MHz, DMSO-d₆) δ_{H}=9.45-9.39 (m, 1H), 8.42-8.37 (m, 1H), 8.34 (d, J=8.0 Hz, 1H), 8.17-8.12 (m, 1H), 8.10-8.02 (m, 4H), 8.01-7.97 (m, 1H), 7.88-7.76 (m, 1H), 7.71 (t, J=7.6 Hz, 1H), 7.58-7.44 (m, 8H), 7.43-7.38 (m, 1H), 6.91-6.74 (m, 1H), 4.63 (d, J=5.6 Hz, 2H). HPLC Rₜ=2.869 min in 8 min chromatography, purity 99.4%. LCMS Rₜ=2.677 min in 4 min: Agilent PoroShell 120 EC-C18 2.7 μm 3.0*50 mm; purity 98.4%, MS ESI calcd, for 575.17, [M+H]⁺ 576.17, found 576.2.

### Example 11. Synthesis of N-(4-(2-(2-aminopyridin-3-yl)-5-phenyl-3H-imidazo[4,5-b]pyridin-3-yl)benzyl)-4-sulfamoylbenzamide (Compound 11)

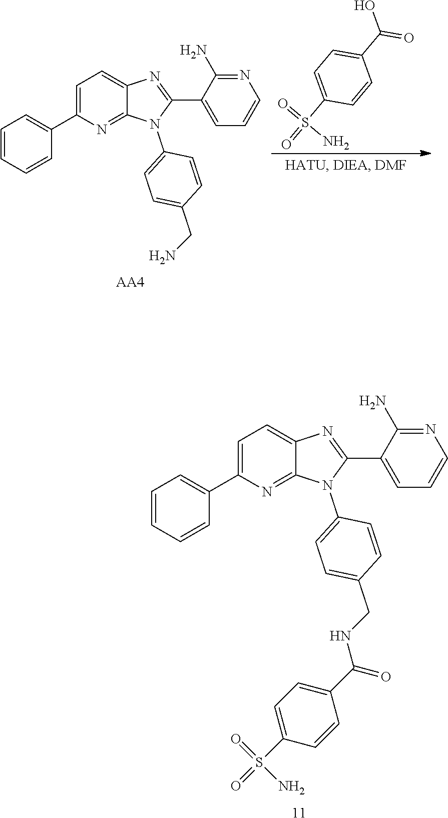

Step 1. To a solution of 3-(3-(4-(aminomethyl)phenyl)-5-phenyl-3H-imidazo[4,5-b]pyridin-2-yl)pyridin-2-amine AA4 (100 mg, 0.255 mmol) in DMF (3.00 mL), 4-sulfamoylbenzoic acid (51.0 mg, 0.255 mmol) and DIEA (99.0 mg, 0.764 mmol) were added. HATU (116 mg, 0.306 mmol) was then added, and the mixture stirred at 25° C. for 2 hrs. The reaction mixture was poured into water (30 mL), and the resulting mixture was extracted with DCM (20 mL×3). The combined organic phase was washed with water (30 mL), dried over anhydrous Na₂SO₄, filtered and concentrated. The crude product was purified by prep-HPLC (column: welch xtimate c18 150*40 mm*10 μm; mobile phase: [water (NH₃H₂O+NH₄HCO₃)-ACN]; B %: 22%-62%: 32 min) to give an impure product. The impure product was purified by prep-HPLC (Welch Ultimate XB—NH₂ 250*50*10 μm; mobile phase: [Heptane-EtOH (0.1% NH₃H₂O)]; B %: 15%-45%: 15 min) to give an impure product. The impure product was purified by prep-HPLC (column: welch xtimate c18 150*40 mm*10 μm; mobile phase: [water (HCl)-ACN]; B %: 10%-50%: 36 min) to give N-(4-(2-(2-aminopyridin-3-yl)-5-phenyl-3H-imidazo[4,5-b]pyridin-3-yl)benzyl)-4-sulfamoylbenzamide 11 (8.4 mg, 5.72% yield) as a light yellow solid. ¹H NMR (400 MHz, DMSO-d₆) δ_{H}=9.36 (t, J=5.6 Hz, 1H), 8.45-8.27 (m, 2H), 8.12-8.02 (m, 6H), 7.95-7.90 (m, 2H), 7.90-7.84 (m, 1H), 7.59-7.45 (m, 8H), 7.44-7.38 (m, 1H), 6.88 (t, J=6.8 Hz, 1H), 4.62 (d, J=6.0 Hz, 2H). HPLC Rₜ=2.733 min in 8 min chromatography, purity 99.9%. LCMS Rₜ=2.647 min in 4 min; Agilent PoroShell 120 EC-C18 2.7 μm 3.0*50 mm; purity 100%, MS ESI calcd, for $75.17, [M+H]⁺ 576.17, found 576.2.

### Example 12. Synthesis of N-[[4-[2-(2-amino-3-pyridyl)-5-phenyl-imidazo[4,5-b]pyridin-3-yl]phenyl methyl]-3-ethylsulfonyl-benzamide (Compound 12)

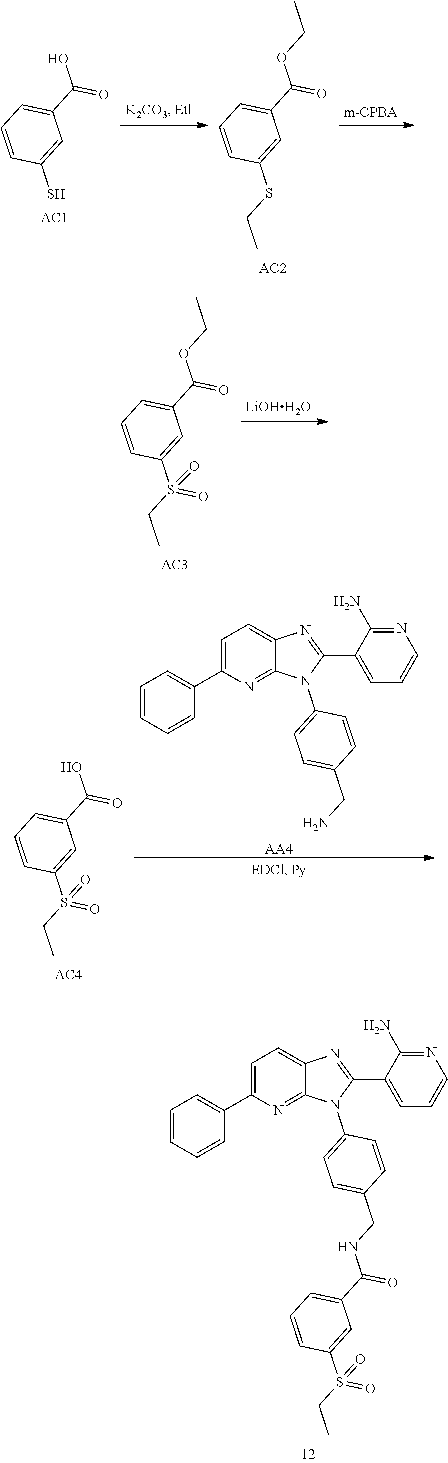

Step 1. To a stirred solution of 3-sulfanylbenzoic acid AC1 (500 mg, 3.20 mmol) and K₂CO₃ (1.79 g. 13.0 mmol) in DMF (20.0 mL), iodoethane (5.00 g, 32.4 mmol) was added and the reaction mixture stirred at 25° C. under N₂ for 16 hrs. The reaction mixture was diluted with EtOAc (20 mL), washed with water (50 mL) and brine (50 mL×3), dried over anhydrous sodium sulfate, filtered and the filtrate was concentrated to afford ethyl 3-ethylsulfanylbenzoate AC2 (600 mg, 87.9% yield) as a yellow oil. ¹H NMR (400 MHz, CDCl₃) δ_{H}=7.98 (t, J=1.6 Hz, 1H), 7.83 (td, J=1.2, 7.6 Hz, 1H), 7.53-7.46 (m, 1H), 7.40-7.31 (m, 1H), 4.38 (q, J=7.2 Hz, 2H), 2.99 (q, J=7.2 Hz, 2H), 1.39 (1, J=7.2 Hz, 3H), 1.33 (t, J=7.2 Hz, 3H).

Step 2. To a stirred solution of ethyl 3-ethylsulfanylbenzoate AC2 (150 mg, 0.710 mmol) in DCM (2.00 mL), m-CPBA (308 mg, 1.78 mmol, 85%) was added in portions at 0° C., and the reaction mixture stirred at 0° C., under N₂, for 2 hrs. The reaction mixture was quenched with sat. aqueous Na₂SO₃ solution (50 mL) and subsequently extracted with EtOAc (50 mL×2). The combined organic layers were washed with Na₂SO₃ (50 mL), brine (50 mL), dried over anhydrous sodium sulfate, filtered and the filtrate was concentrated to afford ethyl 3-ethylsulfonylbenzoate AC3 (100 mg, 57.8% yield) as a yellow oil. ¹H NMR (400 MHz, CDCl₃) δ=8.56 (t, J=1.6 Hz, 1H), 8.33 (d, J=7.6 Hz, 1H), 8.10 (d, J=8.0 Hz, 1H), 7.67 (t, J=8.0 Hz, 1H), 4.43 (q, J=7.2 Hz, 2H), 3.16 (q, J=7.6 Hz, 2H), 1.42 (t, J=7.2 Hz, 3H), 1.33-1.28 (m, 3H).

Step 3. To a stirred solution of ethyl 3-ethylsulfonylbenzoate AC3 (100 mg, 0.410 mmol) in THF (1.00 mL) and water (1.00 mL), LiOH·H₂O (33.0 mg, 0.830 mmol) was added and the reaction mixture stirred at 25° C. under N₂ for 16 hrs. The reaction mixture was adjusted to a pH ~5 with 1 M HCl and extracted with EtOAc (10 mL×2). The combined organic layers were washed with brine (10 mL×2), dried over anhydrous sodium sulfate, filtered and the filtrate was concentrated to afford 3-ethylsulfonylbenzoic acid AC4 (80.0 mg, 90.5% yield) as a white solid. ¹H NMR (400 MHz, DMSO-d₆) δ=13.57 (s, 1H), 8.35 (t, J=1.6 Hz, 1H), 8.31-8.24 (m, 1H), 8.17-8.11 (m, 1H), 7.81 (t, J=8.0 Hz, 1H), 3.40-3.35 (m, 2H), 1.10 (t, J=7.6 Hz, 3H).

Step 4. To a stirred solution of 3-ethylsulfonylbenzoic acid AC4 (55.0 mg, 0.260 mmol) and 3-(3-(4-(aminomethyl)phenyl)-5-phenyl-3H-imidazo[4,5-b]pyridin-2-yl)pyridin-2-amine AA4 (100 mg, 0.260 mmol) in pyridine (2.00 mL), EDCI (54.0 mg, 0.280 mmol) was added and the reaction mixture stirred at 25° C., under N₂, for 16 hrs. The reaction mixture was adjusted to pH ~5 with 1 M HCl and concentrated to afford a residue. The crude product was purified by prep-HPLC (column Welch Xtimate C18 150×30 mm×5 μm, condition water (FA)-ACN, begin B: 23, end B: 53) to afford N-[[4-[2-(2-amino-3-pyridyl)-5-phenyl-imidazo[4,5-b]pyridin-3-yl]phenyl]methyl]-3-ethylsulfonyl-benzamide 12 (29.0 mg, 19.2%) yield) as a white solid. ¹H NMR (400 MHz, DMSO-d₆) δ_{H}=9.46 (t, J=6.0 Hz, 1H), 8.42 (s, 1H), 8.33-8.21 (m, 2H), 8.11-7.92 (m, 5H), 7.80 (t, J=8.0 Hz, 1H), 7.56-7.34 (m, 7H), 7.22 (dd, J=1.6, 7.6 Hz, 1H), 6.94 (s, 2H), 6.42 (dd, J=4.8, 7.6 Hz, 1H), 4.64 (d, J=6.0 Hz, 2H), 3.35 (q, J=7.2 Hz, 2H), 1.12 (t, J=7.2 Hz, 3H). HPLC Rₜ=5.220 min in 8 min chromatography, purity 98.8%. LCMS Rₜ=2.073 min in 4 min chromatography, purity 98.9%, MS ESI calcd, for 588.19 [M+H]⁺ 589.19, found 589.0.

### Example 13. Synthesis of N-[[4-[2-(2-amino-3-pyridyl)-5-phenyl-imidazo[4,5-b]pyridin-3-yl]phenyl]methyl]-6-ethylsulfonyl-pyridine-2-carboxamide (Compound 13)

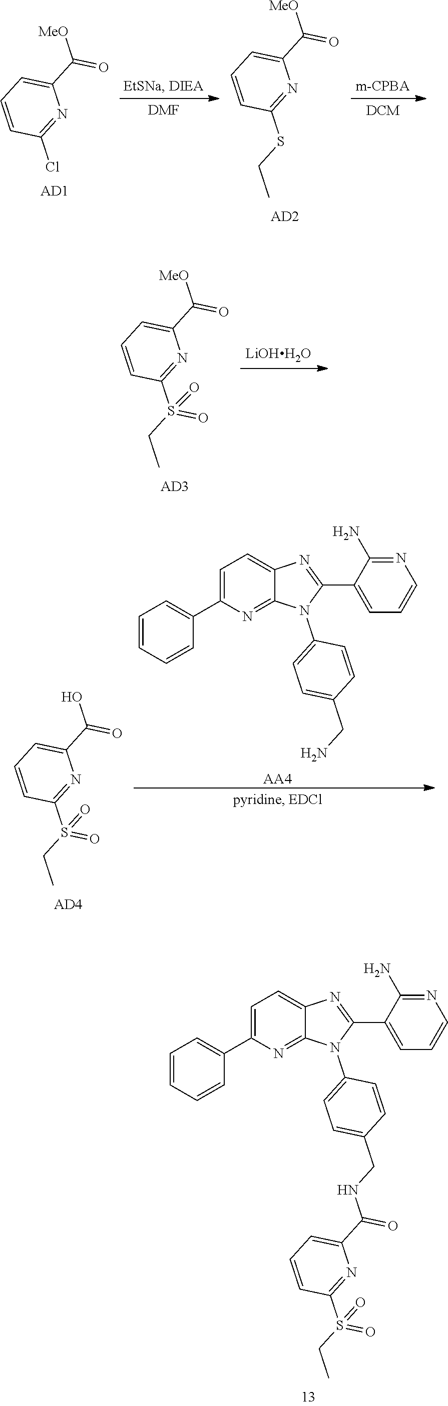

Step 1. To a stirred solution of methyl 6-chloropyridine-2-carboxylate AD1 (1.00 g, 5.83 mmol) and sodium ethanethiolate (600 mg, 7.14 mmol) in DMF (20.0 mL), DIEA (2.10 mL, 11.7 mmol) was added, and the reaction stirred at 50° C. under N₂ for 16 hrs. The reaction was diluted with EtOAc (50 mL), washed with water (100 mL), brine (100 mL×2), dried over anhydrous sodium sulfate, filtered and the filtrate was concentrated and purified by flash chromatography on silica gel (20% of EtOAc in PE) to afford methyl 6-ethylsulfanylpyridine-2-carboxylate AD2 (60.0 mg, 5.20% yield) as a colorless oil. ¹H NMR (400 MHz, CDCl₃) δ=7.77 (d, J=7.6 Hz, 1H), 7.59 (t, J=7.6 Hz, 1H), 7.31 (d, J=8.0 Hz, 1H), 3.97 (s, 3H), 3.24 (q, J=7.2 Hz, 2H), 1.40 (t, J=7.2 Hz, 3H).

Step 2. To a stirred solution of methyl 6-ethylsulfanylpyridine-2-carboxylate AD2 (60.0 mg, 0.300 mmol) in DCM (2.00 mL), m-CPBA (154 mg, 0.760 mmol, 85%) was added at 0° C. and the reaction mixture stirred at 0° C. under N₂ for 1 hr. The reaction mixture was quenched with Na₂SO₃ (30 mL), extracted with EtOAc (20 mL×2), dried over anhydrous sodium sulfate, filtered and the filtrate was concentrated to afford methyl 6-ethylsulfonylpyridine-2-carboxylate AD3 (100 mg, crude) as a yellow oil. ¹H NMR (400 MHz, CDCl₃) δ=8.33 (dd, J=1.2, 7.6 Hz, 1H), 8.27 (dd, J=1.2, 7.6 Hz, 1H), 8.16-8.09 (m, 1H), 4.02 (s, 3H), 3.53 (q, J=7.6 Hz, 2H), 1.35 (I, J=7.6 Hz, 3H).

Step 3. To a stirred solution of methyl 6-ethylsulfonylpyridine-2-carboxylate AD3 (100 mg, 0.440 mmol) in THF (1.00 mL) and water (1.00 mL), LiOH·H₂O) (35.0 mg, 0.870 mmol) was added and the reaction mixture stirred at 25° C. under N₂ for 16 hrs. The reaction mixture was concentrated to afford lithium 6-(ethylsulfonyl) picolinate AD4 (90.0 mg, crude) as a yellow solid. ¹H NMR (400 MHz, DMSO-d₆) δ_{H}=8.16-8.04 (m, 2H), 8.01-7.89 (m, 1H), 3.49-3.42 (m, 2H), 1.11 (t, J=7.6 Hz, 3H).

Step 4. To a stirred solution of 3-(3-(4-(aminomethyl)phenyl)-5-phenyl-3H-imidazo[4,5-b]pyridin-2-yl)pyridin-2-amine AA4 (100 mg, 0.250 mmol) and lithium 6-(ethylsulfonyl) picolinate AD4 (55.0 mg, 0.250 mmol) in pyridine (2.00 mL), EDCI (54.0 mg, 0.280 mmol) was added and the reaction mixture stirred at 25° C. under N₂ for 16 hrs. The reaction mixture was quenched with water (10 mL) and extracted with EtOAc (10 mL×2). The combined organic layers were washed with 1 M HCl solution (15 mL×2), brine (20 mL), dried over anhydrous sodium sulfate, filtered and the filtrate was concentrated to afford a residue. The crude product was purified by prep-HPLC (column Welch Xtimate C18 150×30 mm×5 μm, condition: water (NH₃H₂O+NH₄HCO₃)-ACN, begin B: 44, end B: 74) to afford N-[[4-[2-(2-amino-3-pyridyl)-5-phenyl-imidazo[4,5-b]pyridin-3-yl]phenyl]methyl]-6-ethylsulfonyl-pyridine-2-carboxamide 13 (3.70 mg, 2.50% yield) as a yellow solid. ¹H NMR (400 MHz, DMSO-d₆) δ=9.64 (t, J=6.4 Hz, 1H), 8.44-8.31 (m, 2H), 8.29-8.16 (m, 2H), 8.08-7.89 (m, 4H), 7.56-7.35 (m, 7H), 7.23 (dd, J=1.6, 7.6 Hz, 1H), 6.92 (s, 2H), 6.41 (dd, J=4.8, 7.6 Hz, 1H), 4.67 (d, J=6.4 Hz, 2H), 3.77 (q, J=7.2 Hz, 2H), 1.16 (t, J=7.2 Hz, 3H). HPLC Rₜ=5.239 min in 8 min chromatography, purity 96.6%. LCMS Rₜ=2.041 min in 4 min chromatography, purity 98.1%, MS ESI calcd, for 589.19 [M+H]⁺ 590.19, found 590.1.

### Example 14. Synthesis of 4-((4-(2-(2-aminopyridin-3-yl)-5-phenyl-3H-imidazo[4,5-b]pyridin-3-yl)benzyl)carbamoyl)benzoic acid (Compound 14)

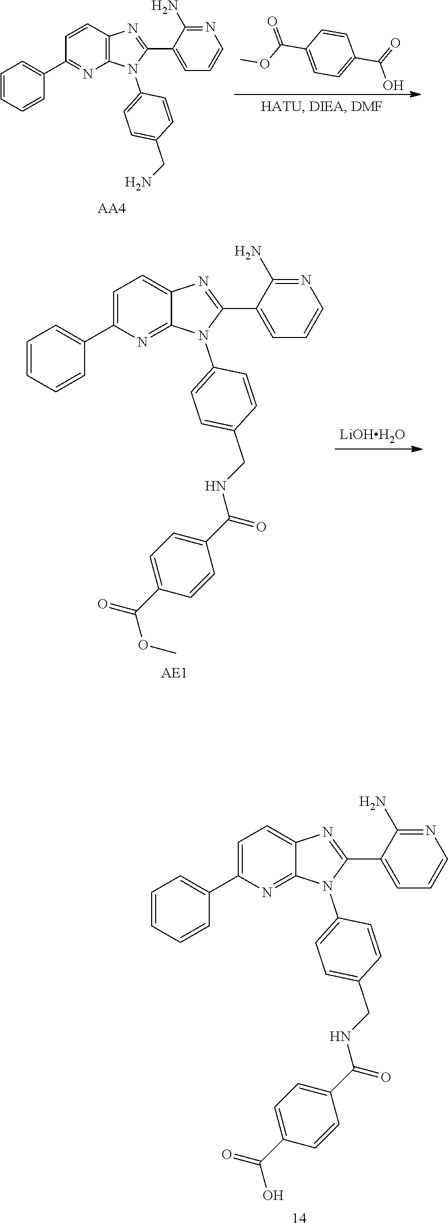

Step 1. To a solution of 3-(3-(4-(aminomethyl)phenyl)-5-phenyl-3H-imidazo[4,5-b]pyridin-2-yl)pyridin-2-amine AA4 (200 mg, 0.510 mmol) and 4-methoxycarbonylbenzoic acid (92.0 mg, 0.510 mmol) in DMF (5.00 mL), DIEA (198 mg, 1.53 mmol) and HATU (291 mg, 0.764 mmol) were added and the mixture stirred at 25° C. for 2 hrs. Water (2 mL) was added to the reaction mixture and the resulting mixture was extracted with EtOAc (5 ml×3). The combined organic phase was washed with brine (10 mL), dried over anhydrous Na₂SO₄, filtered and concentrated to dryness. The residue was purified by flash silica gel chromatography (eluent with 0-80% of EtOAc in PE) to afford methyl 4-((4-(2-(2-aminopyridin-3-yl)-5-phenyl-3H-imidazo[4,5-b]pyridin-3-yl)benzyl)carbamoyl)benzoate AE1 (275 mg, 0.496 mmol) as a light yellow solid. ¹H NMR (DMSO-d₆, 400 MHz) δ_{H}=9.35 (t, J=6.0 Hz, 1H), 8.26 (d, J=8.4 Hz, 1H), 8.07-7.96 (m, 8H), 7.52-7.44 (m, 6H), 7.42-7.35 (m, 1H), 7.26-7.18 (m, 1H), 6.95 (s, 2H), 6.42 (dd, J=5.2, 8.0 Hz, 1H), 4.63 (d, J=6.0 Hz, 2H), 3.89 (s, 3H).

Step 2. To a solution of methyl 4-((4-(2-(2-aminopyridin-3-yl)-5-phenyl-3H-imidazo[4,5-b]pyridin-3-yl)benzyl)carbamoyl)benzoate AE1 (150 mg, 0.270 mmol) in THF (4.00 mL) and water (4.00 mL), LiOH·H₂O (17.0 mg, 0.406 mmol) was added. The mixture was stirred at 25° C. for 4 hrs. The reaction mixture was subsequently concentrated to dryness, and the residue was purified by prep-HPLC (Column: Welch Xtimate C18 150*30 mm*5 μm, mobile phase: [water (NH₃H₂O+NH₄HCO₃)-ACN]; B %: 26%-66%; 20 min) to afford 4-((4-(2-(2-aminopyridin-3-yl)-5-phenyl-3H-imidazo[4,5-b]pyridin-3-yl)benzyl)carbamoyl)benzoic acid 14 (46.4 mg, 0.0858 mmol, 31.7% yield) as a light yellow solid. ¹H NMR (DMSO-d₆, 400 MHz) δ_{H}=9.29 (t, J=6.0 Hz, 1H), 8.26 (d, J=8.4 Hz, 1H), 8.04-7.97 (m, 8H), 7.51-7.44 (m, 6H), 7.41-7.36 (m, 1H), 7.24-7.19 (m, 1H), 6.94 (s, 2H), 6.42 (dd, J=4.8, 7.6 Hz, 1H), 4.62 (d, J=6.0 Hz, 2H). HPLC R: =2.747 min in 8 min chromatography, purity 99.9%. LCMS Rₜ=1.201 min in 2 min chromatography, purity 99.8%, MS ESI calcd, for $40.19, [M+H]+ 541.19, found 541.0.

### Example 15, Synthesis of 4-((4-(2-(2-aminopyridin-3-yl)-5-phenyl-3H-imidazo[4,5-b]pyridin-3-yl)benzyl)carbamoyl)-3-methylbenzoic acid (Compound 15)

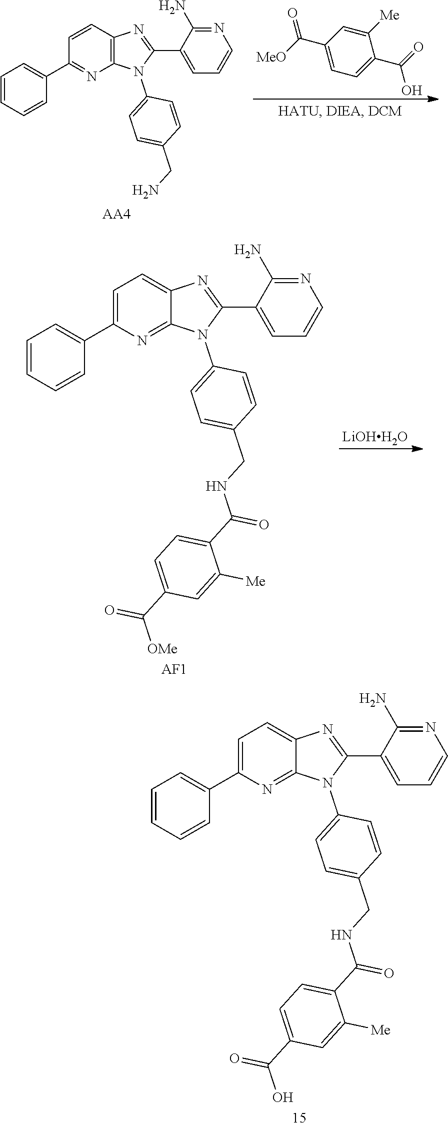

Step 1. To a solution of 3-(3-(4-(aminomethyl)phenyl)-5-phenyl-3H-imidazo[4,5-b]pyridin-2-yl)pyridin-2-amine AA4 (100 mg, 0.255 mmol) in DCM (5.00 mL), 4-(methoxycarbonyl)-2-methylbenzoic acid (74.0 mg, 0.382 mmol), DIEA (0.140 mL, 0.764 mmol) and HATU (145 mg, 0.382 mmol) were added at 25° C., and the mixture was stirred for 16 hrs. The reaction mixture was concentrated directly and the resulting residue was purified by flash silica gel chromatography (eluent with 0-3% of MeOH/DCM) to give methyl 4-((4-(2-(2-aminopyridin-3-yl)-5-phenyl-3H-imidazo[4,5-b]pyridin-3-yl)benzyl)carbamoyl)-3-methylbenzoate AF1 (89.0 mg, 0.157 mmol, 61.4% yield) as a yellow solid. ¹H NMR (400 MHz, DMSO-d₆) δ=9.08 (t, J=6.0 Hz, 1H), 8.27 (d, J=8.4 Hz, 1H), 8.06-7.97 (m, 4H), 7.89-7.79 (m, 2H), 7.57-7.43 (m, 7H), 7.43-7.36 (m, 1H), 7.21 (dd, J=7.6, 1.6 Hz, 1H), 6.98 (s, 2H), 6.41 (dd, J=7.6, 4.8 Hz, 1H), 4.57 (d, J=6.0 Hz, 2H), 3.87 (s, 3H), 2.39 (s, 3H).

Step 2. To a solution of methyl 4-((4-(2-(2-aminopyridin-3-yl)-5-phenyl-3H-imidazo[4,5-b]pyridin-3-yl)benzyl)carbamoyl)-3-methylbenzoate AF1 (89.0 mg, 0.157 mmol) in THF (2.00 mL) and water (2.00 mL), LiOH·H₂O (13.0 mg, 0.314 mmol) was added at 25° C., and the mixture stirred for 2 hrs. The reaction mixture was concentrated to remove THE, and the residue was purified by prep-HPLC (Column: Xtimate C18 150*40 mm*10 μm; condition: water (HCl)-ACN; begin B: 8-38%) to give 4-((4-(2-(2-aminopyridin-3-yl)-5-phenyl-3H-imidazo[4,5-b]pyridin-3-yl)benzyl)carbamoyl)-3-methylbenzoic acid 15 (25.0 mg, 0.0446 mmol, 25.4% yield) as a yellow solid. ¹H NMR (400 MHz, DMSO-d₆) δ=9.08 (t, J=6.0 Hz, 1H), 8.71-8.27 (m, 3H), 8.14 (dd, J=6.4, 1.6 Hz, 1H), 8.10-8.02 (m, 3H), 7.90 (dd, J=7.6, 1.6 Hz, 1H), 7.86-7.78 (m, 2H), 7.60-7.45 (m, 7H), 7.45-7.39 (m, 1H), 6.89 (dd, J=7.6, 6.4 Hz, 1H), 4.56 (d, J=6.0 Hz, 2H), 2.37 (s, 3H). HPLC Rₜ=3.680 min in 8 min chromatography, purity 99.7%. LCMS Rₜ=1.190 min in 2 min chromatography, purity 97.6%, MS ESI calcd, for 554.21 [M+H]⁺ 555.21, found 555.3.

### Example 16, Synthesis of 4-((4-(2-(2-aminopyridin-3-yl)-5-phenyl-3H-imidazo[4,5-b]pyridin-3-yl)benzyl)carbamoyl)-3-fluorobenzoic acid (Compound 16)

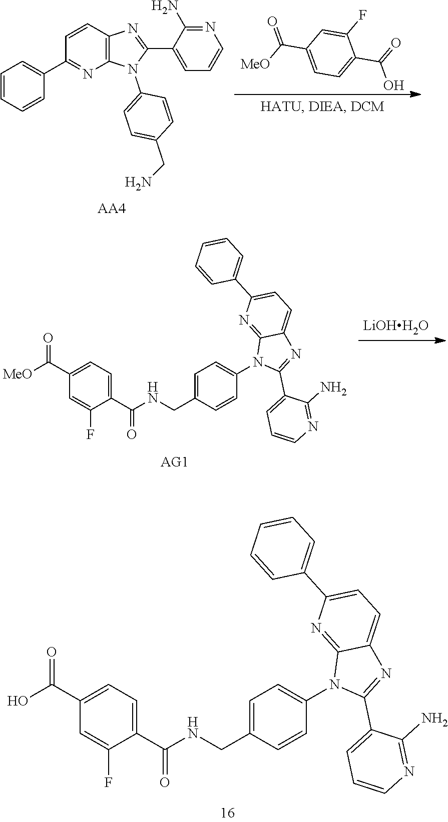

Step 1. To a solution of 3-(3-(4-(aminomethyl)phenyl)-5-phenyl-3H-imidazo[4,5-b]pyridin-2-yl)pyridin-2-amine AA4 (100 mg, 0.250 mmol) in DCM (3.00 mL), 2-fluoro-4-methoxycarbonyl-benzoic acid (50.0 mg, 0.250 mmol) and DIEA (99.0 mg, 0.760 mmol) were added. HATU (116 mg, 0.300 mmol) was then added, and the mixture stirred at 25° C. for 2 hrs. The reaction mixture was poured into water (30 mL), and the resulting mixture was extracted with DCM (20 mL×3). The combined organic phase was washed with water (30 mL), dried over anhydrous Na₂SO₄ and concentrated to dryness. The crude product was purified by combi-flash column (EtOAc in PE=78%) to give methyl 4-((4-(2-(2-aminopyridin-3-yl)-5-phenyl-3H-imidazo[4,5-b]pyridin-3-yl)benzyl)carbamoyl)-3-fluorobenzoate AG1 (140 mg, 94.6% yield) as a yellow solid. ¹H NMR (400 MHz, DMSO-d₆) δ_{H}=9.21 (t, J=5.2 Hz, 1H), 8.27 (d, J=8.4 Hz, 1H), 8.23-8.10 (m, 1H), 8.06-7.95 (m, 4H), 7.89-7.77 (m, 3H), 7.51-7.44 (m, 5H), 7.42-7.35 (m, 1H), 7.22 (dd, J=1.6, 7.6 Hz, 1H), 6.96 (s, 2H), 6.42 (dd, J=5.2, 8.0 Hz, 1H), 4.61 (d, J=6.0 Hz, 2H), 3.89 (s, 3H).

Step 2. To a solution of methyl 4-((4-(2-(2-aminopyridin-3-yl)-5-phenyl-3H-imidazo[4,5-b]pyridin-3-yl)benzyl)carbamoyl)-3-fluorobenzoate AG1 (140 mg, 0.240 mmol) in THF (4.00 mL) and water (2.00 mL), LiOH H₂O (69.0 mg, 1.64 mmol) was added, and the mixture was stirred at 25° C. for 2 hrs. The reaction mixture was diluted with water (2 mL) and purified by prep-HPLC (column: welch xtimate c18 150*40 mm*10 μm; mobile phase: [water (HCl)-ACN], B %: 10%-50%: 36 min) to give 4-((4-(2-(2-aminopyridin-3-yl)-5-phenyl-3H-imidazo[4,5-b]pyridin-3-yl)benzyl)carbamoyl)-3-fluorobenzoic acid 16 (45.1 mg, 33.0% yield) as a yellow solid. ¹H NMR (400 MHz, DMSO-d₆) δ_{H}=9.18 (t, J=6.0 Hz, 1H), 8.58-8.27 (m, 2H), 8.12 (dd, J=1.2, 6.0 Hz, 1H), 8.09-7.98 (m, 3H), 7.90 (dd, J=1.2, 7.6 Hz, 1H), 7.87-7.68 (m, 3H), 7.60-7.46 (m, 5H), 7.45-7.38 (m, 1H), 6.94-6.84 (m, 1H), 4.61 (d, J=6.0 Hz, 2H). HPLC Rₜ=2.430 min in 8 min chromatography, purity 100%. LCMS Rₜ=2.925 min in 4 min; Agilent PoroShell 120 EC-C18 2.7 μm 3.0*50 mm; purity 91.2%, MS ESI calcd, for 558.18, [M+H]⁺ 559.18, found 559.2.

### Example 17. Synthesis of 3-((4-(2-(2-aminopyridin-3-yl)-5-phenyl-3H-imidazo[4,5-b]pyridin-3-yl)benzyl)carbamoyl)benzoic acid (Compound 17)

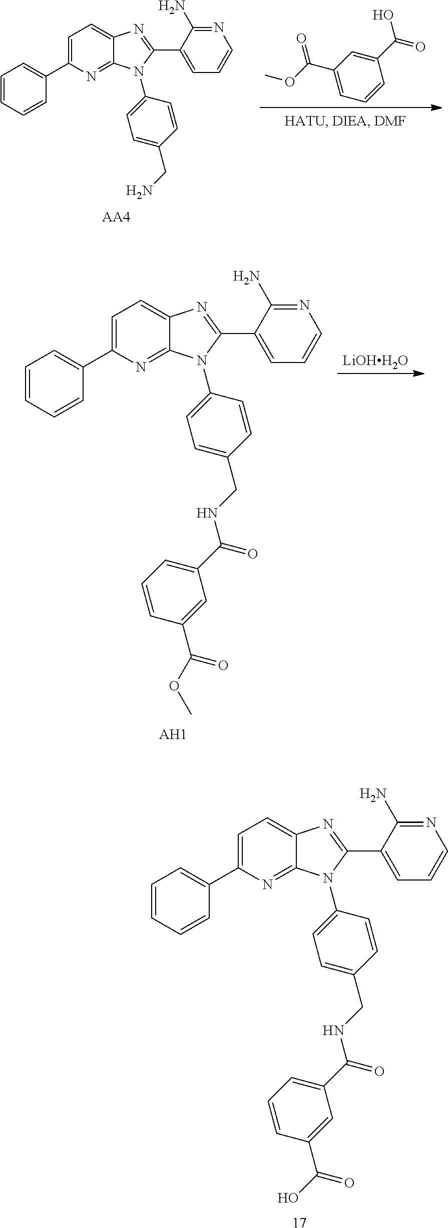

Step 1. To a solution of 3-(3-(4-(aminomethyl)phenyl)-5-phenyl-3H-imidazo[4,5-b]pyridin-2-yl)pyridin-2-amine AA4 (220 mg, 0.561 mmol) and 3-methoxycarbonylbenzoic acid (101 mg, 0.561 mmol) in DMF (5.00 mL), DIEA (217 mg, 1.68 mmol) and HATU (320 mg, 0.841 mmol) were added and the mixture stirred at 25° C. for 2 hrs. Water (2 mL) was added and the resulting mixture was extracted with EtOAc (5 mL×3). The combined organic phase was washed with brine (10 mL), dried over anhydrous Na₂SO₄, filtered and concentrated. The residue was purified by flash silica gel chromatography (eluent of 0-80% EtOAc in PE) to afford methyl 3-((4-(2-(2-aminopyridin-3-yl)-5-phenyl-3H-imidazo[4,5-b]pyridin-3-yl)benzyl)carbamoyl)benzoate AH1 (280 mg, 0.505 mmol) as a light yellow solid. ¹H NMR (DMSO-d₆, 400 MHz) δ_{H}=9.39 (t, J=6.0 Hz, 1H), 8.54 (s, 1H), 8.26 (d, J=8.4 Hz, 1H), 8.23-8.11 (m, 3H), 8.05-7.96 (m, 4H), 7.52-7.43 (m, 6H), 7.41-7.36 (m, 1H), 7.25-7.19 (m, 1H), 6.95 (s, 2H), 6.42 (dd, J=4.8, 7.6 Hz, 1H), 4.63 (d, J=6.0 Hz, 2H), 3.90 (s, 3H).

Step 2. To a solution of methyl 3-((4-(2-(2-aminopyridin-3-yl)-5-phenyl-3H-imidazo[4,5-b]pyridin-3-yl)benzyl)carbamoyl)benzoate AH1 (140 mg, 0.252 mmol) in THF (4.00 mL) and water (4.00 mL), LiOH·H₂O) (16.0 mg, 0.379 mmol) was added and the mixture stirred at 25° C. for 4 hrs. The reaction mixture was concentrated and the resulting residue was purified by prep-HPLC (Column: Welch Xtimate C18 150*30 mm*5 μm; mobile phase: [water (NH₃H₂O+NH₄HCO₃)-ACN]; B %: 26%-66%; 20 min) to afford 3-((4-(2-(2-aminopyridin-3-yl)-5-phenyl-3H-imidazo[4,5-b]pyridin-3-yl)benzyl)carbamoyl)benzoic acid 17 (30.4 mg, 0.0562 mmol, 22.3% yield) as a light yellow solid. ¹H NMR (DMSO-d₆, 400 MHz) δ_{H}=9.34 (t, J=6.0 Hz, 1H), 8.52 (s, 1H), 8.26 (d, J=8.4 Hz, 1H), 8.16-8.08 (m, 2H), 8.05-7.96 (m, 4H), 7.60 (t, J=7.8 Hz, 1H), 7.51-7.44 (m, 6H), 7.41-7.36 (m, 1H), 7.24-7.19 (m, 1H), 6.94 (s, 2H), 6.42 (dd, J=4.8, 7.6 Hz, 1H), 4.62 (d, J=6.0 Hz, 2H). HPLC Rₜ=2.815 min in 8 min chromatography, purity 100%. LCMS Rₜ=1.216 min in 2 min chromatography, purity 99.9%, MS ESI calcd, for $40.58, [M+H]⁺ 541.58, found 540.9.

### Example 18. Synthesis of 3-((4-(2-(2-aminopyridin-3-yl)-5-phenyl-3H-imidazo[4,5-b]pyridin-3-yl)benzyl)carbamoyl)-2-methylbenzoic acid (Compound 18)

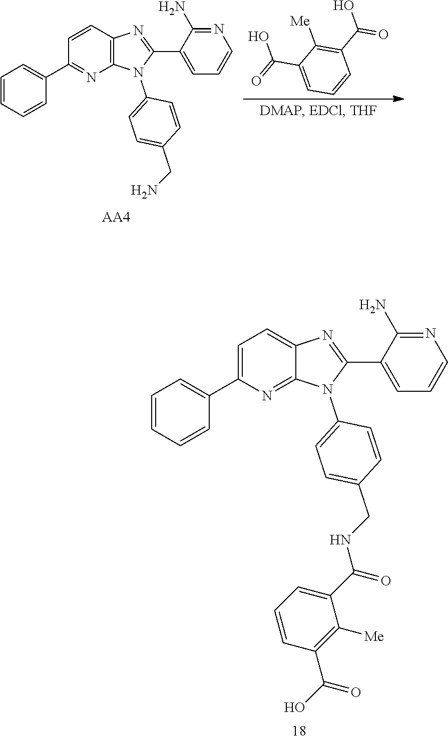

Step 1. To a mixture of 2-methylbenzene-1,3-dicarboxylic acid (92.0 mg, 0.510 mmol) in THF (5.00 mL), 3-(3-(4-(aminomethyl)phenyl)-5-phenyl-3H-imidazo[4,5-b]pyridin-2-yl)pyridin-2-amine AA4 (200 mg, 0.510 mmol) and DMAP (62.0 mg, 0.510 mmol) were added. EDCI (142 mg, 0.917 mmol) was then added, and the mixture was stirred at 25° C. for 12 hrs. The reaction mixture was diluted with water (30 mL), and the resulting mixture was extracted with EtOAc (30 mL×3). The water phase was lyophilized. The crude product was purified by HPLC (column: welch xtimate c18 150*40 mm*10 μm; mobile phase: [water (HCl)-ACN]; B %: 10%-50%: 36 min) to give 3-((4-(2-(2-aminopyridin-3-yl)-5-phenyl-3H-imidazo[4,5-b]pyridin-3-yl)benzyl)carbamoyl)-2-methylbenzoic acid 18 (1.90 mg, 0.67% yield) as a white solid. ¹H NMR (400 MHz, DMSO-d₆) δ_{H}=9.06-8.99 (m, 1H), 8.38-8.34 (m. 1H), 8.13-8.02 (m, 4H), 7.85 (dd, J=1.2, 7.2 Hz, 1H), 7.79 (dd, J=1.2, 8.0 Hz, 1H), 7.59-7.53 (m, 4H), 7.53-7.46 (m, 3H), 7.44-7.39 (m, 1H), 7.37-7.32 (m, 1H), 6.89-6.81 (m, 1H), 4.55 (d, J=6.0 Hz, 2H), 2.45 (s, 3H). HPLC R: =2.923 min in 8 min chromatography, purity 99.0%. LCMS Rₜ=2.561 min in 4 min; Agilent PoroShell 120 EC-C18 2.7 μm 3.0*50 mm; purity 95.7%, MS ESI calcd. For 554.21, [M+H]⁺ 555.21, found 555.2.

### Example 19. Synthesis of 3-((4-(2-(2-aminopyridin-3-yl)-5-phenyl-3H-imidazo[4,5-b]pyridin-3-yl)benzyl)carbamoyl)-2-fluorobenzoic acid (Compound 19)

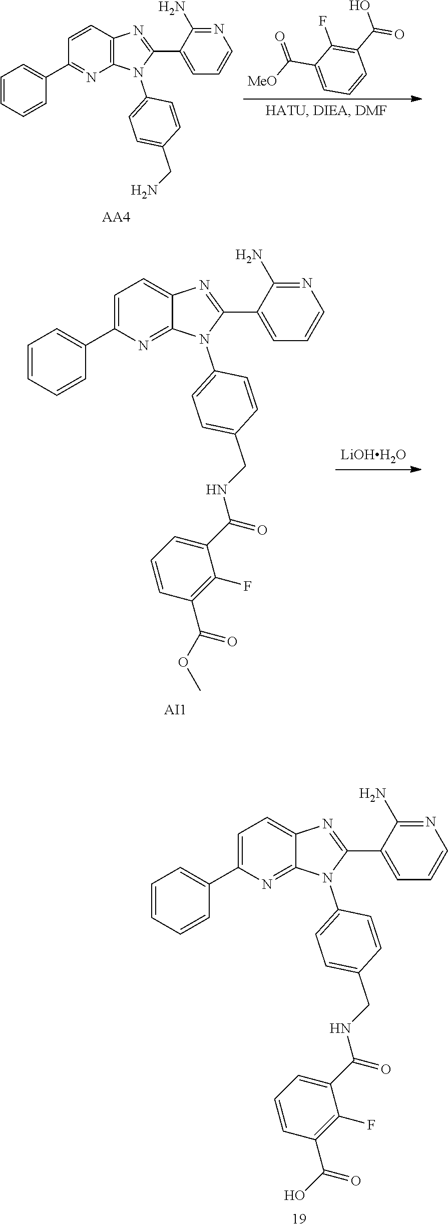

Step 1. To a solution of 3-(3-(4-(aminomethyl)phenyl)-5-phenyl-3H-imidazo[4,5-b]pyridin-2-yl)pyridin-2-amine AA4 (150 mg, 0.382 mmol) and 2-fluoro-3-methoxycarbonyl-benzoic acid (76.0 mg, 0.382 mmol) in DMF (5.00 mL), DIEA (148 mg, 1.15 mmol) and HATU (218 mg, 0.573 mmol) were added and the mixture stirred at 25° C. for 2 hrs. Water (2 mL) was added to the reaction mixture and the resulting mixture was extracted with EtOAc (5 mL×3). The combined organic phase was washed with brine (10 mL), dried over anhydrous Na₂SO₄, filtered and concentrated. The residue was purified by flash silica gel chromatography (eluent with 0-5% of MeOH in DCM) to afford methyl 3-((4-(2-(2-aminopyridin-3-yl)-5-phenyl-3H-imidazo[4,5-b]pyridin-3-yl)benzyl)carbamoyl)-2-fluorobenzoate AI1 (110 mg, 0.192 mmol, 50.3% yield) as a light yellow solid. ¹H NMR (DMSO-d₆, 400 MHz) δ_{H}=9.16 (t, J=6.0 Hz, 1H), 8.27 (d, J=8.4 Hz, 1H), 8.05-7.97 (m, 5H), 7.92-7.87 (m, 1H), 7.54-7.36 (m, 9H), 7.25-7.20 (m, 1H), 6.99-6.95 (m, 1H), 6.43 (dd, J=4.8, 7.6 Hz, 1H), 4.60 (d, J=6.0 Hz, 2H), 3.88 (s, 3H).

Step 2. To a solution of methyl 3-((4-(2-(2-aminopyridin-3-yl)-5-phenyl-3H-imidazo[4,5-b]pyridin-3-yl)benzyl)carbamoyl)-2-fluorobenzoate AI1 (110 mg, 0.192 mmol) in water (3.00 mL) and THF (3.00 mL), LiOH·H₂O (12.0 mg, 0.288 mmol) was added and the mixture stirred at 25° C. for 4 hrs. The reaction mixture was concentrated and the residue was purified by prep-HPLC (Column: Welch Xtimate C18 150*30 mm*5 μm; mobile phase: [water (NH₃H₂O+NH₄HCO₃)-ACN]; B %: 26%-66%; 20 min) to afford 3-((4-(2-(2-aminopyridin-3-yl)-5-phenyl-3H-imidazo[4,5-b]pyridin-3-yl)benzyl)carbamoyl)-2-fluorobenzoic acid 19 (25.9 mg, 0.0460 mmol, 23.9% yield) as a yellow solid. ¹H NMR (DMSO-d₆, 400 MHz) δ_{H}=9.02 (t, J=5.2 Hz, 1H), 8.26 (d, J=8.4 Hz, 1H), 8.07-7.95 (m, 4H), 7.80-7.71 (m, 1H), 7.66-7.60 (m, 1H), 7.52-7.44 (m, 6H), 7.41-7.36 (m, 1H). 7.27-7.19 (m, 2H), 6.96 (s, 2H), 6.47-6.39 (m, 1H), 4.59 (d, J=6.0 Hz, 2H). HPLC Rₜ=2.814 min in 8 min chromatography, purity 99.2%. LCMS Rₜ=1.273 min in 2 min chromatography, purity 98.8%, MS ESI calcd, for 558.18, [M+H]⁺ 559.18, found 558.9.

### Example 20. Synthesis of methyl 3-((4-(2-(2-aminopyridin-3-yl)-5-phenyl-3H-imidazo[4,5-b]pyridin-3-yl)benzyl)carbamoyl)-5-fluorobenzoate (Compound 20)

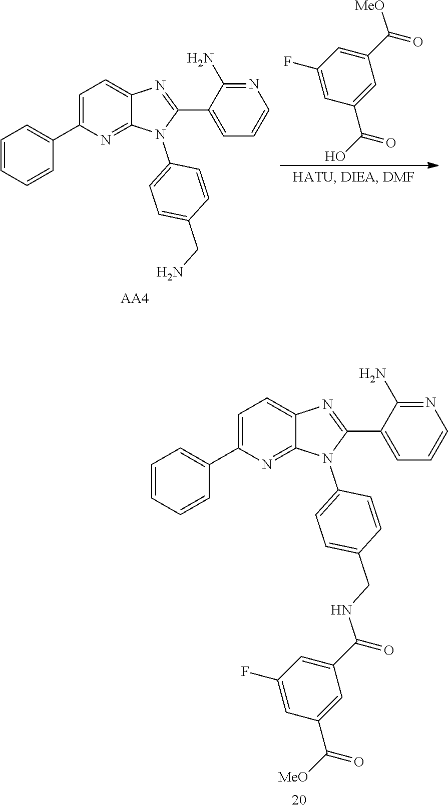

Step 1. To a solution of 3-(3-(4-(aminomethyl)phenyl)-5-phenyl-3H-imidazo[4,5-b]pyridin-2-yl)pyridin-2-amine AA4 (200 mg, 0.510 mmol) and 3-fluoro-5-methoxycarbonyl-benzoic acid (101 mg, 0.510 mmol) in DMF (10.0 mL), DIEA (198 mg, 1.53 mmol) and HATU (233 mg, 0.612 mmol) were added and the mixture stirred at 25° C. for 4 hrs. Water (2 mL) was added to the reaction mixture and the resulting mixture was extracted with EtOAc (5 mL×3). The combined organic phase was washed with brine (10 mL), dried over anhydrous Na₂SO₄, filtered and concentrated. The residue was purified by prep-HPLC (Column: Welch Xtimate C18 150*30 mm*5 μm; mobile phase: [water (NH₄HCO₃)-ACN]; B %: 36%-44%; 36 min) to afford methyl 3-((4-(2-(2-aminopyridin-3-yl)-5-phenyl-3H-imidazo[4,5-b]pyridin-3-yl)benzyl)carbamoyl)-5-fluoro benzoate 20 (24.1 mg, 0.0417 mmol, 8.18% yield) as a light yellow solid. ¹H NMR (400 MHz, DMSO-d₆) δ_{H}=9.46 (t, J=6.0 Hz, 1H), 8.42-8.37 (m, 1H), 8.26 (d, J=8.4 Hz, 1H), 8.06-7.96 (m, 5H), 7.91-7.86 (m, 1H), 7.52-7.43 (m, 6H), 7.41-7.36 (m, 1H), 7.24-7.20 (m, 1H), 6.93 (s, 2H), 6.42 (dd, J=4.8, 7.6 Hz, 1H), 4.63 (d, J=6.0 Hz, 2H), 3.91 (s, 3H). HPLC Rₜ=4.741 min in 8 min chromatography, purity 99.1%. LCMS Rₜ=1.437 min in 2 min chromatography, purity 96.9%, MS ESI calcd, for 572.60, [M+H]⁺ 573.60, found 573.0.

### Example 21. Synthesis of 3-((4-(2-(2-aminopyridin-3-yl)-5-phenyl-3H-imidazo[4,5-b]pyridin-3-yl)benzyl)carbamoyl)-5-fluorobenzoic acid (Compound 21)

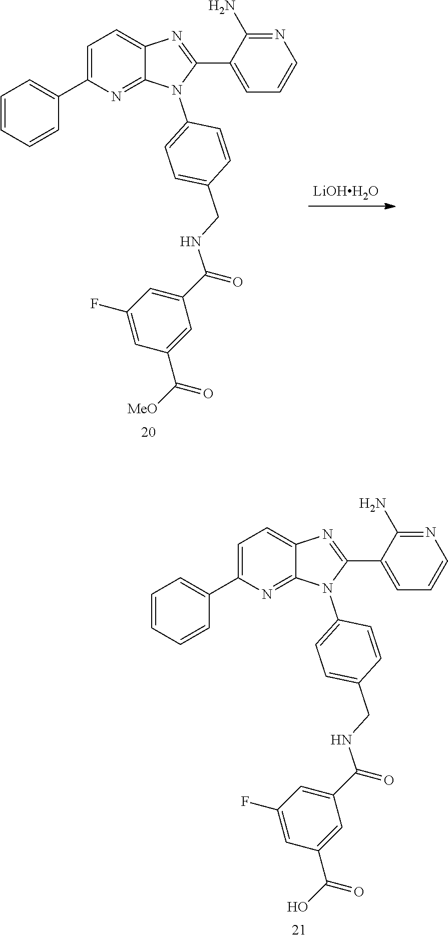

Step 1. To a solution of methyl 3-((4-(2-(2-aminopyridin-3-yl)-5-phenyl-3H-imidazo[4,5-b]pyridin-3-yl)benzyl)carbamoyl)-5-fluorobenzoate 20 (70.0 mg, 0.122 mmol) in THF (3.00 mL) and water (3.00 mL), LiOH·H₂O (26.0 mg, 0.611 mmol) was added and the mixture stirred at 25° C. for 4 hrs. The resulting mixture was concentrated and the residue was purified by prep-HPLC (Column: Welch Xtimate C18 150*30 mm*5 μm, mobile phase: [water (NH₄HCO₃)-ACN]; B %: 36%-44%; 36 min) to afford 3-((4-(2-(2-aminopyridin-3-yl)-5-phenyl-3H-imidazo[4,5-b]pyridin-3-yl)benzyl)carbamoyl)-5-fluorobenzoic acid 21 (56.2 mg, 0.0979 mmol, 80.1% yield) as a light yellow solid. ¹H NMR (400 MHz, DMSO-d₆) δ_{H}=9.44 (t, J=6.0 Hz, 1H), 8.38 (s, 1H), 8.26 (d, J=8.4 Hz, 1H), 8.04-7.94 (m, 5H), 7.86-7.81 (m, 1H), 7.52-7.43 (m, 6H), 7.41-7.36 (m, 1H), 7.24-7.20 (m, 1H), 6.94 (s, 2H), 6.42 (dd, J=4.8, 7.6 Hz, 1H), 4.62 (d, J=6.0 Hz, 2H). HPLC Rₜ=2.158 min in 8 min chromatography, purity 97.3%. LCMS Rₜ=1.271 min in 2 min chromatography, purity 95.9%, MS ESI calcd. for 558.18, [M+H]⁺ 559.18, found 558.9.

### Example 22. Synthesis of methyl 3-((4-(2-(2-aminopyridin-3-yl)-5-phenyl-3H-imidazo[4,5-b]pyridin-3-yl)benzyl)carbamoyl)-4-fluorobenzoate (Compound 22)

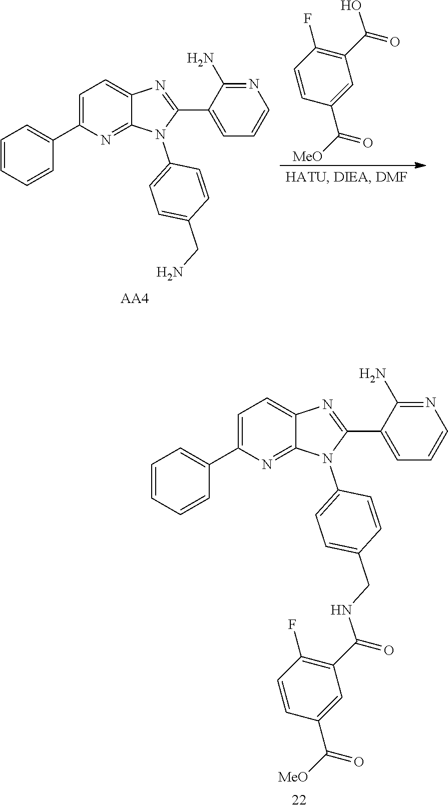

Step 1. To a solution of 3-(3-(4-(aminomethyl)phenyl)-5-phenyl-3H-imidazo[4,5-b]pyridin-2-yl)pyridin-2-amine AA4 (200 mg, 0.510 mmol) and 2-fluoro-5-methoxycarbonyl-benzoic acid (101 mg, 0.510 mmol) in DMF (10.0 mL), DIEA (198 mg, 1.53 mmol) and HATU (233 mg, 0.612 mmol) were added and the mixture stirred at 25° C. for 4 hrs. Water (2 mL) was added to the reaction mixture and the resulting mixture was extracted with EtOAc (5 mL×3). The combined organic phase was washed with brine (10 mL), dried over anhydrous Na₂SO₄, filtered and concentrated. The residue was purified by prep-HPLC (Column: Welch Xtimate C18 150*30 mm*5 μm; mobile phase: [water (NH₄HCO₃)-ACN]; B %: 36%-44%; 36 min) to afford methyl 3-((4-(2-(2-aminopyridin-3-yl)-5-phenyl-3H-imidazo[4,5-b]pyridin-3-yl)benzyl)carbamoyl)-4-fluorobenzoate 22 (32.2 mg, 0.0540 mmol, 10.6% yield) as a light yellow solid. ¹H NMR (400 MHz, DMSO-d₆) δ_{H}=9.15 (t, J=5.6 Hz, 1H), 8.29-8.21 (m, 2H), 8.15-8.09 (m, 1H), 8.05-7.97 (m, 4H), 7.53-7.44 (m, 7H), 7.42-7.36 (m, 1H), 7.24-7.20 (m, 1H), 6.96 (s, 2H), 6.42 (dd, J=4.8, 7.6 Hz, 1H), 4.61 (d, J=6.0 Hz, 2H), 3.88 (s, 3H). HPLC Rₜ=3.096 min in 8 min chromatography, purity 95.9%. LCMS Rₜ=1.377 min in 2 min chromatography, purity 94.9%, MS ESI calcd, for 572.20, [M+H]⁺ 573.20, found 573.0.

### Example 23. Synthesis of 3-((4-(2-(2-aminopyridin-3-yl)-5-phenyl-3H-imidazo[4,5-b]pyridin-3-yl)benzyl)carbamoyl)-4-fluorobenzoic acid (Compound 23)

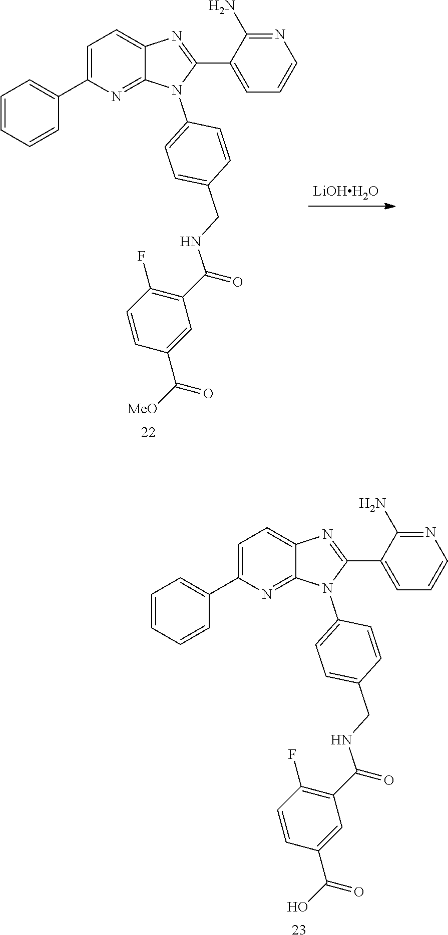

Step 1. To a solution of methyl 3-((4-(2-(2-aminopyridin-3-yl)-5-phenyl-3H-imidazo[4,5-b]pyridin-3-yl)benzyl)carbamoyl)-4-fluorobenzoate 22 (230 mg, 0.402 mmol) in THF (5.00 mL) and water (5.00 mL), LiOH·H₂O (84.0 mg, 2.01 mmol) was added and the mixture stirred at 25° C. for 4 hrs. The resulting mixture was concentrated and the residue was purified by prep-HPLC (Column: Welch Xtimate C18 150*30 mm*5 μm; mobile phase: [water (NH₄HCO₃)-ACN]; B %: 36%-44%; 36 min) to afford 3-((4-(2-(2-aminopyridin-3-yl)-5-phenyl-3H-imidazo[4,5-b]pyridin-3-yl)benzyl)carbamoyl)-4-fluorobenzoic acid 23 (43.8 mg, 0.0784 mmol, 19.5% yield) as a light yellow solid. ¹H NMR (400 MHz, DMSO-d₆) δ_{H}=9.13 (t, J=5.6 Hz, 1H), 8.32-8.19 (m, 2H), 8.13-7.96 (m, 5H), 7.53-7.36 (m, 8H), 7.25-7.19 (m, 1H), 6.97 (s, 2H), 6.42 (dd, J=4.8, 7.6 Hz. 1H), 4.64-4.58 (m, 2H). HPLC Rₜ=2.799 min in 8 min chromatography, purity 99.9%. LCMS Rₜ=1.245 min in 2 min chromatography, purity 99.9%, MS ESI calcd, for 558.18, [M+H]⁺ 559.18, found 559.0.

### Example 24. Synthesis of 3-((4-(2-(2-aminopyridin-3-yl)-5-phenyl-3H-imidazo[4,5-b]pyridin-3-yl)benzyl)carbamoyl)-5-cyanobenzoic acid (Compound 24)

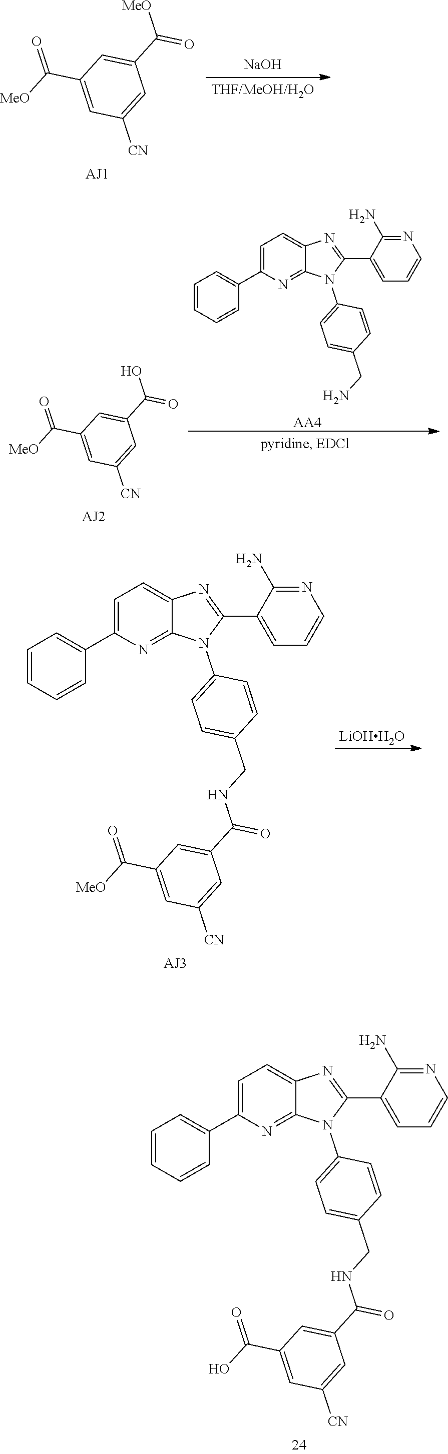

Step 1. To a solution of dimethyl 5-cyanobenzene-1,3-dicarboxylate AJ1 (475 mg, 2.17 mmol) in methanol (2.00 mL), sodium hydroxide (87.0 mg, 2.17 mmol) in THF (2.00 mL) and water (1.00 mL) were added, and the reaction mixture stirred at 25° C. for 12 hrs. The reaction mixture was concentrated to remove THF and then adjusted to pH ~5 by aq. HCl (2 M). The mixture was extracted with EtOAc (20 mL) and washed with brine (2×10 mL). The combined organic layers were dried over Na₂SO₄, filtered and concentrated to give 3-cyano-5-(methoxycarbonyl)benzoic acid AJ2 (603 mg, crude) as a white solid, which was used in the next step without further purification. ¹H NMR (400 MHz, DMSO-d₆) δ_{H}=8.64 (d, J=1.6 Hz, 1H), 8.60-8.49 (m, 2H), 3.92 (d, J=2.0 Hz, 3H).

Step 2. To a solution of 3-(3-(4-(aminomethyl)phenyl)-5-phenyl-3H-imidazo[4,5-b]pyridin-2-yl)pyridin-2-amine AA4 (100 mg, 0.250 mmol) in pyridine (2.00 mL), 3-cyano-5-(methoxycarbonyl)benzoic acid AJ2 (52.0 mg, 0.250 mmol) and EDCI (8.00 mg, 0.210 mmol) were added and the mixture stirred at 25° C. for 2 hrs. The mixture was diluted with H₂O (10 mL), extracted with EtOAc (15 mL×2). The combined organic layer was washed with brine (30 mL), dried over Na₂SO₄, filtered and concentrated. The residue was purified by flash silica gel chromatography (ISCO®; 12 g SepaFlash® Silica Flash Column, Eluent with 71% of Ethyl acetate in Petroleum ether gradient @ 40 mL/min) to give methyl 3-((4-(2~ (2-aminopyridin-3-yl)-5-phenyl-3H-imidazo[4,5-b]pyridin-3-yl)benzyl)carbamoyl)- 5-cyanobenzoate AJ3 (72.0 mg, 48.6% yield) as a yellow solid. LCMS Rₜ=1.297 min in 2.5 min chromatography, purity 88.6%, MS ESI calcd, for 579.20 [M+H]⁺ 580.20, found 580.3.

Step 3. To a solution of methyl 3-((4-(2-(2-aminopyridin-3-yl)-5-phenyl-3H-imidazo[4,5-b]pyridin-3-yl)benzyl)carbamoyl)-5-cyanobenzoate AJ3 (60.0 mg, 0.100 mmol) in THE (2.00 mL), LiOH·H₂O (6.50 mg, 0.150 mmol) and water (1.00 mL) were added, and the reaction mixture was stirred at 25° C. for 2 hrs. The reaction mixture was concentrated to remove THF. The mixture was subsequently diluted with H₂O (10 mL) and extracted with PE (10 mL×3). The pH of the aqueous phase was adjusted to pH ~5 with HCl (2 M). The crude product was purified by prep-HPLC (column: Xtimate C18 150*40 mm*10 μm; mobile phase: water (NH₄HCO₃)-ACN; B %: 4%-44%, 25 min) to give 3-((4-(2-(2-aminopyridin-3-yl)-5-phenyl-3H-imidazo[4,5-b]pyridin-3- yl)benzyl)carbamoyl)-5-cyanobenzoic acid 24 (21.2 mg, 36.2% yield) as a white solid. ¹H NMR (DMSO-d₆, 400 MHz) δ_{H}=9.51 (d, J=5.6 Hz, 1H), 8.73 (s, 1H), 8.51 (s, 1H), 8.41 (s, 1H), 8.25 (d, J=8.4 Hz, 1H), 8.06-7.94 (m, 4H), 7.54-7.43 (m, 6H), 7.39 (d, J=7.2 Hz, 1H), 7.21 (d, J=1.6 Hz, 1H), 6.93 (s, 2H), 6.41 (dd, J=7.6, 4.8 Hz, 1H), 4.63 (d, J=4.4 Hz, 2H). HPLC R: =4.336 min in 8 min chromatography, purity 95.9%. LCMS Rₜ=2.469 min in 4 min chromatography, purity 95.9%, MS ESI calcd. for 565.19 [M+H]+ 566.19, found 566.2.

### Example 25. 3-((4-(2-(2-aminopyridin-3-yl)-5-phenyl-3H-imidazo[4,5-b]pyridin-3-yl)benzyl)carbamoyl)-5-hydroxybenzoic acid (Compound 25)

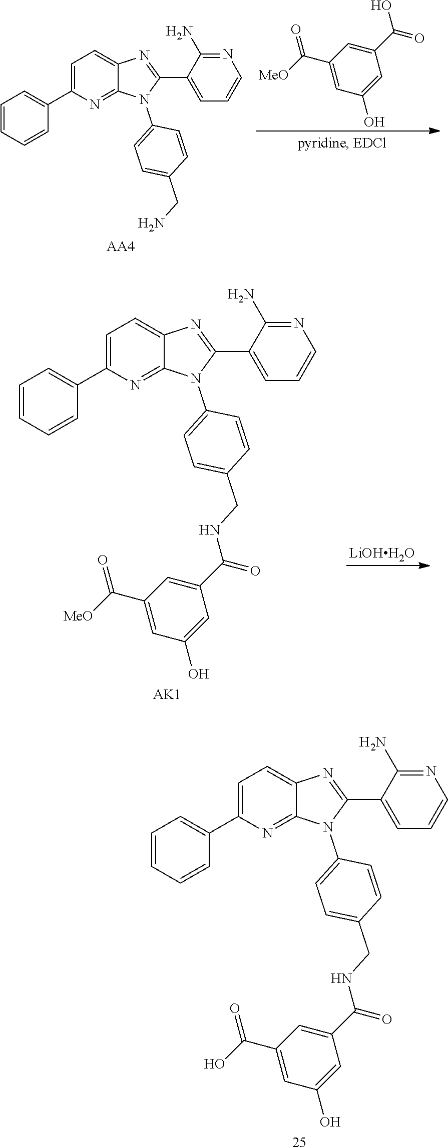

Step 1. To a solution of 3-hydroxy-5-methoxycarbonyl-benzoic acid (50.0 mg, 0.255 mmol) in pyridine (6.00 mL), 3-(3-(4-(aminomethyl)phenyl)-5-phenyl-3H-imidazo[4,5-b]pyridin-2-yl)pyridin-2-amine AA4 (100 mg, 0.255 mmol) and EDCI (73.0 mg, 0.382 mmol) were added at 25° C., and the mixture stirred for 16 hrs. Water (20 mL) was added and the resulting mixture was extracted with EtOAc (10 mL×3). The combined organic phase was washed with brine (5 mL), water (5 mL), dried over anhydrous Na₂SO₄, filtered and concentrated. The residue was purified by flash silica gel chromatography (eluent with 0-10% of MeOH in DCM) to give methyl 3-((4-(2-(2-aminopyridin-3-yl)-5-phenyl-3H-imidazo[4,5-b]pyridin-3-yl)benzyl)carbamoyl)-5-hydroxy benzoate AK1 (100 mg, 68.8% yield) as a yellow solid. ¹H NMR (400 MHz, CHLOROFORM-d) δ=8.62 (d, J=4.4 Hz, 2H), 8.17-8.12 (m, 1H), 8.02-7.93 (m, 4H), 7.81 (d, J=8.0 Hz, 1H), 7.68 (d, J=8.0 Hz, 3H), 7.51 (d, J=6.8 Hz, 2H), 7.47-7.39 (m, 5H), 7.39-7.33 (m, 1H), 7.30 (dd, J=7.2, 6.0 Hz, 2H), 4.81-4.73 (m, 2H), 3.92 (s, 3H).

Step 2. To a solution of methyl 3-((4-(2-(2-aminopyridin-3-yl)-5-phenyl-3H-imidazo[4,5-b]pyridin-3-yl)benzyl)carbamoyl)-5-hydroxybenzoate AK1 (100 mg, 0.175 mmol) in THE (2.00 mL) and water (2.00 mL), LiOH·H₂O (14.0 mg, 0.351 mmol) was added at 25° C. and the mixture stirred for 2 hrs. The reaction mixture was then adjusted to pH ~5 by aq. HCl (1 M). The residue was subsequently purified by prep-HPLC (column: Xtimate C18 150*40 mm*10 μm; condition: water (HCl)-ACN; begin b: 10-50) to give 3-((4-(2-(2-aminopyridin-3-yl)-5-phenyl-3H-imidazo[4,5-b]pyridin-3-yl)benzyl)carbamoyl)-5-hydroxybenzoic acid 25 (19.0 mg, 19.6% yield) as a yellow solid. ¹H NMR (400 MHz, DMSO-d₆) δ=10.11 (s, 1H), 9.27 (t, J=6.0 Hz, 1H), 8.42-8.14 (m, 2H), 8.11-8.02 (m, 4H), 7.97 (s, 1H), 7.87-7.79 (m, 1H), 7.58-7.38 (m, 9H), 6.90-6.80 (m, 1H), 4.58 (d, J=6.0 Hz, 2H). HPLC R: =2.476 min in 8 min chromatography, purity 98.7%. LCMS Rₜ=1.156 min in 2 min chromatography, purity 97.8%, MS ESI calcd, for 556.19, [M+H]⁺ 557.19, found 557.4.

### Example 26. 3-((4-(2-(2-aminopyridin-3-yl)-5-phenyl-3H-imidazo[4,5-b]pyridin-3-yl)benzyl)carbamoyl)-2-hydroxybenzoic acid (Compound 26)

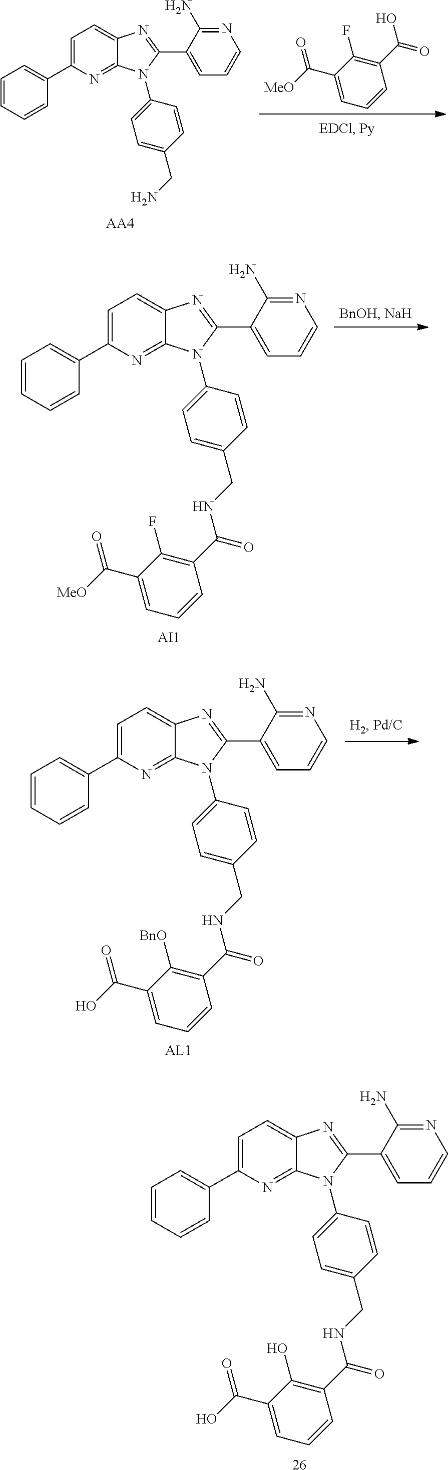

Step 1. To a solution of 2-fluoro-3-methoxycarbonyl-benzoic acid (250 mg, 1.26 mmol) in pyridine (4.00 mL), EDCI (290 mg, 1.51 mmol) and 3-(3-(4-(aminomethyl)phenyl)-5-phenyl-3H-imidazo[4,5-b]pyridin-2-yl)pyridin-2-amine AA4 (396 mg, 1.01 mmol) were added, and the mixture was stirred for 12 hrs. Water (10 mL) was added and the whole extracted with EtOAc (30 mL×3). The organic layer was washed with brine (20 mL) then dried over Na₂SO₄, filtered, and concentrated in vacuum. The crude was purified by flash column (0-20% of MeOH in EtOAc) to give methyl 3-((4-(2-(2-aminopyridin-3-yl)-5-phenyl-3H-imidazo[4,5-b]pyridin-3-yl)benzyl)carbamoyl)-2-fluorobenzoate AI1 (220 mg, 30.5% yield) as a yellow solid. ¹H NMR (400 MHz, DMSO-d₆) δ=9.17 (t, J=6.0 Hz, 1H), 8.27 (d, J=8.0 Hz, 1H), 8.05-7.97 (m, 5H), 7.93-7.85 (m, 1H), 7.52-7.38 (m, 8H), 7.22 (dd, J=7.6, 1.6 Hz, 1H), 6.96 (s, 2H), 6.42 (dd, J=7.6, 4.8 Hz, 1H), 4.60 (d, J=6.0 Hz, 2H), 3.88 (s, 3H). 19F NMR (400 MHz, DMSO-d₆) δ=−113.272.

Step 2. To a solution of phenylmethanol (0.150 mL, 1.48 mmol) in DMF (3.00 mL), NaH (53.0 mg, 1.34 mmol) was added at 0° C. and the mixture stirred for 1 hr. Methyl 3-((4-(2-(2-aminopyridin-3-yl)-5-phenyl-3H-imidazo[4,5-b]pyridin-3-yl)benzyl)carbamoyl)-2-fluoro benzoate AI1 (170 mg, 0.297 mmol) in DMF (3.00 mL) was then added at 0° C., and the mixture was stirred at 25° C. for 12 hrs. Water (10 mL) was added to the reaction mixture, and the whole extracted with EtOAc (30 mL×3). The organic layer was dried over Na₂SO₄, filtered, and concentrated in vacuum. The crude was purified by prep-HPLC (Column: Welch Xtimate C18 150*30 mm*5 μm; Condition: water (FA)-ACN; B %: 28%-58%) to give 3-((4~ (2-(2-aminopyridin-3-yl)-5-phenyl-3H-imidazo[4,5-b]pyridin-3-yl)benzyl)carbamoyl)-2-(benzyloxy)benzoic acid ALI (50.0 mg, 26.0% yield) as a yellow solid. ¹H NMR (400 MHz, DMSO-d₆) δ=8.97 (t, J=6.0 Hz, 1H), 8.28 (d, J=8.4 Hz, 1H), 8.04-7.98 (m, 4H), 7.82 (dd, J=7.6, 1.6 Hz, 1H), 7.71 (dd, J=7.6, 1.6 Hz, 1H), 7.51-7.35 (m, 8H), 7.34-7.16 (m, 7H), 7.12-6.98 (m, 2H), 6.41 (dd, J=7.6, 4.8 Hz, 1H), 5.02 (s, 2H), 4.54 (d, J=6.0 Hz, 2H).

Step 3. To a solution of 3-((4-(2-(2-aminopyridin-3-yl)-5-phenyl-3H-imidazo[4,5-b]pyridin-3-yl)benzyl)carbamoyl)-2-(benzyloxy)benzoic acid ALI (50.0 mg, 0.0773 mmol) in THF (4.00 mL), Pd/C (10.0 mg) was added at 25° C., and the reaction was degassed and purged with Ar (×3), and then degassed and purged with H₂ (×3), and stirred under H₂ (20 Psi) for 16 hrs at 25° C. The reaction mixture was filtered, and the cake was washed with THF (10 mL×3). The combined filtrate was concentrated and purified by prep-HPLC (Column: C18 150×30 mm; Condition: water (HCl)-ACN; B %: 25%-55%) to give 3-((4-(2-(2-aminopyridin-3-yl)-5-phenyl-3H-imidazo[4,5-b]pyridin-3-yl)benzyl)carbamoyl)-2- hydroxybenzoic acid 26 (1.60 mg, 3.72% yield) as a white solid. ¹H NMR (400 MHz, METHANOL-d₄) δ=8.28 (d, J=8.4 Hz, 1H), 8.21 (dd, J=8.0, 2.0 Hz, 1H), 8.12 (dd, J=8.0, 2.0 Hz, 1H), 8.07-7.94 (m, 4H), 7.87-7.82 (m, 1H), 7.65 (d, J=8.4 Hz, 2H), 7.54 (d, J=8.4 Hz, 2H), 7.47-7.36 (m, 3H), 7.08 (t. J=8.0 Hz, 1H), 6.83 (dd, J=7.6, 6.4 Hz, 1H), 2.66 (s, 2H). LCMS Rₜ=2.771 min in 4 min chromatography, purity 95.1%, MS ESI calcd. For 556.19 [M+H]⁺ 557.19, found 557.3. HPLC Rₜ=5.289 min in 8 min chromatography, purity 93.3%

### Example 27. Synthesis of 4-((4-(2-(2-aminopyridin-3-yl)-5-phenyl-3H-imidazo[4,5-b]pyridin-3-yl)benzyl)carbamoyl)-2-(dimethylamino)benzoic acid (Compound 27)

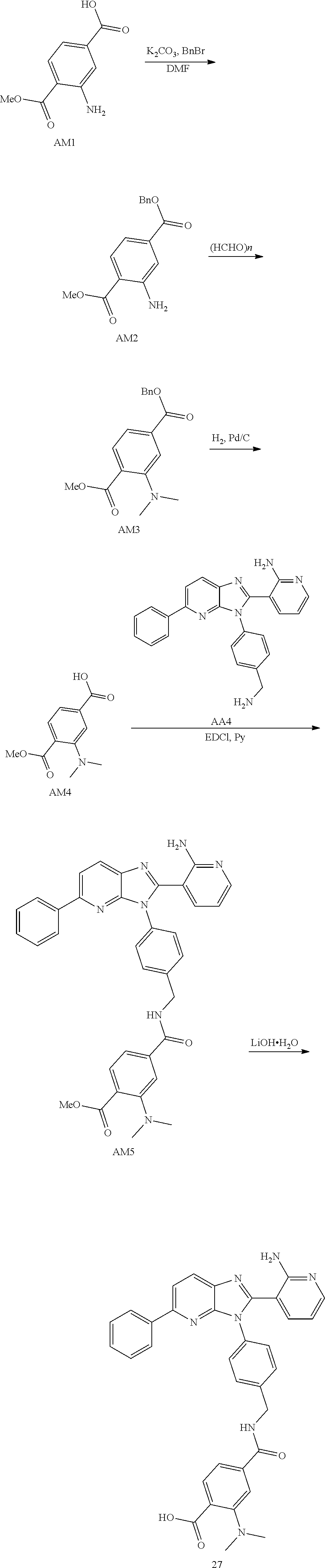

Step 1. To a solution of 3-amino-4-methoxycarbonyl-benzoic acid AM1 (500 mg, 2.56 mmol) in DMF (5.00 mL), BnBr (0.300 mL, 2.56 mmol) and K₂CO₃ (708 mg, 5.12 mmol) were added, and the reaction stirred at 25° C. for 16 hrs. Water (20 mL) was added and the solution extracted with EtOAc (40 mL×3). The organic layer was washed with brine (40 mL), dried over Na₂SO₄, filtered, and concentrated. The crude was purified by flash column (0-50% of EtOAc in PE) to give O4-benzyl O1-methyl 2-aminobenzene-1,4-dicarboxylate AM2 (550 mg, 75.3% yield) as a yellow solid. ¹H NMR (400 MHz, DMSO-d₆) δ_{H}=7.80 (d, J=8.4 Hz, 1H), 7.52-7.28 (m, 6H), 7.10-7.04 (m, 1H), 6.88 (s, 2H), 5.33 (s, 2H), 3.81 (s, 3H).

Step 2. To a solution of O4-benzyl O1-methyl 2-aminobenzene-1,4-dicarboxylate AM2 (450 mg, 1.58 mmol) in formic acid (1.00 mL, 6.31 mmol), formaldehyde (0.360 mL, 4.73 mmol) was added at 25° C., and the reaction mixture stirred at 100° C. for 15 mins. Water (10 mL) was added to the reaction mixture and the whole extracted with EtOAc (30 mL×3). The organic layer was dried over Na₂SO₄, filtered, and concentrated. The crude was purified by flash column (0-30% of EtOAc in PE) to give O4-benzyl O1-methyl 2-(dimethylamino)benzene-1,4-dicarboxylate AM3 (130 mg, 26.3% yield) as a green oil. ¹H NMR (400 MHz, METHANOL-d₄) δ_{H}=7.64 (d, J=6.0 Hz, 2H), 7.50-7.43 (m, 3H), 7.42-7.30 (m, 3H), 5.36 (s, 2H), 3.90 (s, 3H), 2.85 (s, 6H).

Step 3. To a solution of O4-benzyl O1-methyl 2-(dimethylamino)benzene-1,4-dicarboxylate AM3 (140 mg, 0.450 mmol) in ethyl acetate (2.00 mL), Pd/C (40.0 mg, 0.380 mmol) was added at 25° C. The reaction was degassed and purged with Ar (×3), then degassed and purged with H₂ (×3) and stirred under H₂ (20 Psi) at 25° C. for 16 hrs. The reaction mixture was filtered, and the cake was washed with EtOAc (10 mL×3). The combined filtrate was concentrated in vacuum, and the crude material was purified by flash column (0-60% of EtOAc in PE) to give 3-(dimethylamino)-4-methoxycarbonyl-benzoic acid AM4 (45.0 mg, 45.1% yield) as a green solid. ¹H NMR (400 MHz, DMSO-d₆) δ_{H}=7.57 (d, J=8.0 Hz, 1H), 7.47 (d, J=1.2 Hz, 1H), 7.34 (dd, J=8.0, 1.2 Hz, 1H), 3.83 (s, 3H), 2.80 (s, 6H).

Step 4. To a solution of 3-(dimethylamino)-4-methoxycarbonyl-benzoic acid AM4 (45.0 mg, 0.200 mmol) in pyridine (3.00 mL), EDCI (46.0 mg, 0.240 mmol) and 3-(3-(4-(aminomethyl)phenyl)-5-phenyl-3H-imidazo[4,5-b]pyridin-2-yl)pyridin-2-amine AA4 (179 mg, 0.200 mmol) were added and the reaction mixture stirred at 25° C. for 8 hrs. Water (10 mL) was added to the mixture, and the whole extracted with EtOAc (30 mL×3). The combined organic layer was dried over Na₂SO₄, filtered, and concentrated. The crude was purified by flash column (0-30% of MeOH in EtOAc) to give methyl 4-((4-(2-(2-aminopyridin-3-yl)-5-phenyl-3H-imidazo[4,5-b]pyridin-3-yl)benzyl)carbamoyl)-2-(dimethylamino)benzoate AM5 (50.0 mg, 41.5% yield) as a yellow solid. ¹H NMR (400 MHz, DMSO-d₆) δ_{H}=9.22 (t, J=6.0 Hz, 1H), 8.26 (d, J=8.4 Hz, 1H), 8.06-7.94 (m, 4H), 7.60-7.56 (m, 1H), 7.52-7.30 (m, 9H), 7.22 (dd, J=7.6, 1.6 Hz. 1H) 6.94 (s, 1H), 6.42 (dd, J=8.0, 4.8 Hz, 1H), 4.65-4.56 (m, 2H), 3.83 (s, 3H), 2.84-2.80 (m, 6H).

Step 5. To a solution of methyl 4-((4-(2-(2-aminopyridin-3-yl)-5-phenyl-3H-imidazo[4,5-b]pyridin-3-yl)benzyl)carbamoyl)-2-(dimethylamino)benzoate AM5 (50.0 mg, 0.0800 mmol) in THF (1.00 mL) and water (1.00 mL), LiOH·H₂O (7.00 mg, 0.170 mmol) was added and the reaction mixture stirred at 25° C. for 4 hrs. The reaction mixture used directly for purification by prep-HPLC Welch Xtimate C18 150*30 mm*5 μm, water (FA)-ACN to give 4-((4-(2-(2-aminopyridin-3-yl)-5-phenyl-3H-imidazo[4,5-b]pyridin-3-yl)benzyl)carbamoyl)-2-(dimethyl amino)benzoic acid 27 (18.0 mg, 35.6% yield) as a white solid. ¹H NMR (400 MHz, DMSO-d₆) δ_{H}=9.30 (t. J=6.0 Hz, 1H), 8.27 (d, J=8.0 Hz, 1H), 8.08-7.88 (m, 6H), 7.75 (d, J=8.0 Hz, 1H), 7.50-7.43 (m, 6H), 7.42-7.35 (m, 1H), 7.22 (d, J=6.4 Hz, 1H), 6.94 (s, 2H), 6.42 (dd, J=7.6, 4.4 Hz, 1H), 4.63 (d, J=5.6 Hz, 2H), 2.86 (s, 6H). HPLC Rₜ=3.985 min in 8 min chromatography, purity 95.6%. LCMS Rₜ=2.396 min in 4 min chromatography, purity 93.8%, MS ESI calcd. For 583.23 [M+H]⁺ 584.23, found 584.1.

### Example 28. Synthesis of 6-((4-(2-(2-aminopyridin-3-yl)-5-phenyl-3H-imidazo[4,5-b]pyridin-3-yl)benzyl)carbamoyl) picolinic acid (Compound 28)

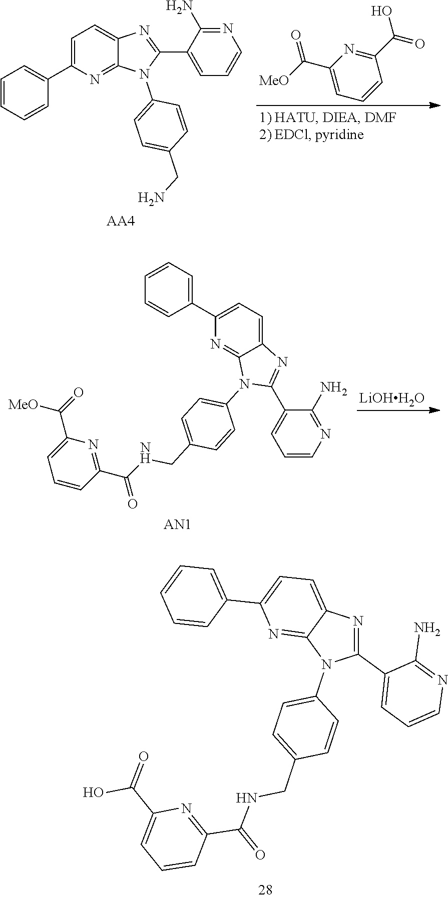

Step 1. To a solution of 6-methoxycarbonylpyridine-2-carboxylic acid (80.0 mg, 0.440 mmol) in DMF (3.00 mL), HATU (174 mg, 0.460 mmol) and DIEA (0.190 mL. 1.15 mmol) were added, and the reaction mixture stirred at 25° C. for 30 mins. 3-(3-(4-(Aminomethyl)phenyl)-5-phenyl-3H-imidazo[4,5-b]pyridin-2-yl)pyridin-2-amine AA4 (173 mg, 0.440 mmol) was subsequently added and the mixture stirred for an additional 12 hrs. EDCI (102 mg, 0.530 mmol) and pyridine (3.00 mL) were added to the mixture and the mixture stirred at 50° C. for 12 hrs. Water (10 mL) was added, and the mixture extracted with EtOAc (30 mL×3). The organic layer was washed with 1 M HCl (10 mL) and brine (10 mL). The organic layer was concentrated, and the crude residue was purified by flash column (0-30% of MeOH in EtOAc) to give methyl 6-((4-(2-(2-aminopyridin-3-yl)-5-phenyl-3H-imidazo[4,5-b]pyridin~3-yl)benzyl)carbamoyl) picolinate AN1 (90.0 mg, 36.7% yield) as a yellow oil. ªH NMR (400 MHz, DMSO-d₆) δ_{H}=9.28 (t, J=6.4 Hz, 1H), 8.35-8.17 (m, 4H), 8.05-7.94 (m, 4H), 7.56-7.34 (m, 7H), 7.22 (dd, J=7.6, 1.6 Hz, 1H), 6.93 (s, 2H), 6.42 (dd, J=8.0, 5.2 Hz, 1H), 4.66 (d, J=6.4 Hz, 2H), 3.95-3.91 (s, 3H).

Step 2. To a solution of methyl 6-((4-(2-(2-aminopyridin-3-yl)-5-phenyl-3H-imidazo[4,5-b]pyridin-3-yl)benzyl)carbamoyl) picolinate AN1 (90.0 mg, 0.160 mmol) in water (2.00 mL) and THE (2.00 mL), LiOH·H₂O (14.0 mg, 0.320 mmol) was added at 25° C. and stirred for 3 hrs. Water (10 mL) was added, and the whole extracted with EtOAc (30 mL×3). The organic layer was dried over Na₂SO₄, filtered, and concentrated. The crude residue was purified by prep-HPLC Welch Xtimate C18 150*30 mm*5 μm water (FA)-ACN to give 6-((4-(2-(2-aminopyridin-3-yl)-5-phenyl-3H-imidazo[4,5-b]pyridin-3-yl)benzyl)carbamoyl) picolinic acid 28 (23.0 mg, 25.9% yield) as a yellow solid. ¹H NMR (400 MHz, DMSO-d₆) δ_{H}=9.86 (t, J=6.4 Hz, 1H), 8.36-8.30 (m, 1H), 8.29-8.20 (m, 3H), 8.06-7.93 (m, 4H), 7.55-7.43 (m, 6H), 7.42-7.34 (m, 1H), 7.23 (dd, J=7.2, 1.6 Hz, 1H), 6.93 (s, 2H), 6.42 (dd, J=7.6, 4.8 Hz, 1H), 4.71 (d, J=6.4 Hz, 2H). HPLC Rₜ=4.330 min in 8 min chromatography, purity 97.9%. LCMS Rₜ=2.573 min in 4 min chromatography, purity 96.6%, MS ESI calcd. For 541.19 [M+H]⁺ 542.19, found 542.1.

### Example 29. Synthesis of methyl 2-(4-((4-(2-(2-aminopyridin-3-yl)-5-phenyl-3H-imidazo[4,5-b]pyridin-3-yl)benzyl)carbamoyl)phenyl)acetate (Compound 29)

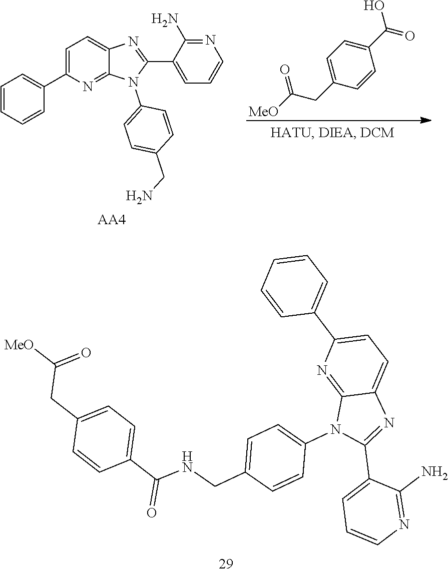

Step 1. To a solution of 3-(3-(4-(aminomethyl)phenyl)-5-phenyl-3H-imidazo[4,5-b]pyridin-2-yl)pyridin-2-amine AA4 (500 mg, 1.27 mmol) in DCM (10.0 mL), 4-(2-methoxy-2-oxo-ethyl)benzoic acid (495 mg, 2.55 mmol), DIEA (494 mg, 3.82 mmol) and HATU (727 mg. 1.91 mmol) were added at 25° C., and the mixture stirred at 25° C. for 3 hrs. The reaction mixture was concentrated directly and the residue was purified by prep-HPLC (Column: Welch Xtimate C18 150*30 mm*5 μm; Condition: water (HCl)-ACN; Begin B: 14-54%) to give methyl 2-(4-((4-(2-(2-aminopyridin-3-yl)-5-phenyl-3H-imidazo[4,5-b]pyridin-3-yl)benzyl)carbamoyl)phenyl)acetate 29 (12.0 mg, 0.0214 mmol, 1.68% yield) as a yellow solid. ¹H NMR (400 MHz, DMSO-d₆) δ=9.15 (t, J=6.0 Hz, 1H), 8.35 (d, J=8.4 Hz, 2H), 8.11-8.03 (m, 4H), 7.92-7.85 (m, 3H), 7.63-7.34 (m, 10H), 6.92-6.83 (m, 1H), 4.60 (d, J=6.0 Hz, 2H), 3.77 (s, 2H), 3.62 (s, 3H). HPLC Rₜ=3.987 min in 8 min chromatography, purity 98.0%. LCMS Rₜ=1.242 min in 2 min chromatography, purity 95.4%, MS ESI calcd. for 568.22 [M+H]⁺ 569.22, found 569.1.

### Example 30. Synthesis of 2-(4-((4-(2-(2-aminopyridin-3-yl)-5-phenyl-3H-imidazo[4,5-b]pyridin-3-yl)benzyl)carbamoyl)phenyl)acetic acid (Compound 30)

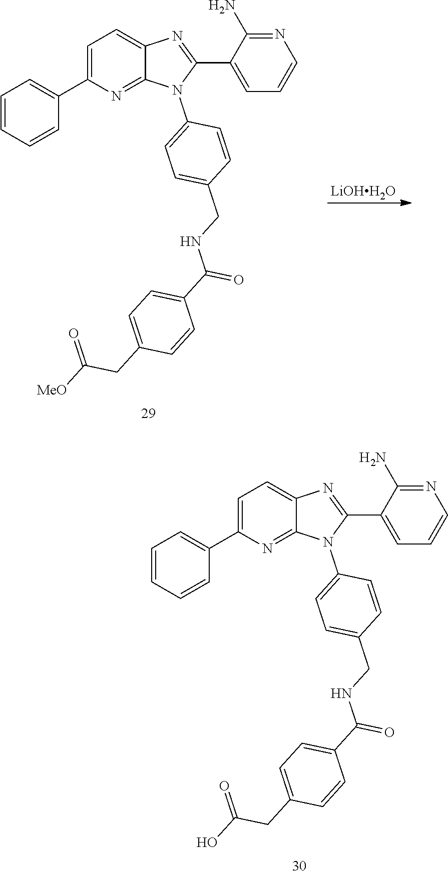 [SMILES: COC(=O)Cc1ccc(C(=O)NCc2ccc(-n3c(-c4cccnc4N)nc4ccc(-c5ccccc5)nc43)cc2)cc1 ; "LiOH·H2O" ; Nc1ncccc1-c1nc2ccc(-c3ccccc3)nc2n1-c1ccc(CNC(=O)c2ccc(CC(=O)O)cc2)cc1]

Step 1. To a solution of methyl 2-(4-((4-(2-(2-aminopyridin-3-yl)-5-phenyl-3H-imidazo [4,5-b]pyridin-3-yl)benzyl)carbamoyl)phenyl)acetate 29 (100 mg, 0.176 mmol) in THF (2.00 mL) and water (2.00 mL), LiOH·H₂O (22.0 mg, 0.528 mmol) was added at 25° C. and the mixture stirred for 2 hrs. The reaction mixture was concentrated to remove THF and the resulting residue was purified by prep-HPLC (Column: Welch Xtimate C18 150*30 mm*5 μm; Condition: water (HCl)-ACN; Begin B: 10-50%) to give 2-(4-((4-(2-(2-aminopyridin-3-yl)-5-phenyl-3H-imidazo[4,5-b]pyridin-3-yl)benzyl)carbamoyl)phenyl)acetic acid 30 (41.0 mg, 0.0745 mmol, 42.3% yield) as a yellow solid. ¹H NMR (400 MHz, DMSO-d₆) δ=9.13 (t, J=6.0 Hz, 1H), 8.39-8.17 (m, 2H), 8.11-8.02 (m, 4H), 7.91-7.80 (m, 3H), 7.56-7.45 (m, 6H), 7.44-7.35 (m, 3H), 6.85 (dd, J=7.2, 6.4 Hz, 1H), 4.60 (d, J=6.0 Hz, 2H), 3.65 (s, 2H). HPLC Rₜ=2.452 min in 8 min chromatography, purity 100.00%. LCMS Rₜ=1.164 min in 2 min chromatography, purity 95.96%, MS ESI calcd, for 554.21 [M+H]⁺ 555.21, found 555.2.

### Example 31. Synthesis of methyl 2-(3-((4-(2-(2-aminopyridin-3-yl)-5-phenyl-3H-imidazo[4,5-b]pyridin-3-yl)benzyl)carbamoyl)phenyl)acetate (Compound 31)

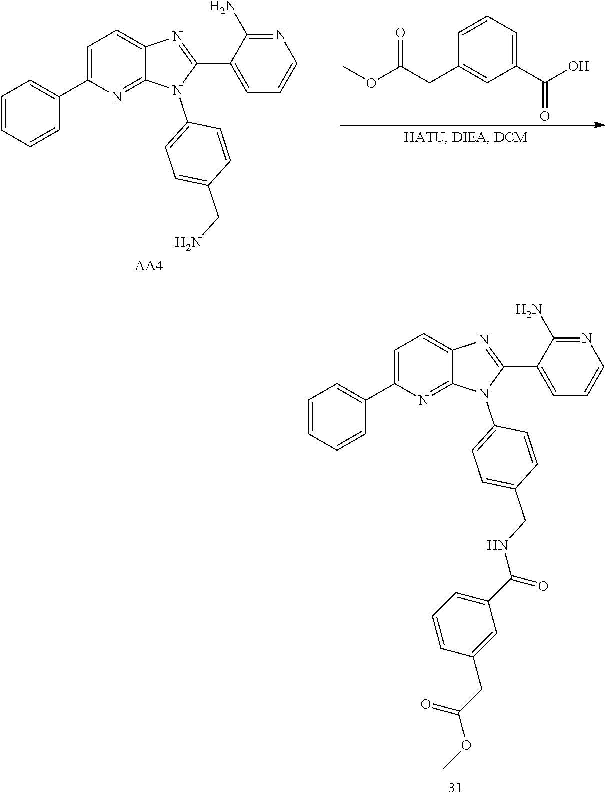

Step 1. To a solution of 3-(3-(4-(aminomethyl)phenyl)-5-phenyl-3H-imidazo[4,5-b]pyridin-2-yl)pyridin-2-amine AA4 (400 mg, 1.02 mmol) in DCM (10.0 mL), 3-(2-methoxy-2-oxo-ethyl)benzoic acid (218 mg, 1.12 mmol), DIEA (395 mg, 3.06 mmol) and HATU (581 mg. 1.53 mmol) were added at 25° C. and the mixture stirred for 3 hrs. The reaction mixture was concentrated directly and the residue was purified by prep-HPLC (Column: Welch Xtimate C18 150*30 mm*5 μm; Condition: water (HCl)-ACN; Begin B: 16-56%) to give methyl 2-(3-((4-(2-(2-aminopyridin-3-yl)-5-phenyl-3H-imidazo[4,5-b]pyridin-3-yl)benzyl)carbamoyl)phenyl)acetate 31 (72.0 mg, 0.125 mmol, 12.3% yield) as a yellow solid. ¹H NMR (400 MHz, DMSO-d₆) δ=9.19-9.12 (m, 1H), 8.38-8.17 (m, 2H), 8.10-8.02 (m, 4H), 7.89-7.80 (m, 3H), 7.56-7.40 (m, 9H), 6.87 (t, J=6.0 Hz, 1H), 4.60 (d, J=6.0 Hz, 2H), 3.77 (s, 2H), 3.63 (s, 3H). HPLC Rₜ=2.708 min in 8 min chromatography, purity 98.7%. LCMS Rₜ=1.233 min in 2 min chromatography, purity 98.7%, MS ESI calcd, for 568.22 [M+H]⁺ 569.22, found 569.1.

### Example 32. Synthesis of 2-(3-((4-(2-(2-aminopyridin-3-yl)-5-phenyl-3H-imidazo[4,5-b]pyridin-3-yl)benzyl)carbamoyl)phenyl)acetic acid (Compound 32)

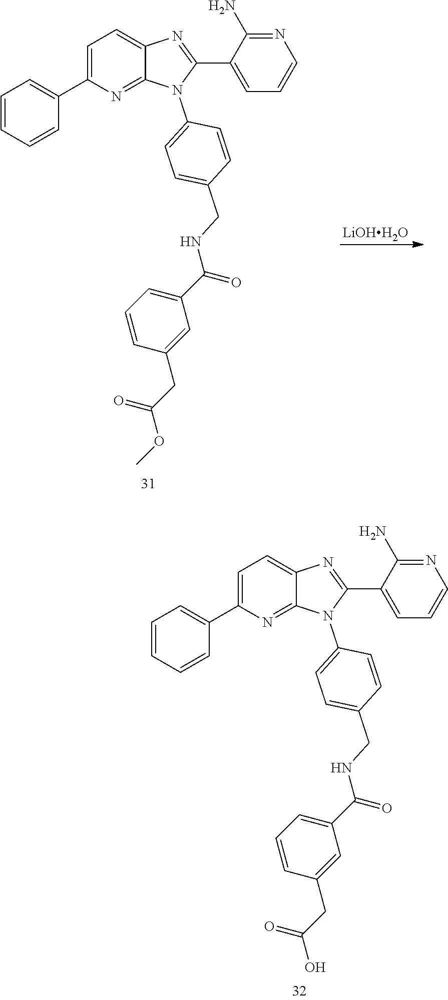 [SMILES: COC(=O)Cc1cccc(C(=O)NCc2ccc(-n3c(-c4cccnc4N)nc4ccc(-c5ccccc5)nc43)cc2)c1 ; "LiOH·H2O" ; Nc1ncccc1-c1nc2ccc(-c3ccccc3)nc2n1-c1ccc(CNC(=O)c2cccc(CC(=O)O)c2)cc1]

Step 1. To a solution of methyl 2-(3-((4-(2-(2-aminopyridin-3-yl)-5-phenyl-3H-imidazo[4,5-b]pyridin-3-yl)benzyl)carbamoyl)phenyl)acetate 31 (100 mg, 0.176 mmol) in THF (2.00 mL) and water (2.00 mL), LiOH·H₂O) (22.0 mg, 0.528 mmol) was added at 25° C. and the mixture was stirred for 2 hrs. The reaction mixture was concentrated to remove THE and the residue was purified by prep-HPLC (Column: Welch Xtimate C18 150*30 mm*5 μm; Condition: water (HCl)-ACN; Begin B: 10-50%) to give 2-(3-((4-(2-(2-aminopyridin-3-yl)-5-phenyl-3H-imidazo[4,5-b]pyridin-3- yl)benzyl)carbamoyl)phenyl)acetic acid 32 (29.0 mg, 0.0514 mmol, 29.2% yield) as a yellow solid. ¹H NMR (400 MHz, DMSO-d₆) δ=9.17 (t, J=6.0 Hz, 1H), 8.51-8.30 (m, 2H), 8.11 (d, J=6.4 Hz, 1H), 8.05 (dd, J=8.4, 4.0 Hz, 3H), 7.89 (d, J=7.2 Hz, 1H), 7.83 (s, 2H), 7.57-7.40 (m, 8H), 6.89 (t, J=6.8 Hz, 1H), 4.60 (d, J=6.0 Hz, 2H), 3.65 (s, 2H). HPLC Rₜ=2.438 min in 8 min chromatography, purity 100%. LCMS Rₜ=1.141 min in 2 min chromatography, purity 96.8%, MS ESI calcd, for 554.21 [M+H]+ 555.21, found 555.1.

### Example 33. Synthesis of methyl 2-(3-((4-(2-(2-aminopyridin-3-yl)-5-phenyl-3H-imidazo[4,5-b]pyridin-3-yl)benzyl)carbamoyl)-2-fluorophenyl)acetate (Compound 33)

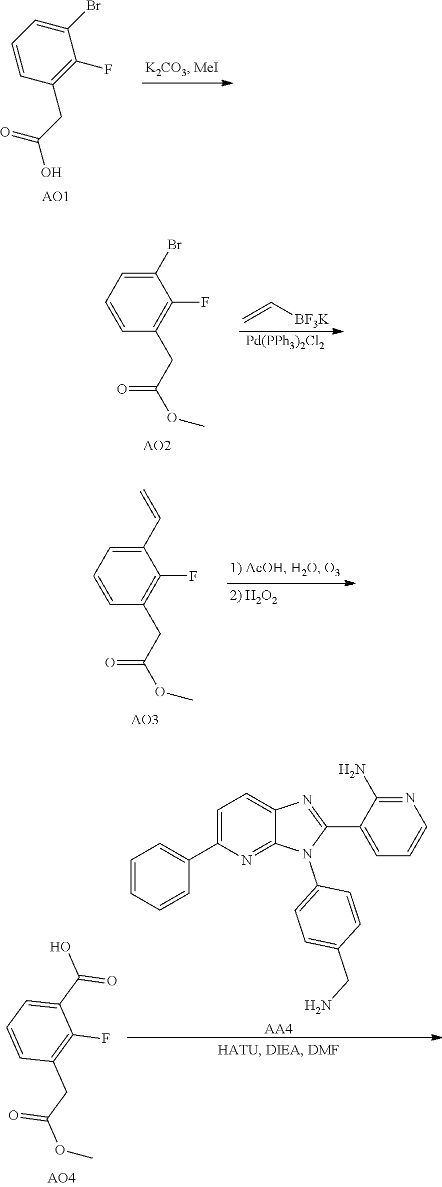

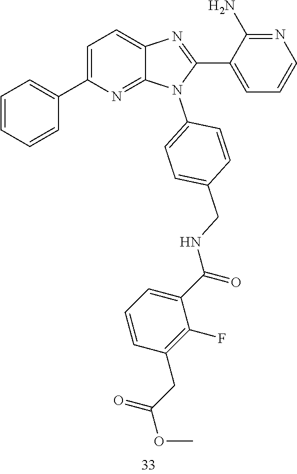 [SMILES: COC(=O)Cc1cccc(C(=O)NCc2ccc(-n3c(-c4cccnc4N)nc4ccc(-c5ccccc5)nc43)cc2)c1F]

Step 1. To a solution of 2-(3-bromo-2-fluorophenyl)acetic acid A⁰¹ (3.00 g, 12.9 mmol) and K₂CO₃ (5.34 g, 38.6 mmol) in DMF (40.0 mL), CHI (3.65 g, 25.7 mmol) was added and the mixture stirred at 25° C. for 12 hrs. Water (5 mL) was added, and the mixture was extracted with EtOAc (5 mL×3). The combined organic phase was washed with brine (5 mL), dried over anhydrous Na₂SO₄, filtered and concentrated. The residue was purified by flash silica gel chromatography (eluent with 0-10% of EtOAc in PE) to give methyl 2-(3-bromo-2-fluorophenyl)acetate AO2 (2.03 g, 8.22 mmol, 63.8% yield) as a colorless oil. ¹H NMR (DMSO-d₆, 400 MHz) δ_{H}=7.67-7.57 (m, 1H), 7.41-7.33 (m, 1H), 7.17-7.10 (m, 1H), 3.82-3.79 (m, 2H), 3.64 (s, 3H).

Step 2. To a solution of methyl 2-(3-bromo-2-fluorophenyl)acetate A⁰² (500 mg, 2.02 mmol) and potassium vinyltrifluoroborate (352 mg, 2.63 mmol) in 1,4-dioxane (10.0 mL) and water (2.00 mL), Cs₂CO₃ (1.98 g, 6.07 mmol) and Pd(dppf)Cl₂ (147 mg, 0.202 mmol) were added and the mixture stirred at 100° C. for 12 hrs. The reaction mixture was concentrated. Water (5 mL) was then added to the residue and the resulting mixture was extracted with BtOAc (10 mL×3) and the combined organic phase was washed with brine (10 mL), dried over anhydrous Na₂SO₄, filtered and concentrated. The residue was purified by flash silica gel chromatography (eluent with 0-10% of EtOAc in PE) to give methyl 2-(2-fluoro-3-vinylphenyl)acetate AO3 (400 mg, 1.96 mmol, 96.7% yield) as a colorless oil. ¹H NMR (DMSO-d₆, 400 MHz) δ_{H}=7.60-7.53 (m, 1H), 7.31-7.23 (m, 1H), 7.18-7.12 (m, 1H), 6.83 (dd, J=11.2, 17.6 Hz, 1H), 5.95-5.87 (m, 1H), 5.47-5.39 (m, 1H), 3.76-3.73 (m, 2H), 3.63 (s, 3H).

Step 3. A solution of methyl 2-(2-fluoro-3-vinylphenyl)acetate A⁰³ (600 mg, 3.09 mmol) in acetic acid (6.00 mL) and water (6.00 mL) was stirred at 0° C. under 03 for 0.5 hrs. H₂O₂ (0.710 mL, 29.4 mmol) was then added into the mixture under N₂, and the mixture was stirred at 50° C. for 2 hrs. Aq. Na₂SO₃ (5 mL) was added into the mixture, and the resulting mixture was extracted with EtOAc (5 mL×3). The combined organic phase was washed with brine (5 mL), dried over anhydrous Na₂SO₄, filtered and concentrated. The residue was purified by prep-HPLC (Column: Welch Xtimate C18 150*30 mm*5 μm; mobile phase: [water (HCl)-ACN]; B %: 36%-44%; 36 min) to give 2-fluoro-3-(2-methoxy-2-oxoethyl)benzoic acid A⁰⁴ (155 mg, 0.731 mmol, 23.7% yield) as a white solid. ¹H NMR (DMSO-d₆, 400 MHz) δ_{H}=13.23 (s, 1H), 7.81-7.73 (m, 1H), 7.62-7.54 (m, 1H), 7.25 (t, J=7.6 Hz, 1H), 3.79 (s, 2H), 3.64 (s, 3H).

Step 4. To a solution of 3-(3-(4-(aminomethyl)phenyl)-5-phenyl-3H-imidazo[4,5-b]pyridin-2-yl)pyridin-2-amine AA4 (200 mg, 0.510 mmol) and 2-fluoro-3-(2-methoxy-2-oxoethyl)benzoic acid A⁰⁴ (108 mg, 0.510 mmol) in DMF (5.00 mL), DIEA (198 mg, 1.53 mmol) and HATU (233 mg, 0,612 mmol) were added and the mixture stirred at 25° C. for 4 hrs. Water (5 mL) was added to the reaction mixture and the resulting mixture was extracted with EtOAc (10 mL×3). The combined organic phase was washed with brine (10 mL), dried over anhydrous Na₂SO₄, filtered and concentrated. The residue was purified by flash silica gel chromatography (eluent with 0-5% of MeOH in DCM) to afford methyl 2-(3-((4-(2-(2-aminopyridin-3-yl)-5-phenyl-3H-imidazo[4,5-b]pyridin-3-yl)benzyl)carbamoyl)-2-fluorophenyl)acetate 33 (39.0 mg, 0.0657 mmol, 12.9% yield) as a light yellow solid. ¹H NMR (DMSO-d₆, 400 MHz) δ_{H}=9.02 (t, J=5.2 Hz, 1H), 8.27 (d, J=8.4 Hz, 1H), 8.06-7.97 (m, 4H), 7.62-7.57 (m, 1H), 7.52-7.44 (m, 7H), 7.42-7.36 (m, 1H), 7.28-7.21 (m, 2H), 7.00 (s, 2H), 6.43 (dd, J=4.8, 7.6 Hz, 1H), 4.58 (d, J=6.0 Hz, 2H), 3.80 (s, 2H), 3.64 (s, 3H). HPLC Rₜ=2.917 min in 8 min chromatography, purity 98.8%. LCMS Rₜ=1.311 min in 2 min chromatography, purity 99.1%, MS ESI calcd, for 586.21, [M+H]⁺ 587.21, found 587.0.

### Example 34. Synthesis of 2-(3-((4-(2-(2-aminopyridin-3-yl)-5-phenyl-3H-imidazo[4,5-b]pyridin-3-yl)benzyl)carbamoyl)-2-fluorophenyl)acetic acid (Compound 34)

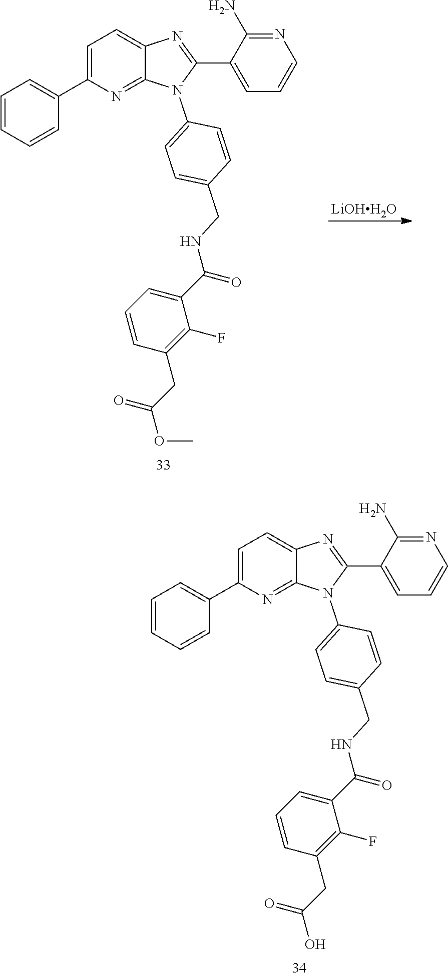 [SMILES: COC(=O)Cc1cccc(C(=O)NCc2ccc(-n3c(-c4cccnc4N)nc4ccc(-c5ccccc5)nc43)cc2)c1F ; "LiOH·H2O" ; Nc1ncccc1-c1nc2ccc(-c3ccccc3)nc2n1-c1ccc(CNC(=O)c2cccc(CC(=O)O)c2F)cc1]

Step 1. To a solution of methyl 2-(3-((4-(2-(2-aminopyridin-3-yl)-5-phenyl-3H-imidazo[4,5-b]pyridin-3-yl)benzyl)carbamoyl)-2-fluorophenyl)acetate 33 (150 mg, 0.256 mmol) in THF (3.00 mL) and water (3.00 mL). LiOH·H₂O (54.0 mg, 1.28 mmol) was added and the mixture stirred at 25° C. for 4 hrs. The reaction mixture was concentrated directly and the crude residue was purified by prep-HPLC (Column: Welch Xtimate C18 150*30 mm*5 μm, mobile phase: [water (HCl)-ACN]; B %: 36%-44%; 36 min) to afford 2-(3-(4-(2-(2-aminopyridin-3-yl)-5-phenyl-3H-imidazo[4,5-b]pyridin-3-yl)benzyl)carbamoyl)-2-fluorophenyl)acetic acid 34 (76.5 mg, 0.132 mmol, 51.6% yield) as a light yellow solid. ¹H NMR (DMSO-d₆, 400 MHz) δ_{H}=9.01 (t, J=5.2 Hz, 1H), 8.27 (d, J=8.4 Hz, 1H), 8.07-7.95 (m, 4H), 7.60-7.53 (m, 1H), 7.52-7.43 (m, 7H), 7.41-7.36 (m, 1H), 7.26-7.19 (m, 2H), 6.97 (s, 2H), 6.42 (dd, J=4.8, 7.6 Hz, 1H), 4.58 (d, J=6.0 Hz, 2H), 3.67 (s, 2H). HPLC Rₜ=2.649 min in 8 min chromatography, purity 98.7%. LCMS Rₜ=1.180 min in 2 min chromatography, purity 98.4%, MS ESI calcd, for 572.20, [M+H]⁺ 573.20, found 572.9.

### Example 35. Synthesis of 2-(4-((4-(2-(2-aminopyridin-3-yl)-5-phenyl-3H-imidazo[4,5-b]pyridin-3-yl)benzyl)carbamoyl)phenyl) propanoic acid (Compound 35)

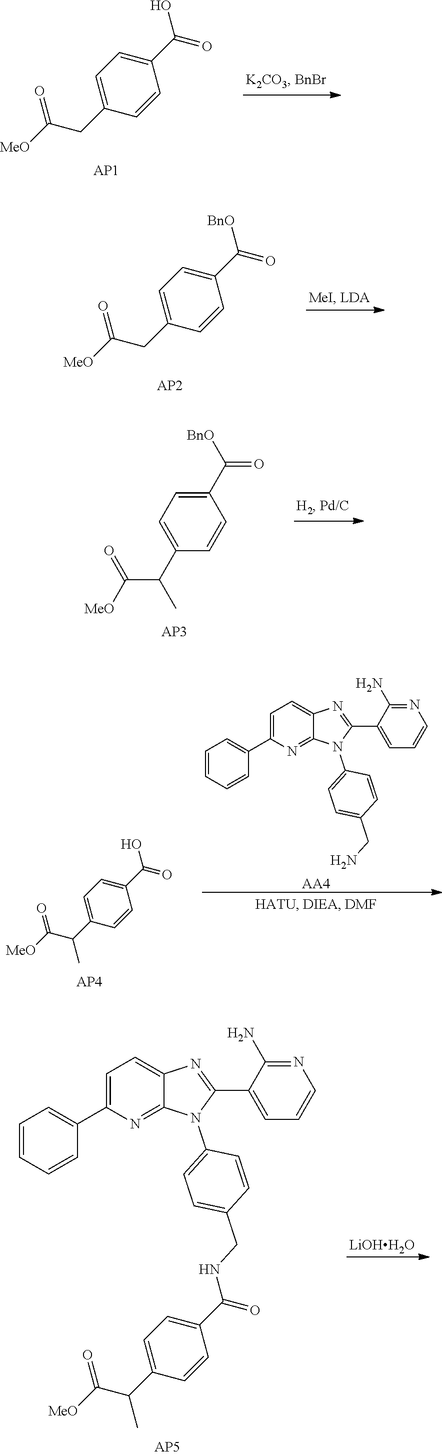

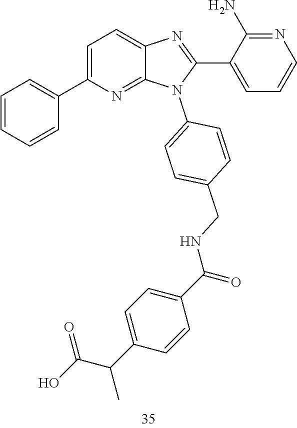 [SMILES: CC(C(=O)O)c1ccc(C(=O)NCc2ccc(-n3c(-c4cccnc4N)nc4ccc(-c5ccccc5)nc43)cc2)cc1]

Step 1. To a solution of 4-(2-methoxy-2-oxoethyl)benzoic acid AP1 (500 mg, 2.57 mmol) in DMF (5.00 mL), K₂CO₃ (427 mg, 3.09 mmol) and BnBr (396 mg, 2.32 mmol) were added and the mixture stirred at 25° C. for 0.5 hr. Water (20 mL) was added to the residue and the resulting mixture was extracted with EtOAc (25 mL×2). The combined organic phase was washed with brine (30 mL×4), dried over anhydrous Na₂SO₄, filtered and concentrated.

The residue was purified by flash silica gel chromatography (eluent of 4% EtOAc in PE) to give benzyl 4-(2-methoxy-2-oxoethyl)benzoate AP2 (700 mg, 95.6% yield) as a colorless oil. ¹H NMR (400 MHz, DMSO-d₆) δ_{H}=7.98-7.92 (m, 2H), 7.51-7.30 (m, 7H), 5.35 (s, 2H), 3.80 (s, 2H), 3.62 (s, 3H).

Step 2. To a solution of benzyl 4-(2-methoxy-2-oxoethyl)benzoate AP2 (700 mg, 2.46 mmol) in THF (15.0 mL), LDA (316 mg, 2.95 mmol) (2M, 1.5 mL) was added at −78° C., and the mixture was stirred at −78° C. for 1 hr. MeI (699 mg, 4.92 mmol) was then added to the mixture dropwise at −78° C., the mixture was degassed and purged with N₂ (×3), and then the mixture was stirred at −78° C. for 0.5 hr under N₂ atmosphere. The mixture was then allowed to warm up to 25° C. and stirred for an additional 12 hrs. Water (20 mL) was added to the residue, and the resulting mixture was extracted with EtOAc (25 mL×2). The combined organic phase was washed with brine (30 mL), dried over anhydrous Na₂SO₄, filtered and concentrated. The residue was purified by flash silica gel chromatography (eluent with 4% of EtOAc in PE) to give benzyl 4-(1-methoxy-1-oxopropan-2-yl)benzoate AP3 (300 mg, 40.8% yield) as a colorless oil. ¹H NMR (400 MHz, DMSO-d₆) δ_{H}=7.98-7.93 (m, 2H), 7.48-7.32 (m, 7H), 5.35 (s, 2H), 4.00-3.89 (m, 1H), 3.59 (s, 3H), 1.40 (d, J=7.2 Hz, 3H).

Step 3. To a solution of benzyl 4-(1-methoxy-1-oxopropan-2-yl)benzoate AP3 (300 mg, 1.01 mmol) in THF (10.0 mL), Pd/C (200 mg, wet, 10%) was added, the reaction mixture was degassed and purged with H₂ (×3), and the mixture stirred at 25° C. for 12 hrs under H₂ (15 Psi). The mixture was filtered through celite and washed with methanol/DCM (MeOH/DCM=1/5, 50 ml×4). The filtrate was concentrated to give 4-(1-methoxy-1-oxopropan-2-yl)benzoic acid AP4 (240 mg) as a white solid. ¹H NMR (400 MHz, DMSO-d₆) δ_{H}=12.93 (s, 1H), 7.93-7.86 (m, 2H), 7.43-7.36 (m, 2H), 3.97-3.86 (m, 1H), 3.59 (s, 3H), 1.40 (d, J=7.2 Hz, 3H).

Step 4. To a solution of 3-(3-(4-(aminomethyl)phenyl)-5-phenyl-3H-imidazo[4,5-b]pyridin-2-yl)pyridin-2-amine AA4 (100 mg, 0.250 mmol) in DMF (3.00 mL), 4-(1-methoxy-1-oxopropan-2-yl)benzoic acid AP4 (58.0 mg, 0.280 mmol) and DIEA (99.0 mg, 0.760 mmol) were added. HATU (145 mg, 0.380 mmol) was then added to the mixture, and the mixture was stirred at 25° C. for 2 hrs. Water (30 mL) was added to the mixture, and the whole was extracted with EtOAc (20 mL×2). The combined organic phase was washed with brine (30 mL), dried over anhydrous Na₂SO₄, filtered and concentrated. The residue was purified by combi-flash column (EtOAc in PE=40%) to give methyl 2-(4-((4-(2-(2-aminopyridin-3-yl)-5-phenyl-3H-imidazo[4,5-b]pyridin-3-yl)benzyl)carbamoyl)phenyl) propanoate AP5 (80.0 mg, 37.4%) as a yellow solid. ¹H NMR (400 MHz, DMSO-d₆) δ_{H}=9.17-9.07 (m, 1H), 8.26 (d, J=8.0 Hz, 1H), 8.04-7.96 (m, 4H), 7.90-7.86 (m, 2H), 7.49-7.43 (m, 6H), 7.41-7.36 (m, 3H), 7.21 (dd, J=2.0, 7.6 Hz, 1H), 6.94 (s, 2H), 6.42 (dd, J=4.8, 8.0 Hz, 1H), 4.60 (d, J=6.0 Hz, 2H), 3.93-3.85 (m, 1H), 3.59 (s, 3H), 1.41 (d, J=7.2 Hz, 3H).

Step 5. To a solution of methyl 2-(4-((4-(2-(2-aminopyridin-3-yl)-5-phenyl-3H-imidazo[4,5-b]pyridin-3-yl)benzyl)carbamoyl)phenyl) propanoate AP5 (80.0 mg, 0.140 mmol) in THF (2.00 mL) and water (1.00 mL), LiOH·H₂O (24.0 mg, 0.560 mmol) was added and the mixture stirred at 25° C. for 2 hrs. The reaction mixture was diluted with DMF (4 mL) and purified by prep-HPLC (Column: Welch Xtimate C18 150*40 mm*5 μm; mobile phase: [water (FA)-ACN]; B %: 8%-48%; 25 min) to give 2-(4-((4-(2-(2-aminopyridin-3-yl)-5-phenyl-3H-imidazo[4,5-b]pyridin-3-yl)benzyl)carbamoyl)phenyl) propanoic acid 35 (23.2 mg, 29.7% yield) as a white solid. ¹H NMR (400 MHz, DMSO-d₆) δ_{H}=12.51-12.26 (m, 1H), 9.11 (t, J=6.0 Hz, 1H), 8.26 (d, J=8.4 Hz, 1H), 8.08-7.95 (m, 4H), 7.92-7.86 (m, 2H), 7.50-7.42 (m, 6H), 7.42-7.36 (m, 3H), 7.21 (dd, J=2.0, 7.6 Hz, 1H), 6.94 (s, 2H), 6.42 (dd, J=4.8, 8.0 Hz, 1H), 4.61 (d, J=6.0 Hz, 2H), 3.76 (q, J=7.2 Hz, 1H), 1.38 (d, J=7.2 Hz, 3H). HPLC Rₜ=2.809 min in 8 min chromatography, purity 95.8%. LCMS Rₜ=1.693 min in 4 min; Agilent PoroShell 120 EC-C18 2.7 μm 3.0*50 mm; purity 97.4%, MS ESI calcd, for 568.22, [M+H]⁺ 569.22, found 569.2.

### Example 36. Synthesis of 2-(3-((4-(2-(2-aminopyridin-3-yl)-5-phenyl-3H-imidazo[4,5-b]pyridin-3-yl)benzyl)carbamoyl)phenyl) propanoic acid (Compound 36)

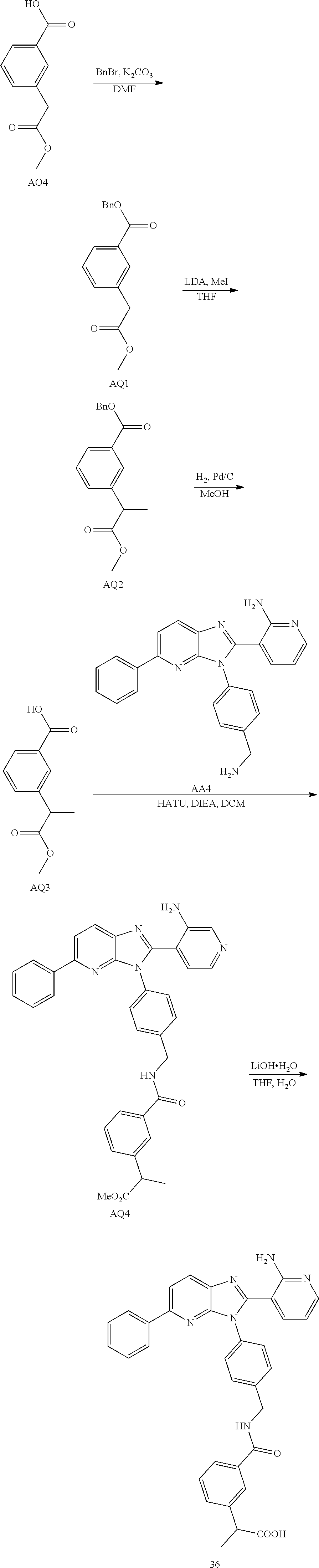

Step 1. To a solution of 3-(2-methoxy-2-oxoethyl)benzoic acid A⁰⁴ (500 mg, 2.57 mmol) in DMF (10.0 mL), K₂CO₃ (430 mg, 3.09 mmol) and BnBr (400 mg, 2.32 mmol) were added and the mixture stirred at 25° C. for 2 hrs. Water (5 mL) was added to the residue and the resulting mixture was extracted with EtOAc (10 mL×3). The combined organic phase was washed with brine (10 mL), dried over anhydrous Na₂SO₄, filtered and concentrated. The residue was purified by flash silica gel chromatography (eluent with 0-5% of EtOAc in PE) to give benzyl 3-(2-methoxy-2-oxoethyl)benzoate AQ1 (545 mg, 1.92 mmol, 74.5% yield) as a light yellow oil. ¹H NMR (400 MHz, DMSO-d₆) δ=7.98-7.86 (m, 2H), 7.61-7.26 (m, 7H), 5.36 (s, 2H), 3.80 (s, 2H), 3.62 (s, 3H).

Step 2. To a solution of benzyl 3-(2-methoxy-2-oxoethyl)benzoate AQ1 (495 mg, 1.74 mmol) in THF (10.0 mL), LDA (1.00 mL, 2.09 mmol, 2 M) was added dropwise at −78° C., and the mixture was stirred at −78° C. for 0.5 hr. A solution of MeI (494 mg, 3.48 mmol) in THF (2.00 mL) was then added at −78° C., and the mixture was stirred at −78° C. for 1 hr, and subsequently stirred at 25° C. for 16 hrs. The reaction was quenched with sat. aq. NH₄Cl (5 mL) drop-wise, and the resulting mixture was extracted with EtOAc (10 mL×3). The combined organic phase was washed with brine (5 mL), dried over anhydrous Na₂SO₄, filtered and concentrated to give benzyl 3-(1-methoxy-1-oxopropan-2-yl)benzoate AQ2 (200 mg, crude) as a yellow oil. ¹H NMR (400 MHz, DMSO-d₆) δ=7.92-7.87 (m, 2H), 7.61-7.56 (m, 1H), 7.53-7.32 (m, 6H), 5.36 (s, 2H), 4.00-3.90 (m, 1H), 3.60-3.56 (m, 3H), 1.41 (d, J=7.2 Hz, 3H).

Step 3. To a solution of benzyl 3-(1-methoxy-1-oxopropan-2-yl)benzoate A Q2 (200 mg, 0.670 mmol) in methanol (5.00 mL), Pd/C (wet, 10%, 0.200 g) was added under a N atmosphere, and the suspension was degassed and purged with H₂ (×3). The reaction mixture was subsequently stirred under H₂ (15 Psi) at 25° C. for 12 hrs. The reaction mixture was filtered and the filtrate was concentrated to give 3-(1-methoxy-1-oxopropan-2-yl)benzoic acid AQ3 (145 mg, crude) as a yellow oil. ¹H NMR (400 MHz, DMSO-d₆) δ=7.91-7.79 (m, 2H), 7.58-7.43 (m, 2H), 3.92 (d, J=6.8 Hz, 1H), 3.59 (s, 3H), 1.41 (d, J=7.2 Hz, 3H).

Step 4. To a solution of 3-(3-(4-(aminomethyl)phenyl)-5-phenyl-3H-imidazo[4,5-b]pyridin-2-yl)pyridin-2-amine AA4 (200 mg, 0.510 mmol) in DCM (10.0 mL), DIEA (200 mg, 1.53 mmol) was added. 3-(1-Methoxy-1-oxopropan-2-yl)benzoic acid AQ3 (140 mg, 0.665 mmol) and HATU (230 mg, 0.612 mmol) were then added, and the mixture stirred at 25° C. for 2 hrs. The reaction mixture was concentrated directly, and the residue was purified by flash silica gel chromatography (eluent with 0-5% of MeOH in DCM) to give methyl 2-(3-((4-(2-(2-aminopyridin-3-yl)-5-phenyl-3H-imidazo[4,5-b]pyridin~3-yl)benzyl)carbamoyl)phenyl) propanoate AQ4 (310 mg, 0.532 mmol) as a yellow solid. ¹H NMR (400 MHz, DMSO-d₆) δ=9.21-9.17 (m, 1H), 8.49 (br s, 5H), 8.27 (d, J=8.4 Hz, 1H), 8.03-7.99 (m, 3H), 7.86-7.83 (m, 2H), 7.48-7.36 (m, 7H), 7.29-7.26 (m, 1H), 6.49-6.42 (m, 1H), 4.61 (d, J=6.0 Hz, 2H), 3.92-3.87 (m, 1H), 3.59 (s, 3H), 1.43 (d, J=6.8 Hz, 3H).

Step 5. To a solution of methyl 2-(3-((4-(2-(2-aminopyridin-3-yl)-5-phenyl-3H-imidazo[4,5-b]pyridin-3-yl)benzyl)carbamoyl)phenyl) propanoate A Q4 (100 mg, 0.172 mmol) in THE (2.00 mL), H₂O (2.00 mL) and LiOH H₂O (40.0 mg, 0.858 mmol) were added and the mixture stirred at 25° C. for 12 hrs. The reaction mixture was filtered, and the filtrate was concentrated to dryness. The residue was purified by prep-HPLC (Column: Xtimate C18 150*40 mm*10 μm; mobile phase: [water (NH₄HCO₃)-ACN]; B %: 6%-46%, 2 min) to give 2-(3-((4-(2-(2-aminopyridin-3-yl)-5-phenyl-3H-imidazo[4,5-b]pyridin-3-yl)benzyl)carbamoyl)phenyl) propanoic acid 36 (17.0 mg, 0.0294 mmol, 17.1% yield) as a light yellow solid. ¹H NMR (400 MHz, DMSO-d₆) δ=9.16 (t, J=6.0 Hz, 1H), 8.26 (d, J=8.0 Hz, 1H), 8.06-7.95 (m, 4H), 7.88-7.77 (m, 2H), 7.50-7.36 (m, 9H), 7.22 (dd, J=1.6, 7.6 Hz, 1H), 6.95 (s, 2H), 6.42 (dd, J=4.8, 7.6 Hz, 1H), 4.61 (d, J=6.0 Hz, 2H), 3.72 (q, J=7.2 Hz, 1H), 1.39 (d, J=7.2 Hz, 3H). HPLC Rₜ=2.064 min in 8 min chromatography, purity 98.2%. LCMS Rₜ=0.620 min in 2.5 min chromatography, purity 97.2%, MS ESI calcd, for 568.22 [M+H]⁺ 569.22, found 569.3.

### Example 37. Synthesis of 2-(3-((4-(2-(2-aminopyridin-3-yl)-5-phenyl-3H-imidazo[4,5-b]pyridin-3-yl)benzyl)carbamoyl)-2-fluorophenyl) propanoic acid (Compound 37)

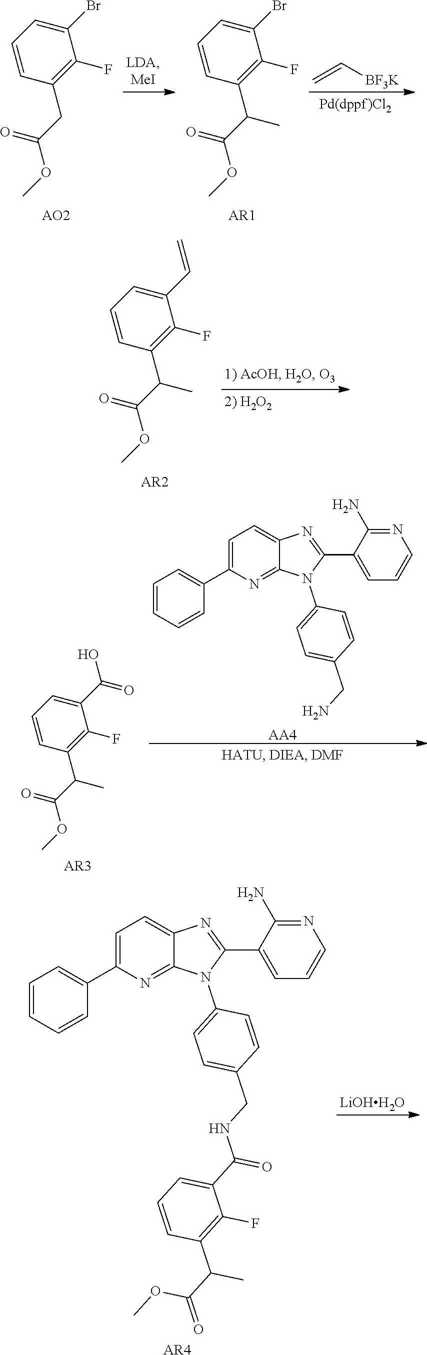

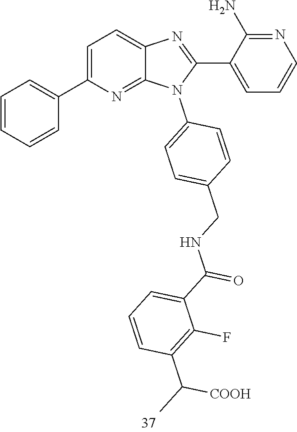 [SMILES: CC(C(=O)O)c1cccc(C(=O)NCc2ccc(-n3c(-c4cccnc4N)nc4ccc(-c5ccccc5)nc43)cc2)c1F]

Step 1. To a solution of methyl 2-(3-bromo-2-fluorophenyl)acetate AO2 (1.00 g, 4.05 mmol) in THF (15.0 mL), LDA (520 mg, 4.86 mmol, 2 M) was added dropwise at −78° C. and the mixture was stirred at −78° C. for 0.5 hr. A solution of iodomethane (1.15 g, 8.10 mmol) in THF (2.00 mL) was added into the mixture at −78° C. and the mixture was stirred at −78° C. for 1 hr, and then subsequently stirred at 25° C. for 16 hrs. The reaction was quenched with sat aq. NH₄Cl (5 mL), drop-wise, and the resulting mixture was extracted with EtOAc (10 mL×3). The combined organic phase was washed with brine (10 mL), dried over anhydrous Na₂SO₄, filtered and the filtrate was concentrated. The residue was purified by flash silica gel chromatography (eluent with 0-5% of EtOAc in PE) to give methyl 2-(3-bromo-2-fluorophenyl) propanoate AR1 (870 mg, 3.33 mmol, 82.3% yield) as a yellow oil. ¹H NMR (400 MHz, DMSO-d₆) δ_{H}=7.65-7.58 (m, 1H), 7.39-7.33 (m, 1H), 7.19-7.12 (m, 1H), 4.06 (q, J=7.2 Hz, 1H), 3.61 (s, 3H), 1.41 (d, J=7.2 Hz, 3H).

Step 2. To a solution of methyl 2-(3-bromo-2-fluorophenyl) propanoate AR1 (870 mg, 3.33 mmol) and potassium vinyltrifluoroborate (580 mg, 4.33 mmol) in 1,4-dioxane (20.0 mL) and water (3.00 mL), Cs₂CO₃ (3.26 g, 10.0 mmol) and Pd(dppf)Cl₂ (242 mg, 0.333 mmol) were added and the mixture stirred at 100° C. for 12 hrs. The reaction mixture was concentrated, water (5 mL) was added to the residue, and the resulting mixture was extracted with EtOAc (10 mL×3). The combined organic phase was washed with brine (10 mL), dried over anhydrous Na₂SO₄, filtered and concentrated. The residue was purified by flash silica gel chromatography (eluent with 0-10% of EtOAc in PE) to give methyl 2-(2-fluoro-3-vinylphenyl) propanoate AR2 (550 mg, 2.64 mmol, 79.3% yield) as a yellow oil. ¹H NMR (400 MHz, DMSO-d₆) δ_{H}=7.60-7.52 (m, 1H), 7.29-7.13 (m, 2H), 6.84 (dd, J=11.2, 17.6 Hz, 1H), 5.95-5.87 (m, 1H), 5.47-5.39 (m, 1H), 4.02 (q, J=7.2 Hz, 1H), 3.60 (s, 3H), 1.41 (d, J=7.2 Hz, 3H).

Step 3. A solution of methyl 2-(2-fluoro-3-vinylphenyl) propanoate AR2 (500 mg, 2.40 mmol) in acetic acid (6.00 mL) and water (6.00 mL) was stirred at 0° C. under O₃ for 0.5 hr. H₂O₂ (0.710 mL, 29.4 mmol) was then added into the mixture under N₂, and the mixture was stirred at 50° C. for 2 hrs. Sat. aq. Na₂SO₃ (5 mL) was added, and the resulting mixture was extracted with EtOAc (5 mL×3). The combined organic phase was washed with brine (5 mL), dried over anhydrous Na₂SO₄, filtered and concentrated. The residue was purified by prep-HPLC (Column: Welch Xtimate C18 150*30 mm*5 μm; mobile phase: [water (HCl)-ACN]; 8%: 36%-44%; 36 min) to give 2-fluoro-3-(1-methoxy-1-oxopropan-2-yl)benzoic acid AR3 (110 mg, 0.486 mmol, 20.3% yield) as a white solid. ¹H NMR (400 MHz, DMSO-d₆) δ_{H}=13.72 (s, 1H), 7.79-7.72 (m, 1H), 7.60-7.53 (m, 1H), 7.27 (t, J=7.6 Hz, 1H), 4.07 (q, J=7.2 Hz, 1H), 3.61 (s, 3H), 1.42 (d, J=7.2 Hz, 3H).

Step 4. To a solution of 3-(3-(4-(aminomethyl)phenyl)-5-phenyl-3H-imidazo[4,5-b]pyridin-2-yl)pyridin-2-amine AA4 (200 mg, 0.510 mmol) and 2-fluoro-3-(1-methoxy-1-oxopropan-2-yl)benzoic acid AR3 (110 mg, 0.510 mmol) in DMF (5.00 mL), DIEA (198 mg, 1.53 mmol) and HATU (233 mg, 0.612 mmol) were added and the mixture was stirred at 25° C. for 4 hrs. Water (5 mL) was added to the reaction mixture and the resulting mixture was extracted with EtOAc (10 mL×3). The combined organic phase was washed with brine (10 mL), dried over anhydrous Na₂SO₄, filtered and concentrated. The residue was purified by flash silica gel chromatography (eluent with 0-5% of MeOH in DCM) to afford methyl 2-(3-((4-(2-(2-aminopyridin-3-yl)-5-phenyl-3H-imidazo[4,5-b]pyridin-3-yl)benzyl)carbamoyl)-2-fluorophenyl) propanoate AR4 (270 mg, 0.450 mmol, 88.2% yield) as a yellow oil. ¹H NMR (400 MHz, DMSO-d₆) δ_{H}=9.03 (t, J=6.0 Hz, 1H), 8.27 (d, J=8.4 Hz, 1H), 8.05-7.98 (m, 4H), 7.50-7.44 (m, 7H), 7.42-7.38 (m, 1H), 7.31-7.22 (m, 3H), 6.99 (s, 2H), 6.43 (dd, J=4.8, 7.6 Hz, 1H), 4.58 (d, J=6.0 Hz, 2H), 4.10-4.06 (m, 1H), 3.61 (s, 3H), 1.43 (d, J=7.2 Hz, 3H).

Step 5. To a solution of methyl 2-(3-((4-(2-(2-aminopyridin-3-yl)-5-phenyl-3H-imidazo[4,5-b]pyridin-3-yl)benzyl)carbamoyl)-2-fluorophenyl) propanoate AR4 (210 mg, 0.350 mmol) in THE (4.00 mL) and water (4.00 mL), LiOH·H₂O (73.0 mg, 1.75 mmol) was added and the mixture stirred at 25° C. for 4 hrs. The reaction mixture was concentrated directly and the residue was purified by prep-HPLC (Column: Welch Xtimate C18 150*30 mm*5 μm; mobile phase: [water (HC)-ACN], B %: 36%-44%; 36 min) to afford 2-(3-(4-(2-(2-aminopyridin-3-yl)-5-phenyl-3H-imidazo[4,5-b]pyridin-3-yl)benzyl)carbamoyl)-2-fluorophenyl) propanoic acid 37 (99.6 mg, 0.169 mmol, 48.4% yield) as a light yellow solid. ¹H NMR (400 MHz, DMSO-d₆) δH=9.02 (t, J=5.6 Hz, 1H), 8.27 (d, J=8.4 Hz, 1H), 8.05-7.97 (m, 4H), 7.57-7.44 (m, 7H), 7.42-7.36 (m, 1H), 7.28-7.20 (m, 2H), 6.97 (s, 2H), 6.42 (dd, J=4.8, 7.6 Hz, 1H), 4.58 (d, J=6.0 Hz, 2H), 3.94 (q, J=7.2 Hz, 1H), 1.40 (d, J=7.2 Hz, 3H). HPLC Rₜ=2.778 min in 8 min chromatography, purity 99.6%. LCMS Rₜ=1.225 min in 2 min chromatography, purity 99.4%, MS ESI calcd, for 586.21, [M+H]⁺ 587.21, found 587.0.

### Example 38. Synthesis of 2-(6-((4-(2-(2-aminopyridin-3-yl)-5-phenyl-3H-imidazo[4,5-b]pyridin-3-yl)benzyl)carbamoyl)pyridin-2-yl) propanoic acid (Compound 38)

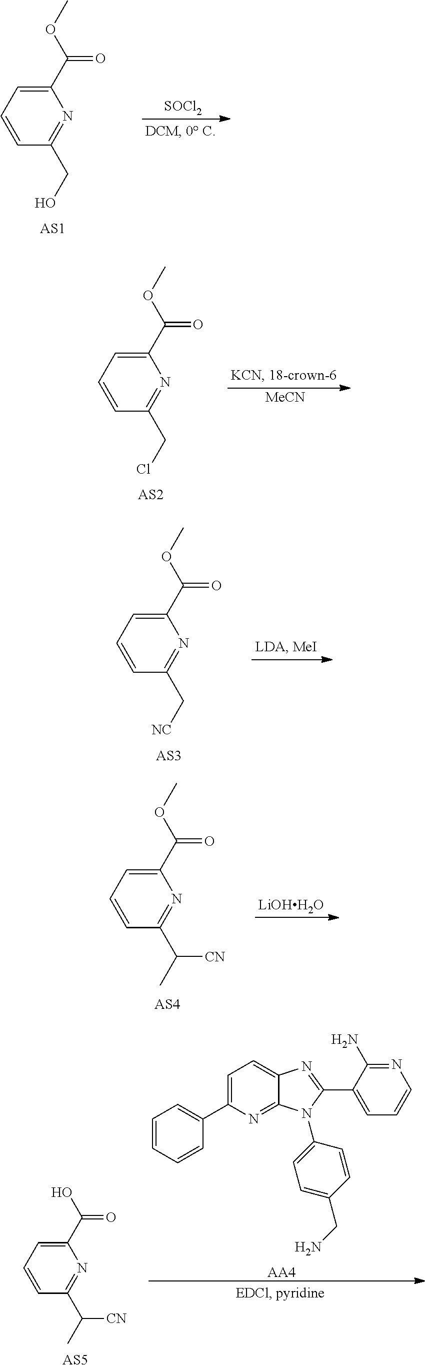

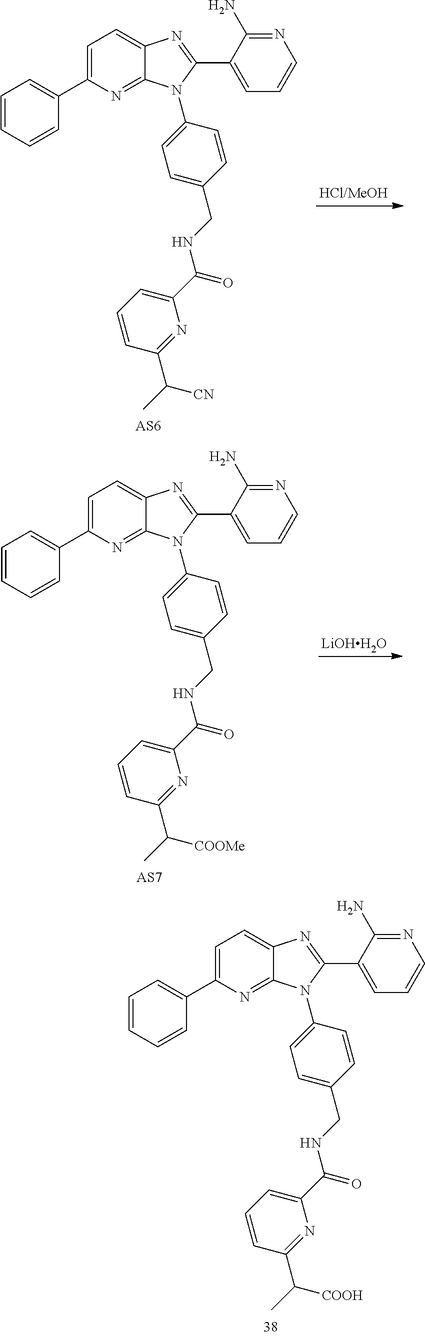 [SMILES: CC(C#N)c1cccc(C(=O)NCc2ccc(-n3c(-c4cccnc4N)nc4ccc(-c5ccccc5)nc43)cc2)n1 ; COC(=O)C(C)c1cccc(C(=O)NCc2ccc(-n3c(-c4cccnc4N)nc4ccc(-c5ccccc5)nc43)cc2)n1 ; "LiOH·H2O" ; CC(C(=O)O)c1cccc(C(=O)NCc2ccc(-n3c(-c4cccnc4N)nc4ccc(-c5ccccc5)nc43)cc2)n1]

Step 1. To a solution of methyl 6-(hydroxymethyl)pyridine-2-carboxylate AS1 (5.00 g, 29.9 mmol) in DCM (50.0 mL), thionyl chloride (11.0 mL, 150 mmol) was added dropwise at 0° C. and the mixture was stirred at 0° C. for 1 hr. The reaction mixture was concentrated, aq. NaHCO₃ (200 mL) was added to the residue, and the resulting mixture was extracted with DCM (100 mL×3). The combined organic phase was washed with brine (50 mL), water (50 mL), dried over anhydrous Na₂SO₄, filtered and concentrated to give methyl 6-(chloromethyl)pyridine-2-carboxylate AS2 (5.00 g, 90.1% yield) as a yellow oil, which was used directly in the next step.

Step 2. To a solution of methyl 6-(chloromethyl)pyridine-2-carboxylate AS2 (4.00 g. 21.6 mmol) in MeCN (50.0 mL), 1,4,7,10,13,16-hexaoxacyclooctadecane (6.27 g, 23.7 mmol) and KCN (1.60 g. 24.6 mmol) were added at 25° C. under a N₂ atmosphere. The suspension was degassed and purged with N₂ (×3), and the mixture was stirred at 80° C. for 4 hrs. Water (200 mL) was added, and the resulting mixture was extracted with DCM (200 mL×3). The combined organic phase was washed with brine (50 mL), water (50 mL), dried over anhydrous Na₂SO₄, filtered and concentrated. The residue was purified by flash silica gel chromatography (eluent with 0-23% of EtOAc in PE) to give methyl 6-(cyanomethyl) picolinate AS3 (1.50 g, 39.5% yield) as a light yellow solid. ¹H NMR (400 MHz, DMSO-d₆) δ=8.09-8.00 (m, 2H), 7.69 (dd, J=7.2, 1.6 Hz, 1H), 4.31 (s, 2H), 3.90 (s, 3H).

Step 3. To a solution of methyl 6-(cyanomethyl) picolinate AS3 (1.50 g, 9.99 mmol) in THF (20.0 mL), LDA (6.00 mL, 12.0 mmol) was added at −78° C., and the mixture was stirred at −78° C. for 0.5 hr. Iodomethane (1.20 mL, 20.0 mmol) was then added in the reaction mixture, dropwise, the reaction was allowed to warm to 25° C., and stirred at 25° C. for 16 hrs. The reaction was quenched with water (10 mL) dropwise, and the resulting mixture was extracted with EtOAc (30 mL×3). The combined organic phase was washed with brine (10 mL), water (10 mL), dried over anhydrous Na₂SO₄, filtered and concentrated. The residue was purified by flash silica gel chromatography (eluent with 0-10% of MeOH in DCM) to give methyl 6-(1-cyanoethyl) picolinate AS4 (500 mg, 3.05 mmol, 30.5% yield) as a brown oil. ¹H NMR (400 MHz, CHLOROFORM-d) 8=8.09 (d, J=7.6 Hz, 1H), 7.92 (t, J=8.0 Hz, 1H), 7.74 (d, J=8.0 Hz, 1H), 4.22 (q, J=7.2 Hz, 1H), 4.01 (s, 3H), 1.74 (d, J=7.2 Hz, 3H).

Step 4. To a solution of methyl 6-(1-cyanoethyl) picolinate AS4 (500 mg, 2.63 mmol) in THF (2.00 mL) and water (2.00 mL), LiOH·H₂O (108 mg, 2.63 mmol) was added at 25° C., and the mixture was stirred at 25° C. for 2 hrs. The residue was purified by prep-HPLC (column: Xtimate C18 150*40 mm*10 μm; condition; water (HCl)-ACN; begin b: 0-28%) to give 6-(1-cyanoethyl) picolinic acid AS5 (270 mg, 88.2% yield) as a white solid. ¹H NMR (400 MHz, DMSO-d₆) δ=7.99-7.89 (m, 2H), 7.66-7.60 (m, 1H), 3.86-3.84 (m, 1H), 1.41 (d, J=7.2 Hz, 3H).

Step 5, To a solution of 3-(3-(4-(aminomethyl)phenyl)-5-phenyl-3H-imidazo[4,5-b]pyridin-2-yl)pyridin-2-amine AA4 (250 mg, 0.637 mmol) in pyridine (6.00 mL), 6-(1-cyanoethyl) picolinic acid AS5 (112 mg, 0.637 mmol) and EDCI (182 mg, 0.955 mmol) were added at 25° C., and the mixture stirred for 16 hrs. The reaction mixture was concentrated directly, and the residue was purified by flash silica gel chromatography (eluent with 0-10% of MeOH in DCM) to give N-(4-(2-(2-aminopyridin-3-yl)-5-phenyl-3H-imidazo[4,5-b]pyridin-3-yl)benzyl)-6-(1-cyanoethyl) picolinamide AS6 (160 mg, 45.6% yield) as a brown oil. LCMS Rₜ=0.602 min in 1.0 min chromatography, purity 62.9%, MS ESI calcd, for 550.22, [M+H]⁺ 551.22, found 551.3.

Step 6. A solution of N-(4-(2-(2-aminopyridin-3-yl)-5-phenyl-3H-imidazo[4,5-b]pyridin-3-yl)benzyl)-6-(1-cyanoethyl) picolinamide AS6 (80.0 mg, 0.145 mmol) in methanol (3.00 mL), under N₂ atmosphere, was degassed and purged with N₂ (×3). The reaction mixture was then purged with HCl (gas) and stirred at −78° C. for 0.5 hr. After addition, the mixture was concentrated to remove MeOH, water (3.00 mL) was added, and the reaction mixture was stirred at 25° C. for 0.5 hr. The resulting mixture was extracted with DCM (10 mL×3), and the combined organic phase was washed with brine (5 mL), water (5 mL), dried over anhydrous Na₂SO₄, filtered and concentrated to give methyl 2-(6-((4-(2-(2-aminopyridin-3-yl)-5-phenyl-3H-imidazo[4,5-b]pyridin-3-yl)benzyl)carbamoyl)pyridin-2-yl) propanoate AS7 (50.0 mg, 59.0% yield) as a yellow solid. ¹H NMR (400 MHz, DMSO-d₆) δ=9.30-9.17 (m, 1H), 8.35 (d, J=8.4 Hz, 2H), 8.10 (d, J=6.0 Hz, 1H), 8.05 (dd, J=8.0, 3.6 Hz, 3H), 8.01-7.95 (m, 2H), 7.87 (br d, J=7.6 Hz, 1H), 7.61-7.52 (m, 3H), 7.52-7.45 (m, 4H), 7.44-7.39 (m, 1H), 6.91-6.85 (m, 1H), 4.64 (d, J=6.4 Hz, 2H), 4.11 (q, J=7.2 Hz, 1H), 3.61 (s, 3H), 1.55-1.49 (m, 3H).

Step 7. To a solution of methyl 2-(6-((4-(2-(2-aminopyridin-3-yl)-5-phenyl-3H-imidazo[4,5-b]pyridin-3-yl)benzyl)carbamoyl)pyridin-2-yl) propanoate AS7 (50.0 mg, 0.0857 mmol) in THE (1.00 mL) and water (1.00 mL), LiOH·H₂O) (5.30 mg, 0.129 mmol) was added at 25° C., and the mixture stirred for 2 hrs. The residue was purified by prep-HPLC (column: Xtimate C18 150*40 mm*10 μm; condition: water (HCl)-ACN; begin B: 10-50) to give 2-(6-((4-(2-(2-aminopyridin-3-yl)-5-phenyl-3H-imidazo[4,5-b]pyridin-3-yl)benzyl)carbamoyl)pyridin-2-yl) propanoic acid 38 (17.0 mg, 34.0% yield) as a yellow solid. ¹H NMR (400 MHz, DMSO-d₆) δ=9.26 (t, J=6.4 Hz, 1H), 8.34 (d, J=8.4 Hz, 2H), 8.09 (d, J=6.4 Hz, 1H), 8.05 (dd, J=8.0, 3.6 Hz, 3H), 8.01-7.94 (m, 2H), 7.90-7.84 (m, 1H), 7.59 (dd, J=6.8, 2.4 Hz, 1H), 7.56-7.45 (m, 6H), 7.44-7.38 (m, 1H), 6.91-6.84 (m, 1H), 4.64 (d, J=6.4 Hz, 2H), 3.98 (q, J=7.2 Hz, 1H), 1.51 (d, J=7.2 Hz, 3H). HPLC Rₜ=2.772 min in 8 min chromatography, purity 100%. LCMS Rₜ=1.225 min in 2.0 min chromatography, purity 98.8%, MS ESI calcd, for 569.22, [M+H]⁺ 570.22, found 570.3.

### Example 39. Synthesis of methyl 3-(4-((4-(2-(2-aminopyridin-3-yl)-5-phenyl-3H-imidazo [4,5-b]pyridin-3-yl)benzyl)carbamoyl)phenyl) propanoate (Compound 39)

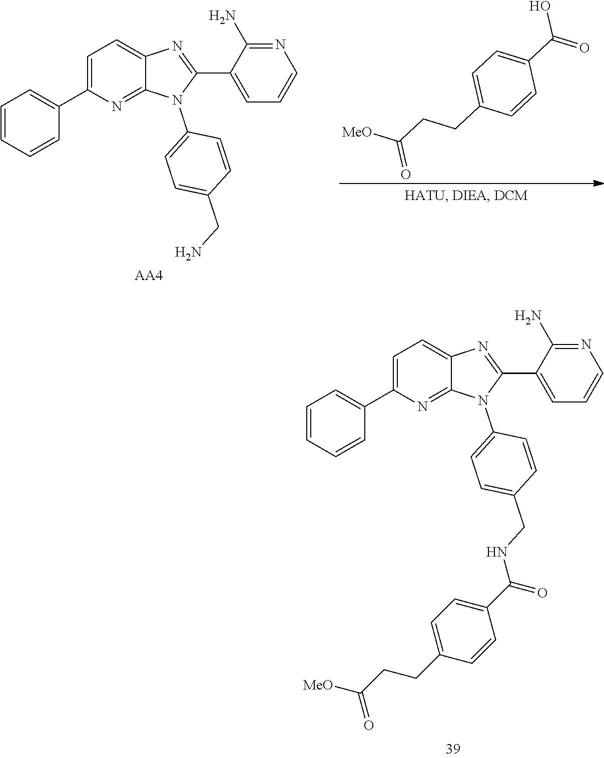

Step 1. To a solution of 3-(3-(4-(aminomethyl)phenyl)-5-phenyl-3H-imidazo[4,5-b]pyridin-2-yl)pyridin-2-amine AA4 (200 mg, 0.510 mmol) in DCM (6.00 mL). 4-(3-methoxy-3-oxo-propyl)benzoic acid (212 mg, 1.02 mmol). DIEA (0.280 mL, 1.53 mmol) and HATU (291 mg, 0.764 mmol) were added at 25° C., and the mixture was stirred for 3 hrs. The reaction mixture was concentrated directly, and the residue was purified by prep-HPLC (Column: Welch Xtimate C18 150*30 mm*5 μm; Condition: water (HCl)-ACN; Begin B: 16-56%) to afford methyl 3-(4-((4-(2-(2-aminopyridin-3-yl)-5-phenyl-3H-imidazo[4,5-b]pyridin-3-yl)benzyl)carbamoyl)phenyl) propanoate 39 (13.0 mg, 0.0226 mmol, 4.43% yield) as a yellow solid. ¹H NMR (400 MHz, DMSO-d₆) δ=9.10 (t, J=6.0 Hz, 1H), 8.34 (d, J=8.4 Hz, 1H), 8.26-8.00 (m, 5H), 7.89-7.77 (m, 3H), 7.58-7.30 (m, 9H), 6.90-6.76 (m, 1H), 4.59 (d, J=6.0 Hz, 2H), 3.58 (s, 3H), 2.95-2.88 (m, 2H), 2.69-2.65 (m, 2H). HPLC Rₜ=2.943 min in 8 min chromatography, purity 99.0%. LCMS Rₜ=1.387 min in 2.5 min chromatography, purity 97.3%, MS ESI calcd, for 582.24 [M+H]⁺ 583.24, found 583.3.

### Example 40. Synthesis of 3-(4-((4-(2-(2-aminopyridin-3-yl)-5-phenyl-3H-imidazo[4,5-b]pyridin-3-yl)benzyl)carbamoyl)phenyl) propanoic acid (Compound 40)

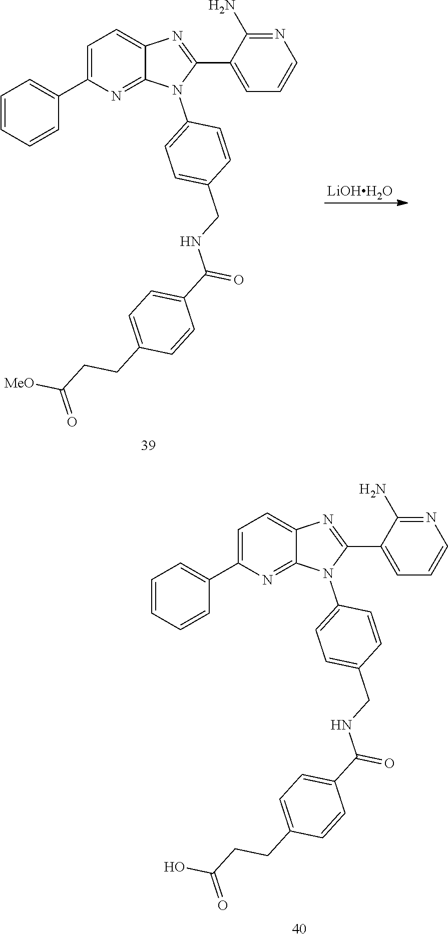 [SMILES: COC(=O)CCc1ccc(C(=O)NCc2ccc(-n3c(-c4cccnc4N)nc4ccc(-c5ccccc5)nc43)cc2)cc1 ; "LiOH·H2O" ; Nc1ncccc1-c1nc2ccc(-c3ccccc3)nc2n1-c1ccc(CNC(=O)c2ccc(CCC(=O)O)cc2)cc1]

Step 1. To a solution of methyl 3-(4-((4-(2-(2-aminopyridin-3-yl)-5-phenyl-3H-imidazo[4,5-b]pyridin-3-yl)benzyl)carbamoyl)phenyl) propanoate 39 (200 mg, 0.343 mmol) in THF (3.00 mL) and water (3.00 mL), LiOH·H₂O (43.0 mg, 1.03 mmol) was added at 25° C., and the mixture was stirred for 2 hrs. The reaction mixture was concentrated to remove THE, and the residue was purified by prep-HPLC (Column: Welch Xtimate C18 150*30 mm*5 μm, Condition: water (HCl)-ACN; Begin B: 10-50%) to afford 3-(4-((4-(2-(2-aminopyridin-3-yl)-5-phenyl-3H-imidazo[4,5-b]pyridin-3- yl)benzyl)carbamoyl)phenyl) propanoic acid 40 (18.0 mg, 0.0284 mmol, 8.26% yield) as a yellow solid. ¹H NMR (400 MHz, DMSO-d₆) δ=9.12 (t. J=6.0 Hz, 1H), 8.62-8.21 (m, 3H), 8.15-7.99 (m, 4H), 7.95-7.80 (m, 3H), 7.59-7.31 (m, 9H), 6.89 (t, J=6.8 Hz, 1H), 4.59 (d, J=6.0 Hz, 2H), 2.88 (t, J=7.2 Hz, 2H), 2.57 (t, J=7.6 Hz, 2H). HPLC Rₜ=3.750 min in 8 min chromatography, purity 90.6%. LCMS Rₜ=1.289 min in 2.5 min chromatography, purity 92.5%, MS ESI calcd, for 568.22 [M+H]⁺ 569.22. found 569.3.

### Example 41. Synthesis of methyl 3-(3-((4-(2-(2-aminopyridin-3-yl)-5-phenyl-3H-imidazo[4,5-b]pyridin-3-yl)benzyl)carbamoyl)phenyl) propanoate (Compound 41)

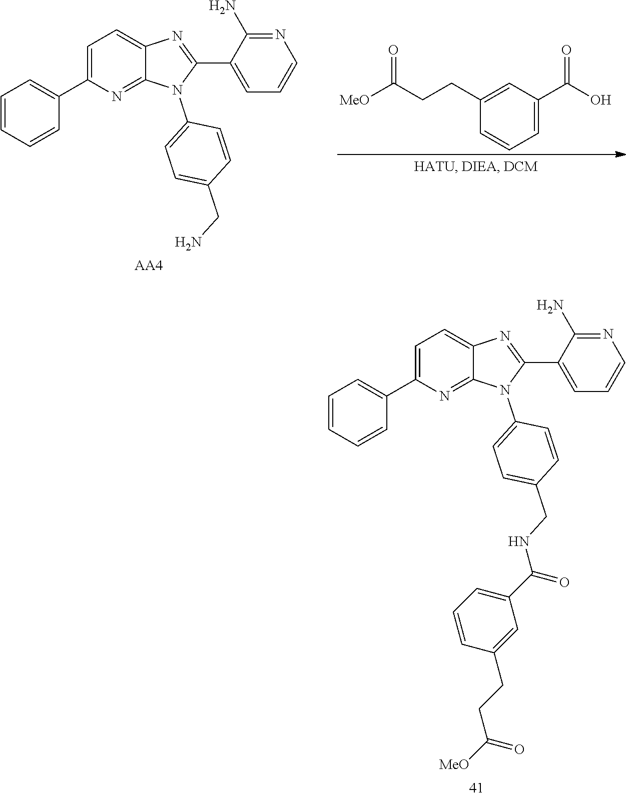

Step 1. To a solution of 3-(3-(4-(aminomethyl)phenyl)-5-phenyl-3H-imidazo[4,5-b]pyridin-2-yl)pyridin-2-amine AA4 (400 mg, 1.02 mmol) in DCM (6.00 mL), 3-(3-methoxy-3-oxo-propyl)benzoic acid (318 mg, 1.53 mmol), DIEA (395 mg, 3.06 mmol) and HATU (581 mg. 1.53 mmol) were added at 25° C., and the mixture was stirred for 3 hrs. The reaction mixture was concentrated directly, and the resulting residue was purified by prep-HPLC (Column: Welch Xtimate C18 150*30 mm*5 μm; Condition: water (HCl)-ACN; Begin B: 16-56%) to give methyl 3-(3-((4-(2-(2-aminopyridin-3-yl)-5-phenyl-3H-imidazo[4,5-b]pyridin-3-yl)benzyl)carbamoyl)phenyl) propanoate 41 (17.0 mg, 0.0292 mmol, 2.87% yield) as a yellow solid. ¹H NMR (400 MHz, DMSO-d₆) δ=9.15 (t, J=6.0 Hz, 1H), 8.54-8.32 (m, 2H), 8.14-8.01 (m, 4H), 7.91 (dd, J=7.6, 1.6 Hz, 1H), 7.84-7.73 (m, 2H), 7.59-7.36 (m, 9H), 6.90 (dd, J=7.2, 6.4 Hz, 1H), 4.60 (d, J=6.0 Hz, 2H), 3.58 (s, 3H), 2.94-2.88 (m, 2H), 2.68 (t, J=7.6 Hz, 2H). HPLC Rₜ=4.161 min in 8 min chromatography, purity 99.6%. LCMS Rₜ=1.305 min in 2 min chromatography, purity 98.2%, MS ESI calcd, for 582.24 [M+H]⁺ 583.24, found 583.3.

### Example 42. Synthesis of 3-(3-((4-(2-(2-aminopyridin-3-yl)-5-phenyl-3H-imidazo[4,5-b]pyridin-3-yl)benzyl)carbamoyl)phenyl) propanoic acid (Compound 42)

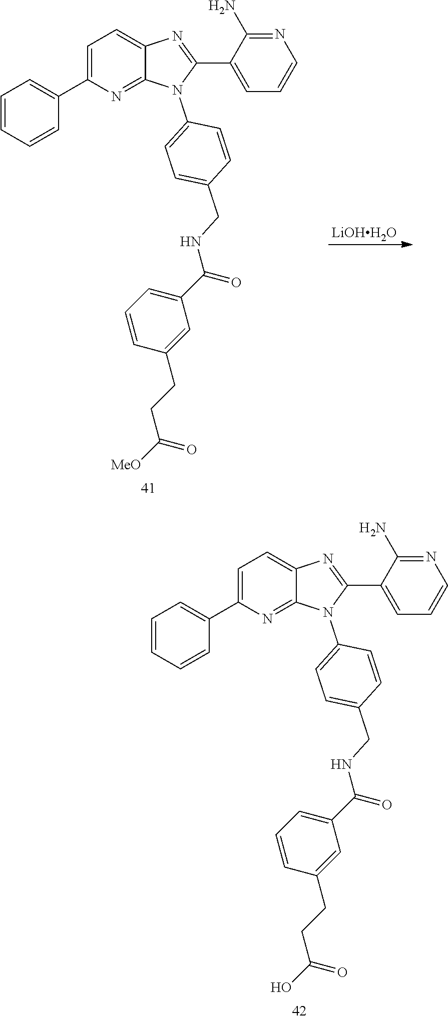 [SMILES: COC(=O)CCc1cccc(C(=O)NCc2ccc(-n3c(-c4cccnc4N)nc4ccc(-c5ccccc5)nc43)cc2)c1 ; "LiOH·H2O" ; Nc1ncccc1-c1nc2ccc(-c3ccccc3)nc2n1-c1ccc(CNC(=O)c2cccc(CCC(=O)O)c2)cc1]

Step 1. To a solution of methyl 3-(3-((4-(2-(2-aminopyridin-3-yl)-5-phenyl-3H-imidazo[4,5-b]pyridin-3-yl)benzyl)carbamoyl)phenyl) propanoate 41 (100 mg, 0.172 mmol) in THF (3.00 mL) and water (3.00 mL), LiOH·H₂O (22.00 mg, 0.515 mmol) was added at 25° C., and the mixture was stirred for 2 hrs. The reaction mixture was concentrated to remove THE, and the resulting residue was purified by prep-HPLC (Column: Welch Xtimate C18 150*30 mm*5 μm; Condition: water (HCl)-ACN; Begin B: 12-52%) to give 3-(3-((4-(2-(2-aminopyridin-3-yl)-5-phenyl-3H-imidazo[4,5-b]pyridin-3-yl)benzyl)carbamoyl)phenyl) propanoic acid 42 (12.0 mg, 0.0206 mmol, 11.9% yield) as a yellow solid. ¹H NMR (400 MHz, DMSO-d₆) δ=9.15 (t, J=6.0 Hz, 1H), 8.53-8.33 (m, 2H), 8.12 (dd, J=1.6, 6.4 Hz, 1H), 8.08-8.02 (m, 3H), 7.90 (dd, J=7.6, 1.6 Hz, 1H), 7.81 (s, 1H), 7.76 (dt, J=6.8, 1.6 Hz, 1H), 7.58-7.45 (m, 6H), 7.44-7.37 (m, 3H), 6.90 (dd, J=7.2, 6.4 Hz. 1H), 4.60 (d, J=6.0 Hz, 2H), 2.88 (t, J=7.6 Hz, 2H), 2.59 (t, J=7.6 Hz, 2H). HPLC Rₜ=2.626 min in 8 min chromatography, purity 100%. LCMS Rₜ=1.206 min in 2 min chromatography, purity 98.2%, MS ESI calcd, for 568.22 [M+H]⁺ 569.22, found 569.3.

### Example 43. Synthesis of 3-(2-amino-2-oxoethyl)-N-(4-(2-(2-aminopyridin-3-yl)-5-phenyl-3H-imidazo[4,5-b]pyridin-3-yl)benzyl)benzamide (Compound 43)

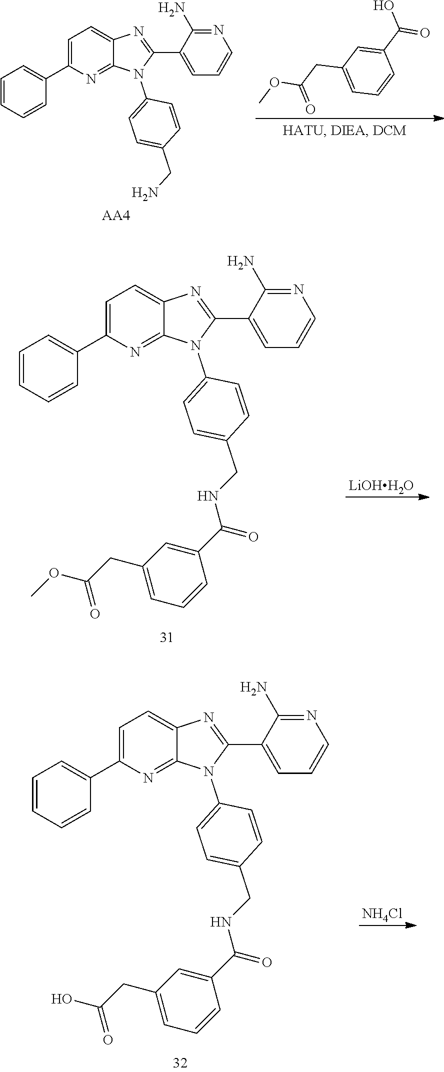

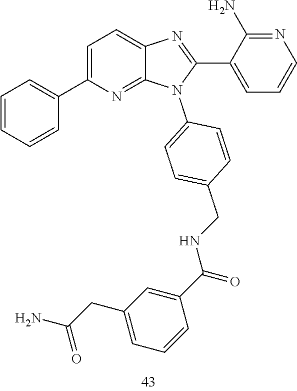 [SMILES: NC(=O)Cc1cccc(C(=O)NCc2ccc(-n3c(-c4cccnc4N)nc4ccc(-c5ccccc5)nc43)cc2)c1]

Step 1. To a mixture of 3-(3-(4-(aminomethyl)phenyl)-5-phenyl-3H-imidazo[4,5-b]pyridin-2-yl)pyridin-2-amine AA4 (150 mg, 0.380 mmol) in DCM (3.00 mL), 3-(2-methoxy-2-oxo-ethyl)benzoic acid (82.0 mg, 0.420 mmol) and DIEA (148 mg, 1.15 mmol) were added. HATU (199 mg, 0.520 mmol) was then added to the mixture, and the mixture was stirred at 25° C. for 2 hrs. The reaction mixture was poured into water (20 mL), and the resulting mixture was extracted with EtOAc (20 ml×3). The combined organic phase was washed with brine (30 mL×3), dried over anhydrous Na₂SO₄, filtered and concentrated. The crude product was purified by combi-flash column (EtOAc in PE=55%) to give methyl 2-(3-((4-(2-(2-aminopyridin-3-yl)-5-phenyl-3H-imidazo[4,5-b]pyridin-3-yl)benzyl)carbamoyl)phenyl)acetate 31 (232 mg, crude) as a yellow solid. ¹H NMR (400 MHz, DMSO-d₆) δ_{H}=9.15 (t, J=6.0 Hz, 1H), 8.26 (d, J=8.4 Hz, 1H), 8.22-8.12 (m, 1H), 8.06-7.95 (m, 4H), 7.85-7.80 (m, 2H), 7.50-7.42 (m, 8H), 7.22 (dd, J=1.6, 7.6 Hz, 1H), 6.95 (s, 2H), 6.42 (dd, J=4.8, 7.6 Hz, 1H), 4.60 (d, J=6.0 Hz, 2H), 3.76 (s, 2H), 3.62 (s, 3H).

Step 2. To a solution of methyl 2-(3-((4-(2-(2-aminopyridin-3-yl)-5-phenyl-3H-imidazo[4,5-b]pyridin-3-yl)benzyl)carbamoyl)phenyl)acetate 31 (200 mg, 0.350 mmol) in THF (5.00 mL) and water (2.00 mL), LiOH·H₂O (74.0 mg, 1.76 mmol) was added and the mixture was stirred at 25° C. for 12 hrs. The reaction mixture was poured into water (15 mL), and the resulting mixture was extracted with PE (30 mL). The water phase was adjusted to pH 4 by aqueous HCl (1 M) and subsequently extracted with EtOAc (30 ml×3). The combined organic phase was dried over Na₂SO₄, filtered and concentrated to give 2-(3-((4-(2-(2-aminopyridin-3-yl)-5-phenyl-3H-imidazo[4,5-b]pyridin-3-yl)benzyl)carbamoyl)phenyl)acetic acid 32 (150 mg, 76.9% yield) as a yellow solid.

Step 3. To a solution of 2-(3-((4-(2-(2-aminopyridin-3-yl)-5-phenyl-3H-imidazo[4,5-b]pyridin-3-yl)benzyl)carbamoyl)phenyl)acetic acid 32 (150 mg, 0.270 mmol) in DMF (3.00 mL), DIEA (105 mg, 0.810 mmol) and NH₄Cl (17.0 mg, 0.320 mmol) were added. HATU (134 mg, 0.350 mmol) was then added to the mixture, and the mixture was stirred at 25° C. for 2 hrs. The reaction mixture was diluted with DMF (4.00 mL), and purified by prep-HPLC (column: welch xtimate c18 150*40 mm*10 μm; mobile phase: [water (NH₄H₂O+NH₄HCO₃)-ACN]; B %: 18%-58%: 36 min) to give 3-(2-amino-2-oxoethyl)-N-(4-(2-(2-aminopyridin-3-yl)-5-phenyl-3H-imidazo[4,5-b]pyridin-3-yl)benzyl)benzamide 43 (11.1 mg, 7.3% yield) as a white solid. ¹H NMR (400 MHz, DMSO-d₆) δ_{H}=9.14 (t, J=6.0 Hz, 1H), 8.26 (d, J=8.4 Hz, 1H), 8.07-7.96 (m, 4H), 7.84-7.76 (m, 2H), 7.56-7.34 (m, 10H), 7.21 (dd, J=2.0, 7.6 Hz, 1H), 6.98-6.88 (m, 3H), 6.42 (dd, J=5.2, 7.6 Hz, 1H), 4.60 (d, J=6.0 Hz, 2H), 3.44 (s, 2H). HPLC R (=2.220 min in 8 min chromatography, purity 99.7%. LCMS Rₜ=1.916 min in 4 min; Agilent PoroShell 120 EC-C18 2.7 μm 3.0*50 mm; purity 99.6%, MS ESI calcd, for 553.22, [M+H]⁺ 554.22, found 554.3.

### Example 44. Synthesis of N-(4-(2-(2-aminopyridin-3-yl)-5-phenyl-3H-imidazo[4,5-b]pyridin-3-yl)benzyl)-3-(2-(ethylamino)-2-oxoethyl)benzamide (Compound 44)

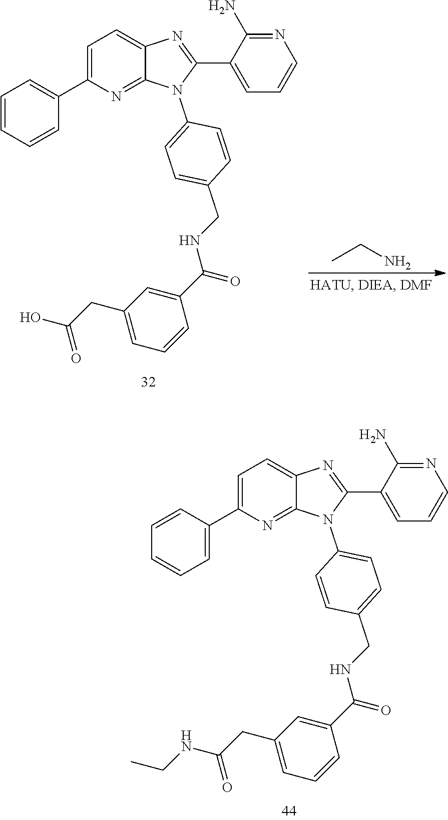

Step 1. To a solution of 2-(3-((4-(2-(2-aminopyridin-3-yl)-5-phenyl-3H-imidazo[4,5-b]pyridin-3-yl)benzyl)carbamoyl)phenyl)acetic acid 32 (160 mg, 0.288 mmol) and ethanamine (65.0 mg, 1.44 mmol) in DMF (5.00 mL), DIEA (112 mg, 0.865 mmol) and HATU (165 mg, 0.433 mmol) were added and the mixture was stirred at 25° C. for 6 hrs. Water (5 mL) was added to the reaction mixture, and the resulting mixture was extracted with EtOAc (5 mL×3). The combined organic phase was washed with brine (5 mL), dried over anhydrous Na₂SO₄, filtered and concentrated. The residue was purified by prep-HPLC (Column: Welch Xtimate C18 150*30 mm*5 μm; mobile phase: [water (NH₃H₂O+NH₄HCO₃)-ACN]; B %: 26%-66%: 20 min) to afford N-(4-(2-(2-aminopyridin-3-yl)-5-phenyl-3H-imidazo[4,5-b]pyridin-3-yl)benzyl)-3-(2-(ethyl amino)-2-oxoethyl)benzamide 44 (4.00 mg, 0.00686 mmol, 2.38% yield) as a light yellow solid. ¹H NMR (DMSO-d₆, 400 MHz) δ_{H}=9.13 (t, J=6.0 Hz, 1H), 8.26 (d, J=8.4 Hz, 1H), 8.08-7.96 (m, 5H), 7.82-7.77 (m, 2H), 7.49-7.36 (m, 9H), 7.24-7.19 (m, 1H), 6.94 (s, 2H), 6.42 (dd, J=4.8, 7.6 Hz, 1H), 4.60 (d, J=6.0 Hz, 2H), 3.45 (s, 2H), 3.11-3.02 (m, 2H), 1.01 (t, J=7.2 Hz, 3H). HPLC Rₜ=1.917 min in 8 min chromatography, purity 99.7%. LCMS Rₜ=1.224 min in 2 min chromatography, purity 98.3%, MS ESI calcd, for 581.25, [M+H]⁺ 582.25, found 582.0.

### Example 45. Synthesis of 4-amino-N-(4-(2-(2-aminopyridin-3-yl)-5-phenyl-3H-imidazo[4,5-b]pyridin-3-yl)benzyl)benzamide (Compound 45)

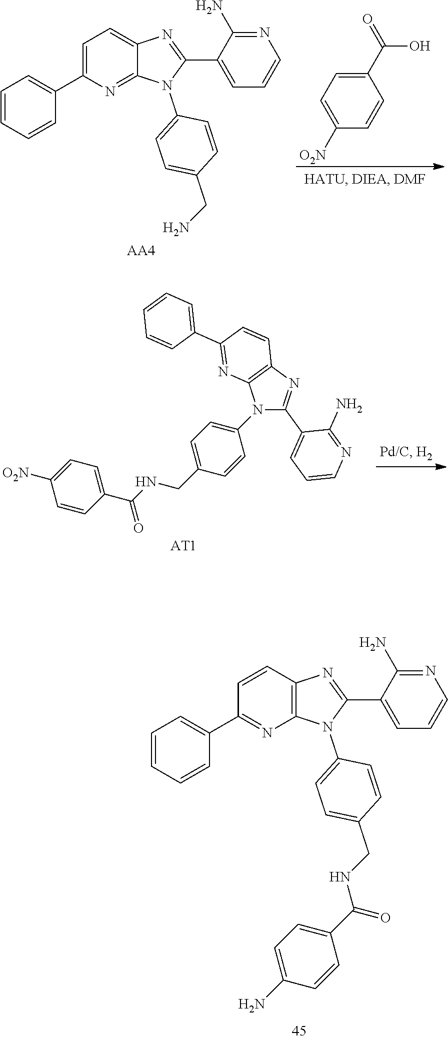

Step 1. To a solution of 3-(3-(4-(aminomethyl)phenyl)-5-phenyl-3H-imidazo[4,5-b]pyridin-2-yl)pyridin-2-amine AA4 (300 mg, 0.764 mmol) and 4-nitrobenzoic acid (128 mg, 0.764 mmol) in DMF (5.00 mL), DIEA (296 mg, 2.29 mmol) and HATU (436 mg, 1.15 mmol) were added and the mixture was stirred at 25° C. for 2 hrs. Water (20 mL) was added to the reaction mixture, and the resulting mixture was extracted with EtOAc (30 mL×3). The combined organic phase was washed with brine (10 mL), dried over anhydrous Na₂SO₄, filtered and the filtrate was concentrated. The residue was purified by flash silica gel chromatography (eluent with 0-80% of EtOAc in PE) to give N-(4-(2-(2-aminopyridin-3-yl)-5-phenyl-3H-imidazo[4,5-b]pyridin-3-yl)benzyl)-4-nitrobenzamide AT1 (380 mg, 0.702 mmol, 91.8% yield) as a yellow oil. ¹H NMR (400 MHz, DMSO-d₆) δ=9.48 (s, 1H), 8.34 (d, J=8.8 Hz, 2H), 8.26 (d, J=8.4 Hz, 1H), 8.16 (d, J=8.8 Hz, 2H), 8.08-7.91 (m, 4H), 7.59-7.30 (m, 7H), 7.22 (dd, J=1.6, 7.6 Hz, 1H), 6.93 (s, 2H), 6.42 (dd, J=4.8, 7.6 Hz, 1H), 4.64 (d, J=6.0 Hz, 2H). HPLC Rₜ=2.832 min in 8 min chromatography, purity 98.5%. LCMS Rₜ=1.840 min in 4 min chromatography, purity 99.7%, MS ESI calcd, for 541.19, [M+H]⁺ 542.19, found 542.5.

Step 2. To a solution of N-(4-(2-(2-aminopyridin-3-yl)-5-phenyl-3H-imidazo[4,5-b]pyridin-3-yl)benzyl)-4-nitrobenzamide AT1 (130 mg, 0.240 mmol) in methanol (3.00 mL), wet Pd/C (20.0 mg, 10% purity) was added under a N₂ atmosphere. The suspension was degassed and purged with H₂ (×3), and the reaction mixture was stirred under H₂ (15 Psi) at 20° C. for 12 hrs. The mixture was filtered, washed with THF (10 mL×3), and concentrated to give 4-amino-N-(4-(2-(2-aminopyridin-3-yl)-5-phenyl-3H-imidazo[4,5-b]pyridin-3-yl)benzyl)benzamide 45 (100 mg, 0.195 mmol, 70.6% yield) as a yellow oil. The crude product (50 mg) was purified by prep-HPLC (column: Welch Xtimate C18 150*30 mm*5 μm; mobile phase: [water (FA)-ACN]; B %: 6%-46%, 25 min) to give 4-amino-N-(4-(2-(2-aminopyridin-3-yl)-5-phenyl-3H-imidazo[4,5-b]pyridin-3-yl)benzyl)benzamide 45 (14.0 mg, 0.0272 mmol, 9.81% yield) as a white solid. ¹H NMR (400 MHz, DMSO-d₆) δ=8.67 (s, 1H), 8.31-8.19 (m, 1H), 8.08-7.92 (m, 4H), 7.65 (d, J=8.8 Hz, 2H), 7.50-7.34 (m, 7H), 7.21 (dd, J=1.6, 7.6 Hz, 1H), 6.95 (s, 2H), 6.56 (d, J=8.8 Hz, 2H), 6.42 (dd, J=4.8, 7.6 Hz, 1H), 5.63 (s, 2H), 4.55 (d, J=6.0 Hz, 2H). HPLC R (=2.203 min in 8 min chromatography, purity 100%. LCMS Rₜ=1.462 min in 4 min chromatography, purity 96.1%, MS ESI calcd, for 511.21, [M+H]⁺ 512.21, found 512.4.

### Example 46. Synthesis of 4-(((4-(2-(2-aminopyridin-3-yl)-5-phenyl-3H-imidazo[4,5-b]pyridin-3-yl)benzyl)amino)methyl)benzoic acid (Compound 46)

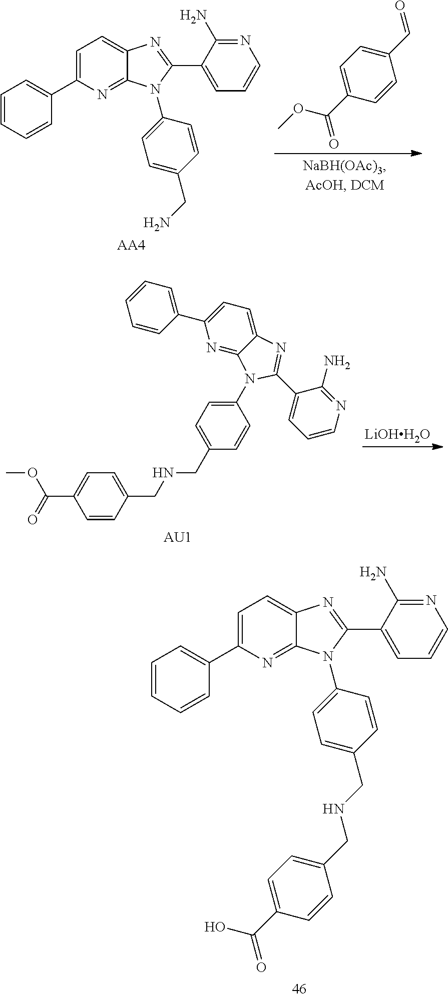

Step 1. To a stirred solution of 3-(3-(4-(aminomethyl)phenyl)-5-phenyl-3H-imidazo[4,5-b]pyridin-2-yl)pyridin-2-amine AA4 (100 mg, 0.250 mmol) and methyl 4-formylbenzoate (63.0 mg, 0.380 mmol) in DCM (2.00 mL), acetic acid (76.0 mg, 1.27 mmol) was added and the mixture was stirred at 25° C. under N₂ for 10 hrs. Sodium triacetoxyborohydride (108 mg, 0.510 mmol) was added to the reaction mixture, and the resulting mixture was stirred at 25° C., under N₂, for 3 hrs. The reaction mixture was diluted with EtOAc (20 mL) and washed with water (20 mL) and brine (20 mL×2), dried over anhydrous sodium sulfate, filtered and the filtrate was concentrated to afford methyl 4-(((4-(2-(2-aminopyridin-3-yl)-5-phenyl-3H-imidazo[4,5-b]pyridin-3-yl)benzyl)amino)methyl)benzoate AU1 (100 mg, 72.6% yield) as a yellow oil. LCMS Rₜ=0.39 min in 1.0 min chromatography, purity 75.8%, MS ESI calcd, for 540.23 [M+H]⁺ 541.23, found 541.4.

Step 2. To a stirred solution of methyl 4-(((4-(2-(2-aminopyridin-3-yl)-5-phenyl-3H-imidazo[4,5-b]pyridin-3-yl)benzyl)amino)methyl)benzoate AU1 (100 mg, 0.180 mmol) in THF (1.00 mL) and water (1.00 mL), LiOH·H₂O (15.0 mg, 0.370 mmol) was added. The reaction mixture was stirred at 25° C., under N₂, for 16 hrs. The reaction mixture was adjusted to a pH ~5 with 1 M HCl, and subsequently concentrated to afford a residue. The crude product was purified by prep-HPLC (column: welch xtimate C18 150×30 mm×5 μm, method: water (FA)-ACN, begin B: 10, end B: 40) to afford 4-(((4-(2-(2-aminopyridin-3-yl)-5-phenyl-3H-imidazo[4,5-b]pyridin-3-yl)benzyl)amino)methyl)benzoic acid 46 (5.00 mg, 0.00820 mmol, 4.44% yield) as a yellow solid. ¹H NMR (400 MHz, DMSO-d₆) δ=8.27 (d, J=8.4 Hz, 1H), 8.09-7.95 (m, 5H), 7.91 (d, J=8.4 Hz, 2H), 7.63-7.33 (m, 9H), 7.20 (dd, J=2.0, 7.6 Hz, 1H), 7.00 (s, 2H), 6.40 (dd, J=4.8, 7.6 Hz, 1H), 3.80 (d, J=8.8 Hz, 4H). HPLC Rₜ=4.172 min in 8 min chromatography, purity 86.5%. LCMS Rₜ=1.595 min in 4.0 min chromatography, purity 94.6%, MS ESI calcd, for 526.21 [M+H]⁺ 527.21, found 527.1.

### Example 47. Synthesis of 2-(3-(((4-(2-(2-aminopyridin-3-yl)-5-phenyl-3H-imidazo[4,5-b]pyridin-3-yl)benzyl)amino)methyl)phenyl)acetic acid (Compound 47)

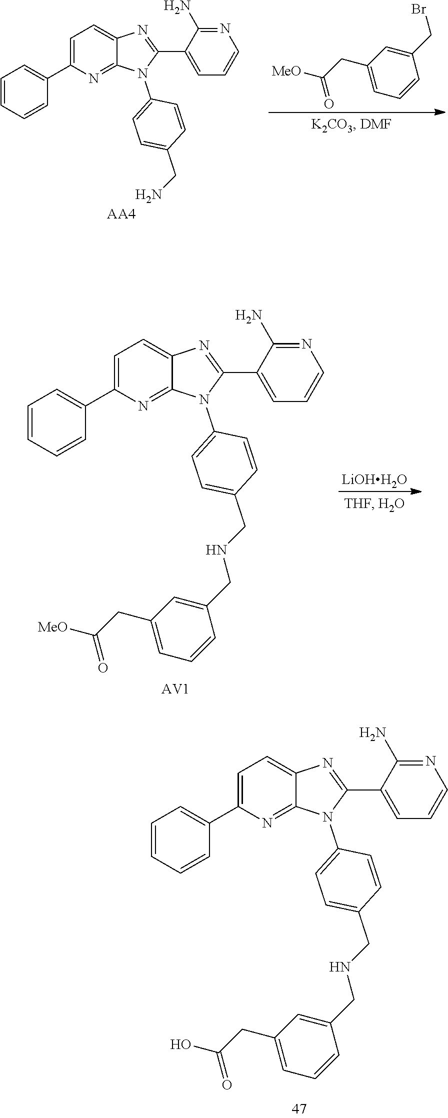

Step 1. To a solution of methyl 2-(3-(bromomethyl)phenyl)acetate (3.50 g, 11.1 mmol) in DMF (40.0 mL), K₂CO₃ (4.60 g, 33.3 mmol) was added under a N₂ atmosphere. Then 3-(3-(4-(aminomethyl)phenyl)-5-phenyl-3H-imidazo[4,5-b]pyridin-2-yl)pyridin-2-amine AA4 (200 mg, 0.510 mmol) was added into the reaction mixture, and the reaction mixture was degassed, purged with N₂ (×3), and stirred at 20° C. for 6 hrs. The reaction was dissolved in EtOAc (100 mL) and washed with water (50 mL) and brine (3×50 mL). The organic layers were then separated and dried over Na₂SO₄, filtered, and concentrated. The crude was purified by flash column chromatography (eluting with 4% of MeOH in DCM) to afford methyl 2-(3-(((4-(2-(2-aminopyridin-3-yl)-5-phenyl-3H-imidazo[4,5-b]pyridin-3-yl)benzyl)amino)methyl)phenyl)acetate AV1 (50.0 mg, 17.7% yield) as a yellow solid. ¹H NMR (400 MHz, DMSO-d₆) δ_{H}=3.61 (s, 3H) 3.68 (s, 2H) 3.72 (s, 2H) 3.78 (s, 2H) 6.41 (dd, J=7.6, 4.8 Hz, 1H), 7.01 (s, 2H), 7.16-7.12 (m, 1H), 7.21 (dd, J=7.6, 1.8 Hz, 1H), 7.31-7.26 (m, 3H), 7.57-7.37 (m, 8H), 8.06-7.98 (m, 4H), 8.27 (d, J=8.0 Hz, 1H).

Step 2. To a solution of methyl 2-(3-(((4-(2-(2-aminopyridin-3-yl)-5-phenyl-3H-imidazo[4,5-b]pyridin-3-yl)benzyl)amino)methyl)phenyl)acetate AV1 (50.0 mg, 0.0900 mmol) in THF (0.500 mL) and water (0.500 mL), LiOH·H₂O (11.0 mg, 0.450 mmol) was added and the reaction mixture was stirred at 20° C. for 2 hrs. The reaction mixture was concentrated and purified by prep-HPLC (column: Welch Xtimate C18 150*40 mm*10 μm; mobile phase: [water (NH₄HCO₃)-ACN]; B %: 4%-44%, 32 min) to afford 2-(3-(((4-(2-(2-aminopyridin-3-yl)-5-phenyl-3H-imidazo[4,5-b]pyridin-3- yl)benzyl)amino)methyl)phenyl)acetic acid 47 (27.0 mg, 54.9% yield) as a white solid. ¹H NMR (400 MHz, DMSO-d₆) δ_{H}=8.27 (d, J=8.4 Hz, 1H), 8.07-7.94 (m, 4H), 7.59-7.37 (m, 7H), 7.30-7.10 (m, 5H), 7.01 (s, 2H), 6.41 (dd, J=7.6, 4.8 Hz, 1H), 3.72 (s, 2H), 3.79 (s, 2H), 3.56 (s, 2H). HPLC Rₜ=2.046 min in 8 min chromatography, ACE Excel 3 C18 4.6*100 mm, purity 98.0%. LCMS Rₜ=1.143 min in 4 min chromatography, purity 97.7%, MS ESI calcd, for 540.23 [M+H]⁺ 541.23, found 541.2.

### Example 48. Synthesis of 2-(3-(((5-(2-(2-aminopyridin-3-yl)-5-phenyl-3H-imidazo[4,5-b]pyridin-3-yl)pyridin-2-yl)methyl)carbamoyl)phenyl)acetic acid (Compound 48)

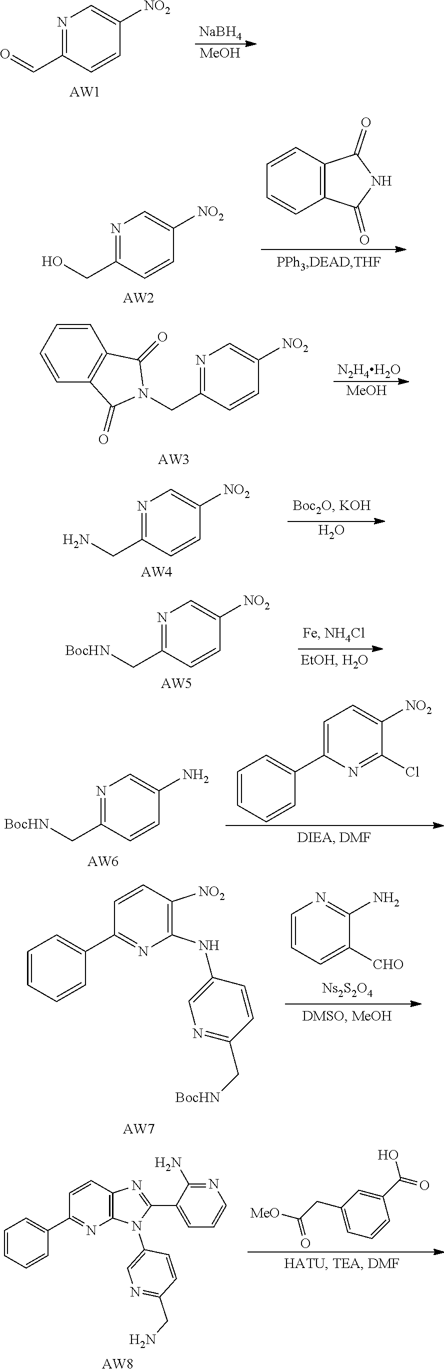

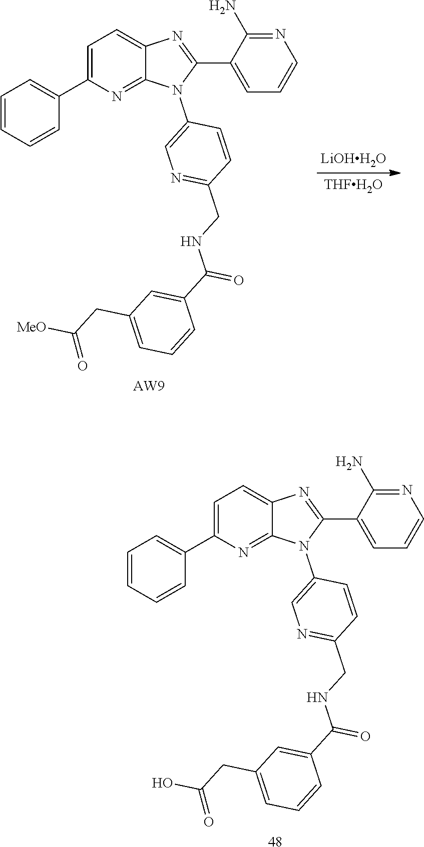

Step 1. In a three-neck flask (250 mL), to a solution of 5-nitropyridine-2-carbaldehyde AW1 (3.60 g, 23.7 mmol) in methanol (40.0 mL), NaBH₄ (985 mg, 26.0 mmol) was added at 0° C., and the mixture was stirred at 20° C. for 1 hr. The mixture was concentrated, and the residue was dissolved with EtOAc (50 mL), washed with brine (10 mL×3), dried over Na₂SO₄, filtered, and concentrated to afford (5-nitro-2-pyridyl) methanol AW2 (2.20 g, 14.3 mmol, 60.3% yield) as a yellow solid. ¹H NMR (DMSO-d₆, 400 MHz) δ_{H}=9.28 (d, J=2.4 Hz, 1H), 8.61 (dd, J=2.8, 8.8 Hz, 1H), 7.75 (d, J=8.8 Hz, 1H), 5.77 (t, J=5.6 Hz, 1H), 4.71-4.68 (m, 2H).

Step 2. To a solution of (5-nitro-2-pyridyl) methanol AW2 (2.25 g, 14.6 mmol), PPh₃ (4.59 g, 17.5 mmol) and isoindoline-1,3-dione (2.15 g. 14.6 mmol) in THF (50.0 mL), DEAD (3.05 g, 17.5 mmol) was added under N₂, and the mixture was stirred at 20° C. for 16 hrs. The mixture was concentrated to remove THE, dissolved with water, and extracted with EtOAc (50 mL). The organic phase was washed with brine (50 mL), dried with Na₂SO₄, filtered, and concentrated. The residue was purified by prep-HPLC (Welch Xtimate C18 150*30 mm*5 μm, water (FA)-ACN) to afford 2-[(5-nitro-2-pyridyl)methyl]isoindoline-1,3-dione AW3 (6.40 g, impure) as a yellow solid. LCMS Rₜ=0.457 min in 1 min chromatography, 0-95AB_220&254_Agilent.M purity 36.7%, MS ESI calcd, for 283.06 [M+H]⁺ 284.06, found 284.0.

Step 3. In a three-neck flask (250 mL), to a solution of 2-1 (5-nitro-2-pyridyl)methyl]isoindoline-1,3-dione AW3 (3.00 g, 10.6 mmol) in methanol (100 mL), N₂H₄·H₂O) (2.15 g, 42.4 mmol) was added, and the mixture was stirred at 20° C. for 16 hrs. The solution was concentrated under reduced pressure, the residue was adjusted to pH ~3 with 1 M HCl, and subsequently washed with EtOAc (20 mL×3). The water phase was adjusted to pH ~10 with KOH, washed with DCM (20 mL×3), and the water phase was used in the next step directly. LCMS Rₜ=0.14 min in 1 min chromatography, 5-95AB_220&254_Agilent.M, MS ESI calcd. for 153.05 [M+H]⁺ 154.05, found 154.1.

Step 4. To a solution of (5-nitro-2-pyridyl) methanamine AW4 (800 mg, 5.22 mmol) in water (100 mL), Boc₂O (11.4 g, 52.2 mmol) and KOH (586 mg, 10.4 mmol) were added, and the mixture was stirred at 20° C. for 16 hrs. The mixture was extracted with DCM (30 mL×3), dried with Na₂SO₄, and concentrated. The residue was purified by column (EtOAc in PE, 0-30%) to afford tert-butyl N—[(S-nitro-2-pyridyl)methyl]carbamate AW5 (370 mg, 1.46 mmol, 27.9% yield) as a yellow oil. ¹H NMR (CHLOROFORM-d, 400 MHz) δ_{H}=9.36 (d, J=2.4 Hz, 1H), 8.45 (dd, J=2.4, 8.8 Hz, 1H), 7.49 (d, J=8.8 Hz, 1H), 5.48 (br s, 1H), 4.56 (br d, J=5.6 Hz, 2H), 1.47 (s, 9H).

Step 5. To a solution of tert-butyl N-[(5-nitro-2-pyridyl)methyl]carbamate AW5 (370 mg, 1.46 mmol) in ethanol (5.00 mL) and water (1.00 mL), NH₄Cl (156 mg, 2.92 mmol) and Fe (327 mg, 5.84 mmol) were added, and the mixture was stirred at 90° C. for 2 hrs. The mixture was filtered, washed with DCM (5 mL) and EtOH (5 mL), and the organic phase was concentrated. The residue was diluted with water (5 mL), extracted with EtOAc (5 mL). The organic phase was concentrated to afford tert-butyl N-[(5-amino-2-pyridyl)methyl]carbamate AW6 (350 mg, crude) as a yellow solid. ¹H NMR (DMSO-d₆ 400 MHz) δ_{H}=7.83 (s, 1H), 7.17 (br t, J=5.2 Hz, 1H), 6.95-6.85 (m, 2H), 5.15 (s, 2H), 4.03 (br d, J=5.6 Hz, 2H), 1.39 (s, 9H).

Step 6. To a solution of tert-butyl N-[(5-amino-2-pyridyl)methyl]carbamate AW6 (350 mg, 1.57 mmol) and 2-chloro-3-nitro-6-phenyl-pyridine (368 mg, 1.57 mmol) in DMF (10.0 mL), DIEA (0.820 mL, 4.70 mmol) was added and the mixture was stirred at 110° C. for 16 hrs. Water (50 mL) was then added, and the mixture was extracted with EtOAc (50 mL×3). The organic phase was washed with brine (20 mL×3), dried with Na₂SO₄ and concentrated. The residue was purified by column (EtOAc in PE, 0-40%) to give tert-butyl N-[[5-[(3-nitro-6-phenyl-2-pyridyl)amino]-2-pyridyl]methyl]carbamate AW7 (360 mg, 0.854 mmol, 54.5% yield) as a brown solid. ¹H NMR (DMSO-d₆ 400 MHz) δ_{H}=10.11 (s, 1H), 8.83 (d, J=2.4 Hz, 1H), 8.62 (d, J=8.8 Hz, 1H), 8.17-7.97 (m, 3H), 7.62 (d, J=8.8 Hz, 1H), 7.56-7.48 (m, 3H), 7.48-7.41 (m, 1H), 7.33 (d, J=8.8 Hz, 1H), 4.25 (br d, J=6.4 Hz, 2H), 1.42 (s, 9H).

Step 7. To a stirred solution of 2-aminopyridine-3-carbaldehyde (130 mg, 1.07 mmol), tert-butyl N-[[5-[(3-nitro-6-phenyl-2-pyridyl)amino]-2-pyridyl]methyl]carbamate AW7 (300 mg, 0.712 mmol) in DMSO (5.00 mL) and methanol (0.500 mL), Na₂S₂O₄ (372 mg, 2.14 mmol) was added, and the mixture was stirred at 100° C. under N₂ for 12 hrs. The mixture was filtered, and the filtrate was purified by prep-HPLC (YMC Triart 30*150 mm*5 μm, water (FA)-ACN) to afford 3-[3-[6-(aminomethyl)-3-pyridyl]-5-phenyl-imidazo[4,5-b]pyridin-2-yl]pyridin-2-amine AW8 (50.0 mg, 0.127 mmol, 17.9% yield) as a yellow solid. ¹H NMR (DMSO-d₆ 400 MHz) δ_{H}=8.69 (s. 1H), 8.30 (d, J=8.8 Hz, 1H), 8.25 (s, 1H), 8.08-8.00 (m, 4H), 7.98 (dd, J=2.4, 8.4 Hz, 1H), 7.66 (d, J=8.4 Hz, 1H), 7.51-7.40 (m, 4H), 7.36 (dd, J=2.4, 7.6 Hz, 1H), 6.73 (s, 2H), 6.50 (dd, J=5.2, 7.6 Hz, 1H), 4.06 (s, 2H).

Step 8. To a solution of 3-(2-methoxy-2-oxo-ethyl)benzoic acid (27.0 mg, 0.140 mmol) in DMF (1.00 mL), TEA (0.035 mL, 0.254 mmol) and HATU (53.0 mg, 0.140 mmol) were added, and the mixture was stirred at 20° C. for 0.5 hrs. To the mixture, a solution of 3-[3-[6-(aminomethyl)-3-pyridyl]-5-phenyl-imidazo[4,5-b]pyridin-2-yl]pyridin-2-amine AW8 (50.0 mg, 0.127 mmol) and TEA (0.0350 mL, 0.254 mmol) in DMF (1.00 mL) were added and the mixture was stirred at 20° C. for 2 hrs. The mixture was quenched with water (5 mL), extracted with EtOAc (5.0 mL), and the organic phase was washed with brine (5.0 mL), dried with Na₂SO₄, and concentrated. The residue was purified by column (EtOAc in MeOH, 0-30%) to afford methyl 2-(3-(((5-(2-(2-aminopyridin-3-yl)-5-phenyl-3H-imidazo[4,5-b]pyridin-3-yl)pyridin-2-yl)methyl)carbamoyl) phenyl)acetate AW9 (40.0 mg, 0.0702 mmol, 55.3% yield) as a yellow solid. LCMS RF 0.457 min in 1 min chromatography, 5-95AB_220&254_Agilent.M, purity 92.3%, MS ESI calcd, for 569.22 [M+H]⁺ 570.22, found 570.1.

Step 9. To a solution of methyl 2-(3-(((5-(2-(2-aminopyridin-3-yl)-5-phenyl-3H-imidazo[4,5-b]pyridin-3-yl)pyridin-2-yl)methyl)carbamoyl)phenyl)acetate AW9 (40.0 mg, 0.0702 mmol) in THF (1.00 mL) and water (1.00 mL), LiOH·H₂O (8.80 mg, 0.211 mmol) was added and the mixture stirred at 25° C. for 16 hrs. The mixture was concentrated and the resulting residue was adjusted to pH ~6 and purified by prep-HPLC (Welch Xtimate C18 150*30 mm*5 μm, water (FA)-ACN) to afford 2-(3-(((5-(2-(2-aminopyridin-3-yl)-5-phenyl-3H-imidazo[4,5-b]pyridin-3-yl)pyridin-2-yl)methyl)carbamoyl)phenyl)acetic acid 48 (19.4 mg, 0.0347 mmol, 49.5% yield, 99.5% purity) as a white solid. ¹H NMR (DMSO-d₆ 400 MHz) δ_{H}=12.71-12.10 (m, 1H), 9.20 (t. J=5.6 Hz, 1H), 8.67 (d, J=2.4 Hz, 1H), 8.29 (d, J=8.8 Hz, 1H), 8.08-7.99 (m, 4H), 7.95 (dd, J=2.4, 8.4 Hz, 1H), 7.86-7.81 (m, 2H), 7.54-7.31 (m, 7H), 6.74 (s, 2H), 6.50 (dd, J=4.8, 7.6 Hz, 1H), 4.68 (d, J=6.4 Hz, 2H), 3.65 (s, 2H). HPLC Rₜ=8.795 min in 15 min chromatography. 0-60AB, purity 99.5%. LCMS Rₜ=2.449 min in 4 min chromatography. Xtimate C18 2.1*30 mm, 3 μm, purity 100%, MS ESI calcd, for 555.20 [M+H]⁺ 556.20, found 556.1.

### Example 49. Synthesis of 2-(3-(((6-(2-(2-aminopyridin-3-yl)-5-phenyl-3H-imidazo[4,5-b]pyridin-3-yl)pyridin-3-yl)methyl)carbamoyl)phenyl)acetic acid (Compound 49)

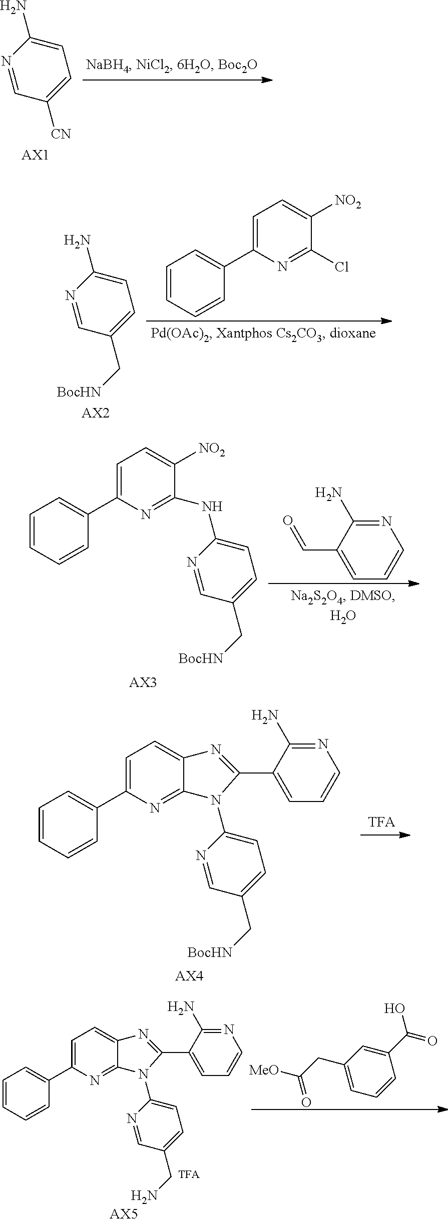

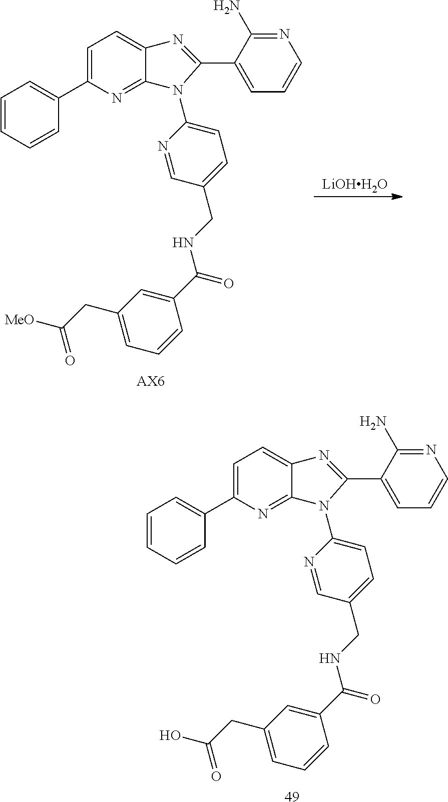

Step 1. To a solution of 6-aminopyridine-3-carbonitrile AX1 (5.00 g, 42.0 mmol) in methanol (250 mL), NiCl₂·6H₂O (998 mg, 4.20 mmol). Boc₂O (18.3 g, 83.9 mmol), and then NaBH₄ (9.53 g, 252 mmol) were added to the reaction mixture slowly at 0° C., and the reaction mixture stirred at 0° C. to 25° C. for 16 hrs. The reaction mixture was poured slowly into ice water (200 mL) and concentrated to remove MeOH. The resulting mixture was extracted with EtOAc (150 mL×3), and the combined organic phase washed with brine (50 mL), water (50 mL), dried over anhydrous Na₂SO₄, filtered and concentrated. The residue was purified by flash silica gel chromatography (eluent: 0-4% of MeOH in DCM) to give tert-butyl N-[(6-amino-3-pyridyl)methyl]carbamate AX2 (1.80 g, 19.2% yield) as a yellow oil. LCMS Rₜ=0.345 min in 1.0 min chromatography, purity 67.3%, MS ESI calcd, for 223.14 [M+H]⁺ 224.14, found 224.1.

Step 2. To a solution of 2-chloro-3-nitro-6-phenyl-pyridine (1.80 g, 7.67 mmol) and tert-butyl N-[(6-amino-3-pyridyl)methyl]carbamate AX2 (1.80 g, 8.06 mmol) in 1,4-dioxane (30.0 mL), Xantphos (444 mg, 0.767 mmol), Cs₂CO₃ (7.50 g, 23.0 mmol) and Pd(OAc)₂ (172 mg, 0.767 mmol) were added at 25° C. and the mixture stirred at 90° C. for 16 hrs. The reaction mixture was concentrated, water (100 mL) was added to the residue and the resulting mixture extracted with EtOAc (50 mL×3). The combined organic phase was washed with brine (20 mL), water (20 mL), dried over anhydrous Na₂SO₄, filtered and concentrated. The residue was purified by flash silica gel chromatography (eluent: 0-37% of EtOAc in PE) to give tert-butyl N-[6-[(3-nitro-6-phenyl-2-pyridyl)amino]-3-pyridyl]methyl]carbamate AX3 (2.30 g, 71.1% yield) as a yellow solid. LCMS Rₜ=0.639 min in 1.0 min chromatography, purity 97.2%, MS ESI calcd, for 421.18 [M+H]⁺ 422.18, found 422.1.

Step 3. To a solution of tert-butyl N-[[6-[(3-nitro-6-phenyl-2-pyridyl)amino]-3-pyridyl]methyl]carbamate AX3 (1.00 g. 2.37 mmol) in DMSO (23.0 mL) and methanol (23.0 mL), 2-aminopyridine-3-carbaldehyde (319 mg, 2.61 mmol) and Na₂S₂O₄ (1.03 g, 5.93 mmol) were added at 25° C., and the mixture stirred at 100° C. for 16 hrs. The reaction mixture was concentrated to remove MeOH, water (50 mL) was added to the residue and the resulting mixture extracted with EtOAc (100 mL×3). The combined organic phase was washed with brine (20 mL), water (20 mL), dried over anhydrous Na₂SO₄, filtered and concentrated. The residue was purified by flash silica gel chromatography (eluent: 0-49% of EtOAc in PE) to give tert-butyl ((6-(2-(2-aminopyridin-3-yl)-5-phenyl-3H-imidazo[4,5-b]pyridin-3-yl)pyridin-3-yl)methyl)carbamate AX4 (420 mg, 45.0% yield) as a yellow solid. ¹H NMR (400 MHz, DMSO-d₆) δ=8.55 (d, J=8.4 Hz, 2H), 8.43 (d, J=4.0 Hz, 1H), 8.23 (s, 1H), 8.17 (d, J=7.6 Hz, 2H), 7.94 (d, J=8.4 Hz, 1H), 7.67 (d, J=8.8 Hz, 1H), 7.53 (t, J=7.2 Hz, 2H), 7.48-7.38 (m, 2H), 7.12 (dd, J=4.8, 7.6 Hz, 1H), 4.11 (d, J=6.0 Hz, 2H), 1.40 (s, 9H).

Step 4. To a solution of tert-butyl ((6-(2-(2-aminopyridin-3-yl)-5-phenyl-3H-imidazo[4,5-b]pyridin-3-yl)pyridin-3-yl)methyl)carbamate AX4 (100 mg, 0.203 mmol) in DCM (4.00 mL), TFA (2.00 mL) was added at 25° C., and the mixture was stirred for 2 hrs. The reaction mixture was concentrated to give 3-[3-[5-(aminomethyl)-2-pyridyl]-5-phenyl-imidazo[4,5-b]pyridin-2-yl]pyridin-2-amine trifluoroacetate AX5 (79.0 mg, 99.1% yield) as a brown oil, which was used in the next step directly. LCMS Rₜ=0.480 min in 1.0 min chromatography, purity 69.4%, MS ESI calcd, for 393.17 [M+H]⁺ 394.17, found 394.1.

Step 5. To a solution of 3-(2-methoxy-2-oxoethyl)benzoic acid (148 mg, 0.762 mmol) in DMF (4.00 mL), DIEA (494 mg, 3.82 mmol), HATU (727 mg, 1.91 mmol) and 3-[3-[5-(aminomethyl)-2-pyridyl]-5-phenyl-imidazo[4,5-b]pyridin-2-yl]pyridin-2-amine trifluoroacetate AX5 (200 mg, 0.510 mmol) were added at 25° C., and the mixture was stirred at 25° C. for 2 hrs. Aq. LiCl (20 mL, 3%) was added to the residue, and the resulting mixture was extracted with EtOAc (10 mL×3). The combined organic phase was washed with brine (5 mL), water (5 mL), dried over anhydrous Na₂SO₄, filtered and concentrated. The residue was purified by flash silica gel chromatography (eluent with 0-11% of MeOH in DCM) to give methyl 2-(3-(((6-(2-(2-aminopyridin-3-yl)-5-phenyl-3H-imidazo[4,5-b]pyridin-3-yl)pyridin-3-yl)methyl)carbamoyl)phenyl)acetate AX6 (100 mg, 34.5% yield) as a yellow solid. ¹H NMR (400 MHz, DMSO-d₆) δ=9.08 (t . . . )=6.0 Hz, 1H), 8.26 (d, J=8.4 Hz, 1H), 8.05-7.96 (m, 5H), 7.85 (d, J=8.4 Hz, 2H), 7.41-7.26 (m, 7H), 7.21 (dd, J=7.6, 2.0 Hz, 1H), 6.95 (s, 2H), 6.41 (dd, J=7.6, 4.8 Hz, 1H), 4.60 (d, J=6.0 Hz, 2H), 4.05-4.02 (m, 2H), 2.69 (s, 3H).

Step 6. To a solution of methyl 2-(3-(((6-(2-(2-aminopyridin-3-yl)-5-phenyl-3H-imidazo[4,5-b]pyridin-3-yl)pyridin-3-yl)methyl)carbamoyl)phenyl)acetate AX6 (100 mg, 0.176 mmol) in THF (2.50 mL) and water (2.50 mL), LiOH·H₂O (14.0 mg, 0.351 mmol) was added at 25° C., and the mixture was stirred for 2 hrs. The reaction mixture was concentrated to remove THE. The residue was purified by prep-HPLC (Column: Welch Xtimate C18 150*30 mm*5 μm; Condition: water (NH₄HCO₃)-ACN; begin b: 6-46%) to give 2-(3-(((6-(2-(2-aminopyridin-3-yl)-5-phenyl-3H-imidazo[4,5-b]pyridin-3-yl)pyridin-3-yl)methyl)carbamoyl)phenyl)acetic acid 49 (10.0 mg, 10.5% yield) as a yellow solid. ¹H NMR (400 MHz, DMSO-d₆) δ=12.48 (s, 1H), 9.04 (t, J=6.0 Hz, 1H), 8.57 (d, J=8.4 Hz, 2H), 8.41 (dd, J=4.8, 1.6 Hz, 1H), 8.33 (d, J=2.0 Hz, 1H), 8.22-8.14 (m, 3H), 7.91 (d, J=8.4 Hz, 1H), 7.80-7.74 (m, 3H), 7.56-7.49 (m, 2H), 7.47-7.38 (m, 3H), 7.11 (dd, J=7.6, 4.8 Hz, 1H), 4.46 (d, J=6.0 Hz, 2H), 3.62 (s, 2H). HPLC Rₜ=4.172 min in 8 min chromatography, purity 99.1%. LCMS Rₜ=1.890 min in 4 min chromatography, purity 99.7%, MS ESI calcd. for 555.21 [M+H]⁺ 556.21, found 556.2.

### Example 50. Synthesis of 2-(3-((4-(2-(2-aminopyridin-3-yl)-5-phenyl-3H-imidazo[4,5-b]pyridin-3-yl)-2-fluorobenzyl)carbamoyl)phenyl)acetic acid (Compound 50)

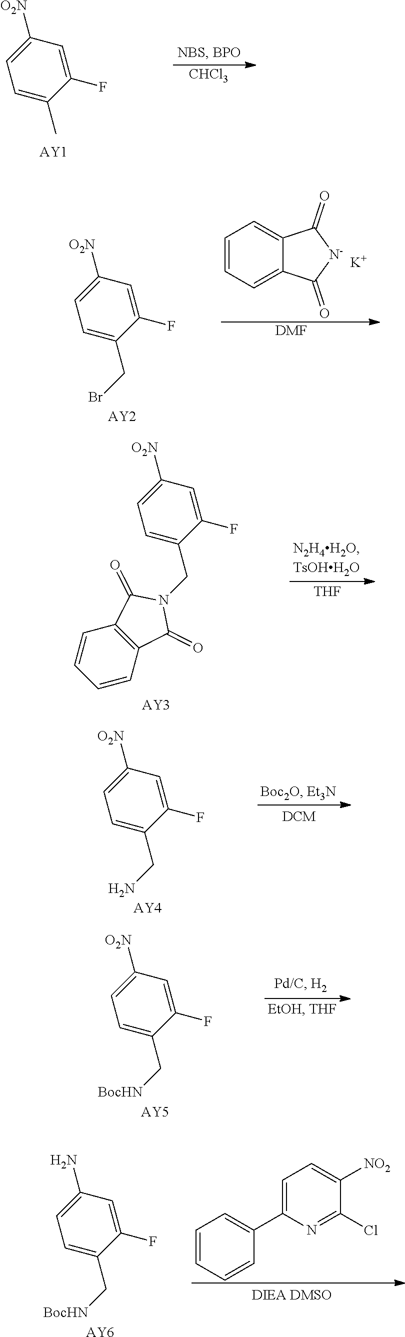

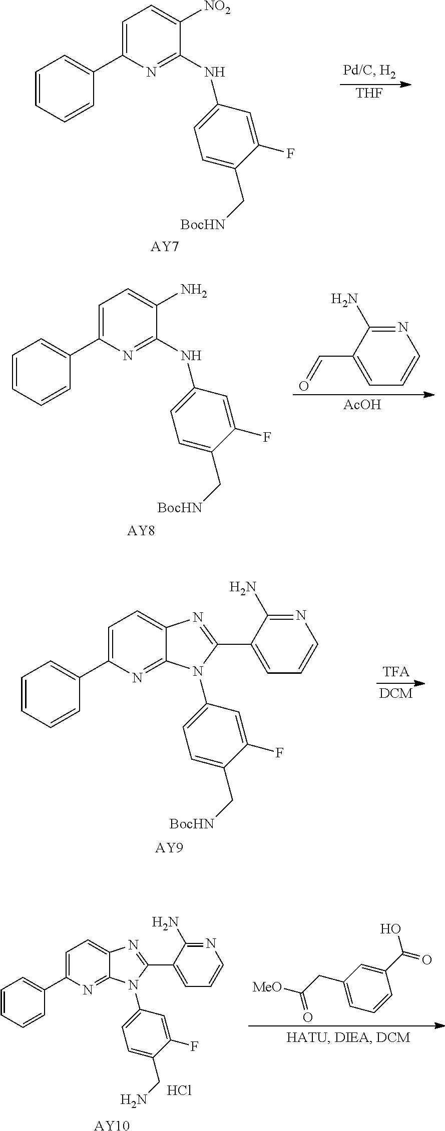

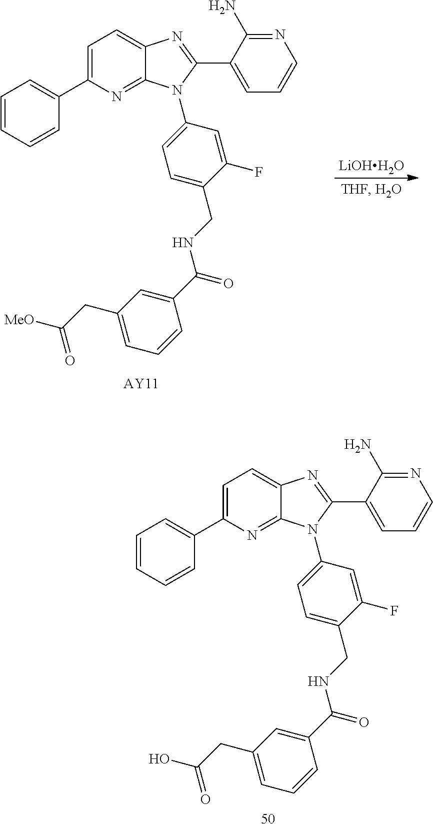

Step 1. To a solution of 2-fluoro-1-methyl-4-nitrobenzene AY1 (4.00 g, 25.8 mmol) in chloroform (80.0 mL), BPO (0.62 g, 2.58 mmol) and NBS (4.59 g, 25.8 mmol) were added, and the mixture was stirred at 80° C. for 12 hrs. Water (20 mL) was added and the resulting mixture was extracted with EtOAc (100 mL×3). The combined organic phase was washed with brine (20 mL), dried over anhydrous Na₂SO₄, filtered and concentrated. The residue was purified by flash silica gel chromatography (eluent with 0-5% of EtOAc in PE) to afford 1-(bromomethyl)-2-fluoro-4-nitrobenzene AY2 (4.75 g. 20.3 mmol) as a yellow oil. ¹H NMR (400 MHz, DMSO-d₆) δ=8.19-8.08 (m, 2H), 7.86 (t, J=8.0 Hz, 1H), 4.79 (s, 2H).

Step 2. To a solution of 1-(bromomethyl)-2-fluoro-4-nitrobenzene AY2 (4.75 g, 20.3 mmol) in DMF (50.0 mL), phthalimide potassium salt (7.52 g, 40.6 mmol) was added, and the mixture was stirred at 25° C. for 12 hrs. The reaction mixture was diluted with EtOAc (80 mL) and washed with brine (20 mL×2). The combined organic phase was dried over anhydrous Na₂SO₄, filtered and concentrated. The residue was purified by flash silica gel chromatography (eluent of 0-20% EtOAc in PE) to afford 2-(2-fluoro-4-nitrobenzyl) isoindoline-1,3-dione AY3 (4.00 g, 13.3 mmol) as a yellow solid. ¹H NMR (400 MHz, DMSO-de) 8=8.13 (dd, J=9.6, 2.0 Hz, 1H), 8.03 (dd, J=8.4, 2.0 Hz, 1H), 7.83 (s, 4H), 7.67 (t, J=8.0 Hz, 1H), 4.92 (s, 2H).

Step 3. To a solution of 2-(2-fluoro-4-nitrobenzyl) isoindoline-1,3-dione AY3 (4.00 g, 13.3 mmol) in THF (70.0 mL), N₂H₄·H₂O (2.70 g, 53.3 mmol) and TsOH·H₂O (0.25 g, 1.33 mmol) were added and the mixture was stirred at 66° C. for 6 hrs. The reaction mixture was diluted with EtOAc (80 mL) and washed with water (20 mL×2). The combined organic phase was washed with brine (20 mL), dried over anhydrous Na₂SO₄, filtered and concentrated to afford (2-fluoro-4-nitrophenyl) methanamine AY4 (1.20 g, 7.05 mmol) as a brown oil. ¹H NMR (400 MHz, DMSO-d₆) δ=8.11-8.05 (m, 1H), 8.04-7.97 (m, 1H), 7.81 (t, J=8.4 Hz, 1H), 3.85 (s, 2H).

Step 4. To a solution of (2-fluoro-4-nitrophenyl) methanamine AY4 (1.20 g, 7.05 mmol) in DCM (20.0 mL), TEA (2.14 g, 21.2 mmol), and then Boc₂O (3.08 g, 14.1 mmol) were added, and the mixture stirred at 25° C. for 12 hrs. The reaction mixture was diluted with EtOAc (50 mL) and washed with sat aq. NH₄Cl (10 mL×2). The combined organic phase was washed with brine (10 mL), dried over anhydrous Na₂SO₄, filtered and concentrated. The residue was purified by flash silica gel chromatography (eluent with 0-5% of EtOAc in PE) to afford tert-butyl(2-fluoro-4-nitrobenzyl)carbamate AY5 (1.70 g, 6.29 mmol) as a yellow oil. ¹H NMR (400 MHz, DMSO-d₆) δ=8.13-8.05 (m, 2H), 7.60-7.53 (m, 2H), 4.26 (d, J=6.0 Hz, 2H), 1.39 (s, 9H).

Step 5. To a solution of tert-butyl(2-fluoro-4-nitrobenzyl)carbamate AY5 (1.70 g, 6.29 mmol) in THF (20.0 mL) and ethanol (20.0 mL), Pd/C (wet, 10%, 1.20 g) was added under N₂ atmosphere, the suspension was degassed and purged with H₂ (×3), and the reaction mixture was stirred under H₂ (15 Psi) at 25° C. for 12 hrs. The reaction mixture was filtered, and the filtrate was concentrated to dryness to afford ter-butyl (4-amino-2-fluorobenzyl)carbamate AY6 (1.30 g, 5.41 mmol) as a yellow oil. ¹H NMR (400 MHz, DMSO-de) 8=7.16-7.08 (m, 1H), 6.92 (t, J=8.4 Hz, 1H), 6.38-6.20 (m, 2H), 5.26 (s, 2H), 3.97 (br d, J=5.6 Hz, 2H), 1.37 (s, 9H).

Step 6. To a solution of 2-chloro-3-nitro-6-phenylpyridine (1.00 g, 4.28 mmol) in DMSO (20.0 mL), DIEA (1.66 g, 12.8 mmol) and tert-butyl (4-amino-2-fluorobenzyl)carbamate AY6 (1.23 g, 5.14 mmol) were added, and the mixture was stirred at 100° C. for 12 hrs. The reaction mixture was diluted with EtOAc (60 mL) and washed with brine (10 mL×3). The combined organic phase was dried over anhydrous Na₂SO₄, filtered and concentrated. The residue was purified by flash silica gel chromatography (eluent of 0-20% EtOAc in PE) to afford tert-butyl(2-fluoro-4-((3-nitro-6-phenylpyridin-2-yl)amino)benzyl)carbamate A Y⁷ (1.10 g, 2.51 mmol) as a yellow oil. ¹H NMR (400 MHz, DMSO-d₆) δ=10.13 (s, 1H), 8.63 (d, J=8.4 Hz, 1H), 8.14-8.08 (m, 2H), 7.76 (br d, J=12.4 Hz, 1H), 7.64 (d, J=8.8 Hz, 1H), 7.58-7.49 (m, 5H), 7.36-7.30 (m, 1H), 4.19 (br d, J=5.6 Hz, 2H), 1.41 (s, 9H).

Step 7. To a solution of tert-butyl(2-fluoro-4-((3-nitro-6-phenylpyridin-2-yl)amino)benzyl)carbamate AY7 (1.10 g, 2.51 mmol) in THF (20.0 mL), Pd/C (wet, 10%, 0.500 g) under N₂ atmosphere was added, the suspension was degassed and purged with H₂ (×3), and the reaction mixture was stirred under H₂ (15 Psi) at 25° C. for 12 hrs. The reaction mixture was filtered, and the filtrate was concentrated to dryness to afford tert-butyl (4-((3-amino-6-phenylpyridin-2-yl)amino)-2-fluorobenzyl)carbamate AY8 (950 mg, 2.33 mmol) as a yellow solid. ¹H NMR (400 MHz, DMSO-d₆) δ=8.08 (s, 1H), 7.94-7.91 (m, 2H), 7.85 (br d, J=13.6 Hz, 1H), 7.43-7.36 (m, 3H), 7.31-7.16 (m, 5H), 7.01 (d, J=8.0 Hz, 1H), 4.13 (br d, J=5.6 Hz, 2H), 1.40 (s, 9H).

Step 8. To a solution of tert-butyl (4-((3-amino-6-phenylpyridin-2-yl)amino)-2-fluorobenzyl)carbamate AY8 (900 mg, 2.20 mmol) in AcOH (15.0 mL), 2-aminonicotinaldehyde (323 mg, 2.64 mmol) was added, and the mixture stirred at 110° C. for 1 hr. The reaction mixture was concentrated directly, and the residue was purified by flash silica gel chromatography (eluent with 0-3% of MeOH in DCM) to afford tert-butyl (4-(2-(2-aminopyridin-3-yl)-5-phenyl-3H-imidazo[4,5-b]pyridin-3-yl)-2-fluorobenzyl)carbamate AY9 (750 mg, 1.47 mmol) as a yellow solid. ¹H NMR (400 MHz, DMSO-d₆) δ=8.27 (d, J=8.4 Hz, 1H), 8.06-8.00 (m, 4H), 7.50-7.40 (m, 6H), 7.31-7.26 (m, 2H), 6.89 (s, 2H), 6.50-6.43 (m, 1H), 4.27 (d, J=6.0 Hz, 2H), 1.41 (s, 9H).

Step 9. To a solution of tert-butyl (4-(2-(2-aminopyridin-3-yl)-5-phenyl-3H-imidazo[4,5-b]pyridin-3-yl)-2-fluorobenzyl)carbamate AY9 (750 mg, 1.47 mmol) in DCM (9.0 mL), TFA (1.0 mL, 1.47 mmol) was added and the mixture stirred at 25° C. for 1 hr. The reaction mixture was concentrated directly to afford 3-(3-(4-(aminomethyl)-3-fluorophenyl)-5-phenyl-3H-imidazo[4,5-b]pyridin-2-yl)pyridin-2-amine hydrochloride AY10 (600 mg, 1.46 mmol) as a brown oil.

Step 10. To a solution of 3-(3-(4-(aminomethyl)-3-fluorophenyl)-5-phenyl-3H-imidazo[4,5-b]pyridin-2-yl)pyridin-2-amine hydrochloride AY10 (200 mg, 0.487 mmol) in DCM (10.0 mL), DIEA (189 mg, 1.46 mmol) was added. 3-(2-Methoxy-2-oxoethyl)benzoic acid (95.0 mg, 0.487 mmol) and HATU (220 mg, 0.585 mmol) were then added to the mixture, and the mixture stirred at 25° C. for 12 hrs. The reaction mixture was concentrated directly, and the resulting residue was purified by flash silica gel chromatography (eluent with 0-2% of MeOH in DCM) to afford methyl 2-(3-((4-(2-(2-aminopyridin-3-yl)-5-phenyl-3H-imidazo [4,5-b]pyridin-3-yl)-2-fluorobenzyl)carbamoyl)phenyl)acetate AY11 (120 mg, 0.205 mmol) as a yellow oil. ¹H NMR (400 MHz, DMSO-d₆) δ=9.11 (t, J=5.6 Hz, 1H), 8.38-8.25 (m, 2H), 8.06-7.99 (m, 4H), 7.85-7.80 (m, 2H), 7.52-7.44 (m, 6H), 7.32-7.28 (m, 2H), 6.86 (s, 2H), 6.51-6.44 (m, 1H), 4.61 (d, J=5.6 Hz, 2H), 3.76 (s, 2H), 3.62 (s, 3H).

Step 11. To a solution of methyl 2-(3-((4-(2-(2-aminopyridin~3-yl)-5-phenyl-3H-imidazo[4,5-b]pyridin-3-yl)-2-fluorobenzyl)carbamoyl)phenyl)acetate AY11 (120 mg, 0.205 mmol) in THF (4.00 mL), water (1.00 mL) and LiOH·H₂O (9.00 mg, 0.205 mmol) were added, and the mixture stirred at 25° C. for 12 hrs. The reaction mixture was concentrated directly, and the resulting residue was purified by prep-HPLC (Column: Xtimate C18 150*40 mm*10 μm; mobile phase: [water (NH₄HCO₃)-ACN]; B %: 4%-44%, 32 min) to afford 2-(3-((4-(2-(2-aminopyridin-3-yl)-5-phenyl-3H-imidazo[4,5-b]pyridin-3-yl)-2-fluorobenzyl)carbamoyl)phenyl)acetic acid 50 (29.0 mg, 0.0498 mmol) as a white solid. ¹H NMR (400 MHz, DMSO-d₆) δ=9.11 (t, J=5.6 Hz, 1H), 8.27 (d, J=8.4 Hz, 1H), 8.06-7.98 (m, 4H), 7.97-7.90 (m, 1H), 7.84-7.78 (m, 2H), 7.53-7.37 (m, 7H), 7.32-7.27 (m, 2H), 6.85 (s, 2H), 6.47 (dd, J=4.8, 7.6 Hz, 1H), 4.61 (br d, J=5.6 Hz, 2H), 3.63 (s, 2H). HPLC Rₜ=2.167 min in 8 min chromatography, purity 100%. LCMS Rₜ=0.551 min in 2.5 min chromatography, purity 99.5%, MS ESI calcd, for 572.20 [M+H]⁺ 573.20, found 573.2.

### Example 51. Synthesis of 2-(3-((4-(2-(2-aminopyridin-3-yl)-6-phenyl-1H-benzo[d]imidazol-1-yl)benzyl)carbamoyl)phenyl)acetic acid (Compound 51)

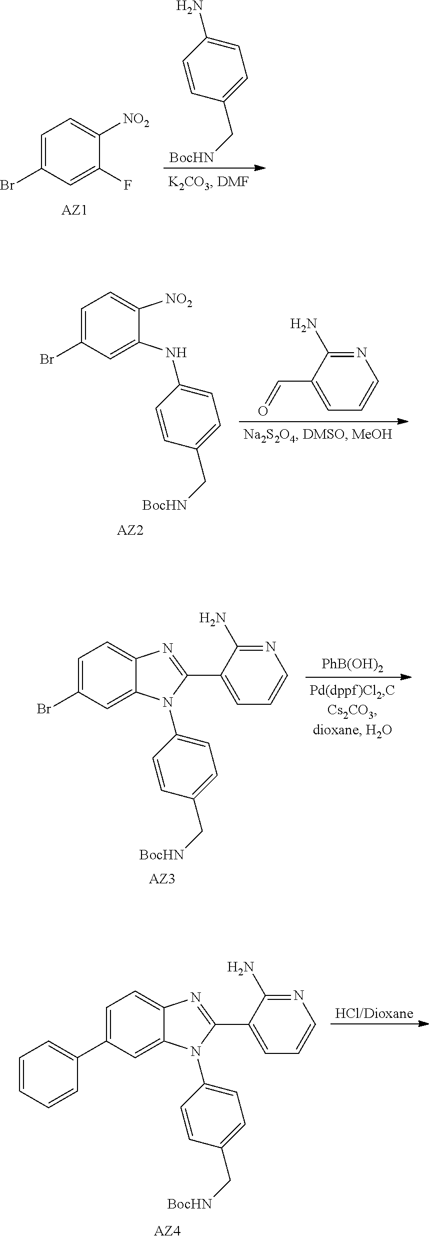

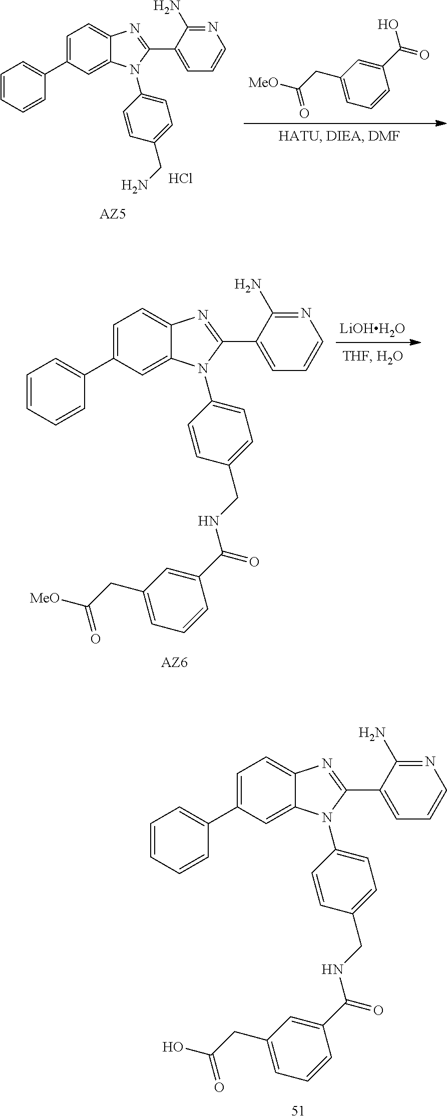

Step 1. To a solution of tert-butyl N-[(4-aminophenyl)methyl]carbamate (4.70 g, 21.5 mmol) and 4-bromo-2-fluoro-1-nitro-benzene AZ1 (4.50 g. 20.5 mmol) in DMF (20.0 mL), K₂CO₃ (5.65 mg, 40.9 mmol) was added and the mixture stirred at 90° C. for 5 hrs. The resulting mixture was allowed to cool to room temperature, diluted with EtOAc (100 mL) and washed with a 5% aq. LiCl solution (200× 3 mL). The organic layer was dried with Na₂SO₄, concentrated and the resulting residue was purified by column (15% of EtOAc in PE) to give tert-butyl N-[[4-(5-bromo-2-nitro-anilino)phenyl]methyl]carbamate AZ2 (2.58 g, 6.11 mmol, 29.9% yield) as a yellow solid. ¹H NMR (400 MHz, DMSO-d₆) δ=9.48 (s, 1H), 8.05 (d, J=9.2 Hz. 1H), 7.42 (s, 1H), 7.35-7.25 (m, 4H), 7.11 (d, J=1.6 Hz, 1H), 7.00 (dd, J=2.0, 8.8 Hz. 1H), 4.15 (d, J=6.0 Hz, 2H), 1.40 (s, 9H).

Step 2. To a stirred solution of 2-aminopyridine-3-carbaldehyde (195 mg, 1.60 mmol), tert-butyl N-[[4-(5-bromo-2-nitro-anilino)phenyl]methyl]carbamate AZ2 (500 mg, 1.18 mmol) in DMSO (10.0 mL) and methanol (2.00 mL), Na₂S₂O₄ (618 mg, 3.60 mmol) was added and the resulting mixture was stirred at 100° C. under N₂ for 12 hrs. The reaction mixture was diluted with EtOAc (50 mL) and washed with brine (50 mL×2), dried over anhydrous sodium sulfate, filtered and the filtrate was concentrated to afford a residue. The crude product was purified by flash chromatography (60% EtOAc in PE) to give tert-butyl N-[[4-[2-(2-amino-3-pyridyl)-6-bromo-benzimidazol-1-yl]phenyl methyl]carbamate AZ3 (400 mg, 0.810 mmol, 68.3% yield) as a yellow solid. LCMS Rₜ=0.5 min in 1.0 min chromatography, purity 98.1%, MS ESI calcd, for 493.11 [M+H]⁺ 494.11, found 494.1.

Step 3. To a stirred solution of phenylboronic acid (296 mg, 2.43 mmol), tert-butyl N-[[4-[2-(2-amino-3-pyridyl)-6-bromo-benzimidazol-1-yl]phenyl]methyl]carbamate AZ3 (400 mg, 0.810 mmol) and Cs₂CO₃ (791 mg, 2.43 mmol) in 1,4-dioxane (5.00 mL) and water (1.00 mL), Pd(dppf)Cl₂ (59.0 mg, 0.0810 mmol) was added under N₂ and the reaction stirred at 90° C. under N₂ for 12 hrs. The reaction mixture was concentrated to remove dioxane and diluted with EtOAc (10 mL), washed with brine (20 mL), dried over anhydrous sodium sulfate, filtered and the filtrate was concentrated and purified by flash chromatography on silica gel (70% of EtOAc in PE) to give tert-butyl N-[[4-[2-(2-amino-3-pyridyl)-6-phenyl-benzimidazol-1-yl]phenyl]methyl]carbamate AZ4 (150 mg, 0.310 mmol, 37.7% yield) as a yellow solid. LCMS Rₜ=0.550 min in 1.0 min chromatography, purity 97.1%, MS ESI calcd, for 491.23 [M+H]⁺ 492.23, found 492.2.

Step 4. To a stirred solution of tert-butyl N-[[4-[2-(2-amino-3-pyridyl)-6-phenyl-benzimidazol-1-yl]phenyl]methyl]carbamate AZ4 (150 mg, 0.310 mmol) in 1,4-dioxane (2.00 mL), HCl/dioxane (2.00 mL, 4 M) was added and the reaction mixture stirred at 25° C. under N₂ for 2 hrs. The reaction mixture was concentrated to afford 3-[1-[4-(aminomethyl)phenyl]-6-phenyl-benzimidazol-2-yl]pyridin-2-amine hydrochloride AZ5 (160 mg, crude) as a yellow solid. ¹H NMR (400 MHz, DMSO-d₆) δ=8.42 (d, J=2.4 Hz, 4H), 8.11 (dd, J=1.6, 6.0 Hz, 1H), 7.96 (d, J=8.4 Hz, 1H), 7.77-7.68 (m, 4H), 7.67-7.60 (m, 4H), 7.50-7.45 (m, 2H), 7.41-7.34 (m, 2H), 6.90-6.75 (m, 1H), 4.25-4.10 (m, 2H).

Step 5. To a stirred solution of 3-(2-methoxy-2-oxo-ethyl)benzoic acid (100 mg, 0.510 mmol) in DMF (3.00 mL), DIEA (0.230 mL, 1.23 mmol) and HATU (186 mg, 0.490 mmol) were added and the mixture stirred at 25° C. for 10 mins. 3-[1-[4-(Aminomethyl)phenyl]-6-phenyl-benzimidazol-2-yl]pyridin-2-amine hydrochloride AZ5 (160 mg, 0.409 mmol) was then added to the reaction mixture, and the resulting mixture was stirred at 25° C. under N₂ for 2 hrs. The reaction mixture was diluted with EtOAc (10 mL), washed with water (10 mL) and brine (20 mL), dried over anhydrous sodium sulfate, filtered and the filtrate was concentrated and purified by flash chromatography on silica gel (90% of EtOAc in PE) to give methyl 2-(3-((4-(2-(2-aminopyridin-3-yl)-6-phenyl-1H-benzo[d]imidazol-1-yl)benzyl)carbamoyl)phenyl)acetate AZ6 (120 mg, 0.210 mmol, 51.7% yield) as a yellow solid. LCMS Rₜ=0.505 min in 1.0 min chromatography, purity 95.4%, MS ESI calcd. for 567.23 [M+H]⁺ 568.23, found 568.2.

Step 6. To a stirred solution of methyl 2-(3-((4-(2-(2-aminopyridin-3-yl)-6-phenyl-1H-benzo[d]imidazol-1-yl)benzyl)carbamoyl)phenyl)acetate AZ6 (120 mg, 0.210 mmol) in THF (2.00 mL) and water (2.00 mL), LiOH·H₂O (17.0 mg, 0.420 mmol) was added and the reaction mixture was stirred at 25° C. for 2 hrs. The reaction was concentrated and diluted with MeOH (1 mL). The mixture was adjust to pH ~7 with 1N HCl aqueous solution and purified by prep-HPLC (column: Welch Xtimate C18 150*30 mm*5 μm, method: water (TFA)-ACN, begin B: 26, end B: 56) to afford 2-(3-((4-(2-(2-aminopyridin-3-yl)-6-phenyl-1H-benzo[d]imidazol-1-yl)benzyl)carbamoyl)phenyl)acetic acid 51 (35.0 mg, 0.0630 mmol, 29.9% yield) as an off-white solid. ¹H NMR (400 MHz, DMSO-d₆) δ=12.43 (s, 1H), 9.15 (t, J=5.6 Hz, 1H), 7.97 (dd, J=1.6, 4.8 Hz, 1H), 7.91-7.76 (m, 3H), 7.67-7.57 (m, 3H), 7.55-7.39 (m, 8H), 7.37-7.28 (m, 2H), 7.15 (dd, J=1.6, 7.6 Hz, 1H), 7.05 (s, 2H), 6.39 (dd, J=4.8, 7.6 Hz, 1H), 4.61 (d, J=5.6 Hz, 2H), 3.65 (s, 2H). HPLC Rₜ=3.819 min in 8 min chromatography, purity 98.7%. LCMS Rₜ=2.314 min in 4 min chromatography, purity 99.9%, MS ESI calcd, for 553.21 [M+H]⁺ 554.21, found 554.2.

### Example 52. Synthesis of 2-(3-((4-(2-(2-aminopyridin-3-yl)-5-phenyl-3H-imidazo[4,5-b]pyridin-3-yl)benzamido)methyl)phenyl)acetic acid (Compound 52) Synthesis of methyl 2-(3-(aminomethyl)phenyl)acetate hydrochloride

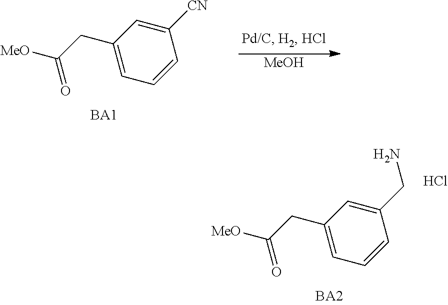 [SMILES: "MeOH" ; COC(=O)Cc1cccc(C#N)c1 ; COC(=O)Cc1cccc(CN)c1 ; Cl]

Step 1. To a solution of methyl 2-(3-cyanophenyl)acetate BA1 (500 mg, 2.85 mmol) in methanol (20.0 mL), HCl/dioxane (1.00 mL, 4 M) and Pd/C (150 mg, wet. 10.0%) were added. The reaction mixture was degassed, purged with H₂ (>3), and the mixture was stirred at 25° C. for 12 hrs under H₂ (1.00 MPa). The mixture was filtered through celite and washed with MeOH/DCM (1/5, 50 mL×4). The filtrate was concentrated to give methyl 2-(3-(aminomethyl)phenyl)acetate hydrochloride BA2 (500 mg, crude) as a white solid. ¹H NMR (400 MHz, DMSO-d₆) δ_{H}=8.38 (s, 3H), 7.42-7.26 (m, 4H), 3.99 (s, 2H), 3.69 (s, 2H), 3.62 (s, 3H).

### Synthesis of 2-[3-((4-(2-(2-aminopyridin-3-yl)-5-phenyl-3H-imidazol4,5-b/pyridin-3-yl)benzamido)methyl)phenyl)acetic acid (Compound 52)

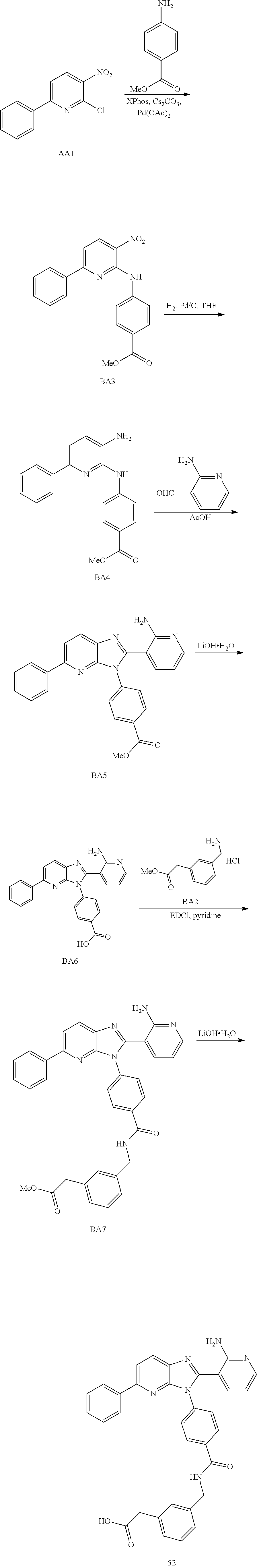

Step 1. To a solution of methyl 4-aminobenzoate (0,960 g, 6.39 mmol) in 1,4-dioxane (30.0 mL), 2-chloro-3-nitro-6-phenyl-pyridine AA1 (1.50 g, 6.39 mmol), Cs₂CO₃ (5.80 g, 17.9 mmol), XPhos (0.450 g, 0.950 mmol) and Pd(OAc)₂ (144 mg, 0.630 mmol) were added at 25° C., and the mixture stirred at 100° C. for 14 hrs. The reaction mixture was filtered, and filter cake washed with EtOAc (100 mL×3). Water (100 mL) was added, and the resulting suspension was extracted with EtOAc (200 mL). The combined organic phase was washed with brine (200 mL), dried over anhydrous Na₂SO₄, filtered and concentrated. The residue was purified by combi-flash column (EtOAC in PE, 5%) to give methyl 4-((3-nitro-6-phenylpyridin-2-yl)amino)benzoate BA3 (1.50 g, 49.5% yield) as a red solid. ¹H NMR (400 MHz, DMSO-d₆) δ_{H}=10.25 (s, 1H), 8.65 (d, J=8.8 Hz, 1H), 8.18-8.11 (m, 2H), 8.06-8.00 (m, 2H), 7.98-7.92 (m, 2H), 7.70 (d, J=8.8 Hz, 1H), 7.59-7.55 (m, 3H), 3.86 (s, 3H).

Step 2. To a solution of methyl 4-((3-nitro-6-phenylpyridin-2-yl)amino)benzoate BA3 (1.50 g, 4.29 mmol) in THF (20.0 mL), Pd/C (0.500 g, wet, 10%) was added and the reaction mixture was degassed and purged with Ar (×3). The reaction mixture was subsequently degassed and purged with H₂ (>3), and the mixture was stirred at 25° C. for 12 hrs under H₂ (15 Psi). The mixture was filtered through celite and washed with MeOH/DCM (1/5, 50 mL×4). The filtrate was concentrated to give methyl 4-((3-amino-6-phenylpyridin-2-yl)amino)benzoate BA4 (1.40 g, 56.1% yield) as a yellow oil. ¹H NMR (400 MHz, DMSO-d₆) δ_{H}=8.41 (s. 1H), 7.96-7.92 (m, 2H), 7.91-7.87 (m, 2H), 7.85-7.81 (m, 2H), 7.44-7.35 (m, 3H), 7.30-7.24 (m, 1H), 7.08-7.03 (m, 1H), 5.38 (s, 2H), 3.81 (s, 3H).

Step 3. To a mixture of methyl 4-((3-amino-6-phenylpyridin-2-yl)amino)benzoate BA4 (1.40 g, 4.01 mmol) in acetic acid (20.0 mL), 2-aminopyridine-3-carbaldehyde (0.590 g, 4.81 mmol) was added and the mixture was stirred at 100° C. for 3 hrs. The reaction mixture was concentrated directly, and the crude product was purified by combi-flash column (EtOAc in PE, 60%) to give methyl 4-(2-(2-aminopyridin-3-yl)-5-phenyl-3H-imidazo[4,5-b]pyridin-3-yl)benzoate BA5 (700 mg, 39.4% yield) as a yellow solid. ¹H NMR (400 MHz, DMSO-d₆) δ_{H}=8.29 (d, J=8.4 Hz, 1H), 8.15-8.10 (m, 2H), 8.08-7.99 (m, 4H), 7.69-7.63 (m, 2H), 7.50-7.44 (m, 2H), 7.43-7.37 (m, 1H), 7.24 (dd, J=2.0, 8.0 Hz, 1H), 6.87 (s, 2H), 6.46 (dd, J=4.8, 7.6 Hz, 1H), 3.90 (s, 3H).

Step 4. To a solution of methyl 4-(2-(2-aminopyridin-3-yl)-5-phenyl-3H-imidazo[4,5-b]pyridin-3-yl)benzoate BA5 (200 mg, 0.470 mmol) in THF (4.00 mL), methanol (2.00 mL) and water (4.00 mL), LiOH·H₂O (100 mg, 2.37 mmol) was added and the mixture was stirred at 25° C. for 2 hrs. The reaction mixture was diluted with H₂O (15 mL), and the resulting mixture was adjusted to pH ~3 by aq. HCl (2 M). The resulting mixture was extracted with EtOAc (30 mL×2), dried over anhydrous Na₂SO₄, filtered and concentrated to give 4-(2-(2-aminopyridin-3-yl)-5-phenyl-3H-imidazo[4,5-b]pyridin-3-yl)benzoic acid BA6 (200 mg, 93.1% yield) as a yellow solid. ¹H NMR (400 MHz, DMSO-d₆) δ_{H}=8.28 (d, J=8.4 Hz, 1H), 8.09-7.99 (m, 6H), 7.57 (d, J=8.4 Hz, 2H), 7.50-7.44 (m, 2H), 7.42-7.36 (m, 1H), 7.22 (dd, J=1.6, 7.6 Hz, 1H), 6.94 (s, 2H), 6.45 (dd, J=4.8, 7.6 Hz, 1H).

Step 5. To a solution of 4-(2-(2-aminopyridin-3-yl)-5-phenyl-3H-imidazo[4,5-b]pyridin-3-yl)benzoic acid BA6 (200 mg, 0.490 mmol) and methyl 2- (3-(aminomethyl)phenyl)acetate hydrochloride BA2 (88.0 mg, 0.490 mmol) in pyridine (5.00 mL), EDCI (141 mg, 0.730 mmol) was added and the mixture was stirred at 45° C. for 12 hrs. Water (20 mL) was added to the residue, and the resulting mixture was extracted with EtOAc (30 mL×3). The combined organic phase was washed with brine (30 mL×3), dried over anhydrous Na₂SO₄, filtered and concentrated. The residue was purified by flash silica gel chromatography (eluent with 0-5% of MeOH in DCM) to give methyl 2-(3-((4-(2-(2-aminopyridin-3-yl)-5-phenyl-3H-imidazo[4,5-b]pyridin-3-yl)benzamido)methyl)phenyl)acetate BA7 (200 mg, 65.4% yield) as a light yellow solid. ¹H NMR (400 MHz, DMSO-d₆) δ_{H}=9.22 (t, J=6.0 Hz, 1H), 8.29 (d, J=8.4 Hz, 1H), 8.09-7.99 (m, 6H), 7.65-7.60 (m, 2H), 7.50-7.43 (m, 2H), 7.42-7.37 (m, 1H), 7.33-7.22 (m, 4H), 7.18-7.14 (m, 1H), 6.92 (s, 2H), 6.45 (dd, J=4.8, 7.6 Hz, 1H), 4.50 (d, J=6.0 Hz, 2H), 3.68 (s, 2H), 3.61-3.59 (s, 3H).

Step 6. To a solution of methyl 2-(3-((4-(2-(2-aminopyridin-3-yl)-5-phenyl-3H-imidazo[4,5-b]pyridin-3-yl)benzamido)methyl)phenyl)acetate BA7 (200 mg, 0.350 mmol) in THF (2.00 mL) and water (2.00 mL). LiOH·H₂O (44.0 mg, 1.06 mmol) was added at 25° C., and the mixture was stirred for 2 hrs. HCl (2 M, 2.00 mL) was added and the mixture used directly for prep-HPLC (condition: water (FA)-ACN; Column: Xtimate C18 150*40 mm*10 μm; B %: 10%-50%; Gradient Time, 25 min) to give 2-(3-((4-(2-(2-aminopyridin-3-yl)-5-phenyl-3H-imidazo[4,5-b]pyridin-3-yl)benzamido)methyl)phenyl)acetic acid 52 (77.6 mg, 39.8% yield) as a white solid. ¹H NMR (400 MHz, DMSO-d₆) δ_{H}=9.22 (t, J=6.0 Hz, 1H), 8.29 (d, J=8.0 Hz, 1H), 8.08-7.99 (m, 6H), 7.62 (d, J=8.4 Hz, 2H), 7.50-7.44 (m, 2H), 7.42-7.36 (m, 1H), 7.31-7.21 (m, 4H), 7.15 (d, J=7.6 Hz, 1H), 6.92 (s, 2H), 6.46 (dd, J=4.8, 7.6 Hz, 1H), 4.49 (d, J=6.0 Hz, 2H), 3.55 (s, 2H). HPLC Rₜ=2.647 min in 8 min chromatography, purity 100%. LCMS Rₜ=1.759 min in 4 min chromatography, purity 99.8%, MS ESI calcd, for 554.21, [M+H]⁺ 555.21, found 555.2.

### Example 53. Synthesis of 4-[[4~ [2~ (2-amino-3-pyridyl)-5-phenyl-imidazo]4,5-b]pyridin-3-yl]phenyl]carbamoyl]benzoic acid (Compound 53)

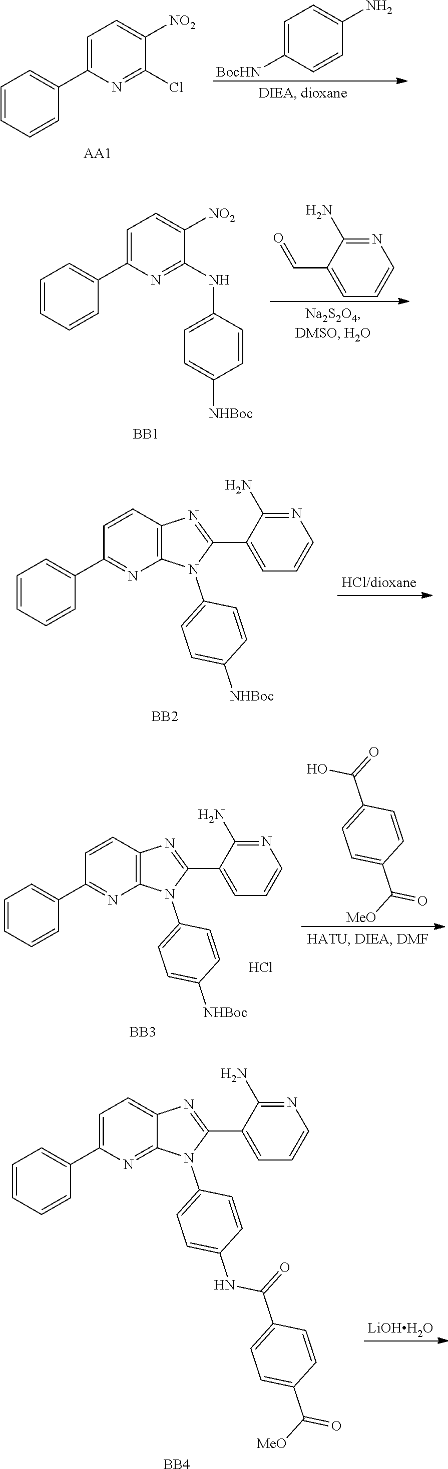

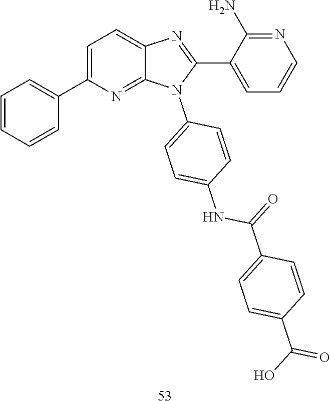 [SMILES: Nc1ncccc1-c1nc2ccc(-c3ccccc3)nc2n1-c1ccc(NC(=O)c2ccc(C(=O)O)cc2)cc1 ; "53"]

Step 1. To a solution of 2-chloro-3-nitro-6-phenyl-pyridine AA1 (444 mg, 2.13 mmol) in 1,4-dioxane (10.0 mL), DIEA (345 mg, 3.41 mmol) and tert-butyl (4-aminophenyl)carbamate (500 mg, 2.13 mmol) were added, and the mixture was stirred at 80° C. for 12 hrs. The reaction mixture was then diluted with water (50 mL) and extracted with ethyl acetate (200 mL×3). The organic layers were combined and washed with saturated aqueous NaHCO₃ (100 mL×2) solution, and brine (200 mL×5). The organic layers were dried over Na₂SO₄, filtered and concentrated in vacuo to afford tert-butyl (4-((3-nitro-6-phenylpyridin-2-yl)amino)phenyl)carbamate BB1 (800 mg, 92.3% yield) as a brown solid. ¹H NMR (400 MHz, DMSO-d₆) δ_{H}=10.05 (s, 1H), 9.39 (s, 1H), 8.59 (d, J=8.8 Hz, 1H), 8.13-8.03 (m, 2H), 7.66-7.46 (m, 8H), 1.49 (s, 9H).

Step 2. To a solution of tert-butyl (4-((3-nitro-6-phenylpyridin-2-yl)amino)phenyl)carbamate BB1 (700 mg, 1.72 mmol) and 2-aminopyridine-3-carbaldehyde (210 mg, 1.72 mmol) in DMSO (14.0 mL) and methanol (14.0 mL), Na₂S₂O₄ (0.750 g, 4.31 mmol) was added, and the mixture was stirred at 100° C. for 18 hrs. The reaction mixture was concentrated to dryness, then water (30 mL) was added and the resulting mixture was extracted with EtOAc (50 mL×3). The combined organic phase was washed with saturated LiCl aqueous (80 mL×3), dried over anhydrous Na₂SO₄, filtered and concentrated to dryness. The crude product was purified by combi-flash column (EtOAc in PE=0 to 41%) to afford tert-butyl (4-(2-(2-aminopyridin-3-yl)-5-phenyl-3H-imidazo[4,5-b]pyridin-3-yl)phenyl)carbamate BB2 (450 mg, 53.2% yield) as a yellow solid. ¹H NMR (400 MHz, DMSO-d₆) δ_{H}=9.65 (s, 1H), 8.25 (d, J=8.4 Hz, 1H), 8.10-7.94 (m, 4H), 7.62 (d, J=8.8 Hz, 2H), 7.50-7.43 (m, 2H), 7.42-7.35 (m, 3H), 7.23 (dd, J=2.0, 8.0 Hz, 1H), 7.06 (s, 2H), 6.43 (dd, J=4.8, 7.6 Hz, 1H), 1.50 (s, 9H).

Step 3. A solution of tert-butyl (4-(2-(2-aminopyridin-3-yl)-5-phenyl-3H-imidazo[4,5-b]pyridin-3-yl)phenyl)carbamate BB2 (500 mg, 1.04 mmol) in HCl/dioxane (6.00 mL, 4.0 M) was stirred at 25° C. for 2 hrs. The reaction mixture was concentrated directly to afford 3-(3-(4-aminophenyl)-5-phenyl-3H-imidazo[4,5-b]pyridin-2-yl)pyridin-2-amine hydrochloride BB3 (450 mg, crude) as a yellow oil. ¹H NMR (400 MHz, DMSO-d₆) δ_{H}=8.54-8.39 (m, 1H), 8.35 (d, J=8.4 Hz, 1H), 8.13 (dd, J=1.6, 6.0 Hz, 1H), 8.07-8.04 (m, 3H), 7.91 (dd, J=1.6, 7.6 Hz, 1H), 7.54-7.36 (m, 6H), 7.20-7.08 (m, 2H), 6.91 (dd, J=6.4, 7.2 Hz, 1H).

Step 4. To a stirred solution of 4-methoxycarbonylbenzoic acid (50.0 mg, 0.270 mmol) in DMF (5.00 mL), DIEA (170 mg, 1.32 mmol) and HATU (110 mg, 0.290 mmol) were added and the reaction mixture was stirred at 25° C. under N₂ for 20 mins. A solution of 3-(3-(4-aminophenyl)-5-phenyl-3H-imidazo[4,5-b]pyridin-2-yl)pyridin-2-amine hydrochloride BB3 (100 mg, 0.260 mmol) in DMF (5.00 mL) was then added to the reaction mixture dropwise and the resulting mixture was stirred at 25° C. under N₂ for 2 hrs. The reaction mixture was diluted with EtOAc (20 mL), washed with water (20 mL) and brine (50 mL), dried over anhydrous sodium sulfate, filtered and the filtrate was concentrated. The crude product was purified by flash chromatography on silica gel (95% of EtOAc in PE) to afford methyl 4-[[4-[2-(2-amino-3-pyridyl)-5-phenyl-imidazo[4,5-b]pyridin-3-yl]phenyl]carbamoyl]benzoate BB4 (50.0 mg, 35.0% yield) as a yellow solid. LCMS Rₜ=0.530 min in 1.0 min chromatography, purity 77.2%, MS ESI calcd, for 540.19 [M+H]⁺ 541.19, found 541.2.

Step 5. To a stirred solution of methyl 4-[[4-[2-(2-amino-3-pyridyl)-5-phenyl-imidazo[4,5-b]pyridin-3-yl]phenyl]carbamoyl]benzoate BB4 (50.0 mg, 0.0900 mmol) in THE (2.00 mL) and water (2.00 mL), LiOH·H₂O (7.40 mg, 0.180 mmol) was added and the reaction was stirred at 25° C. for 12 hrs. The reaction mixture was concentrated and diluted with DMSO (2.0 mL). The mixture was adjusted to pH ~7 with 1 M HCl and purified by prep-HPLC (column: Welch Xtimate C18 150*30 mm*5 μm, method: water (NH₃H₂O)+NHλHCO₃)-ACN, begin B: 24, end B: 54) to afford 4-[[4-[2-(2-amino-3-pyridyl)-5-phenyl-imidazo[4,5-b[pyridin-3-yl]phenyl]carbamoyl]benzoic acid 53 (15.0 mg, 30.4% yield) as a yellow solid. ¹H NMR (400 MHz, DMSO-d₆) δ=10.64 (s, 1H), 8.27 (d, J=8.4 Hz, 1H), 8.16-7.90 (m, 10H), 7.54-7.44 (m, 4H), 7.40 (d, J=7.2 Hz, 1H), 7.28 (dd, J=2.0, 7.6 Hz, 1H), 7.04 (s, 2H), 6.46 (dd, J=4.8, 7.6 Hz, 1H). HPLC Rₜ=1.944 min in 8 min chromatography, purity 97.5%. LCMS Rₜ=0.974 min in 3 min chromatography, purity 100%, MS ESI calcd, for 526.18 [M+H]⁺ 527.18, found 527.2.

### Example 54. Synthesis of 3-[[4-[2-(2-amino-3-pyridyl)-5-phenyl-imidazo[4,5-b]pyridin-3-yl]phenyl]carbamoyl]benzoic acid (Compound 54)

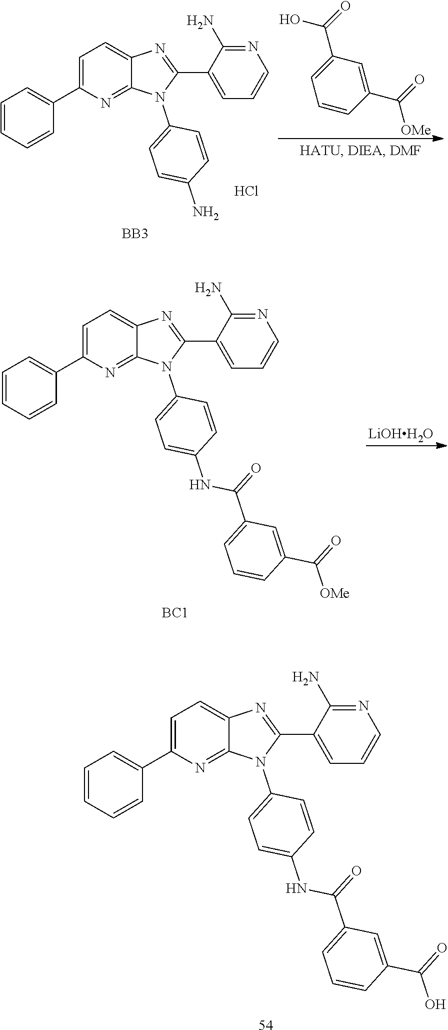

Step 1. To a stirred solution of 3-methoxycarbonylbenzoic acid (48.0 mg, 0.260 mmol) in DMF (5.00 mL), DIEA (0.240 mL, 1.32 mmol) and HATU (110 mg, 0.290 mmol) were added, and the reaction mixture was stirred at 25° C. under N₂ for 20 mins. A solution of 3-[3-(4-aminophenyl)-5-phenyl-imidazo[4,5-b]pyridin-2-yl]pyridin-2-amine hydrochloride BB3 (100 mg, 0.260 mmol) in DMF (5.00 mL) was then added to the reaction mixture dropwise and the resulting mixture stirred at 25° C. under N₂ for 2 hrs. The reaction mixture was diluted with EtOAc (20 mL), washed with water (20 mL) and brine (50 mL), dried over anhydrous sodium sulfate, filtered and the filtrate was concentrated. The crude product was purified by flash chromatography on silica gel (95% of EtOAc in PE) to afford methyl 3-[[4-[2-(2-amino-3-pyridyl)-5-phenyl-imidazo[4,5-b]pyridin-3-yl]phenyl]carbamoyl]benzoate BC1 (50.0 mg, 35.0% yield) as a yellow solid. ¹H NMR (400 MHz, DMSO-d₆) δ=10.71 (s, 1H), 8.32-8.23 (m, 2H), 8.19 (d, J=7.6 Hz, 1H), 8.08-8.03 (m, 2H), 8.03-8.00 (m, 2H), 7.99-7.94 (m, 3H), 7.73 (t, J=7.6 Hz, 1H), 7.53-7.36 (m, 6H), 7.28 (dd, J=1.6, 7.6 Hz, 1H), 7.04 (br d, J=5.2 Hz, 1H), 6.46 (dd, J=4.8, 7.6 Hz, 1H), 3.93 (s, 3H).

Step 2. To a stirred solution of methyl 3-[[4-[2-(2-amino-3-pyridyl)-5-phenyl-imidazo[4,5-b]pyridin-3-yl]phenyl]carbamoyl]benzoate BC1 (50.0 mg, 0.0900 mmol) in THE (2.00 mL) and water (2.00 mL), LiOH·H₂O (7.40 mg, 0.190 mmol) was added and the reaction stirred at 25° C. for 12 hrs. The reaction mixture was concentrated and diluted with DMSO (2.0 mL). The mixture was adjusted to pH ~7 with 1 M HCl and purified by prep-HPLC (column: welch xtimate C18 150*30 mm*5 μm, method: water (NH₄HCO₃+NH₃H₂O)-ACN, begin B: 24, end B: 54) to afford 3-[[4-[2-(2-amino-3-pyridyl)-5-phenyl-imidazo[4,5-b]pyridin-3-yl]phenyl]carbamoyl]benzoic acid 54 (19.0 mg, 38.4% yield) as a yellow solid. ¹H NMR (400 MHz, DMSO-d₆) δ=10.68 (s, 1H), 8.55 (s, 1H), 8.27 (d, J=8.4 Hz, 1H), 8.18 (dd, J=7.6, 18.4 Hz, 2H), 8.09-7.94 (m, 6H), 7.69 (t, J=7.6 Hz, 1H), 7.55-7.44 (m, 4H), 7.40 (d, J=7.2 Hz, 1H), 7.28 (dd, J=1.6, 7.6 Hz, TH), 7.04 (s, 2H), 6.46 (dd, J=4.8, 7.6 Hz, 1H). HPLC Rₜ=5.215 min in 8 min chromatography, purity 98.8%. LCMS Rₜ=1.01 min in 3 min chromatography, purity 99.8%, MS ESI calcd, for 526.18 [M+H]⁺ 527.18, found 527.2.

### Example 55. Synthesis of 3-[4-[2-(2-amino-3-pyridyl)-5-phenyl-imidazo[4,5-b]pyridin-3-yl]phenyl]carbamoyl]-2-fluoro-benzoic acid (Compound 55)

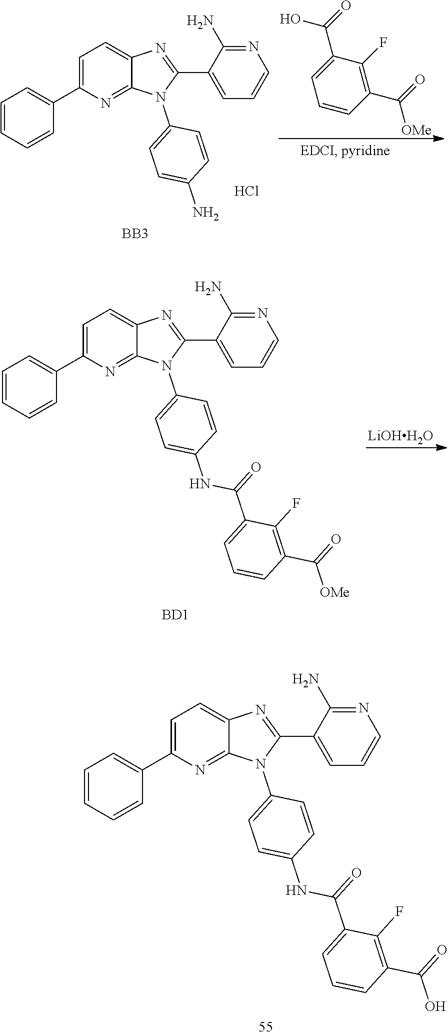

Step 1. To the solution of 2-fluoro-3-methoxycarbonyl-benzoic acid (100 mg, 0.510 mmol) and 3-[3-(4-aminophenyl)-5-phenyl-imidazo[4,5-b]pyridin-2-yl]pyridin-2-amine hydrochloride BB3 (191 mg, 0.510 mmol) in pyridine (2.00 mL), EDCI (230 mg, 0.610 mmol) was added and the reaction was stirred at 25° C. under N₂ for 12 hrs. The reaction mixture was diluted with EtOAc (20 mL), washed with water (20 mL), 1 M HCl (20 mL×2) and brine (20 mL). The combined organic layers were dried over anhydrous sodium sulfate, filtered and the filtrate was concentrated. The crude product was purified by prep-HPLC (column: welch xtimate C18 150*30 mm*5 μm, method: water (NH₄HCO₃+NH₃H₂O)-ACN, begin B: 53, end B: 83) to afford methyl 3-[[4-[2-(2-amino-3-pyridyl)-5-phenyl-imidazo[4,5-b]pyridin-3-yl]phenyl]carbamoyl]-2-fluoro-benzoate BD1 (30.0 mg, 10.6% yield) as a yellow solid. ¹H NMR (400 MHz, DMSO-d₆) δ=10.83 (s, 1H), 8.27 (d, J=8.4 Hz, 1H), 8.11-7.80 (m, 8H), 7.59-7.36 (m, 6H), 7.28 (dd, J=1.6, 7.6 Hz, 1H), 7.02 (s, 2H), 6.47 (dd, J=4.8, 7.6 Hz, 1H), 3.91 (s, 3H).

Step 2. To a stirred solution of methyl 3-[[4-[2-(2-amino-3-pyridyl)-5-phenyl-imidazo[4,5-b]pyridin-3-yl]phenyl]carbamoyl]-2-fluoro-benzoate BD1 (30.0 mg, 0.0500 mmol) in THF (1.00 mL) and water (1.00 mL), LiOH·H₂O (4.30 mg, 0.110 mmol) was added and the reaction mixture was stirred at 25° C. under N₂ for 16 hrs. The reaction mixture was adjusted to pH ~7 with 1 M HCl solution and concentrated to afford a residue. The crude product was purified by prep-HPLC (column: welch xtimate c18 150*30 mm*5 μm, method: water (NH₄HCO₃+NH₃H₂O)-ACN, begin B: 24, end B: 54) to afford 3-[[4-[2-(2-amino-3-pyridyl)-5-phenyl-imidazo[4,5-b]pyridin-3-yl]phenyl]carbamoyl]-2-fluoro-benzoic acid 55 (24.0 mg, 79.1% yield) as a yellow solid. ¹H NMR (400 MHz, DMSO-d₆) δ=10.79 (s, 1H), 8.27 (d, J=8.4 Hz, 1H), 8.13-7.75 (m, 8H), 7.57-7.35 (m, 6H), 7.28 (dd, J=1.6, 7.6 Hz, 1H), 7.03 (s, 2H), 6.48 (dd, J=4.8, 7.6 Hz, 1H). 1° F. NMR (400 MHz, DMSO-d₆) δ=−114.301. HPLC Rₜ=5.175 min in 8 min chromatography, purity 98.4%. LCMS Rₜ=2.698 min in 4 min chromatography, purity 100%, MS ESI calcd, for 544.17 [M+H]⁺ 545.17, found 545.1.

### Example 56. Synthesis of 2-(3-((4-(2-(2-aminopyridin-3-yl)-5-phenyl-3H-imidazo[4,5-b]pyridin-3-yl)phenyl)carbamoyl)phenyl)acetic acid (Compound 56)

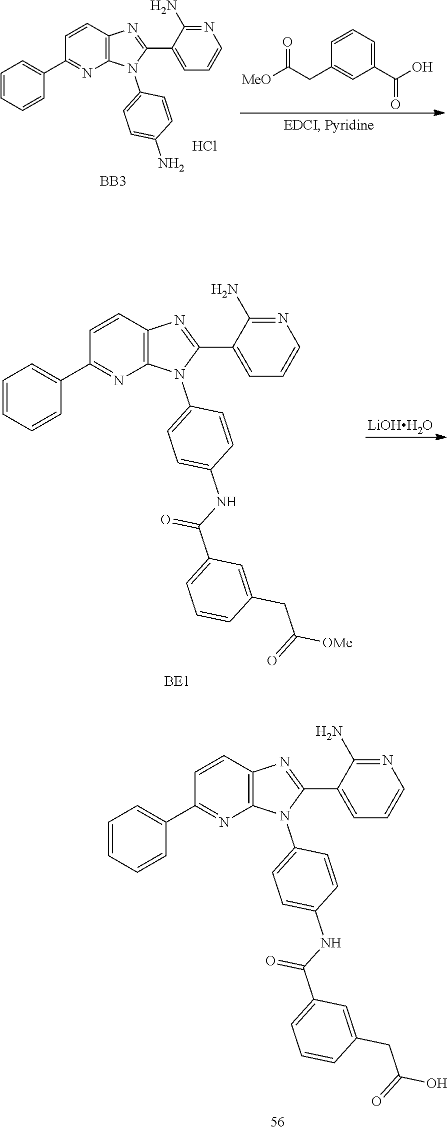

Step 1. To a solution of 3-(3-(4-aminophenyl)-5-phenyl-3H-imidazo[4,5-b]pyridin-2-yl)pyridin-2-amine hydrochloride BB3 (150 mg, 0.390 mmol) in pyridine (5.00 mL), 3-(2-methoxy-2-oxo-ethyl)benzoic acid (77.0 mg, 0.390 mmol) and EDCI (114 mg, 0.590 mmol) were added and the mixture was stirred at 25° C. for 2 hrs. The reaction mixture was poured into water (30 mL), and the resulting mixture was extracted with EtOAc (20 mL×2). The combined organic phase was washed with HCl aqueous (1 M, 30 mL×2) and brine (30 mL), dried over anhydrous Na₂SO₄, filtered and concentrated to dryness. The crude product was purified by flash column (EtOAc in PE=0 to 66%) to afford methyl 2-(3-((4-(2-(2-aminopyridin-3-yl)-5-phenyl-3H-imidazo[4,5-b]pyridin-3-yl)phenyl)carbamoyl)phenyl)acetate BE1 (140 mg, 62.6% yield) as a yellow solid. ¹H NMR (400 MHz, DMSO-d₆) δ_{H}=10.50 (s, 1H), 8.27 (d, J=8.4 Hz, 1H), 8.08-7.98 (m, 4H), 7.97-7.92 (m, 2H), 7.90-7.85 (m, 2H), 7.53-7.45 (m, 6H), 7.42-7.37 (m, 1H), 7.28 (dd, J=1.6, 7.6 Hz, 1H), 7.04 (s, 2H), 6.46 (dd, J=5.2, 7.6 Hz, 1H), 3.82 (s, 2H), 3.65 (s, 3H).

Step 2. To a solution of methyl 2-(3-((4-(2-(2-aminopyridin-3-yl)-5-phenyl-3H-imidazo[4,5-b]pyridin-3-yl)phenyl)carbamoyl)phenyl)acetate BE1 (140 mg, 0.250 mmol) in THF (4.00 mL) and ater (2.00 mL), LiOH·H₂O (53.0 mg, 1.26 mmol) was added and the mixture was stirred at 25° C. for 2 hrs. The reaction mixture was diluted with water (2 mL) and purified by prep-HPLC (column: welch xtimate c18 150*40 mm*10 μm; mobile phase: [water (HCl)-ACN]: B %: 10%-50%: 36 min) to afford 2-(3-((4-(2-(2-aminopyridin-3-yl)-5-phenyl-3H-imidazo[4,5-b]pyridin-3-yl)phenyl)carbamoyl)phenyl)acetic acid 56 (60.5 mg, 43.7% yield) as a yellow solid. ¹H NMR (400 MHz, DMSO-d₆) δH=10.56 (s, 1H), 8.66-8.25 (m, 3H), 8.13 (dd, J=1.6, 6.4 Hz, 1H), 8.10-8.03 (m, 3H), 8.02-7.97 (m, 2H), 7.93-7.85 (m, 3H), 7.60-7.54 (m, 2H), 7.53-7.46 (m, 4H), 7.45-7.39 (m, 1H), 6.92 (dd, J=6.0, 7.2 Hz, 1H), 3.71 (s, 2H). HPLC Rₜ=2.567 min in 8 min chromatography, purity 98.7%. LCMS Rₜ=3.072 min in 4 min; Agilent PoroShell 120 EC-C18 2.7 μm 3.0*50 mm; purity 97.1%, MS ESI calcd, for 540.19, [M+H]⁺ 541.19, found 541.2.

### Example 57. Synthesis of 3-(3-((4-(2-(2-aminopyridin-3-yl)-5-phenyl-3H-imidazo[4,5-b]pyridin-3-yl)phenyl)carbamoyl)phenyl) propanoic acid (Compound 57)

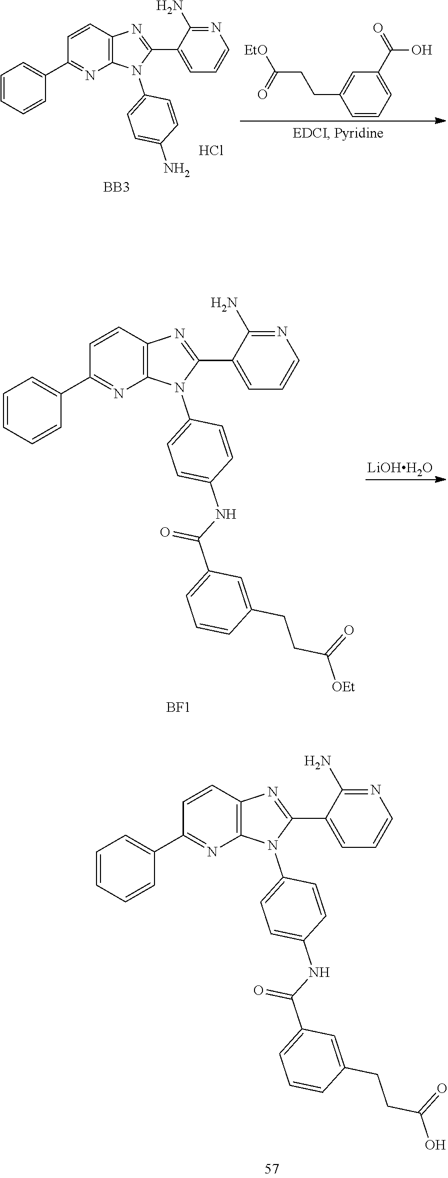

Step 1. To a solution of 3-(3-(4-aminophenyl)-5-phenyl-3H-imidazo[4,5-b]pyridin-2-yl)pyridin-2-amine hydrochloride BB3 (150 mg, 0.390 mmol) in pyridine (5.00 mL), 3-(3-ethoxy-3-oxo-propyl)benzoic acid (88.0 mg, 0.390 mmol) and EDCI (114 mg, 0.590 mmol) were added and the mixture was stirred at 25° C. for 2 hrs. The reaction mixture was poured into water (30 mL) and the resulting mixture was extracted with EtOAc (20 mL×2). The combined organic phase was washed with HCl aqueous (1 M, 30 mL×2), brine (30 mL), dried over anhydrous Na₂SO₄, filtered and concentrated to dryness. The crude product was purified by flash column (EtOAc in PE=65%) to give ethyl 3-(3-((4-(2-(2-aminopyridin-3-yl)-5-phenyl-3H-imidazo[4,5-b]pyridin-3-yl)phenyl)carbamoyl)phenyl) propanoate BF1 (200 mg, 82.5% yield) as a yellow solid. ¹H NMR (400 MHz, DMSO-d₆) δ_{H}=10.44 (s, 1H), 8.26 (d, J=8.4 Hz, 1H), 8.08-7.98 (m, 4H), 7.97-7.92 (m, 2H), 7.86-7.77 (m, 2H), 7.51-7.44 (m, 6H), 7.42-7.37 (m, 1H), 7.28 (dd, J=2.0, 7.6 Hz, 1H), 7.05 (s, 2H), 6.46 (dd, J=4.8, 7.6 Hz, 1H), 4.05 (q, J=7.2 Hz, 2H), 2.99-2.93 (m, 2H), 2.73-2.67 (m, 2H), 1.16 (1, J=7.2 Hz, 3H).

Step 2. To a solution of ethyl 3-(3-((4-(2-(2-aminopyridin-3-yl)-5-phenyl-3H-imidazo[4,5-b]pyridin-3-yl)phenyl)carbamoyl)phenyl) propanoate BF1 (200 mg, 0.350 mmol) in THE (4.00 mL) and water (2.00 mL), LiOH·H₂O (74.0 mg, 1.76 mmol) was added and the mixture was stirred at 25° C. for 2 hrs. The reaction mixture was diluted with water (2 mL) and the crude product purified by prep-HPLC (column: welch xtimate c18 150*40 mm*10 μm; mobile phase: [water (HCl)-ACN]: B %: 12%-52%: 36 min) to afford 3-(3-(4-(2-(2-aminopyridin-3-yl)-5-phenyl-3H-imidazo[4,5-b]pyridin-3-yl)phenyl)carbamoyl)phenyl) propanoic acid 57 (44.0 mg, 22.2% yield) as a yellow solid. ¹H NMR (400 MHz, DMSO-d₆) δ_{H}=10.49 (s, 1H), 8.35 (d, J=8.0 Hz, 1H), 8.30-8.13 (m, 1H), 8.12-8.03 (m, 4H), 8.00-7.94 (m, 2H), 7.86-7.76 (m, 3H), 7.60-7.52 (m, 2H), 7.52-7.39 (m, 5H), 6.90-6.80 (m, 1H), 2.97-2.89 (m, 2H), 2.65-2.59 (m, 2H). HPLC Rₜ=4.383 min in 8 min chromatography, purity 98.8%. LCMS Rₜ=3.111 min in 4 min; Agilent PoroShell 120 EC-C18 2.7 μm 3.0*50 mm; purity 99.1%, MS ESI calcd, for 554.21, [M+H]⁺ 555.21, found 555.2.

### Example 58. Synthesis of 4-[[4-[2-(2-amino-3-pyridyl)-5-phenyl-imidazo[4,5-b]pyridin-3-yl]anilino]methyl]benzoic acid (Compound 58)

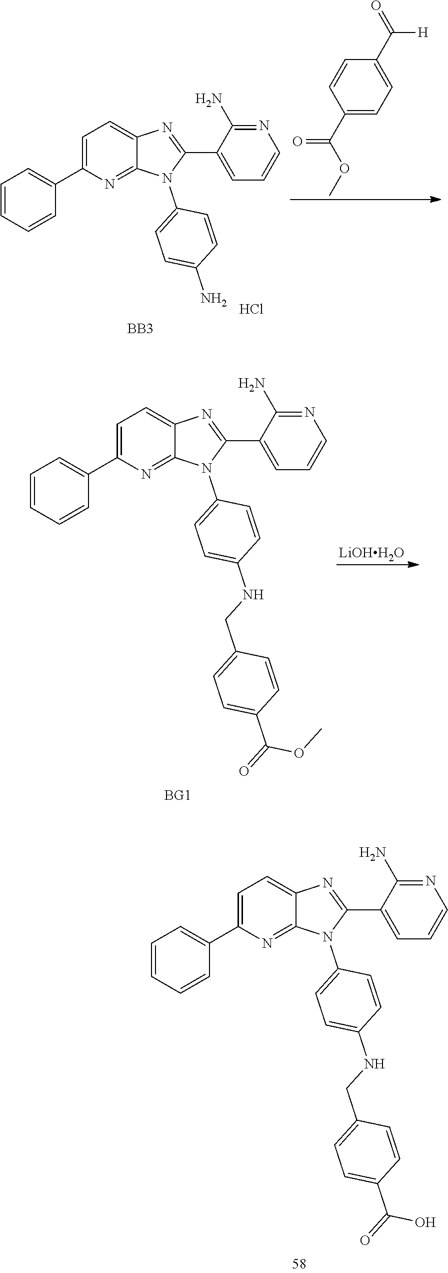

Step 1. To a stirred solution of 3-[3-(4-aminophenyl)-5-phenyl-imidazo[4,5-b[pyridin-2-yl]pyridin-2-amine hydrochloride BB3 (30.0 mg, 0.0200 mmol) in DCM (3.00 mL), acetic acid (32.0 mg, 0.320 mmol) and methyl 4-formylbenzoate (15.0 mg, 1.15 mmol) were added and the mixture was stirred at 25° C., under N₂, for 16 hrs. Sodium triacetoxyborohydride (34.0 mg, 0.160 mmol) was then added, dropwise, and the resulting mixture was stirred at 25° C. under N₂ for 10 hrs. The reaction mixture was quenched with water (10 mL) and extracted with EtOAc (15 mL×2), the combined organic layers were washed with brine (20 mL), dried over anhydrous sodium sulfate, filtered and the filtrate was concentrated to afford methyl 4-[[4-[2-(2-amino-3-pyridyl)-5-phenyl-imidazo[4,5-b]pyridin-3-yl]anilino]methyl]benzoate BG1 (30.0 mg, 71.8% yield) as a yellow oil. LCMS Rₜ=0.55 min in 1.0 min chromatography, purity 73.4%, MS ESI calcd, for 526.21 [M+H]⁺ 527.21, found 527.3.

Step 2. To a stirred solution of methyl 4-[[4-[2-(2-amino-3-pyridyl)-5-phenyl-imidazo[4,5-b]pyridin-3-yl]anilino]methyl]benzoate BG1 (30.0 mg, 0.0600 mmol) in THF (1.00 mL) and water (1.00 mL), LiOH·H₂O (4.60 mg, 0.110 mmol) was added and the reaction mixture was stirred at 25° C. under N₂ for 16 hrs. The reaction mixture was adjusted to pH ~5 with 1 M HCl and concentrated. The crude product was purified by prep-HPLC (column: welch xtimate C18 150×30 mm×5 μm, method: water (FA)-ACN, begin B: 26, end B: 56) to afford 4-[[4-[2-(2-amino-3-pyridyl)-5~phenyl-imidazo[4,5-b]pyridin-3-yl]anilino]methyl]benzoic acid 58 (4.20 mg, 14.3% yield) as a yellow solid. ¹H NMR (400 MHz, DMSO-d₆) δ=12.81 (s, 1H), 8.21 (d, J=8.4 Hz, 1H), 8.05-7.86 (m, 6H), 7.59-7.33 (m, 5H), 7.27-7.07 (m, 5H), 6.76-6.60 (m, 3H), 6.40 (dd, J=4.8, 7.6 Hz, 1H), 4.41 (d, J=6.0 Hz, 2H). HPLC Rₜ=5.390 min in 8 min chromatography, purity 98.4%. LCMS Rₜ=2.238 min in 4 min chromatography, purity 99.1%, MS ESI calcd, for 512.20 [M+H]⁺ 513.20, found 513.0.

### Example 59. Synthesis of 4-((5-(2-(2-aminopyridin-3-yl)-5-phenyl-3H-imidazo[4,5-b]pyridin-3-yl)pyridin-2-yl)carbamoyl)benzoic acid (Compound 59)

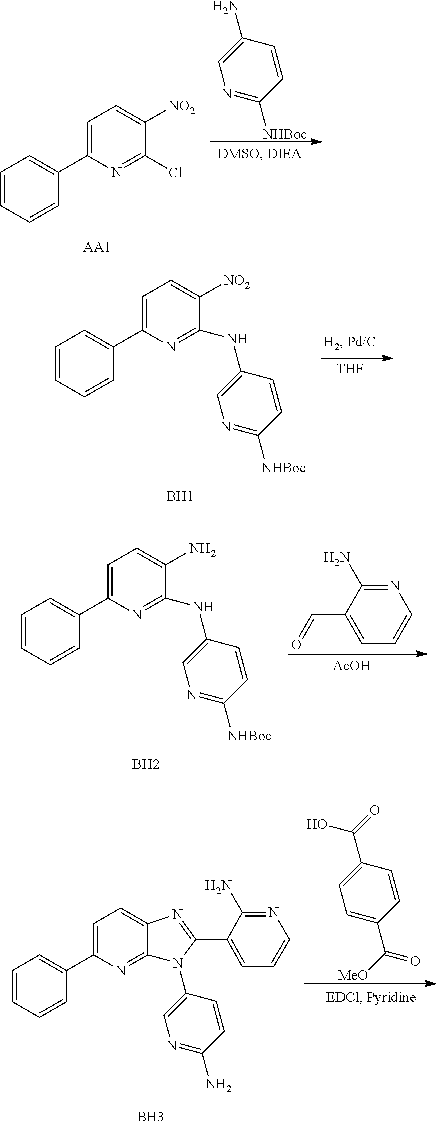

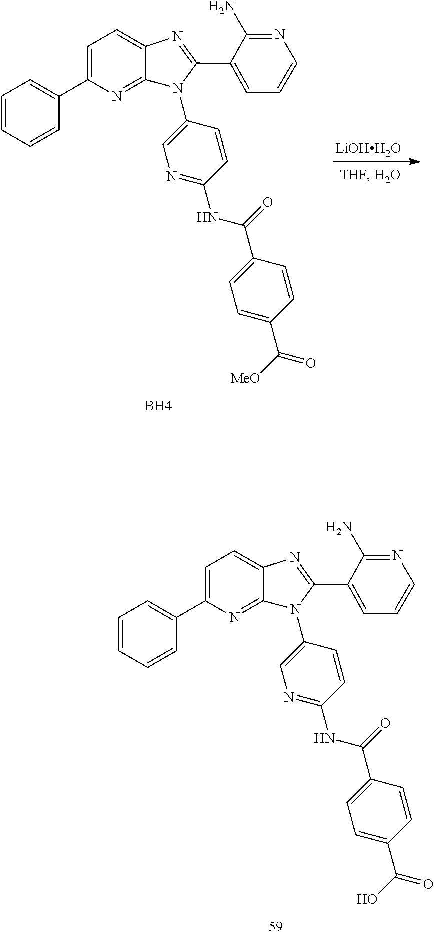

Step 1. To a solution of 2-chloro-3-nitro-6-phenyl-pyridine AA1 (1.70 g, 7.25 mmol) in DMSO (15.0 mL), tert-butyl (5-aminopyridin-2-yl)carbamate (1.82 g, 8.69 mmol) and DIEA (2.81 g, 21.7 mmol) were added at 25° C., and the mixture was stirred at 80° C. for 14 hrs. EtOAc (20 mL) was added and the resulting suspension was washed with saturated aqueous LiCl (20 mL×3), dried over anhydrous Na₂SO₄, filtered and concentrated. The mixture was triturated with isopropyl ether (20 mL) for 10 mins and filtered. The filter cake was concentrated to give tert-butyl (5-((3-nitro-6-phenylpyridin-2-yl)amino)pyridin-2-yl)carbamate BH1 (3.80 g, crude) as a red solid. ¹H NMR (CDCl₃, 400 MHz) δu=10.18 (s, 1H), 8.65-8.55 (m, 2H), 8.18 (d, J=9.2 Hz, 1H), 8.09-7.98 (m, 3H), 7.66-7.58 (m, 1H), 7.52 (s, 3H), 7.36 (d, J=8.4 Hz, 1H), 1.57 (s, 9H).

Step 2. To a mixture of tert-butyl (5-((3-nitro-6-phenylpyridin-2-yl)amino)pyridin-2-yl)carbamate BH1 (2.50 g, 9.68 mmol) in THF (20.0 mL), wet Pd/C (500 mg, 10%) was added under an Ar atmosphere. The suspension was degassed and purged with H₂ (×3) and the mixture was stirred under H₂ (15 Psi) at 25° C. for 14 hrs. The mixture was filtered via a celite pad, and the pad washed with DCM/MeOH (1/1, 150 mL×5). The filtrate was concentrated to give tert-butyl (5-((3-amino-6-phenylpyridin-2-yl)amino)pyridin-2-yl)carbamate BH2 (2.00 g, 79.4% yield) as a yellow solid, which was used in the next step without further purification. ¹H NMR (DMSO-d₆, 400 MHz) δ_{H}=9.53 (s, 1H), 8.74 (d, J=2.4 Hz, 1H), 8.10 (dd, J=2.8, 9.2 Hz, 1H), 7.98 (s, 1H), 7.90 (dd, J=1, 6, 8.8 Hz, 2H), 7.73 (d, J=9.2 Hz, 1H), 7.40 (t, J=7.6 Hz, 2H), 7.29-7.22 (m, 2H), 6.98 (d, J=7.6 Hz, 1H), 5.28 (s, 2H), 1.48 (s, 9H).

Step 3. To a solution of ter-butyl (5-((3-amino-6-phenylpyridin-2-yl)amino)pyridin-2-yl)carbamate BH2 (2.00 g, 5.30 mmol) in acetic acid (20.0 mL), 2-aminopyridine-3-carbaldehyde (777 mg, 6.36 mmol) was added at 25° C., and the mixture was stirred at 100° C. for 24 hrs. The reaction was concentrated, and the resulting residue was purified by flash column (EtOAc in PE=0%-100%) to give 3-(3-(6-aminopyridin-3-yl)-5-phenyl-3H-imidazo[4,5-b]pyridin-2-yl)pyridin-2-amine BH3 (320 mg, 15.9% yield) as a yellow solid. LCMS Rₜ=0.938 min in 2.5 min chromatography, purity 60.3%, MS ESI calcd. for 379.15. [M+H]⁺ 380.15, found 380.1.

Step 4. To a mixture of 4-(methoxycarbonyl)benzoic acid (51.0 mg, 0.260 mmol) and 3-(3-(6-aminopyridin-3-yl)-5-phenyl-3H-imidazo[4,5-b]pyridin-2-yl)pyridin-2-amine BH3 (100 mg, 0.260 mmol) in pyridine (2.00 mL), EDCI (152 mg, 0.790 mmol) was added and the mixture was stirred at 80° C. for 2 hrs. Water (30 mL) was added into the mixture, and the resulting mixture was extracted with EtOAc (20 mL×3). The combined organic phase was dried over anhydrous Na₂SO₄, filtered and concentrated. The residue was purified by flash column (EtOAc in PE=0%-62%) to give methyl 4-((5-(2-(2-aminopyridin-3-yl)-5-phenyl-3H-imidazo[4,5-b]pyridin-3-yl)pyridin-2-yl)carbamoyl)benzoate BI4 (30.0 mg, 20.5% yield) as a yellow solid. LCMS RE 1.348 min in 4 min chromatography, purity 55.9%, MS ESI calcd. for 541.19, [M+H]⁺ 542.19, found 542.0.

Step 5. To a solution of methyl 4-((5-(2-(2-aminopyridin-3-yl)-5-phenyl-3H-imidazo[4,5-b]pyridin-3-yl)pyridin-2-yl)carbamoyl)benzoate BH4 (30.0 mg, 0.0500 mmol) in THF (1.00 mL) and water (1.00 mL), LiOH·H₂O (3.50 mg, 0.0800 mmol) was added at 25° C. and the mixture was stirred at 25° C. for 2 hrs. HCl (2 M, 1.00 mL) was added and the mixture purified by prep-HPLC (condition: water (FA)-ACN; Column: Xtimate C18 150*40 mm*10 μm; B %; 8%-48%; Gradient Time: 25 min) to afford 4-((5-(2-(2-aminopyridin-3-yl)-5-phenyl-3H-imidazo[4,5-b]pyridin-3-yl)pyridin-2-yl)carbamoyl)benzoic acid 59 (10.0 mg, 34.0% yield) as a white solid. ¹H NMR (DMSO-d₆, 400 MHz) δ_{H}=11.23 (s, 1H), 8.54 (d, J=2.8 Hz, 1H), 8.38 (d, J=9.2 Hz, 1H), 8.31 (d, J=8.4 Hz, 1H), 8.16-8.01 (m, 9H), 7.52-7.46 (m. 2H), 7.44-7.39 (m, 2H), 6.84 (s, 2H), 6.54 (dd, J=4.8, 7.6 Hz, 1H). HPLC Rₜ 2.794 min in 8 min chromatography, purity 99.4%. LCMS Rₜ=1.879 min in 4 min chromatography, purity 99.9%, MS ESI calcd, for 527.17, [M+H]⁺ 528.17, found 528.2.

### Example 60. Synthesis of 3-((5-(2-(2-aminopyridin-3-yl)-5-phenyl-3H-imidazo[4,5-b]pyridin-3-yl)pyridin-2-yl)carbamoyl)benzoic acid (Compound 60)

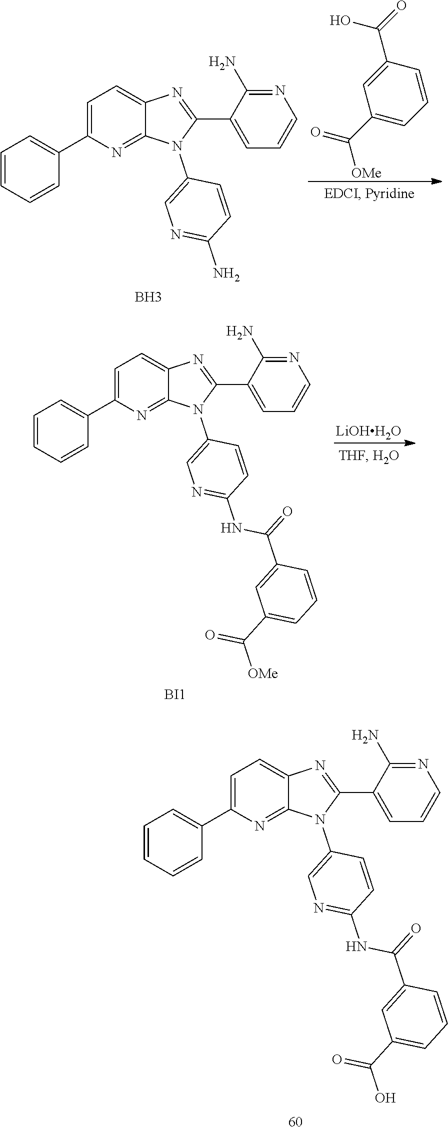

Step 1. To a solution of 3-(3-(6-aminopyridin-3-yl)-5-phenyl-3H-imidazo[4,5-b]pyridin-2-yl)pyridin-2-amine BH3 (110 mg, 0.290 mmol) in pyridine (2.00 mL), 3-(methoxycarbonyl)benzoic acid (63.0 mg, 0.340 mmol) and EDCI (124 mg, 0.870 mmol) were added at 25° C., and the mixture was stirred at 80° C. for 2 hrs. Water (10 mL) was added and the resulting mixture was extracted with EtOAc (20 mL×3). The combined organic phase was dried over anhydrous Na₂SO₄, filtered and concentrated. The residue was purified by flash column (EtOAc in PE=0%-62%) to give methyl 3-((5-(2-(2-aminopyridin-3-yl)-5-phenyl-3H-imidazo[4,5-b]pyridin-3-yl)pyridin-2-yl)carbamoyl)benzoate BI1 (30.0 mg, 19.0% yield) as a white solid. LCMS Rₜ=1.400 min in 2.5 min chromatography, purity 53.4%, MS ESI calcd. for 541.19, [M+H]⁺ 542.19, found 542.3.

Step 2. To a solution of methyl 3-((5-(2-(2-aminopyridin-3-yl)-5-phenyl-3H-imidazo[4,5-b]pyridin-3-yl)pyridin-2-yl)carbamoyl)benzoate BI1 (30.0 mg, 0.0500 mmol) in THF (1.00 mL) and water (1.00 mL), LiOH·H₂O (3.50 mg, 0.0800 mmol) was added at 25° C., and the mixture was stirred for 2 hrs. HCl (2 M, 1 mL) was added and the mixture was purified by prep-HPLC (condition: water (FA)-ACN; Column: Xtimate C18 150*40 mm*10 μm; B %: 8%-48%; Gradient Time, 25 min) to afford 3-((5-(2-(2-aminopyridin-3-yl)-5-phenyl-3H-imidazo[4,5-b]pyridin-3-yl)pyridin-2-yl)carbamoyl)benzoic acid 60 (10.1 mg, 34.1% yield) as a white solid. ¹H NMR (DMSO-d₆, 400 MHz) δ_{H}=13.42-13.12 (s, 1H), 11.30 (s, 1H), 8.58 (t, J=1.6 Hz, 1H), 8.54 (d, J=2.4 Hz, 1H), 8.37 (d, J=9.2 Hz, 1H), 8.33-8.26 (m, 2H), 8.18-8.14 (m, 1H), 8.10-8.02 (m, 5H), 7.68 (t, J=7.6 Hz, 1H), 7.52-7.46 (m, 2H), 7.44-7.38 (m, 2H), 6.84 (s, 2H), 6.54 (dd, J=4.8, 7.6 Hz, 1H). HPLC Re 2.689 min in 8 min chromatography, purity 98.6%. LCMS R (=1.713 min in 4 min chromatography, purity 98.3%, MS ESI calcd, for 527.17, [M+H]⁺ 528.17, found 528.1.

### Example 61. Synthesis of 3-((5-(2-(2-aminopyridin-3-yl)-5-phenyl-3H-imidazo[4,5-b]pyridin-3-yl)pyridin-2-yl)carbamoyl)-2-fluorobenzoic acid (Compound 61)

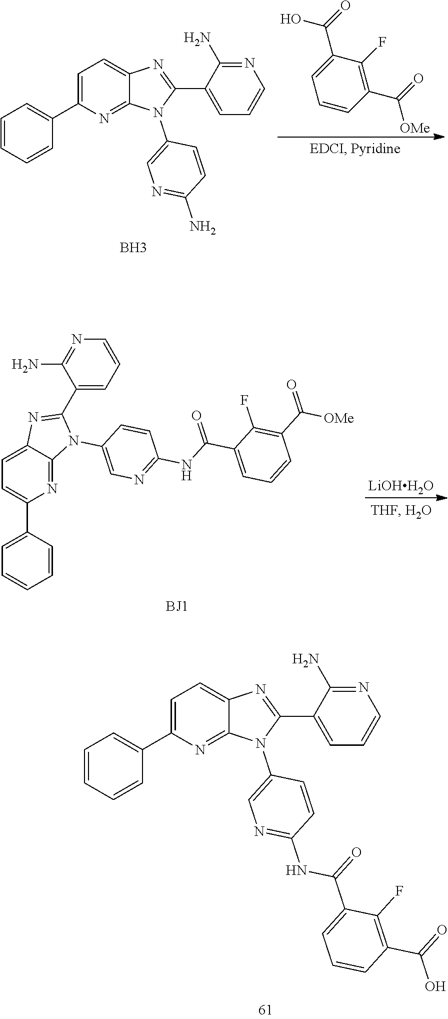

Step 1. To a mixture of 3-(3-(6-aminopyridin-3-yl)-5-phenyl-3H-imidazo[4,5-b]pyridin-2-yl)pyridin-2-amine BH3 (100 mg, 0.260 mmol) and 2-fluoro-3-methoxy carbonyl-benzoic acid (63.0 mg, 0.310 mmol) in pyridine (3.00 mL), EDCI (152 mg, 0.790 mmol) was added and the mixture was stirred at 80° C. for 2 hrs. Water (10 mL) was added to the mixture, and the resulting mixture was extracted with EtOAc (15 mL×3), and washed with brine (20 mL×2). The combined organic phase was dried over anhydrous sodium sulfate, filtered and the filtrate was concentrated. The crude product was purified by flash chromatography on silica gel (70% of EtOAc in PE) to afford methyl 3-((5-(2-(2-aminopyridin-3-yl)-5-phenyl-3H-imidazo[4,5-b]pyridin-3-yl)pyridin-2-yl)carbamoyl)-2-fluorobenzoate BJ1 (60.0 mg, 40.6% yield) as a yellow oil. LCMS Rₜ=0.828 min in 1.5 min chromatography, purity 20.7%, MS ESI calcd, for 559.18 [M+H]⁺ 560.18, found 560.1.

Step 2. To a solution of methyl 3-((5-(2-(2-aminopyridin-3-yl)-5-phenyl-3H-imidazo[4,5-b]pyridin-3-yl)pyridin-2-yl)carbamoyl)-2-fluorobenzoate BJ1 (60.0 mg, 0.110 mmol) in THF (2.00 mL) and water (2.00 mL), LiOH·H₂O (15.0 mg, 0.340 mmol) was added at 25° C. and the mixture was stirred at 25° C. for 2 hrs. HCl (2 M, 1 mL) was added and the mixture was purified by prep-HPLC (condition: water (FA)-ACN; Column: Xtimate C18 150*40 mm*10 μm; B %: 10%-50%; Gradient Time, 25 min) to afford 3-((5-(2-(2-aminopyridin-3-yl)-5-phenyl-3H-imidazo[4,5-b]pyridin-3-yl)pyridin-2-yl)carbamoyl)-2-fluorobenzoic acid 61 (2.60 mg, 3.82% yield) as a white solid. ¹H NMR (400 MHz, DMSO-d₆) δ=11.29 (s, 1H), 8.52 (s. 1H), 8.37 (d, J=8.4 Hz, 1H), 8.33-8.28 (m, 1H), 8.10-8.00 (m, 6H), 7.89 (t, J=4.8 Hz. 1H), 7.49 (t, J=6.8 Hz, 2H), 7.44-7.38 (m, 3H), 6.83 (s, 2H), 6.58-6.52 (m, 1H). HPLC Rₜ=2.736 min in 8 min chromatography, purity 93.1%. LCMS Rₜ=1.795 min in 4 min chromatography, purity 92.1%, MS ESI calcd, for 545.16 [M+H]⁺ 546.16, found 546.2.

### Example 62. Synthesis of 4-((5-(2-(2-aminopyridin-3-yl)-5-phenyl-3H-imidazo[4,5-b]pyridin-3-yl)pyridin-2-yl)carbamoyl)-3-fluorobenzoic acid (Compound 62)

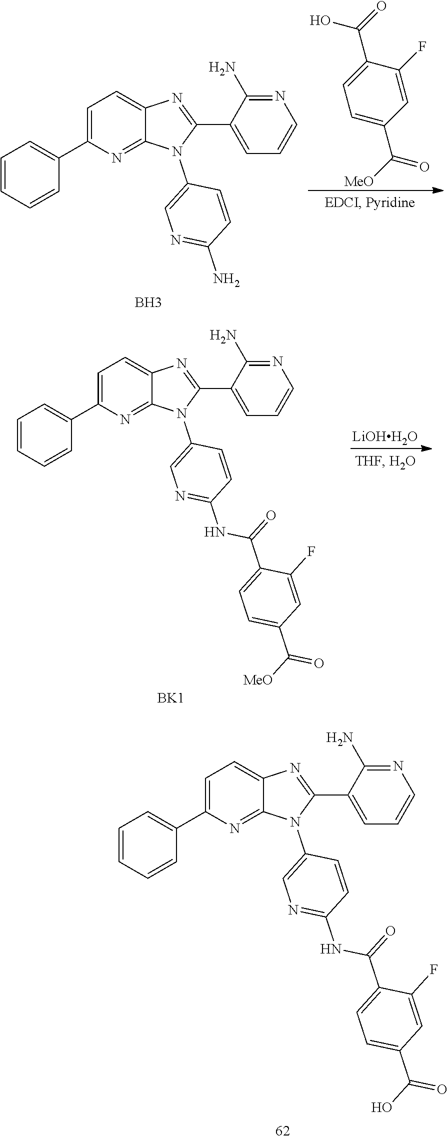

Step 1. To a mixture of 3-(3-(6-aminopyridin-3-yl)-5-phenyl-3H-imidazo[4,5-b]pyridin-2-yl)pyridin-2-amine BH3 (100 mg, 0.260 mmol) and 2-fluoro-4-(methoxycarbonyl)benzoic acid (63.0 mg, 0.310 mmol) in pyridine (3.00 mL), EDCI (152 mg, 0.790 mmol) was added and the mixture was stirred at 80° C. for 2 hrs. Water (10 mL) was added into the mixture and the resulting mixture was extracted with EtOAc (15 mL×3) and washed with brine (20 mL×2). The combined organic phase was dried over anhydrous sodium sulfate, filtered and the filtrate was concentrated. The crude product was purified by flash chromatography on silica gel (70% of EtOAc in PE) to afford methyl 4-((5-(2-(2-aminopyridin-3-yl)-5-phenyl-3H-imidazo[4,5-b]pyridin-3-yl)pyridin-2-yl)carbamoyl)-3-fluorobenzoate BK1 (70.0 mg, 47.4% yield) as a yellow oil. LCMS Rₜ=0.852 min in 1.5 min chromatography, purity 52.5%, MS ESI calcd, for 559.18 [M+H]⁺ 560.18, found 560.3.

Step 2. To a solution of methyl 4-((5-(2-(2-aminopyridin-3-yl)-5-phenyl-3H-imidazo[4,5-b]pyridin-3-yl)pyridin-2-yl)carbamoyl)-3-fluorobenzoate BK1 (70.0 mg, 0.120 mmol) in THF (2.00 mL) and water (2.00 mL), LiOH·H₂O (16.0 mg, 0.370 mmol) was added at 25° C. and the mixture was stirred for 2 hrs. HCl (2 M, 1 mL) was added, and the mixture was purified by prep-HPLC (condition: water (FA)-ACN; Column: Xtimate C18 150*40 mm*10 μm; B %: 12%-52%, Gradient Time, 25 min) to afford 4-((5-(2-(2-aminopyridin-3-yl)-5-phenyl-3H-imidazo[4,5-b]pyridin-3-yl)pyridin-2-yl)carbamoyl)-3-fluorobenzoic acid 62 (12.8 mg, 18.6% yield) as a white solid. ¹H NMR (400 MHz, DMSO-d₆) δ=13.50 (s, 1H), 11.31 (s, 1H), 8.52 (d, J=2.4 Hz, 1H), 8.36 (d, J=8.4 Hz, 1H), 8.31 (d, J=8.4 Hz, 1H), 8.12-8.01 (m, 5H), 7.89-7.81 (m, 2H), 7.79 (d, J=10.4 Hz, 1H), 7.49 (t, J=7.6 Hz, 2H), 7.45-7.39 (m, 2H), 6.83 (s, 2H), 6.55 (dd, J=4.8, 7.2 Hz, 1H). HPLC Rₜ=2.759 min in 8 min chromatography, purity 99.7%. LCMS Rₜ=1.833 min in 4 min chromatography, purity 99.8%, MS ESI calcd, for 545.16 [M+H]⁺ 546.16, found 546.2.

### Example 63. Synthesis of 4-[[5-[2-(2-amino-3-pyridyl)-5-phenyl-imidazo[4,5-b]pyridin-3-yl]-2-pyridyl]carbamoyl]-3-methyl-benzoic acid (Compound 63)

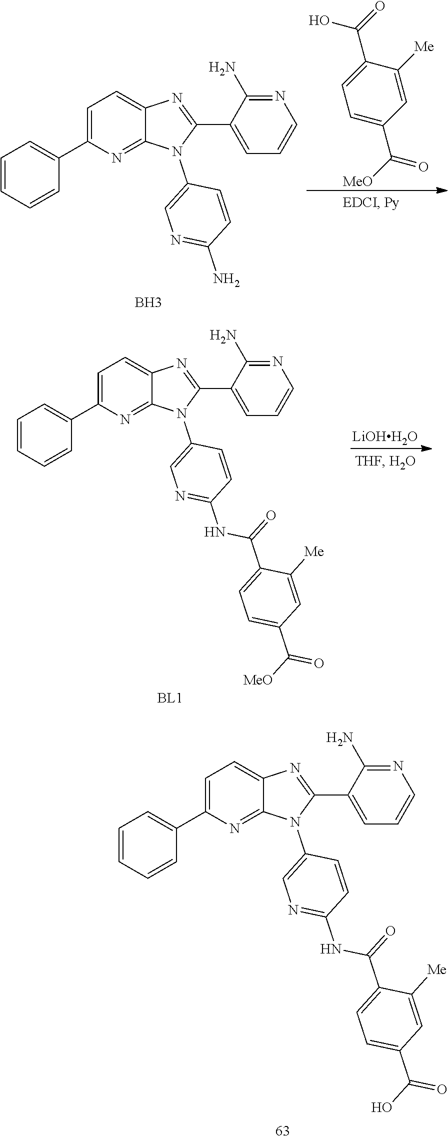

Step 1. To a mixture of 3-(3-(6-aminopyridin-3-yl)-5-phenyl-3H-imidazo[4,5-b]pyridin-2-yl)pyridin-2-amine BH3 (100 mg, 0.260 mmol) and 4-(methoxycarbonyl)-2-methylbenzoic acid (51.0 mg, 0.260 mmol) in pyridine (3.00 mL), EDCI (152 mg, 0.790 mmol) was added and the mixture was stirred at 80° C. for 2 hrs. Water (10 mL) was added and the resulting mixture was extracted with EtOAc (15 mL×3) and washed with brine (20 mL×2). The combined organic phase was dried over anhydrous sodium sulfate, filtered and the filtrate was concentrated and purified by flash chromatography on silica gel (72% of EtOAc in PE) to afford methyl 4-[[5-[2-(2-amino-3-pyridyl)-5-phenyl-imidazo[4,5-b]pyridin-3-yl]-2-pyridyl]carbamoyl]-3-methyl-benzoate BL1 (80.0 mg, 54.6% yield) as a yellow oil. LCMS Rₜ=0.803 min in 1.5 min chromatography, purity 82.6%, MS ESI calcd, for 555.20 [M+H]⁺ 556.20, found 556.0.

Step 2. To a solution of methyl 4-[[5-[2-(2-amino-3-pyridyl)-5-phenyl-imidazo[4,5-b]pyridin-3-yl]-2-pyridyl]carbamoyl]-3-methyl-benzoate BL1 (70.0 mg, 0.120 mmol) in THE (2.00 mL) and water (2.00 mL), LiOH·H₂O (16.0 mg, 0.370 mmol) was added at 25° C. and the mixture stirred for 2 hrs. Formic acid (1 mL) was added and the mixture was purified by prep-HPLC (condition: water (FA)-ACN; Column: Xtimate C18 150*40 mm*10 μm; B %: 10%-50%; Gradient Time, 25 min) to afford 4-[[5-[2-(2-amino-3-pyridyl)-5-phenyl-imidazo[4,5-b]pyridin-3-yl]-2-pyridyl]carbamoyl]-3-methyl-benzoic acid 63 (12.5 mg, 18.1% yield) as a white solid. ¹H NMR (400 MHz, DMSO-d₆) δ=11.16 (s, 1H), 8.49 (d, J=2.4 Hz, 1H), 8.37 (d, J=9.2 Hz, 1H), 8.30 (d, J=8.4 Hz, 1H), 8.08 (s, 1H), 8.07-8.00 (m, 4H), 7.88-7.80 (m, 2H), 7.60 (d, J=8.0 Hz, 1H), 7.52-7.46 (m, 2H), 7.44-7.38 (m, 2H), 6.79 (s, 2H), 6.55 (dd, J=4.8, 7.6 Hz, 1H), 2.46 (s, 3H). HPLC Rₜ=2.752 min in 8 min chromatography, purity 99.0%. LCMS Rₜ=1.826 min in 4 min chromatography, purity 99.5%, MS ESI calcd. for 541.19 [M+H]⁺ 542.19, found 542.2.

### Example 64. Synthesis of 2-(3-((5-(2-(2-aminopyridin-3-yl)-5-phenyl-3H-imidazo[4,5-b]pyridin-3-yl)pyridin-2-yl)carbamoyl)phenyl) acetic acid (Compound 64)

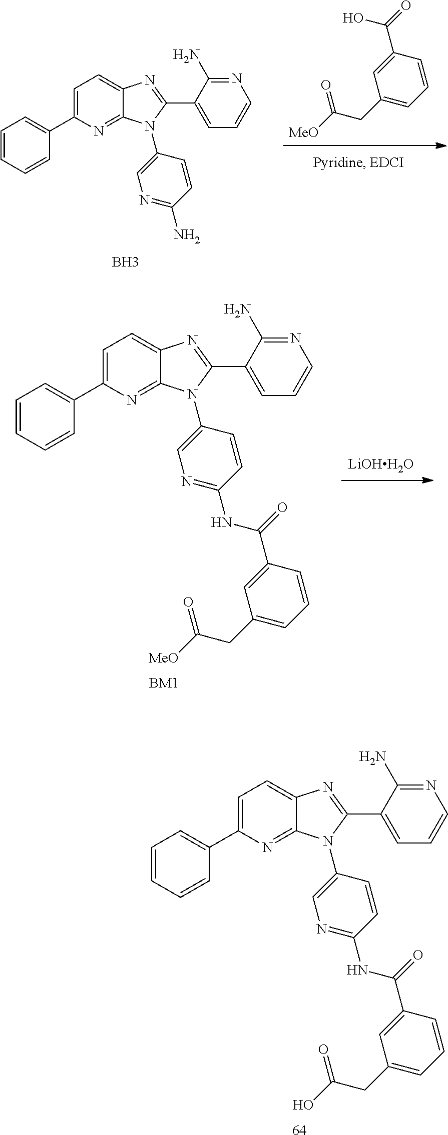

Step 1. To a mixture of 3-(3-(6-aminopyridin-3-yl)-5-phenyl-3H-imidazo[4,5-b]pyridin-2-yl)pyridin-2-amine BH3 (100 mg, 0.260 mmol) and 3-(2-methoxy-2-oxoethyl)benzoic acid (77.0 mg, 0.390 mmol) in pyridine (2.00 mL), EDCI (152 mg, 0.790 mmol) was added and the mixture was stirred at 80° C. for 2 hrs. Water (10 mL) was added, and the resulting mixture was extracted with EtOAc (15 mL×3). The combined organic phase was dried over anhydrous Na₂SO₄, filtered and concentrated. The residue was purified by flash column (EtOAc in PE=0%-80%) to afford methyl 2-(3-((5-(2-(2-aminopyridin-3-yl)-5-phenyl-3H-imidazo[4,5-b]pyridin-3-yl)pyridin-2-yl)carbamoyl)phenyl)acetate BM1 (40.0 mg, 27.3% yield) as a yellow solid. LCMS Rₜ=1.378 min in 2 min chromatography, purity 51.3%, MS ESI calcd, for 555.20, [M+H]⁺ 556.20, found 556.0.

Step 2. To a solution of methyl 2-(3-((5-(2-(2-aminopyridin-3-yl)-5-phenyl-3H-imidazo[4,5-b]pyridin-3-yl)pyridin-2-yl)carbamoyl)phenyl)acetate BM1 (40.0 mg, 0.0700 mmol) in THF (1.00 mL) and water (1.00 mL), LiOH·H₂O (4.50 mg, 0.100 mmol) was added at 25° C. and the mixture was stirred for 2 hrs. HCl (2 M, 1 mL) was added and the mixture was purified by prep-HPLC (condition: water (FA)-ACN; Column: Xtimate C18 150*40 mm*10 μm; B %: 8%-48%; Gradient Time, 25 min) to afford 2-(3-((5-(2-(2-aminopyridin-3-yl)-5-phenyl-3H-imidazo[4,5-b]pyridin-3-yl)pyridin-2-yl)carbamoyl)phenyl) acetic acid 64 (7.20 mg, 17.8% yield) as a white solid. ¹H NMR (DMSO-d₆, 400 MHz) δ_{H}=12.42 (s, 1H), 11.05 (s, 1H), 8.53 (d, J=2.4 Hz, 1H), 8.37 (d, J=9.2 Hz, 1H), 8.31 (d, J=8.4 Hz, 1H), 8.11-8.01 (m, 5H), 7.99-7.93 (m, 2H), 7.55-7.38 (m, 6H), 6.91 (s, 2H), 6.59-6.52 (m, 1H), 3.70 (s, 2H). HPLC Rₜ=2.740 min in 8 min chromatography, purity 96.5%. LCMS R$=1.715 min in 4 min chromatography, purity 95.4%, MS ESI calcd, for 541.19, [M+H]⁺ 542.19, found 542.2.

### Example 65. Synthesis of 3-((4-(2-(2-aminopyridin-3-yl)-5-phenyl-3H-imidazo[4,5-b]pyridin-3-yl)-2-fluorophenyl)carbamoyl)-2-fluorobenzoic acid (Compound 65)

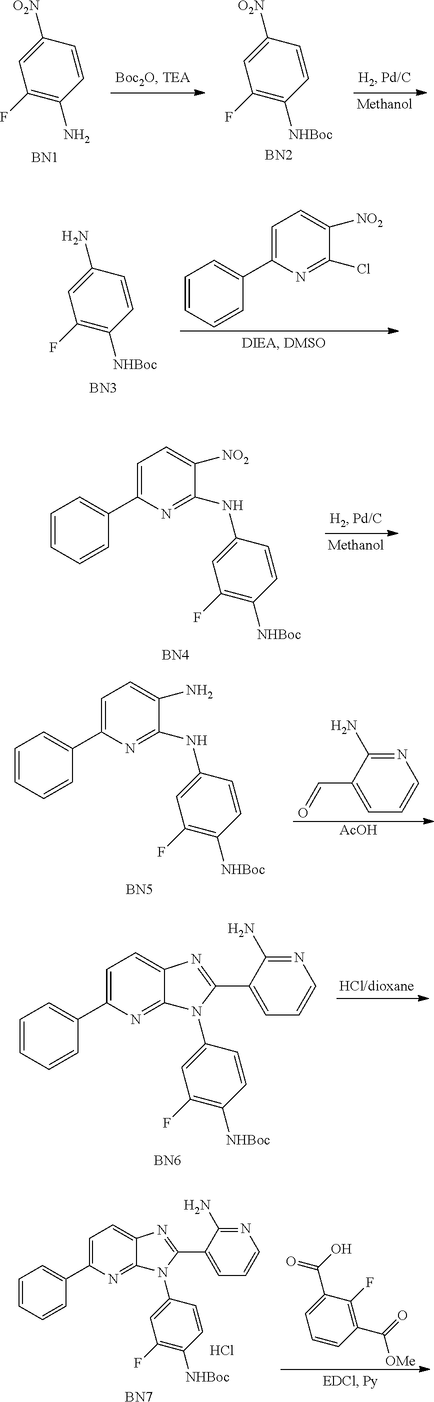

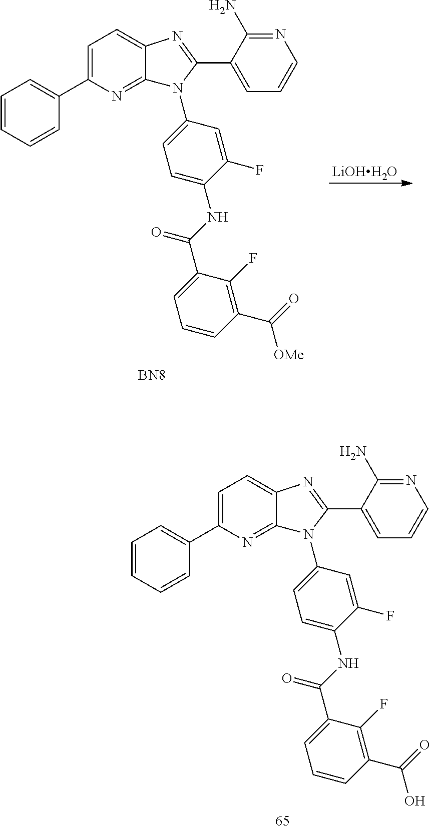 [SMILES: COC(=O)c1cccc(C(=O)Nc2ccc(-n3c(-c4cccnc4N)nc4ccc(-c5ccccc5)nc43)cc2F)c1F ; "LiOH·H2O" ; "BN8" ; Nc1ncccc1-c1nc2ccc(-c3ccccc3)nc2n1-c1ccc(NC(=O)c2cccc(C(=O)O)c2F)c(F)c1 ; "65"]

Step 1. To a solution of 2-fluoro-4-nitro-aniline BN1 (5.00 g, 32.0 mmol) in DCM (50.0 mL), TEA (9.72 g, 96.1 mmol) and Boc₂O (10.5 g, 48.0 mmol) were added at 25° C., and the mixture was stirred at 50° C. for 2 hrs. The mixture was diluted with DCM (100 mL) and washed with water (30 mL×2). The organic layer was dried over Na₂SO₄, filtered, and concentrated. The crude product was purified by column chromatography on silica gel (EtOAc in PE, 0-5%) to afford tert-butyl N-(2-fluoro-4-nitro-phenyl)carbamate BN2 (5.60 g, 68.2% yield) as a yellow oil. ¹H NMR (DMSO-d₆, 400 MHz) δ_{H}=9.73 (s, 1H), 8.15-8.03 (m, 3H), 1.48 (s, 9H).

Step 2. To a solution of tert-butyl N-(2-fluoro-4-nitro-phenyl)carbamate BN2 (5.60 g, 21.9 mmol) in methanol (20.0 mL), wet Pd/C (2.00 g, 10% purity) was added under an Ar atmosphere. The suspension was degassed and purged with H₂ (×3), and the reaction mixture was stirred under H₂ (15 psi) at 25° C. for 10 hrs. The reaction mixture was filtered via a celite pad, and the filter cake was washed with DCM/MeOH (10/1 (500 mL)). The filtrate was concentrated to afford tert-butyl N-(4-amino-2-fluoro-phenyl)carbamate BN3 (3.80 g, 76.9% yield) as a yellow oil, which was used in the next step directly. ¹H NMR (DMSO-d₆, 400 MHz) δ_{H}=8.28 (s, 1H), 6.95 (s, 1H), 6.37-6.25 (m, 2H), 5.22 (s, 2H), 1.48-1.32 (s, 9H).

Step 3. To a solution of tert-butyl (4-amino-2-fluorophenyl)carbamate BN3 (2.30 g, 9.80 mmol) in DMSO (20.0 mL), 2-chloro-3-nitro-6-phenylpyridine (2.66 g. 11.8 mmol) and DIEA (3.80 g, 29.4 mmol) were added at 25° C., and the mixture was stirred at 105° C. for 14 hrs. The reaction mixture was diluted with EtOAc (30 mL), washed with brine (10 mL×3), dried over anhydrous Na₂SO₄, filtered and concentrated. The residue was purified by flash silica gel chromatography (ISCOR; 40 g, SepaFlash® Silica Flash Column, eluent of 0-10% EtOAc in PE) to afford tert-butyl(2-fluoro-4-((3-nitro-6-phenylpyridin-2-yl)amino)phenyl)carbamate BN4 (1.50 g, 36.1% yield) as a red solid. ¹H NMR (DMSO-d₆, 400 MHz) δ_{H}=10.11 (s, 1H), 8.93 (s, 1H), 8.62 (d, J=8.8 Hz, 1H), 8.09 (dd, J=2.8, 6.4 Hz, 2H), 7.76 (dd, J=2.0, 13.2 Hz, 1H), 7.62 (d, J=8.8 Hz, 1H), 7.59-7.52 (m, 4H), 7.50-7.45 (m, 1H), 1.47 (s, 9H).

Step 4. To a solution of tert-butyl(2-fluoro-4-((3-nitro-6-phenylpyridin-2-yl)amino)phenyl)carbamate BN4 (480 mg, 1.13 mmol) in methanol (4.00 mL) and THF (4.00 mL), wet Pd/C (120 mg, 10%) was added under an Ar atmosphere. The suspension was degassed and purged with H₂ (×3), and the reaction mixture was stirred under H₂ (15 Psi) at 25° C. for 12 hrs. The reaction mixture was filtered and the filter cake was washed with MeOH/DCM (1/1, 500 mL). The resulting suspension was concentrated directly to afford tert-butyl (4-((3-amino-6-phenylpyridin-2-yl)amino)-2-fluorophenyl)carbamate BN5 (470 mg, crude) as a yellow solid. LCMS Rₜ=1.480 min in 2.5 min chromatography, purity 55.6%, MS ESI calcd, for 394.18 [M+H]⁺ 395.18, found 395.0.

Step 5. A solution of 2-aminopyridine-3-carbaldehyde (161 mg, 1.32 mmol) and tert-butyl (4-((3-amino-6-phenylpyridin-2-yl)amino)-2-fluorophenyl)carbamate BN5 (400 mg, 1.01 mmol) in acetic acid (3.0 mL) was stirred at 100° C. for 1 hr. The mixture was concentrated directly and the resulting residue was purified by flash silica gel chromatography (ISCOR; 20 g, SepaFlash® Silica Flash Column, eluent with 0-50% of EtOAc in PE) to afford tert-butyl (4-(2-(2-aminopyridin-3-yl)-5-phenyl-3H-imidazo[4,5-b]pyridin-3-yl)-2-fluorophenyl)carbamate BN6 (115 mg, 18.7% yield) as a yellow solid. ¹H NMR (DMSO-d₆, 400 MHz) δ=9.24 (s, 1H), 8.27 (d, J=8.4 Hz, 1H), 8.06-7.97 (m, 4H), 7.80 (t, J=8.4 Hz, 1H), 7.53-7.45 (m, 3H), 7.43-7.37 (m, 1H), 7.30 (dd, J=1.6, 7.6 Hz, 1H), 7.25 (dd, J=1.6, 8.8 Hz, 1H), 6.91 (s, 2H), 6.48 (dd, J=4.8, 7.6 Hz, 1H), 1.48 (s, 9H).

Step 6. To a solution of tert-butyl (4-(2-(2-aminopyridin-3-yl)-5-phenyl-3H-imidazo[4,5-b]pyridin-3-yl)-2-fluorophenyl)carbamate BN6 (115 mg, 0.231 mmol) in DCM (1.00 mL), HCl/dioxane (1.00 mL, 4 M) was added and the mixture was stirred at 20° C. for 1 hr. The mixture was concentrated directly to afford 3-(3-(4-amino-3-fluorophenyl)-5-phenyl-3H-imidazo[4,5-b]pyridin-2-yl)pyridin-2-amine hydrochloride BN7 (100 mg, crude) as a yellow solid. ¹H NMR (DMSO-d₆, 400 MHz) δ=8.74-8.40 (m, 2H), 8.33 (dd, J=2.8, 8.4 Hz. 1H), 8.13 (d, J=5.6 Hz, 1H), 8.05 (d, J=3.2 Hz, 4H), 7.92 (d, J=6.8 Hz, 1H), 7.53-7.41 (m, 4H), 7.35 (d, J=11.6 Hz, 1H), 7.07 (d, J=8.0 Hz, 1H), 6.97-6.89 (m, 2H).

Step 7. To a solution of 3-(3-(4-amino-3-fluorophenyl)-5-phenyl-3H-imidazo[4,5-b]pyridin-2-yl)pyridin-2-amine hydrochloride BN7 (150 mg, 0.380 mmol) and 2-fluoro-3-(methoxycarbonyl)benzoic acid (75.0 mg, 0.380 mmol) in pyridine (2.00 mL), EDCI (218 mg, 1.14 mmol) was added and the mixture was stirred at 80° C. for 2 hrs. The mixture was diluted with EtOAc (50 mL), washed with brine (20 mL×3). The combined organic phase was dried over anhydrous Na₂SO₄, filtered and concentrated. The residue was purified by flash silica gel chromatography (ISCOR; 4 g, SepaFlash® Silica Flash Column, eluent with 0-70% of EtOAc in PE) to afford methyl 3-((4-(2-(2-aminopyridin-3-yl)-5-phenyl-3H-imidazo[4,5-b]pyridin-3-yl)-2-fluorophenyl)carbamoyl)-2-fluorobenzoate BN8 (62.0 mg, 28.4% yield) as a yellow solid. ¹H NMR (DMSO-d₆, 400 MHz) δ=10.57 (s, 1H), 8.29 (d, J=8.4 Hz, 1H), 8.10-7.99 (m, 6H), 7.96 (t, J=6.8 Hz, 1H), 7.66-7.61 (m, 1H), 7.48 (q, J=8.0 Hz, 3H), 7.44-7.33 (m, 3H), 6.89 (s, 2H), 6.52 (dd, J=4.8, 7.6 Hz, 1H), 3.90 (s, 3H).

Step 8. A solution of methyl 3-((4-(2-(2-aminopyridin-3-yl)-5-phenyl-3H-imidazo[4,5-b]pyridin-3-yl)-2-fluorophenyl)carbamoyl)-2-fluorobenzoate BN8 (63.0 mg, 0.110 mmol) and LiOH·H₂O (14.0 mg, 0.330 mmol) in water (1.00 mL) and THE (1.00 mL) was stirred at 25° C. for 2 hrs. The mixture was filtered directly and the mixture was purified by prep-HPLC (column: Xtimate C18 150*40 mm*10 μm; mobile phase: [water (NH₄HCO₃)-ACN]; B %: 4%-44%, 25 min) to afford 3-((4-(2-(2-aminopyridin-3-yl)-5-phenyl-3H-imidazo[4,5-b]pyridin-3-yl)-2-fluorophenyl)carbamoyl)-2-fluorobenzoic acid 65 (22.0 mg, 35.6% yield) as a yellow solid. ¹H NMR (DMSO-d₆, 400 MHz) δ=10.50 (s, 1H), 8.29 (d, J=8.4 Hz, 1H), 8.09-7.94 (m, 6H), 7.91-7.82 (m, 1H), 7.63 (dd, J=2.0, 11.2 Hz, 1H), 7.52-7.46 (m, 2H), 7.44-7.32 (m, 4H), 6.89 (s, 2H), 6.52 (dd, J=4.8, 7.6 Hz, 1H). HPLC Rₜ=3.190 min in 8 min chromatography, purity 99.4%. LCMS Rₜ=1.867 min in 4 min chromatography, purity 97.9%, MS ESI calcd, for 562.16 [M+H]⁺ 563.16, found 563.2.

### Example 66. Synthesis of 4-((4-(2-(2-aminopyridin-3-yl)-5-phenyl-3H-imidazo[4,5-b]pyridin-3-yl)-2-fluorophenyl)carbamoyl)-3-methylbenzoic acid (Compound 66)

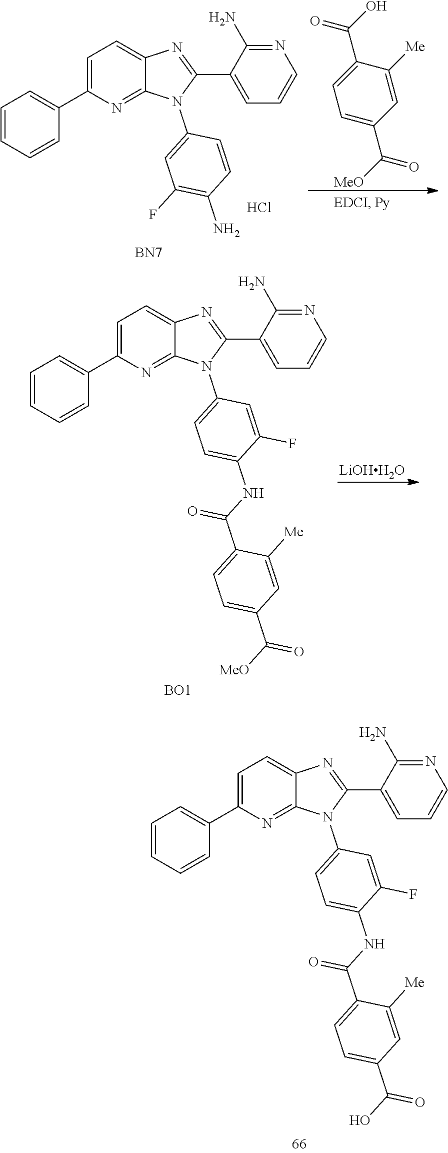

Step 1. To a solution of 3-(3-(4-amino-3-fluorophenyl)-5-phenyl-3H-imidazo[4,5-b]pyridin-2-yl)pyridin-2-amine hydrochloride BN7 (150 mg, 0.380 mmol) and 4-(methoxy carbonyl)-2-methylbenzoic acid (73.0 mg, 0.380 mmol) in pyridine (2.00 mL), EDCI (218 mg, 1.14 mmol) was added and the mixture was stirred at 80° C. for 2 hrs. The mixture was diluted with EtOAc (5 mL) and washed with brine (2 mL×3). The combined organic phase was dried over anhydrous Na₂SO₄, filtered and concentrated. The residue was purified by flash silica gel chromatography (ISCO®; 4 g, SepaFlash® Silica Flash Column, eluent with 0-60% of EtOAc in PE) to afford methyl 4-((4-(2-(2-aminopyridin-3-yl)-5-phenyl-3H-imidazo[4,5-b]pyridin-3-yl)-2-fluorophenyl)carbamoyl)-3-methylbenzoate BO1 (87.0 mg, 40.1% yield) as a yellow solid. ¹H NMR (DMSO-d₆, 400 MHz) δ=10.46 (s, 1H), 8.29 (d, J=8.4 Hz, 1H), 8.09-7.99 (m, 4H), 7.96-7.86 (m, 3H), 7.68-7.59 (m, 2H), 7.52-7.46 (m, 2H), 7.45-7.33 (m, 3H), 6.85 (s, 2H), 6.52 (dd, J=4.8, 7.6 Hz, 1H), 3.89 (s, 3H), 2.56 (s, 3H).

Step 2. A solution of methyl 4-((4-(2-(2-aminopyridin-3-yl)-5-phenyl-3H-imidazo[4,5-b]pyridin-3-yl)-2-fluorophenyl)carbamoyl)-3-methylbenzoate BO1 (82.0 mg, 0.140 mmol) and LiOH H₂O (18.0 mg, 0.430 mmol) in water (1.0 mL) and THF (1.0 mL) was stirred at 25° C. for 2 hrs. The reaction mixture was acidified to pH ~4 with aqueous HCl (2 M). The mixture was diluted with ACN (3 mL) and purified by prep-HPLC (column: Xtimate C18 150*40 mm*10 μm; mobile phase: [water (HCl)-ACN]; B %: 10%-50%, 36 min) to afford 4-((4-(2-(2-aminopyridin-3-yl)-5-phenyl-3H-imidazo[4,5-b]pyridin-3-yl)-2-fluorophenyl)carbamoyl)-3-methylbenzoic acid 66 (35.7 mg, 42.3% yield) as a yellow solid. ¹H NMR (MeOD, 400 MHz) δ=8.32 (d, J=8.4 Hz, 1H), 8.19 (t, J=8.4 Hz, 1H), 8.09-8.01 (m, 4H), 8.00-7.93 (m, 3H), 7.67-7.56 (m, 2H), 7.50-7.39 (m, 4H), 6.94 (t, J=6.8 Hz, 1H), 2.56 (s, 3H). HPLC Rₜ=3.183 min in 8 min chromatography, purity 94.9%. LCMS Rₜ=1.873 min in 4 min chromatography, purity 92.5%, MS ESI calcd, for 558.18 [M+H]⁺ 559.18, found 559.2.

### Example 67. Synthesis of 2-(3-((4-(2-(2-aminopyridin-3-yl)-5-phenyl-3H-imidazo[4,5-b]pyridin-3-yl)-2-fluorophenyl)carbamoyl)phenyl) acetic acid (Compound 67)

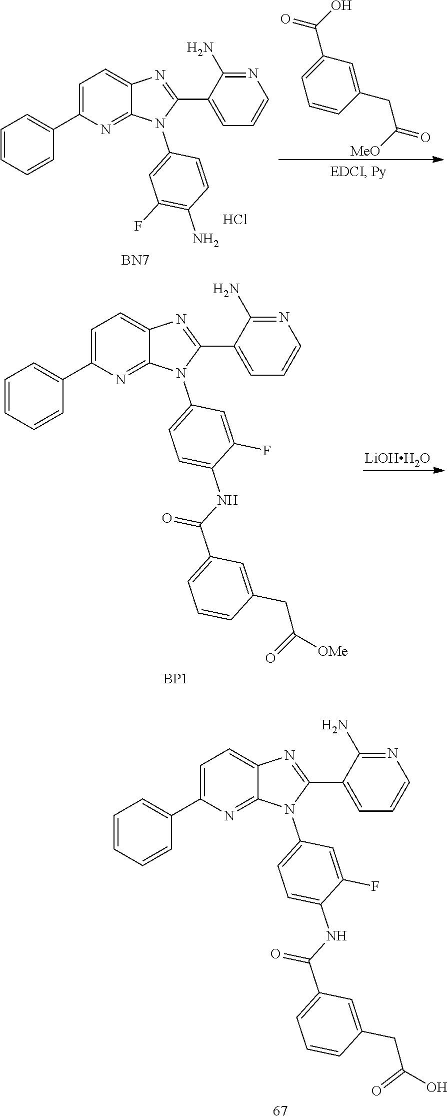

Step 1. To a solution of 3-(2-methoxy-2-oxo-ethyl)benzoic acid (49.0 mg, 0.252 mmol) and 3-(3-(4-amino-3-fluorophenyl)-5-phenyl-3H-imidazo[4,5-b]pyridin-2-yl)pyridin-2-amine hydrochloride BN7 (100 mg, 0.252 mmol) in pyridine (1.00 mL), EDCI (145 mg, 0.757 mmol) was added and the mixture was stirred at 80° C. for 2 hrs. Water (2 mL) was added to the reaction mixture and the resulting mixture was extracted with EtOAc (6 mL×3). The combined organic phase was washed with brine (4 mL×2), dried over anhydrous Na₂SO₄, filtered and concentrated. The residue was purified by flash silica gel chromatography (ISCO®; 4 g, SepaFlash® Silica Flash Column, eluent with 0-70% of EtOAc in PE) to afford methyl 2-(3-((4-(2-(2-aminopyridin-3-yl)-5-phenyl-3H-imidazo[4,5-b]pyridin-3-yl)-2-fluorophenyl)carbamoyl)phenyl)acetate BP1 (65.0 mg, 36.2% yield) as a yellow solid. ¹H NMR (DMSO-d₆, 400 MHz) δ=10.30 (s, 1H), 8.29 (d, J=8.4 Hz, 1H), 8.08-8.01 (m, 4H), 7.93-7.88 (m, 2H), 7.79 (t, J=8.4 Hz, 1H), 7.63 (dd, J=2.0, 11.2 Hz, 1H), 7.57-7.45 (m. 5H), 7.44-7.39 (m, 1H), 7.35 (dd, J=1.6, 7.6 Hz, 2H), 6.89 (s, 2H), 3.81 (s, 2H), 3.64 (s, 3H).

Step 2. A solution of methyl 2-[3-[[4-[2-(2-amino-3-pyridyl)-5-phenyl-imidazo[4,5-b]pyridin-3-yl]-2-fluoro-phenyl]carbamoyl]phenyl]acetate BP1 (65.0 mg, 0.120 mmol) and LiOH·H₂O (9.50 mg, 0.227 mmol) in THF (1.00 mL) and water (1.00 mL) was stirred at 25° C. for 2 hrs. The reaction mixture was acidified to pH ~4 with aqueous HCl (2 M). The resulting mixture was dissolved in ACN (3 mL) and purified by prep-HPLC (column: Xtimate C18 150*40 mm*10 μm; mobile phase: [water (HCl)-ACN]; B %: 10%-50%, 36 min) to afford 2-(3-((4-(2-(2-aminopyridin-3-yl)-5-phenyl-3H-imidazo[4,5-b]pyridin-3-yl)-2-fluorophenyl)carbamoyl)phenyl) acetic acid 67 (23.7 mg, 36.1% yield) as a yellow solid. ¹H NMR (DMSO-d₆, 400 MHz) δ=10.33 (s, 1H), 8.47-8.26 (m, 2H), 8.16 (dd, J=1.2, 6.0 Hz, 1H), 8.13-8.05 (m, 3H), 7.99 (d, J=6.4 Hz, 1H), 7.90 (s, 2H), 7.84 (1, J=8.4 Hz, 1H), 7.69 (dd, J=2.0, 11.0 Hz, 1H), 7.55-7.40 (m, 6H), 6.96 (t, J=6.8 Hz, 1H), 3.70 (s, 2H). HPLC Rₜ=3.204 min in 8 min chromatography, purity 96.5%. LCMS Rₜ=1.966 min in 4 min chromatography, purity 95.4%, MS ESI calcd, for 558.18 [M+H]⁺ 559.18, found 559.2.

### Example 68. Synthesis of 4-[[5-[2-(2-amino-3-pyridyl)-5-phenyl-imidazo[4,5-b]pyridin-3-yl]-2-pyridyl]amino]methyl]-3-fluoro-benzoic acid (Compound 68)

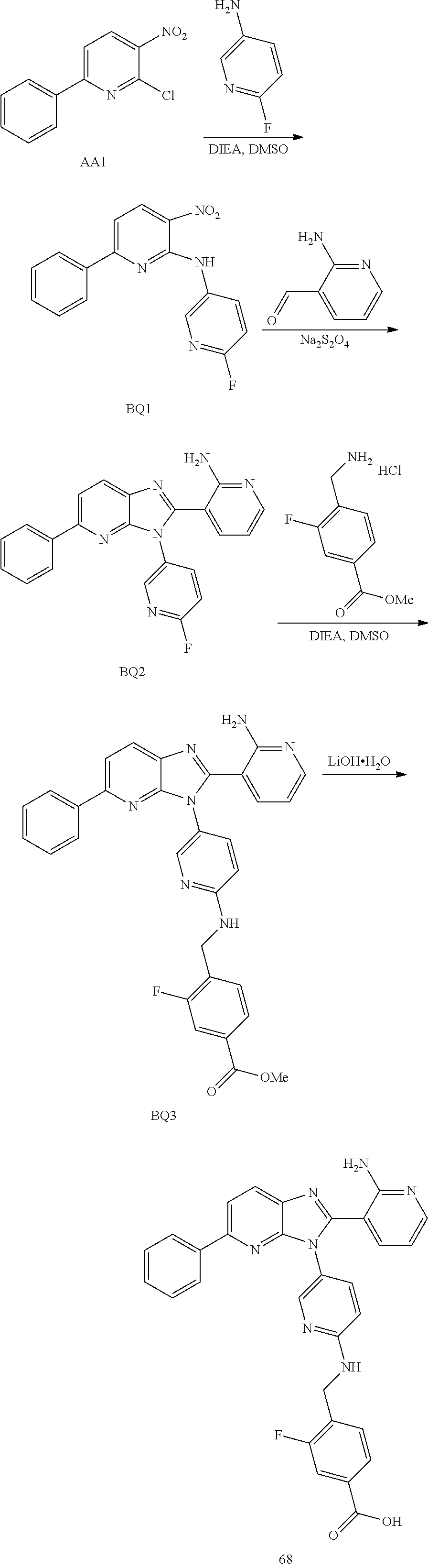

Step 1. To a stirred solution of 2-chloro-3-nitro-6-phenyl-pyridine AA1 (2.00 g, 8.52 mmol) and 6-fluoropyridin-3-amine (1.91 g, 17.0 mmol) in DMSO (30.0 mL), DIEA (4.70 mL, 25.6 mmol) was added, and the reaction was stirred at 90° C. under N₂ for 16 hrs. The reaction mixture was diluted with EtOAc (100 mL), washed with water (50 mL), brine (100 mL×2), dried over anhydrous sodium sulfate, filtered and the filtrate was concentrated to afford a residue. The crude product was purified by flash chromatography on silica gel (30-95% EtOAc in PE) to afford N-(6-fluoro-3-pyridyl)-3-nitro-6-phenyl-pyridin-2-amine BQ1 (800 mg, 30.3% yield) as a yellow solid. ¹H NMR (400 MHz, CDCl₃) δ=10.18 (s, 1H), 8.71-8.53 (m, 2H), 8.30-8.13 (m, 1H), 8.06-7.91 (m, 2H), 7.56-7.45 (m, 3H), 7.38 (d, J=8.8 Hz, 1H), 7.02 (dd, J=3.2, 8.8 Hz, 1H)

Step 2. To a stirred solution of 2-aminopyridine-3-carbaldehyde (0.530 g, 4.35 mmol) and N-(6-fluoro-3-pyridyl)-3-nitro-6-phenyl-pyridin-2-amine BQ1 (1.00 g. 3.22 mmol) in DMSO (20.0 mL) and methanol (1.00 mL), Na₂S₂O₄ (1.68 g. 9.67 mmol) was added, and the reaction mixture was stirred at 90° C. under N₂ for 16 hrs. The reaction mixture was diluted with EtOAc (50 mL), washed with brine (50 mL×2), dried over anhydrous sodium sulfate, filtered and the filtrate was concentrated to afford a residue. The crude product was purified by flash chromatography on silica gel (90% EtOAc in PE) to afford 3-[3-(6-fluoro-3-pyridyl)-5-phenyl-imidazo[4,5-b]pyridin-2-yl]pyridin-2-amine BQ2 (500 mg, 40.6% yield) as a yellow solid. ¹H NMR (400 MHz, CDCl₃) δ=8.44 (d, J=2.0 Hz, 1H), 8.21-8.08 (m, 2H), 8.06-7.97 (m, 2H), 7.91-7.80 (m, 2H), 7.54-7.36 (m, 3H), 7.12 (dt, J=2.4, 8.0 Hz, 2H), 6.54 (s, 2H), 6.48 (dd, J=4.8, 7.6 Hz, 1H).

Step 3. To a stirred solution of 3-[3-(6-fluoro-3-pyridyl)-5-phenyl-imidazo[4,5-b]pyridin-2-yl]pyridin-2-amine BQ2 (100 mg, 0.260 mmol) and methyl 4~ (aminomethyl)-3-fluoro-benzoate hydrochloride (144 mg, 0.790 mmol) in DMSO (2.00 mL), DIEA (169 mg, 1.31 mmol) was added and the reaction mixture was stirred at 100° C. under N₂ for 16 hrs. The reaction mixture was diluted with EtOAc (20 mL), washed with water (15 mL) and brine (15 mL×2), dried over anhydrous sodium sulfate, filtered, and the filtrate was concentrated to afford methyl 4-[[[5-[2-(2-amino-3-pyridyl)-5-phenyl-imidazo[4,5-b]pyridin-3-yl]-2-pyridyl]amino methyl]-3-fluoro-benzoate BQ3 (60.0 mg, 42.1% yield) as a yellow oil. LCMS Rₜ=0.52 min in 1.0 min chromatography, purity 38.0%, MS ESI calcd, for 545.20 [M+H]⁺ 546.20, found 546.2.

Step 4. To a stirred solution of methyl 4-[[[5-[2-(2-amino-3-pyridyl)-5-phenyl-imidazo[4,5-b]pyridin-3-yl]-2-pyridyl]amino]methyl]-3-fluoro-benzoate BQ3 (60.0 mg, 0.110 mmol) in THF (1.00 mL) and water (1.00 mL), LiOH·H₂O (8.80 mg, 0.220 mmol) was added and the reaction mixture was stirred at 25° C. under N₂ for 5 hrs. The reaction mixture was diluted with water (10 mL) and washed with EtOAc (10 mL). The water phase was collected and adjusted to pH ~5 with 1N HCL. The mixture was purified by prep-HPLC (column: welch xtimate C18 150×30 mm×5 μm, method: water (FA)-ACN, begin B: 27, end B: 57) to afford 4-[[[5-[2-(2-amino-3-pyridyl)-5-phenyl-imidazo[4,5-b]pyridin-3-yl]-2-pyridyl]amino]methyl]-3-fluoro-benzoic acid 68 (5.80 mg, 9.80% yield) as a white solid. ¹H NMR (400 MHz, DMSO-d₆) δ=13.07 (s, 1H), 8.24 (d, J=8.4 Hz, 1H), 8.09-7.92 (m, 5H), 7.76 (dd, J=1.6, 8.0 Hz, 1H), 7.65 (dd, J=1.2, 10.8 Hz, 1H), 7.59-7.32 (m, 7H), 6.92 (s, 2H), 6.72 (d, J=8.8 Hz, 1H), 6.49 (dd, J=4.8, 7.6 Hz, 1H), 4.63 (d, J=5.6 Hz, 2H), 19F NMR (400 MHz, DMSO-d₆) δ=−118.181. HPLC Rₜ=3.560 min in 8 min chromatography, purity 98.6%. LCMS Rₜ=2.001 min in 4 min chromatography, purity 98.9%, MS ESI calcd. for 531.18 [M+H]⁺ 532.18, found 532.3.

### Example 69. Synthesis of methyl 4-(3-(4-(2-(2-aminopyridin-3-yl)-5-phenyl-3H-imidazo[4,5-b]pyridin-3-yl)phenyl) azetidine-1-carbonyl)benzoate (Compound 69)

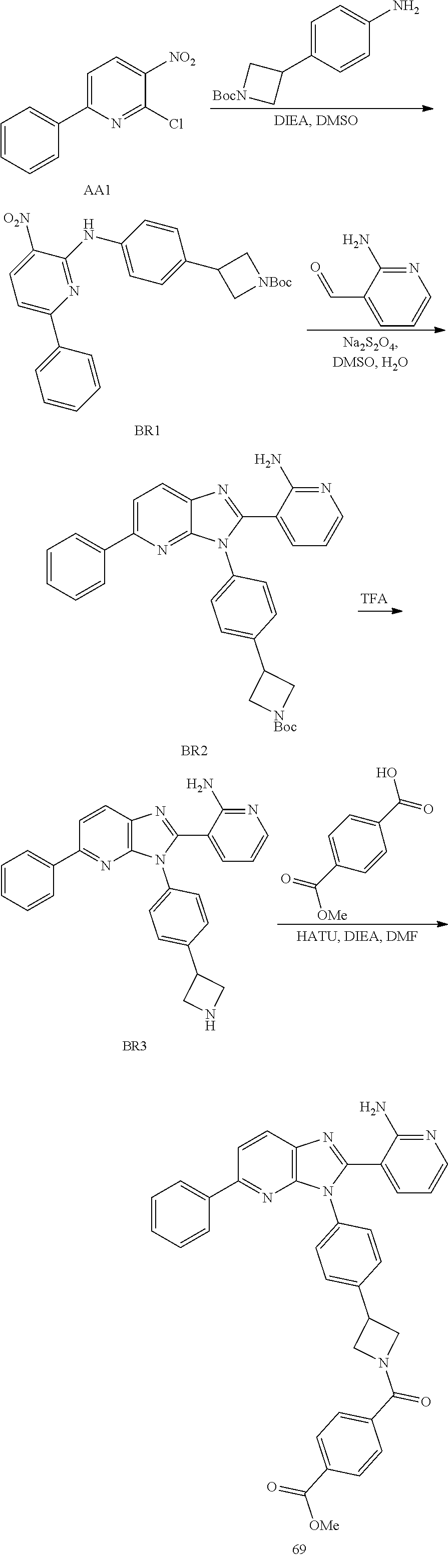

Step 1. To a solution of 2-chloro-3-nitro-6-phenyl-pyridine AA1 (1.80 g, 7.67 mmol) in DMSO (15.0 mL), tert-butyl 3-(4-aminophenyl) azetidine-1-carboxylate (2.00 g, 8.05 mmol) and DIEA (2.10 mL, 12.3 mmol) were added at 25° C., and the mixture was stirred at 80° C. for 16 hrs. Water (50 mL) was added and the resulting mixture was extracted with EtOAc (20 mL×3). The combined organic phase was washed with brine (50 mL), water (50 mL), dried over anhydrous Na₂SO₄, filtered and concentrated. The residue was purified by flash silica gel chromatography (eluent with 0-33% of EtOAc in PE) to give tert-butyl 3-(4-((3-nitro-6-phenylpyridin-2-yl)amino)phenyl) azetidine-1-carboxylate BR1 (1.20 g, 67.3% yield) as a red solid. ¹H NMR (400 MHz, CHLOROFORM-d) δ=10.31 (s, 1H), 8.59 (d, J=8.8 Hz, 1H), 8.10-8.00 (m, 2H), 7.76 (d, J=8.4 Hz, 2H), 7.57-7.45 (m, 3H), 7.38 (d, J=8.4 Hz, 2H), 7.31 (d, J=8.8 Hz, 1H), 4.36 (t, J=8.8 Hz, 2H), 4.02 (dd, J=8.4, 6.0 Hz, 2H), 3.82-3.73 (m, 1H), 1.49 (s, 9H).

Step 2. To a solution of tert-butyl 3-(4-(3-nitro-6-phenylpyridin-2-yl)amino)phenyl) azetidine-1-carboxylate BR1 (2.30 g, 5.15 mmol) in DMSO (23.0 mL) and methanol (23.0 mL), 2-aminonicotinaldehyde (849 mg, 6.95 mmol) and Na₂S₂O₄ (1.79 g, 10.3 mmol) were added at 25° C., and the mixture was stirred at 100° C. for 16 hrs. The reaction mixture was concentrated to remove MeOH. Water (50 mL) was added to the residue and the resulting mixture was extracted with EtOAc (100 mL×3). The combined organic phase was washed with brine (20 mL), water (20 mL), dried over anhydrous Na₂SO₄, filtered and concentrated. The residue was purified by flash silica gel chromatography (eluent with 0-66% of EtOAc in PE) to give tert-butyl 3-(4-(2-(2-aminopyridin-3-yl)-5-phenyl-3H-imidazo[4,5-b]pyridin-3-yl)phenyl) azetidine-1-carboxylate BR2 (400 mg, 17.1% yield) as a yellow oil. ¹H NMR (400 MHz, DMSO-d₆) δ=8.74 (d, J=2.4 Hz, 1H), 8.71 (d, J=2.4 Hz, 1H), 8.34-8.28 (m, 3H), 8.08 (dd, J=8.4, 2.0 Hz, 2H), 7.94 (dd, J=4.8 Hz, 2.0H), 7.51 (d, J=8.4 Hz, 2H), 7.39 (dd, J=8.4, 6.4 Hz, 4H), 7.13 (dd, J=7.6, 1.6 Hz, 1H), 6.49-6.43 (m, 1H), 3.17 (s, 2H), 3.16 (s, 2H), 3.05 (s, 1H), 1.40 (s, 9H).

Step 3. To a solution of tert-butyl 3-(4-(2-(2-aminopyridin-3-yl)-5-phenyl-3H-imidazo[4,5-b]pyridin-3-yl)phenyl) azetidine-1-carboxylate BR2 (200 mg, 0.390 mmol) in DCM (4.00 mL), TFA (2.00 mL) was added at 25° C., and the mixture was stirred for 2 hrs. The reaction mixture was concentrated to give 3-(3-(4-(azetidin-3-yl)phenyl)-5-phenyl-3H-imidazo[4,5-b]pyridin-2-yl)pyridin-2-amine trifluoroacetate BR3 (161 mg, 99.8% yield) as a brown oil, which was used in the next step directly. LCMS Rₜ=0.410 min in 1.0 min chromatography, purity 79.5%, MS ESI calcd, for 418.19 [M+H]⁺ 419.19, found 419.1.

Step 4. To a solution of 3-(3-(4-(azetidin-3-yl)phenyl)-5-phenyl-3H-imidazo[4,5-b]pyridin-2-yl)pyridin-2-amine trifluoroacetate BR3 (100 mg, 0.240 mmol) and 4-(methoxycarbonyl)benzoic acid (43.0 mg, 0.240 mmol) in DMF (5.00 mL), DIEA (93.0 mg, 0.720 mmol) and HATU (136 mg, 0.360 mmol) were added and the mixture was stirred at 25° C. for 2 hrs. Water (2 mL) was added and the resulting mixture was extracted with EtOAc (5 mL×3). The combined organic phase was washed with brine (10 mL), dried over anhydrous Na₂SO₄, filtered and concentrated. The residue was purified by prep-HPLC (Column: Welch Xtimate C₁₈ 150*30 mm*5 μm; mobile phase: [water (NH₄HCO₃)-ACN]; B %: 26%-66%; 20 min) to afford methyl 4-(3-(4-(2-(2-aminopyridin-3-yl)-5-phenyl-3H-imidazo[4,5-b]pyridin-3-yl)phenyl) azetidine-1-carbonyl)benzoate 69 (15.0 mg, 10.7% yield) as a light yellow solid. ¹H NMR (DMSO-d₆, 400 MHz) δ_{H}=8.27 (d, J=8.4 Hz, 1H), 8.05-7.97 (m, 6H), 7.84 (d, J=8.4 Hz, 2H), 7.61 (d, J=8.4 Hz, 2H), 7.53-7.43 (m, 4H), 7.42-7.37 (m, 1H), 7.25-7.19 (m, 1H), 6.94 (s, 2H), 6.43 (dd, J=4.8, 7.6 Hz, 1H), 4.75-4.66 (m, J=8.5, 8.5 Hz, 1H), 4.59-4.43 (m, 2H), 4.18-4.03 (m, 2H), 3.88 (s, 3H). HPLC R; =4.612 min in 8 min chromatography, purity 98.7%. LCMS Rₜ=1.401 min in 2 min chromatography, purity 97.7%, MS ESI calcd, for $80.22, [M+H]⁺ 581.22, found 581.1.

### Example 70. Synthesis of 4-(3-(4-(2-(2-aminopyridin-3-yl)-5-phenyl-3H-imidazo[4,5-b]pyridin-3-yl)phenyl) azetidine-1-carbonyl)benzoic acid (Compound 70)

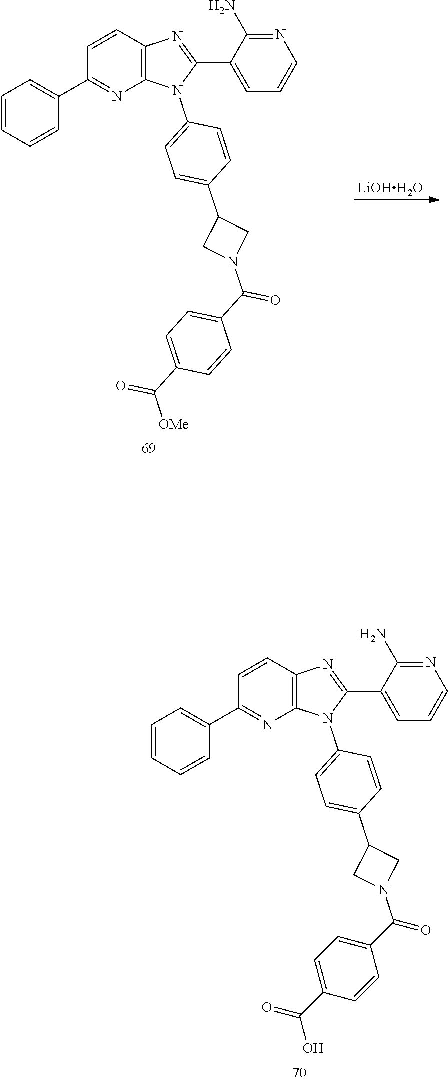 [SMILES: COC(=O)c1ccc(C(=O)N2CC(c3ccc(-n4c(-c5cccnc5N)nc5ccc(-c6ccccc6)nc54)cc3)C2)cc1 ; "LiOH·H2O" ; Nc1ncccc1-c1nc2ccc(-c3ccccc3)nc2n1-c1ccc(C2CN(C(=O)c3ccc(C(=O)O)cc3)C2)cc1]

Step 1. To a solution of methyl 4-(3-(4-(2-(2-aminopyridin-3-yl)-5-phenyl-3H-imidazo[4,5-b]pyridin-3-yl)phenyl) azetidine-1-carbonyl)benzoate 69 (100 mg, 0.170 mmol) in THF (3.00 mL) and water (3.00 mL), LiOH·H₂O (12.0 mg, 0.520 mmol) was added and the mixture was stirred at 25° C. for 12 hrs. The resulting mixture was concentrated and the residue was purified by prep-HPLC (Column: Welch Xtimate C18 150*30 mm*5 μm; mobile phase: [water (NH₃H₂O+NH₄HCO₃)-ACN]; B %: 26%-66%; 20 min) to afford 4-(3-(4-(2-(2-aminopyridin-3-yl)-5-phenyl-3H-imidazo[4,5-b]pyridin-3-yl)phenyl) azetidine-1-carbonyl)benzoic acid 70 (30.7 mg, 31.5% yield) as a white solid. ¹H NMR (DMSO-d₆, 400 MHz) δ_{H}=8.27 (d, J=8.0 Hz, 1H), 8.05-7.96 (m, 6H), 7.80 (d, J=8.4 Hz, 2H), 7.61 (d, J=8.4 Hz, 2H), 7.53-7.44 (m, 4H), 7.42-7.37 (m, 1H), 7.25-7.18 (m, 1H), 6.93 (s, 2H), 6.43 (dd, J=5.2, 7.6 Hz, 1H), 4.76-4.66 (m, 1H), 4.60-4.42 (m, 2H), 4.17-4.03 (m, 2H). HPLC Rₜ=2.004 min in 8 min chromatography, purity 100%. LCMS R (=1.206 min in 2 min chromatography, purity 99.1%, MS ESI calcd, for 566.21, [M+H]⁺ 567.21, found 567.0.

### Example 71. Synthesis of methyl 3-(3-(4-(2-(2-aminopyridin-3-yl)-5-phenyl-3H-imidazo[4,5-b]pyridin-3-yl)phenyl) azetidine-1-carbonyl)benzoate (Compound 71)

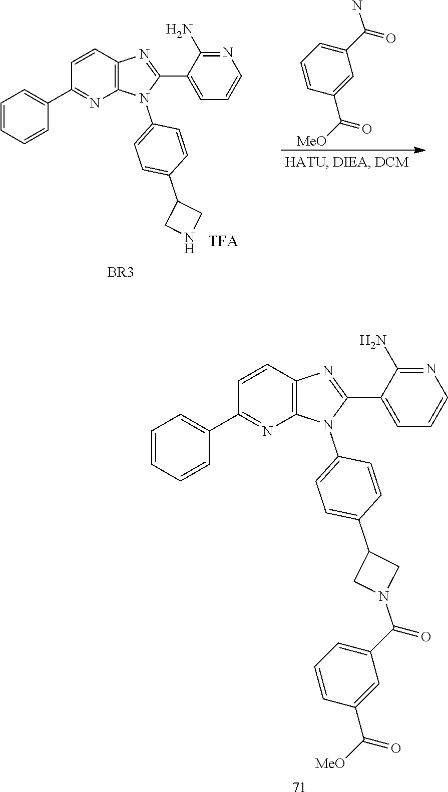 [SMILES: Nc1ncccc1-c1nc2ccc(-c3ccccc3)nc2n1-c1ccc(C2CNC2)cc1 ; COC(=O)c1cccc(C(N)=O)c1 ; "BR3" ; COC(=O)c1cccc(C(=O)N2CC(c3ccc(-n4c(-c5cccnc5N)nc5ccc(-c6ccccc6)nc54)cc3)C2)c1 ; "71"]

Step 1. To a solution of 3-(3-(4-(azetidin-3-yl)phenyl)-5-phenyl-3H-imidazo[4,5-b]pyridin-2-yl)pyridin-2-amine trifluoroacetate BR3 (100 mg, 0.240 mmol) and 3-(methoxy carbonyl)benzoic acid (43 mg, 0.24 mmol) in DMF (5.00 mL), DIEA (93.0 mg, 0.720 mmol) and HATU (136 mg, 0.360 mmol) were added and the mixture was stirred at 25° C. for 2 hrs. Water (2 mL) was added and the resulting mixture was extracted with EtOAc (5 mL×3). The combined organic phase was washed with brine (10 mL), dried over anhydrous Na₂SO₄, filtered and concentrated. The residue was purified by prep-HPLC (Column: Welch Xtimate Cis 150*30 mm*5 μm; mobile phase: [water (NH₄HCO₃)-ACN]; B %: 26%-66%; 20 min) to afford methyl 3-(3-(4-(2-(2-aminopyridin-3-yl)-5-phenyl-3H-imidazo[4,5-b]pyridin-3-yl)phenyl) azetidine-1-carbonyl)benzoate 71 (12.6 mg, 9.08% yield) as a light yellow solid. ¹H NMR (DMSO-d₆, 400 MHz) δ_{H}=8.31-8.22 (m, 2H), 8.11-7.96 (m, 6H), 7.66-7.59 (m, 3H), 7.53-7.44 (m, 4H), 7.42-7.37 (m, 1H), 7.25-7.18 (m, 1H), 6.94 (s, 2H), 6.43 (dd, J=4.8, 7.6 Hz, 1H), 4.79-4.67 (m, 1H), 4.59-4.42 (m, 2H), 4.19-4.04 (m, 2H), 3.89 (s, 3H). HPLC Rₜ=4.628 min in 8 min chromatography, purity 100%. LCMS Rₜ=1.402 min in 2 min chromatography, purity 99.4%, MS ESI calcd, for 580.22, [M+H]⁺ 581.22, found 581.0.

### Example 72. Synthesis of 3-(3-(4-(2-(2-aminopyridin-3-yl)-5-phenyl-3H-imidazo[4,5-b]pyridin-3-yl)phenyl) azetidine-1-carbonyl)benzoic acid (Compound 72)

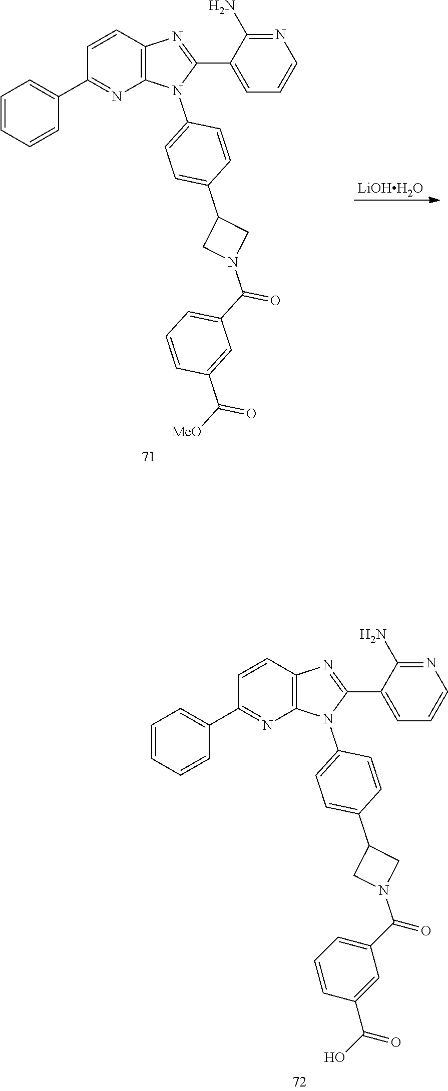 [SMILES: COC(=O)c1cccc(C(=O)N2CC(c3ccc(-n4c(-c5cccnc5N)nc5ccc(-c6ccccc6)nc54)cc3)C2)c1 ; "LiOH·H2O" ; Nc1ncccc1-c1nc2ccc(-c3ccccc3)nc2n1-c1ccc(C2CN(C(=O)c3cccc(C(=O)O)c3)C2)cc1]

Step 1. To a solution of methyl 3-(3-(4-(2-(2-aminopyridin-3-yl)-5-phenyl-3H-imidazo[4,5-b]pyridin-3-yl)phenyl) azetidine-1-carbonyl)benzoate 71 (100 mg, 0.170 mmol) in THF (3.00 mL) and water (3.00 mL). LiOH·H₂O (12.0 mg, 0.520 mmol) was added and the mixture was stirred at 25° C. for 12 hrs. The resulting mixture was concentrated and the residue was purified by prep-HPLC (Column: Welch Xtimate Cis 150*30 mm*5 μm; mobile phase: [water (NH₃H₂O+NH₄HCO₃)-ACN]; B %: 26%-66%; 20 min) to afford 3-(3-(4-(2-(2-aminopyridin-3-yl)-5-phenyl-3H-imidazo[4,5-b]pyridin-3-yl)phenyl) azetidine-1-carbonyl)benzoic acid 72 (29.6 mg, 30.3% yield) as a white solid. ¹H NMR (DMSO-d₆, 400 MHz) δ_{H}=8.30-8.21 (m, 2H), 8.08-7.98 (m, 5H), 7.95-7.90 (m, 1H), 7.63-7.56 (m, 3H), 7.52-7.44 (m, 4H), 7.42-7.37 (m, 1H), 7.24-7.19 (m, 1H), 6.94 (s, 2H), 6.43 (dd, J=4.8, 7.6 Hz, 1H), 4.77-4.70 (m, 1H), 4.59-4.42 (m, 2H), 4.18-4.05 (m, 2H). HPLC Rₜ=2.023 min in 8 min chromatography, purity 100%. LCMS Rₜ=1.232 min in 2 min chromatography, purity 99.1%, MS ESI calcd, for 566.21, [M+H]⁺ 567.21, found 567.1.

### Example 73. Synthesis of methyl 2-(3-(4-(2-(2-aminopyridin-3-yl)-5-phenyl-3H-imidazo[4,5-b]pyridin-3-yl)phenyl) azetidine-1-carbonyl)benzoate (Compound 73)

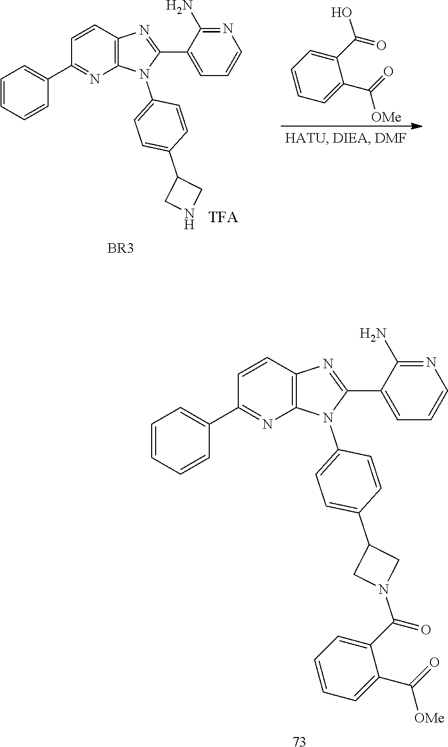 [SMILES: Nc1ncccc1-c1nc2ccc(-c3ccccc3)nc2n1-c1ccc(C2CNC2)cc1 ; COC(=O)c1ccccc1C(=O)O ; "BR3" ; COC(=O)c1ccccc1C(=O)N1CC(c2ccc(-n3c(-c4cccnc4N)nc4ccc(-c5ccccc5)nc43)cc2)C1 ; "73"]

Step 1. To a solution of 3-(3-(4-(azetidin-3-yl)phenyl)-5-phenyl-3H-imidazo[4,5-b]pyridin-2-yl)pyridin-2-amine trifluoroacetate BR3 (100 mg, 0.240 mmol) and 2-(methoxycarbonyl)benzoic acid (43.0 mg, 0.240 mmol) in DMF (5.00 mL), DIEA (93.0 mg, 0.720 mmol) and HATU (136 mg, 0.360 mmol) were added and the mixture was stirred at 25° C. for 2 hrs. Water (2 mL) was added and the resulting mixture was extracted with EtOAc (5 mL×3). The combined organic phase was washed with brine (10 mL), dried over anhydrous Na₂SO₄, filtered and concentrated. The residue was purified by prep-HPLC (Column: Welch Xtimate C18 150*30 mm*5 μm; mobile phase: [water (NH₄HCO₃)-ACN]; B %: 26%-66%; 20 min) to afford methyl 2-(3-(4-(2-(2-aminopyridin-3-yl)-5-phenyl-3H-imidazo[4,5-b]pyridin-3-yl)phenyl) azetidine-1-carbonyl)benzoate 73 (12.6 mg, 9.08% yield) as a light yellow solid. ¹H NMR (DMSO-d₆, 400 MHz) δ_{H}=8.27 (d, J=8.4 Hz, 1H), 8.05-7.97 (m, 4H), 7.89 (d, J=7.6 Hz, 1H), 7.71-7.65 (m, 1H), 7.61-7.55 (m, 3H), 7.53-7.44 (m, 5H), 7.43-7.37 (m, 1H), 7.25-7.18 (m, 1H), 6.94 (s, 2H), 6.42 (dd, J=4.8, 7.6 Hz, 1H), 4.55-4.46 (m, 1H), 4.31-4.23 (m, 1H), 4.13-3.99 (m, 2H), 3.92-3.86 (m, 1H), 3.80 (s, 3H). HPLC Rₜ=4.459 min in 8 min chromatography, purity 100%. LCMS Rₜ=1.364 min in 2 min chromatography, purity 99.2%, MS ESI calcd, for 580.22, [M+H]⁺ 581.22, found 581.0.

### Example 74. Synthesis of 2-(3-(4-(2-(2-aminopyridin-3-yl)-5-phenyl-3H-imidazo[4,5-b]pyridin-3-yl)phenyl) azetidine-1-carbonyl)benzoic acid (Compound 74)

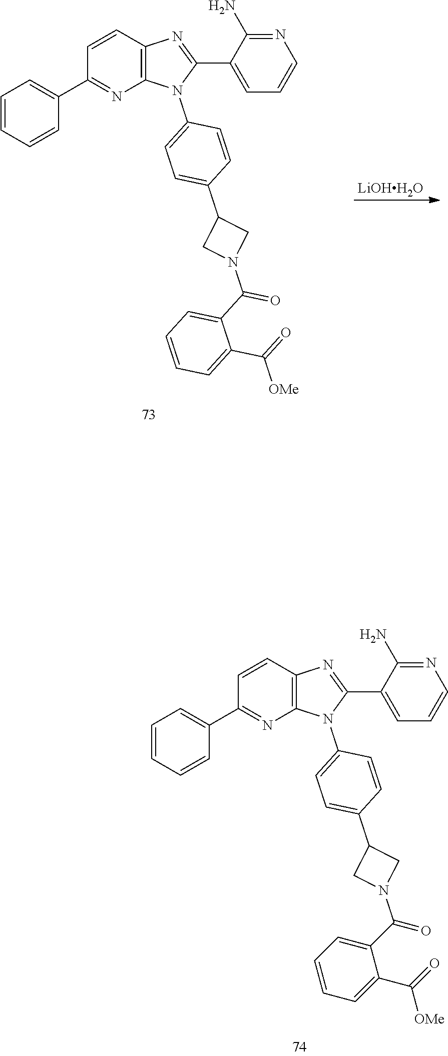 [SMILES: COC(=O)c1ccccc1C(=O)N1CC(c2ccc(-n3c(-c4cccnc4N)nc4ccc(-c5ccccc5)nc43)cc2)C1 ; "LiOH·H2O" ; COC(=O)c1ccccc1C(=O)N1CC(c2ccc(-n3c(-c4cccnc4N)nc4ccc(-c5ccccc5)nc43)cc2)C1]

Step 1. To a solution of methyl 2-(3-(4-(2-(2-aminopyridin-3-yl)-5-phenyl-3H-imidazo[4,5-b]pyridin-3-yl)phenyl) azetidine-1-carbonyl)benzoate 73 (100 mg, 0.170 mmol) in THF (3.00 mL) and water (3.00 mL), LiOH·H₂O (12.0 mg, 0.520 mmol) was added and the mixture was stirred at 25° C. for 12 hrs. The resulting mixture was concentrated and the residue was purified by prep-HPLC (Column: Welch Xtimate C18 150*30 mm*5 μm; mobile phase: [water (NH₃H₂O)+NH₄HCO₃)-ACN]; B %: 26%-66%; 20 min) to afford 2-(3-(4-(2-(2-aminopyridin-3-yl)-5-phenyl-3H-imidazo[4,5-b]pyridin-3-yl)phenyl) azetidine-1-carbonyl)benzoic acid 74 (29.0 mg, 29.7% yield) as a white solid. ¹H NMR (DMSO-d₆, 400 MHz) δ_{H}=8.27 (d, J=8.4 Hz, 1H), 8.06-7.95 (m, 4H), 7.86 (d, J=7.6 Hz, 1H), 7.62 (d, J=8.0 Hz, 2H), 7.55-7.43 (m, 6H), 7.42-7.36 (m, 1H), 7.34-7.27 (m, 1H), 7.22-7.17 (m, 1H), 6.93 (s, 2H), 6.41 (dd, J=4.8, 7.6 Hz, 1H), 4.45-4.36 (m, 1H), 4.19-4.11 (m, 1H), 4.03-3.87 (m, 3H). HPLC Rₜ=2.155 min in 8 min chromatography, purity 100%. LCMS Rₜ=1.252 min in 2 min chromatography, purity 97.8%, MS ESI calcd, for 566.21, [M+H]⁺ 567.21, found 567.0.

### Example 75. Synthesis of 4-(3-(4-(2-(2-aminopyridin-3-yl)-5-phenyl-3H-imidazo[4,5-b]pyridin-3-yl)phenyl) azetidine-1-carbonyl)-3-methylbenzoic acid (Compound 75)

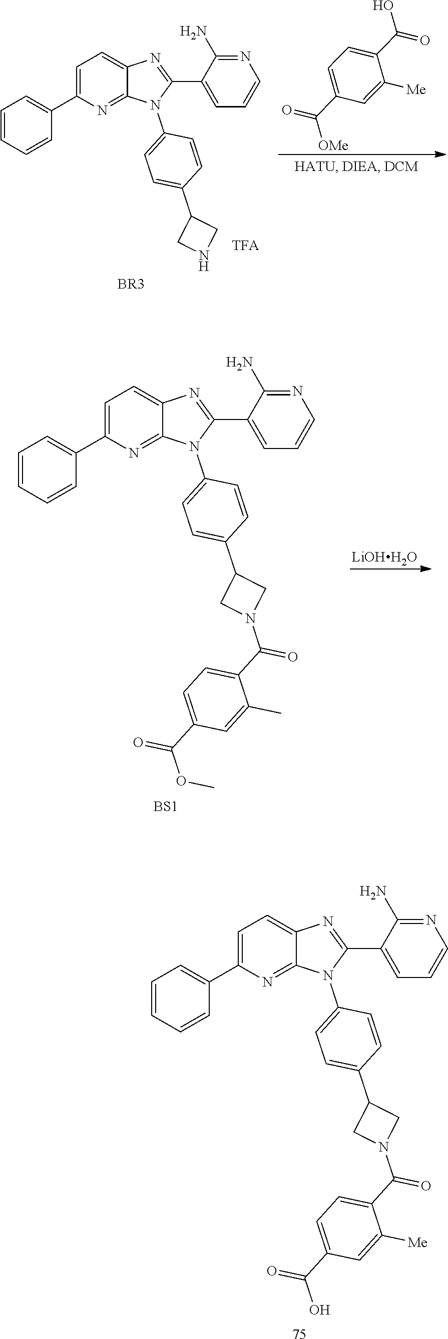

Step 1. To a solution of 3-(3-(4-(azetidin-3-yl)phenyl)-5-phenyl-3H-imidazo[4,5-b]pyridin-2-yl)pyridin-2-amine trifluoroacetate BR3 (100 mg, 0.240 mmol) in DCM (3.00 mL), 4-(methoxycarbonyl)-2-methylbenzoic acid (93.0 mg, 0.480 mmol), DIEA (93.0 mg, 0.720 mmol) and HATU (136 mg, 0.360 mmol) were added at 25° C. and the mixture was stirred for 3 hrs. The reaction mixture was concentrated directly and the resulting residue was purified by flash silica gel chromatography (eluent with 0-3% of MeOH/DCM @ 30 mL/min) to give methyl 4-(3-(4-(2-(2-aminopyridin-3-yl)-5-phenyl-3H-imidazo[4,5-b]pyridin-3-yl)phenyl) azetidine-1-carbonyl)-3-methylbenzoate BS1 (140 mg, 98.5% yield) as a yellow solid. ¹H NMR (400 MHz, DMSO-d₆) δ=8.27 (d, J=8.4 Hz, 1H), 8.06-7.96 (m, 4H), 7.88 (s, 1H), 7.81 (d, J=8.0 Hz, 1H), 7.61-7.43 (m, 7H), 7.43-7.37 (m, 1H), 7.21 (dd, J=7.6, 1.6 Hz, 1H), 6.94 (s, 2H), 6.42 (dd, J=7.6, 4.8 Hz, 1H), 4.58-4.49 (m, 1H), 4.30 (t, J=8.0 Hz, 1H), 4.16-4.10 (m, 1H), 4.08-3.96 (m, 2H), 3.86 (s, 3H), 2.41 (s, 3H).

Step 2. To a solution of methyl 4-(3-(4-(2-(2-aminopyridin-3-yl)-5-phenyl-3H-imidazo[4,5-b]pyridin-3-yl)phenyl) azetidine-1-carbonyl)-3-methylbenzoate BS1 (140 mg, 0.235 mmol) in THE (2.00 mL) and water (2.00 mL), LiOH·H₂O (19.0 mg, 0.471 mmol) was added at 25° C. and the mixture was stirred for 16 hrs. The reaction mixture was concentrated to remove THF and the resulting residue was purified by prep-HPLC (Column: Xtimate C18 150*40 mm*10 μm; Condition: water (HCl)-ACN; begin b: 6-46%) to give 4-(3-(4-(2-(2-aminopyridin-3-yl)-5-phenyl-3H-imidazo[4,5-b]pyridin-3-yl)phenyl) azetidine-1-carbonyl)-3-methylbenzoic acid 75 (47.1 mg, 33.97% yield) as a yellow solid. ¹H NMR (400 MHz, DMSO-d₆) δ=8.31 (d, J=8.4 Hz, 1H), 8.08-7.99 (m, 4H), 7.86 (s, 1H), 7.79 (d, J=7.6 Hz, 1H), 7.72-7.35 (m, 11H), 6.69-6.63 (m, 1H), 4.58-4.49 (m, 1H), 4.35-4.27 (m, 1H), 4.15-3.98 (m, 3H), 2.40 (s, 3H). HPLC Rₜ=4.302 min in 8 min chromatography, purity 98.6%. LCMS Rₜ=1.982 min in 4 min chromatography, purity 99.5%, MS ESI calcd, for 580.22 [M+H]⁺ 581.22, found 581.5.

### Example 76. Synthesis of 4-(3-(4-(2-(2-aminopyridin-3-yl)-5-phenyl-3H-imidazo[4,5-b]pyridin-3-yl)phenyl) azetidine-1-carbonyl)-3-fluorobenzoic acid (Compound 76)

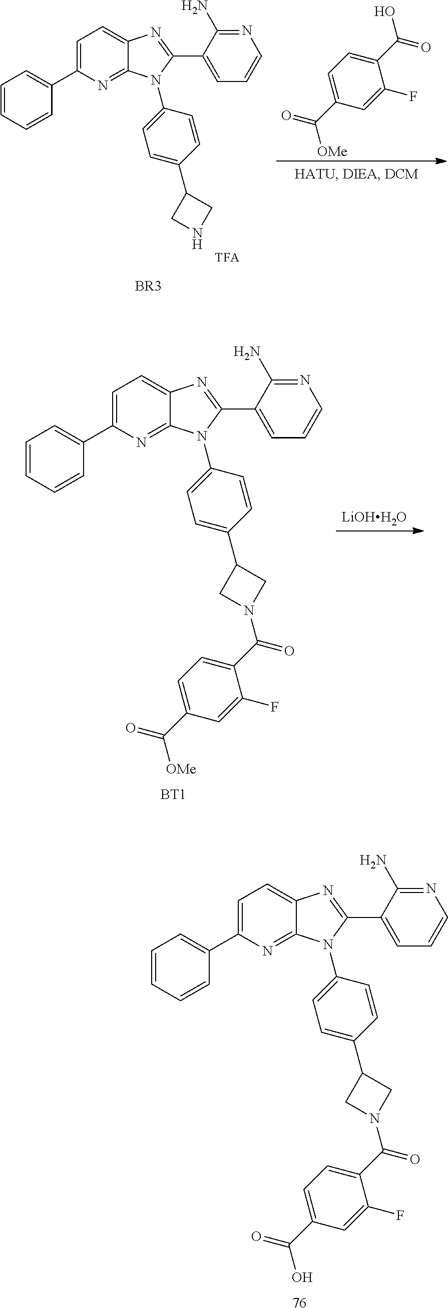

Step 1. To a solution of 3-(3-(4-(azetidin-3-yl)phenyl)-5-phenyl-3H-imidazo[4,5-b]pyridin-2-yl)pyridin-2-amine trifluoroacetate BR3 (100 mg, 0.240 mmol) in DCM (3.00 mL), 2-fluoro-4-(methoxycarbonyl)benzoic acid (95.0 mg, 0.480 mmol), DIEA (93.0 mg, 0.720 mmol) and HATU (136 mg, 0.360 mmol) were added at 25° C. and the mixture stirred for 3 hrs. The reaction mixture was concentrated directly and the resulting residue was purified by flash silica gel chromatography (eluent with 0-3% of MeOH/DCM (@), 30 mL/min) to give methyl 4-(3-(4-(2-(2-aminopyridin-3-yl)-5-phenyl-3H-imidazo[4,5-b]pyridin-3-yl)phenyl) azetidine-1-carbonyl)-3-fluorobenzoate BT1 (122 mg, 85.3% yield) as a yellow oil. ¹H NMR (400 MHz, DMSO-d₆) δ=8.27 (d, J=8.4 Hz, 1H), 8.05-7.97 (m, 5H), 7.82 (d, J=6.0 Hz, 1H), 7.60-7.55 (m, 2H), 7.53-7.43 (m, 4H), 7.42-7.35 (m, 2H), 7.24-7.18 (m, 1H), 6.93 (s, 2H), 6.42 (dd, J=7.2 Hz, 4.8H), 4.59-4.50 (m, 1H), 4.45 (t, J=8.4 Hz, 1H), 4.18-4.11 (m, 3H), 3.87 (s, 3H).

Step 2. To a solution of methyl 4-(3-(4-(2-(2-aminopyridin-3-yl)-5-phenyl-3H-imidazo[4,5-b]pyridin-3-yl)phenyl) azetidine-1-carbonyl)-3-fluorobenzoate BT1 (122 mg, 0.200 mmol) in THF (2.00 mL) and water (2.00 mL), LiOH·H₂O (17.0 mg, 0.410 mmol) was added at 25° C. and the mixture was stirred for 16 hrs. The reaction mixture was concentrated to remove THE and the resulting residue was purified by prep-HPLC (Column: Xtimate Cis 150*40 mm*10 μm; Condition: water (HCl)-ACN; begin b: 6-46%) to give 4-(3-(4-(2-(2-aminopyridin-3-yl)-5-phenyl-3H-imidazo[4,5-b]pyridin-3-yl)phenyl) azetidine-1-carbonyl)-3-fluorobenzoic acid 76 (8.50 mg, 7.05% yield) as a light yellow solid. ¹H NMR (400 MHz, DMSO-d₆) δ=8.36 (d, J=8.4 Hz, 1H), 8.12 (d, J=6.0 Hz, 1H), 8.09-8.03 (m, 3H), 7.91-7.82 (m, 2H), 7.79-7.70 (m, 2H), 7.59 (s, 4H), 7.52-7.39 (m, 3H), 6.90 (t, J=6.8 Hz, 1H), 4.58-4.51 (m, 1H), 4.49-4.42 (m, 1H), 4.18-4.09 (m, 3H). HPLC Rₜ=4.347 min in 8 min chromatography, purity 98.9%. LCMS Rₜ=2.012 min in 4 min chromatography, purity 99.3%, MS ESI calcd, for 584.20 [M+H]⁺ 585.20, found 585.5.

### Example 77. Synthesis of 3-(3-(4-(2-(2-aminopyridin-3-yl)-5-phenyl-3H-imidazo[4,5-b]pyridin-3-yl)phenyl) azetidine-1-carbonyl)-2-methylbenzoic acid (Compound 77)

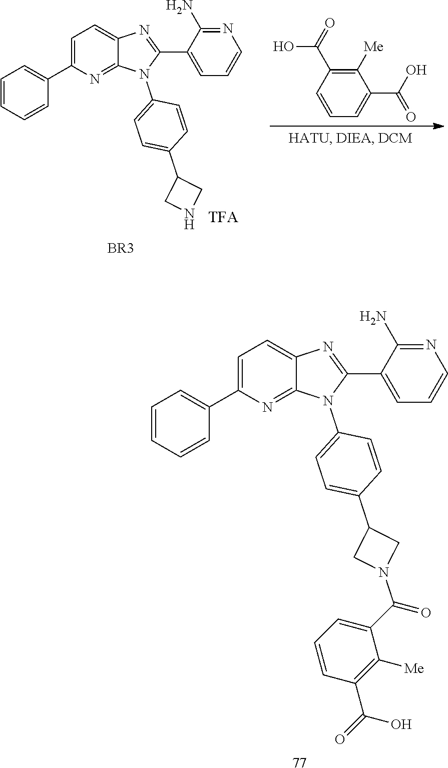 [SMILES: Nc1ncccc1-c1nc2ccc(-c3ccccc3)nc2n1-c1ccc(C2CNC2)cc1 ; Cc1c(C(=O)O)cccc1C(=O)O ; "BR3" ; Cc1c(C(=O)O)cccc1C(=O)N1CC(c2ccc(-n3c(-c4cccnc4N)nc4ccc(-c5ccccc5)nc43)cc2)C1 ; "77"]

Step 1. To a solution of 3-(3-(4-(azetidin-3-yl)phenyl)-5-phenyl-3H-imidazo[4,5-b]pyridin-2-yl)pyridin-2-amine trifluoroacetate BR3 (161 mg, 0.380 mmol) in DCM (3.00 mL), 2-methylisophthalic acid (62.0 mg, 0.350 mmol), DIEA (149 mg, 1.15 mmol) and HATU (219 mg, 0.580 mmol) were added at 25° C. and the mixture was stirred for 16 hrs. The reaction mixture was concentrated directly and the resulting residue was purified by prep-HPLC (column: Xtimate Cis 150*40 mm*10 μm; condition: [water (HCl)-ACN]; begin b: 12-52%) and 2^{nd} prep-HPLC (column: Xtimate C18 150*40 mm*10 μm; condition: water (NH₄HCO₃)-ACN; begin b: 4-44%) to give 3-(3-(4-(2-(2-aminopyridin-3-yl)-5-phenyl-3H-imidazo[4,5-b]pyridin-3-yl)phenyl) azetidine-1-carbonyl)-2-methylbenzoic acid 77 (40.2 mg, 27.4% yield) as a yellow solid. ¹H NMR (400 MHz, DMSO-d₆) δ=8.27 (d, J=8.4 Hz, 1H), 8.06-7.96 (m, 4H), 7.76 (d, J=7.6 Hz, 1H), 7.60-7.54 (m, 2H), 7.52-7.44 (m, 5H), 7.42-7.37 (m, 1H), 7.32 (t, J=8.0 Hz, 1H), 7.20 (dd, J=7.6, 1.6 Hz, 1H), 6.93 (s, 2H), 6.42 (dd, J=7.6, 4.8 Hz, 1H), 4.53 (t, J=9.2 Hz, 1H), 4.30-4.23 (m, 1H), 4.12 (dd, J=9.6, 6.0 Hz, 1H), 4.08-3.99 (m, 1H), 3.94 (dd, J=8.0, 6.4 Hz, 1H), 2.48 (s, 3H). HPLC Rₜ=2.064 min in 8 min chromatography, purity 100%. LCMS Rₜ=0.550 min in 2.5 min chromatography, purity 99.9%, MS ESI calcd, for 580.22 [M+H]⁺ 580.22, found 581.3.

### Example 78. Synthesis of 3-(3-(4-(2-(2-aminopyridin-3-yl)-5-phenyl-3H-imidazo[4,5-b]pyridin-3-yl)phenyl) azetidine-1-carbonyl)-2-fluorobenzoic acid (Compound 78)

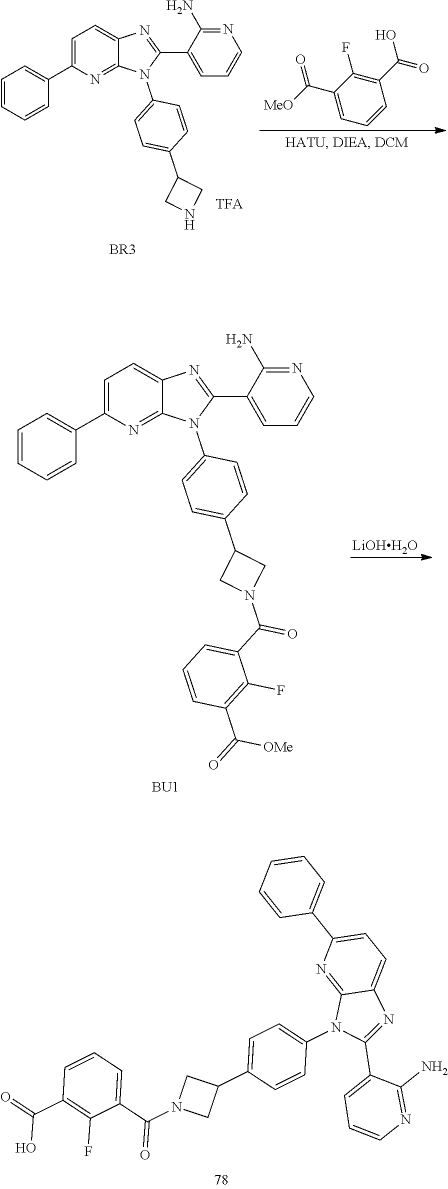

Step 1. To a solution of 3-(3-(4-(azetidin-3-yl)phenyl)-5-phenyl-3H-imidazo[4,5-b]pyridin-2-yl)pyridin-2-amine trifluoroacetate BR3 (100 mg, 0.240 mmol) in DCM (3.00 mL), 2-fluoro-3-(methoxycarbonyl)benzoic acid (95.0 mg, 0.480 mmol), DIEA (93.0 mg, 0.720 mmol) and HATU (136 mg, 0.360 mmol) were added at 25° C. and the mixture was stirred for 3 hrs. The reaction mixture was concentrated directly and the resulting residue was purified by flash silica gel chromatography (eluent with 0-3% of MeOH/DCM @ 30 mL/min) to give methyl 3-(3-(4-(2-(2-aminopyridin-3-yl)-5-phenyl-3H-imidazo[4,5-b]pyridin-3-yl)phenyl) azetidine-1-carbonyl)-2-fluorobenzoate BU1 (50.0 mg, 35.0% yield) as a yellow oil. ¹H NMR (400 MHz, DMSO-d₆) δ=8.27 (d, J=8.4 Hz, 1H), 8.07-7.95 (m, 5H), 7.88-7.81 (m, 1H), 7.61-7.54 (m, 2H), 7.53-7.38 (m, GH), 7.21 (dd, J=7.6, 1.6 Hz, 1H), 6.94 (s, 2H), 6.43 (dd, J=7.6, 4.8 Hz, 1H), 4.58-4.50 (m, 1H), 4.47-4.40 (m, 1H), 4.17-4.06 (m, 3H), 3.87 (s, 3H).

Step 2. To a solution of methyl 3-(3-(4-(2-(2-aminopyridin-3-yl)-5-phenyl-3H-imidazo[4,5-b]pyridin-3-yl)phenyl) azetidine-1-carbonyl)-2-fluorobenzoate BU1 (50.0 mg, 0.0800 mmol) in THF (2.00 mL) and water (2.00 mL), LiOH·H₂O (6.80 mg, 0.170 mmol) was added at 25° C. and the mixture was stirred for 16 hrs. The reaction mixture was concentrated to remove THE and the resulting residue was purified by prep-HPLC (Column: Xtimate C18 150*40 mm*10 μm; Condition: water (HCl)-ACN; begin B: 6-46%) to give 3-(3-(4-(2-(2-aminopyridin-3-yl)-5-phenyl-3H-imidazo[4,5-b]pyridin-3-yl)phenyl) azetidine-1-carbonyl)-2-fluorobenzoic acid 78 (24.9 mg, 50.5% yield) as a light yellow solid. ¹H NMR (400 MHz, DMSO-d₆) δ=8.34 (d, J=8.4 Hz, 1H), 8.20-8.01 (m, 5H), 7.99-7.94 (m, 1H), 7.83-7.71 (m, 2H), 7.63-7.54 (m, 4H), 7.52-7.45 (m, 2H), 7.44-7.36 (m, 2H), 6.80 (s, 1H), 4.54 (t, J=9.2 Hz, 1H), 4.48-4.41 (m, 1H), 4.18-4.01 (m, 3H). HPLC Rₜ=4.302 min in 8 min chromatography, purity 99.0%. LCMS Rₜ=1.954 min in 4 min chromatography, purity 99.5%, MS ESI calcd, for 584.20 [M+H]⁺ 585.20, found 585.5.

### Example 79. Synthesis of 2-(3-((3-(4-(2-(2-aminopyridin-3-yl)-5-phenyl-3H-imidazo[4,5-b]pyridin-3-yl)phenyl) azetidin-1-yl)methyl)phenyl) acetic acid (Compound 79)

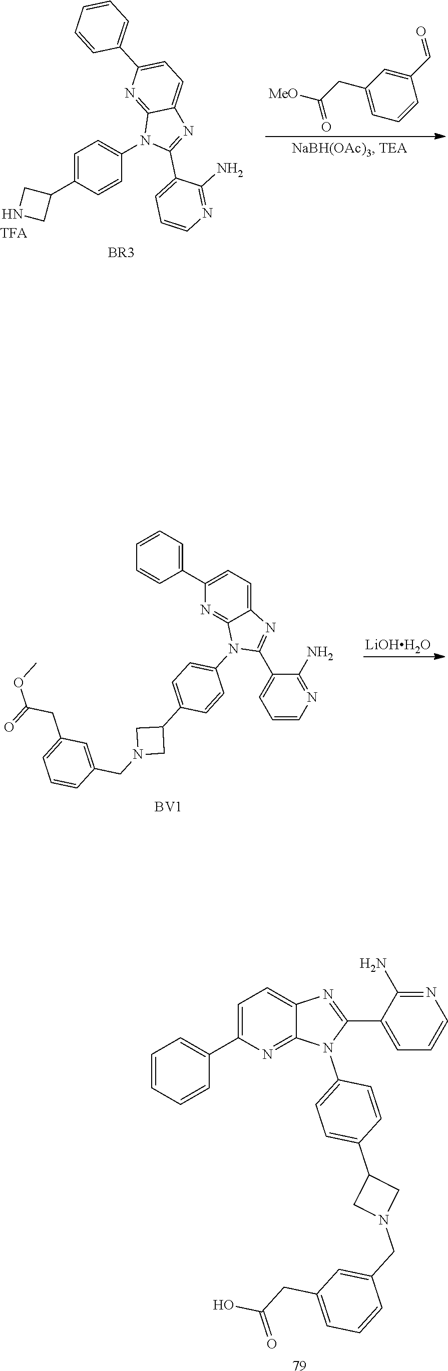

Step 1. To a solution of 3-(3-(4-(azetidin-3-yl)phenyl)-5-phenyl-3H-imidazo[4,5-b]pyridin-2-yl)pyridin-2-amine trifluoroacetate BR3 (180 mg, 0.430 mmol) and methyl 2-(3-formylphenyl)acetate (84.0 mg, 0.470 mmol) in DCM (10.0 mL), TEA (131 mg, 1.29 mmol) and NaBH(OAc)₃ (137 mg, 0.640 mmol) were added and the mixture was stirred at 25° C. for 2 hrs. Water (5 mL) was added to the reaction mixture and the resulting mixture was extracted with DCM (5 mL×3). The combined organic phase was washed with brine (10 mL), dried over anhydrous Na₂SO₄, filtered and concentrated. The residue was purified by flash silica gel chromatography (eluent with 0-6% of MeOH in DCM) to give methyl 2-(3-((3-(4-(2-(2-aminopyridin-3-yl)-5-phenyl-3H-imidazo[4,5-b]pyridin-3-yl)phenyl) azetidin-1-yl)methyl)phenyl)acetate BV1 (240 mg, 76.6% yield) as a light yellow solid. ¹H NMR (DMSO-d₆, 400 MHz) δ_{H}=8.27 (d, J=8.4 Hz, 1H), 8.05-7.98 (m, 4H), 7.62-7.56 (m, 2H), 7.52-7.44 (m, 4H), 7.42-7.34 (m, 4H), 7.31-7.25 (m, 1H), 7.25-7.21 (m, 1H), 6.90 (s, 2H), 6.43 (dd, J=4.8, 7.6 Hz, 1H), 4.15-4.10 (m, 4H), 3.70 (s, 2H), 3.61 (s, 3H), 3.43-3.41 (m, 3H).

Step 2. To a solution of methyl 2-(3-((3-(4-(2-(2-aminopyridin-3-yl)-5-phenyl-3H-imidazo[4,5-b]pyridin-3-yl)phenyl) azetidin-1-yl)methyl)phenyl)acetate BV1 (200 mg, 0.340 mmol) in THF (3.00 mL) and water (3.00 mL), LiOH H₂O (72.0 mg, 1.72 mmol) was added and the mixture was stirred at 25° C. for 4 hrs. The reaction mixture was concentrated directly and the residue was purified by prep-HPLC (Column: Welch Xtimate Cis 150*30 mm*5 μm; mobile phase: [water (FA)-ACN]; B %: 14%-54%; 25 min) to give 2-(3-((3-(4-(2-(2-aminopyridin-3-yl)-5-phenyl-3H-imidazo[4,5-b]pyridin-3-yl)phenyl) azetidin-1-yl)methyl)phenyl) acetic acid 79 (128.8 mg, 64.9% yield) as a light yellow solid. ¹H NMR (DMSO-d₆, 400 MHz) δ_{H}=8.26 (d, J=8.4 Hz, 1H), 8.05-7.96 (m, 4H), 7.55-7.51 (m, 2H), 7.49-7.43 (m, 4H), 7.42-7.37 (m, 1H), 7.28-7.24 (m, 1H), 7.23-7.18 (m, 3H), 7.15-7.11 (m, 1H), 6.95 (s, 2H), 6.42 (dd, J=4.8, 7.6 Hz, 1H), 3.77-3.70 (m, 1H), 3.69-3.63 (m, 4H), 3.55 (s, 2H), 3.20 (t, J=6.4 Hz, 2H). HPLC Rₜ=2.018 min in 8 min chromatography, purity 98.3%. LCMS Rₜ=1.037 min in 2 min chromatography, purity 98.1%, MS ESI calcd. for 566.67, [M+H]⁺ 567.67, found 567.0.

### Example 80. Synthesis of 2-(3-((4-(5-(4-acetamidophenyl)-2-(2-aminopyridin-3-yl)-3H-imidazo[4,5-b]pyridin-3-yl)benzyl)carbamoyl)phenyl) acetic acid (Compound 80)

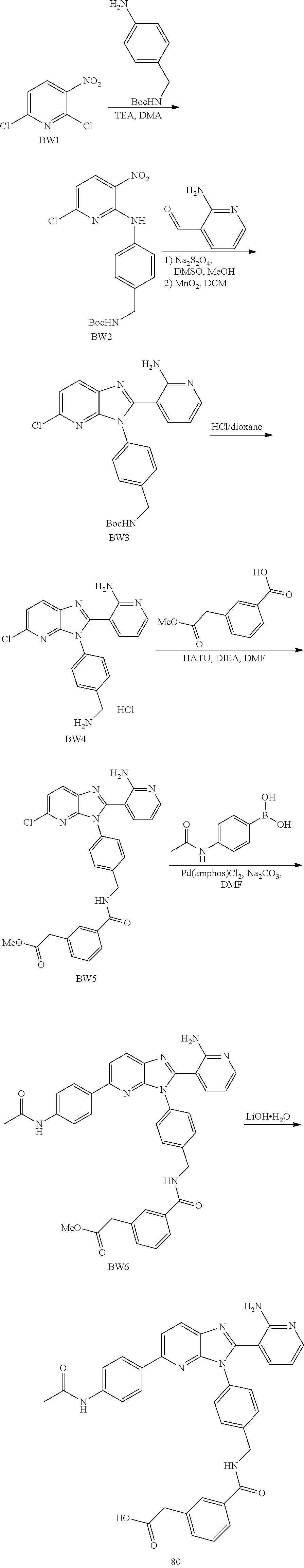

Step 1. To a solution of 2,6-dichloro-3-nitropyridine BW1 (10.0 g, 51.8 mmol) in DMA (50.0 mL), TEA (5.24 g, 51.8 mmol) was added. A mixture of tert-butyl N-[(4-aminophenyl)methyl]carbamate (11.5 g, 51.8 mmol) in DMA (50.0 mL) was then added to the mixture at 0° C., and the mixture was stirred at 0° C. for 1 hr, and subsequently stirred at 25° C. for 11 hrs. The reaction mixture was then diluted with water (250 mL) and extracted with ethyl acetate (200 mL×3). The organic layers were combined and washed with saturated aqueous NaHCO₃ (250 mL×2), and brine (200 mL×5). The organics were dried over Na₂SO₄, filtered and concentrated in vacuo to give tert-butyl (4-((6-chloro-3-nitropyridin-2-yl)amino)benzyl)carbamate BW2 (16.0 g) as a yellow solid. ¹H NMR (400 MHz, DMSO-d₆) δ_{H}=7.60-7.49 (m, 2H), 7.47-7.34 (m, 1H), 7.28-7.21 (m, 2H), 7.09-6.97 (m, 1H), 4.16-4.05 (m, 2H), 1.40 (s, 9H).

Step 2. To a solution of tert-butyl (4-((6-chloro-3-nitropyridin-2-yl)amino)benzyl)carbamate BW2 (2.00 g, 5.28 mmol) and 2-aminopyridine-3-carbaldehyde (0.840 g, 6.86 mmol) in DMSO (20.0 mL) and methanol (20.0 mL), Na₂S₂O₄ (2.30 g. 13.2 mmol) was added and the mixture was stirred at 100° C. for 6 hrs. The reaction mixture was concentrated to remove MeOH, and water (50 mL) was subsequently added to the residue. The resulting mixture was extracted with EtOAc (80 mL×3), and the combined organic phase was washed with saturated LiCl aqueous (80 mL×3), dried over anhydrous Na₂SO₄, filtered and concentrated. The crude product was purified by combi-flash column (EtOAc in PE, 41%) to give the crude product. To a solution of the crude product in DCM (10.0 mL), Mn02 (2.29 g, 26.4 mmol) was added and the mixture was stirred at 35° C. for 12 hrs. The mixture was filtered and washed with MeOH/DCM ( 1/10, 100 mL×5). The filtrate was washed with water (100 mL), dried over anhydrous Na₂SO₄, filtered and concentrated. The reaction mixture was concentrated directly and the crude product was purified by combi-flash column (EtOAc in PE, 50%) to give tert-butyl (4-(2-(2-aminopyridin-3-yl)-5-chloro-3H-imidazo[4,5-b]pyridin-3-yl)benzyl)carbamate BW3 (350 mg, 14.7% yield) as a yellow solid. ÅH NMR (400 MHz, DMSO-d₆) δ_{H}=8.26 (d, J=8.4 Hz, 1H), 7.99 (dd, J=1.6, 4.8 Hz, 1H), 7.50-7.43 (m, 2H), 7.40-7.34 (m, 4H), 7.21 (dd, J=2.0, 7.6 Hz, 1H), 6.91 (s, 2H), 6.44-6.36 (m, 1H), 4.19 (dd, J=6.0 Hz, 2H), 1.41 (s, 9H).

Step 3. A solution of tert-butyl (4-(2-(2-aminopyridin-3-yl)-5-chloro-3H-imidazo[4,5-b]pyridin-3-yl)benzyl)carbamate BW3 (400 mg, 0.880 mmol) in HCl/dioxane (5.00 mL) was stirred at 25° C. for 2 hrs. The reaction mixture was concentrated directly to give 3-(3-(4-(aminomethyl)phenyl)-5-chloro-3H-imidazo[4,5-b]pyridin-2-yl)pyridin-2-amine hydrochloride BW4 (360 mg, 76.9% yield) as a yellow solid. LCMS Rₜ=0.400 min in 1.5 min; purity 66.5%, MS ESI calcd, for 350.10, [M+H]⁺ 351.10, found 351.0.

Step 4. To a solution of 3-(3-(4-(aminomethyl)phenyl)-5-chloro-3H-imidazo[4,5-b]pyridin-2-yl)pyridin-2-amine hydrochloride BW4 (50.0 mg, 0.990 mmol) in DMF (3.00 mL), DIEA (387 mg, 2.99 mmol) and 3-(2-methoxy-2-oxo-ethyl)benzoic acid (252 mg, 1.30 mmol) were added. HATU (493 mg, 1.30 mmol) was then added to the mixture, and the mixture was stirred at 25° C. for 2 hrs. The reaction was diluted with water (20 mL) and extracted with EtOAc (20 ml×3). The combined organic phase was washed with brine (30 ml×3), dried over anhydrous Na₂SO₄, filtered and concentrated. The crude product was purified by combi-flash column (EtOAc in PE, 55%) to give methyl 2-(3-((4-(2-(2-aminopyridin-3-yl)-5-chloro-3H-imidazo[4,5-b]pyridin-3-yl)benzyl)carbamoyl)phenyl)acetate BW5 (360 mg, 68.4% yield) as a yellow solid. ¹H NMR (400 MHz, DMSO-d₆) δ_{H}=9.12 (t, J=6.0 Hz, 1H), 8.25 (d, J=8.4 Hz, 1H), 7.99 (dd, J=2.0, 4.8 Hz, 1H), 7.88-7.77 (m, 2H), 7.48-7.39 (m, 7H), 7.24 (dd, J=2.0, 7.6 Hz, 1H), 6.89 (s, 2H), 6.41 (dd, J=4.8, 7.6 Hz, 1H), 4.57 (d, J=6.0 Hz, 2H), 3.76 (s, 2H), 3.62 (s, 3H).

Step 5. To a solution of methyl 2-(3-((4-(2-(2-aminopyridin-3-yl)-5-chloro-3H-imidazo[4,5-b]pyridin-3-yl)benzyl)carbamoyl)phenyl)acetate BW5 (180 mg, 0.340 mmol) in DMF (6.00 mL) and water (0.120 mL), (4-acetamidophenyl) boronic acid (61.0 mg, 0.340 mmol), Na₂CO₃ (65.0 mg, 0.610 mmol) and bis(di-tert-butyl (4-dimethylaminophenyl)phosphine)dichloropalladium (II) (29.0 mg, 0.0400 mmol) were added and the reaction mixture was degassed and purged with N₂ (×3). The mixture was subjected to microwave irradiation at 160° C. for 1 hr. The mixture was subsequently cooled to 25° C., diluted with EtOAc (30 mL), washed with brine (30 mL×3), dried over Na₂SO₄, and then filtered. The filtrate was concentrated, and the resulting residue was purified by flash silica gel chromatography (eluent with 100% of EtOAc in PE) to give methyl 2-(3-((4-(5-(4-acetamidophenyl)-2-(2-aminopyridin-3-yl)-3H-imidazo[4,5-b]pyridin-3-yl)benzyl)carbamoyl)phenyl)acetate BW6 (160 mg, 52.5% yield) as a yellow solid. ¹H NMR (400 MHz, DMSO-d₆) δ_{H}=10.06 (s, 1H), 9.14 (t, J=6.0 Hz, 1H), 8.27-8.18 (m, 1H), 8.05-7.89 (m, 4H), 7.87-7.80 (m, 2H), 7.70-7.60 (m, 2H), 7.51-7.41 (m, 6H), 7.20-7.16 (m, 1H), 6.99-6.88 (m, 2H), 6.49-6.34 (m, 1H), 4.65-4.55 (m, 2H), 3.77 (s, 2H), 3.6-3.59 (m, 3H), 2.09-2.01 (m, 3H).

Step 6. To a solution of methyl 2-(3-((4-(5-(4-acetamidophenyl)-2-(2-aminopyridin-3-yl)-3H-imidazo[4,5-b]pyridin-3-yl)benzyl)carbamoyl)phenyl)acetate BW6 (160 mg, 0.250 mmol) in THF (2.00 mL) and water (1.00 mL), LiOH·H₂O (32.0 mg, 0.760 mmol) was added and the mixture was stirred at 25° C. for 2 hrs. The reaction mixture was diluted with DMF (4.00 mL) and purified by prep-HPLC (column: welch xtimate c18 150*40 mm*10 μm; mobile phase: [water (FA)-ACN]; B %: 0%-38%: 30 min) to give 2-(3-((4-(5-(4-acetamidophenyl)-2-(2-aminopyridin-3-yl)-3H-imidazo[4,5-b]pyridin-3-yl)benzyl)carbamoyl)phenyl) acetic acid 80 (11.9 mg, 7.0% yield) as a yellow solid ¹H NMR (400 MHz, DMSO-d₆) δ_{H}=10.18-10.07 (m, 1H), 9.14 (t, J=6.0 Hz, 1H), 8.25-8.18 (m, 1H), 8.03-7.89 (m, 4H), 7.85-7.90 (m, 2H), 7.68-7.62 (m, 2H), 7.53-7.39 (m, 6H), 7.24-7.18 (m, 1H), 6.99-6.91 (m, 2H), 6.44-6.38 (m, 1H), 4.60 (d, J=5.6 Hz, 2H), 3.63 (s, 2H), 2.06 (s, 3H). HPLC Rₜ=2.108 min in 8 min chromatography, purity 92.2%. LCMS Rₜ=1.761 min in 4 min; Agilent PoroShell 120 EC-C18 2.7 μm 3.0*50 mm; purity 94.4%, MS ESI calcd, for 611.23, [M+H]⁺ 612.23, found 612.3.

### Example 81. Synthesis of 2-(3-((4-(5-(3-acetamidophenyl)-2-(2-aminopyridin-3-yl)-3H-imidazo[4,5-b]pyridin-3-yl)benzyl)carbamoyl)phenyl) acetic acid (Compound 81)

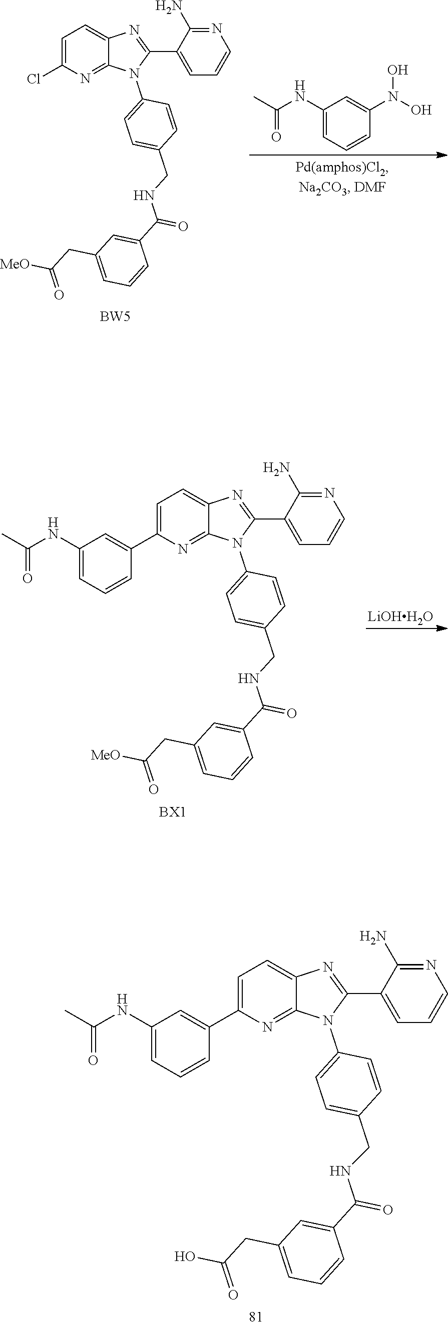

Step 1. To a solution of methyl 2-(3-((4-(2-(2-aminopyridin-3-yl)-5-chloro-3H-imidazo[4,5-b]pyridin-3-yl)benzyl)carbamoyl)phenyl)acetate BW5 (180 mg, 0.340 mmol) in DMF (6.00 mL) and water (0.120 mL), (3-acetamidophenyl) boronic acid (67.0 mg, 0.370 mmol), Na₂CO₃ (54.0 mg, 0.510 mmol) and bis(di-tert-butyl (4-dimethylaminophenyl)phosphine)dichloropalladium (II) (29.0 mg, 0.0400 mmol) were added and the reaction mixture was degassed and purged with N₂ (×3). The mixture was subjected to microwave irradiation at 160° C. for 1 hr. The mixture was subsequently cooled to 25° C., diluted with EtOAc (30 mL), washed with brine (30 mL×3), dried over Na₂SO₄, filtered, and the filtrate concentrated. The residue was purified by flash silica gel chromatography (eluent: 100% of EtOAc in PE) to give methyl 2-(3-((4-(5-(3-acetamidophenyl)-2-(2-aminopyridin-3-yl)-3H-imidazo[4,5-b]pyridin-3-yl)benzyl)carbamoyl)phenyl)acetate BX1 (120 mg, 47.8% yield) as a yellow oil. ¹H NMR (400 MHz, DMSO-d₆) δ_{H}=10.07 (s, 1H), 9.20-9.10 (m, 1H), 8.30-8.25 (m, 1H), 8.08 (s, 1H), 8.02-7.98 (m, 1H), 7.87-7.80 (m, 3H), 7.74-7.69 (m, 1H), 7.67-7.62 (m, 1H), 7.48-7.35 (m, 7H), 7.23-7.16 (m, 1H), 6.89 (s, 2H), 6.41 (dd, J=4.8, 7.6 Hz, 1H), 4.69-4.53 (m, 2H), 3.76 (s, 2H), 3.66-3.59 (m, 3H), 2.04 (s, 3H).

Step 2. To a solution of methyl 2-(3-((4-(5-(3-acetamidophenyl)-2-(2-aminopyridin-3-yl)-3H-imidazo[4,5-b]pyridin-3-yl)benzyl)carbamoyl)phenyl)acetate BX1 (120 mg, 0.190 mmol) in THE (2.00 mL) and water (1.00 mL), LiOH·H₂O (24.0 mg, 0.570 mmol) was added and the mixture was stirred at 25° C. for 2 hrs. The reaction mixture was diluted with DMF (4 mL) and purified by prep-HPLC (column: welch xtimate c18 150*40 mm*10 μm; mobile phase: [water (FA)-ACN]; B %: 0%-40%: 30 min) to give 2-(3-((4-(5-(3-acetamidophenyl)-2-(2-aminopyridin-3-yl)-3H-imidazo[4,5-b]pyridin-3-yl)benzyl)carbamoyl)phenyl) acetic acid 81 (61.9 mg, 50.1% yield) as a yellow solid. ¹H NMR (400 MHz, DMSO-d₆) δ_{H}=10.07 (s, 1H), 9.22-9.10 (m, 1H), 8.30-8.24 (m, 1H), 8.07 (s, 1H), 8.02-7.97 (m, 1H), 7.88-7.79 (m, 3H), 7.74-7.69 (m, 1H), 7.67-7.62 (m, 1H), 7.52-7.42 (m, 7H), 7.40-7.35 (m, 1H), 7.20 (dd, J=1.6, 7.6 Hz, 1H), 6.89 (s, 2H), 6.50-6.31 (m, 1H), 4.60 (d, J=6.0 Hz, 2H), 3.67-3.62 (m, 2H), 2.04 (s, 3H).

### Example 82. Synthesis of 2-(3-((4-(2-(2-aminopyridin-3-yl)-6-phenyl-3H-imidazo[4,5-b]pyridin-3-yl)benzyl)carbamoyl)phenyl) acetic acid (Compound 82)

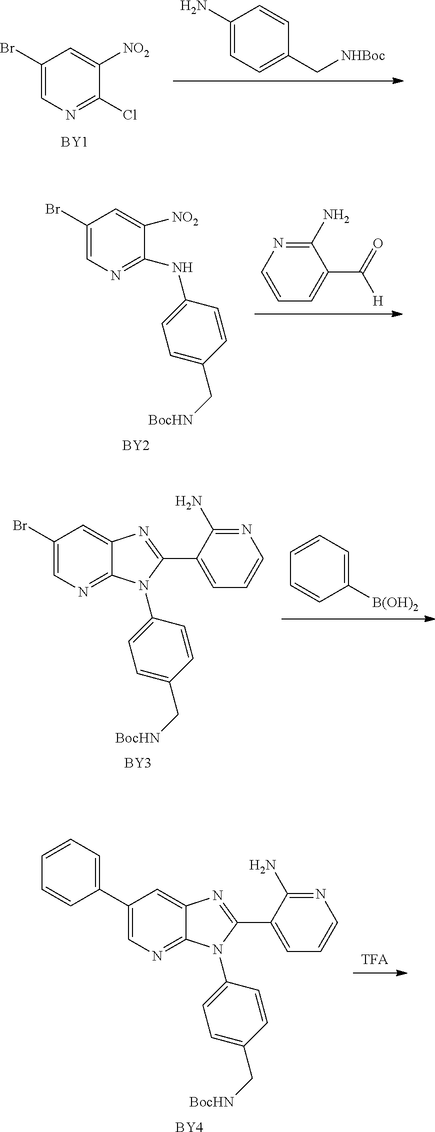

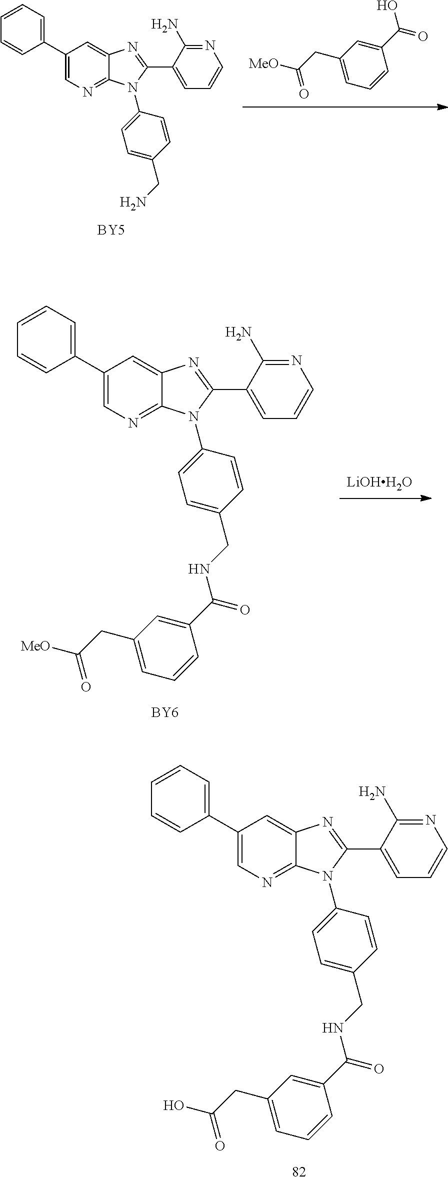

Step 1. To a solution of 5-bromo-2-chloro-3-nitro-pyridine BY1 (1.00 g, 4.21 mmol) in 1,4-dioxane (20.0 mL), tert-butyl N-[(4-aminophenyl)methyl]carbamate (0.940 g, 4.21 mmol) and DIEA (1.10 mL, 6.32 mmol) were added at 25° C., and the mixture stirred at 80° C. for 16 hrs. The reaction mixture was concentrated directly and the resulting residue was purified by flash silica gel chromatography (eluent with 0-15% of EtOAc/PE) to give tert-butyl N-[4-[(5-bromo-3-nitro-2-pyridyl)amino]phenyl]methyl]carbamate BY2 (1.20 g, 2.84 mmol, 67.3% yield) as a red solid. ¹H NMR (400 MHz, DMSO-d₆) δ=9.93 (s, 1H), 8.67 (d, J=2.0 Hz, 1H), 8.58 (d, J=2.4 Hz, 1H), 7.50 (d, J=8.0 Hz, 2H), 7.40 (t, J=6.0 Hz, 1H), 7.22 (d, J=8.4 Hz, 2H), 4.11 (d, J=6.0 Hz, 2H), 1.39 (s, 9H).

Step 2. To a solution of tert-butyl N-[[4-[(5-bromo-3-nitro-2-pyridyl)amino]phenyl]methyl]carbamate BY2 (1.20 g, 2.84 mmol) in DMSO (12.0 mL) and methanol (12.0 mL), 2-aminopyridine-3-carbaldehyde (467 mg, 3.83 mmol) and Na₂S₂O₄ (1.23 g. 7.09 mmol) were added at 25° C., and the mixture was stirred at 100° C. for 16 hrs. The reaction mixture was concentrated, water (10 mL) was added, and the resulting mixture was extracted with EtOAc (10 mL×3). The combined organic phase was washed with brine (10 mL), water (10 mL), dried over anhydrous Na₂SO₄, filtered and concentrated to dryness. The residue was purified by flash silica gel chromatography (eluent with 0-44% of EtOAc in PE) to give tert-butyl N-[[4-[2-(2-amino-3-pyridyl)-6-bromo-imidazo[4,5-b]pyridin-3-yl]phenyl]methyl]carbamate BY3 (666 mg, 1.34 mmol, 47.4% yield) as a yellow oil. ¹H NMR (400 MHz, CHLOROFORM-d) 8=8.91 (d, J=2.0 Hz, 1H), 8.66 (dd, J=4.8, 1.2 Hz, 1H), 8.60 (d, J=2.0 Hz, 1H), 8.28 (d, J=2.0 Hz, 1H), 8.04 (dd, J=5.2, 1.6 Hz, 1H), 7.94 (dt, J=7.6, 1.6 Hz, 1H), 7.50 (d, J=8.4 Hz, 2H), 7.47-7.42 (m, 1H), 7.39 (d, J=8.4 Hz, 2H), 4.99 (s, 1H), 4.44 (d, J=5.6 Hz, 2H), 1.48 (s, 9H).

Step 3. To a solution of tert-butyl (4-(2-(2-aminopyridin-3-yl)-6-bromo-3H-imidazo[4,5-b]pyridin-3-yl)benzyl)carbamate BY3 (400 mg, 0.807 mmol) and phenylboronic acid (197 mg, 1.61 mmol) in 1,4-dioxane (6.00 mL) and water (0.500 mL), K₂CO₃ (335 mg, 2.42 mmol) and Pd(dppf)Cl₂ (33.0 mg, 0.0404 mmol) were added at 25° C., and the mixture was stirred at 90° C. for 16 hrs under N₂. The reaction mixture was concentrated directly, and the resulting residue was purified by flash silica gel chromatography (eluent with 0-5% of MeOH/DCM) to give tert-butyl (4-(2-(2-aminopyridin-3-yl)-6-phenyl-3H-imidazo[4,5-b]pyridin-3-yl)benzyl)carbamate BY4 (316 mg, 0.642 mmol, 79.5% yield) as a brown oil. ¹H NMR (400 MHz, DMSO-d₆) δ=8.63-8.49 (m, 1H), 8.48-8.39 (m, 1H), 8.02-7.98 (m, 1H), 7.78 (d, J=7.6 Hz, 2H), 7.55-7.36 (m, 8H), 7.26-7.21 (m, 1H), 7.07-6.92 (m, 2H), 6.44-6.37 (m, 1H), 4.24-4.18 (m, 2H), 1.40 (s, 9H).

Step 4. To a solution of tert-butyl (4-(2-(2-aminopyridin-3-yl)-6-phenyl-3H-imidazo[4,5-b]pyridin-3-yl)benzyl)carbamate BY4 (300 mg, 0,609 mmol) in DCM (2.00 mL), TFA (2.00 mL) was added at 25° C., and the mixture was stirred for 2 hrs. The reaction mixture was concentrated to give 3-(3-(4-(aminomethyl)phenyl)-6-phenyl-3H-imidazo[4,5-b]pyridin-2-yl)pyridin-2-amine BY5 (300 mg, crude) as a yellow oil. LCMS Rₜ=0.474 min in 1 min chromatography, purity 81.1%, MS ESI calcd, for 392.17 [M+H]⁺ 393.17, found 393.2.

Step 5. To a solution of 3-(3-(4-(aminomethyl)phenyl)-6-phenyl-3H-imidazo[4,5-b]pyridin-2-yl)pyridin-2-amine BY5 (250 mg, 0.905 mmol) in DCM (10.0 mL), 3-(2-methoxy-2-oxo-ethyl)benzoic acid (111 mg, 0.573 mmol), DIEA (148 mg, 1.15 mmol) and HATU (218 mg, 0.573 mmol) were added at 25° C., and the mixture was stirred at for 2 hrs. The reaction mixture was concentrated and the resulting residue was purified by flash silica gel chromatography (eluent with 0-76% of EtOAc/PE) to give methyl 2-(3-((4-(2-(2-aminopyridin-3-yl)-6-phenyl-3H-imidazo[4,5-b]pyridin-3-yl)benzyl)carbamoyl)phenyl)acetate BY6 (200 mg, 0.352 mmol, 92.0% yield) as a brown solid. LCMS Rₜ=0.588 min in 1 min chromatography, purity 81.2%, MS ESI calcd, for 569.22 [M+H]⁺ 569.22, found 569.3

Step 6. To a solution of methyl 2-(3-((4-(2-(2-aminopyridin-3-yl)-6-phenyl-3H-imidazo[4,5-b]pyridin-3-yl)benzyl)carbamoyl)phenyl)acetate BY6 (100 mg, 0.176 mmol) in THF (2.00 mL) and water (2.00 mL), LiOH·H₂O (14.0 mg, 0.352 mmol) was added at 25° C., and the mixture was stirred for 2 hrs. The reaction mixture was concentrated to remove THE. The residue was purified by prep-HPLC (Column: Xtimate C18 150*40 mm*10 μm; condition: water (HCl)-ACN; begin b: 6-46) to give 2-(3-((4-(2-(2-aminopyridin-3-yl)-6-phenyl-3H-imidazo[4,5-b]pyridin-3-yl)benzyl)carbamoyl)phenyl) acetic acid 82 (25.0 mg, 0.0445 mmol, 25.3% yield) as a white solid. ¹H NMR (400 MHz, DMSO-d₆) δ_{H}=9.12 (t, J=6.0 Hz, 1H), 8.61 (d, J=2.0 Hz, TH), 8.45 (d, J=2.0 Hz, TH), 8.00 (d, J=3.2 Hz, TH), 7.82-7.72 (m, 4H), 7.57-7.34 (m, 9H), 7.27 (dd, J=7.6, 1.6 Hz, 1H), 7.02 (s, 2H), 6.43 (dd, J=7.6, 4.8 Hz, 1H), 4.57 (d, J=6.0 Hz, 2H), 3.52 (s, 2H). HPLC Rₜ=3.547 min in 8 min chromatography. purity 99.7%. LCMS R=1.129 min in 2 min chromatography, purity 99.2%, MS ESI calcd, for 554.21 [M+H]⁺ 554.21, found 555.2.

### Example 83. Synthesis of 2-(3-((4-(6-(3-acetamidophenyl)-2-(2-aminopyridin-3-yl)-3H-imidazo[4,5-b]pyridin-3-yl)benzyl)carbamoyl)phenyl) acetic acid (Compound 83)

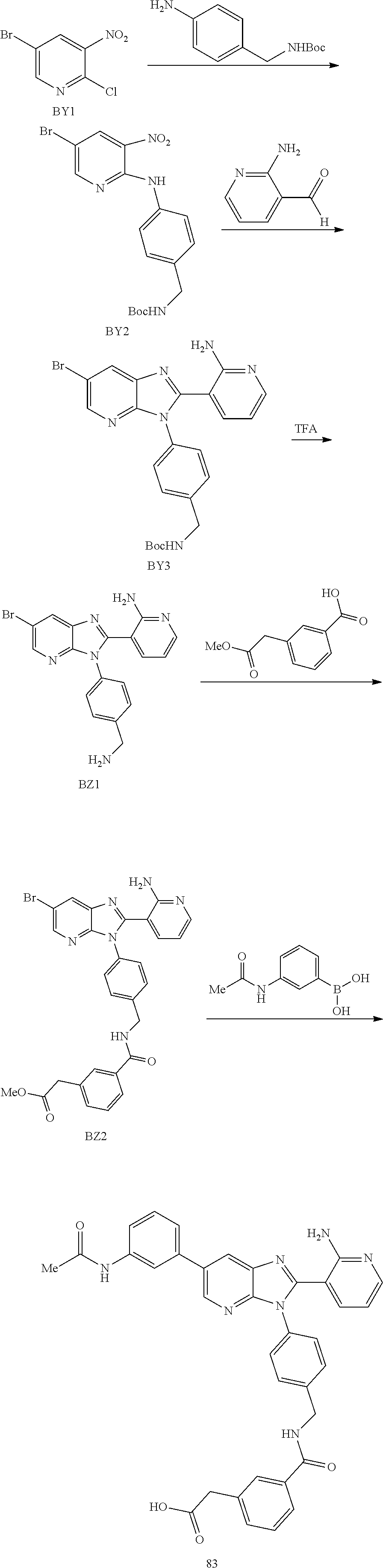

Step 1. To a solution of 5-bromo-2-chloro-3-nitro-pyridine BY1 (1.00 g, 4.21 mmol) in 1,4-dioxane (20.0 mL), tert-butyl N-[(4-aminophenyl)methyl]carbamate (0.940 g, 4.21 mmol) and DIEA (1.10 mL, 6.32 mmol) were added at 25° C., and the mixture was stirred at 80° C. for 16 hrs. The reaction mixture was concentrated directly and the resulting residue was purified by flash silica gel chromatography (eluent with 0-15% of EtOAc/PE) to give tert-butyl N-[4-[(5-bromo-3-nitro-2-pyridyl)amino]phenyl]methyl]carbamate BY2 (1.20 g, 2.84 mmol, 67.3% yield) as a red solid. ¹H NMR (400 MHz, DMSO-d₆) δ=9.93 (s, 1H), 8.67 (d, J=2.0 Hz, 1H), 8.58 (d, J=2.4 Hz, 1H), 7.50 (d, J=8.0 Hz, 2H), 7.40 (t, J=6.0 Hz, 1H), 7.22 (d, J=8.4 Hz, 2H), 4.11 (d, J=6.0 Hz, 2H), 1.39 (s, 9H).

Step 2. To a solution of tert-butyl N-[[4-[(5-bromo-3-nitro-2-pyridyl)amino]phenyl]methyl]carbamate BY2 (1.20 g, 2.84 mmol) in DMSO (12.0 mL) and methanol (12.0 mL), 2-aminopyridine-3-carbaldehyde (467 mg, 3.83 mmol) and Na₂S₂O₄ (1.23 g, 7.09 mmol) were added at 25° C., and the mixture was stirred at 100° C. for 16 hrs. The reaction mixture was concentrated, water (10 mL) was added, and the resulting mixture was extracted with EtOAc (10 mL×3). The combined organic phase was washed with brine (10 mL), water (10 mL), dried over anhydrous Na₂SO₄, filtered and concentrated to dryness. The residue was purified by flash silica gel chromatography (eluent with 0-44% of EtOAc in PE) to give tert-butyl N-[[4-[2-(2-amino-3-pyridyl)-6-bromo-imidazo[4,5-b]pyridin-3-yl]phenyl methyl]carbamate BY3 (666 mg, 1.34 mmol, 47.4% yield) as a yellow oil, ¹H NMR (400 MHz, CHLOROFORM-d) δ=8.91 (d, J=2.0 Hz, 1H), 8.66 (dd, J=4.8, 1.2 Hz, 1H), 8.60 (d, J=2.0 Hz, 1H), 8.28 (d, J=2.0 Hz, 1H), 8.04 (dd, J=5.2, 1.6 Hz, 1H), 7.94 (dt, J=7.6, 1.6 Hz, 1H), 7.50 (d, J=8.4 Hz, 2H), 7.47-7.42 (m, 1H), 7.39 (d, J=8.4 Hz, 2H), 4.99 (s, 1H), 4.44 (d, J=5.6 Hz, 2H), 1.48 (s, 9H).

Step 3. To a solution of tert-butyl (4-(2-(2-aminopyridin-3-yl)-6-bromo-3H-imidazo[4,5-b]pyridin-3-yl)benzyl)carbamate BY3 (600 mg, 1.22 mmol) in DCM (10.0 mL), TFA (1.00 mL) was added and the mixture was stirred at 25° C. for 2 hrs. The reaction mixture was concentrated to give 3-(3-(4-(aminomethyl)phenyl)-6-bromo-3H-imidazo[4,5-b]pyridin-2-yl)pyridin-2-amine BZ1 (470 mg, crude) as a yellow oil, which was used directly in the next step directly.

Step 4. To a solution of 3-(3-(4-(aminomethyl)phenyl)-6-bromo-3H-imidazo[4,5-b]pyridin-2-yl)pyridin-2-amine BZ1 (500 mg, 1.26 mmol) in DMF (3.00 mL), 3-(2-methoxy-2-oxo-ethyl)benzoic acid (246 mg, 1.26 mmol) and DIEA (490 mg, 3.79 mmol) were added. HATU (577 mg, 1.52 mmol) was then added to the mixture, and the mixture was stirred at 25° C. for 2 hrs. The reaction mixture was diluted with water (20 mL) and extracted with EtOAc (30 mL×2). The combined organic phase was washed with brine (30 mL×5), dried over anhydrous Na₂SO₄, filtered and concentrated. The residue was purified by combi-flash column (EtOAc in PE, 69%) to give methyl 2-(3-((4-(2-(2-aminopyridin-3-yl)-6-bromo-3H-imidazo[4,5-b]pyridin-3-yl)benzyl)carbamoyl)phenyl)acetate BZ2 (360 mg, 31.7% yield) as a yellow solid. ¹H NMR (400 MHz, DMSO-d₆) δ_{H}=9.11 (t, J=6.0 Hz, 1H), 8.51-4.48 (m, 1H), 8.41-8.38 (m, 1H), 8.00 (dd, J=2.0, 4.8 Hz, 1H), 7.83-7.80 (m, 2H), 7.45-7.38 (m, 6H), 7.26 (dd, J=1.6, 7.6 Hz, 1H), 7.00-6.80 (m, 2H), 6.43-6.39 (m, 1H), 4.56 (d, J=6.0 Hz, 2H), 3.76 (s, 2H), 3.62 (s, 3H).

Step 5. To a solution of (3-acetamidophenyl) boronic acid (56.0 mg, 0.310 mmol) and methyl 2-(3-((4-(2-(2-aminopyridin-3-yl)-6-bromo-3H-imidazo[4,5-b]pyridin-3-yl)benzyl)carbamoyl)phenyl)acetate BZ2 (180 mg, 0.310 mmol) in 1,4-dioxane (15.0 mL) and water (3.00 mL), Cs₂CO₃ (308 mg, 0.940 mmol) and Pd(dppf)Cl₂ (23.0 mg, 0.0300 mmol) were added, the reaction mixture was degassed and purged with N₂ (×3), and the mixture was stirred at 90° C. for 12 hrs. The reaction mixture was filtered, the filtrate concentrated, and the crude material was diluted with DMF (5.00 mL) and purified by prep-HPLC (Column: Welch Xtimate C18 150*40 mm*10 μm; mobile phase: [water (HCl)-ACN]; B %: 0%-40%; 36 min) to give 2-(3-((4-(6-(3-acetamidophenyl)-2-(2-aminopyridin-3-yl)-3H-imidazo[4,5-b]pyridin-3-yl)benzyl)carbamoyl)phenyl) acetic acid 83 (15.0 mg, 7.6% yield) as a yellow solid. ¹H NMR (400 MHz, DMSO-d₆) δ_{H}=10.17 (s, 1H), 9.16 (t, J=1.6 Hz, 1H), 8.64 (d, J=2.0 Hz, 1H), 8.55-8.36 (m, 2H), 8.12 (dd, J=1.6, 6.4 Hz, 1H), 8.01-7.98 (m, 1H), 7.94 (dd, J=1.6, 7.6 Hz, 1H), 7.86-7.79 (m, 2H), 7.67-7.00 (m, 1H), 7.54-7.48 (m, 4H), 7.47-7.40 (m, 4H), 6.95-6.86 (m, 1H), 4.57 (d, J=6.0 Hz, 2H), 3.65 (s, 2H), 2.08 (s, 3H). HPLC Rₜ=2.132 min in 8 min chromatography, purity 97.9%. LCMS Rₜ=1.320 min in 4 min; Agilent PoroShell 120 EC-C18 2.7 μm 3.0*50 mm; purity 99.1%, MS ESI calcd, for 611.23, [M+H]⁺ 612.23, found 612.2.

### Example 84. Synthesis of 2-(3-((4-(6-(4-acetamidophenyl)-2-(2-aminopyridin-3-yl)-3H-imidazo[4,5-b]pyridin-3-yl)benzyl)carbamoyl)phenyl) acetic acid (Compound 84)

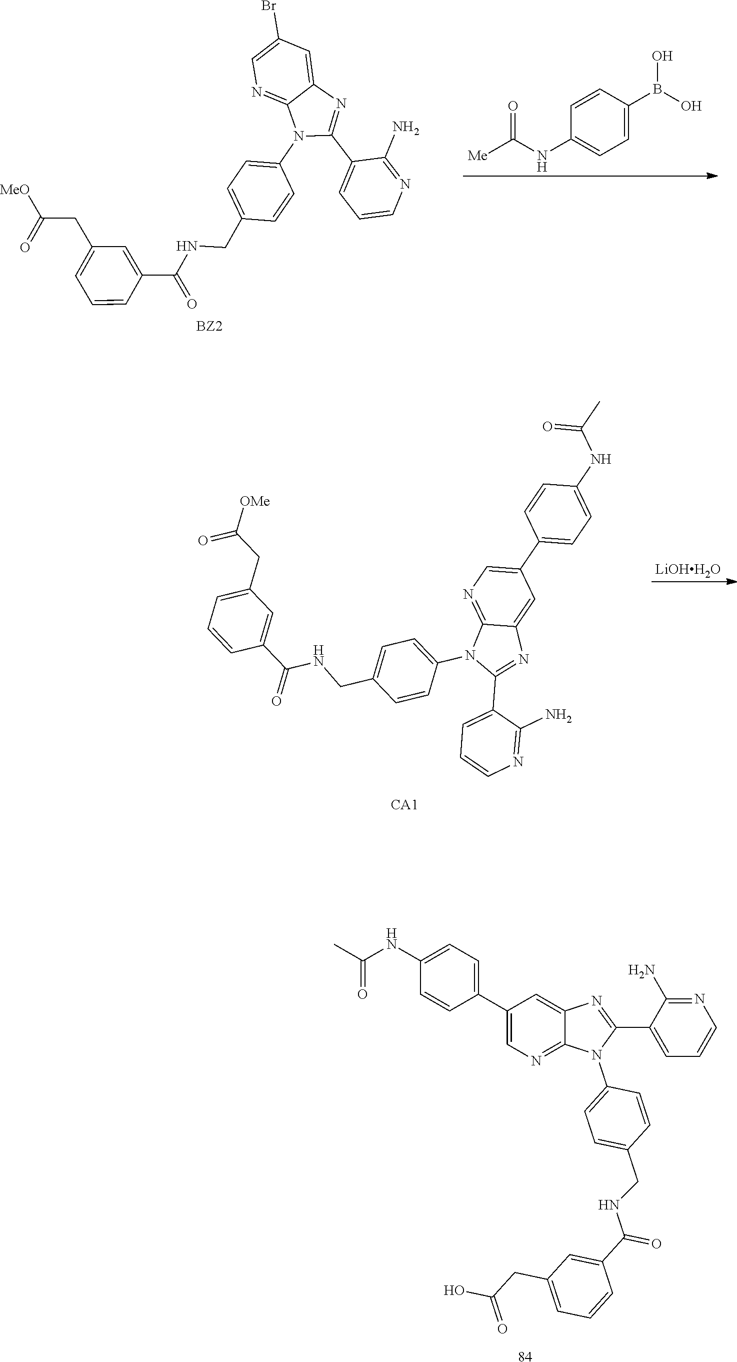

Step 1. To a solution of (4-acetamidophenyl) boronic acid (141 mg, 0.780 mmol) and methyl 2-(3-((4-(2-(2-aminopyridin-3-yl)-6-bromo-3H-imidazo[4,5-b]pyridin-3-yl)benzyl)carbamoyl)phenyl)acetate BZ2 (150 mg, 0.260 mmol) in 1,4-dioxane (5.00 mL) and water (1.50 mL), K₂CO₃ (109 mg, 0.780 mmol) and Pd(dppf)Cl₂ (19.0 mg, 0.0200 mmol) were added, the reaction mixture was degassed and purged with N₂ (×3), and the mixture was stirred at 90° C. for 12 hrs. The reaction mixture was filtered, the filtrate concentrated, and the residue was purified by combi-flash column (EtOAc in PE, 100%) to give methyl 2-(3-((4-(6-(4-acetamidophenyl)-2-(2-aminopyridin-3-yl)-3H-imidazo[4,5-b]pyridin-3-yl)benzyl)carbamoyl)phenyl)acetate CA1 (90.0 mg, 46.6% yield) as a yellow solid. ¹H NMR (400 MHz, DMSO-d₆) δ_{H}=10.06 (s, 1H), 9.20-9.07 (m, 1H), 8.58 (d, J=2.0 Hz, 1H), 8.41 (d, J=2.0 Hz, 1H), 8.00 (dd, J=2.0, 4.8 Hz, 1H), 7.84-7.80 (m, 2H), 7.72-7.70 (m, 4H), 7.54-7.49 (m, 2H), 7.47-7.43 (m, 4H), 7.26 (dd, J=1.6, 7.6 Hz, 1H), 7.02 (s, 2H), 6.42 (dd, J=4.8, 7.6 Hz, 1H), 4.57 (d, J=5.6 Hz, 2H), 3.76 (s, 2H), 3.63 (s, 3H), 2.09 (s, 3H).

Step 2. To a solution of methyl 2-(3-((4-(6-(4-acetamidophenyl)-2-(2-aminopyridin-3-yl)-3H-imidazo[4,5-b]pyridin-3-yl)benzyl)carbamoyl)phenyl)acetate CA1 (90.0 mg, 0.140 mmol) in THF (2.00 mL) and water (1.00 mL), LiOH·H₂O (24.0 mg, 0.570 mmol) was added and the mixture was stirred at 25° C. for 2 hrs. The reaction mixture was diluted with DMF (4 mL) and purified by prep-HPLC (column: welch xtimate c18 150*40 mm*10 μm; mobile phase: [water (HCl)-ACN]; B %: 0%-40%: 36 min) to give 2-(3-((4-(6-(4-acetamidophenyl)-2-(2-aminopyridin-3-yl)-3H-imidazo[4,5-b]pyridin-3-yl)benzyl)carbamoyl)phenyl) acetic acid 84 (24.6 mg, 27.8% yield) as a yellow solid. ¹H NMR (400 MHz, DMSO-d₆) δ_{H}=10.14 (s, 1H), 9.16 (d, J=2.0 Hz, 1H), 8.69 (d, J=2.0 Hz, 1H), 8.55-8.40 (d, J=2.0 Hz, 2H), 8.12 (dd, J=1.6, 6.4 Hz, 1H), 7.93 (dd, J=1.6, 7.6 Hz, 1H), 7.86-7.79 (m, 2H), 7.77-7.70 (m, 4H), 7.53-7.47 (m, 4H), 7.46-7.40 (m, 2H), 6.94-6.87 (m, 1H), 4.57 (d, J=5.6 Hz, 2H), 3.65 (s, 2H), 2.08 (s, 3H). HPLC Rₜ=2.054 min in 8 min chromatography, purity 99.6%. LCMS Rₜ=1.295 min in 4 min: Agilent PoroShell 120 EC-C18 2.7 μm 3.0*50 mm; purity 100%, MS ESI calcd. For 611.23, [M+H]⁺ 612.23, found 612.3.

### Example 85. Synthesis of 2-(3-((4-(2-(2-aminopyridin-3-yl)-6-(3-benzamidophenyl)-3H-imidazo[4,5-b]pyridin-3-yl)benzyl)carbamoyl)phenyl) acetic acid (Compound 85)

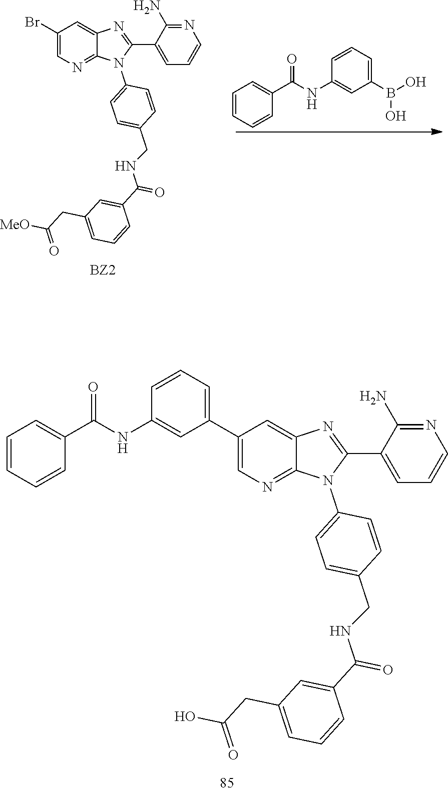

Step 1. To a solution of methyl 2-(3-((4-(2-(2-aminopyridin-3-yl)-6-bromo-3H-imidazo[4,5-b]pyridin-3-yl)benzyl)carbamoyl)phenyl)acetate BZ2 (180 mg, 0.310 mmol) and (3-benzamidophenyl) boronic acid (114 mg, 0.470 mmol) in 1,4-dioxane (15.0 mL) and water (3.00 mL), Cs₂CO₃ (308 mg, 0.940 mmol) and Pd(dppf)Cl₂ (23.0 mg, 0.0300 mmol) were added, the reaction mixture was degassed and purged with N₂ (×3), and the mixture was stirred at 90° C. for 12 hrs. The reaction mixture was filtered, the filtrate was concentrated, and the resulting crude material was diluted with DMF (5 mL) and purified by prep-HPLC (Column: Welch Xtimate C18 150*30 mm*5 μm; mobile phase: [water (HCl)-ACN]; B %: 10%-50%; 36 min) to give 2-(3-((4-(2-(2-aminopyridin-3-yl)-6-(3-benzamidophenyl)-3H-imidazo[4,5-b]pyridin-3-yl)benzyl)carbamoyl)phenyl) acetic acid 85 (28.4 mg, 13.0% yield) as a yellow solid. ¹H NMR (400 MHz, DMSO-d₆) δ_{H}=10.42 (s, 1H), 9.15 (t, J=2.0 Hz, 1H), 8.69 (d, J=2.0 Hz, 1H), 8.49 (d, J=2.0 Hz, 1H), 8.45-8.28 (m, 1H), 8.24-8.19 (m, 1H), 8.11 (dd, J=1.6, 6.0 Hz, 1H), 8.03-7.98 (m, 2H), 7.94-7.90 (m, 1H), 7.89-7.85 (m, 1H), 7.84-7.80 (m, 2H), 7.64-7.59 (m, 1H), 7.58-7.48 (m, 8H), 7.46-7.42 (m, 2H), 6.92-6.85 (m, 1H), 4.57 (d, J=5.6 Hz, 2H), 3.65 (s, 2H). HPLC R; =2.891 min in 8 min chromatography, purity 97.8%. LCMS Rₜ=1.730 min in 4 min; Agilent PoroShell 120 EC-C18 2.7 μm 3.0*50 mm; purity 98.2%, MS ESI calcd, for 673.24, [M+H]⁺ 674.24, found 674.2.

### Example 86. Synthesis of 2-(3-((4-(2-(2-aminopyridin-3-yl)-6-(4-benzamidophenyl)-3H-imidazo[4,5-b]pyridin-3-yl)benzyl)carbamoyl)phenyl) acetic acid (Compound 86)

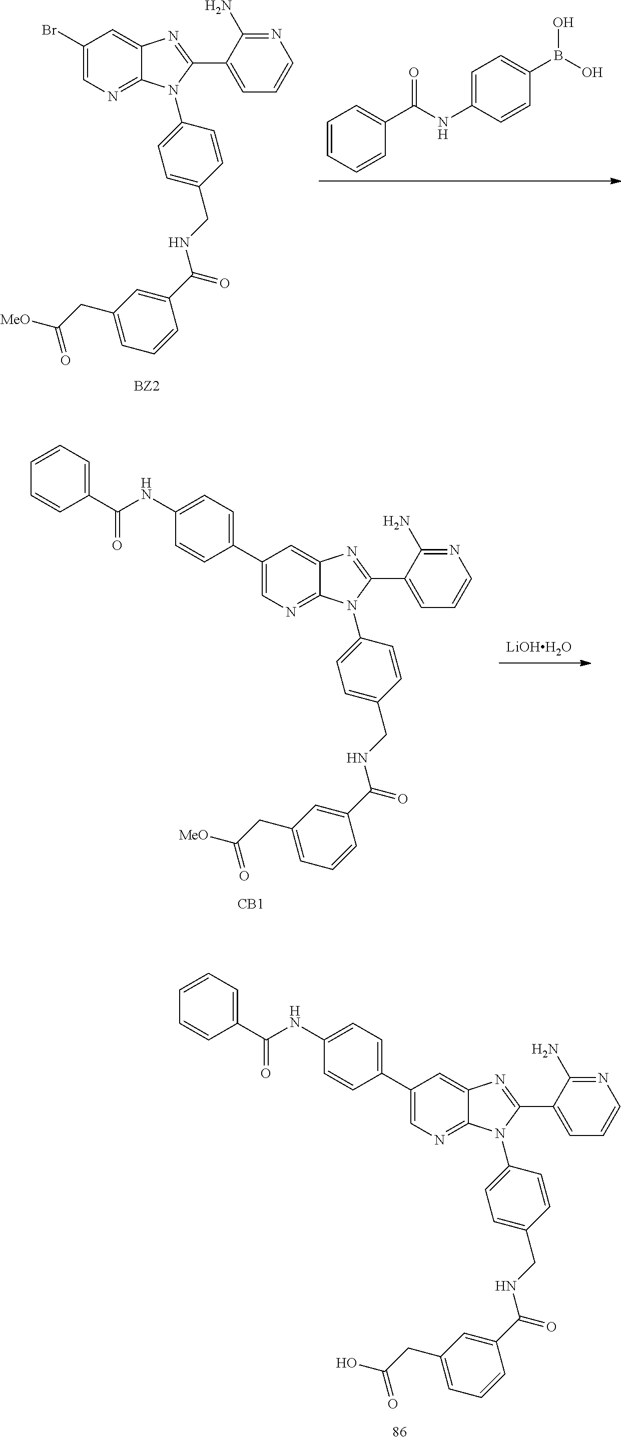

Step 1. To a solution of (4-benzamidophenyl) boronic acid (190 mg, 0.780 mmol) and methyl 2-(3-((4-(2-(2-aminopyridin-3-yl)-6-bromo-3H-imidazo[4,5-b]pyridin-3-yl)benzyl)carbamoyl)phenyl)acetate BZ2 (150 mg, 0.260 mmol) in 1,4-dioxane (5.00 mL) and water (1.50 mL), K₂CO₃ (109 mg, 0.780 mmol) and Pd(dppf)Cl₂ (19.0 mg, 0.0200 mmol) were added, the reaction mixture was degassed and purged with N₂ (×3), and the mixture was stirred at 90° C. for 12 hrs. The reaction mixture was filtered, the filtrate was concentrated, and the resulting residue was purified by combi-flash column (EtOAc in PE, 100%) to give methyl 2-(3-((4-(2-(2-aminopyridin-3-yl)-6-(4-benzamidophenyl)-3H-imidazo[4,5-b]pyridin-3-yl)benzyl)carbamoyl)phenyl)acetate CB1 (150 mg, 71.4% yield) as a yellow solid. ¹H NMR (400 MHz, DMSO-d₆) δ_{H}=10.38 (s, 1H), 9.14 (t, J=2.0 Hz, 1H), 8.63 (d, J=2.0 Hz, 1H), 8.46 (d, J=2.0 Hz, 1H), 8.01-7.98 (m, 2H), 7.97-7.91 (m, 4H), 7.82-7.75 (m, 4H), 7.60~ 7.52 (m, 4H), 7.47-7.43 (m, 4H), 7.27 (dd, J=2.0, 7.6 Hz, 1H), 7.03 (s, 2H), 6.43 (dd, J=4.8, 7.6 Hz, 1H), 4.58 (d, J=5.6 Hz, 2H), 3.77 (s, 2H), 3.63 (s, 3H).

Step 2. To a solution of methyl 2-(3-((4-(2-(2-aminopyridin-3-yl)-6-(4-benzamidophenyl)-3H-imidazo[4,5-b]pyridin-3-yl)benzyl)carbamoyl)phenyl)acetate CB1 (150 mg, 0.220 mmol) in THF (2.00 mL) and water (1.00 mL), LiOH·H₂O (37.0 mg, 0.870 mmol) was added and the mixture was stirred at 25° C. for 2 hrs. The reaction mixture was diluted with DMF (4 mL) and purified by prep-HPLC (Column: Welch Xtimate C18 150*40 mm*5 μm; mobile phase: [water (HCl)-ACN]; B %: 8%-48%; 36 min) to give 2-(3-((4-(2-(2-aminopyridin-3-yl)-6-(4-benzamidophenyl)-3H-imidazo[4,5-b]pyridin-3-yl)benzyl)carbamoyl)phenyl) acetic acid 86 (19.8 mg, 13.4% yield) as a yellow solid. ¹H NMR (400 MHz, DMSO-d₆) δ_{H}=10.42 (s, 1H), 9.16 (1, J=2.0 Hz, 1H), 8.74 (d, J=2.0 Hz, 1H), 8.56-8.38 (d, J==2.0 Hz, 2H), 8.11 (dd, J=1.6, 6.4 Hz, 1H), 8.03-7.90 (m, 5H), 7.86-7.78 (m, 4H), 7.65-7.59 (m, 1H), 7.58-7.53 (m, 2H), 7.53-7.47 (m, 4H), 7.46-7.40 (m, 2H), 6.93-6.87 (m, 1H), 4.58 (d, J=5.6 Hz, 2H), 3.65 (s, 2H). HPLC R; =2.870 min in 8 min chromatography, purity 99.9%. LCMS Rₜ=1.716 min in 4 min; Agilent PoroShell 120 EC-C18 2.7 μm 3.0*50 mm; purity 100%, MS ESI calcd, for 673.24, [M+H]⁺ 674.24, found 674.2.

### Example 87. Synthesis of methyl 2-(4-((4-(2-(2-aminopyridin-3-yl)-6-(pyridin-3-yl)-3H-imidazo[4,5-b]pyridin-3-yl)benzyl)carbamoyl)phenyl)acetate (Compound 87)

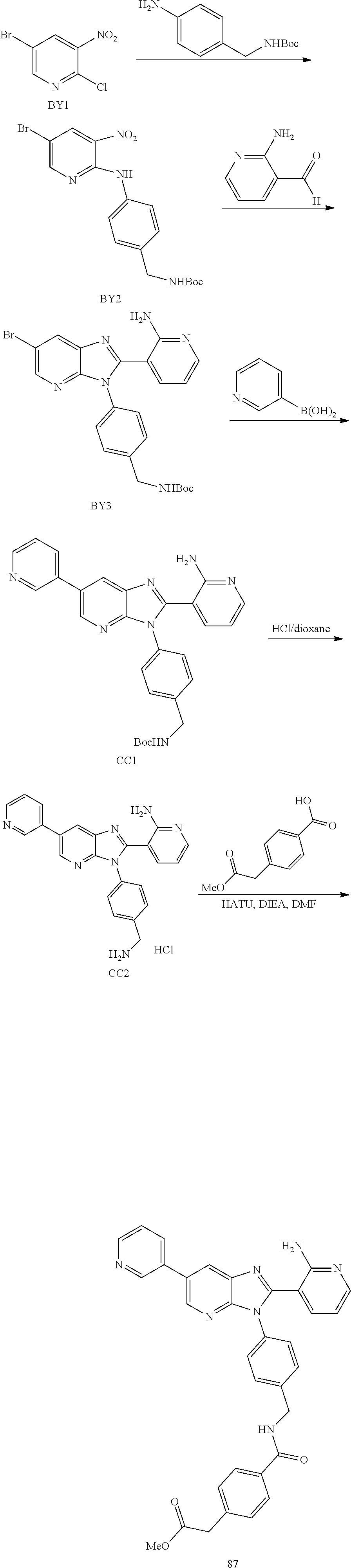

Step 1. To a solution of 5-bromo-2-chloro-3-nitro-pyridine BY1 (1.00 g, 4.21 mmol) in 1,4-dioxane (20.0 mL), tert-butyl N-[(4-aminophenyl)methyl]carbamate (0.940 g, 4.21 mmol) and DIEA (1.10 mL, 6.32 mmol) were added at 25° C., and the mixture was stirred at 80° C. for 16 hrs. The reaction mixture was concentrated directly and the resulting residue was purified by flash silica gel chromatography (eluent with 0-15% of EtOAc in PE) to give tert-butyl N-[[4-[(5-bromo-3-nitro-2-pyridyl)amino]phenyl]methyl]carbamate BY2 (1.20 g, 2.84 mmol, 67.3% yield) as a red solid. ¹H NMR (400 MHz, DMSO-d₆) δ=9.93 (s, 1H), 8.67 (d, J=2.0 Hz, 1H), 8.58 (d, J=2.4 Hz, 1H), 7.50 (d, J=8.0 Hz, 2H), 7.40 (t, J=6.0 Hz, 1H), 7.22 (d, J=8.4 Hz, 2H), 4.11 (d, J=6.0 Hz, 2H), 1.39 (s, 9H).

Step 2. To a solution of tert-butyl N-[4-[(5-bromo-3-nitro-2-pyridyl)amino]phenyl]methyl]carbamate BY2 (1.20 g, 2.84 mmol) in DMSO (12.0 mL) and methanol (12.0 mL), 2-aminopyridine-3-carbaldehyde (467 mg, 3.83 mmol) and Na₂S₂O₄ (1.23 g. 7.09 mmol) were added at 25° C., and the mixture was stirred at 100° C. for 16 hrs. The reaction mixture was concentrated, water (10 mL) was added, and the resulting mixture was extracted with EtOAc (10 mL×3). The combined organic phase was washed with brine (10 mL), water (10 mL), dried over anhydrous Na₂SO₄, filtered and concentrated to dryness. The residue was purified by flash silica gel chromatography (eluent with 0-44% of EtOAc in PE) to give tert-butyl N-[[4-[2-(2-amino-3-pyridyl)-6-bromo-imidazo[4,5-b]pyridin-3-yl]phenyl methyl]carbamate BY3 (666 mg, 1.34 mmol, 47.4% yield) as a yellow oil. ¹H NMR (400 MHz, CHLOROFORM-d) δ=8.91 (d, J=2.0 Hz, 1H), 8.66 (dd, J=4.8, 1.2 Hz, 1H), 8.60 (d, J=2.0 Hz, 1H), 8.28 (d, J=2.0 Hz, 1H), 8.04 (dd . . . ]=5.2, 1.6 Hz, 1H), 7.94 (dt, J=7.6, 1.6 Hz, 1H), 7.50 (d, J=8.4 Hz, 2H), 7.47-7.42 (m, 1H), 7.39 (d, J=8.4 Hz, 2H), 4.99 (s, 1H), 4.44 (d, J=5.6 Hz, 2H), 1.48 (s, 9H).

Step 3. Tert-butyl N-[4-[2-(2-amino-3-pyridyl)-6-bromo-imidazo[4,5-b]pyridin-3-yl]phenyl]methyl]carbamate BY3 (330 mg, 0.666 mmol) and 3-pyridylboronic acid (246 mg, 2.00 mmol) were suspended in a mixture of ethanol (5.00 mL) and toluene (5.00 mL). A solution of aqueous NaHCO₃ (1.00 mL, 1.1 M) was added and the mixture was degassed with nitrogen for 5 mins. Pd(PPh₃)₄ (38.0 mg, 0.0333 mmol) was added and the mixture was heated to 100° C. for 16 hrs. The reaction mixture was concentrated to dryness and the resulting residue was purified by flash silica gel chromatography (eluent with 0-64% of EtOAc/PE) to give tert-butyl N-[4-[2-(2-amino-3-pyridyl)-6-(3-pyridyl) imidazo[4,5-b]pyridin-3-yl]phenyl methyl]carbamate CC1 (101 mg, 0.205 mmol, 30.7% yield) as a brown oil. ¹H NMR (400 MHz, DMSO-d₆) δ=9.01 (d, J=2.0 Hz, 1H), 8.67 (d, J=2.0 Hz, 1H), 8.62 (dd, J=1.6, 4.8 Hz, 1H), 8.57 (d, J=2.0 Hz, 1H), 8.24-8.18 (m, 1H), 8.01 (dd, J=1.6, 4.8 Hz, 1H), 7.55 (dd, J=5.2, 7.8 Hz, 1H), 7.49 (1, J=4.8 Hz, 1H), 7.45-7.37 (m, 4H), 7.25 (dd, J=1.6, 7.6 Hz, 1H), 7.03 (s, 2H), 6.42 (dd, J=4.4, 7.2 Hz, 1H), 4.22 (d, J=6.0 Hz, 2H), 1.41 (s, 9H).

Step 4. To a solution of tert-butyl N-[[4-[2-(2-amino-3-pyridyl)-6-(3-pyridyl) imidazo[4,5-b]pyridin-3-yl]phenyl]methyl]carbamate CC1 (100 mg, 0.203 mmol) in methanol (2.00 mL). HCl/dioxane (2.00 mL, 4 M) was added at 25° C., and the mixture was stirred for 16 hrs. The reaction mixture was concentrated directly, and the resultant residue was purified by prep-HPLC (Column: Phenomenex C18 75*30 mm*3 μm; Condition: water (HCl)-ACN; Begin B: 0%-24%) to give 3-(3-(4-(aminomethyl)phenyl)-6-(pyridin-3-yl)-3H-imidazo[4,5-b]pyridin-2-yl)pyridin-2-amine hydrochloride CC2 (35.0 mg, 0.0886 mmol, 43.7% yield) as a yellow oil. JH NMR (400 MHz, DMSO-d₆) δ=9.27 (s, 1H), 8.87 (d, J=2.0 Hz, 1H), 8.83 (d, J=5.2 Hz, 1H), 8.79 (d, J=2.0 Hz, 1H), 8.70 (d, J=6.4 Hz, 1H), 8.62-8.24 (m, 5H), 8.16 (dd, J=1.6, 6.0 Hz, 1H), 7.95 (d, J=7.6 Hz, 2H), 7.72-7.66 (m, 2H), 7.64-7.58 (m, 2H), 6.90 (t, J=6.8 Hz, 1H), 4.12 (d, J=6.0 Hz, 2H). HPLC Rₜ=1.354 min in 8 min chromatography, purity 99.3%. LCMS Rₜ=0.959 min in 2 min chromatography, purity 98.6%, MS ESI calcd, for 393.17 [M+H]⁺ 394.17, found 394.0.

Step 5. To a solution of 4-(2-methoxy-2-oxo-ethyl)benzoic acid (99.0 mg, 0.508 mmol) in DMF (2.00 mL), 3-[3-[4-(aminomethyl)phenyl]-6-(3-pyridyl) imidazo[4,5-b]pyridin-2-yl]pyridin-2-amine CC2 (100 mg, 0.254 mmol), DIEA (0.130 mL, 0.762 mmol) and HATU (145 mg, 0.381 mmol) were added at 25° C. and the mixture was stirred for 16 hrs. The residue was purified by prep-HPLC (Column: Welch Xtimate C18 150*30 mm*5 μm; Condition: water (FA)-ACN; Begin B: 0-34%) to give methyl 2-(4-((4-(2-(2-aminopyridin-3-yl)-6-(pyridin-3-yl)-3H-imidazo[4,5-b]pyridin-3-yl)benzyl)carbamoyl)phenyl)acetate 87 (34.0 mg, 0.0597 mmol, 23.5% yield) as a yellow solid. ¹H NMR (400 MHz, DMSO-d₆) δ=9.13 (t, J=6.0 Hz, 1H), 9.01 (d, J=2.0 Hz, 1H), 8.67 (d, J=2.0 Hz, 1H), 8.62 (dd, J=4.4, 1.2 Hz, 1H), 8.58 (d, J=2.0 Hz, 1H), 8.21 (dt, J=8.0, 1.6 Hz, 1H), 8.01 (dd, J=4.8, 4.4 Hz, 1H), 7.88 (d, J=8.4 Hz, 2H), 7.55 (dd, J=8.4, 4.8 Hz, 1H), 7.45 (q, J=8.4 Hz, 4H), 7.38 (d, J=8.4 Hz, 2H), 7.29 (dd, J=7.2, 2.0 Hz, 1H), 7.05 (s, 2H), 6.44 (dd, J=7.6, 4.8 Hz, 1H), 4.58 (d, J=6.0 Hz, 2H), 3.77 (s, 2H), 3.62 (s, 3H). HPLC Rₜ=3.980 min in 8 min chromatography, purity 99.3%. LCMS Rₜ=0.972 min in 2.5 min chromatography, purity 98.4%, MS ESI calcd. for 569.22 [M+H]⁺ 570.22, found 570.3.

### Example 88. Synthesis of 2-(4-((4-(2-(2-aminopyridin-3-yl)-6-(pyridin-3-yl)-3H-imidazo[4,5-b]pyridin-3-yl)benzyl)carbamoyl)phenyl) acetic acid (Compound 88)

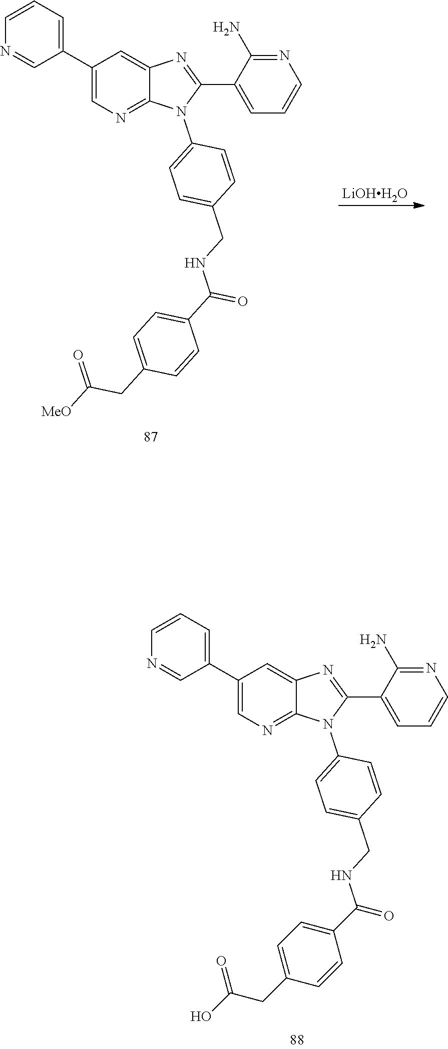 [SMILES: COC(=O)Cc1ccc(C(=O)NCc2ccc(-n3c(-c4cccnc4N)nc4cc(-c5cccnc5)cnc43)cc2)cc1 ; "LiOH·H2O" ; Nc1ncccc1-c1nc2cc(-c3cccnc3)cnc2n1-c1ccc(CNC(=O)c2ccc(CC(=O)O)cc2)cc1]

Step 1. To a solution of methyl 2-(4-((4-(2-(2-aminopyridin-3-yl)-6-(pyridin-3-yl)-3H-imidazo[4,5-b]pyridin-3-yl)benzyl)carbamoyl)phenyl)acetate 87 (90.0 mg, 0.158 mmol) in THF (2.00 mL) and water (2.00 mL), LiOH·H₂O (20.0 mg, 0.474 mmol) was added at 25° C., and the mixture was stirred for 2 hrs. The residue was purified by prep-HPLC (Column: Welch Xtimate C18 150*30 mm*5 μm; Condition: water (FA)-ACN; Begin B: 0-34%) to give 2-(4-((4-(2-(2-aminopyridin-3-yl)-6-(pyridin-3-yl)-3H-imidazo[4,5-b]pyridin-3-yl)benzyl)carbamoyl)phenyl) acetic acid 88 (42.0 mg, 0.0761 mmol, 48.2% yield) as a white solid. ¹H NMR (400 MHz, DMSO-d₆) δ=9.12 (t, J=6.0 Hz, 1H), 9.01 (d, J=2.0 Hz, 1H), 8.66 (d, J=2.0 Hz, 1H), 8.62 (dd, J=4.4, 1.2 Hz, 1H), 8.58 (d, J=2.0 Hz, 1H), 8.21 (dt, J=8.0, 1.2 Hz, 1H), 8.14 (s, 1H), 8.01 (dd, J=4.8, 1.6 Hz, 1H), 7.87 (d, J=8.4 Hz, 2H), 7.54 (dd, J=8.0, 4.8 Hz, 1H), 7.50-7.41 (m, 4H), 7.37 (d, J=8.4 Hz, 2H), 7.28 (dd, J=7.6, 2.0 Hz, 1H), 7.03 (s, 2H), 6.43 (dd, J=7.6, 4.8 Hz, 1H), 4.58 (d, J=6.0 Hz, 2H), 3.65 (s, 2H). HPLC Rₜ=3.499 min in 8 min chromatography, purity 97.6%. LCMS Rₜ=0.886 min in 2.5 min chromatography, purity 97.5%, MS ESI calcd, for 555.20 [M+H]⁺ 556.20, found 556.2.

### Example 89. Synthesis of methyl 2-(3-((4-(2-(2-aminopyridin-3-yl)-6-(pyridin-3-yl)-3H-imidazo[4,5-b]pyridin-3-yl)benzyl)carbamoyl)phenyl)acetate (Compound 89)

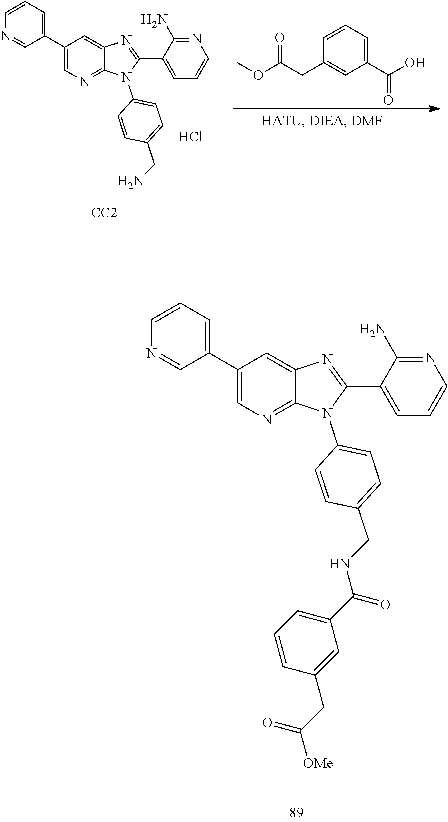

Step 1. To a solution of 3-(2-methoxy-2-oxo-ethyl)benzoic acid (99.0 mg, 0.508 mmol) in DMF (2.00 mL) 3-(3-(4-(aminomethyl)phenyl)-6-(pyridin-3-yl)-3H-imidazo[4,5-b]pyridin-2-yl)pyridin-2-amine hydrochloride CC2 (100 mg, 0.254 mmol), DIEA (0.130 mL, 0.762 mmol) and HATU (145 mg, 0.381 mmol) were added at 25° C. and the mixture was stirred for 16 hrs. The residue was purified by prep-HPLC (Column: Welch Xtimate C18 150*30 mm*5 μm; Condition: water (FA)-ACN; Begin B: 0-34%) to give methyl 2-(3-((4-(2-(2-aminopyridin-3-yl)-6-(pyridin-3-yl)-3H-imidazo[4,5-b]pyridin-3-yl)benzyl)carbamoyl)phenyl)acetate 89 (30.0 mg, 0.0518 mmol, 20.4% yield) as a yellow solid. ¹H NMR (400 MHz, DMSO-d₆) δ=9.15 (t, J=5.6 Hz, 1H), 9.01 (d, J=2.0 Hz, 1H), 8.67 (d, J=2.0 Hz, 1H), 8.62 (dd, J=1.6, 4.8 Hz, 1H), 8.58 (d, J=2.0 Hz, 1H), 8.21 (dt, J=8.0, 2.0 Hz, 1H), 8.01 (dd, J=4.8, 2.0 Hz, 1H), 7.85-7.79 (m, 2H), 7.55 (dd, J=8.0, 4.8 Hz, 1H), 7.50-7.40 (m, 6H), 7.30 (dd, J=7.6, 2.0 Hz, 1H), 7.06 (s, 2H), 6.44 (dd, J=8.0, 5.2 Hz, 1H), 4.58 (d, J=6.0 Hz, 2H), 3.77 (s, 2H), 3.62 (s, 3H). HPLC Rₜ=4.069 min in 8 min chromatography, purity 99.0%. LCMS Rₜ=0.971 min in 2.5 min chromatography, purity 99.0%, MS ESI calcd. For 569.22 [M+H]⁺ 570.22, found 570.3.

### Example 90. Synthesis of 2-(3-((4-(2-(2-aminopyridin-3-yl)-6-(pyridin-3-yl)-3H-imidazo[4,5-b]pyridin-3-yl)benzyl)carbamoyl)phenyl) acetic acid (Compound 90)

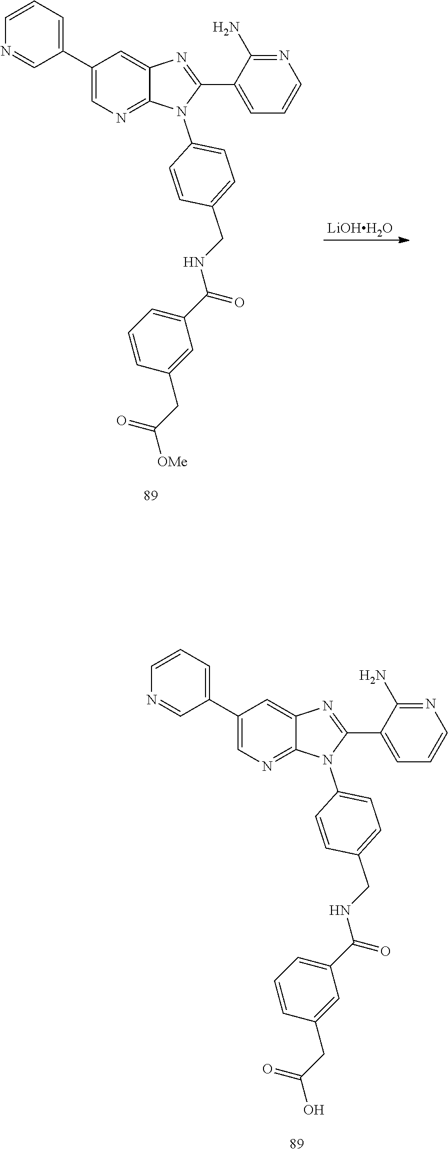 [SMILES: COC(=O)Cc1cccc(C(=O)NCc2ccc(-n3c(-c4cccnc4N)nc4cc(-c5cccnc5)cnc43)cc2)c1 ; "LiOH·H2O" ; Nc1ncccc1-c1nc2cc(-c3cccnc3)cnc2n1-c1ccc(CNC(=O)c2cccc(CC(=O)O)c2)cc1]

Step 1. To a solution of methyl 2-(3-((4-(2-(2-aminopyridin-3-yl)-6-(pyridin-3-yl)-3H-imidazo[4,5-b]pyridin-3-yl)benzyl)carbamoyl)phenyl)acetate 89 (100 mg, 0.176 mmol) in THF (2.00 mL) and water (2.00 mL), LiOH·H₂O) (22.0 mg, 0.527 mmol) was added at 25° C. and the mixture was stirred for 2 hrs. The reaction mixture was concentrated and the residue was purified by prep-HPLC (Column: Welch Xtimate C18 150*30 mm*5 μm; Condition: water (FA)-ACN; Begin B: 0-30%) to give 2-(3-((4-(2-(2-aminopyridin-3-yl)-6-(pyridin-3-yl)-3H-imidazo[4,5-b]pyridin-3-yl)benzyl)carbamoyl)phenyl) acetic acid 90 (44.0 mg, 0.0785 mmol, 44.7% yield) as a white solid. ¹H NMR (400 MHz, DMSO-d₆) δ=9.14 (t, J=6.0 Hz, 1H), 9.01 (d, J=1.6 Hz, 1H), 8.67 (d, J=2.0 Hz, 1H), 8.62 (dd, J=4.8, 1.6 Hz, 1H), 8.58 (d, J=2.0 Hz, 1H), 8.21 (dt, J=8.0, 1.6 Hz, 1H), 8.01 (dd, J=4.8, 2.0 Hz, 1H), 7.84-7.78 (m, 2H), 7.54 (dd, J=8.0, 4.8 Hz, 1H), 7.50-7.41 (m, 6H), 7.28 (dd, J=7.6, 1.6 Hz, 1H), 7.03 (s, 2H), 6.43 (dd, J=7.6, 4.8 Hz, 1H), 4.58 (d, J=6.0 Hz, 2H), 3.65 (s, 2H). HPLC Rₜ=3.550 min in 8 min chromatography, purity 99.6%. LCMS Rₜ=0.901 min in 2.5 min chromatography, purity 99.8%, MS ESI calcd, for 555.20 [M+H]⁺ 556.20, found 556.2.

### Example 91. Synthesis of methyl 3-(4-((4-(2-(2-aminopyridin-3-yl)-6-(pyridin-3-yl)-3H-imidazo[4,5-b]pyridin-3-yl)benzyl)carbamoyl)phenyl) propanoate (Compound 91)

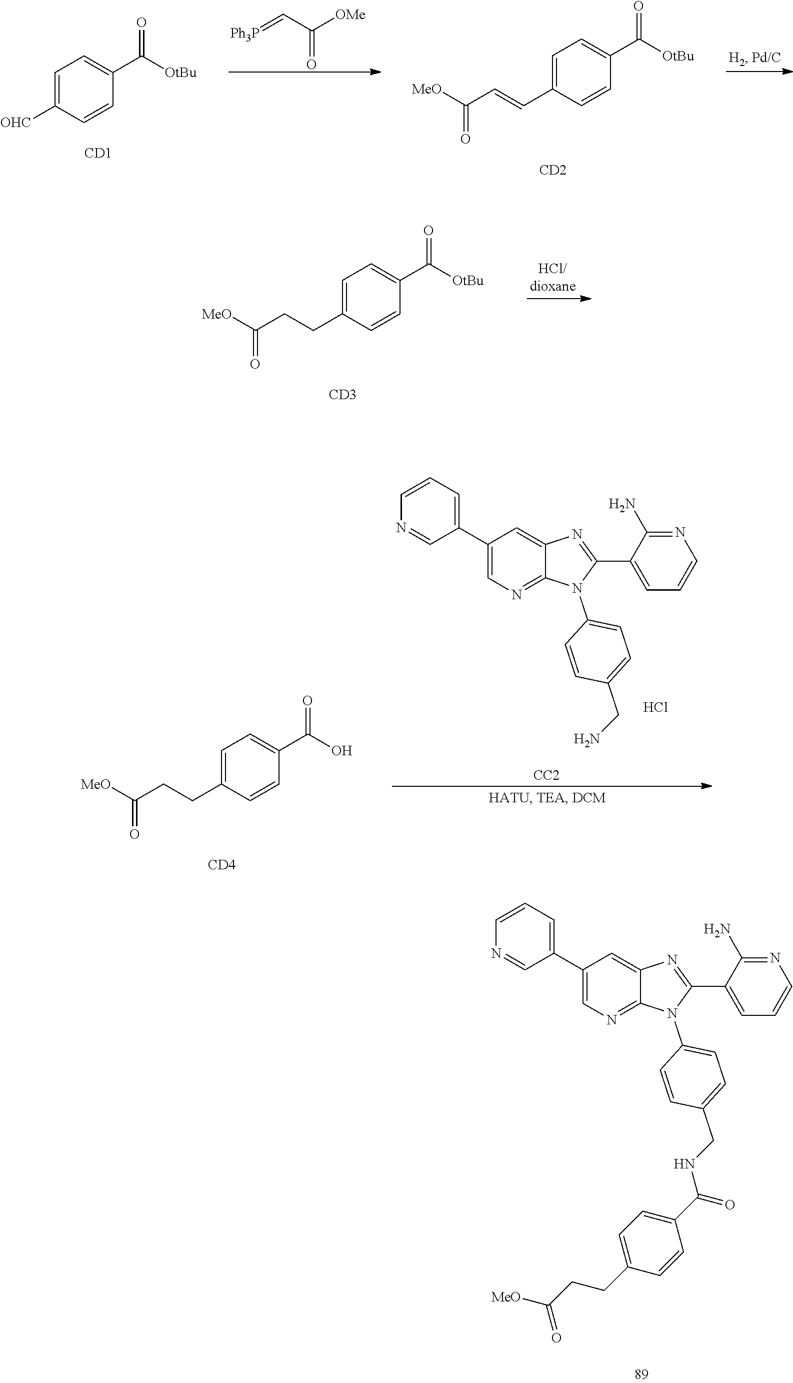

Step 1. To a solution of tert-butyl 4-formylbenzoate CD1 (2.00 g, 9.70 mmol) in toluene (5.00 mL), methyl 2-(triphenyl-λ5-phosphanylidene)acetate (6.40 g, 19.4 mmol) was added at 25° C., and the mixture was stirred at 110° C. for 2 hrs. The reaction mixture was filtered, the filter cake was washed with toluene (10 mL) and then dried. The reaction mixture was concentrated directly. The residue was purified by flash silica gel chromatography (eluent with 0-10% of EtOAc in PE) to give tert-butyl 4-[(E)-3-methoxy-3-oxo-prop-1-enyl]benzoate CD2 (2.10 g. 8.01 mmol, 82.6% yield) as a white solid. ¹H NMR (400 MHz, DMSO-d₆) δ=7.96-7.81 (m, 4H), 7.71 (d, J=16.0 Hz, 1H), 6.76 (d, J=16.0 Hz, 1H), 3.74 (s, 3H), 1.55 (s, 9H).

Step 2. To a solution of tert-butyl 4-[(E)-3-methoxy-3-oxo-prop-1-enyl]benzoate CD2 (250 mg, 0.953 mmol) in methanol (5.00 mL), Pd/C (wet, 10%, 0.200 g) was added under N₂ atmosphere. The suspension was degassed and purged with H₂ (×3), and the reaction mixture was stirred under H₂ (15 psi) at 25° C. for 16 hrs. The reaction mixture was filtered and the filter cake was washed with MeOH (20 mL), dried over Na₂SO₄, filtered and concentrated to give tert-butyl 4-(3-methoxy-3-oxo-propyl)benzoate CD3 (220 mg, 0.832 mmol, 87.3% yield) as a white solid. ¹H NMR (400 MHz, DMSO-d₆) δ=7.84-7.76 (m, 2H), 7.37-7.28 (m, 2H), 3.57 (s, 3H), 2.95-2.86 (m, 2H), 2.70-2.61 (m, 2H), 1.53 (s, 9H).

Step 3. To a solution of tert-butyl 4-(3-methoxy-3-oxo-propyl)benzoate CD3 (200 mg, 0.757 mmol) in 1,4-dioxane (1.00 mL), HCl/dioxane (1.00 mL) was added at 25° C., and the mixture was stirred for 2 hrs. The reaction mixture was concentrated directly to give 4-(3-methoxy-3-oxo-propyl)benzoic acid CD4 (150 mg, 0.720 mmol, 95.2% yield) as a white solid.

Step 4. To a solution of 3-(3-(4-(aminomethyl)phenyl)-6-(pyridin-3-yl)-3H-imidazo [4,5-b]pyridin-2-yl)pyridin-2-amine hydrochloride CC2 (200 mg, 0.508 mmol) in DCM (6.00 mL), 4-(3-methoxy-3-oxo-propyl)benzoic acid CD4 (212 mg, 1.02 mmol), TEA (0.210 mL, 1.52 mmol) and HATU (290 mg, 0.762 mmol) were added at 25° C., and the mixture was stirred for 16 hrs. The reaction mixture was concentrated directly and the residue was purified by flash silica gel chromatography (eluent of 0-20% MeOH/DCM (@ 45 mL/min) and prep-HPLC (Column: Welch Xtimate C18 150*30 mm*5 μm; Condition: water (FA)-ACN; Begin B: 0-36%) to give methyl 3-(4-((4-(2-(2-aminopyridin-3-yl)-6-(pyridin-3-yl)-3H-imidazo[4,5-b]pyridin-3-yl)benzyl)carbamoyl)phenyl) propanoate 91 (28.0 mg, 0.0476 mmol, 9.37% yield) as a white solid. ¹H NMR (400 MHz, DMSO-d₆) δ=9.08 (t, J=6.0 Hz, 1H), 9.01 (d, J=2.0 Hz, 1H), 8.66 (d, J]=2.0 Hz, 1H), 8.62 (dd, J=4.8, 1.6 Hz, 1H), 8.57 (d, J=2.0 Hz, 1H), 8.20 (dt, J=8.0, 2.0 Hz, 1H), 8.01 (dd, J=4.8, 1.6 Hz, 1H), 7.84 (d, J=8.4 Hz, 2H), 7.54 (dd, J=8.0, 4.8 Hz, 1H), 7.45 (q, J=8.8 Hz, 4H), 7.34 (d, J=8.0 Hz, 2H), 7.27 (dd, J=7.6, 1.6 Hz, 1H), 7.03 (s, 2H), 6.43 (dd, J=7.6, 4.8 Hz, 1H), 4.57 (d, J=6.0 Hz, 2H), 3.58 (s, 3H), 2.95-2.87 (m, 2H), 2.70-2.64 (m, 2H). HPLC Rₜ=4.283 min in 8 min chromatography, purity 99.4%. LCMS Rₜ=0.888 min in 2 min chromatography, purity 99.0%, MS ESI calcd, for 583.23 [M+H]⁺ 584.23, found 584.1.

### Example 92. Synthesis of 3-(4-((4-(2-(2-aminopyridin-3-yl)-6-(pyridin-3-yl)-3H-imidazo[4,5-b]pyridin-3-yl)benzyl)carbamoyl)phenyl) propanoic acid (Compound 92)

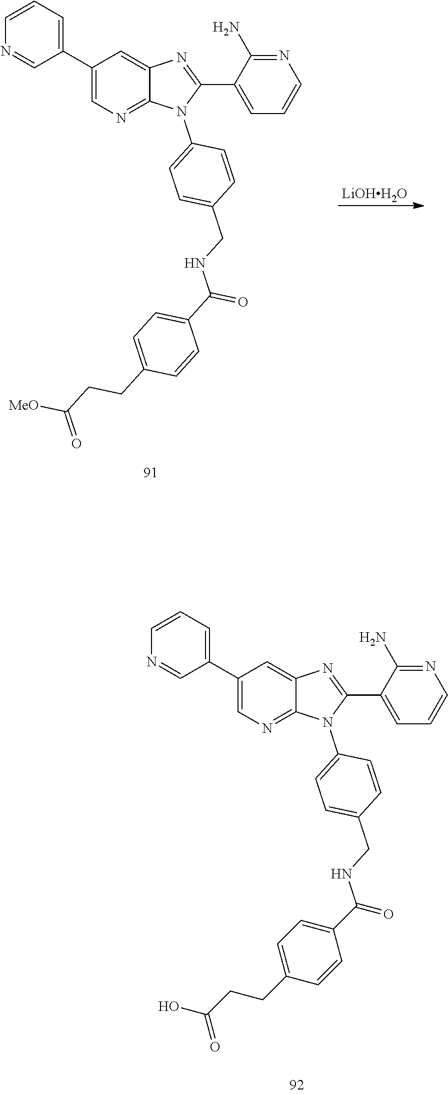 [SMILES: COC(=O)CCc1ccc(C(=O)NCc2ccc(-n3c(-c4cccnc4N)nc4cc(-c5cccnc5)cnc43)cc2)cc1 ; "LiOH·H2O" ; Nc1ncccc1-c1nc2cc(-c3cccnc3)cnc2n1-c1ccc(CNC(=O)c2ccc(CCC(=O)O)cc2)cc1]

Step 1. To a solution of methyl 3-(4-((4-(2-(2-aminopyridin-3-yl)-6-(pyridin-3-yl)-3H-imidazo[4,5-b]pyridin-3-yl)benzyl)carbamoyl)phenyl) propanoate 91 (200 mg, 0.343 mmol) in THF (2.00 mL) and water (2.00 mL), LiOH·H₂O (43.0 mg, 1.03 mmol) was added at 25° C., and the mixture was stirred for 16 hrs. The reaction mixture was concentrated to remove THF and the residue was purified by prep-HPLC (Column: Welch Xtimate C18 150*30 mm*5 μm; Condition: water (FA)-ACN: Begin B: 0-32%) to give 3-(4-((4-(2-(2-aminopyridin-3-yl)-6-(pyridin-3-yl)-3H-imidazo[4,5-b]pyridin-3-yl)benzyl)carbamoyl)phenyl) propanoic acid 92 (113 mg, 0.255 mmol, 34.3% yield) as a white solid. ¹H NMR (400 MHz, DMSO-d₆) δ=9.08 (t, J=6.0 Hz, 1H), 9.01 (d, J=2.0 Hz, 1H), 8.66 (d, J=2.0 Hz, 1H), 8.62 (dd, J=4.4, 1.2 Hz, 1H), 8.57 (d, J=2.0 Hz, 1H), 8.20 (dt, J=8.0, 1.6 Hz, 1H), 8.01 (dd, J=4.8, 2.0 Hz, 1H), 7.84 (d, J=8.0 Hz, 2H), 7.54 (dd, J=8.0, 4.4 Hz, 1H), 7.45 (q, J=8.4 Hz, 4H), 7.34 (d, J=8.0 Hz, 2H), 7.27 (dd, J=7.6, 1.6 Hz, 1H), 7.03 (s, 2H), 6.43 (dd, J=7.6, 4.8 Hz, 1H), 4.57 (d, J=6.0 Hz, 2H), 2.87 (t, J=7.6 Hz, 2H), 2.57 (t, J=7.2 Hz, 2H). HPLC Rₜ=3.726 min in 8 min chromatography, purity 99.6%. LCMS Rₜ=0.760 min in 2 min chromatography, purity 99.4%, MS ESI calcd, for 569.22 [M+H]⁺ 570.22, found 570.0.

### Example 93. Synthesis of methyl 3-(3-((4-(2-(2-aminopyridin-3-yl)-6-(pyridin-3-yl)-3H-imidazo[4,5-b]pyridin-3-yl)benzyl)carbamoyl)phenyl) propanoate (Compound 93)

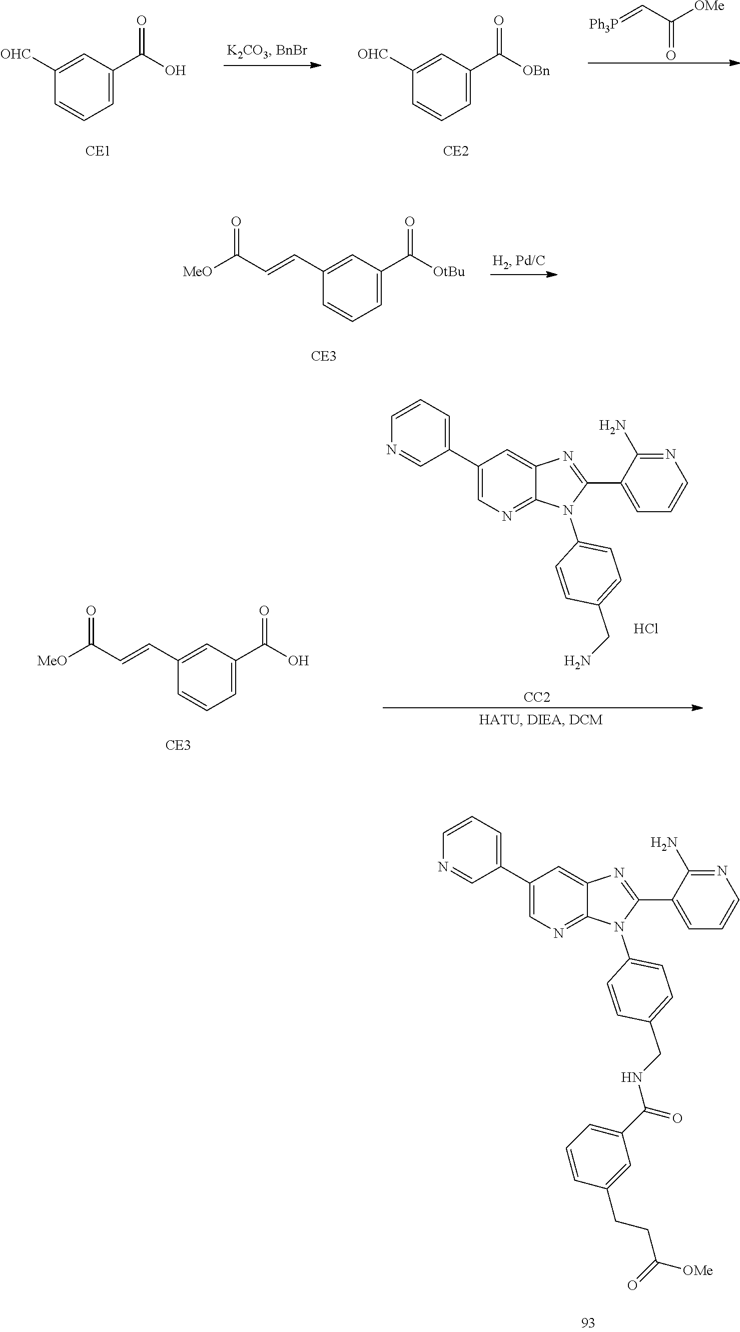

Step 1. To a solution of 3-formylbenzoic acid CE1 (1.00 g, 6.66 mmol) in MeCN (10.0 mL), bromomethylbenzene (0,950 mL, 7.99 mmol) and K₂CO₃ (2.70 g, 20.0 mmol) were added at 25° C. and the mixture was stirred for 2 hrs. The reaction mixture was concentrated directly and the resulting residue was purified by flash silica gel chromatography (eluent with 0-20% of EtOAc/PE @ 45 mL/min) to give benzyl 3-formylbenzoate CE2 (1.50 g, 6.24 mmol, 93.7% yield) as a colorless oil. ¹H NMR (400 MHz, DMSO-d₆) δ=10.11 (s, 1H), 8.48 (s, 1H), 8.30 (d, J=7.6 Hz, 1H), 8.19 (d, J=7.6 Hz, 1H), 7.78 (t, J=7.6 Hz, 1H), 7.53-7.48 (m, 2H), 7.46-7.35 (m, 3H), 5.41 (s, 2H).

Step 2. To a solution of benzyl 3-formylbenzoate CE2 (2.16 g, 8.97 mmol) in toluene (20.0 mL), methyl 2-(triphenyl-λ5-phosphanylidene)acetate (2.00 g, 5.98 mmol) was added at 25° C., and the mixture was stirred at 110° C. for 3 hrs. The reaction mixture was concentrated directly and the resulting residue was purified by flash silica gel chromatography (eluent of 0-20% EtOAc/PE @ 45 mL/min) to give benzyl 3-[(E)-3-methoxy-3-oxo-prop-1-enyl]benzoate CE3 (1.50 g, 5.06 mmol, 84.6% yield) as a colorless oil. ¹H NMR (400 MHz, DMSO-d₆) δ=8.25 (s, 1H), 8.04 (dd, J=14.4, 7.6 Hz, 2H), 7.76 (d, J=16.0 Hz, 1H), 7.59 (t, J=8.0 Hz, 1H), 7.53-7.46 (m, 2H), 7.45-7.32 (m, 3H), 6.74 (d, J=16.0 Hz, 1H), 5.38 (s, 2H), 3.73 (s, 3H).

Step 3. To a solution of benzyl 3-[(E)-3-methoxy-3-oxo-prop-1-enyl]benzoate CE3 (2.00 g, 6.75 mmol) in methanol (20.0 mL), Pd/C (wet, 10%, 1.10 g) was added under N₂ atmosphere. The suspension was degassed and purged with H₂ (×3), and the reaction mixture was stirred under H₂ (30 Psi) at 25° C. for 16 hrs. The reaction mixture was filtered, the filter cake was washed with MeOH (100 mL) and then dried providing 3-(3-methoxy-3-oxo-propyl)benzoic acid CE4 (1.10 g, 5.28 mmol, 78.3% yield) as a white solid. JH NMR (400 MHz, DMSO-d₆) δ=7.81-7.72 (m, 2H), 7.48-7.35 (m, 2H), 3.57 (s, 3H), 2.94-2.87 (m, 2H), 2.68-2.62 (m, 2H).

Step 4 To a solution of 3-(3-(4-(aminomethyl)phenyl)-6-(pyridin-3-yl)-3H-imidazo[4,5-b]pyridin-2-yl)pyridin-2-amine hydrochloride CC2 (300 mg, 0.762 mmol) in DCM (6.00 mL), 3-(3-methoxy-3-oxo-propyl)benzoic acid CE4 (238 mg, 1.14 mmol) DIEA (0.320 mL, 2.29 mmol) and HATU (435 mg, 1.14 mmol) were added at 25° C., and the mixture was stirred for 16 hrs. The reaction mixture was concentrated directly and the resulting residue was purified by flash silica gel chromatography (eluent of 0-11% MeOH/DCM @, 45 mL/min) and prep-HPLC (Column: Welch Xtimate C18 150*30 mm*5 μm; Condition: water (FA)-ACN; Begin B: 0-32%) to give methyl 3-(3-((4-(2-(2-aminopyridin-3-yl)-6-(pyridin-3-yl)-3H-imidazo[4,5-b]pyridin-3-yl)benzyl)carbamoyl)phenyl) propanoate 93 (32.0 mg, 0.0536 mmol, 7.03% yield) as a white solid. ¹H NMR (400 MHz, DMSO-d₆) δ=9.11 (t, J=6.0 Hz, 1H), 9.01 (d, J=1.6 Hz, 1H), 8.67 (d, J=2.0 Hz, 1H), 8.62 (dd, J=4.8, 2.0 Hz, 1H), 8.58 (d, J=2.0 Hz, 1H), 8.21 (dt, J=8.0, 1.6 Hz, 1H), 8.01 (dd, J=4.8, 1.6 Hz, 1H), 7.79 (s, 1H), 7.77-7.73 (m, 1H), 7.56-7.52 (m, 1H), 7.50-7.38 (m, 6H), 7.28 (dd, J=7.6, 1.6 Hz, 1H), 7.02 (s, 2H), 6.43 (dd, J=7.6, 4.8 Hz, 1H), 4.58 (d, J=6.0 Hz, 2H), 3.58 (s, 3H), 2.95-2.88 (m, 2H), 2.71-2.67 (m, 2H). HPLC Rₜ=4.405 min in 8 min chromatography, purity 99.3%. LCMS Rₜ=0.903 min in 2 min chromatography, purity 96.9%, MS ESI calcd, for 583.23 [M+H]⁺ 584.23, found 584.0.

### Example 94. Synthesis of 3-(3-((4-(2-(2-aminopyridin-3-yl)-6-(pyridin-3-yl)-3H-imidazo[4,5-b]pyridin-3-yl)benzyl)carbamoyl)phenyl) propanoic acid (Compound 94)

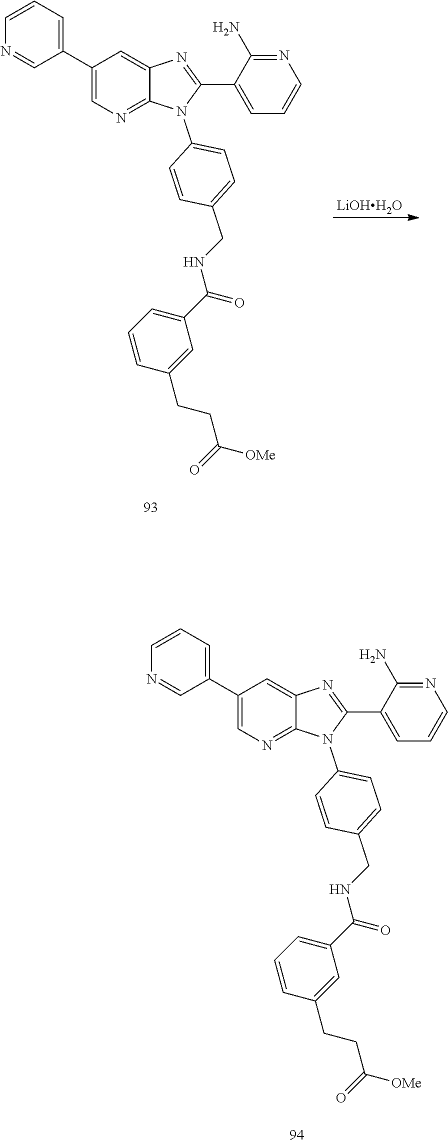 [SMILES: COC(=O)CCc1cccc(C(=O)NCc2ccc(-n3c(-c4cccnc4N)nc4cc(-c5cccnc5)cnc43)cc2)c1 ; "LiOH·H2O" ; COC(=O)CCc1cccc(C(=O)NCc2ccc(-n3c(-c4cccnc4N)nc4cc(-c5cccnc5)cnc43)cc2)c1]

Step 1. To a solution of methyl 3-(3-((4-(2-(2-aminopyridin-3-yl)-6-(pyridin-3-yl)-3H-imidazo[4,5-b]pyridin-3-yl)benzyl)carbamoyl)phenyl) propanoate 93 (200 mg, 0.343 mmol) in THF (3.00 mL) and water (3.00 mL), LiOH·H₂O (43.0 mg, 1.03 mmol) was added at 25° C., and the mixture was stirred for 16 hrs. The residue was purified by prep-HPLC (Column: Welch Xtimate C18 150*30 mm*5 μm; Condition: water (FA)-ACN; Begin B: 0-38%) to give 3-(3-((4-(2-(2-aminopyridin-3-yl)-6-(pyridin-3-yl)-3H-imidazo[4,5-b]pyridin-3-yl)benzyl)carbamoyl)phenyl) propanoic acid 94 (38.0 mg, 0.0669 mmol, 19.5% yield) as a white solid. ¹H NMR (400 MHz, DMSO-d₆) δ=9.10 (t, J=5.2 Hz, 1H), 9.01 (d, J=2.0 Hz, 1H), 8.67 (d, J=2.0 Hz, 1H), 8.62 (dd, J=4.8, 1.2 Hz, 1H), 8.58 (d, J=2.0 Hz, 1H), 8.23-8.18 (m, 1H), 8.01 (dd, J=4.8, 1.2 Hz, 1H), 7.80 (s, 1H), 7.77-7.72 (m, 1H), 7.54 (dd, J=8.0, 4.8 Hz, 1H), 7.50-7.36 (m, 6H), 7.28 (dd, J=7.6, 1.6 Hz, 1H), 7.02 (s, 2H), 6.43 (dd, J=7.6, 4.8 Hz, 1H), 4.58 (d, J=5.6 Hz, 2H), 2.88 (t, J=7.6 Hz, 2H), 2.58 (t, J=7.6 Hz, 2H). HPLC Rₜ=3.814 min in 8 min chromatography, purity 99.5%. LCMS Rₜ=1.844 min in 4 min chromatography, purity 98.6%, MS ESI calcd, for 569.22 [M+H]⁺ 570.22, found 570.5.

### Example 95. Synthesis of 2-(4-((4-(2-(2-aminopyridin-3-yl)-5-(pyridin-3-yl)-3H-imidazo[4,5-b]pyridin-3-yl)benzyl)carbamoyl)phenyl) acetic acid (Compound 95)

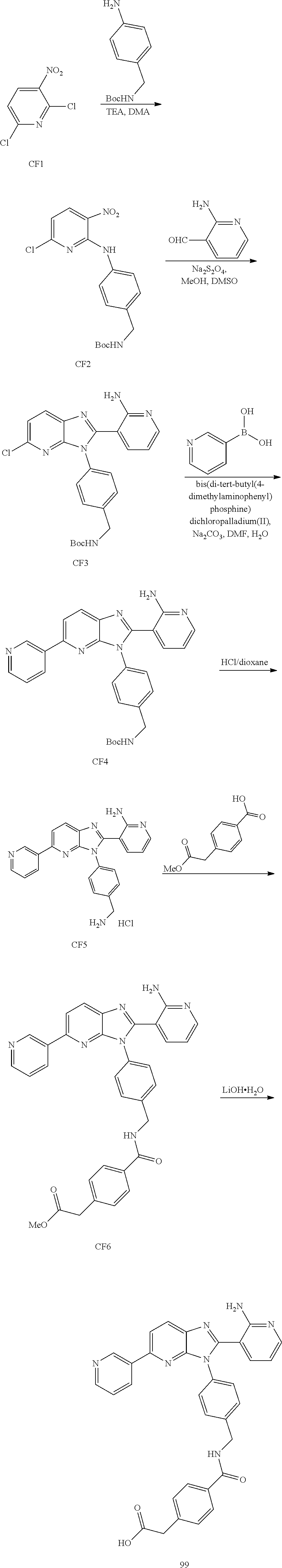

Step 1. To a solution of 2,6-dichloro-3-nitro-pyridine CF1 (2.00 g, 10.4 mmol) in DMA (10.0 mL), TEA (3.10 g, 31.1 mmol) was added. A solution of tert-butyl N-[(4-aminophenyl)methyl]carbamate (2.30 g, 10.4 mmol) in DMA (5.00 mL) was then added to the reaction mixture at 0° C., and the resulting mixture was stirred at 20° C. under N₂ for 12 hrs. The reaction mixture was diluted with EtOAc (20 mL), washed with water (50 mL and brine (50 mL×3), dried over anhydrous sodium sulfate, filtered and the filtrate was concentrated. The residue was purified by flash chromatography on silica gel (50% of EtOAc in PE) to afford tert-butyl N-[[4-[(6-chloro-3-nitro-2-pyridyl)amino]phenyl]methyl]carbamate CF2 (2.00 g, 5.28 mmol, 50.9% yield) as a yellow solid. LCMS Rₜ=0.659 min in 1 min chromatography, purity 100%, MS ESI calcd, for 378.11 [M+Na]⁺ 401.11, found 401.0.

Step 2. To a solution of 2-aminopyridine-3-carbaldehyde (870 mg, 7.13 mmol) and tert-butyl N-[[4-[(6-chloro-3-nitro-2-pyridyl)amino]phenyl]methyl]carbamate CF2 (2.00 g, 5.28 mmol) in DMSO (15.0 mL) and methanol (15.0 mL), Na₂S₂O₄ (2.70 g, 15.8 mmol) was added and the reaction mixture was stirred at 100° C. under N₂ for 12 hrs. The reaction mixture was concentrated to remove MeOH, subsequently diluted with EtOAc (30 mL), washed with brine (50 ml×2), dried over anhydrous sodium sulfate, filtered and the filtrate was concentrated. The residue was purified by flash chromatography on silica gel (90-100% of EtOAc in PE) to give tert-butyl N-[4-[2-(2-amino-3-pyridyl)-5-chloro-imidazo[4,5-b]pyridin-3-yl]phenyl]methyl]carbamate CF3 (500 mg, 1.11 mmol, 21.0% yield) as a yellow solid. ¹H NMR (400 MHz, DMSO-d₆) δ=8.27 (s, 1H), 7.99 (dd, J=2.0, 4.8 Hz, 1H), 7.45 (d, J=8.4 Hz. 2H), 7.38 (s, 4H), 7.21 (dd, J=2.0, 7.6 Hz, 1H), 6.91 (s, 2H), 6.40 (dd, J=4.8, 8.0 Hz, 1H), 4.22 (d, J=6.0 Hz, 2H), 1.41 (s, 9H).

Step 3. To a solution of tert-butyl N-[[4-[2-(2-amino-3-pyridyl)-5-chloro-imidazo[4,5-b]pyridin-3-yl]phenyl]methyl]carbamate CF3 (400 mg, 0.887 mmol) in DMF (9.00 mL) and water (0.180 mL), 3-pyridylboronic acid (158 mg, 1.29 mmol), Na₂CO₃ (103 mg. 0.97 mmol) and bis(di-tert-butyl (4-dimethylaminophenyl)phosphine)dichloropalladium (II) (63.0 mg, 0.0890 mmol) were added, and the reaction mixture was degassed and purged with N₂ (×3). The mixture was subjected to microwave irradiation at 160° C. for 1 hr. The mixture was subsequently cooled to 25° C. and diluted with EtOAc (30 mL), washed with brine (30 ML×3), dried over Na₂SO₄, then filtrated. The filtrate was concentrated and the resulting crude product was purified by flash chromatography on silica gel (100% of EtOAc in PE) to give tert-butyl N-[[4-[2-(2-amino-3-pyridyl)-5-(3-pyridyl) imidazo[4,5-b]pyridin-3-yl]phenyl]methyl]carbamate CF4 (200 mg, 0.410 mmol, 45.7% yield) as a brown oil. ¹H NMR (400 MHz, DMSO-d₆) δ=9.23 (d, J=2.0 Hz, 1H), 8.59 (dd, J=1.6, 4.8 Hz, 1H), 8.37 (td, J=2.0, 8.0 Hz, 1H), 8.32 (d, J=8.4 Hz, 1H), 8.08 (d, J=8.4 Hz, 1H), 8.01 (dd, J=2.0, 4.8 Hz, 1H), 7.65-7.35 (m, 5H), 7.21 (dd, J=1.6, 7.6 Hz, 1H), 7.00 (s, 2H), 6.76 (d, J=7.2 Hz, 1H), 6.42 (dd, J=4.8, 7.6 Hz, 1H), 4.24 (d, J=6.0 Hz, 2H), 1.41 (s, 9H).

Step 4. To a stirred solution of tert-butyl N-[[4-[2-(2-amino-3-pyridyl)-5-(3-pyridyl) imidazo[4,5-b]pyridin-3-yl]phenyl]methyl]carbamate CF4 (200 mg, 0.410 mmol) in 1,4-dioxane (2.00 mL), HCl/dioxane (1.00 mL, 4 M) was added and the reaction mixture was stirred at 20° C. under N₂ for 2 hrs. The reaction mixture was concentrated to afford 3-[3-[4-(aminomethyl)phenyl]-5-(3-pyridyl) imidazo[4,5-b]pyridin-2-yl]pyridin-2-amine hydrochloride CF5 (160 mg, crude) as a yellow solid. ¹H NMR (400 MHz, DMSO-d₆) δ=9.37 (s, 1H), 8.92-8.71 (m, 2H), 8.64-8.46 (m, 4H), 8.37 (s, 1H), 8.29-8.26 (m, 1H), 8.16 (d, J=6.0 Hz, 1H), 8.01-7.82 (m, 2H), 7.78-7.62 (m, 4H), 6.95-6.85 (m, 1H), 6.80 (d, J=8.0 Hz, 1H), 4.24-4.10 (m, 2H).

Step 5. To a stirred solution of 4-(2-methoxy-2-oxo-ethyl)benzoic acid (60.0 mg, 0.310 mmol) in DMF (2.00 mL), DIEA (98.0 mg, 0.762 mmol) and HATU (116 mg, 0.310 mmol) were added. After stirring at 20° C. for 10 mins, 3-[3-[4-(aminomethyl)phenyl]-5-(3-pyridyl) imidazo[4,5-b]pyridin-2-yl]pyridin-2-amine CF5 (100 mg, 0.250 mmol) was added to the reaction mixture, and the resulting mixture was stirred at 20° C. under N₂ for 2 hrs. The reaction mixture was diluted with EtOAc (10 mL), washed with water (20 mL), brine (20 mL×2), dried over anhydrous sodium sulfate, filtered and the filtrate was concentrated to afford methyl 2-(4-((4-(2-(2-aminopyridin-3-yl)-5-(pyridin-3-yl)-3H-imidazo[4,5-b]pyridin-3-yl)benzyl)carbamoyl)phenyl)acetate CF6 (100 mg, 0.176 mmol, 69.1% yield) as a yellow solid. LCMS Rₜ=1.617 min in 4 min chromatography, purity 70.4%, MS ESI calcd, for 569.22 [M+H]⁺ 570.22, found 570.2.

Step 6. To a stirred solution of methyl 2-(4-((4-(2-(2-aminopyridin-3-yl)-5-(pyridin-3-yl)-3H-imidazo[4,5-b]pyridin-3-yl)benzyl)carbamoyl)phenyl)acetate CF6 (100 mg, 0.180 mmol) in THF (2.00 mL) and water (1.00 mL), LiOH·H₂O (10 mg, 0.25 mmol) was added and the reaction mixture was stirred at 20° C. under N₂ for 2 hrs. The reaction mixture was concentrated directly and the resulting residue was purified by prep-HPLC (column: welch xtimate c18 150*25 mm*5 μm, method: water (NH₄HCO₃)-ACN, begin B: 10, end B: 40) to afford 2-(4-((4-(2-(2-aminopyridin-3-yl)-5-(pyridin-3-yl)-3H-imidazo[4,5-b]pyridin-3-yl)benzyl)carbamoyl)phenyl) acetic acid 95 (15.0 mg, 0.0260 mmol, 14.9% yield) as a light yellow solid. ¹H NMR (400 MHz, DMSO-d₆) δ=12.41 (s, 1H), 9.22 (d, J=2.0 Hz, 1H), 9.12 (1, J=6.0 Hz, 1H), 8.58 (dd, J=1.6, 4.8 Hz, 1H), 8.37 (td, J=2.0, 8.0 Hz, 1H), 8.31 (d, J=8.4 Hz, 1H), 8.07 (d, J=8.4 Hz, 1H), 8.01 (dd, J=2.0, 4.8 Hz, 1H), 7.88 (d, J=8.4 Hz, 2H), 7.55-7.44 (m, 5H), 7.37 (d, J=8.4 Hz, 2H), 7.25 (dd, J=2.0, 7.6 Hz, 1H), 6.99 (s, 2H), 6.44 (dd, J=4.8, 7.6 Hz, 1H), 4.61 (d, J=6.0 Hz, 2H), 3.65 (s, 2H). HPLC Rₜ=6.422 min in 15 min chromatography, purity 95.4%. LCMS Rₜ=0.920 min in 3 min chromatography, purity 99.4%, MS ESI calcd, for 555.20 [M+H]⁺ 556.20, found 556.2.

### Example 96. Synthesis of 2-(3-((4-(2-(2-aminopyridin-3-yl)-5-(pyridin-3-yl)-3H-imidazo[4,5-b]pyridin-3-yl)benzyl)carbamoyl)phenyl) acetic acid (Compound 96)

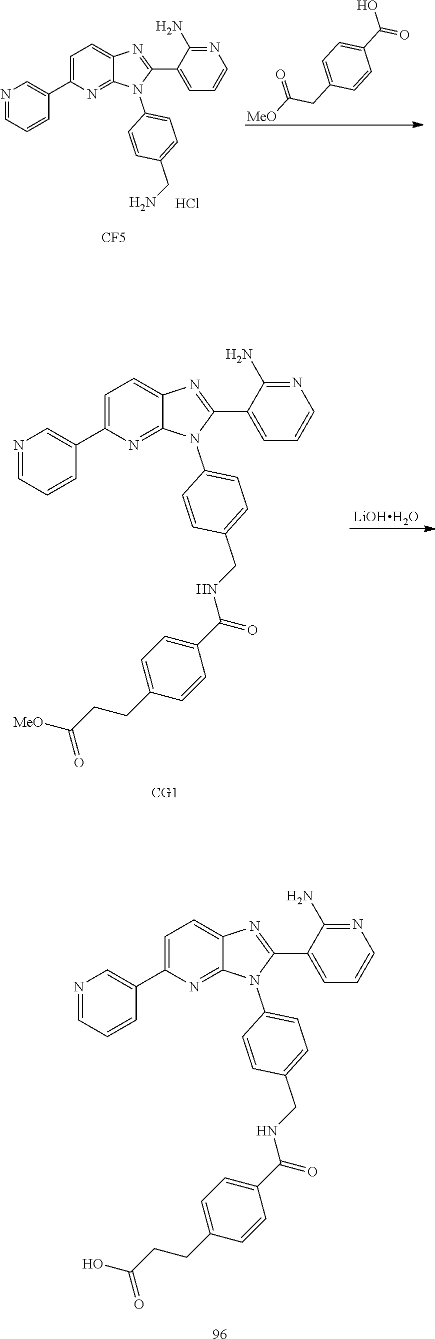

Step 1. To a stirred solution of 3-(2-methoxy-2-oxo-ethyl)benzoic acid (80.0 mg, 0.410 mmol) in DMF (2.00 mL), DIEA (0.0800 ml, 1.14 mmol) and HATU (145 mg, 0.380 mmol) were added. 3-[3-[4-(Aminomethyl)phenyl]-5-(3-pyridyl) imidazo[4,5-b]pyridin-2-yl]pyridin-2-amine CF5 (150 mg, 0.380 mmol) was then added to the reaction mixture, and the resulting mixture was stirred at 25° C. under N₂ for 2 hrs. The reaction mixture was diluted with EtOAc (10 mL) and washed with brine (10 mL×3), dried over anhydrous sodium sulfate, filtered and the filtrate was concentrated and purified by flash chromatography on silica gel (5% of MeOH in DCM) to afford methyl 2-(3-((4-(2-(2-aminopyridin-3-yl)-5-(pyridin-3-yl)-3H-imidazo[4,5-b]pyridin-3-yl)benzyl)carbamoyl)phenyl)acetate CG1 (100 mg, 0.180 mmol, 46.1% yield) as a yellow solid. ¹H NMR (400 MHz, DMSO-d₆) δ=9.32-9.07 (m, 2H), 8.58 (dd, J=1.6, 4.8 Hz, 1H), 8.37 (td, J=2.0, 8.0 Hz, 1H), 8.33-8.21 (m, 1H), 8.08 (d, J=8.4 Hz, 1H), 8.01 (dd, J=2.0, 4.8 Hz, 1H), 7.84-7.81 (m, 2H), 7.77-7.68 (m, 1H), 7.52-7.43 (m, 8H), 7.24 (dd, J=2.0, 7.6 Hz, 1H), 6.97 (d, J=4.0 Hz, 1H), 6.43 (dd, J=4.8, 7.6 Hz, 1H), 4.60 (d, J=6.0 Hz, 2H), 3.67-3.60 (m, 4H).

Step 2. To a stirred solution of methyl 2-(3-((4-(2-(2-aminopyridin-3-yl)-5-(pyridin-3-yl)-3H-imidazo[4,5-b]pyridin-3-yl)benzyl)carbamoyl)phenyl)acetate CG1 (100 mg, 0.180 mmol) in THF (1.00 mL) and water (1.00 mL), LiOH (14.0 mg, 0.350 mmol) was added and the reaction mixture was stirred at 20° C. under N₂ for 12 hrs. The reaction mixture was concentrated and purified by prep-HPLC (column: Welch Xtimate C18 150*30 mm*5 mm, method: water (FA)-ACN, begin B: 7, end B: 37) to afford 2-(3-((4-(2-(2-aminopyridin-3-yl)-5-(pyridin-3-yl)-3H-imidazo[4,5-b]pyridin-3-yl)benzyl)carbamoyl)phenyl) acetic acid 96 (45.0 mg, 0.0790 mmol, 44.9% yield) as a light yellow solid. ¹H NMR (400 MHz, DMSO-d₆) δ=12.46 (s, 1H), 9.23 (d, J=2.0 Hz, 1H), 9.20-9.09 (m, 1H), 8.58 (dd, J=1.6, 4.8 Hz, 1H), 8.37 (td, J=2.0, 8.0 Hz, 1H), 8.32 (d, J=8.4 Hz, 1H), 8.07 (d, J=8.4 Hz, 1H), 8.01 (dd, J=2.0, 4.8 Hz, 1H), 7.90-7.74 (m, 2H), 7.61-7.36 (m, 7H), 7.24 (dd, J=2.0, 7.6 Hz, 1H), 6.99 (s, 2H), 6.43 (dd, J=4.8, 7.6 Hz, 1H), 4.60 (d, J=6.0 Hz, 2H), 3.71-3.61 (m, 2H). HPLC Rₜ=2.146 min in 8 min chromatography, purity 97.2%. LCMS Rₜ=1.333 min in 3 min chromatography, purity 96.9%, MS ESI calcd, for 555.20 [M+H]⁺ 556.20, found 556.3.

### Example 97. Synthesis of 2-[3-[[4-[2-(2-amino-3-pyridyl)-6-carbamoyl-imidazo[4,5-b]pyridin-3-yl]phenyl]methylcarbamoyl]phenyl]acetic acid (Compound 97)

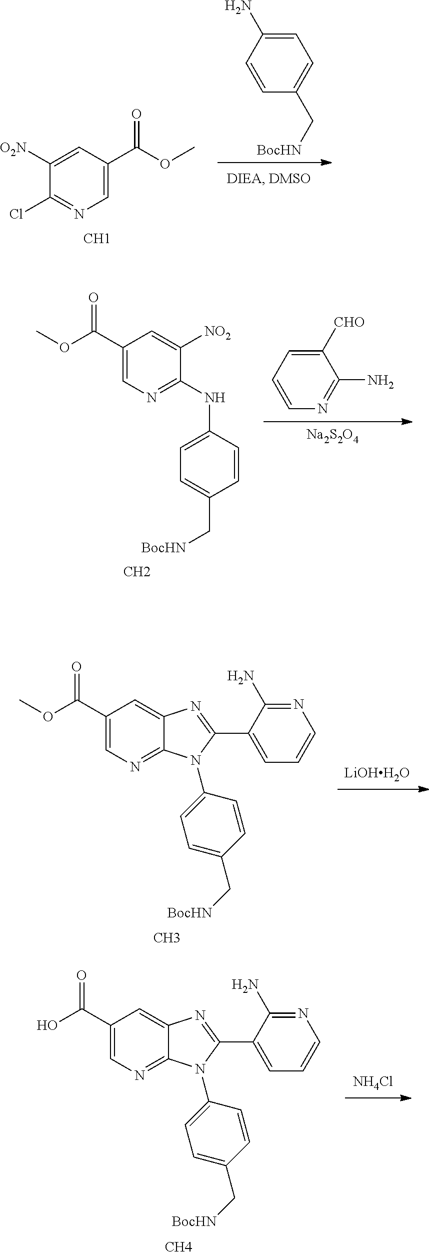

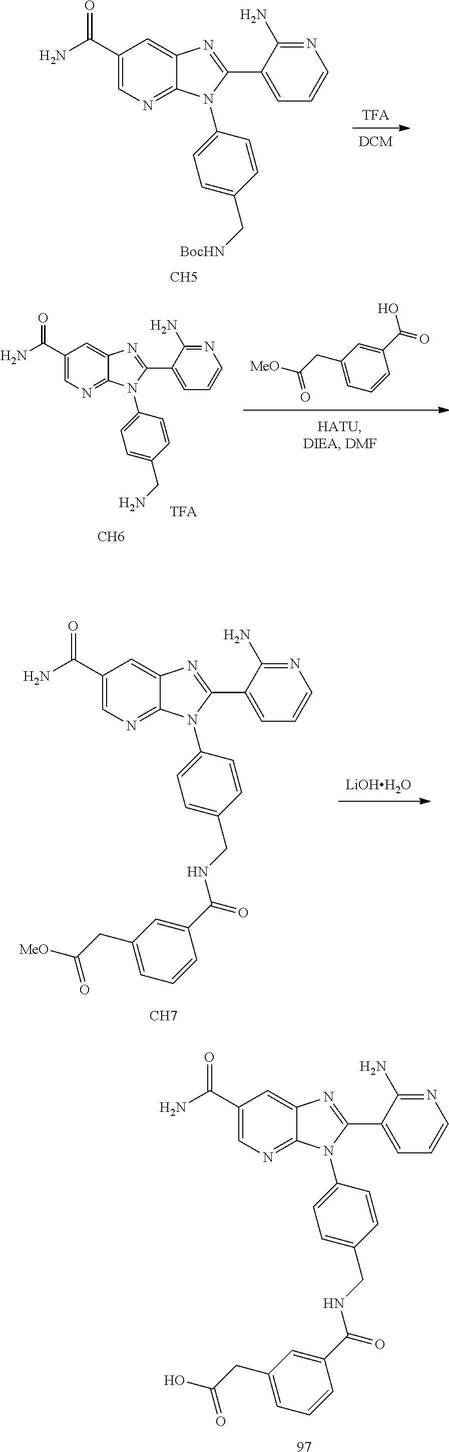

Step 1. To a solution of methyl 6-chloro-5-nitro-pyridine-3-carboxylate CH1 (5.00 g, 23.1 mmol) in DMSO (80.0 mL), DIEA (8.95 g, 69.3 mmol) and tert-butyl N-[(4-aminophenyl)methyl]carbamate (6.16 g, 27.7 mmol) were added and the mixture was stirred at 80° C. for 12 hrs. The reaction mixture was filtered, and the filter cake was concentrated to give methyl 6-[4-[(tert-butoxycarbonylamino)methyl]anilino]-S-nitro-pyridine-3-carboxylate CH2 (9.00 g, 96.9% yield) as a yellow solid, which was used in the next step without purification. ¹H NMR (400 MHz, DMSO-d₆) δ=10.29 (s, 1H), 8.89 (d, J=2.0 Hz, 1H), 8.81 (d, J=2.0 Hz, 1H), 7.53 (d, J=8.4 Hz, 2H), 7.41 (t, J=6.4 Hz, 1H), 7.26 (d, J=8.4 Hz, 2H), 4.13 (d, J=6.0 Hz, 2H), 3.87 (s, 3H), 1.40 (s, 9H).

Step 2. To a solution of methyl 6-[4-[(tert-butoxycarbonylamino)methyl]anilino]-5-nitro-pyridine-3-carboxylate CH2 (3.50 g, 8.70 mmol) in DMSO (50.0 mL) and MeOH (20.0 mL), 2-aminopyridine-3-carbaldehyde (1.06 g, 8.70 mmol) and Na₂S₂O₄ (4.54 g, 26.1 mmol) were added and the reaction mixture was concentrated. Aq. LiCl (100 mL) was added to the residue, and the resulting mixture was extracted with EtOAc (100 mL×3). The combined organic phase was washed with brine (100 mL), dried over anhydrous Na₂SO₄, filtered and concentrated. The residue was purified by flash silica gel chromatography (eluent: 48%-67%) of EtOAc in PE) to give methyl 2-(2-amino-3-pyridyl)-3-[4-[(tert-butoxycarbonylamino)methyl]phenyl]imidazo[4,5-b]pyridine-6-carboxylate CH3 (2.25 g. 54.5% yield) as a yellow solid. ¹H NMR (400 MHz, CHLOROFORM-d) δ=9.10-9.02 (m, 1H), 8.76-8.71 (m, 1H), 8.01 (d, J=5.2 Hz, 1H), 7.54-7.47 (m, 4H), 7.37-7.34 (m, 2H), 7.26-7.26 (m, 1H), 7.27-7.21 (m, 1H), 6.48-6.39 (m, 1H), 4.46-4.42 (m, 2H), 3.99 (s, 3H), 1.48 (s, 9H). LCMS Rₜ=0.538 min in 1 min chromatography, purity 91.3%, MS ESI calcd. for 474.20 [M+H]⁺ 475.20, found 475.3.

Step 3. To a solution of methyl 2-(2-amino-3-pyridyl)-3-[4-[(tert-butoxycarbonylamino)methyl]phenyl]imidazo[4,5-b]pyridine-6-carboxylate CH3 (2.25 g, 4.74 mmol) in THF (10.0 mL) and water (10.0 mL), LiOH·H₂O (214 mg, 5.22 mmol) was added and the mixture was stirred at 25° C. for 2 hrs. The reaction mixture was concentrated directly to give 2-(2-amino-3-pyridyl)-3-[4-[(tert-butoxycarbonylamino)methyl]phenyl]imidazo[4,5-b]pyridine-6-carboxylic acid CH4 (1.65 g, crude) as a brown solid. LCMS Rₜ=0.468 min in 1 min chromatography, purity 94.6%, MS ESI calcd, for 460.19 [M+H]⁺ 461.19, found 461.2.

Step 4. To a solution of 2-(2-aminopyridin-3-yl)-3-(4-(((tert-butoxycarbonyl)amino)methyl)phenyl)-3H-imidazo[4,5-b]pyridine-6-carboxylic acid CH4 (1.63 g, 3.55 mmol) and NH₄Cl (1.90 g, 35.5 mmol) in DMF (15.0 mL), DIEA (1.38 g, 10.6 mmol) and HATU (1.75 g. 4.61 mmol) were added and the mixture was stirred at 25° C. for 12 hrs. Water (50 mL) was added, and the resulting mixture was extracted with EtOAc (50 ml×3). The combined organic phase was washed with brine (50 mL), dried over anhydrous Na₂SO₄, filtered and concentrated. The residue was purified by flash silica gel chromatography (eluent: 0%-6% of MeOH in DCM) to give tert-butyl N-[[4-[2-(2-amino-3-pyridyl)-6-carbamoyl-imidazo[4,5-b]pyridin-3-yl]phenyl]methyl]carbamate CH5 (1.18 g. 72.4% yield) as a yellow oil. ¹H NMR (400 MHz, DMSO-d₆) δ=8.85-8.82 (m, 1H), 8.66-8.63 (s, 1H), 8.21-8.16 (m, 1H), 8.00 (dd, J=1.6, 4.8 Hz, 1H), 7.55 (s, 1H), 7.50-7.46 (m, 1H), 7.42-7.35 (m, 4H), 7.27-7.22 (m, 1H), 6.99-6.90 (m, 2H), 6.46-6.37 (m, 1H), 4.24-4.18 (m, 2H), 1.41 (s, 9H). LCMS Rₜ=0.535 min in 1 min chromatography, purity 87.6%, MS ESI calcd, for 459.20 [M+H]⁺ 460.20, found 460.2.

Step 5. To a solution of tert-butyl (4-(2-(2-aminopyridin-3-yl)-6-carbamoyl-3H-imidazo[4,5-b]pyridin-3-yl)benzyl)carbamate CH5 (1.18 g, 2.54 mmol) in DCM (4.00 mL), TFA (1.00 mL, 1.47 mmol) was added, and the mixture was stirred at 25° C. for 12 hrs. The reaction mixture was concentrated directly to give 3-[4-(aminomethyl)phenyl]-2-(2-amino-3-pyridyl) imidazo[4,5-b]pyridine-6-carboxamide trifluoroacetate CH6 (600 mg, 65.0% yield) as a brown oil, which was used in the next step without further purification.

Step 6. To a solution of 3-[4-(aminomethyl)phenyl]-2-(2-amino-3-pyridyl) imidazo[4,5-b]pyridine-6-carboxamide trifluoroacetate CH6 (100 mg, 0.280 mmol) and 3-(2-methoxy-2-oxo-ethyl)benzoic acid (59.0 mg, 0.310 mmol) in DMF (2.00 mL), HATU (159 mg, 0.417 mmol) and DIEA (54.0 mg, 0.417 mmol) were added and the mixture was stirred at 25° C. for 2 hrs. The reaction mixture was concentrated directly. Sat. aq. LiCl (10 mL) was added, and the resulting mixture was extracted with EtOAc (10 mL×3). The combined organic phase was washed with brine (10 mL), dried over anhydrous Na₂SO₄, filtered and concentrated to give methyl 2-(3-((4-(2-(2-aminopyridin-3-yl)-6-carbamoyl-3H-imidazo[4,5-b]pyridin-3-yl)benzyl)carbamoyl)phenyl)acetate CH7 (142 mg, 95.3% yield) as a yellow oil. ¹H NMR (400 MHz, CHLOROFORM-d) δ=10.77-10.66 (m, 5H), 9.00-8.87 (m, 1H), 8.60-8.55 (m, 1H), 7.88-7.84 (m, 1H), 7.78-7.74 (s, 2H), 7.64-7.60 (d, J=8.0 Hz, 2H), 7.46-7.44 (m, 2H), 7.41-7.38 (m, 2H), 6.83-6.75 (m, 1H), 6.56-6.50 (m, 1H), 4.79 (d, J=5.6 Hz, 2H), 3.71 (s, 3H), 3.70 (s, 2H). LCMS Rₜ=0.505 min in 1 min chromatography, purity 86.4%, MS ESI calcd, for 535.20 [M+H]⁺ 536.20, found 536.4.

Step 7. To a solution of methyl 2-(3-((4-(2-(2-aminopyridin-3-yl)-6-carbamoyl-3H-imidazo[4,5-b]pyridin-3-yl)benzyl)carbamoyl)phenyl)acetate CH7 (142 mg, 0.260 mmol) in THF (2.00 mL), water (2.00 mL) and LiOH·H₂O (14.0 mg, 0.336 mmol) were added and the mixture was stirred at 25° C. for 2 hrs. The reaction mixture was concentrated directly and the resulting residue was purified by prep HPLC (Column: Xtimate C18 150*40 mm*10 μm mobile phase: [water (HCl)-ACN]; B %: 0%-30%; 25 min) to give 2-(3-((4-(2-(2-aminopyridin-3-yl)-6-carbamoyl-3H-imidazo[4,5-b]pyridin-3-yl)benzyl)carbamoyl)phenyl) acetic acid 97 (22.5 mg, 16.3% yield) as a white solid. ¹H NMR (400 MHz, DMSO-d₆) δ=9.19-9.10 (m, 1H), 8.92 (d, J=2.0 Hz, 1H), 8.73 (d, J=2.0 Hz, 1H), 8.58-8.16 (m, 3H), 8.14-8.09 (m, 1H), 7.98-7.92 (m, 1H), 7.84-7.79 (m, 2H), 7.60 (s, 1H), 7.48 (s, 4H), 7.46-7.42 (m, 2H), 6.94-6.86 (m, 1H), 4.56 (d, J=6.0 Hz, 2H), 3.65 (s, 2H). HPLC Rₜ=2.375 min in 8 min chromatography, purity 99.5%. LCMS Rₜ=1.005 min in 2.5 min chromatography, purity 96.7%, MS ESI calcd, for $21.18 [M+H]⁺ 522.18, found 522.2.

### Example 98. Synthesis of 4-((4-(2-(2-aminopyridin-3-yl)-6-(trifluoromethyl)-3H-imidazo[4,5-b]pyridin-3-yl)benzyl)carbamoyl)benzoic acid (Compound 98)

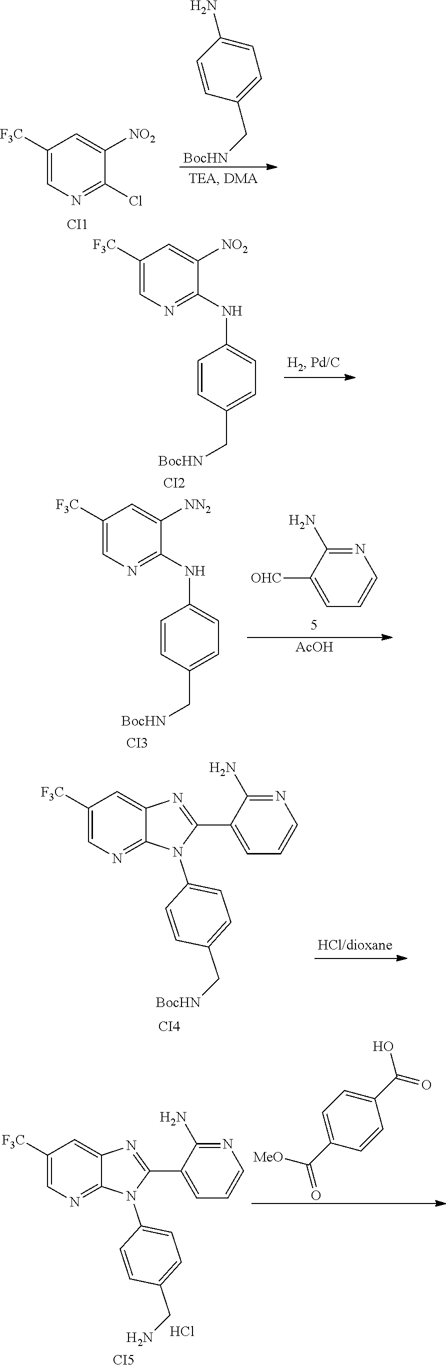

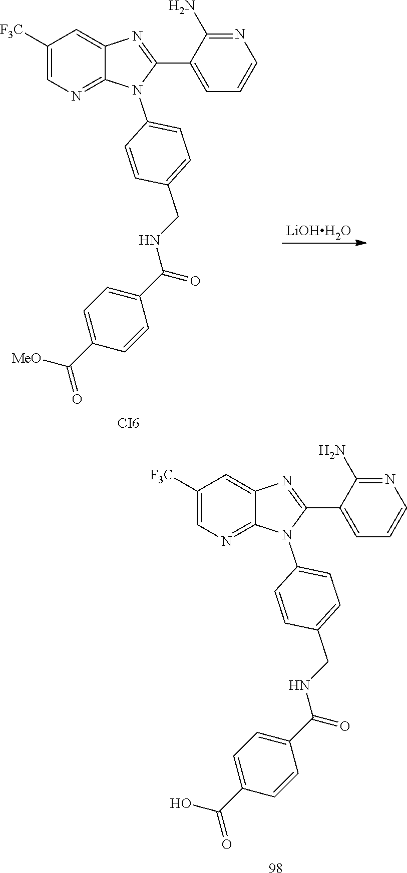

Step 1. To a solution of 2-chloro-3-nitro-5-(trifluoromethyl)pyridine CI1 (2.00 g, 8.83 mmol) in DMA (20.0 mL), tert-butyl N-[(4-aminophenyl)methyl]carbamate (2.35 g, 10.6 mmol) and TEA (2.68 g, 26.5 mmol) were added and the resulting mixture was stirred at 80° C. for 12 hrs. The mixture was diluted with H₂O (10 mL), extracted with EtOAc (20 mL×3), and washed with brine (30 mL). The organic layer was dried over Na₂SO₄, filtered, and concentrated. The residue was purified by flash silica gel chromatography (ISCO®; 80 g SepaFlash® Silica Flash Column, Eluent of 11% of Ethyl acetate in Petroleum ether gradient @100 mL/min) to give tert-butyl N-[[4-[[3-nitro-5-(trifluoromethyl)-2-pyridyl]amino]phenyl]methyl]carbamate CI2 (4.84 g, crude) as a red solid. LCMS Rₜ=1.745 min in 2.5 min chromatography, purity 21.4%, MS ESI calcd, for 412.14 [M+H]⁺ 413.14, found 413.0.

Step 2. To a solution of tert-butyl N-[[4-[3-nitro-5-(trifluoromethyl)-2-pyridyl]amino]phenyl]methyl]carbamate CI2 (4.84 g, 11.7 mmol) in THF (50.0 mL), Pd/C (500 mg, 10%, wet) was added and the reaction mixture was stirred under H₂ (15 psi) at 25° C. for 2 hrs. The mixture was filtered via a celite pad, and the filter cake was washed with DCM/MeOH (1/1; 500 mL). The organic phase was concentrated to give tert-butyl N-[[4-[[3-amino-5-(trifluoromethyl)-2-pyridyl]amino]phenyl]methyl]carbamate CI3 (4.40 g, crude) as a brown solid, which was used in the next step without further purification. LCMS Rₜ=1.364 min in 2.5 min chromatography, purity 96.0%, MS ESI calcd, for 382.16 [M+H]⁺ 383.16, found 383.5.

Step 3. To a solution of tert-butyl N-[[4-[3-amino-5-(trifluoromethyl)-2-pyridyl]amino]phenyl]methyl]carbamate CI3 (300 mg, 0.780 mmol) in acetic acid (5.00 mL), 2-aminopyridine-3-carbaldehyde (144 mg, 1.18 mmol) was added and the reaction was stirred at 100° C. for 12 hrs. The reaction mixture was concentrated directly and the resulting residue was purified by flash silica gel chromatography (ISCOR; 12 g SepaFlash® Silica Flash Column, Eluent with 40% of Ethyl acetate in Petroleum ether gradient @ 50 mL/min) to give tert-butyl N-[[4-[2-(2-amino-3-pyridyl)-6-(trifluoromethyl) imidazo[4,5-b]pyridin-3-yl]phenylmethyl]carbamate CI4 (82.0 mg, 21.5% yield) as a light yellow solid. LCMS Rₜ=1.310 min in 2.5 min chromatography, purity 20.1%, MS ESI calcd, for 484.18 [M+H]⁺ 485.18, found 485.1.

Step 4. To a solution of tert-butyl N-[[4-[2-(2-amino-3-pyridyl)-6-(trifluoromethyl) imidazo[4,5-b]pyridin-3-yl]phenyl]methyl]carbamate CI4 (82.0 mg, 0.160 mmol) in DCM (2.00 mL), HCl-dioxane (2.00 mL, 4 M) was added and the reaction was stirred at 25° C. for 2 hrs. The reaction mixture was concentrated directly to give 3-[3-[4-(aminomethyl)phenyl]-6-(trifluoromethyl) imidazo[4,5-b]pyridin-2-yl]pyridin-2-amine hydrochloride CI5 (98.0 mg, crude), which was used in the next step without further purification. LCMS Rₜ=0.462 min in 1.5 min chromatography, purity 99.5%, MS ESI calcd. for 384.13 [M+H]⁺ 385.13, found 385.3.

Step 5. To a solution of Mar. 13, 2014-(aminomethyl)phenyl]-6-(trifluoromethyl) imidazo[4,5-b]pyridin-2-yl]pyridin-2-amine hydrochloride CI5 (98.0 mg, 0.250 mmol) in pyridine (5.00 mL), 4-methoxycarbonylbenzoic acid (69.0 mg, 0.380 mmol) and EDCI (31.0 mg, 0.760 mmol) were added and the resulting mixture was stirred at 25° C. for 2 hrs. The mixture was diluted with H₂O (10 mL), extracted with EtOAC (10 mL×3), washed with brine (30 mL). The organic layer was dried over Na₂SO₄, concentrated, and the resulting residue was purified by flash silica gel chromatography (ISCOR; 4 g SepaFlash® Silica Flash Column, Eluent with 70% of Ethyl acetate in Petroleum ether gradient @ 50 mL/min) to give methyl 4-((4-(2-(2-aminopyridin-3-yl)-6-(trifluoromethyl)-3H-imidazo[4,5-b]pyridin-3-yl)benzyl)carbamoyl)benzoate CI6 (90.0 mg, 64.5% yield) as a yellow solid. LCMS Rₜ=1.231 min in 2.5 min chromatography, purity 96.8%, MS ESI calcd, for 546.16 [M+H]⁺ 547.16, found 547.3.

Step 6. To a solution of methyl 4-((4-(2-(2-aminopyridin-3-yl)-6-(trifluoromethyl)-3H-imidazo[4,5-b]pyridin-3-yl)benzyl)carbamoyl)benzoate CI6 (90.0 mg, 0.160 mmol) in THF (2.00 mL), hydroxylithium; hydrate (34.0 mg, 0.820 mmol) and water (1.00 mL) were added and the reaction mixture was stirred at 25° C. for 2 hrs. The reaction mixture was concentrated to give the crude product. The mixture was diluted with H₂O (10 mL) and extracted with PE (10 mL×3). The pH of the aqueous phase was adjusted to pH ~5 with HCl (2 M). The crude product was purified by prep-HPLC (column: Welch Xtimate C18 40*200 mm, 7 μm; mobile phase: [water (FA)-ACN]; B %: 4%-44%, 25 min) to provide 4-((4-(2-(2-aminopyridin-3-yl)-6-(trifluoromethyl)-3H-imidazo[4,5-b]pyridin-3-yl)benzyl)carbamoyl)benzoic acid 98 (66.8 mg, 76.4% yield) as a white solid. ¹H NMR (DMSO-d₆, 400 MHz) δ=9.29 (d, J=7.6 Hz, 2H), 8.67 (s, 1H), 8.63 (s, 1H), 8.02 (q, J=4.0 Hz, 5H), 7.46 (q, J=8.4 Hz, 4H), 7.31 (dd, J=7.6, 2.0 Hz, 1H), 6.87 (s, 2H), 6.44 (dd, J=7.6, 4.8 Hz, 1H), 4.58 (d, J=6.0 Hz, 2H). HPLC R; =3.776 min in 8 min chromatography, purity 95.3%. LCMS Rₜ=2.171 min in 4 min chromatography, purity 99.2%, MS ESI calcd. for 532.15 [M+H]⁺ 533.15, found 533.1.

### Example 99. Synthesis of 4-((4-(2-(2-aminopyridin-3-yl)-5-(trifluoromethyl)-3H-imidazo[4,5-b]pyridin-3-yl)benzyl)carbamoyl)benzoic acid (Compound 99)

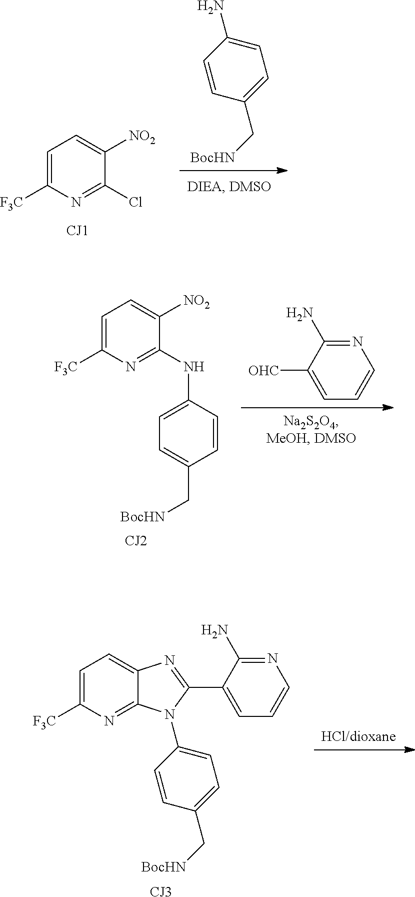

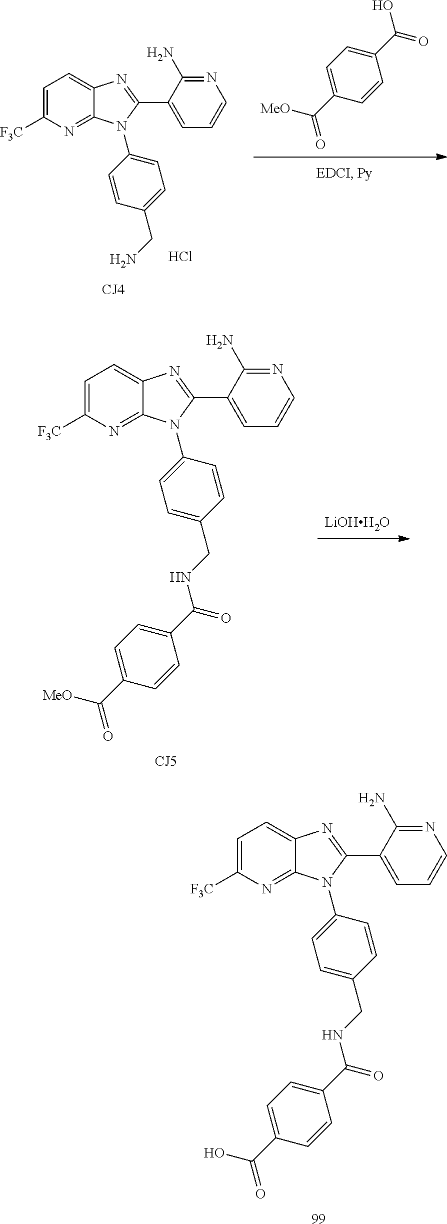

Step 1. To a stirred solution of 2-chloro-3-nitro-6-(trifluoromethyl)pyridine CJ1 (1.00 g, 4.41 mmol) and tert-butyl N-[(4-aminophenyl)methyl]carbamate (0.980 g, 4.41 mmol) in DMSO (20.0 mL), DIEA (2.40 mL, 13.2 mmol) was added and the reaction was stirred at 95° C. under N₂ for 16 hrs. The reaction mixture was diluted with EtOAc (50 mL), washed with water (50 mL) and brine (50 mL×2), dried over anhydrous sodium sulfate, filtered and the filtrate was concentrated to afford a residue. The crude product was purified by flash chromatography on silica gel (95% of EtOAc in PE) to afford tert-butyl N-[[4-[[3-nitro-6-(trifluoromethyl)-2-pyridyl]amino]phenyl]methyl]carbamate CJ2 (1.20 g, 65.9% yield) as a yellow solid. ¹H NMR (400 MHz, DMSO-d₆) δ=10.00 (s, 1H), 8.74 (d, J=8.4 Hz, 1H), 7.58 (d, J=8.4 Hz, 2H), 7.37 (d, J=8.4 Hz, 2H), 7.24 (d, J=8.3 Hz, 2H), 4.12 (d, J=6.0 Hz, 2H), 1.40 (s, 9H).

Step 2. To a stirred solution of 2-aminopyridine-3-carbaldehyde (400 mg, 3.27 mmol) and tert-butyl N-[4-[[3-nitro-6-(trifluoromethyl)-2-pyridyl]amino]phenyl]methyl]carbamate CJ2 (1.00 g, 2.43 mmol) in DMSO (15.0 mL) and methanol (1.00 mL), Na₂S₂O₄ (1.26 g, 7.28 mmol) was added and the reaction mixture was stirred at 90° C. under N₂ for 16 hrs. The reaction mixture was diluted with EtOAc (50 mL), washed with water (30 mL) and brine (50 mL×2), dried over anhydrous sodium sulfate, filtered and the filtrate was concentrated and purified by flash chromatography on silica gel (90% of EtOAc in PE) to afford tert-butyl N-[[4-[2-(2-amino-3-pyridyl)-5-(trifluoromethyl) imidazo[4,5-b]pyridin-3-yl]phenyl]methyl]carbamate CJ3 (150 mg, 12.7% yield) as a yellow solid. ¹H NMR (400 MHz, CDCl₃) δ=8.19 (d, J=8.4 Hz, 1H), 8.10 (dd, J=1.6, 4.8 Hz, 1H), 7.70 (d, J=8.4 Hz, 1H), 7.45 (d, J=8.4 Hz, 2H), 7.38-7.29 (m, 2H), 7.11 (d, J=8.0 Hz, 1H), 6.64 (s, 2H), 6.40 (dd, J=4.8, 7.6 Hz, 1H), 4.99 (s, 1H), 4.44 (d, J=4.8 Hz, 2H), 1.49 (s, 9H).

Step 3. To a stirred solution of tert-butyl N-[4-[2-(2-amino-3-pyridyl)-5~ (trifluoromethyl) imidazo[4,5-b]pyridin-3-yl]phenyl]methyl]carbamate CJ3 (150 mg, 0.310 mmol) in 1,4-dioxane (1.00 mL), HCl/dioxane (2.00 mL, 2 M) was added and the reaction mixture was stirred at 25° C. under N₂ for 16 hrs. The reaction mixture was concentrated to afford 3-[3-[4-(aminomethyl)phenyl]-5-(trifluoromethyl) imidazo[4,5-b]pyridin-2-yl]pyridin-2-amine hydrochloride CJ4 (100 mg, 84.0% yield) as a yellow solid. LCMS Rₜ=0.28 min in 1.0 min chromatography, purity 93.4%, MS ESI calcd, for 384.13 [M+H]⁺ 385.13, found 385.3.

Step 4. To a stirred solution of 3-[3-[4-(aminomethyl)phenyl]-5-(trifluoromethyl) imidazo[4,5-b]pyridin-2-yl]pyridin-2-amine hydrochloride CJ4 (100 mg, 0.260 mmol) and 4-methoxycarbonylbenzoic acid (47.0 mg, 0.260 mmol) in pyridine (3.00 mL), EDCI (55.0 mg, 0.280 mmol) was added and the reaction mixture was stirred at 25° C. under N₂ for 16 hrs. The reaction mixture was diluted with EtOAc (20 mL), washed with 1N HCl solution (15 mL×2), brine (50 mL), dried over anhydrous sodium sulfate, filtered and the filtrate was concentrated to afford methyl 4-((4-(2-(2-aminopyridin-3-yl)-5-(trifluoromethyl)-3H-imidazo[4,5-b]pyridin-3-yl)benzyl)carbamoyl)benzoate CJ5 (100 mg, 70.3% yield) as a yellow solid. LCMS Rₜ=0.46 min in 1.0 min chromatography, purity 56.9%, MS ESI calcd. for 546.16 [M+H]⁺ 547.16, found 547.2.

Step 5. To a stirred solution of methyl 4-((4-(2-(2-aminopyridin-3-yl)-5-(trifluoromethyl)-3H-imidazo[4,5-b]pyridin-3-yl)benzyl)carbamoyl)benzoate CJ5 (100 mg, 0.180 mmol) in THF (1.00 mL) and water (1.00 mL), LiOH·H₂O (15.0 mg, 0.370 mmol) was added and the reaction mixture was stirred at 25° C. under N₂ for 16 hrs. The reaction mixture was adjusted to pH ~5 with 1 M HCl and concentrated to afford a residue. The crude product was purified by prep-HPLC (column: welch xtimate C18 150×30 mm×5 mm, method: water (FA)-ACN, begin B: 23, end B: 53) to afford 4-(4-(2-(2-aminopyridin-3-yl)-5-(trifluoromethyl)-3H-imidazo[4,5-b]pyridin-3-yl)benzyl)carbamoyl)benzoic acid 99 (17.0 mg, 16.6% yield) as a white solid. ¹H NMR (400 MHz, DMSO-d₆) δ=13.24 (s, 1H), 9.30 (t, J=6.0 Hz, 1H), 8.43 (d, J=8.4 Hz, 1H), 8.07-7.99 (m, 5H), 7.87 (d, J=8.4 Hz, 1H), 7.57-7.38 (m, 4H), 7.29 (dd, J=2.0, 7.6 Hz, 1H), 6.84 (s, 2H), 6.44 (dd, J=4.8, 7.6 Hz, 1H), 4.60 (d, J=6.0 Hz, 2H). 1F NMR (400 MHz, DMSO-d₆) δ=−64.192. HPLC Rₜ=3.334 min in 8 min chromatography, purity 93.7%. LCMS Rₜ=1.830 min in 4 min chromatography, purity 99.2%, MS ESI calcd, for 532.15 [M+H]⁺ 533.15, found 533.1.

### Example 100. Synthesis of methyl 2-(3-((4-(2-(2-aminopyridin-3-yl)-3H-imidazo[4,5-b]pyridin-3-yl)benzyl)carbamoyl)phenyl)acetate (Compound 100)

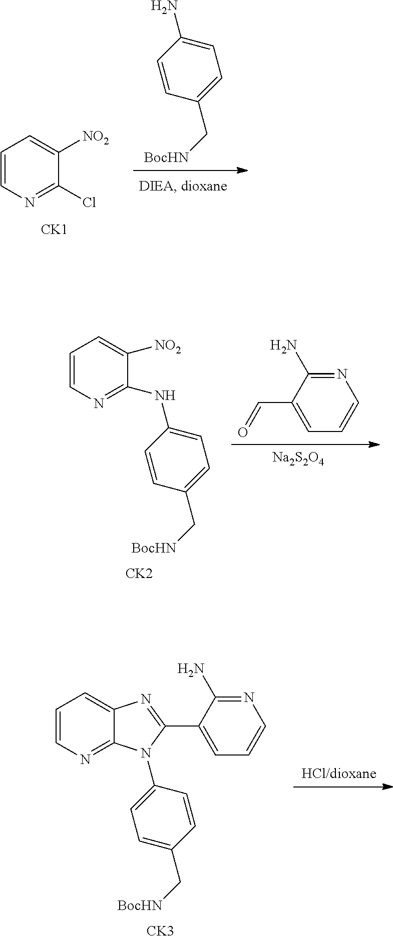

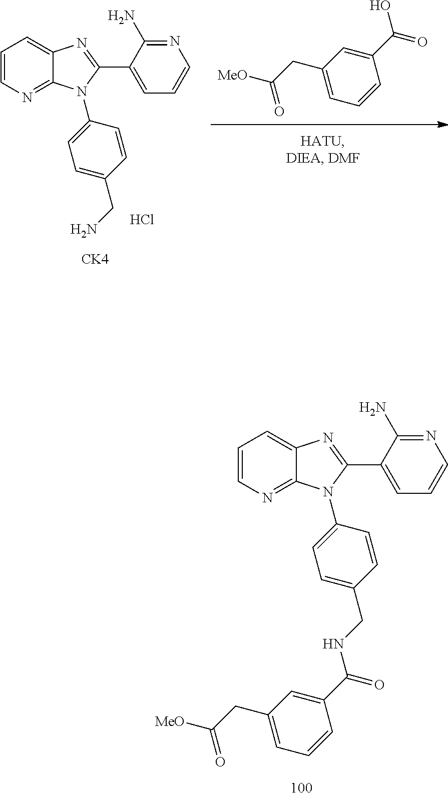

Step 1. To the solution of 2-chloro-3-nitro-pyridine CK1 (3.10 g, 19.5 mmol) in 1,4-dioxane (30.0 mL), tert-butyl N-[(4-aminophenyl)methyl]carbamate (4.34 g, 19.5 mmol) and DIEA (7.20 mL, 39.0 mmol) were added and the mixture stirred at 80° C. for 24 hrs. The reaction mixture was concentrated in vacuum, water (50 mL×3) was added and the whole extracted with EtOAc (80 mL×3). The organic layer was dried over Na₂SO₄, filtered and the filtrate was concentrated in vacuum. The crude residue was purified by flash column (0-20% of EtOAc in PE) to give tert-butyl (4-((3-nitropyridin-2-yl)amino)benzyl)carbamate CK₂ (1.20 g, 3.48 mmol, 17.8% yield) as a yellow solid. ¹H NMR (400 MHz, DMSO-d₆) δ_{H}=9.94 (s, 1H) 8.56-8.52 (m, 1H), 8.51-8.47 (m, 1H), 7.56 (d, J=8.4 Hz, 2H), 7.40 (t, J=6.0 Hz, 1H), 7.22 (d, J=8.0 Hz, 2H), 6.97 (dd, J=8.0, 4.4 Hz, 1H), 4.02-4.18 (m, 2H), 1.40 (s, 9H).

Step 2. To a stirred solution of 2-aminopyridine-3-carbaldehyde (0,430 g. 3.53 mmol) and tert-butyl (4-((3-nitropyridin-2-yl)amino)benzyl)carbamate CK₂ (0.900 g. 2.61 mmol) in DMSO (15.0 mL) and methanol (3.00 mL), Na₂S₂O₄ (1.36 g, 7.84 mmol) was added and the mixture was stirred at 100° C. under N₂ for 12 hrs. Water (200 mL) was added to the reaction mixture, and the mixture extracted with EtOAc (300 mL×3). The organic layer was washed with brine, then dried over Na₂SO₄ and concentrated in vacuum. The crude was purified by flash column (0-80% of EtOAc in PE) to give tert-butyl (4-(2-(2-aminopyridin-3-yl)-3H-imidazo[4,5-b]pyridin-3-yl)benzyl)carbamate CK3 (240 mg, 0.576 mmol, 22.1% yield) as an orange solid. ¹H NMR (400 MHz, DMSO-d₆) δ_{H}=8.31 (dd, J=4.8, 1.2 Hz, 1H), 8.20 (dd, J=8.0, 1.2 Hz, 1H), 7.99 (dd, J=4.8, 2.0 Hz, 1H), 7.49 (t, J=6.0 Hz, 1H), 7.42-7.34 (m, 5H), 7.20 (dd, J=7.6, 1.6 Hz, 1H), 6.99 (s, 2H), 6.35-6.29 (m, 1H), 4.21 (d, J=6.0 Hz, 2H), 1.41 (s, 9H).

Step 3. To a solution of tert-butyl (4-(2-(2-aminopyridin-3-yl)-3H-imidazo[4,5-b]pyridin-3-yl)benzyl)carbamate CK3 (300 mg, 0.720 mmol) in 1,4-dioxane (6.00 mL), HCl/1,4-dioxane (15.0 mL) was added. After stirring at 25° C. for 16 hrs, the reaction mixture was concentrated in vacuum to give 3-[3-[4-(aminomethyl)phenyl]imidazo[4,5-b]pyridin-2-yl]pyridin-2-amine hydrochloride CK4 (300 mg, crude) as an orange solid. ¹H NMR (400 MHz, DMSO-d₆) δ_{H}=8.74-8.55 (m, 3H), 8.51-8.38 (m, 2H), 8.30 (dd, J=8.0, 1.2 Hz, 1H), 8.16 (dd, J=6.0, 1.2 Hz, 1H), 7.94 (dd, J=7.6, 1.6 Hz, 1H) 7.68 (d, J=8.4 Hz, 2H), 7.57 (d, J=8.4 Hz, 2H), 7.48 (dd, J=8.0, 4.8 Hz, 1H), 6.95-6.85 (m, 1H), 4.15-4.04 (m, 2H).

Step 4. To a solution of 3-(2-methoxy-2-oxo-ethyl)benzoic acid (100 mg, 0.515 mmol) in DMF (2.00 mL), HATU (235 mg, 0.618 mmol) and DIEA (0.270 mL, 1.54 mmol) were added at 25° C. and the mixture stirred for 15 mins. A solution of Mar. 13, 2014-(aminomethyl)phenyl]imidazo[4,5-b]pyridin-2-yl]pyridin-2-amine CK4 (163 mg, 0.515 mmol) in DMF (2.00 mL) was then added, and the reaction was stirred at 25° C. for 3 hrs. Water (10 mL×3) was added to the reaction mixture, and the whole extracted with EtOAc (20 mL×3). The organic layer was dried over Na₂SO₄, filtered and the filtrate was concentrated in vacuum. The crude material was purified by Welch Xtimate C18 150*30 mm*5 μm water (FA)-ACN to give methyl 2-(3-((4-(2-(2-aminopyridin-3-yl)-3H-imidazo[4,5-b]pyridin-3-yl)benzyl)carbamoyl)phenyl)acetate 100 (39.0 mg, 0.0752 mmol, 14.6% yield) as a yellow solid. ¹H NMR (400 MHz, DMSO-d₆) δ_{H}=8.38 (dd, J=4.8, 1.6 Hz, 1H), 8.25 (dd, J=8.0, 1.2 Hz, 1H), 8.03 (dd, J=6.0, 1.6 Hz, 1H), 7.84-7.73 (m, 3H), 7.49-7.39 (m, 7H), 6.78 (dd, J=7.2, 6.0 Hz, 1H), 4.54 (s, 2H), 3.75 (s, 2H), 3.61 (s, 3H). HPLC Rₜ=2.148 min in 8 min chromatography, purity 94.8%. LCMS Rₜ=2.185 min in 4 min chromatography, purity 95.8%, MS ESI calcd. For 492.19 [M+H]⁺ 493.19, found 493.1.

### Example 101. Synthesis of 2-(3-((4-(2-(2-aminopyridin-3-yl)-3H-imidazo[4,5-b]pyridin-3-yl)benzyl)carbamoyl)phenyl) acetic acid (Compound 101)

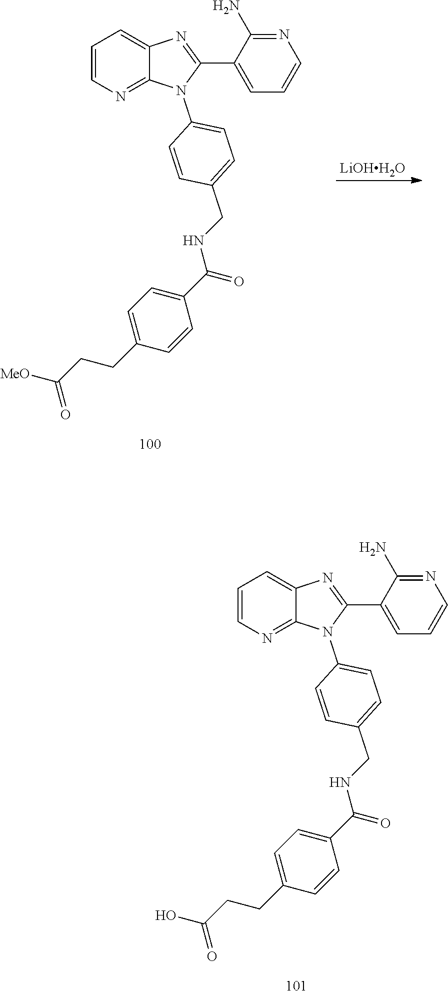 [SMILES: COC(=O)CCc1ccc(C(=O)NCc2ccc(-n3c(-c4cccnc4N)nc4cccnc43)cc2)cc1 ; "LiOH·H2O" ; "100" ; Nc1ncccc1-c1nc2cccnc2n1-c1ccc(CNC(=O)c2ccc(CCC(=O)O)cc2)cc1 ; "101"]

Step 1. To a solution of methyl 2-(3-((4-(2-(2-aminopyridin-3-yl)-3H-imidazo[4,5-b]pyridin-3-yl)benzyl)carbamoyl)phenyl)acetate 100 (90.0 mg, 0.183 mmol) in THF (1.00 mL) and water (1.00 mL), LiOH·H₂O (15.0 mg, 0.365 mmol) was added and the reaction mixture was stirred at 25° C. for 4 hrs. The reaction mixture was purified by HPLC Welch Xtimate C18 150*30 mm*5 μm water (FA)-ACN to give 2-(3-((4-(2-(2-aminopyridin-3-yl)-3H-imidazo[4,5-b]pyridin-3-yl)benzyl)carbamoyl)phenyl) acetic acid 101 (26.0 mg, 0.0517 mmol, 28.3% yield) as a white solid. ¹H NMR (400 MHz, DMSO-d₆) δ_{H}=12.61-12.13 (m, 1H), 9.13 (t, J=5.6 Hz, 1H), 8.31 (d, J=4.0 Hz, 1H), 8.24-8.18 (m, 1H), 8.13 (s, 1H), 8.02-7.97 (m, 1H), 7.85-7.76 (m, 2H), 7.36-7.50 (m, 7H), 7.30 (dd, J=7.2, 1.6 Hz, 1H), 7.11 (s, 1H), 6.45 (dd, J=7.2, 4.8 Hz, 1H), 4.56 (d, J=6.0 Hz, 2H), 3.65 (s, 2H). HPLC Rₜ=3.660 min in 8 min chromatography, purity 94.1%. LCMS Rₜ=2.204 min in 4 min chromatography, purity 95.8%, MS ESI calcd. For 478.18 [M+Na]⁺ 479.18, found 479.2.

### Example 102. Synthesis of 4-((4-(2-(2-aminopyridin-3-yl)-6-methoxy-1H-benzo[d]imidazol-1-yl)benzyl)carbamoyl)-3-methylbenzoic acid (Compound 102)

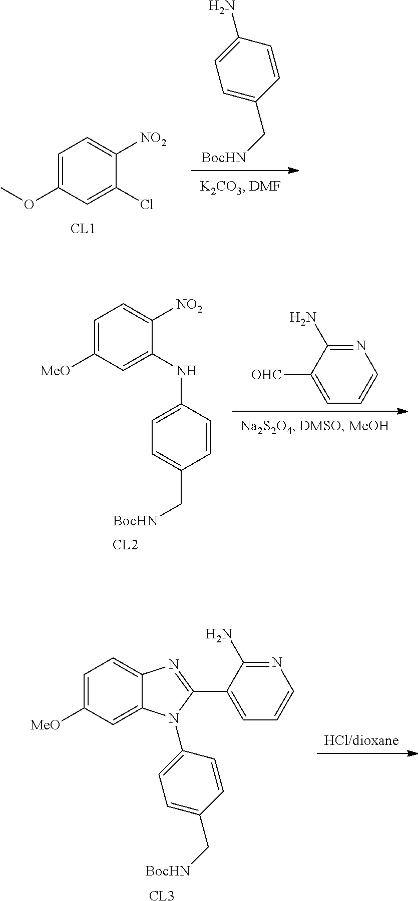

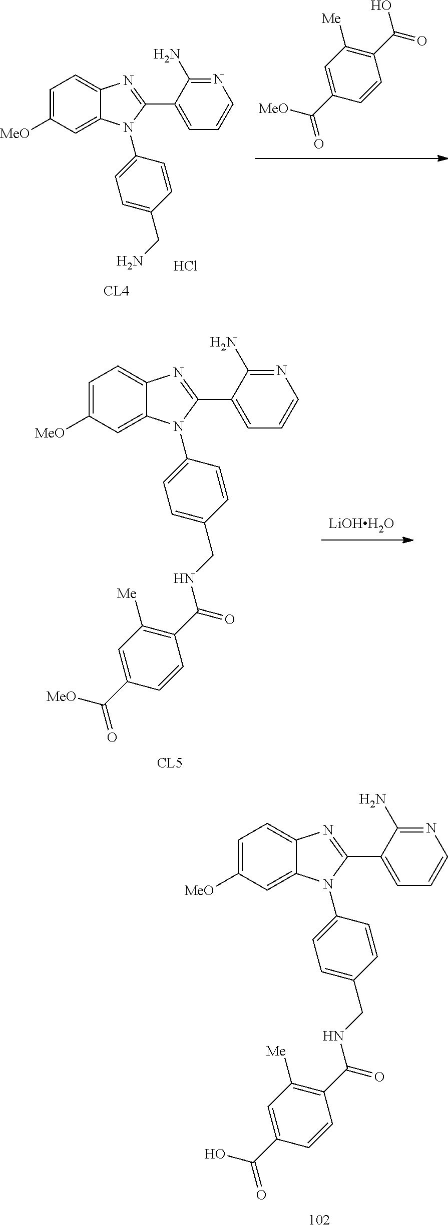

Step 1. To a solution of 2-fluoro-4-methoxy-1-nitro-benzene CL1 (2.00 g, 11.7 mmol) and tert-butyl N-[(4-aminophenyl)methyl]carbamate (2.73 g, 12.3 mmol) in DMF (20.0 mL), K₂CO₃ (3.20 g, 23.4 mmol) was added and the reaction mixture was stirred at 90° C. for 5 hrs. The reaction mixture was diluted with EtOAc (100 mL), washed with water (100 mL) and brine (100 ml×2), dried over anhydrous sodium sulfate, filtered and the filtrate was concentrated and purified by flash chromatography on silica gel (35% of EtOAc in PE) to afford tert-butyl N-[[4-(5-methoxy-2-nitro-anilino)phenyl]methyl]carbamate CL2 (1.10 g, 2.90 mmol, 25.2% yield) as a yellow oil. ¹H NMR (400 MHz, DMSO-d₆) δ=9.63 (s, 1H), 8.12 (d, J=9.6 Hz, 1H), 7.41 (t, J=6.0 Hz, 1H), 7.37-7.24 (m, 4H), 6.56-6.42 (m, 2H), 4.14 (d, J=6.0 Hz, 2H), 3.72 (s, 3H), 1.40 (s, 9H).

Step 2. To a stirred solution of 2-aminopyridine-3-carbaldehyde (442 mg, 3.62 mmol) and tert-butyl N-[[4-(5-methoxy-2-nitro-anilino)phenyl]methyl]carbamate CL2 (1.00 g. 2.68 mmol) in DMSO (20.0 mL) and methanol (5.00 mL), Naz$204 (1.40 g, 8.03 mmol) was added and the reaction was stirred at 90° C. under N₂ for 16 hrs. The reaction was diluted with EtOAc (100 mL), washed with water (100 mL) and brine (200 mL), dried over anhydrous sodium sulfate, filtered, and the filtrate was concentrated and purified by flash chromatography on silica gel (95% of EtOAc in PE) to afford tert-butyl N-[[4-[2-(2-amino-3-pyridyl)-6-methoxy-benzimidazol-1-yl]phenyl]methyl]carbamate CL3 (500 mg, 1.12 mmol, 41.9% yield) as a yellow oil. ¹H NMR (400 MHz, DMSO-d₆) δ=7.93 (dd, J=2.0, 4.8 Hz, 1H), 7.68 (d, J=8.8 Hz, 1H), 7.55-7.35 (m, 5H), 7.09-6.98 (m, 2H), 6.94 (dd, J=2.4, 8.8 Hz, 1H), 6.60 (d, J=2.0 Hz, 1H), 6.33 (dd, J=4.8, 7.6 Hz, 1H), 4.24 (d, J=6.0 Hz, 2H), 3.72 (s, 3H), 1.41 (s, 9H).

Step 3. To a stirred solution of tert-butyl N-[[4-[2-(2-amino-3-pyridyl)-6-methoxy-benzimidazol-1-yl]phenyl]methyl]carbamate CL3 (150 mg, 0.340 mmol) in 1,4-dioxane (2.00 mL), HCl/dioxane (2.00 mL, 4 M) was added and the reaction was stirred at 25° C. under N₂ for 16 hrs. The reaction was then concentrated to afford 3-[1-[4-(aminomethyl)phenyl]-6-methoxy-benzimidazol-2-yl]pyridin-2-amine hydrochloride CL4 (100 mg, 0.290 mmol, 85.9% yield) as a yellow solid. ¹H NMR (400 MHz, DMSO-d₆) δ=8.50 (d, J=2.0 Hz, 4H), 8.08 (dd, J=1.6, 6.0 Hz, 1H), 7.78 (d, J=8.8 Hz, 1H), 7.74-7.65 (m, 3H), 7.59 (d, J=8.4 Hz, 2H), 7.05 (dd, J=2.4, 8.8 Hz, 1H), 6.79 (dd, J=6.0, 7.6 Hz, 1H), 6.65 (d, J=2.4 Hz, 1H), 4.17-4.09 (m, 2H), 3.75 (s, 3H).

Step 4. To a stirred solution of 3-[1-[4-(aminomethyl)phenyl]-6-methoxy-benzimidazol-2-yl]pyridin-2-amine hydrochloride CL4 (100 mg, 0.290 mmol) and 4-methoxycarbonyl-2-methyl-benzoic acid (56.0 mg, 0.290 mmol) in pyridine (2.00 mL), EDCI (61.0 mg, 0.320 mmol) was added and the reaction mixture stirred at 25° C. under N₂ for 16 hrs. The reaction mixture was diluted with EtOAc (10 mL), washed with 1 M HCl (10 mL×2), dried over anhydrous sodium sulfate, filtered and the filtrate was concentrated. The crude was purified by flash chromatography on silica gel (100% of EtOAc in PE) to afford methyl 4-((4-(2-(2-aminopyridin-3-yl)-6-methoxy-1H-benzo[d]imidazol-1-yl)benzyl)carbamoyl)-3-methylbenzoate CL5 (20.0 mg, 13.2% yield) as a yellow oil. LCMS Rₜ=0.429 min in 1.0 min chromatography, purity 97.4%, MS ESI calcd, for 521.21 [M+H]⁺ 522.21, found 522.2.

Step 5. To a stirred solution of methyl 4-((4-(2-(2-aminopyridin-3-yl)-6-methoxy-1H-benzo[d]imidazol-1-yl)benzyl)carbamoyl)-3-methylbenzoate CL5 (20.0 mg, 0.0383 mmol) in THF (1.00 mL) and water (1.00 mL), LiOH·H₂O (3.10 mg, 0.0700 mmol) was added and the reaction mixture was stirred at 25° C. under N₂ for 10 hrs. The reaction mixture was adjusted to pH ~5 with 1 M HCl and concentrated to afford a residue. The crude product was purified by prep-HPLC (column: welch xtimate c18 150*30 mm*5 μm, method: water (FA)-ACN, begin B: 13, end B: 43) to afford 4-((4-(2-(2-aminopyridin-3-yl)-6-methoxy-1H-benzo[d]imidazol-1-yl)benzyl)carbamoyl)-3-methylbenzoic acid 102 (18.0 mg, 88.6% yield) as a white solid. ¹H NMR (400 MHz, DMSO-d₆) δ=13.10 (s, 1H), 9.04 (t, J=6.0 Hz, 1H), 7.94 (dd, J=1.6, 4.8 Hz, 1H), 7.88-7.76 (m, 2H), 7.69 (d, J=8.8 Hz, 1H), 7.58-7.47 (m, 3H), 7.41 (d, J=8.4 Hz, 2H), 7.14-6.99 (m, 3H), 6.95 (dd, J=2.4, 8.8 Hz, 1H), 6.61 (d, J=2.4 Hz, 1H), 6.34 (dd, J=4.8, 7.6 Hz, 1H), 4.56 (d, J=6.0 Hz, 2H), 3.73 (s, 3H), 2.37 (s, 3H). HPLC Rₜ=4.299 min in 8 min chromatography, purity 97.4%. LCMS Rₜ=2.300 min in 4 min chromatography, purity 98.0%, MS ESI calcd, for 507.19 [M+H]⁺ 508.19, found 508.0.

### Example 103. Synthesis of 3-((4-(2-(2-aminopyridin-3-yl)-6-methoxy-1H-benzo[d]imidazol-1-yl)benzyl)carbamoyl)-2-fluorobenzoic acid (Compound 103)

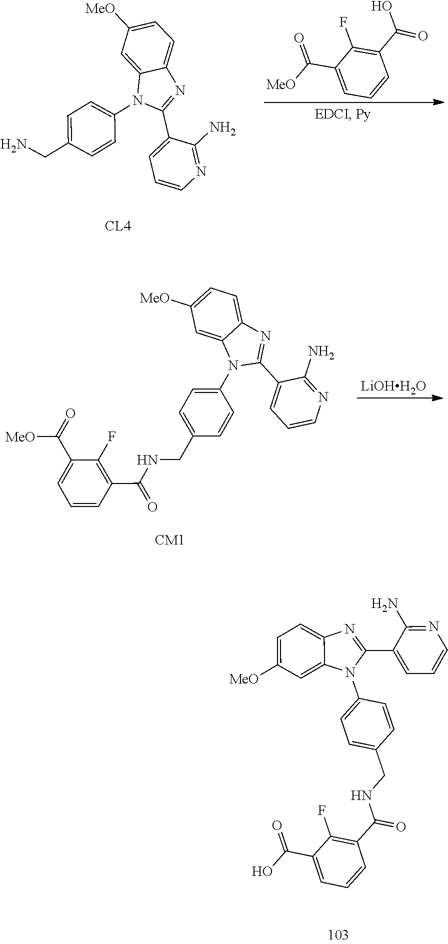

Step 1. To a stirred solution of 2-fluoro-3-methoxycarbonyl-benzoic acid (57.0 mg, 0.290 mmol) and 3-[1-[4-(aminomethyl)phenyl]-6-methoxy-benzimidazol-2-yl]pyridin-2-amine CL4 (100 mg, 0.290 mmol) in pyridine (2.00 mL), EDCI (61.0 mg, 0.320 mmol) was added and the reaction mixture was stirred at 25° C. under N₂ for 16 hrs. The reaction mixture was diluted with EtOAc (20 mL) and washed with 1N HCl (20 mL×3), brine (20 mL×3), dried over anhydrous sodium sulfate, filtered, and the filtrate was concentrated and purified by flash chromatography on silica gel (100% of EtOAc in PE) to afford methyl 3-((4-(2-(2-aminopyridin-3-yl)-6-methoxy-1H-benzo[d]imidazol-1-yl)benzyl)carbamoyl)-2-fluorobenzoate CM1 (20.0 mg, 0.0380 mmol, 13.1% yield) as a yellow oil. LCMS Rₜ=0.42 min in 1.0 min chromatography, purity 93.6%, MS ESI calcd, for 525.18 [M+H]⁺ 526.18, found 526.2.

Step 2. To a stirred solution of methyl 3-((4-(2-(2-aminopyridin-3-yl)-6-methoxy-1H-benzo[d]imidazol-1-yl)benzyl)carbamoyl)-2-fluorobenzoate CM1 (20.0 mg, 0.0380 mmol) in THF (1.00 mL) and water (1.00 mL), LiOH·H₂O (3.00 mg, 0.0700 mmol) was added and the reaction mixture was stirred at 25° C. under N₂ for 16 hrs. The reaction mixture was adjusted to pH ~4 with 1N HCl and concentrated to afford a residue. The crude product was purified by prep-HPLC (column: welch xtimate c18 150*30 mm*5 μm, method: water (FA)-ACN, begin B: 10, end B 40) to afford 3-((4-(2-(2-aminopyridin-3-yl)-6-methoxy-1H-benzo[d]imidazol-1-yl)benzyl)carbamoyl)-2-fluorobenzoic acid 103 (8.40 mg, 0.0160 mmol, 43.2% yield) as a yellow solid. ¹H NMR (400 MHz, DMSO-d₆) δ_{H}=13.40 (s, 1H), 9.13 (s, 1H), 8.04-7.90 (m, 2H), 7.84 (s, 1H), 7.69 (d, J=8.8 Hz, 1H), 7.52 (d, J=8.4 Hz, 2H), 7.46-7.34 (m, 3H), 7.19-7.01 (m, 3H), 6.95 (dd, J=2.4, 8.8 Hz, 1H), 6.62 (d, J=2.4 Hz, 1H), 6.38 (dd, J=4.8, 7.6 Hz, 1H), 4.59 (d, J=6.0 Hz, 2H), 3.73 (s, 3H). HPLC Rₜ=4.239 min in 8 min chromatography, purity 98.8%. LCMS Rₜ=2.251 min in 4 min chromatography, purity 98.9%, MS ESI calcd, for 511.17 [M+H]+512.17, found 512.1.

### Example 104. Synthesis of 2-(3-((4-(2-(2-aminopyridin-3-yl)-6-methoxy-1H-benzo[d]imidazol-1-yl)benzyl)carbamoyl)phenyl) acetic acid (Compound 104)

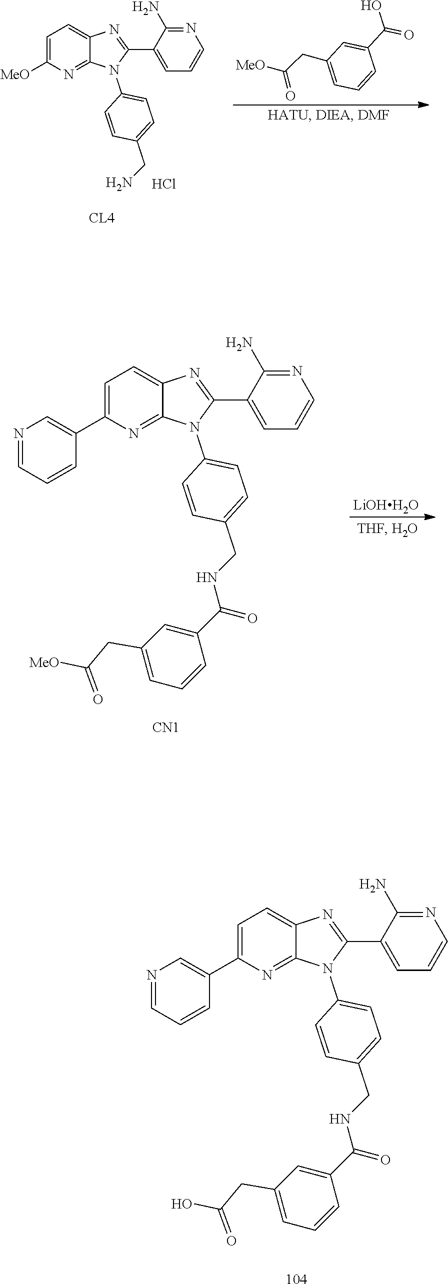

Step 1. To a stirred solution of 3-(2-methoxy-2-oxo-ethyl)benzoic acid (51.0 mg, 0.260 mmol) in DMF (2.00 mL), DIEA (0.140 mL, 0.780 mmol) and HATU (109 mg, 0.290 mmol) were added and the mixture was stirred at 25° C., under N₂, for 20 mins. A solution of 3-[1-[4-(aminomethyl)phenyl]-6-methoxy-benzimidazol-2-yl]pyridin-2-amine hydrochloride CL4 (90.0 mg, 0.260 mmol) in DMF (1.00 mL) was then added to the reaction, and the resulting mixture was stirred at 25° C. under N₂ for 16 hrs. The reaction was diluted with EtOAc (20 mL), washed with water (20 mL) and brine (20 mL×2), dried over anhydrous sodium sulfate, filtered, and the filtrate was concentrated and purified by flash chromatography on silica gel (100% of EtOAc in PE) to afford methyl 2-(3-((4-(2-(2-aminopyridin-3-yl)-6-methoxy-1H-benzo[d]imidazol-1-yl)benzyl)carbamoyl)phenyl)acetate CN1 (120 mg, 0.230 mmol, 88.2% yield) as a yellow solid. ¹H NMR (400 MHz, CDCl₃) δ=8.00 (dd, J=2.0, 4.8 Hz, 1H), 7.82-7.66 (m, 3H), 7.57-7.40 (m, 4H), 7.30 (d, J=8.4 Hz, 2H), 7.07-6.90 (m, 2H), 6.69-6.48 (m, 4H), 6.35 (dd, J=4.8, 7.6 Hz, 1H), 4.78 (d, J=6.0 Hz, 2H), 3.80 (s, 3H), 3.74-3.68 (m, 5H).

Step 2. To a stirred solution of methyl 2-(3-((4-(2-(2-aminopyridin-3-yl)-6-methoxy-1H-benzo[d]imidazol-1-yl)benzyl)carbamoyl)phenyl)acetate CN1 (120 mg, 0.230 mmol) in THE (1.00 mL) and water (1.00 mL), LiOH·H₂O (18.0 mg, 0.460 mmol) was added and the reaction was stirred at 25° C. under N₂ for 16 hrs. The reaction mixture was adjusted to pH ~5 with aq. HCl and concentrated to afford a residue. The crude product was diluted with DMSO (3 mL) and purified by prep-HPLC (column: Welch Xtimate C18 150*30 mm*5 μm, method: water (FA)-ACN, begin B: 12, end B: 42) to afford 2-(3-((4-(2-(2-aminopyridin-3-yl)-6-methoxy-1H-benzo[d]imidazol-1-yl)benzyl)carbamoyl)phenyl) acetic acid 104 (63.0 mg, 0.120 mmol, 53.0% yield) as a yellow solid. ¹H NMR (400 MHz, DMSO-d₆) δ=12.40 (s, 1H), 9.14 (t, J=6.0 Hz, 1H), 7.93 (J=1.2, 4.8 Hz, 1H), 7.87-7.76 (m, 2H), 7.68 (d, J=8.8 Hz, 1H), 7.54-7.30 (m, 6H), 7.14-6.85 (m, 4H), 6.61 (d, J=2.0 Hz, 1H), 6.35 (dd, J=4.8, 7.6 Hz, 1H), 4.60 (d, J=6.0 Hz, 2H), 3.72 (s, 3H), 3.65 (s, 2H). HPLC Rₜ=4.257 min in 8 min chromatography, purity 98.9%. LCMS Rₜ=2.268 min in 4 min chromatography, purity 98.8%, MS ESI calcd, for 507.19 [M+H]+508.19, found 508.1.

### Example 105. Synthesis of 4-((4-(2-(2-aminopyridin-3-yl)-6-(trifluoromethyl)-1H-benzo[d]imidazol-1-yl)benzyl)carbamoyl)-3-methylbenzoic acid (Compound 105)

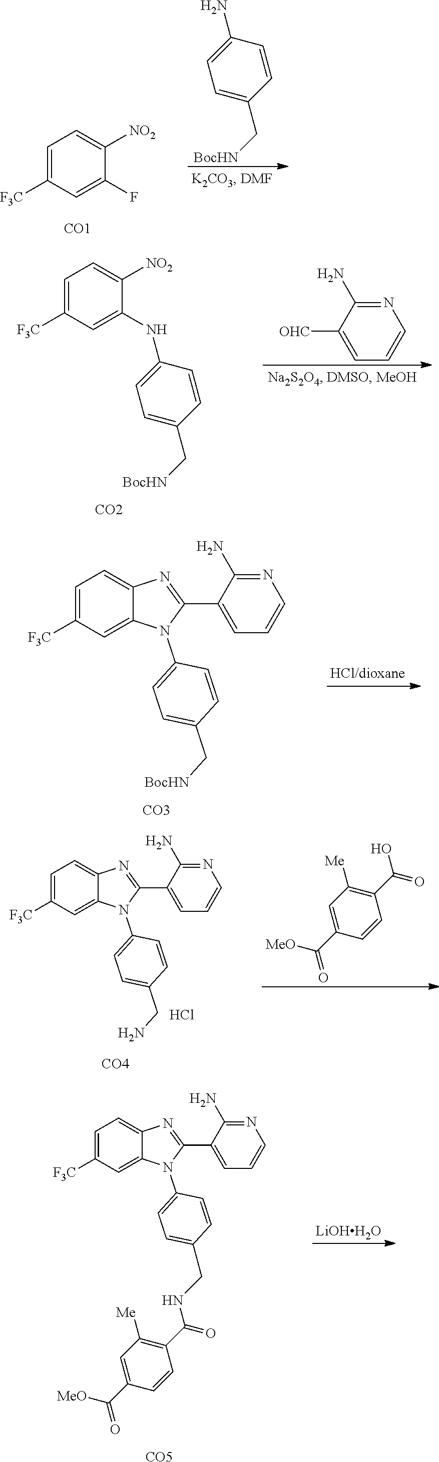

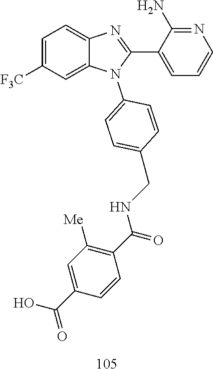 [SMILES: Cc1cc(C(=O)O)ccc1C(=O)NCc1ccc(-n2c(-c3cccnc3N)nc3ccc(C(F)(F)F)cc32)cc1 ; "105"]

Step 1. To a solution of 2-fluoro-1-nitro-4-(trifluoromethyl)benzene CO1 (2.00 g, 9.56 mmol) and tert-butyl N-[(4-aminophenyl)methyl]carbamate (2.20 g, 10.0 mmol) in DMF (20.0 mL), K₂CO₃ (2.60 g. 19.1 mmol) was added and the reaction mixture was stirred at 90° C. for 5 hrs. The reaction mixture was diluted with EtOAc (100 mL), washed with water (100 mL) and brine (150 mL×2), dried over anhydrous sodium sulfate, filtered, and the filtrate was concentrated and purified by flash chromatography on silica gel (30% of EtOAc in PE) to afford tert-butyl N-[[4-[2-nitro-5-(trifluoromethyl) anilino]phenyl]methyl]carbamate CO2 (2.0 g, 4.86 mmol, 50.8% yield) as a yellow oil. ¹H NMR (400 MHz, DMSO-d₆) δ=9.53 (s, 1H), 8.30 (d, J=8.4 Hz, 1H), 8.16 (d, J=8.4 Hz, 1H), 7.82 (d, J=8.4 Hz, 1H), 7.60 (s, 1H), 7.47-7.22 (m, 1H), 7.17-7.06 (m, 2H), 6.85 (d, J=8.4 Hz, 1H), 4.15 (d, J=6.0 Hz, 2H), 1.40 (s, 9H).

Step 2. To a stirred solution of 2-aminopyridine-3-carbaldehyde (802 mg, 6.56 mmol) and tert-butyl N-[4-[2-nitro-5-(trifluoromethyl) anilino]phenyl methyl]carbamate CO2 (2.00 g, 4.86 mmol) in DMSO (20.0 mL) and methanol (2.00 mL), Na₂S₂O₄ (2.50 g, 14.6 mmol) was added, and the reaction was stirred at 90° C. under N₂ for 16 hrs. The reaction was diluted with EtOAc (10 mL), washed with water (10 mL) and brine (30 mL), dried over anhydrous sodium sulfate, filtered and the filtrate was concentrated and purified by flash chromatography on silica gel (95% EtOAc in PE) to afford tert-butyl N-[[4-[2-(2-amino-3-pyridyl)-6-(trifluoromethyl)benzimidazol-1-yl]phenyl]methyl]carbamate CO₃ (900 mg, 1.86 mmol, 38.3% yield) as a yellow oil. ¹H NMR (400 MHz, DMSO-d₆) δ=8.04-7.96 (m, 2H), 7.69-7.63 (m, 1H), 7.55-7.35 (m, 6H), 7.23-7.15 (m, 1H), 7.97 (s, 2H), 6.42-6.36 (m, 1H), 4.24 (d, J=6.0 Hz, 2H), 1.41 (s, 9H).

Step 3. To a stirred solution of tert-butyl N-[[4-[2-(2-amino-3-pyridyl)-6-(trifluoromethyl)benzimidazol-1-yl]phenyl methyl]carbamate CO₃ (150 mg, 0.310 mmol) in 1,4-dioxane (2.00 mL), HCl/dioxane (2.00 mL, 4 M) was added and the reaction was stirred at 25° C., under N₂, for 16 hrs. The reaction was concentrated to afford 3-[1-[4-(aminomethyl)phenyl]-6-(trifluoromethyl)benzimidazol-2-yl]pyridin-2-amine hydrochloride CO4 (100 mg, 0.260 mmol, 84.1% yield) as a yellow solid. ¹H NMR (400 MHz, DMSO-d₆) δ=8.55-8.30 (m, 3H), 8.20-8.04 (m, 2H), 7.84 (d, J=7.6 Hz, 1H), 7.77-7.69 (m, 3H), 7.67-7.59 (m, 2H), 7.45 (s, 1H), 6.84 (t, J=6.8 Hz, 1H), 4.18-4.10 (m, 2H).

Step 4. To a stirred solution of 3-[1-[4-(aminomethyl)phenyl]-6-(trifluoromethyl)benzimidazol-2-yl]pyridin-2-amine hydrochloride CO₄ (100 mg, 0.260 mmol) and 4-methoxycarbonyl-2-methyl-benzoic acid (51.0 mg, 0.260 mmol) in pyridine (2.00 mL), EDCI (55.0 mg, 0.290 mmol) was added and the reaction mixture was stirred at 25° C. under N₂ for 16 hrs. The reaction mixture was diluted with EtOAc (10 mL), washed with 1 M HCl (10 mL×2), dried over anhydrous sodium sulfate, filtered, and the filtrate was concentrated. The crude was purified by flash chromatography on silica gel (100% of EtOAc in PE) to afford methyl 4-((4-(2-(2-aminopyridin-3-yl)-6-(trifluoromethyl)-1H-benzo[d]imidazol-1-yl)benzyl)carbamoyl)-3-methylbenzoate CO5 (70.0 mg, 47.9% yield) as a yellow oil. LCMS Rₜ=0.50 min in 1.0 min chromatography, purity 95.7%, MS ESI calcd. for 559.18 [M+H]⁺ 560.18, found 560.1.

Step 5. To a stirred solution of methyl 4-((4-(2-(2-aminopyridin-3-yl)-6-(trifluoromethyl)-1H-benzo[d]imidazol-1-yl)benzyl)carbamoyl)-3-methylbenzoate CO5 (70.0 mg, 0.130 mmol) in THF (1.00 mL) and water (1.00 mL), LiOH·H₂O (10.0 mg, 0.250 mmol) was added and the reaction mixture was stirred at 25° C. under N₂ for 16 hrs. The reaction mixture was adjusted to pH ~5 with 1 M HCl and concentrated. The crude product was purified by prep-HPLC (Column Welch Xtimate C18 150*30 mm*5 μm, Condition water (FA)-ACN Begin B: 20, End B: 50) to afford 4-((4-(2-(2-aminopyridin-3-yl)-6-(trifluoromethyl)-1H-benzo[d]imidazol-1-yl)benzyl)carbamoyl)-3-methylbenzoic acid 105 (32.0 mg, 46.9% yield) as a white solid. ¹H NMR (400 MHz, DMSO-d₆) δ=12.99 (s, 1H), 9.03 (s, 1H), 8.10-7.93 (m, 2H), 7.88-7.76 (m, 2H), 7.74-7.62 (m, 1H), 7.61-7.45 (m, 5H), 7.39 (s, 1H), 7.21 (dd, J=1.6, 7.6 Hz, 1H), 6.98 (s, 2H), 6.40 (dd, J=4.8, 7.6 Hz, 1H), 4.56 (d, J=6.0 Hz, 2H), 2.37 (s, 3H). 1° F. NMR (400 MHz, DMSO-d₆) δ=−59.121. HPLC Rₜ=4.941 min in 8 min chromatography, purity 98.9%. LCMS Rₜ=2.583 min in 4 min chromatography, purity 100%, MS ESI calcd, for 545.17 [M+H]⁺ 546.17, found 546.1.

### Example 106. Synthesis of 3-((4-(2-(2-aminopyridin-3-yl)-6-(trifluoromethyl)-1H-benzo[d]imidazol-1-yl)benzyl)carbamoyl)-2-fluorobenzoic acid (Compound 106)

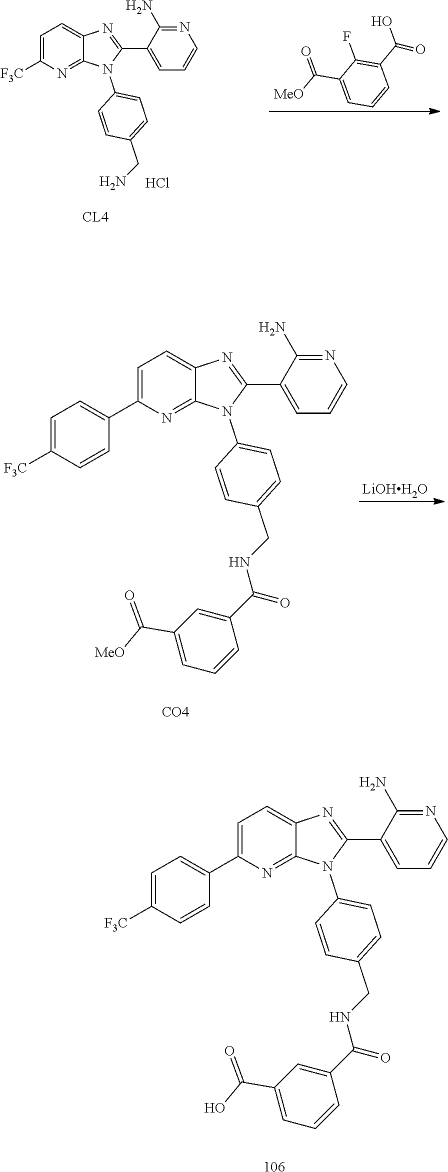

Step 1. To a stirred solution of 2-fluoro-3-methoxycarbonyl-benzoic acid (60.0 mg, 0.310 mmol) and 3-[1-[4-(aminomethyl)phenyl]-6-(trifluoromethyl)benzimidazol-2-yl]pyridin-2-amine hydrochloride CO₄ (100 mg, 0.260 mmol) in pyridine (2.00 mL), EDCI (55.0 mg, 0.290 mmol) was added and the reaction mixture was stirred at 25° C. under N₂ for 16 hrs. The reaction mixture was diluted with EtOAc (20 mL), washed with 1N HCl (20 mL×3), brine (20 mL×3), dried over anhydrous sodium sulfate, filtered and the filtrate was concentrated and purified by flash chromatography on silica gel (100% of EtOAc in PE) to afford methyl 3-((4-(2-(2-aminopyridin-3-yl)-6-(trifluoromethyl)-1H-benzo[d]imidazol-1-yl)benzyl)carbamoyl)-2-fluorobenzoate CP1 (60.0 mg, 0.110 mmol, 40.8% yield) as a yellow oil. ¹H NMR (400 MHz, DMSO-d₆) δ_{H}=8.35 (d, J=1.2 Hz, 1H), 8.18-7.98 (m, 2H), 7.91 (d, J=8.4 Hz, 1H), 7.65-7.53 (m, 3H), 7.46 (s, 1H), 7.42-7.29 (m, 3H), 7.06 (dd, J=2.0, 7.6 Hz, 1H), 6.39 (dd, J=4.8, 7.6 Hz, 1H), 4.82 (d, J=6.0 Hz, 2H), 3.96 (s, 3H).

Step 2. To a stirred solution of methyl 3-((4-(2-(2-aminopyridin-3-yl)-6-(trifluoromethyl)-1H-benzo[d]imidazol-1-yl)benzyl)carbamoyl)-2-fluorobenzoate CP1 (60.0 mg. 0.110 mmol) in THF (1.00 mL) and water (1.00 mL), LiOH·H₂O (8.70 mg, 0.220 mmol) was added and the reaction mixture was stirred at 25° C. under N₂ for 16 hrs. The mixture was adjusted with 1N HCl to a pH ~5 and concentrated to afford a residue. The crude product was purified by prep-HPLC (Column Welch Xtimate C18 150*30 mm*5 μm Condition water (FA)-ACN Begin B 18 End B 48) to afford 3-((4-(2-(2-aminopyridin-3-yl)-6-(trifluoromethyl)-1H-benzo[d]imidazol-1-yl)benzyl)carbamoyl)-2-fluorobenzoic acid 106 (29.0 mg, 0.0520 mmol, 48.0% yield) as a white solid. ¹H NMR (400 MHz, DMSO-d₆) δ_{H}=13.42 (s, 1H), 9.12 (t, J=6.0 Hz, 1H), 8.08-7.91 (m, 3H), 7.89-7.79 (m, 1H), 7.66 (dd, J=1.6, 8.4 Hz, 1H), 7.59-7.45 (m, 4H), 7.44-7.31 (m, 2H), 7.22 (dd, J=1.6, 7.6 Hz, 1H), 6.96 (s, 2H), 6.42 (dd, J=4.8, 7.6 Hz, 1H), 4.60 (d, J=6.0 Hz, 2H). HPLC Rₜ=4.894 min in 8 min chromatography, purity 99.6%. LCMS Rₜ=2.565 min in 4 min chromatography, purity 99.7%, MS ESI calcd. For 549.14 [M+H]⁺ 550.14, found 550.1

### Example 107. Synthesis of 2-(3-((4-(2-(2-aminopyridin-3-yl)-6-(trifluoromethyl)-1H-benzo[d]imidazol-1-yl)benzyl)carbamoyl)phenyl) acetic acid (Compound 107)

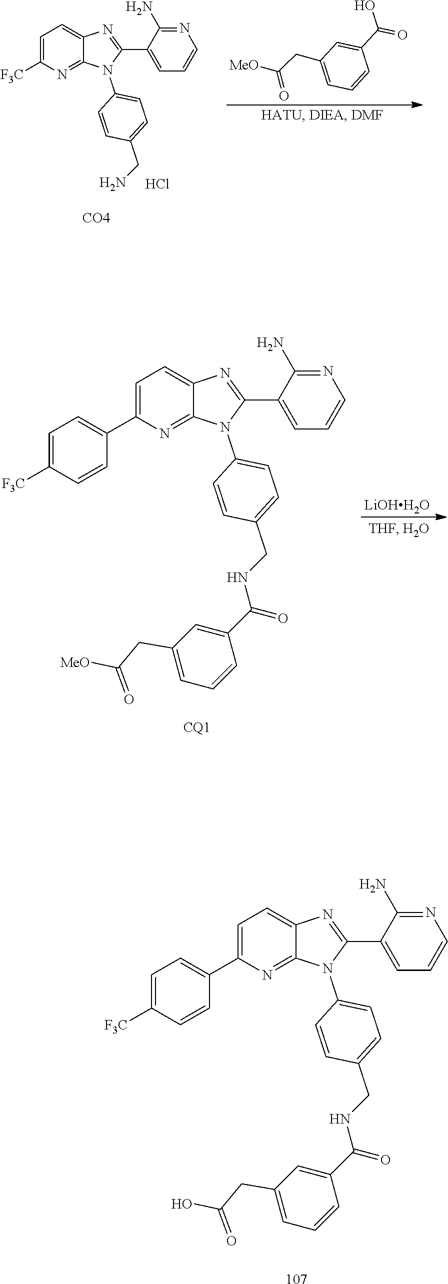

Step 1. To a stirred solution of 3-(2-methoxy-2-oxo-ethyl)benzoic acid (51.0 mg, 0.26 mmol) in DMF (2.00 mL), DIEA (0.140 mL, 0.780 mmol) and HATU (109 mg, 0.28 mmol) were added, and the mixture was stirred at 25° C. under N₂ for 20 mins. A solution of 3-[1-[4-(aminomethyl)phenyl]-6-(trifluoromethyl)benzimidazol-2-yl]pyridin-2-amine hydrochloride CO₄ (100 mg, 0.260 mmol) in DMF (1.00 mL) was then added to the reaction, and the resulting mixture was stirred at 25° C. under N₂ for 16 hrs. The reaction was diluted with EtOAc (10 mL), washed with water (10 mL) and brine (10 mL×2), dried over anhydrous sodium sulfate, filtered, and the filtrate was concentrated and purified by flash chromatography on silica gel (100% of EtOAc in PE) to afford methyl 2-(3-((4-(2-(2-aminopyridin-3-yl)-6-(trifluoromethyl)-1H-benzo[d]imidazol-1-yl)benzyl)carbamoyl)phenyl)acetate CQ1 (130 mg, 0.230 mmol, 89.1% yield) as a yellow solid. ¹H NMR (400 MHz, CDCl₃) δ=8.02 (s, 1H), 7.90 (d, J=8.4 Hz, 1H), 7.81-7.70 (m, 2H), 7.64-7.40 (m, 6H), 7.32 (d, J=8.4 Hz, 2H), 7.07 (dd, J=1.6, 8.0 Hz, 1H), 6.63 (d, J=6.0 Hz, 3H), 6.39 (dd, J=4.8, 7.6 Hz, 1H), 4.80 (d, J=6.0 Hz, 2H), 3.73-3.67 (m, 5H).

Step 2. To a stirred solution of methyl 2-(3-((4-(2-(2-aminopyridin-3-yl)-6-(trifluoromethyl)-1H-benzo[d]imidazol-1-yl)benzyl)carbamoyl)phenyl)acetate CQ1 (120 mg, 0.210 mmol) in THF (1.00 mL) and water (1.00 mL), LiOH·H₂O (17.0 mg, 0.430 mmol) was added and the reaction was stirred at 25° C. under N₂ for 16 hrs. The reaction mixture was adjusted to pH ~5 with aq. HCl solution and concentrated to afford a residue. The crude product was diluted with DMSO (3.0 mL) and purified by prep-HPLC (column: Welch Xtimate C18 150*30 mm*5 μm, method: water (FA)-ACN, begin: B 19, end B: 49) to afford 2-(3-((4-(2-(2-aminopyridin-3-yl)-6-(trifluoromethyl)-1H-benzo[d]imidazol-1-yl)benzyl)carbamoyl)phenyl) acetic acid 107 (52.0 mg, 0.0900 mmol, 43.8% yield) as a white solid. JH NMR (400 MHz, DMSO-d₆) δ=12.39 (s, 1H), 9.12 (t, J=6.0 Hz, 1H), 8.13-7.90 (m, 2H), 7.89-7.75 (m, 2H), 7.66 (d, J=8.8 Hz, 1H), 7.58-7.34 (m, 7H), 7.24 (dd, J=1.6, 7.6 Hz, 1H), 6.99 (s, 2H), 6.51-6.32 (m, 1H), 4.60 (d, J=6.0 Hz, 2H), 3.65 (s, 2H). 3F NMR (400 MHz, DMSO-d₆) δ=−59.107. HPLC Rₜ=4.905 min in 8 min chromatography, purity 99.2%. LCMS Rₜ=2.575 min in 4 min chromatography, purity 100%, MS ESI calcd, for 545.17 [M+H]⁺ 546.17, found 546.1.

### Example 108. Synthesis of 2-(3-((4-(2-(2-aminopyridin-3-yl)-1H-benzo[d]imidazol-1-yl)benzyl)carbamoyl)phenyl) acetic acid (Compound 108)

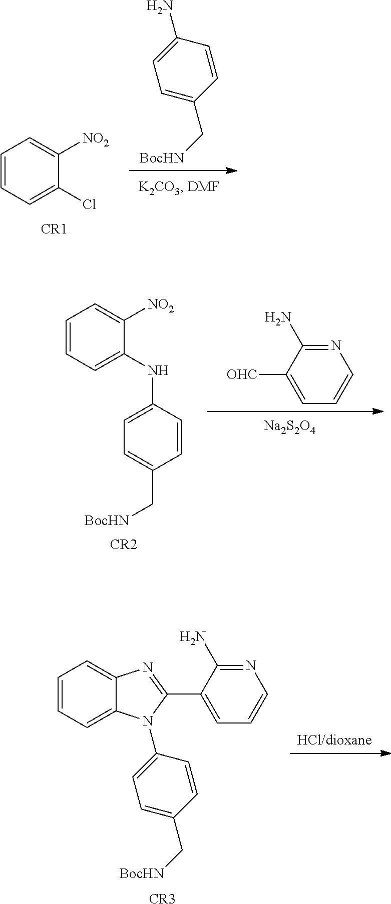

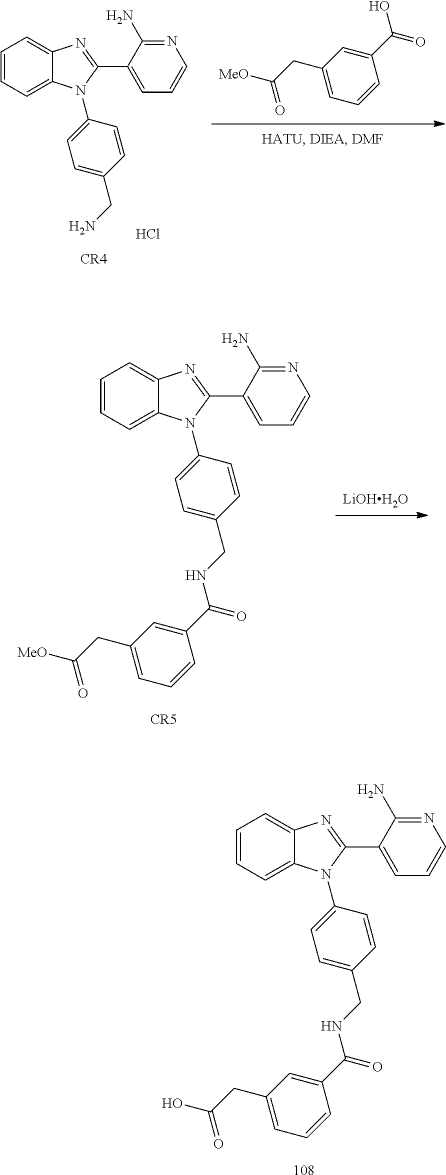

Step 1. To a stirred solution of 1-fluoro-2-nitro-benzene CR1 (0.820 mL, 7.80 mmol) and tert-butyl N-[(4-aminophenyl)methyl]carbamate (1.80 g, 8.10 mmol) in DMF (20.0 mL), K₂CO₃ (3.23 g, 23.4 mmol) was added and the reaction mixture was stirred at 80° C. under N₂ for 16 hrs. The reaction mixture was diluted with EtOAc (50 mL), washed with water (100 mL) and brine (100 mL×2), dried over anhydrous sodium sulfate, filtered, and the filtrate was concentrated and purified by flash chromatography on silica gel (25% of EtOAc in PE) to afford tert-butyl N-[[4-(2-nitroanilino)phenyl]methyl]carbamate CR2 (170 mg, 6.35% yield) as a yellow solid. ¹H NMR (400 MHz, CDCl₃) δ=9.46 (s, 1H), 8.20 (dd, J=1.6, 8.8 Hz, 1H), 7.40-7.29 (m, 3H), 7.25-7.17 (m, 3H), 7.15-6.75 (m, 1H), 4.88 (s, 1H), 4.33 (d, J=5.6 Hz, 2H), 1.48 (s, 9H).

Step 2. To a stirred solution of 2-aminopyridine-3-carbaldehyde (82.0 mg, 0.670 mmol) and tert-butyl N-[[4-(2-nitroanilino)phenyl]methyl]carbamate CR2 (170 mg, 0.490 mmol) in DMSO (3.00 mL) and methanol (0.200 mL), Na₂S₂O₄ (258 mg, 1.49 mmol) was added and the reaction mixture was stirred at 90° C. under N₂ for 10 hrs. The reaction mixture was diluted with EtOAc (15 mL), washed with water (20 mL) and brine (20 mL), dried over anhydrous sodium sulfate, filtered, and the filtrate was concentrated. The residue was purified by flash chromatography on silica gel (85% of EtOAc in PE) to afford tert-butyl N-[[4-[2-(2-amino-3-pyridyl)benzimidazol-1-yl]phenyl]methyl]carbamate CR3 (100 mg, 48.6% yield) as a yellow oil. LCMS Rₜ=0.45 min in 1.0 min chromatography, purity 97.1%, MS ESI calcd. for 415.20 [M+H]⁺ 416.20, found 416.3.

Step 3. To a stirred solution of tert-butyl N-[[4-[2-(2-amino-3-pyridyl)benzimidazol-1-yl]phenyl]methyl]carbamate CR3 (100 mg, 0.240 mmol) in 1,4-dioxane (1.00 mL), HCl/dioxane (2.00 mL, 4 M) was added and the reaction mixture was stirred at 25° C., under N₂, for 16 hrs. The reaction mixture was concentrated to afford 3-[1-[4-(aminomethyl)phenyl]benzimidazol-2-yl]pyridin-2-amine hydrochloride CR4 (80.0 mg, 75.9% yield) as a yellow solid. LCMS Rₜ=0.234 min in 1.0 min chromatography, purity 100%, MS ESI calcd, for 315.15 [M+H]⁺ 316.15, found 316.1.

Step 4. To a stirred solution of 3-(2-methoxy-2-oxo-ethyl)benzoic acid (49.0 mg, 0.250 mmol) in DMF (2.00 mL), DIEA (50.2 mL, 1.27 mmol) and HATU (106 mg, 0.280 mmol) were added and the mixture was stirred at 25° C. under N₂ for 20 mins. A solution of 3-[1-[4-(aminomethyl)phenyl]benzimidazol-2-yl]pyridin-2-amine hydrochloride CR4 (80.0 mg, 0.250 mmol) in DMF (1 mL) was then added to the mixture, and the resulting mixture was stirred at 25° C. under N₂ for 16 hrs. The reaction mixture was diluted with EtOAc (20 mL), washed with water (20 mL) and brine (20 mL×2), dried over anhydrous sodium sulfate, filtered, and the filtrate was concentrated. The crude product was purified by flash chromatography on silica gel (95% of EtOAc in PE) to afford methyl 2-(3-((4-(2-(2-aminopyridin-3-yl)-1H-benzo[d]imidazol-1-yl)benzyl)carbamoyl)phenyl)acetate CR5 (70.0 mg, 56.1% yield) as a yellow oil. LCMS Rₜ=0.42 min in 1.0 min chromatography, purity 94.1%, MS ESI calcd, for 491.20 [M+H]⁺ 492.20, found 492.2.

Step 5. To a stirred solution of methyl 2-(3-((4-(2-(2-aminopyridin-3-yl)-1H-benzo[d]imidazol-1-yl)benzyl)carbamoyl)phenyl)acetate CR5 (70.0 mg, 0.140 mmol) in THF (1.00 mL) and water (1.00 mL), LiOH·H₂O (11.0 mg, 0.290 mmol) was added and the reaction mixture was stirred at 25° C. under N₂ for 16 hrs. The reaction mixture was adjusted to pH ~5 with 1 M HCl and concentrated. The crude product was purified by prep-HPLC (column Welch Xtimate C18 150*30 mm*5 μm, condition: water (FA)-ACN, begin B: 13, end B: 43) to afford 2-(3-((4-(2-(2-aminopyridin-3-yl)-1H-benzo[d]imidazol-1-yl)benzyl)carbamoyl)phenyl) acetic acid 108 (25.0 mg, 35.8% yield) as a white solid. ¹H NMR (400 MHz, DMSO-d₆) δ=12.40 (s, 1H), 9.15 (t, J=6.0 Hz, 1H), 7.96 (dd, J=1.6, 4.8 Hz, 1H), 7.88-7.74 (m, 3H), 7.53-7.36 (m, 6H), 7.35-7.23 (m, 2H), 7.21-7.11 (m, 2H), 7.02 (s, 2H), 6.37 (dd, J=4.8, 7.6 Hz, 1H), 4.59 (d, J=6.0 Hz, 2H), 3.65 (s, 2H). HPLC Rₜ=4.203 min in 8 min chromatography, purity 97.8%. LCMS Rₜ=2.244 min in 4 min chromatography, purity 97.3%, MS ESI calcd, for 477.18 [M+H]⁺ 478.18, found 478.1.

### Synthesis of Additional Intermediates for Table 2 Compounds

### Synthesis of 4-(methoxycarbonyl)-2,5-dimethylbenzoic acid

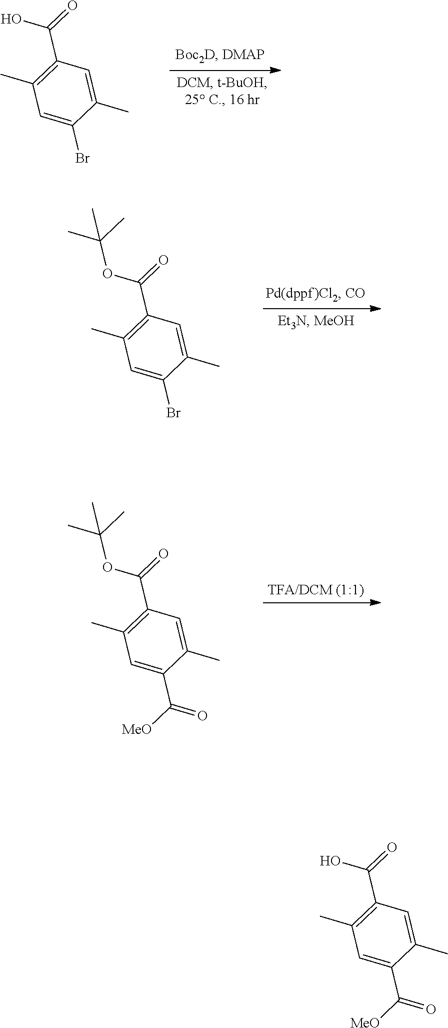

Step 1. To a solution of 4-bromo-2.5-dimethylbenzoic acid (1.00 g, 4.37 mmol) in DCM (20 mL) was added t-BuOH (10 mL, 105 mmol), Boc₂O (1.91 g, 8.73 mmol) and DMAP (0.16 g, 1.31 mmol) at 25° C. The mixture was stirred at 25° C. for 16 hrs, and then Aq. NaHCO₃ (40 mL) was added and the resulting mixture was extracted with DCM (3×10 mL). The combined organic phase was washed with brine (10 mL), water (10 mL), dried over anhydrous Na₂SO₄, filtered and concentrated. The residue was purified by flash silica gel chromatography (eluent of 0~1% EtOAc in PE) to give tert-butyl 4-bromo-2,5-dimethylbenzoate (470 mg, 38% yield) as colorless oil. ¹H NMR (400 MHz, CHLOROFORM-d) δ=7.66 (s, 1H), 7.40 (s, 1H), 2.49 (s, 3H), 2.37 (s, 3H), 1.58 (s, 9H).

Step 2. To a solution of tert-butyl 4-bromo-2,5-dimethylbenzoate (470 mg, 1.65 mmol) in methanol (10 mL) was added TEA (0.71 mL, 4.94 mmol) and Pd (dppf) C12 (60 mg, 0.0824 mmol) at 25° C. The suspension was degassed and purged with CO (50 psi) for 3 times. The mixture was stirred at 50° C. for 16 hrs then concentrated. Water (10 mL) was added and the resulting mixture was extracted with EtOAc (3×10 mL). The combined organic phase was washed with brine (5 mL), water (5 mL), dried over anhydrous Na₂SO₄, filtered and concentrated. The residue was purified by flash silica gel chromatography (eluent of 0~3% EtOAc in PE) to give 1-(tert-butyl) 4-methyl 2.5-dimethylterephthalate (320 mg, 74% yield) as colorless oil. ¹H NMR (400 MHz, CHLOROFORM-d) δ=7.76 (s, 1H), 7.66 (s, 1H), 3.92 (s, 3H), 2.58 (s, 3H), 2.55 (s, 3H), 1.62 (s, 9H).

Step 3, To a solution of 1-(tert-butyl) 4-methyl 2,5-dimethylterephthalate (320 mg, 1.21 mmol) in DCM (2 mL) was added TFA (2 mL) at 25° C. The mixture was stirred at 25° C. for 16 hrs. The reaction mixture was concentrated directly to give 4-(methoxycarbonyl)-2,5-dimethylbenzoic acid (250 mg, 99% yield) as colorless oil, which was used in the next step directly. ¹H NMR (400 MHz, CHLOROFORM-d) δ=7.91 (s, 1H), 7.78 (s, 1H), 3.92 (s, 3H), 2.61 (d, J=17.6 Hz, 6H).

### Synthesis of 5-fluoro-4-methoxycarbonyl-2-methyl-benzoic acid

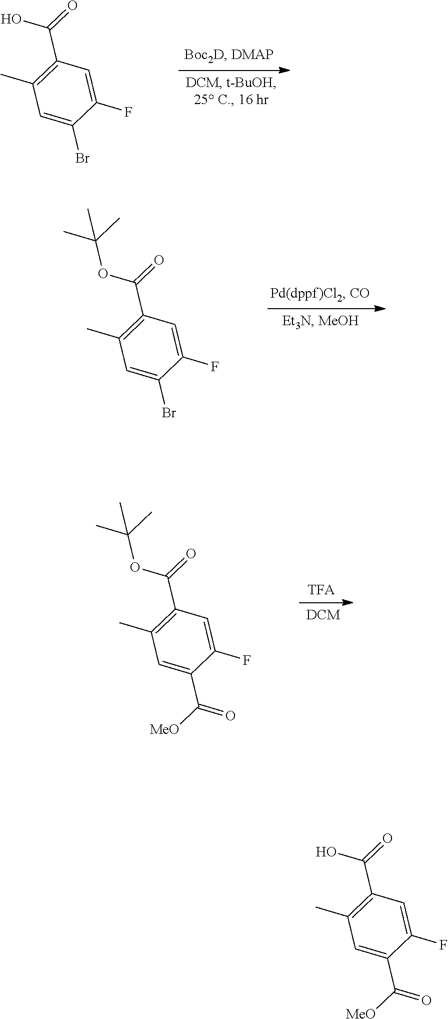 [SMILES: Cc1cc(Br)c(F)cc1C(=O)O ; Cc1cc(Br)c(F)cc1C(=O)OC(C)(C)C ; COC(=O)c1cc(C)c(C(=O)OC(C)(C)C)cc1F ; COC(=O)c1cc(C)c(C(=O)O)cc1F]

Step 1. To a solution of 4-bromo-5-fluoro-2-methyl-benzoic acid (4.00 g, 17.2 mmol) in DCM (50 mL) was added t-BuOH (40.0 mL, 418 mmol) Boc₂O) (7.49 g, 34.3 mmol) and DMAP (0.63 g, 5.15 mmol) at 25° C. The mixture was stirred at 25° C. for 16 hrs. at which point Sat aq. NaHCO₃ (50 mL) was added. The resulting mixture was extracted with DCM (3×120 mL), the combined organic layers was washed with brine (50 mL), dried over anhydrous Na₂SO₄, filtered and concentrated. The residue was purified by flash silica gel chromatography (eluent of 0~1% EtOAc in PE) to give tert-butyl 4-bromo-5-fluoro-2-methyl-benzoate (3.20 g, 65% yield) as colorless oil. ¹H NMR (400 MHz, DMSO-d₆) δ=7.65 (d, J=6.4 Hz, 1H), 7.58 (d, J=9.6 Hz, 1H), 2.43 (s, 3H), 1.52 (s, 9H).

Step 2. To a solution of tert-butyl 4-bromo-5-fluoro-2-methyl-benzoate (0.85 g, 2.94 mmol) in Methanol (20 mL) was added TEA (1.3 mL, 8.82 mmol) and Pd(dppf)Cl₂ (108 mg, 0.147 mmol) at 25° C. The suspension was degassed and purged with CO (50 Psi) for 3 times then heated to 70° C. and stirred for 16 hrs. The reaction mixture was concentrated then water (10 mL) was added to the residue. The resulting mixture was extracted with EtOAc (3×10 mL) and the combined organic phase was washed with brine (5 mL), water (5 mL), dried over anhydrous Na₂SO₄, filtered and concentrated. The residue was purified by flash silica gel chromatography (eluent of 0~3% EtOAc in PE). 1-(tert-butyl) 4-methyl 5-fluoro-2-methylterephthalate (460 mg, 58% yield) as colorless oil. ¹H NMR (400 MHz, DMSO-d₆) δ=7.76 (d, J=7.2 Hz, 1H), 7.56 (d, J=11.2 Hz, 1H), 3.86 (s, 3H), 2.45 (s, 3H), 1.54 (s, 9H).

Step 3. To a solution of 1-(tert-butyl) 4-methyl 5-fluoro-2-methylterephthalate (400 mg, 1.49 mmol) in DCM (7 mL) was added TFA (1.02 g, 8.95 mmol). The mixture was stirred at 25° C. for 2 hrs. The reaction mixture was concentrated directly to give 5-fluoro-4-methoxycarbonyl-2-methyl-benzoic acid (400 mg, 54% yield) as a white solid, which was used in the next step without further purification. ¹H NMR (400 MHz, DMSO-d₆) δ=7.75 (d, J=7.2 Hz, 1H), 7.60 (d, J=11.2 Hz, 1H), 3.83 (s, 3H), 2.46 (s, 3H).

### Synthesis of 3,5-difluoro-4-methoxycarbonyl-benzoic acid

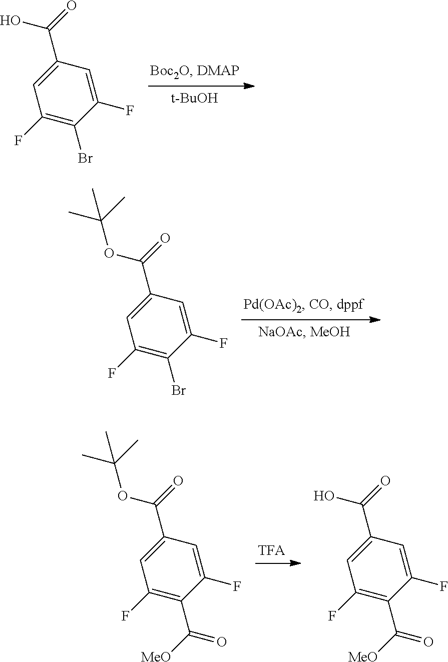

Step 1. To a solution of 4-bromo-3,5-difluoro-benzoic acid (1.00 g, 4.22 mmol) in DCM (10 mL) was added t-BuOH (5 mL, 52.3 mmol), Boc₂O (1.84 g, 8.44 mmol) and DMAP (155 mg, 1.27 mmol) at 25° C. The mixture was stirred at 25° C. for 16 hrs. The mixture was diluted with DCM (40 mL) and washed with water (2×20 mL). The organic layer was dried over Na₂SO₄ and concentrated. The crude product was purified by column chromatography on silica gel (100% PE) to give tert-butyl 4-bromo-3,5-difluoro-benzoate (1.10 g, 89% yield) as a white solid. ¹H NMR (DMSO-d₆, 400 MHz) δ_{H}=7.65 (d, J=7.2 Hz, 2H), 1.54 (s, 9H).

Step 2. To a stirring solution of tert-butyl 4-bromo-3,5-difluoro-benzoate (900 mg, 3.07 mmol) in methanol (9 mL) was added DPPF (170 mg, 0.31 mmol), Pd(OAc)₂ (69 mg, 0.31 mmol) and sodium acetate (756 mg, 9.21 mmol) at 25° C. The reaction mixture was degassed and purged with N₂ for 3 times then heated to 100° C. under CO gas atmosphere (1 MPa) and stirred for 12 hrs. The reaction mixture was concentrated and water (30 mL) was added to the residue. The resulting mixture was extracted with EtOAc (3×50 mL). The combined organic phase was washed with brine (30 mL), dried over anhydrous Na₂SO₄, filtered and concentrated. The crude product was purified by column chromatography on silica gel (EtOAc in PE, 1%) to give O₄-tert-butyl 01-methyl 2,6-difluorobenzene-1,4-dicarboxylate (816 mg, 98% yield) as colorless oil. ¹H NMR (DMSO-d₆, 400 MHz) δ_{H}=7.66 (d, J=8.4 Hz, 2H), 3.92 (s, 3H), 1.55 (s, 9H).

Step 3. A solution of (4-tert-butyl 01-methyl 2,6-difluorobenzene-1,4-dicarboxylate (816 mg, 3.00 mmol) in DCM (8 mL) and TFA (8 mL, 3.00 mmol) was stirred at 25° C. for 10 hrs. The reaction mixture was concentrated directly to give 3,5-difluoro-4-methoxycarbonyl-benzoic acid (598 mg, 92% yield) as a white solid, which was used in the next step directly. ¹H NMR (DMSO-d₆, 400 MHz) δ_{H}=7.68 (d, J=8.4 Hz, 2H), 3.92 (s, 3H).

### Synthesis of 4-(methoxycarbonyl)-2,5-dimethylbenzoic acid

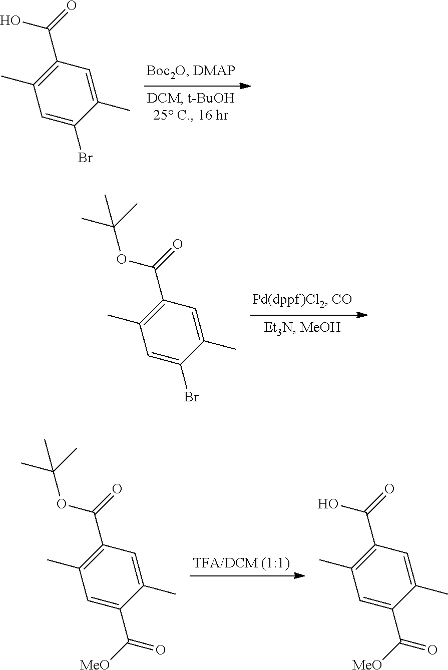

Step 1. To a solution of 4-bromo-2,5-dimethylbenzoic acid (1.00 g, 4.37 mmol) in DCM (20 mL) was added t-BuOH (10.0 mL, 105 mmol), Boc₂O) (1.91 g, 8.73 mmol) and DMAP (0.160 g, 1.31 mmol) at 25° C. The mixture was stirred at 25° C. for 16 hrs. Aq. NaHCO₃ (40 mL) was added to the residue. The resulting mixture was extracted with DCM (3×10 mL). The combined organic phase was washed with brine (10 mL), water (10 mL), dried over anhydrous Na₂SO₄, filtered and concentrated. The residue was purified by flash silica gel chromatography (eluent of 0~1% EtOAc in PE) to give tert-butyl 4-bromo-2,5-dimethylbenzoate (470 mg, 38% yield) as colorless oil. ¹H NMR (400 MHz, CHLOROFORM-d) δ=7.66 (s, 1H), 7.40 (s, 1H), 2.49 (s, 3H), 2.37 (s, 3H), 1.58 (s, 9H).

Step 2. To a solution of tert-butyl 4-bromo-2,5-dimethylbenzoate (470 mg, 1.65 mmol) in Methanol (10 mL) was added TEA (0.71 mL, 4.94 mmol) and Pd(dppf)Cl₂ (60 mg, 0.0824 mmol) at 25° C. The suspension was degassed and purged with CO (50 psi) for 3 times. The mixture was stirred at 50° C. for 16 hrs. The reaction mixture was concentrated. Water (10 mL) was added to the residue. The resulting mixture was extracted with EtOAc (3×10 mL). The combined organic phase was washed with brine (5 mL), water (5 mL), dried over anhydrous Na₂SO₄, filtered and concentrated. The residue was purified by flash silica gel chromatography (eluent of 0~3% EtOAc in PE) to give 1-(tert-butyl) 4-methyl 2,5-dimethylterephthalate (320 mg, 74% yield) as colorless oil. ¹H NMR (400 MHz, CHLOROFORM-d) δ=7.76 (s, 1H), 7.66 (s, 1H), 3.92 (s, 3H), 2.58 (s, 3H), 2.55 (s, 3H), 1.62 (s, 9H).

Step 3. To a solution of 1-(tert-butyl) 4-methyl 2,5-dimethylterephthalate (320 mg, 1.21 mmol) in DCM (2 mL) was added TFA (2 mL) at 25° C. The mixture was stirred at 25° C. for 16 hrs. The reaction mixture was concentrated directly to give 4-(methoxycarbonyl)-2,5-dimethylbenzoic acid (250 mg, 99% yield) as colorless oil, which was used in the next step directly. ¹H NMR (400 MHz, CHLOROFORM-d) δ=7.91 (s, 1H), 7.78 (s, 1H), 3.92 (s, 3H), 2.61 (d, J=17.6 Hz, 6H).

### Example 109. Synthesis of 4-((5-(2-(2-aminopyridin-3-yl)-5-phenyl-3H-imidazo[4,5-b]pyridin-3-yl)pyridin-2-yl)carbamoyl)-2-methylbenzoic acid (Compound 118)

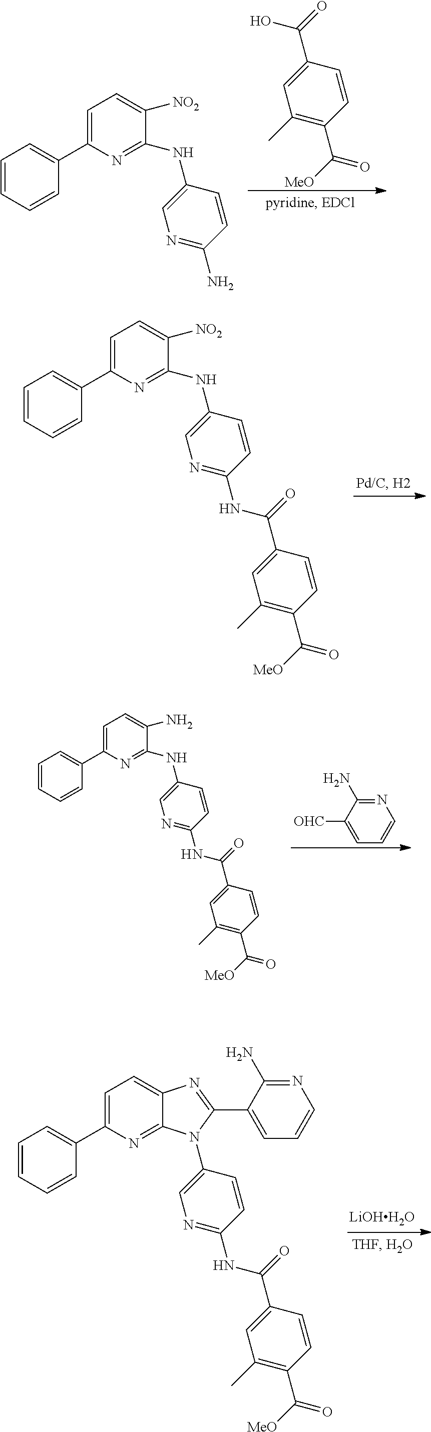

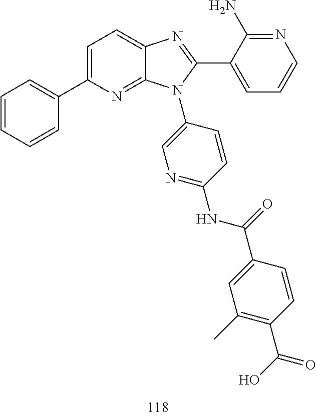 [SMILES: Cc1cc(C(=O)Nc2ccc(-n3c(-c4cccnc4N)nc4ccc(-c5ccccc5)nc43)cn2)ccc1C(=O)O]

Step 1. To a solution of N₅-(3-nitro-6-phenylpyridin-2-yl)pyridine-2,5-diamine (300 mg, 2.09 mmol) and 4-methoxycarbonyl-3-methyl-benzoic acid (227 mg, 1.17 mmol) in Pyridine (2 mL) was added EDCI (561 mg, 2.93 mmol). The mixture was heated to 80° C. and stirred for 1 hr at which point it was concentrated directly. Water (20 mL) was added and the product was extracted with EtOAc (3×20 mL). The combined organic phase was washed with brine (15 mL), dried over anhydrous Na₂SO₄, filtered and concentrated in vacuo. The residue was purified by flash silica gel chromatography (eluent of 0%~4% MeOH/DCM) to give methyl 2-methyl-4-((5-((3-nitro-6-phenylpyridin-2-yl)amino)pyridin-2-yl)carbamoyl)benzoate (160 mg, 28% yield) as a yellow solid. JH NMR (400 MHz, CHLOROFORM-d) δ=10.26 (s, 1H), 8.72 (d, J=2.4 Hz, 2H), 8.62 (d, J=8.8 Hz, 1H), 8.51 (d, J=8.8 Hz, 1H), 8.26 (dd, J=2.4, 9.2 Hz, 1H), 8.06-8.01 (m, 3H), 7.86-7.79 (m, 2H), 7.55-7.50 (m, 3H), 7.38 (d, J=8.8 Hz, 1H), 3.94 (s, 3H), 2.69 (s, 3H).

Step 2. To a solution of methyl 2-methyl-4-((5-((3-nitro-6-phenylpyridin-2-yl)amino)pyridin-2-yl)carbamoyl)benzoate (160 mg, 0.331 mmol) in methanol (3 mL) and THF (3 mL) was added wet Pd/C (0.20 g, 10% purity) under Ar. The reaction mixture was degassed and purged with H₂ for 3 times then stirred at 25° C. for 12 hrs under H₂ (15 Psi). The mixture was filtered through celite and washed (MeOH in DCM=20%, 4×10 mL). The filtrate was concentrated in vacuo to give methyl 4-((5-((3-amino-6-phenylpyridin-2-yl)amino)pyridin-2-yl)carbamoyl)-2-methylbenzoate (100 mg, 60% yield) as a yellow solid. ¹H NMR (400 MHz, DMSO-d₆) δ=10.85 (s, 1H), 9.06 (s, 1H), 8.22-8.19 (m, 2H), 8.17 (S, 1H), 8.07 (s, 1H), 8.00-7.96 (m, 4H), 7.46 (t, J=7.6 Hz, 2H), 7.38-7.32 (m, 2H), 7.08 (d, J=8.0 Hz, 1H), 5.36 (s, 2H), 3.93 (s, 3H), 2.65 (s, 3H).

Step 3. To a solution of methyl 4-((5-((3-amino-6-phenylpyridin-2-yl)amino)pyridin-2-yl)carbamoyl)-2-methylbenzoate (100 mg, 0.221 mmol) in acetic acid (2 mL) was added 2-aminopyridine-3-carbaldehyde (40 mg, 0.331 mmol) under N₂. The reaction mixture was degassed and purged with O₂ for 3 times then heated to 80° C. and stirred at temperature for 2 hrs. The reaction mixture was concentrated in vacuo and the residue was purified by flash silica gel chromatography (eluent of 0%~4% MeOH/DCM) to give methyl 4-((5-(2-(2-aminopyridin-3-yl)-5-phenyl-3H-imidazo[4,5-b]pyridin-3-yl)pyridin-2-yl)carbamoyl)-2-methylbenzoate (65 mg, 44% yield) as a brown solid. ¹H NMR (400 MHz, DMSO-d₆) δ=9.89 (s, 1H), 8.39 (dd, J=8.8, 19.2 Hz, 1H), 8.28 (dd, J=2.0, 4.8 Hz, 1H), 8.13-8.09 (m, 3H), 8.09-8.04 (m, 3H), 7.97 (s, 1H), 7.66-7.57 (m, 3H), 7.56-7.42 (m, 3H), 6.80 (dd, J=4.8, 7.6 Hz, 1H), 3.92 (s, 3H), 2.63 (s, 3H).

Step 4. To a solution of methyl 4-((5-(2-(2-aminopyridin-3-yl)-5-phenyl-3H-imidazo[4,5-b]pyridin-3-yl)pyridin-2-yl)carbamoyl)-2-methylbenzoate (65.0 mg, 0.118 mmol) in THF (3 mL) was added water (2 mL), LiOH·H₂O (1.00 g, 23.5 mmol) and NaOH (72.2 mg, 1.81 mmol). The mixture was stirred at 35° C. for 12 hrs then concentrated directly and purified by prep-HPLC (Column: Welch Xtimate C18 40*200 mm 7 μm; mobile phase: [water (FA)-ACN]; B %: 10%-50%: 25 min) to give 4-((5-(2-(2-aminopyridin-3-yl)-5-phenyl-3H-imidazo[4,5-b]pyridin-3-yl)pyridin-2-yl)carbamoyl)-2-methylbenzoic acid 118 (19.0 mg, 18% yield) as a yellow solid. ¹H NMR (400 MHz, DMSO-d₆) δ=11.13 (s, 1H), 8.54 (d, J=2.4 Hz, 1H), 8.37 (d, J=8.8 Hz, 1H), 8.31 (d, J=8.4 Hz, 1H), 8.10-8.02 (m, 5H), 7.98 (s, 1H), 7.90 (s, 2H), 7.52-7.46 (m, 2H), 7.44-7.39 (m, 2H), 6.84 (s, 2H), 6.54 (dd, J=4.8, 7.6 Hz, 1H), 2.59 (s, 3H). HPLC Rₜ=2.782 min in 8.0 min chromatography, purity 96%. LCMS Rₜ=1.891 min in 2 min chromatography, purity 96%, MS ESI calcd, for 539.26 [M+H]⁺ 540.26, found 542.2.

Compounds Prepared in Accordance with General Scheme 2

The compounds herein (such as a compound of Formula I, or other compounds in Section I) may be prepared according to General Scheme 2, for example, from an intermediate as prepared in “Synthesis of additional intermediates for Table 2 compounds,” an intermediate that is commercially available, an intermediate as prepared elsewhere in this application or an intermediate that can be synthesized via a method known to those skilled in the art. An exemplary full synthesis is provided in Example 109 (Compound 118). Compounds prepared in accordance with General Scheme 2 are provided in Table 2 below.

TABLE 2

| | Compound | | | |
|---|---|---|---|---|
| Example | No. | Structure | 1H NMR | LCMS |
| 110 | 145 | 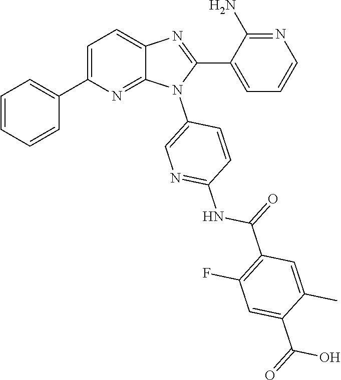 [SMILES: Cc1cc(C(=O)Nc2ccc(-n3c(-c4cccnc4N)nc4ccc(-c5ccccc5)nc43)cn2)c(F)cc1C(=O)O] | ¹H NMR (400 MHz, DMSO-d₆) δ = 11.17 (s, 1H), 8.51 (d, J = 2.4 Hz, 1H), 8.35 (d. J = 8.8 Hz, 1H), 8.30 (d, J = 8.4 Hz, 1H), 8.10- 8.00 (m, 5H), 7.65- 7.57 (m, 2H), 7.52-7.45 (m, 2H), 7.44-7.38 (m, 2H), 6.83 (s, 2H), 6.54 (dd, J = 4.8. 7.6 Hz, 1H), 2.52 (s, 3H). | MS ESI caled. for 559.18, [M + H]⁺ 560.18, found 560.2. |
| 111 | 146 | 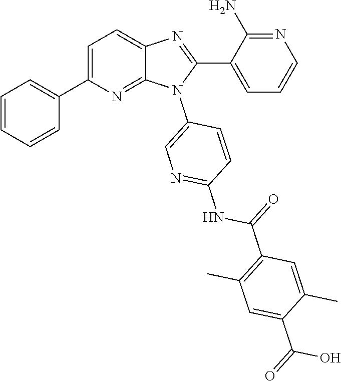 [SMILES: Cc1cc(C(=O)Nc2ccc(-n3c(-c4cccnc4N)nc4ccc(-c5ccccc5)nc43)cn2)c(C)cc1C(=O)O] | ¹H NMR (400 MHz, DMSO-d₆) δ_{H} = 11.11 (s, 1H), 8.49 (d, J = 2.4 Hz, 1H), 8.36 (d, J = 8.8 Hz, 1H), 8.30 (d, J = 8.4 Hz, 1H), 8.09- 8.00 (m, 5H), 7.71 (s, 1H), 7.52-7.44 (m, 3H), 7.42 (dd, J = 2.0, 7.6 Hz, 2H), 6.79 (s, 2H), 6.55 (dd, J = 4.8, 7.6 Hz, 1H), 2.52 (s, 3H), 2.40 (s, 3H) | MS ESI caled. for 555.20 [M + H]⁺ 556.20, found 556.2 |
| 112 | 147 | 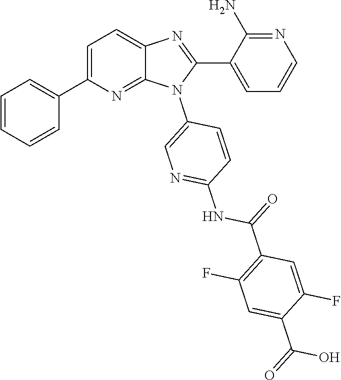 [SMILES: Nc1ncccc1-c1nc2ccc(-c3ccccc3)nc2n1-c1ccc(NC(=O)c2cc(F)c(C(=O)O)cc2F)nc1] | ¹H NMR (400 MHz, DMSO-d₆) δ_{H} = 11.42 (s, 1H), 8.32 (d, J = 8.4 Hz, 1H), 8.20 (d, J = 8.4 Hz, 1H), 8.06-7.97 (m, 5H), 7.73 (dt, J = 5.6, 10.0 Hz, 2H), 7.51-7.44 (m. 2H), 7.41 (d, J = 7.2 Hz, 1H), 7.28- 7.15 (m, 3H), 6.50 (dd, J = 4.8, 7.6 Hz, 1H), 2.12 (s, 3H) | MS ESI calcd. for 577.17, [M + H]⁺ 578.17, found 578.3. |
| 113 | 148 | 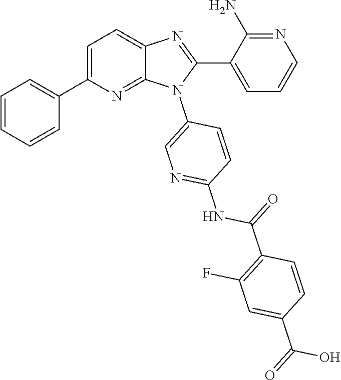 [SMILES: Nc1ncccc1-c1nc2ccc(-c3ccccc3)nc2n1-c1ccc(NC(=O)c2ccc(C(=O)O)cc2F)nc1] | ¹H NMR (DMSO- d₆, 400 MHz) δ_{H} = 11.33 (s. 1H), 8.32 (d, J = 8.4 Hz, 1H), 8.22 (d, J = 9.2 Hz, 1H), 8.06- 7.96 (m, 5H), 7.88- 7.80 (m, 2H), 7.77 (d, J = 10.4 Hz, 1H), 7.51- 7.44 (m, 2H), 7.41 (d. J = 7.2 Hz, 1H), 7.23 (dd, J = 1.6, 7.6 Hz, 1H), 7.17 (s, 2H), 6.49 (dd, J = 4.8. 7.6 Hz, 1H). 2.12 (s, 3H). | MS ESI calcd. for 559.18, [M + H]⁺ 560.18. found 560.2 |
| 114 | 149 | 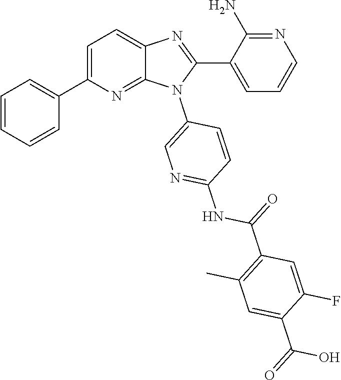 | ¹H NMR (400 MHz, DMSO-d₆) δ = 11.28-11.16 (m, 1H), 8.32 (d, J = 8.4 Hz, 1H), 8.22 (d, J = 8.4 Hz, 1H), 8.06- 7.93 (m, 5H), 7.70 (d, J = 6.8 Hz, 1H). 7.50-7.36 (m, 4H), 7.24 (dd, J = 1.6, 7.6 Hz. 1H), 7.14 (s, 2H), 6.49 (dd, J = 4.8, 7.6 Hz, 1H), 2.39 (s, 3H), 2.12 (s, 3H). | MS ESI calcd. for 573.19 [M + H]⁺ 574.19, found 574.1 |
| 115 | 117 | 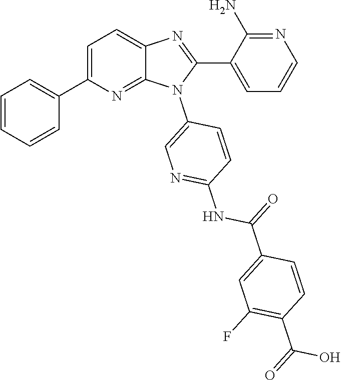 [SMILES: Nc1ncccc1-c1nc2ccc(-c3ccccc3)nc2n1-c1ccc(NC(=O)c2ccc(C(=O)O)c(F)c2)nc1] | ^{I}H NMR (400 MHz, DMSO-d₆) δ = 11.28-11.16 (m, 1H), 8.32 (d, J = 8.4 Hz, 1H), 8.22 (d, J = 8.4 Hz, 1H), 8.06- 7.93 (m, 5H), 7.70 (d, J = 6.8 Hz, 1H), 7.50-7.36 (m, 4H), 7.24 (dd, J = 1.6, 7.6 Hz, 1H), 7.14 (s, 2H), 6.49 (dd, J = 4.8, 7.6 Hz, 1H), 2.39 (s, 3H), 2.12 (s, 3H). | MS ESI calcd. for 545.16 [M + H]⁺ 546.16, found 546.2. |
| 116 | 150 | 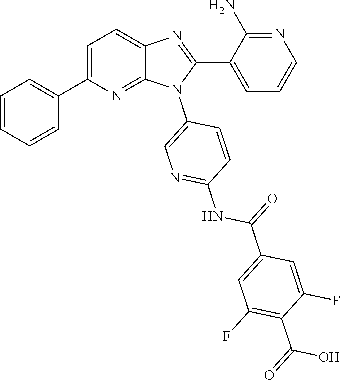 [SMILES: Nc1ncccc1-c1nc2ccc(-c3ccccc3)nc2n1-c1ccc(NC(=O)c2cc(F)c(C(=O)O)c(F)c2)nc1] | ¹H NMR (DMSO- d₆, 400 MHz) δ_{H} = 11.34 (s, 1H), 8.59 (d, J = 2.4 Hz, 1H), 8.34 (dd, J = 7.6, 8.8 Hz, 2H), 8.13-8.03 (m, 5H), 7.90-7.82 (m, 2H), 7.64 (d, J = 7.2 Hz, 1H), 7.52-7.46 (m, 2H), 7.45-7.39 (m, 1H), 6.69 (dd, J = 5.6, 7.2 Hz, 1H). | MS ESI calcd. for 563.15 [M + H]⁺ 564.15, found 564.1 |
| 117 | 151 | 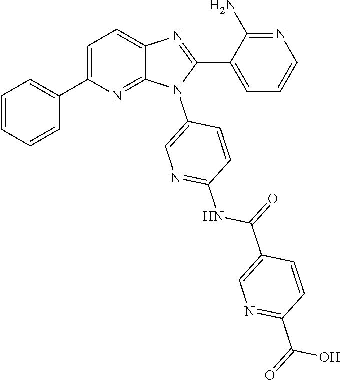 [SMILES: Nc1ncccc1-c1nc2ccc(-c3ccccc3)nc2n1-c1ccc(NC(=O)c2ccc(C(=O)O)nc2)nc1] | ¹H NMR (DMSO- d₆, 400 MHz) δ_{H} = 11.47 (s, 1H), 9.36- 9.07 (m, 1H), 8.59-8.45 (m, 2H), 8.42-8.26 (m, 2H), 8.19- 8.00 (m, 6H), 7.51- 7.38 (m, 4H), 6.82 (s, 2H), 6.54 (dd, J = 4.8, 7.6 Hz. 1H). | MS ESI calcd. for 528.17, [M + H]⁺ 529.17, found 529.1. |
| 118 | 152 | 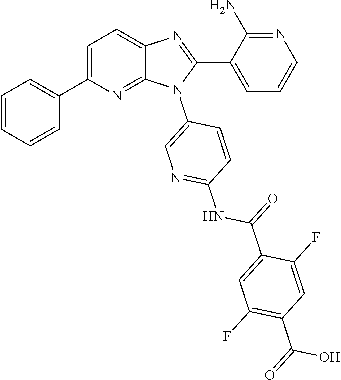 [SMILES: Nc1ncccc1-c1nc2ccc(-c3ccccc3)nc2n1-c1ccc(NC(=O)c2cc(F)c(C(=O)O)cc2F)nc1] | ¹H NMR (DMSO- d₆, 400 MHz) δ_{H} = 11.41 (s, 1H), 8.61 (s, 1H), 8.39-8.33 (m, 2H), 8.17- 8.05 (m, 6H), 7.96 (d, J = 6.8 Hz, 1H). 7.79-7.69 (m, 2H), 7.53- 7.47 (m, 2H), 7.46- 7.41 (m, 1H), 6.93 (t. J = 6.4 Hz, 1H). | MS ESI calcd. for 563.15 [M + H]⁺ 564.15, found 564.2 |
| 119 | 153 | 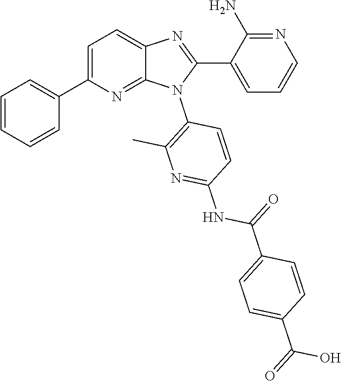 [SMILES: Cc1nc(NC(=O)c2ccc(C(=O)O)cc2)ccc1-n1c(-c2cccnc2N)nc2ccc(-c3ccccc3)nc21] | ¹H NMR (400 MHz, DMSO-d₆) δ = 13.24 (s, 1H), 11.25 (s, 1H), 8.32 (d, J = 8.4 Hz, 1H), 8.24 (d. J = 8.4 Hz, 1H), 8.17- 8.10 (m. 2H), 8.08- 7.94 (m, 7H), 7.51-7.34 (m, 3H), 7.23 (dd, J = 2.0, 8.0 Hz, 1H), 7.18 (s, 2H), 6.48 (dd, J = 4.8, 8.0 Hz, 1H), 2.14 (s, 3H | MS ESI calcd. for 541.19 [M + H]⁺ 542.19, found 542.1 |
| 120 | 154 | 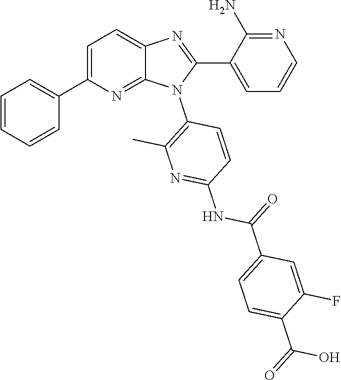 [SMILES: Cc1nc(NC(=O)c2ccc(C(=O)O)c(F)c2)ccc1-n1c(-c2cccnc2N)nc2ccc(-c3ccccc3)nc21] | ¹H NMR (400 MHz, DMSO-d₆) δ = 8.32 (d, J = 8.4 Hz, 1H), 8.23 (d. J = 8.4 Hz, 1H), 8.06-8.00 (m, 4H), 7.97 (d, J = 8.4 Hz, IH), 7.87- 7.78 (m, 2H), 7.73 (s, 1H), 7.51-7.45 (m, 2H), 7.41 (d, J = 7.2 Hz, 1H), 7.23 (dd, J = 1.6, 7.6 Hz, 1H), 7.18 (s, 2H), 6.49 (dd, J = 4.8, 7.6 Hz, 1H), 2.15 (s, 3H). | MS ESI calcd. for 559.18 [M + H]⁺ 560.18, found 560.2. |
| 121 | 155 | 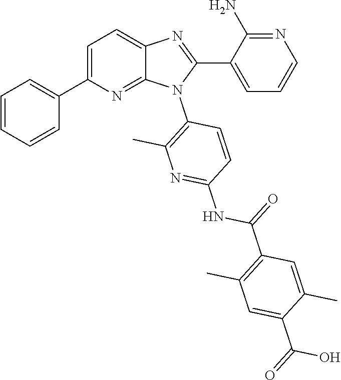 [SMILES: Cc1cc(C(=O)Nc2ccc(-n3c(-c4cccnc4N)nc4ccc(-c5ccccc5)nc43)c(C)n2)c(C)cc1C(=O)O] | ¹H NMR (400 MHz, DMSO-d₆) δ_{H} = 11.13 (s, 1H), 8.32 (d, J = 8.4 Hz, 1H), 8.23 (d, J = 8.4 Hz, 1H), 8.07-7.98 (m, 4H), 7.95 (d, J = 8.4 Hz, 1H), 7.71 (s, 1H), 7.52-7.44 (m, 3H), 7.43- 7.36 (m. 1H), 7.24 (dd, J = 1.6, 7.6 Hz, 1H), 7.15 (s, 2H), 6.49 (dd, J = 4.8, 7.6 Hz, 1H), 2.52 (s, 3H), 2.40 (s, 3H), 2.11 (s, 3H) | MS ESI calcd. for 569.22, [M + H]⁺ 570.22, found 570.2 |
| 122 | 156 | 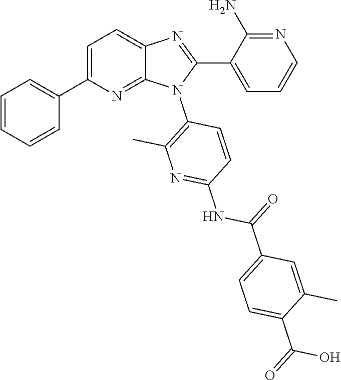 [SMILES: Cc1cc(C(=O)Nc2ccc(-n3c(-c4cccnc4N)nc4ccc(-c5ccccc5)nc43)c(C)n2)ccc1C(=O)O] | . ¹H NMR (DMSO-d₆, 400 MHz) δ_{H} = 11.15 (s, 1H), 8.33 (d, J = 8.4 Hz, 1H), 8.25 (d, J = 8.8 Hz, 1H), 8.06- 7.96 (m, 6H), 7.91 (d, J = 2.4 Hz, 2H), 7.52-7.44 (m, 2H), 7.41 (d. J = 7.2 Hz, 1H), 7.27-7.16 (m, 3H), 6.49 (dd, J = 4.8, 7.6 Hz, 1H), 2.60 (s, 3H), 2.15 (s, 3H). | MS ESI calcd. for 555.20, [M + H]⁺ 556.20, found 556.2. |

### Synthesis of Additional Intermediates for Table 3 Compounds

### Synthesis of Methyl 4-(1-bromoethyl)-3-fluoro-benzoate

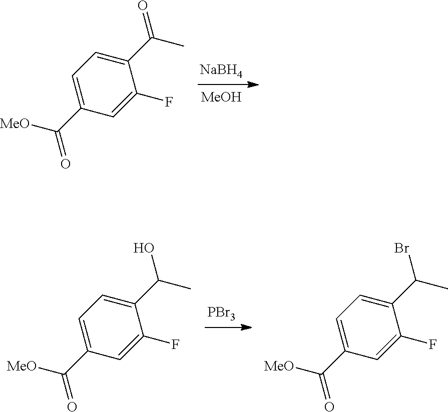

Step 1. To a solution of methyl 4-acetyl-3-fluoro-benzoate (200 mg, 1.02 mmol) in methanol (3.00 mL) was added NaBH₄ (77.0 mg, 2.04 mmol) at 0° C. under N₂. The mixture was warmed to 25° C. and stirred for 1 hr. 1 M HCl was added. H₂O (20 mL) was added and the product was extracted with EtOAc (3×20 mL). The organic phase was washed with brine (20 mL), dried over Na₂SO₄, filtered and concentrated in vacuo. The crude was purified by flash chromatography eluting with PE/EtOAc (from 0% to 20% of EtOAc in PE) to give methyl 3-fluoro-4-(1-hydroxyethyl)benzoate (160 mg, 79% yield) as colorless oil. ¹H NMR (400 MHz, DMSO-d₆) δ=7.80 (dd, J=1.6, 8.0 Hz, 1H), 7.71-7.65 (m, 1H), 7.61 (dd, J=1.2, 11.2 Hz, 1H), 5.53-5.47 (m, 1H), 5.05-4.97 (m, 1H), 3.85 (s, 3H), 1.34 (d, J=6.0 Hz, 3H).

Step 2. To a solution of methyl 3-fluoro-4-(1-hydroxyethyl)benzoate (160 mg, 0.807 mmol) in DCM (3.0 mL) was added PBr₃ (0.15 mL, 1.61 mmol) at 0° C. under N₂. The mixture was stirred at 25° C. for 2 hrs. NaHCO₃ (20 mL) was added. The mixture was extracted with DCM (20 mL×3). The organic phase was washed with brine (20 mL), dried over Na₂SO₄, filtered and concentrated in vacuo to give methyl 4-(1-bromoethyl)-3-fluoro-benzoate (60.0 mg, 28% yield) as yellow oil, which was directly used in the next step without purification. ¹H NMR (400 MHz, DMSO-d₆) δ=7.84-7.77 (m, 2H), 7.70 (d, J=10.8 Hz, 1H), 5.62 (q, J=6.8 Hz, 1H), 3.87 (s, 3H), 2.02 (d, J=6.8 Hz, 3H).

### Synthesis of Methyl 4-(1-bromo-2-methyl-propyl)benzoate

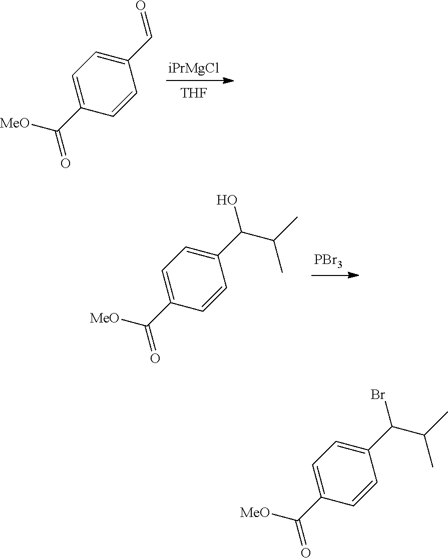

Step 1. A solution of methyl 4-formylbenzoate (5.00 g, 30.5 mmol) in tetrahydrofuran (25 mL) was cooled to −78° C. To this solution was added 2M/Isopropylmagnesium chloride (15 mL, 31.0 mmol) dropwise over 20 minutes. The reaction mixture was degassed and purged with N₂, 3 times, then stirred at −78° C. for 2 hrs. The reaction was then quenched by addition of saturated aqueous ammonium chloride (100 mL) and the product was extracted with ethyl acetate (3×150 mL). The organic layer was dried over magnesium sulfate, filtered and concentrated in vacuo. The residue was purified by flash silica gel chromatography (eluent of 0~9% EtOAc in PE) to give methyl 4-(1-hydroxy-2-methyl-propyl)benzoate (2.10 g, 33% yield) as yellow oil. ¹H NMR (400 MHz, DMSO-d₆) δ_{H}=7.91 (d, J=8.0 Hz, 2H), 7.43 (d, J=8.0 Hz, 2H), 5.28 (d, J=4.4 Hz, 1H), 4.38-4.32 (m, 1H), 3.84 (s, 3H), 1.86-1.77 (m, 1H), 0.85-0.73 (m, 6H).

Step 2. To a solution of methyl 4-(1-hydroxy-2-methyl-propyl)benzoate (2.10 g, 10.1 mmol) in DCM (21.0 mL) was added tribromophosphane (1.90 mL, 20.2 mmol) at 0° C. The reaction mixture was degassed and purged with N₂ for 3 times and the resulting mixture was stirred at 25° C. for 2 hrs. The reaction was quenched with saturated aq. NaHCO₃ (60 mL) drop-wise at 0° C. and then extracted with DCM (3×80 mL). The combined organic phase was washed with brine (40 mL), dried over anhydrous Na₂SO₄, filtered and concentrated in vacuo to give methyl 4-(1-bromo-2-methyl-propyl)benzoate (2.40 g, 88% yield) as yellow oil. ¹H NMR (400 MHz, DMSO-d₆) δ=7.94 (d, J=8.4 Hz, 2H), 7.58 (d, J=8.0 Hz, 2H), 5.11 (d, J=8.8 Hz, 1H), 3.85 (s, 3H), 2.36-2.27 (m, 1H), 1.12 (d, J=6.4 Hz, 3H), 0.78 (d, J=6.4 Hz, 3H).

### Synthesis of Methyl 4-(1-Bromoethyl)-2-Fluoro-Benzoate

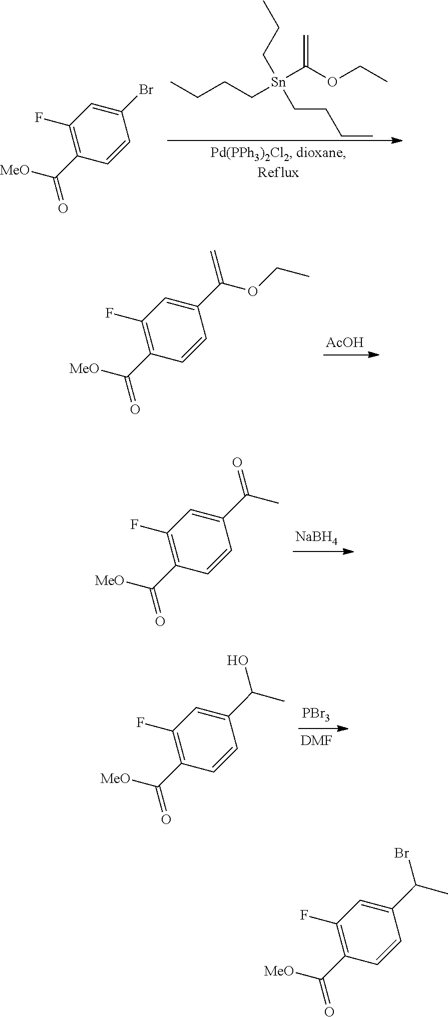

Step 1. To a mixture of methyl 4-bromo-2-fluoro-benzoate (3.70 g, 15.9 mmol) in 1,4-Dioxane (37 mL) was added tributyl(1-ethoxyvinyl) stannane (9.75 g, 27.0 mmol) followed by KF (2.76 g, 47.6 mmol). The mixture was purged with N₂ for 15 min and then Pd(PPh₃)₂C12 (1.11 g, 1.59 mmol) was added into it at 20° C. The mixture was degassed and purged with N₂ for 3 times and then heated to 100° C. and stirred for 7 hrs under N₂ atmosphere. Saturated aqueous KF (100 mL) was then added and the mixture was filtered through a celite pad. The product was extracted with EtOAc (3×150 mL) and the combined organic layers were washed with brine (2×50 mL), dried over Na₂SO₄, filtered and concentrated in vacuo. The residue was purified by flash silica gel chromatography (eluent of 0~3% EtOAc in PE) to give crude methyl 4-(1-ethoxyvinyl)-2-fluoro-benzoate (3.70 g, crude) as yellow oil. ¹H NMR (400 MHz, DMSO-d₆) δ=7.87 (t, J=8.0 Hz, 1H), 7.62-7.48 (m, 2H), 5.02 (d, J=3.2 Hz, 1H), 4.48 (d, J=2.8 Hz, 1H), 3.90 (q, J=7.2 Hz, 2H), 3.85 (s, 3H), 1.35 (1, J=7.2 Hz, 3H).

Step 2. To a solution of methyl 4-(1-ethoxyvinyl)-2-fluoro-benzoate (3.20 g, 14.3 mmol) in THF (12 mL) and water (12 mL) was added AcOH (12.0 mL, 14.3 mmol). The mixture was stirred at 25° C. for 12 hrs. Saturated aqueous NaOH (40 mL) was then added to the residue and the resulting mixture was extracted with EtOAc (3×60 mL). The combined organic phase was washed with brine (30 mL), dried over anhydrous Na₂SO₄, filtered and concentrated in vacuo. The residue was purified by flash silica gel chromatography (eluent of 0-10% EtOAc in PE) to give methyl 4-acetyl-2-fluoro-benzoate (2.40 g, 86% yield) as a white solid. ¹H NMR (400 MHz, DMSO-d₆) δ=8.04-7.97 (m, 1H), 7.89-7.79 (m, 2H), 3.89 (s, 3H), 2.63 (s, 3H).

Step 3. To a solution of methyl 4-acetyl-2-fluoro-benzoate (2.40 g, 12.2 mmol) in Methanol (24.0 mL) was added NaBH₄ (0.56 g, 14.8 mmol) at 0° C. The reaction mixture was degassed and purged with N₂ for 3 times then stirred at 25° C. for 0.5 hr. Saturated aqueous NH₄Cl (50 mL) was added to the residue and the resulting mixture was extracted with EtOAc (3×80 mL). The combined organic phase was washed with brine (50 mL), dried over anhydrous Na₂SO₄, filtered and concentrated in vacuo to give methyl 2-fluoro-4-(1-hydroxyethyl)benzoate (2.30 g, 95% yield) as yellow oil. ¹H NMR (400 MHz, DMSO-d₆) ÅH=7.84 (t, J=8.0 Hz, 1H), 7.32-7.23 (m, 2H), 5.46 (d, J=4.4 Hz, 1H), 4.83-4.73 (m, 1H), 3.84 (s, 3H), 1.32 (d, J=6.4 Hz, 3H).

Step 4. To a solution of methyl 2-fluoro-4-(I-hydroxyethyl)benzoate (1.00 g, 5.05 mmol) in DMF (10 mL) was added tribromophosphane (0.95 mL, 10.1 mmol) at 0° C. The reaction mixture was degassed and purged with N₂ for 3 times. The resulting mixture was stirred at 25° C. for 2 hrs and then quenched with saturated aq. NaHCO₃ (40 mL) drop-wise at 0° C. The resulting mixture was extracted with EtOAc (3×50 mL) and the combined organic phase was washed with brine (20 mL), dried over anhydrous Na₂SO₄, filtered and concentrated in vacuo. The residue was purified by flash silica gel chromatography (eluent of 0-10% EtOAc in PE) to give methyl 4-(1-bromoethyl)-2-fluoro-benzoate (1.20 g, 91% yield) as yellow oil. ¹H NMR (400 MHz, DMSO-d₆) δ=7.88 (t, J=8.0 Hz, 1H), 7.57-7.38 (m, 2H), 5.51 (q, J=6.8 Hz, 1H), 3.85 (s, 3H), 1.98 (d, J=6.8 Hz, 3H).

### Synthesis of 3-[3-[4-(azetidin-3-yl)phenyl]-5-phenyl-imidazo[4,5-b]pyridin-2-yl]pyridin-2-amine

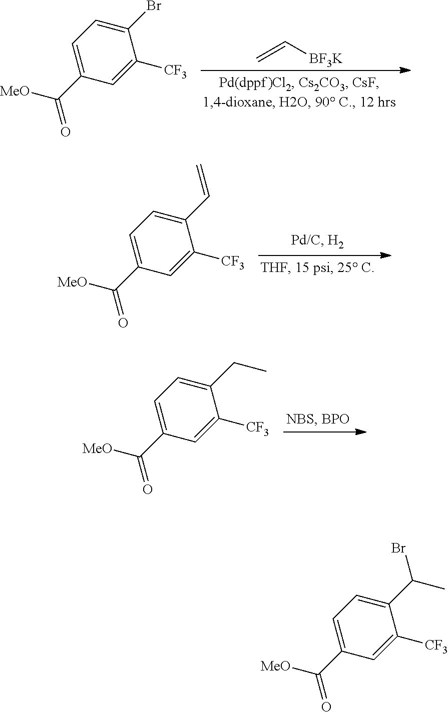

Step 1. To a solution of methyl 4-bromo-3-(trifluoromethyl)benzoate (2.00 g, 7.07 mmol) in 1,4-Dioxane (20 mL) and water (5 mL) was added potassium trifluoro (vinyl) boranuide (1.04 g, 7.77 mmol), Pd(dppf)Cl₂ (248 mg, 0.350 mmol), CsF (41 mg, 0.710 mmol) and Cs₂CO₃ (6.91 g, 21.2 mmol) at 25° C. The mixture was heated to 90° C. and stirred for 12 hrs. Water (10 mL) was then added and the resulting mixture was extracted with DCM (3×20 mL). The combined organic phase was washed with brine (10 mL), dried over anhydrous Na₂SO₄, filtered and concentrated in vacuo. The residue was purified by flash silica gel chromatography (eluent of 0~2% EtOAc in PE) to afford methyl 3-(trifluoromethyl)-4-vinyl-benzoate (1.34 g, 82% yield) as a yellow solid. ¹H NMR (DMSO-d₆, 400 MHz) δ_{H}=8.19-8.11 (m, 2H), 7.98 (d, J=8.4 Hz, 1H), 7.07-6.96 (m, 1H), 6.07 (d, J=17.2 Hz, 1H), 5.66 (d, J=11.6 Hz, 1H), 3.88 (s, 3H).

Step 2. To a solution of methyl 3-(trifluoromethyl)-4-vinyl-benzoate (1.34 g, 5.82 mmol) in THE (6 mL) was added Pd/C (500 mg, 10% purity) under Ar atmosphere. The suspension was degassed and purged with H₂ for 3 times. The reaction mixture was stirred under H₂ (20 Psi) at 25° C. for 12 hrs. The reaction mixture was filtered through a pad of Celite. The filter cake was washed with MeOH/DCM (1:1, 200 mL). The resulting suspension was concentrated directly to afford methyl 4-ethyl-3-(trifluoromethyl)benzoate (1.17 g, 87% yield) as a colorless oil. H NMR (DMSO-d₆, 400 MHz) δ_{H}=8.21-8.08 (m, 2H), 7.68 (d, J=8.0 Hz, 1H), 3.88 (s, 3H), 2.93-2.76 (m, 2H), 1.22 (t, J=7.6 Hz, 3H).

Step 3. To a solution of methyl 4-ethyl-3-(trifluoromethyl)benzoate (200 mg, 0.860 mmol) in DCE (4 mL) was added NBS (169 mg, 0.95 mmol) and BPO (31.0 mg, 0.130 mmol) at 25° C. The mixture was heated to 95° C. and stirred for 12 hrs under N₂ atmosphere. The reaction was then diluted with water (3 mL) and the resulting mixture was extracted with DCM (3×6 mL). The combined organic phase was washed with brine (3 mL), dried over anhydrous Na₂SO₄, filtered and concentrated in vacuo. The residue was purified by combi-flash column (EtOAc in PE, 0%~2%) to afford methyl 4-(1-bromoethyl)-3-(trifluoromethyl)benzoate (267 mg, 99% yield) as colorless oil. ¹H NMR (DMSO-d₆, 400 MHz) δ_{H}=8.30-8.24 (m, 1H), 8.22-8.11 (m, 2H), 5.59-5.48 (m, 1H), 3.90 (s, 3H), 2.04 (d, J=6.8 Hz, 3H).

### Example 123, Example 124, Example 125 and Example 126. Synthesis of methyl 4-[(1R)-1-[3-[4-[2-(2-amino-3-pyridyl)-5-phenyl-imidazo[4,5-b]pyridin-3-yl]phenyl]azetidin-1-yl]ethyl]benzoate (Compound 157)

### Synthesis of methyl 4-[(1S)-1-[3-[4-[2-(2-amino-3-pyridyl)-5-phenyl-imidazo[4,5-b]pyridin-3-yl]phenyl]azetidin-1-yl]ethyl]benzoate (Compound 158)

### Synthesis of 4-[(1R)-1-[3-[4-[2-(2-amino-3-pyridyl)-5-phenyl-imidazo]4,5-b]pyridin-3-yl]phenyl]azetidin-1-yl]ethyl]benzoic acid (Compound 125)

### Synthesis of 4-[(1S)-1-[3-[4-[2-(2-amino-3-pyridyl)-5-phenyl-imidazo[4,5-b]pyridin-3-yl]phenyl]azetidin-1-yl]ethyl]benzoic acid (Compound 126)

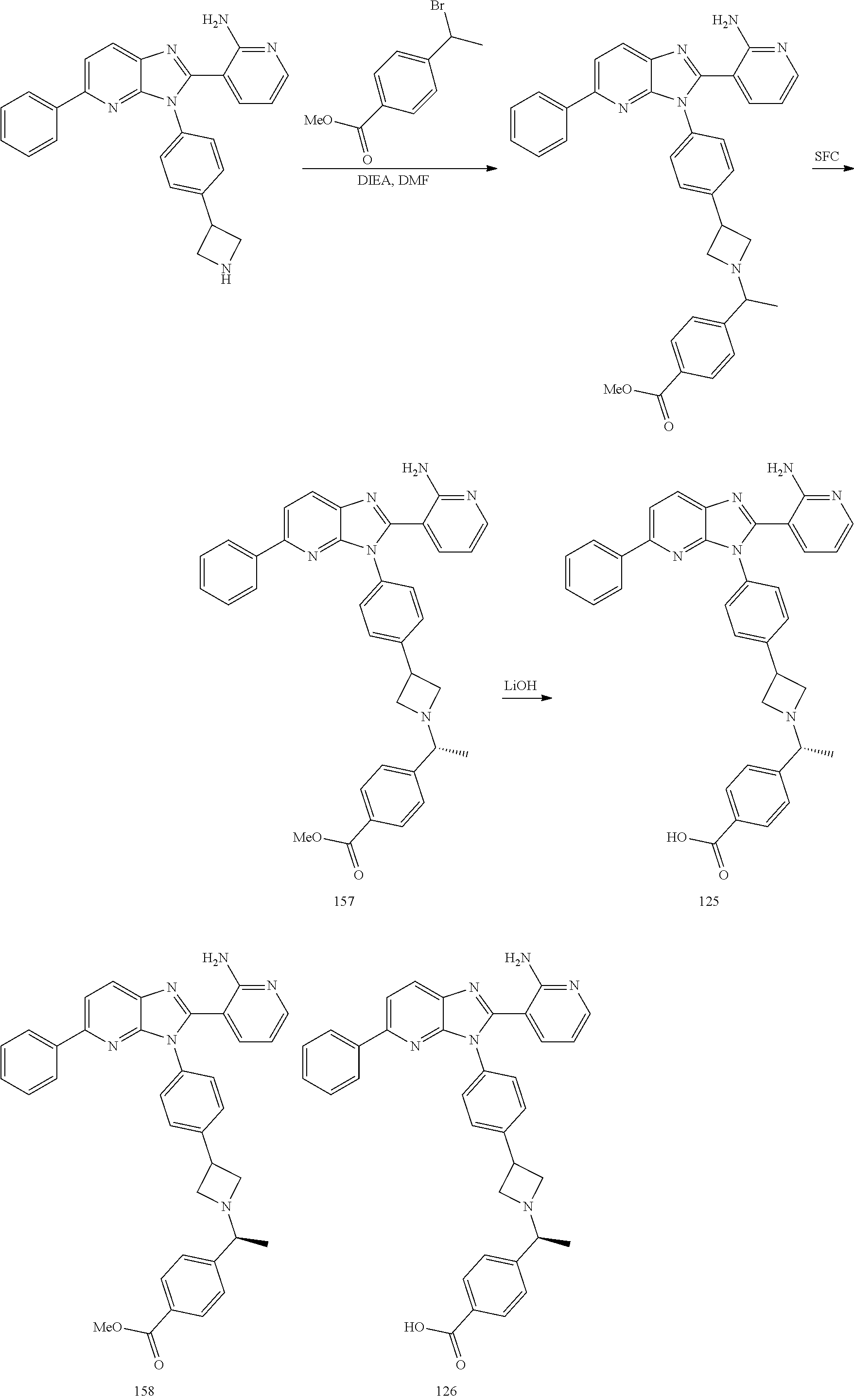

Step 1. To a solution of 3-[3-[4-(azetidin-3-yl)phenyl]-5-phenyl-imidazo[4,5-b]pyridin-2-yl]pyridin-2-amine (161 mg, 0.385 mmol) in DMF (3.00 mL) was added methyl 4-(1-bromoethyl)benzoate (94.0 mg, 0.385 mmol) and DIEA (0.210 mL, 1.15 mmol) at 25° C. under N₂. The mixture was stirred at 25° C. for 16 hrs at which point H₂O (10 mL) was added and the product was extracted with EtOAc (3×10 mL). The organic phase was washed with brine (10 mL×3), dried over Na₂SO₄, filtered and concentrated in vacuo. The crude was purified by flash chromatography eluting with EtOAc/MeOH (from 0% to 50% of MeOH in EtOAc) to give methyl 4-[1-[3-[4-[2-(2-amino-3-pyridyl)-5-phenyl-imidazo[4,5-b]pyridin-3-yl]phenyl]azetidin-1-yl]ethyl]benzoate (200 mg, 90% yield) as yellow oil. The crude was purified by SFC (DAICEL CHIRALCEL OJ (250 mm*30 mm, 10 μm) CO₂-EtOH (0.1% NH₃H₂O))

Isomer 1. Compound 157 methyl 4-[(1R)-1-[3-[4-[2-(2-amino-3-pyridyl)-5-phenyl-imidazo[4,5-b]pyridin-3-yl]phenyl]azetidin-1-yl]ethyl]benzoate 157 (54.0 mg, 27% yield) as a yellow solid. JH NMR (400 MHz, CHLOROFORM-d) δ=8.13 (d, J=8.0 Hz, 1H), 8.07 (dd, J=1.6, 7.6 Hz, 1H), 8.01 (dd, J=1.6, 7.6 Hz, 4H), 7.80 (d, J=7.6 Hz, 1H), 7.48-7.36 (m, 9H), 7.12 (dd, J=1.6, 7.6 Hz, 1H), 6.58 (s, 2H), 6.39 (dd, J=4.8, 7.6 Hz, 1H), 3.91 (s, 3H), 3.89-3.83 (m, 1H), 3.80-3.74 (m, 1H), 3.60 (t, J=7.2 Hz, 1H), 3.50-3.43 (m, 1H), 3.25 (t, J=7.2 Hz, 1H), 3.18-3.12 (m, 1H), 1.28 (d, J=6.8 Hz, 3H). HPLC Rₜ=4.193 min in 8 min chromatography, purity 100%. LCMS Rₜ=1.830 min in 4 min chromatography, purity 99.6%, MS ESI calcd, for 580.26, [M+H]⁺ 581.26, found 581.4.

Isomer 2. Compound 158 methyl 4-[(1S)-1-[3-[4-[2-(2-amino-3-pyridyl)-5-phenyl-imidazo[4,5-b]pyridin-3-yl]phenyl]azetidin-1-yl]ethyl]benzoate 158 (54 mg, 0.0930 mmol, 27% yield) as a yellow solid. ¹H NMR (400 MHz, CHLOROFORM-d) δ=8.13 (d, J=8.4 Hz, 1H), 8.07 (dd, J=1.6, 4.8 Hz, 1H), 8.04-7.98 (m, 4H), 7.80 (d, J=8.4 Hz, 1H), 7.48-7.36 (m, 9H), 7.11 (dd, J=1.6, 7.6 Hz, 1H), 6.58 (s, 2H), 6.39 (dd, J=4.4, 7.6 Hz, 1H), 3.91 (s, 3H), 3.87 (t, J=6.8 Hz, 1H), 3.77 (t, J=6.8 Hz, 1H), 3.64-3.57 (m, 1H), 3.51-3.42 (m, 1H), 3.25 (t, J=6.8 Hz, 1H), 3.15 (t, J=6.8 Hz, 1H), 1.28 (d, J=6.8 Hz, 3H). HPLC Rₜ=4.189 min in 8 min chromatography, purity 100%. LCMS Rₜ=1.859 min in 4 min chromatography, purity 98.6%, MS ESI calcd, for 580.26, [M+H]⁺ 581.26, found 581.4.

Step 2. To a solution of 4-[1-[3-[4-[2-(2-amino-3-pyridyl)-5-phenyl-imidazo[4,5-b]pyridin-3-yl]phenyl]azetidin-1-yl]ethyl]benzoic acid (54.0 mg, 0.095 mmol) in THF (1.00 mL) and water (1.00 mL) was added lithium hydroxide hydrate (10.0 mg, 0.238 mmol) at 25° C. The mixture was stirred at 25° C. for 16 hrs at which point the crude material was purified by prep-HPLC (Welch Xtimate C18 150*30 mm*5 μm water (FA)-ACN)

Isomer 1. Compound 125. 4-[(1R)-1-[3-[4-[2-(2-amino-3-pyridyl)-5-phenyl-imidazo[4,5-b]pyridin-3-yl]phenyl]azetidin-1-yl]ethyl]benzoic acid 125 (21.0 mg, 39% yield) as a yellow solid. ¹H NMR (400 MHz, DMSO-d₆) δ=8.26 (d, J=8.4 Hz, 1H), 8.04-7.97 (m, 4H), 7.90 (d, J=8.0 Hz, 2H), 7.55-7.51 (m, 2H), 7.50-7.43 (m, 6H), 7.42-7.37 (m, 1H), 7.20 (dd, J=2.0, 7.6 Hz, 1H), 6.97-6.90 (m, 2H), 6.42 (dd, J=4.8, 7.6 Hz, 1H), 3.76-3.65 (m, 2H), 3.58-3.46 (m, 2H), 3.23-3.06 (m, 2H), 1.18 (d, J=6.4 Hz, 3H). HPLC Rₜ=2.602 min in 8.0 min chromatography. 10-80AB_8 min.1 cm, purity 99.8%. LCMS Rₜ=2.181 min in 4.0 min chromatography, purity 99.8%, MS ESI calcd, for 566.24 [M+H]⁺ 567.24, found 567.1 Isomer 2. Compound 126 [(1S)-1-[3-[4-[2-(2-amino-3-pyridyl)-5-phenyl-imidazo[4,5-b]pyridin-3-yl]phenyl]azetidin-1-yl]ethyl]benzoic acid 126 (32.0 mg, 58% yield) as a yellow solid. ¹H NMR (400 MHz, DMSO-d₆) δ=12.18-12.02 (m, 1H), 8.36 (d, J=8.4 Hz, 1H), 8.16-8.11 (m, 1H), 8.09-7.98 (m, 5H), 7.92-7.73 (m, 4H), 7.68-7.58 (m, 4H), 7.52-7.40 (m, 3H), 6.93-6.85 (m, 1H), 4.92-4.78 (m, 1H), 4.63-4.52 (m, 1H), 4.47-4.34 (m, 1H), 4.19-4.00 (m, 3H), 1.58-1.50 (m, 3H). HPLC R; =2.607 min in 8.0 min chromatography, 10-80AB_8 min.1 cm, purity 99.246%. LCMS Rₜ=2.176 min in 4.0 min chromatography, purity 100.000%, MS ESI calcd, for 566.24 [M+H]⁺ 567.24, found 567.1.

Compounds Prepared in Accordance with General Scheme 1

The compounds herein (such as a compound of Formula I, or other compounds in Section I) may be prepared according to General Scheme 1, for example, from an intermediate as prepared in “Synthesis of additional intermediates for Table 3 compounds.” an intermediate that is commercially available, an intermediate as prepared elsewhere in this application or an intermediate that can be synthesized via a method known to those skilled in the art. An exemplary full synthesis is provided in Example 126 (Compound 126). Compounds prepared in accordance with General Scheme 1 are provided in Table 3 below.

TABLE 3

| | Compound | | | |
|---|---|---|---|---|
| Example | No. | Structure | 1H NMR | LCMS |
| 127 | 159 | 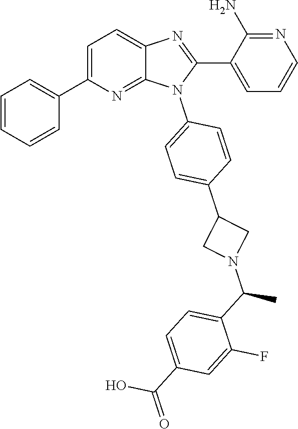 [SMILES: C[C@@H](c1ccc(C(=O)O)cc1F)N1CC(c2ccc(-n3c(-c4cccnc4N)nc4ccc(-c5ccccc5)nc43)cc2)C1] | ¹H NMR (400 MHz, DMSO-d₆) δ = 8.26 (d, J = 8.4 Hz, 1H), 8.05- 7.96 (m, 4H), 7.77 (dd, J = 1.2, 8.0 Hz, 1H), 7.68- 7.58 (m, 2H), 7.57-7.52 (m, 2H), 7.50-7.43 (m, 4H), 7.42-7.36 (m, 1H), 7.20 (dd, J = 1.6, 7.6 Hz, 1H), 6.94 (s, 2H), 6.42 (dd, J = 4.8, 7.6 Hz, 1H), 3.82 (q, J = 6.4 Hz, 1H), 3.73-3.68 (m, 2H), 3.58 (dd, J = 6.0, 8.8 Hz. 2H), 3.08 (t, J = 6.0 Hz, 1H), 1.18 (d, J = 6.4 Hz, 3H). ¹⁹F NMR (376 MHz, DMSO-d₆) δ = −119.09 | MS ESI caled. for 584.23, [M + H]⁻ 585.23, found 585.2 |
| 128 | 160 | 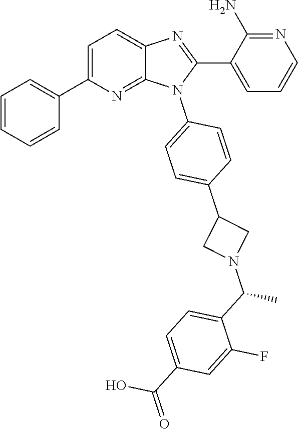 [SMILES: C[C@H](c1ccc(C(=O)O)cc1F)N1CC(c2ccc(-n3c(-c4cccnc4N)nc4ccc(-c5ccccc5)nc43)cc2)C1] | ¹H NMR (400 MHz, DMSO-d₆) δ = 8.25 (d, J = 8.8 Hz, 1H), 8.03- 7.95 (m, 4H), 7.78 (dd, J = 1.6, 8.0 Hz, 1H), 7.69- 7.60 (m, 2H), 7.56-7.51 (m, 2H), 7.48-7.42 (m, 4H), 7.42-7.36 (m, 1H), 7.20 (dd, J = 1.6, 7.6 Hz, 1H), 6.42 (dd, J = 4.8, 7.6 Hz, 1H), 3.92-3.83 (m, 1H), 3.75-3.69 (m, 2H), 3.61 (s, 1H), 3.30 (d, J = 5.2 Hz, 1H), 3.12 (d, J = 4.4 Hz, 1H), 1.20 (d. J = 6.4 Hz, 3H). ¹⁹F NMR (376 MHz, DMSO-d₆) δ = −73.52 (s, 1F). | MS ESI calcd. for 584.23, [M + H]⁻ 585.23, found 585.1 |
| 129 | 161 | 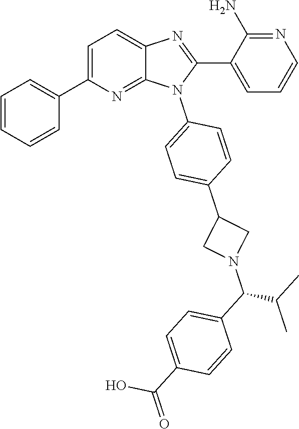 [SMILES: CC(C)[C@H](c1ccc(C(=O)O)cc1)N1CC(c2ccc(-n3c(-c4cccnc4N)nc4ccc(-c5ccccc5)nc43)cc2)C1] | ^{I}H NMR (400 MHz, DMSO-d₆) δ = 8.26 (d, J = 8.4 Hz, 1H). 8.04- 7.95 (m, 4H), 7.89 (d, J = 8.4 Hz, 2H), 7.55- 7.35 (m, 9H), 7.19 (dd, J = 1.2, 7.2 Hz, 1H), 6.93 (s. 2H), 6.41 (dd, J = 4.8, 7.6 Hz, 1H), 3.75-3.65 (m, 2H), 3.55-3.47 (m, 2H), 3.16-3.12 (m. 1H), 3.00-2.95 (m, 1H), 2.03- 1.93 (m, 1H), 0.80- 0.65 (m. 6H). | MS ESI calcd. for 594.27, [M + H]⁻ 595.27, found 595.2. |
| 130 | 162 | 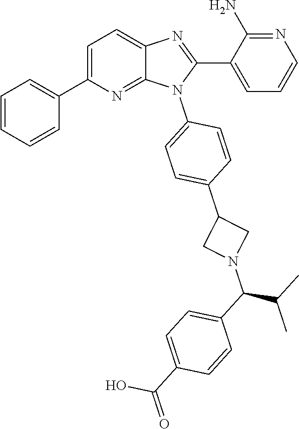 [SMILES: CC(C)[C@@H](c1ccc(C(=O)O)cc1)N1CC(c2ccc(-n3c(-c4cccnc4N)nc4ccc(-c5ccccc5)nc43)cc2)C1] | . ¹H NMR (400 MHz, DMSO-d₆) δ = 8.26 (d, J = 8.4 Hz, 1H), 8.04- 7.96 (m, 4H), 7.89 (d. J = 8.0 Hz, 2H), 7.56- 7.35 (m, 9H), 7.19 (dd, J = 1.6, 7.6 Hz, 1H), 6.93 (s, 2H), 6.41 (dd, J = 4.8, 7.6 Hz, 1H), 3.73-3.65 (m, 2H), 3.54-3.47 (m, 2H), 3.16-3.12 (m. 1H), 3.01-2.95 (m, 1H), 2.04- 1.90 (m, 1H), 0.80- 0.67 (m, 6H). | MS ESI caled. for 594.27, [M + H]⁻ 595.27, found 595.3. |
| 131 | 163 | 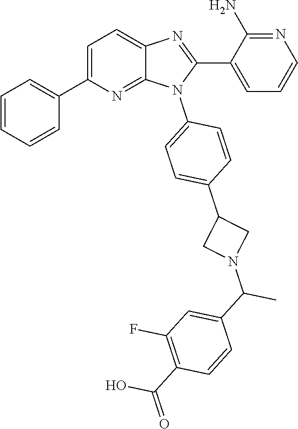 [SMILES: CC(c1ccc(C(=O)O)c(F)c1)N1CC(c2ccc(-n3c(-c4cccnc4N)nc4ccc(-c5ccccc5)nc43)cc2)C1] | . ¹H NMR (400 MHz, DMSO-d₆) δ = 8.26 (d, J = 8.4 Hz, 1H), 8.05- 7.95 (m, 4H), 7.80 (t, J = 8.0 Hz, 1H), 7.56-7.37 (m, 7H), 7.30-7.16 (m, 3H), 6.94 (s, 2H), 6.42 (dd, J = 4.8, 7.6 Hz, 1H), 3.76-3.68 (m, 2H), 3.55- 3.51 (m, 2H), 3.19- 3.17 (m, 1H), 3.10-3.04 (m, 1H), 1.16 (d, J = 6.4 Hz, 3H). | MS ESI calcd. for 584.23, [M + H]⁻ 585.23, found 585.2 |
| 132 | 164 | 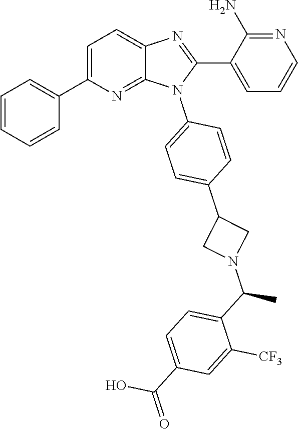 [SMILES: C[C@@H](c1ccc(C(=O)O)cc1C(F)(F)F)N1CC(c2ccc(-n3c(-c4cccnc4N)nc4ccc(-c5ccccc5)nc43)cc2)C1] | ¹H NMR (MeOD, 400 MHz,) δ = 8.31 (s, 1H), 8.28 (d, J = 8.0 Hz, 1H), 8.18 (d, J = 8.4 Hz, 1H), 8.05-8.00 (m, 2H), 8.00- 7.95 (m, 2H), 7.93 (d, J = 8.4 Hz, 1H), 7.56- 7.50 (m, 2H), 7.50-7.30 (m, 6H), 6.49 (dd. J = 5.2, 7.6 Hz, 1H), 4.20 (d, J = 5.6 Hz, 1H). 4.05- 3.97 (m. 1H), 3.88 (t. J = 7.6 Hz, 1H), 3.76 (t, J = 7.6 Hz, 1H), 3.59 (t, J = 7.6 Hz, 1H), 3.40 (t, J = 7.6 Hz, 1H), 1.37 (d, J = 6.4 Hz, 3H). | MS ESI calcd. for 634.24 [M + H]⁻ 635.24, found 635.2. |
| 133 | 165 | 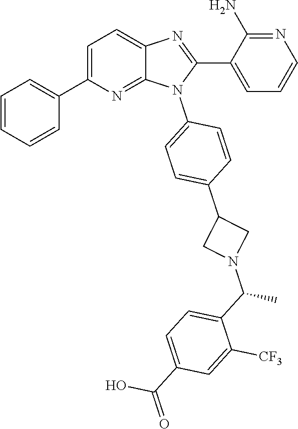 [SMILES: C[C@H](c1ccc(C(=O)O)cc1C(F)(F)F)N1CC(c2ccc(-n3c(-c4cccnc4N)nc4ccc(-c5ccccc5)nc43)cc2)C1] | ¹H NMR (MeOD, 400 MHz) δ = 8.32 (s, 1H), 8.28 (d. J = 8.4 Hz, 1H), 8.18 (d, J = 8.4 Hz, 1H), 8.05-8.00 (m, 2H), 8.00- 7.95 (m, 2H), 7.93 (d, J = 8.4 Hz, 1H), 7.56- 7.50 (m, 2H), 7.49-7.31 (m, 6H), 6.49 (dd. J = 5.2, 7.6 Hz, 1H), 4.26- 4.17 (m, 1H), 4.06-3.98 (m, 1H), 3.92-3.83 (m, 1H), 3.77 (t. J = 7.6 Hz, 1H), 3.60 (t, J = 7.6 Hz, 1H), 3.41 (t, J = 7.6 Hz, 1H), 1.38 (d, J = 6.4 Hz, 3H). | MS ESI calcd. for 634.24 [M + H]⁻ 635.24, found 635.2. |

### Example 134. Synthesis of 4-(1-(3-(4-(2-(2-aminopyridin-3-yl)-5-phenyl-3H-imidazo[4,5-b]pyridin-3-yl)-2-fluorophenyl) azetidin-1-yl)ethyl)benzoic acid (Compound 166)

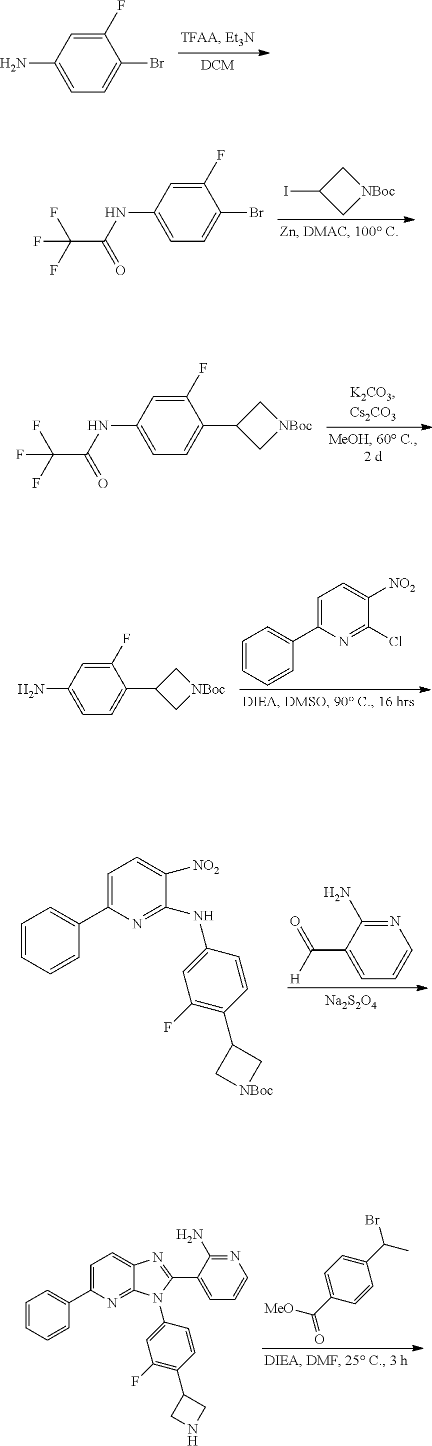

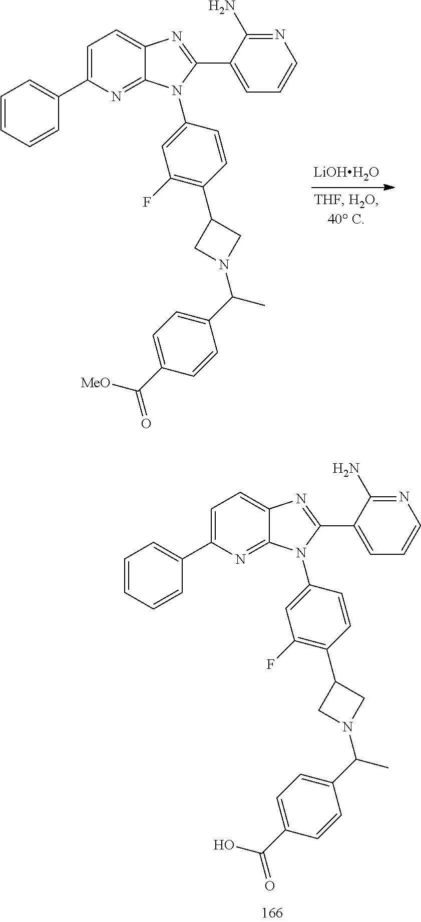 [SMILES: COC(=O)c1ccc(C(C)N2CC(c3ccc(-n4c(-c5cccnc5N)nc5ccc(-c6ccccc6)nc54)cc3F)C2)cc1 ; "LiOH·H2O" ; CC(c1ccc(C(=O)O)cc1)N1CC(c2ccc(-n3c(-c4cccnc4N)nc4ccc(-c5ccccc5)nc43)cc2F)C1]

Step 1. To a solution of 4-bromo-3-fluoro-aniline (9.00 g, 47.4 mmol), TEA (13 mL, 94.7 mmol) in DCM (50 mL) was added trifluoroacetic anhydride (11.94 g, 56.8 mmol) at 10° C. dropwise, then the mixture was stirred at 20° C. under N₂ for 12 hrs. The reaction mixture was poured into water (500 mL) and extracted with EtOAc (3×500 mL). The combined organic layer was washed with brine (500 mL), dried over Na₂SO₄, filtered and concentrated in vacuo to get N-(4-bromo-3-fluoro-phenyl)-2,2,2-trifluoro-acetamide (15.0 g, 72% yield) as a yellow solid. ¹H NMR (400 MHz, CHLOROFORM-d) δ=8.28 (s, 1H), 7.64 (dd, J=10.0, 2.4 Hz, 1H), 7.58 (dd, J=8.4, 7.6 Hz, 1H), 7.23 (dd, J=8.8, 1.6 Hz, 1H).

Step 2. A solution of N-(4-bromo-3~fluoro-phenyl)-2,2,2-trifluoro-acetamide (7.00 g. 24.5 mmol), tert-butyl 3-iodoazetidine-1-carboxylate (7.62 g. 26.9 mmol), Zn (9.39 g, 144 mmol), (1,2-dimethoxyethane)dichloronickel (II) (1.08 g, 4.89 mmol) and pyridine-2-carboxamidine hydrochloride (771 mg, 4.89 mmol) in DMA (70 mL) was stirred at 100° C. under N₂ for 4 hrs. The reaction was filtered and poured into water (200 mL) then extracted with EtOAc (5×500 mL). The combined organic layer was washed with brine (200 mL), dried over Na₂SO₄, filtered and concentrated in vacuo. The residue was purified by column chromatography (SiO₂, 20-25% EtOAc in PE) to get tert-butyl 3-[2-fluoro-4-[(2,2,2-trifluoroacetyl)amino]phenyl]azetidine-1-carboxylate (2.10 g, 24% yield) as a white solid. ¹H NMR (400 MHz, CHLOROFORM-d) δ=8.06 (s, 1H), 7.55 (dd, J=11.2, 2.0 Hz, 1H), 7.41 (s, 1H), 7.26 (d, J=2.0 Hz, 1H), 4.40-4.31 (m, 2H), 4.02 (d, J=7.2 Hz, 3H), 1.49 (s, 9H).

Step 3. A solution of tert-butyl 3-[2-fluoro-4-[(2,2,2-trifluoroacetyl)amino]phenyl]azetidine-1-carboxylate (2.00 g, 5.52 mmol), K₂CO₃ (2.29 g, 16.6 mmol) and Cs₂CO₃ (1.81 g, 5.58 mmol) in Methanol (20 mL) was stirred at 60° C. under N₂ for 12 hr. The reaction mixture was poured into water (100 mL) and extracted with EtOAc (3×150 mL). The combined organic layer was washed with brine (50 mL), dried over Na₂SO₄, filtered and concentrated in vacuo to get tert-butyl 3-(4-amino-2-fluoro-phenyl) azetidine-1-carboxylate (1.40 g, 73.3% yield) as yellow oil. ¹H NMR (400 MHz, CHLOROFORM-d) δ=7.14 (t, J=8.4 Hz, 1H), 6.52 (dd, J=7.6, 2.0 Hz, 1H), 6.43 (dd, J=11.6, 2.0 Hz, 1H), 4.33-4.25 (m, 2H), 3.99 (t, J=7.2 Hz, 2H), 3.93-3.83 (m, 1H), 1.48 (s, 9H).

Step 4. To a solution of tert-butyl 3-(4-amino-2-fluoro-phenyl) azetidine-1-carboxylate (1.36 g, 5.11 mmol) in DMSO (10 mL) was added 2-chloro-3-nitro-6-phenyl-pyridine (1.20 g, 5.11 mmol) and DIEA (4.2 mL, 25.6 mmol) under N₂. The mixture was stirred at 20° C. for 6 hrs at which point it was diluted with EtOAc (100 mL) and washed with 50 mL water then brine (3×50 m). The organic layer was collected and dried over Na₂SO₄ then concentrated in vacuo. The crude material was purified by flash column chromatography eluting with 30% of EtOAc in Isohexane to give tert-butyl 3-[2-fluoro-4-[(3-nitro-6-phenyl-2-pyridyl)amino]phenyl]azetidine-1-carboxylate (700 mg, 29% yield) as a red solid. ¹H NMR (400 MHz, DMSO-d₆) δ=10.20 (s, 1H), 8.68 (d, J=8.8 Hz, 1H), 8.21-8.09 (m, 2H), 7.84 (dd, J=12.8, 1.6 Hz, 1H), 7.69 (d, J=8.8 Hz, 1H), 7.64-7.59 (m, 3H), 7.57-7.49 (m, 1H), 4.30 (s, 2H), 4.11 (s, 1H), 4.08-3.94 (m, 3H), 1.45 (s, 9H).

Step 5. To a solution of tert-butyl 3-[2-fluoro-4-[(3-nitro-6-phenyl-2-pyridyl) amino]phenyl]azetidine-1-carboxylate (600 mg, 1.29 mmol) in DMSO (5 mL) and methanol (1 mL) was added 2-aminopyridine-3-carbaldehyde (316 mg, 2.58 mmol) and Na₂S₂O₄ (1.35 g. 7.75 mmol) at 25° C. The mixture was heated to 100° C. and stirred for 10 hrs then diluted with EtOAc (100 mL) and washed with water (50 mL) then brine (3×50 mL). The organic layer was collected and dried over Na₂SO₄ then concentrated in vacuo. The crude material was purified by flash column chromatography eluting with 100% of EtOAc in Isohexane and prep-HPLC (Column: Welch Xtimate C18 40*200 mm 7 μm; mobile phase: [water (FA)-ACN]; B %: 8%-48%; 25 min) to give tert-butyl 3-[4-[2-(2-amino-3-pyridyl)-5-phenyl-imidazo[4,5-b]pyridin-3-yl]-2-fluoro-phenyl]azetidine-1-carboxylate (3.00 g, crude) as a yellow solid. ¹H NMR (400 MHz, DMSO-d₆) δ=8.43-8.35 (m, 2H), 8.32 (d, J=8.4 Hz, 1H), 8.11-8.02 (m, 4H), 7.75-7.68 (m, 1H), 7.53 (br t, J=8.0 Hz, 3H), 7.46 (d, J=7.6 Hz, 1H), 7.42-7.37 (m, 1H), 7.35-7.31 (m, 1H), 6.87 (s, 1H), 6.53 (dd, J=7.6, 4.8 Hz, 1H), 4.36-4.24 (m, 1H), 4.11-3.99 (m, 4H).

Step 6. To a solution of 3-[3-[4-(azetidin-3-yl)-3-fluoro-phenyl]-5-phenyl-imidazo [4,5-b]pyridin-2-yl]pyridin-2-amine (110 mg, 0.252 mmol) in DMF (10 mL) was added DIEA (0.13 mL, 0.756 mmol) and methyl 4-(1-bromoethyl)benzoate (67 mg, 0.277 mmol). The mixture was stirred at 25° C. for 16 hrs. The reaction mixture was diluted with H₂O (3 mL) and filtered. The filtered pad was washed with ACN (5 mL) then concentrated in vacuo to give methyl 4-[1-[3-[4-[2-(2-amino-3-pyridyl)-5-phenyl-imidazo[4,5-b]pyridin-3-yl]-2-fluoro-phenyl]azetidin-1-yl]ethyl]benzoate (80.0 mg, 53% yield) as a white solid. ¹H NMR (400 MHz, DMSO-d₆) δ=8.28 (d, J=8.4 Hz, 1H), 8.08-7.89 (m, 9H), 7.61 (t, J=8.0 Hz, 1H), 7.53-7.39 (m, 6H), 7.33-7.27 (m, 2H), 6.83 (s, 2H), 6.49 (dd, J=7.6, 4.8 Hz, 1H), 3.85 (s, 3H), 3.54-3.44 (m, 3H), 3.21 (s, 2H), 3.08 (s, 1H).

Step 7. To a solution of methyl 4-[1-[3-[4-[2-(2-amino-3-pyridyl)-5-phenyl-imidazo[4,5-b]pyridin-3-yl]-2-fluoro-phenyl]azetidin-1-yl]ethyl]benzoate (80.0 mg, 0.134 mmol) in water (1 mL), THF (3 mL) was added LiOH·H₂O (28.0 mg, 0.668 mmol) under N₂ atmosphere. The mixture was heated to 40° C. and stirred for 2 hrs at which point the crude material was purified by prep-HPLC (column: Welch Xtimate C18 200*40 mm*7 μm; mobile phase: [water (FA)-ACN]: B %: 0%-38%, 25 min) to give 4-[1-[3-[4-[2-(2-amino-3-pyridyl)-5-phenyl-imidazo[4,5-b]pyridin-3-yl]-2-fluoro-phenyl]azetidin-1-yl]ethyl]benzoic acid 166 (18.0 mg, 22% yield) as a white solid. ¹H NMR (400 MHz, DMSO-d₆) δ=8.28 (d, J=8.4 Hz, 1H), 8.07-7.97 (m, 4H), 7.90 (d, J=8.0 Hz, 2H), 7.62 (t, J=8.4 Hz, 1H), 7.53-7.37 (m, 6H), 7.34-7.27 (m, 2H), 6.84 (s, 2H), 6.49 (dd, J=7.6, 4.8 Hz, 1H), 3.90-3.73 (m, 2H), 3.53-3.47 (m, 2H), 3.21 (s, 1H), 3.08 (t, J=7.2 Hz, 1H), 1.17 (d, J=6.4 Hz, 3H). HPLC Rₜ=2.441 min in 8 min chromatography, ACE Excel 3 C18 4.6*100 mm, purity 99.9%. LCMS Rₜ=1.584 min in 4 min chromatography, Agilent PoroShell 120 EC-C18 2.7 μm 3.0*50 mm, purity 99.5%, MS ESI calcd, for 584.23 [M+H]⁺ 585.23, found 585.2.

### Example 135. Synthesis of 4-(1-(3-(5-(2-(2-aminopyridin-3-yl)-5-phenyl-3H-imidazo[4,5-b]pyridin-3-yl)pyridin-2-yl) azetidin-1-yl)ethyl)benzoic acid (Compound 167)

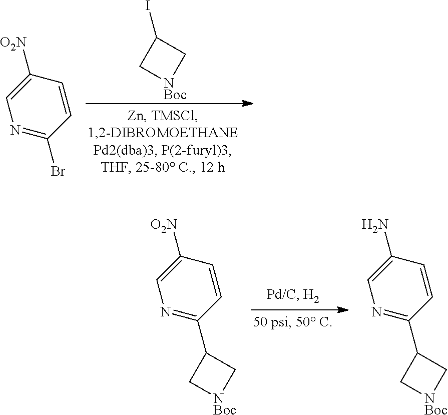 [SMILES: O=[N+]([O-])c1ccc(Br)nc1 ; CC(C)(C)OC(=O)N1CC(I)C1 ; CC(C)(C)OC(=O)N1CC(c2ccc([N+](=O)[O-])cn2)C1 ; CC(C)(C)OC(=O)N1CC(c2ccc(N)cn2)C1]

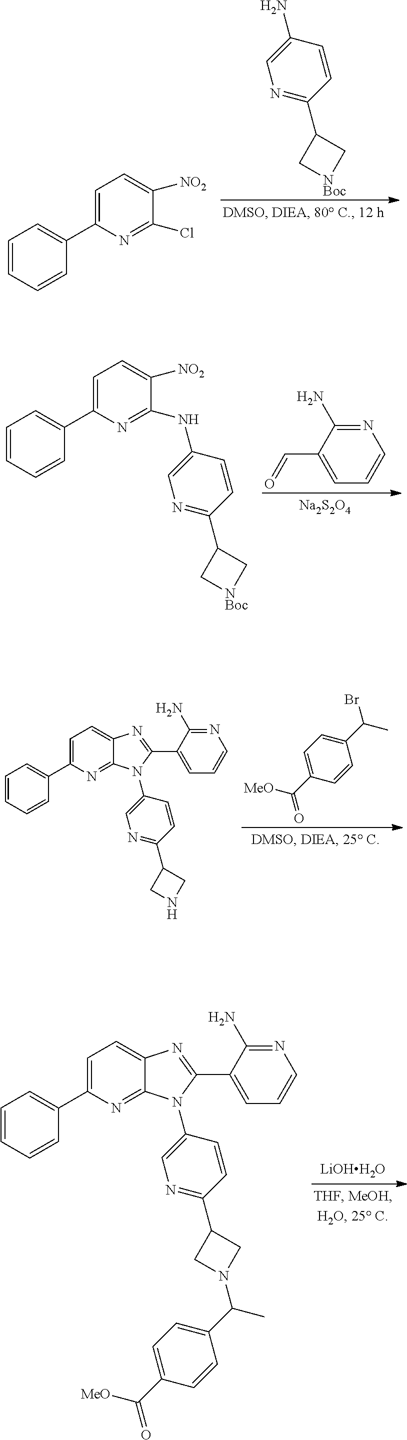

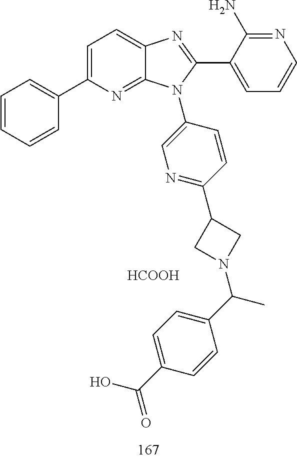 [SMILES: CC(c1ccc(C(=O)O)cc1)N1CC(c2ccc(-n3c(-c4cccnc4N)nc4ccc(-c5ccccc5)nc43)cn2)C1 ; "HCOOH"]

Step 1. Zn (3.49 g, 53.7 mmol) was vigorously stirred in THF (24 mL) under nitrogen and 1,2-DIBROMOETHANE (1.17 g, 6.20 mmol) was added. The suspension was then heated at 80° C. for 8 mins and next allowed to cool to room temperature. TMSCl (673 mg, 6.20 mmol) in THF (12 mL) was then added and the mixture further stirred at room temperature for 45 mins. A solution of tert-butyl 3-iodoazetidine-1-carboxylate (11.7 g, 41.3 mmol) in THF (24 mL) was then added dropwise to the solution over a period of 15 mins and the reaction mixture stirred at room temperature for 2 hrs. Pd₂(dba)₃ (757 mg, 0.827 mmol) and P (2-furyl) 3 (576 mg, 2.48 mmol) were then added to the mixture, followed by 2-bromo-5-nitro-pyridine (9.20 g, 45.5 mmol) in THF (48 mL). The mixture was then heated at 55° C. for 3 hrs, cooled to room temperature and quenched with brine (200 mL) the product was extracted with DCM (3×300 mL). The combined organic layers were dried over Na₂SO₄ then concentrated in vacuo. The crude was purified by flash column chromatography (heptane:ethyl acetate=3:1) to give tert-butyl 3-(5-nitro-2-pyridyl) azetidine-1-carboxylate (8.62 g. 75% yield) as brown oil. ¹H NMR (400 MHz, DMSO-d₆) δ=9.39 (d, J=2.4 Hz, 1H), 8.55 (dd, J=8.4, 2.4 Hz, 1H), 7.64 (d, J=8.4 Hz, 1H), 4.22 (d, J=7.2 Hz, 2H), 4.11 (dd, J=8.4, 5.6 Hz, 1H), 4.06-4.00 (m, 2H), 1.39 (s, 9H).

Step 2. To a solution of tert-butyl 3-(5-nitro-2-pyridyl) azetidine-1-carboxylate (8.60 g, 30.8 mmol) in THF (80 mL) was added wet Pd/C (10%, 2.00 g) under N₂ atmosphere. The suspension was degassed and purged with H₂ for 3 times. The reaction mixture was stirred under H₂ (50 Psi) at 50° C. for 16 hrs. The mixture was filtered via a celite pad and the filter cake was washed with DCM:MeOH=1:1 (300 mL). The organic phase was concentrated to give tert-butyl 3-(5-amino-2-pyridyl) azetidine-1-carboxylate (6.20 g, crude) as yellow oil. ¹H NMR (400 MHz, DMSO-d₆) δ=7.99 (d, J=2.4 Hz, 1H), 7.01-6.95 (m, 1H), 6.92-6.88 (m, 1H), 5.25 (br s, 1H), 4.12 (s, 1H), 3.94 (s, 2H), 3.79-3.66 (m, 1H), 1.43 (s, 9H).

Step 3. To a solution of tert-butyl 3-(5-amino-2-pyridyl) azetidine-1-carboxylate (5.70 g, 22.9 mmol) in DMSO (60 mL) was added 2-chloro-3-nitro-6-phenyl-pyridine (6.40 g, 27.3 mmol), DIEA (19 mL, 114 mmol) under N₂ atmosphere. The mixture was heated to 100° C. and stirred for 6 hrs. The reaction was diluted with EtOAc (500 mL) and washed with water (100 mL) then brine (3×100 mL). The organic layer was collected and dried over Na₂SO₄ then concentrated in vacuo. The resulting crude material was purified by flash column chromatography eluting with 30% of EtOAc in Isohexane to give tert-butyl 3-[5-[(3-nitro-6-phenyl-2-pyridyl)amino]-2-pyridyl]azetidine-1-carboxylate (6.20 g, 55% yield) as a red solid. ¹H NMR (400 MHz, DMSO-d₆) δ=10.14 (s, 1H), 8.92 (d, J=2.4 Hz, 1H), 8.64 (d, J=8.4 Hz. 1H), 8.15 (dd, J=8.4, 2.4 Hz, 1H), 8.10-8.01 (m, 2H), 7.63 (d, J=8.4 Hz, 1H), 7.56-7.51 (m, 3H), 7.41 (d, J=8.4 Hz, 1H), 4.28-4.15 (m, 2H), 4.08-4.00 (m, 2H), 3.99-3.90 (m, 1H), 1.42 (s, 9H).

Step 4. To a solution of tert-butyl 3-[5-[(3-nitro-6-phenyl-2-pyridyl)amino]-2-pyridyl]azetidine-1-carboxylate (5.80 g, 13.0 mmol) in DMSO (50 mL) and Methanol (10 mL) was added 2-aminopyridine-3-carbaldehyde (3.16 g, 25.9 mmol) and Na₂S₂O₄ (13.5 g, 77.8 mmol) at 25° C. The mixture was heated to 100° C. and stirred for 10 hrs at which point the crude product was purified by prep-HPLC (column: Phenomenex luna C18 250*80 mm*10 μm; mobile phase: [water (FA)-ACN]; B %: 0%-30%, 15 min) to give 3-[3-[6-(azetidin-3-yl)-3-pyridyl]-5-phenyl-imidazo[4,5-b]pyridin-2-yl]pyridin-2-amine (2.50 g, 46% yield) as a brown solid. ¹H NMR (400 MHz, DMSO-d₆) δ=8.78 (d, J=2.0 Hz, 1H), 8.35 (s, 1H), 8.30 (d, J=8.4 Hz, 1H), 8.06-8.00 (m, 4H), 7.96 (dd, J=8.4, 2.4 Hz, 1H), 7.57 (d, J=8.0 Hz, 1H), 7.50-7.45 (m, 2H), 7.43-7.38 (m, 1H), 7.35 (dd, J=7.6, 1.6 Hz, 1H), 6.72 (s, 2H), 6.50 (dd, J=7.6, 4.8 Hz, 1H), 4.27 (br d, J=8.4 Hz, 1H), 4.19-4.14 (m, 4H).

Step 5. To a solution of 3-[3-[6-(azetidin-3-yl)-3-pyridyl]-5-phenyl-imidazo[4,5-b]pyridin-2-yl]pyridin-2-amine (220 mg, 0.524 mmol) in DMSO (3 mL) was added DIEA (0.47 mL, 2.62 mmol) and methyl 4-(1-bromoethyl)benzoate (191 mg, 0.787 mmol). The mixture was heated to 80° C. and stirred for 16 hrs at which point it was diluted with EtOAc (100 mL) and the resulting mixture was washed with water (50 mL) then brine (3×50 mL). The organic layer was collected and dried over Na₂SO₄ then concentrated in vacuo giving methyl 4-[1-[3-[5-[2-(2-amino-3-pyridyl)-5-phenyl-imidazo[4,5-b]pyridin-3-yl]-2-pyridyl]azetidin-1-yl]ethyl]benzoate (310 mg, crude) as a white solid. ¹H NMR (400 MHz, DMSO-d₆) δ=8.67 (d, J=2.4 Hz, 1H), 8.37-8.23 (m, 2H), 8.08 (s, 2H), 8.06-8.01 (m, 3H), 7.96 (d, J=8.4 Hz, 2H), 7.92 (d, J=8.4 Hz, 2H), 7.60-7.30 (m, 8H), 6.72 (s, 2H), 6.50 (dd, J=4.8, 7.6 Hz, 1H), 5.97 (d, J=6.4 Hz, 1H), 3.85 (s, 3H), 2.63 (s, 1H), 1.52 (d, J=6.4 Hz, 3H).

Step 6. To a solution of methyl 4-[1-[3-[5-[2-(2-amino-3-pyridyl)-5-phenyl-imidazo[4,5-b]pyridin-3-yl]-2-pyridyl]azetidin-1-yl]ethyl]benzoate (310 mg, 0.533 mmol) in water (1 mL) and THF (3 mL) was added LiOH·H₂O (112 mg, 2.66 mmol) under N₂ atmosphere. The mixture was stirred at 40° C. for 2 hrs. The crude product was purified by prep-HPLC (column: Welch Xtimate C18 200*40 mm*7 μm; mobile phase: [water (FA)-ACN]; B %: 0%-36%, 20 min4-[1-[3-[5-[2-(2-amino-3-pyridyl)-5-phenyl-imidazo[4,5-b]pyridin-3-yl]-2-pyridyl]azetidin-1-yl]ethyl]benzoic acid 167 (111 mg, 36% yield) as a white solid. ¹H NMR (400 MHz, DMSO-d₆) δ=8.67 (d, J=2.4 Hz, 1H), 8.29 (d, J=8.4 Hz, 1H), 8.14 (s, 1H), 8.07-7.99 (m, 4H), 7.92-7.86 (m, 3H), 7.57 (d, J=8.4 Hz, 1H), 7.51-7.44 (m, 4H), 7.41 (d, J=7.2 Hz, 1H), 7.33 (dd, J=1.6, 7.6 Hz, 1H), 6.73 (s, 2H), 6.49 (dd, J=4.8, 7.6 Hz, 1H), 3.82 (d, J=7.2 Hz, 1H), 3.73 (s, 1H), 3.52 (d, J=6.4 Hz, 1H), 3.45 (s, 1H), 3.37-3.36 (m, 1H), 3.20 (s, 1H), 1.17 (d, J=6.4 Hz, 3H). HPLC Rₜ=2.031 in 8 min chromatography, ACE Excel 3 C18 4.6*100 mm, purity 99% LCMS Rₜ=1.448 min in 4 min chromatography, Agilent PoroShell 120 EC-C18 2.7 μm 3.0*50 mm, purity 98.9%, MS ESI calcd, for 567.24 [M+H]⁺ 568.24, found 568.3.

### Example 136. Synthesis of 4-((5-(2-(2-aminopyridin-3-yl)-6-phenyl-1H-benzo[d]imidazol-1-yl)-6-methylpyridin-2-yl)carbamoyl)benzoic acid (Compound 168)

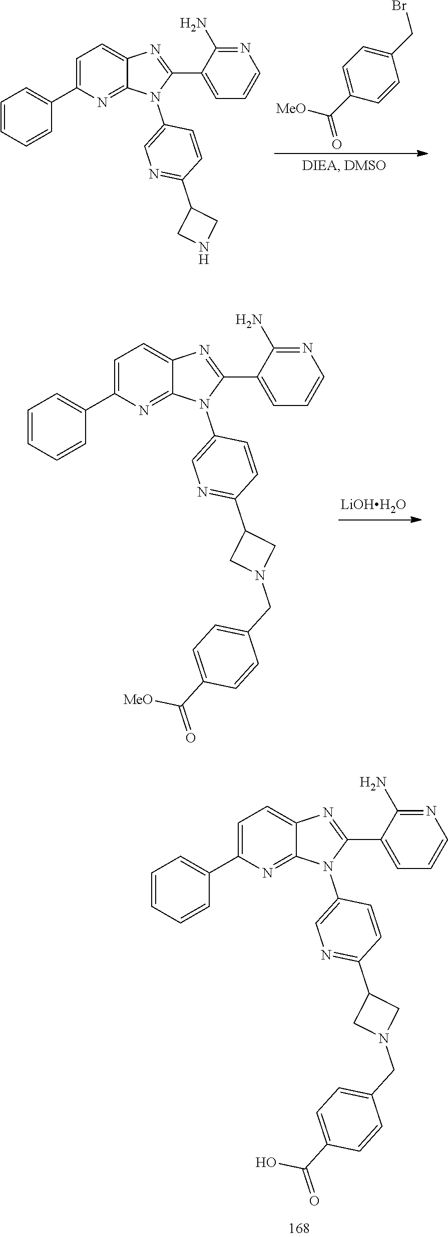 [SMILES: Nc1ncccc1-c1nc2ccc(-c3ccccc3)nc2n1-c1ccc(C2CNC2)nc1 ; COC(=O)c1ccc(CBr)cc1 ; COC(=O)c1ccc(CN2CC(c3ccc(-n4c(-c5cccnc5N)nc5ccc(-c6ccccc6)nc54)cn3)C2)cc1 ; "LiOH·H2O" ; Nc1ncccc1-c1nc2ccc(-c3ccccc3)nc2n1-c1ccc(C2CN(Cc3ccc(C(=O)O)cc3)C2)nc1]

Step 1. To a solution of 3-[3-[6-(azetidin-3-yl)-3-pyridyl]-5-phenyl-imidazo[4,5-b]pyridin-2-yl]pyridin-2-amine (220 mg, 0.524 mmol) in DMSO (3 mL) was added DIEA (0.47 mL, 2.62 mmol) and methyl 4-(bromomethyl)benzoate (180 mg, 0.787 mmol). The mixture was stirred heated to 80° C. and stirred for 16 hrs. The reaction was then diluted with EtOAc (50 mL) and washed with water (50 mL) then brine (3×50 mL). The organic layer was then collected and dried over Na₂SO₄ then concentrated in vacuo. The crude was then purified by flash column chromatography eluting with 30% of MeOH in DCM to give methyl 4-[[3-[5-[2-(2-amino-3-pyridyl)-5-phenyl-imidazo[4,5-b]pyridin-3-yl]-2-pyridyl]azetidin-1-yl]methyl]benzoate (170 mg, 57% yield) as a white solid. ¹H NMR (400 MHz, DMSO-d₆) δ=8.29 (d, J=8.4 Hz, 1H), 8.06-7.99 (m, 5H), 7.92 (d, J=7.6 Hz, 3H), 7.58 (d, J=8.4 Hz, 1H), 7.48 (t, J=7.6 Hz, 3H), 7.41 (d, J=7.2 Hz, 1H), 7.36-7.29 (m, 2H), 6.72 (s, 2H), 6.49 (dd, J=7.6, 4.8 Hz, 1H), 3.84 (s, 3H), 3.79-3.69 (m, 3H), 3.65-3.56 (m, 2H), 3.16 (d, J=5.2 Hz, 2H).

Step 2. To a solution of methyl 4-[[3-[5-[2-(2-amino-3-pyridyl)-5-phenyl-imidazo[4,5-b]pyridin-3-yl]-2-pyridyl]azetidin-1-yl]methyl]benzoate (250 mg, 0.440 mmol) in Water (1 mL) and THF (3 mL) was added LiOH·H₂O (92.0 mg, 2.20 mmol) under N₂ atmosphere. The mixture was heated to 40° C. and stirred for 2 hrs. The crude product was purified by prep-HPLC (column: Welch Xtimate C18 200*40 mm*7 μm; mobile phase: [water (FA)-ACN]; B %: 0%-34%, 20 min). 4-[[3-[5-[2-(2-amino-3-pyridyl)-5-phenyl-imidazo[4,5-b]pyridin-3-yl]-2-pyridyl]azetidin-1-yl]methyl]benzoic acid 168 (55.0 mg, 22% yield) as a white solid. ¹H NMR (400 MHz, DMSO-d₆) δ=8.68 (d, J=2.0 Hz, 1H), 8.29 (d, J=8.4 Hz, 1H), 8.07-7.98 (m, 4H), 7.86-7.93 (m, 3H), 7.58 (d, J=8.0 Hz, 1H), 7.51-7.38 (m, 5H), 7.33 (dd, J=7.6, 1.6 Hz, 1H), 6.73 (s, 2H), 6.49 (dd, J=7.6, 4.8 Hz, 1H), 3.90 (t, J=7.6 Hz, 1H), 3.72 (s, 2H), 3.67 (t, J=7.2 Hz, 2H), 3.40 (s, 2H). HPLC Rₜ=2.141 min in 8 min chromatography, ACE Excel 3 C18 4.6*100 mm, purity 99%. LCMS Rₜ=1.450 min in 4 min chromatography, Agilent PoroShell 120 EC-C18 2.7 μm 3.0*50 mm, purity 99.0%, MS ESI calcd, for 553.22 [M+H]⁺ 554.22, found 554.3.

### Example 137. Synthesis of 4-(1-(4-(4-(2-(2-aminopyridin-3-yl)-5-phenyl-3H-imidazo[4,5-b]pyridin-3-yl)phenyl)piperidin-1-yl)ethyl)benzoic acid (Compound 169)

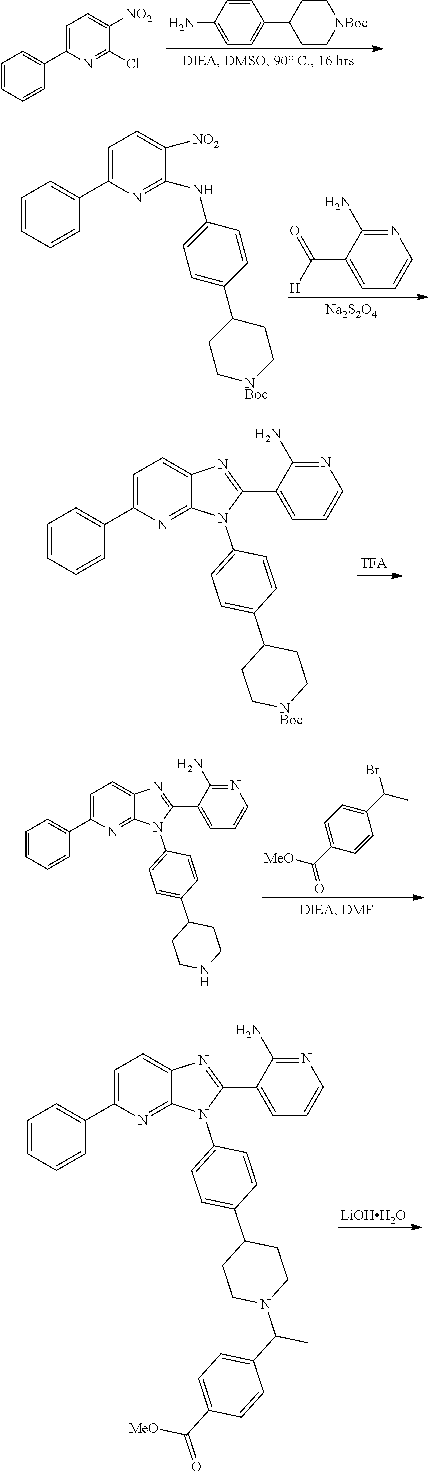

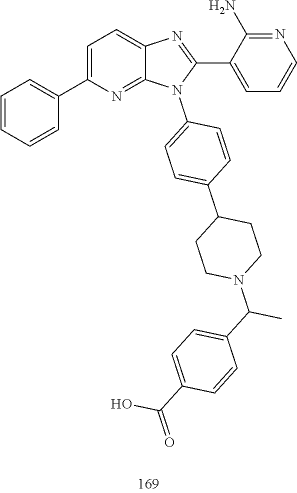 [SMILES: CC(c1ccc(C(=O)O)cc1)N1CCC(c2ccc(-n3c(-c4cccnc4N)nc4ccc(-c5ccccc5)nc43)cc2)CC1]

Step 1. To a solution of tert-butyl 4-(4-aminophenyl)piperidine-1-carboxylate (2.83 g, 10.2 mmol) in DMSO (20 mL) was added DIEA (3.30 g, 25.6 mmol) and 2-chloro-3-nitro-6-phenylpyridine (2.00 g, 8.52 mmol) at 25° C. The mixture was heated to 80° C. for and stirred for 14 hrs. at which point EtOAc (50 mL) was added to the reaction. The resulting suspension was washed with brine (3×40 mL), dried over anhydrous Na₂SO₄, filtered and concentrated in vacuo. The crude product was triturated with ACN (50 mL). The reaction mixture was filtered. The filter cake was concentrated to give tert-butyl 4-(4-((3-nitro-6-phenylpyridin-2-yl)amino)phenyl)piperidine-1-carboxylate (3.30 g, 82% yield) as a red solid. ¹H NMR (400 MHz, DMSO-d₆) δ=10.09 (s, 1H), 8.60 (d, J=8.8 Hz, 1H), 8.13-8.06 (m, 2H), 7.69 (d, J=8.4 Hz, 2H), 7.61-7.51 (m, 4H), 7.30 (d, J=8.4 Hz, 2H), 4.09 (d, J=10.4 Hz, 2H), 3.33-3.32 (m, 2H), 2.74-2.65 (m, 1H), 1.83-1.75 (m, 2H), 1.57-1.47 (m, 2H), 1.42 (s, 9H).

Step 2. To a solution of tert-butyl 4-(4-((3-nitro-6-phenylpyridin-2-yl)amino)phenyl)piperidine-1-carboxylate (2.80 g, 5.90 mmol) and 2-aminonicotinaldehyde (1.44 g, 11.8 mmol) in DMSO (28 mL) and Methanol (28 mL) was added Na₂S₂O₄ (5.96 g, 34.2 mmol). The mixture was heated to 100° C. and stirred for 12 hrs. The reaction mixture was concentrated and taken up in Water (40 mL). The resulting mixture was extracted with EtOAc (3×60 mL) and the combined organic phase was washed with brine (50 mL), dried over anbydrous Na₂SO₄, filtered and concentrated in vacuo. The residue was purified by flash silica gel chromatography (eluent of 0~52% EtOAc in PE) to give tert-butyl 4-(4-(2-(2-aminopyridin-3-yl)-5-phenyl-3H-imidazo[4,5-b]pyridin-3-yl)phenyl)piperidine-1-carboxylate (1.23 g, 38% yield) as a yellow solid. ¹H NMR (400 MHz, DMSO-d₆) δ=8.26 (d, J=8.4 Hz, 1H), 8.07-7.90 (m, 4H), 7.54-7.37 (m, 7H), 7.16 (dd, J=1.6, 7.6 Hz, 1H), 6.95 (s, 2H), 6.40 (dd, J=4.4, 7.6 Hz, 1H), 4.13-4.05 (m, 2H), 2.91-2.80 (m, 3H), 1.84 (d, J=11.6 Hz, 2H), 1.54 (dd, J=4.0, 12.4 Hz, 2H), 1.42 (s, 9H).

Step 3. To a solution of tert-butyl 4-(4-(2-(2-aminopyridin-3-yl)-5-phenyl-3H-imidazo[4,5-b]pyridin-3-yl)phenyl)piperidine-1-carboxylate (230 mg, 0.421 mmol) in 1,4-Dioxane (2 mL) was added HCl/dioxane (15 mL, 2M). The mixture was stirred at 25° C. for 12 hrs. The reaction mixture was concentrated to give 3-(5-phenyl-3-(4-(piperidin-4-yl)phenyl)-3H-imidazo[4,5-b]pyridin-2-yl)pyridin-2-amine (190 mg, crude, TFA salt) as a yellow solid. ¹H NMR (400 MHz, DMSO-ds) 8=8.90 (s, 2H), 8.35 (d, J=8.4 Hz, 1H), 8.14-8.00 (m, 4H), 7.78 (d, J=7.6 Hz, 1H), 7.60-7.42 (m, 7H), 6.83 (t, J=6.4 Hz, 1H), 3.28-3.22 (m, 1H), 3.10-2.91 (m, 4H), 2.08-1.81 (m, 5H).

Step 4. To a solution of 3-(5-phenyl-3-(4-(piperidin-4-yl)phenyl)-3H-imidazo[4,5-b]pyridin-2-yl)pyridin-2-amine (190 mg, 0.425 mmol) in DMSO (3.00 mL) was added DIEA (160 mg, 1.28 mmol) and methyl 4-(1-bromoethyl)benzoate (120 mg, 0.511 mmol) at 25° C.

The mixture was heated to 80° C. and stirred for 14 hrs. at which point EtOAc (10 mL) was added. The resulting suspension was washed with brine (3×20 mL), dried over anhydrous Na₂SO₄, filtered and concentrated. The residue was purified by flash silica gel chromatography (eluent of 0~10% MeOH in DCM) to give methyl 4-(1-(4-(4-(2-(2-aminopyridin-3-yl)-5-phenyl-3H-imidazo[4,5-b]pyridin-3-yl)phenyl)piperidin-1-yl)ethyl)benzoate (250 mg, 97% yield) as a brown solid. LCMS Rₜ=0.517 min in 1 min chromatography, purity 73.8%, MS ESI calcd, for 608.29. [M+H]⁺ 609.29, found 609.4.

Step 5. To a solution of methyl 4-(1-(4-(4-(2-(2-aminopyridin-3-yl)-5-phenyl-3H-imidazo[4,5-b]pyridin-3-yl)phenyl)piperidin-1-yl)ethyl)benzoate (120 mg, 0.197 mmol) in THF (2 mL) and water (1 mL) was added LiOH·H₂O (35 mg, 0.838 mmol). The mixture was stirred at 25° C. for 1 hrs. Methanol (0.5 mL) was added to the mixture. The mixture was stirred at 25° C. for 1 hr. The mixture was filtered and the residue was purified by prep-HPLC (Column: Welch Xtimate C18 40*200 mm 7 μm; mobile phase: water (NH₄HCO₃)-ACN; B %: 12%-52%; 25 min) to give 4-(1-(4-(4-(2-(2-aminopyridin-3-yl)-5-phenyl-3H-imidazo[4,5-b]pyridin-3-yl)phenyl)piperidin-1-yl)ethyl)benzoic acid 169 (20.0 mg, 17% yield) as a white solid. ¹H NMR (DMSO-d₆, 400 MHz) δ_{H}=8.26 (d, J=8.0 Hz, 1H), 8.05-7.87 (m, 6H), 7.51-7.37 (m, 9H), 7.16 (dd, J=2.0, 7.6 Hz, 1H), 6.95 (s, 2H), 6.39 (dd, J=4.8, 7.6 Hz, 1H), 3.60 (q. J=6.4 Hz, 1H), 3.13-3.05 (m, 1H), 2.86 (d, J=11.2 Hz, 1H), 2.60-2.52 (m, 3H), 2.13-1.94 (m, 2H), 1.82-1.68 (m, 2H), 1.34 (d, J=6.8 Hz, 3H). HPLC Rₜ=3.853 min in 8 min chromatography, purity 98%. LCMS Rₜ=2.212 min in 4 min chromatography, purity 98.27%, MS ESI calcd, for 594.27, [M+H]⁺ 595.27, found 595.2.

### Example 138. Synthesis of 4-(1-(3-(4-(2-(2-aminopyridin-3-yl)-5-phenyl-3H-imidazo[4,5-b]pyridin-3-yl)phenyl)piperidin-1-yl)ethyl)benzoic acid (Compound 170)

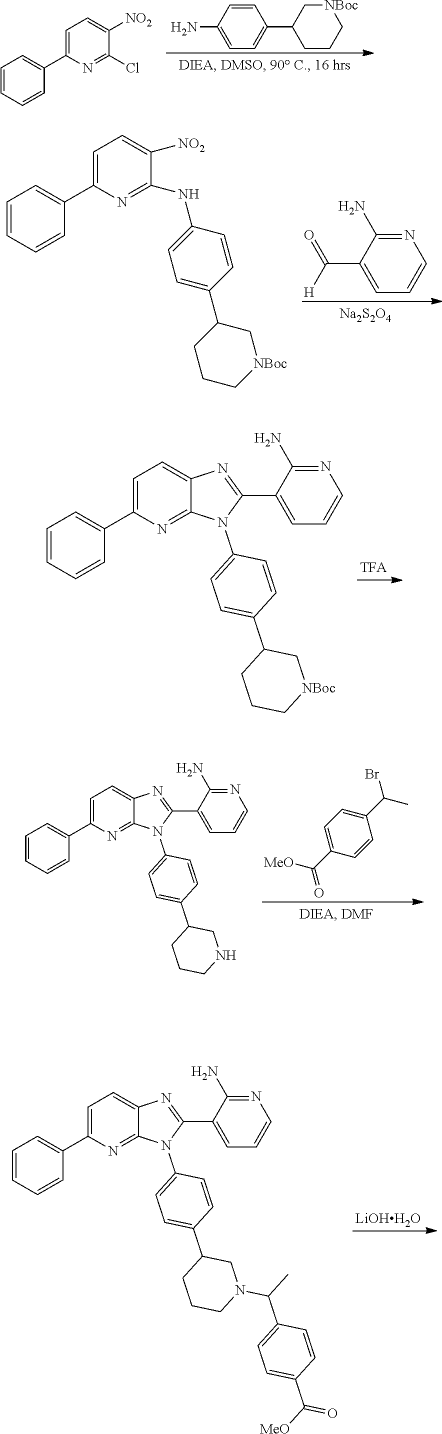

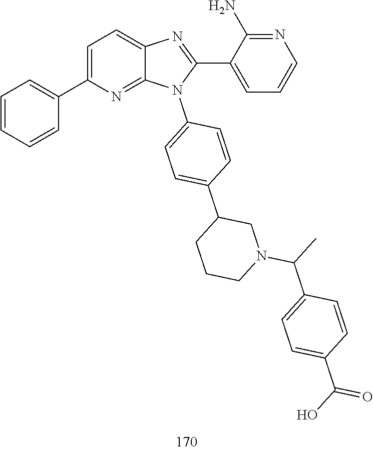 [SMILES: CC(c1ccc(C(=O)O)cc1)N1CCCC(c2ccc(-n3c(-c4cccnc4N)nc4ccc(-c5ccccc5)nc43)cc2)C1]

Step 1. A mixture of 2-chloro-3-nitro-6-phenyl-pyridine (3.40 g, 14.5 mmol), tert-butyl 3-(4-aminophenyl)piperidine-1-carboxylate (4.41 g, 15.9 mmol) and DIEA (7.80 mL, 43.5 mmol) in DMSO (50 mL) was stirred at 90° C. for 16 hrs under N₂. Water (200 mL) was added to the mixture and the product was extracted with EtOAc (3×200 mL). The organic phase was washed with brine (3×50 mL), dried over Na₂SO₄ and concentrated. The residue was purified by column (EtOAc in PE, 0-30%) to give ter-butyl 3-[4-[(3-nitro-6-phenyl-2-pyridyl)amino]phenyl]piperidine-1-carboxylate (7.00 g, crude) as a red solid. ¹H NMR (CHLOROFORM-d 400 MHz) δ_{H}=10.30 (s, 1H), 8.60 (d, J=8.8 Hz, 1H), 8.11-8.01 (m, 2H), 7.73 (d, J=8.4 Hz, 2H), 7.56-7.47 (m, 3H), 7.31 (dd, J=3.2, 8.4 Hz, 3H), 4.33-4.09 (m, 2H), 2.87-2.66 (m, 3H), 2.14-2.00 (m, 1H), 1.84-1.75 (m, 1H), 1.59 (br s, 2H), 1.50 (s, 9H).

Step 2. To a solution of tert-butyl 3-[4-[(3-nitro-6-phenyl-2-pyridyl)amino]phenyl]piperidine-1-carboxylate (3.00 g. 6.32 mmol) and 2-aminopyridine-3-carbaldehyde (1.04 g, 8.53 mmol) in DMSO (30 mL) and methanol (3 mL) was added Na₂S₂O₄ (3.30 g, 19.0 mmol) under N₂. The mixture was stirred at 100° C. for 12 hrs. then concentrated and Saturated aq. LiCl (30 mL) was added. The resulting mixture was extracted with EtOAc (3×30 mL) and the combined organic phase was washed with brine (30 mL), dried over anhydrous Na₂SO₄, filtered and concentrated. The residue was purified by flash silica gel chromatography (eluent of 0~99% EtOAc in PE) to give tert-butyl 3-[4-[2-(2-amino-3-pyridyl)-5-phenyl-imidazo[4,5-b]pyridin-3-yl]phenyl]piperidine-1-carboxylate (2.00 g, 58% yield) as a yellow solid. ¹H NMR (DMSO-d₆, 400 MHz) δ_{H}=8.26 (d, J=8.4 Hz, 1H), 8.08-7.92 (m, 4H), 7.53-7.34 (m, 7H), 7.17 (dd, J=1.6, 7.6 Hz, 1H), 6.96 (br s, 2H), 6.41 (dd, J=4.8, 7.6 Hz, 1H), 5.75 (s, 1H), 4.12-3.85 (m, 2H), 2.95-2.60 (m, 3H), 1.95 (br s, 1H), 1.76-1.67 (m, 2H), 1.48 (s, 9H).

Step 3. To a solution of tert-butyl 3-[4-[2-(2-amino-3-pyridyl)-5-phenyl-imidazo[4,5-b]pyridin-3-yl]phenyl]piperidine-1-carboxylate (500 mg, 0.915 mmol) in DCM (2.00 mL) was added TFA (2.0 mL, 1.83 mmol). The mixture was stirred at for 2 hrs. The mixture was concentrated to give 3-[5-phenyl-3-[4-(3-piperidyl)phenyl]imidazo[4,5-b]pyridin-2-yl]pyridin-2-amine (400 mg, 98% yield) as yellow oil. LCMS Rₜ=3.256 min in 4 min chromatography, Xtimate C18 2.1*30 mm, 3 μm, purity 98.7%, MS ESI calcd, for 446.22 [M+H]⁺ 447.22, found 447.1.

Step 4. To a solution of 3-[5-phenyl-3-[4-(3-piperidyl)phenyl]imidazo[4,5-b]pyridin-2-yl]pyridin-2-amine (200 mg, 0.448 mmol) in DMF (3 mL) was added methyl 4-(1-bromoethyl)benzoate (163 mg, 0.672 mmol) and DIEA (0.220 mL, 1.34 mmol). The mixture was stirred at 30° C. for 4 hrs. at which point water was added and the mixture was extracted with EtOAc (3×20 mL). The organic phase was washed with brine (3×10 mL), dried over Na₂SO₄ and concentrated. The residue was purified by column (EtOAc in PE, 0-99%) to give methyl 4-[1-[3-[4-[2-(2-amino-3-pyridyl)-5-phenyl-imidazo[4,5-b]pyridin-3-yl]phenyl]-1-piperidyl]ethyl]benzoate (150 mg, 55% yield) as a yellow solid. LCMS Rₜ=0.641 min in 4 min chromatography, Xtimate C18 2.1*30 mm, 3 μm, purity 100%, MS ESI calcd, for 608.29 [M+H]⁺ 609.29, found 609.2.

Step 5. To a solution of methyl 4-[1-[3-[4-[2-(2-amino-3-pyridyl)-5-phenyl-imidazo[4,5-b]pyridin-3-yl]phenyl]-1-piperidyl]ethyl]benzoate (150 mg, 0.246 mmol) in THE (1 mL) and water (1 mL) was added LiOH·H₂O (30.0 mg, 0.739 mmol). The mixture was stirred at 30° C. for 16 hrs. then concentrated in vacuo. The resulting residue was dissolved in DMSO (2 mL) and 1M HCl (2 mL) and purified by prep-HPLC (Welch Xtimate C18 150*30 mm*5 μm, water (FA)-ACN) to give 4-(1-(3-(4-(2-(2-aminopyridin-3-yl)-5-phenyl-3H-imidazo[4,5-b]pyridin-3-yl)phenyl)piperidin-1-yl)ethyl)benzoic acid 170 (49.0 mg, 33% yield) as a yellow solid. ¹H NMR (DMSO-d₆, 400 MHz) δ_{H}=8.25 (dd, J=4.0, 8.4 Hz, 1H), 8.04-7.94 (m, 4H), 7.91 (dd, J=2.4, 8.4 Hz, 2H), 7.53-7.32 (m, 9H), 7.18-7.08 (m, 1H), 6.94 (d, J=2.4 Hz, 2H), 6.44-6.31 (m, 1H), 3.76-3.57 (m, 1H), 3.10-2.76 (m, 3H), 2.17-1.81 (m, 3H), 1.80-1.52 (m, 2H), 1.51-1.40 (m, 1H), 1.35 (br d, J=6.0 Hz, 3H). HPLC Rₜ=3.820 min and 3.903 in 8 min chromatography, 0-60AB, purity 99.8%. LCMS Rₜ=2.901 and 2.938 min in 4 min chromatography, Xtimate C18 2.1*30 mm, 3 μm, purity 99.5%, MS ESI calcd. for 594.27 [M+H]⁺ 595.27, found 595.2.

### Example 139. Synthesis of 4-(1-(3-(4-(2-(2-aminopyridin-3-yl)-5-phenyl-3H-imidazo[4,5-b]pyridin-3-yl)phenyl) pyrrolidin-1-yl)ethyl)benzoic acid (Compound 171)

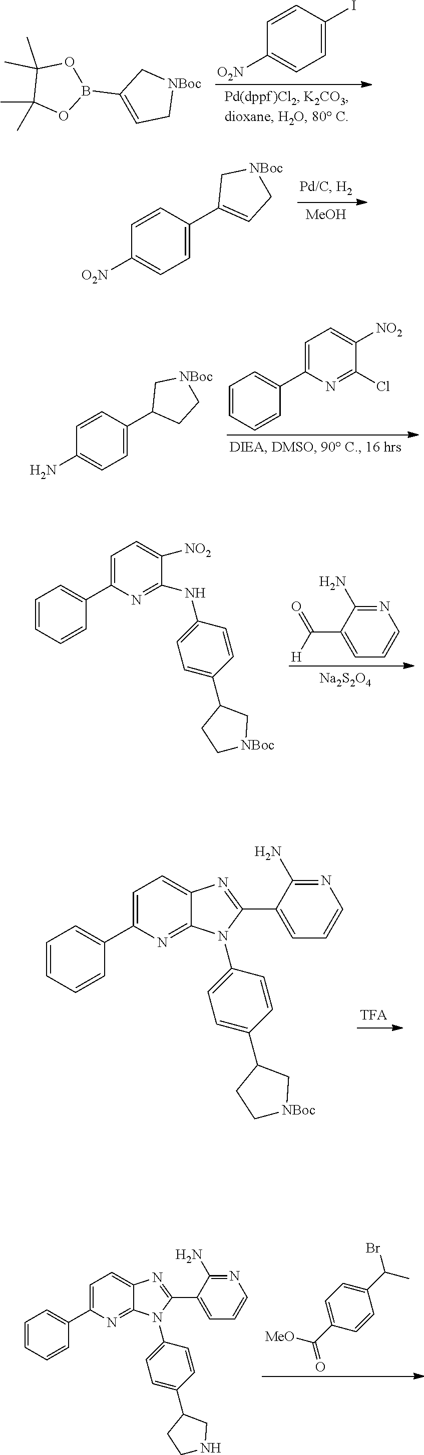

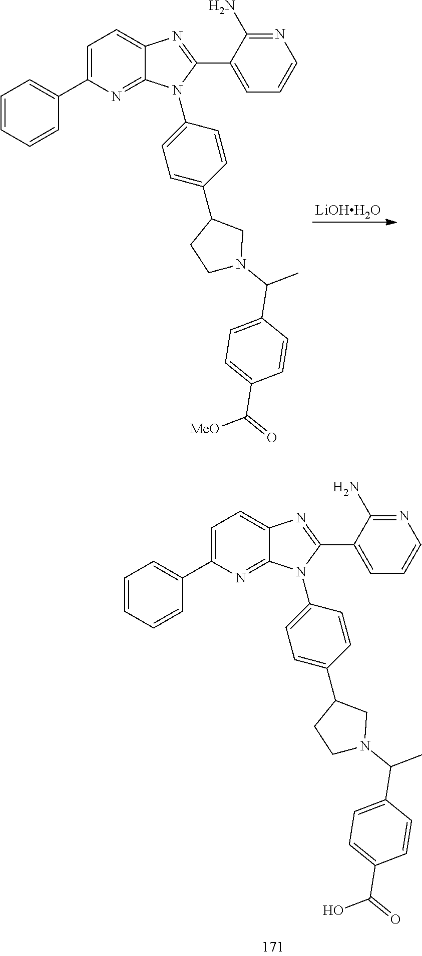 [SMILES: COC(=O)c1ccc(C(C)N2CCC(c3ccc(-n4c(-c5cccnc5N)nc5ccc(-c6ccccc6)nc54)cc3)C2)cc1 ; "LiOH·H2O" ; CC(c1ccc(C(=O)O)cc1)N1CCC(c2ccc(-n3c(-c4cccnc4N)nc4ccc(-c5ccccc5)nc43)cc2)C1]

Step 1. To a solution of tert-butyl 3-(4,4,5,5-tetramethyl-1,3,2-dioxaborolan-2-yl)-2,5-dihydropyrrole-1-carboxylate (5.00 g. 16.9 mmol) in 1,4-Dioxane (60 mL) and water (15 mL) was added 1-iodo-4-nitro-benzene (5.06 g, 20.3 mmol) Pd(dppf)Cl₂ (1.24 g, 1.69 mmol) and K₂CO₃ (5.85 g, 42.3 mmol) at 25° C. under N₂. The mixture was heated to 80° C. and stirred for 16 hrs. then filtered. H₂O (500 mL) was added to the filtrate and the product was extracted with EtOAc (3×500 mL). The organic phase was washed with brine (500 mL), dried over Na₂SO₄, filtered and concentrated in vacuo. The crude was purified by flash chromatography eluting with PE/EtOAc (from 0% to 20% of EtOAc in PE) to give tert-butyl 3-(4-nitrophenyl)-2,5-dihydropyrrole-1-carboxylate (2.50 g, 51% yield) as a yellow solid. ¹H NMR (400 MHz, DMSO-d₆) δ=8.22 (d, J=8.8 Hz, 2H), 7.74 (dd, J=4.8, 8.8 Hz, 2H), 6.71 (d, J=14.4 Hz, 1H), 4.50-4.43 (m, 2H), 4.26 (d, J=3.6 Hz, 2H), 1.45 (d, J=5.6 Hz, 9H).

Step 2. To the solution of tert-butyl 3-(4-nitrophenyl)-2,5-dihydropyrrole-1-carboxylate (1.00 g, 3.44 mmol) in ethyl acetate (40 mL) was added Pd/C (100 mg) at 25° C. The reaction was degassed and purged with Ar for 3 times, then degassed and purged with H₂ for 3 times and stirred under H₂ (50 Psi) for 16 hrs at 50° C. The reaction mixture was filtered and the cake was washed with MeOH (3×20 mL). The combined filtrate was concentrated in vacuo to give tert-butyl 3-(4-aminophenyl) pyrrolidine-1-carboxylate (700 mg, 78% yield) as a yellow solid.

Step 3. To a solution of 2-chloro-3-nitro-6-phenyl-pyridine (1.90 g. 8.10 mmol) in DMSO (30 mL) was added tert-butyl 3-(4-aminophenyl) pyrrolidine-1-carboxylate (2.55 g, 9.72 mmol) and DIEA (3.70 mL, 20.2 mmol) at 25° C. under N₂. The mixture was stirred for 16 hrs. at which point water (300 mL) was added and the mixture was extracted with EtOAc (3×300 mL). The organic phase was washed with brine (300 ml), dried over Na₂SO₄, filtered and concentrated in vacuo. The crude was purified by flash chromatography eluting with PE/EtOAc (from 0% to 30% of EtOAc in PE) to give tert-butyl 3-[4-[(3-nitro-6-phenyl-2-pyridyl)amino]phenyl]pyrrolidine-1-carboxylate (3.24 g, 87% yield) as red oil. ¹H NMR (400 MHz, DMSO-d₆) δ=10.10 (s, 1H), 8.60 (d, J=8.8 Hz, 1H), 8.09 (dd, J=2.0, 6.0 Hz, 2H), 7.71 (d, J=8.4 Hz, 2H), 7.58 (d, J=8.8 Hz, 1H), 7.55-7.52 (m, 3H), 7.34 (d, J=8.4 Hz, 2H), 3.72 (dd, J=7.6, 10.2 Hz, 1H), 3.53-3.44 (m, 1H), 3.43-3.36 (m, 1H), 3.21-3.15 (m, 1H), 2.28-2.16 (m, 1H), 1.98-1.90 (m, 1H), 1.42 (d, J=3.6 Hz, 9H).

Step 4. To solution of tert-butyl 3-[4-[(3-nitro-6-phenyl-2-pyridyl)amino]phenyl]pyrrolidine-1-carboxylate (3.24 g, 7.04 mmol) in DMSO (12 mL) and Methanol (3 mL) was added Na₂S₂O₄ (3.68 g, 21.1 mmol) and 2-aminopyridine-3-carbaldehyde (1.03 g, 8.45 mmol) at 25° C. under N₂. The mixture was heated to 100° C. and stirred for 16 hrs. EtOAc (100 mL) and MeOH (50 mL) were added and the mixture was filtered. The filtrate was concentrated in vacuo to give a crude. Water (100 mL) was added and the resulting mixture was extracted with EtOAc (3×100 mL). The organic phase was washed with brine (100 mL), dried over Na₂SO₄, filtered and concentrated in vacuo. The crude was purified by flash chromatography eluting with PE/EtOAc (from 0% to 40% of EtOAc in PE) to give 3-[5-phenyl-3-(4-pyrrolidin-3-ylphenyl) imidazo[4,5-b]pyridin-2-yl]pyridin-2-amine (246 mg, 8% yield) as a yellow solid. LCMS Rₜ=0.944 min in 1.5 min chromatography, purity 94%, MS ESI calcd, for 532.26 [M−1Bu+H]⁺ 477.26, found 477.1.

Step 5. To a solution of tert-butyl 3-[4-[2-(2-amino-3-pyridyl)-5-phenyl-imidazo[4,5-b]pyridin-3-yl]phenyl]pyrrolidine-1-carboxylate (720 mg, 1.35 mmol) in DCM (5 mL) was added TFA (5 mL, 1.35 mmol) at 25° C. The mixture was stirred at 25° C. for 16 hrs. then concentrated in vacuo to give 3-[5-phenyl-3-(4-pyrrolidin-3-ylphenyl) imidazo[4,5-b]pyridin-2-yl]pyridin-2-amine (1.30 g, crude, TFA salt) as green oil, which was directly used in the next step without purification. ¹H NMR (400 MHz, DMSO-d₆) δ=8.35 (d, J=8.4 Hz, 1H), 8.12 (dd, J=1.2, 6.0 Hz, 1H), 8.08-8.02 (m, 3H), 7.87 (dd, J=1.6, 7.6 Hz, 1H), 7.61-7.54 (m, 4H), 7.51-7.46 (m, 2H), 7.45-7.39 (m, 1H), 6.88 (dd, J=6.4, 7.6 Hz, 1H), 6.06-6.01 (m, 2H), 3.77-3.67 (m, 1H), 3.62-3.53 (m, 1H), 3.51-3.41 (m, 1H), 3.34-3.24 (m, 1H), 3.22-3.12 (m, 1H), 2.48-2.40 (m, 1H), 2.06-1.97 (m, 1H).

Step 6. To a solution of 3-[5-phenyl-3-(4-pyrrolidin-3-ylphenyl) imidazo[4,5-b]pyridin-2-yl]pyridin-2-amine (200 mg, 0.462 mmol) in DMF (3 mL) was added TEA (2 mL). Then methyl 4-(1-bromoethyl)benzoate (135 mg, 0.555 mmol) and DIEA (0.26 mL, 1.39 mmol) was added at 25° C. under N₂. The mixture was stirred at 25° C. for 16 hrs, at which point water (20.0 mL) was added. The mixture was extracted with EtOAc (3×20 mL) and the combined organic phase was washed with brine (20 mL), dried over Na₂SO₄, filtered and concentrated in vacuo. The crude was purified by flash chromatography eluting with PE/EIOAc (from 0% to 100% of EtOAc in PE) to give methyl 4-[1-[3-[4-[2-(2-amino-3-pyridyl)-5-phenyl-imidazo[4,5-b]pyridin-3-yl]phenyl]pyrrolidin-1-yl]ethyl]benzoate (110 mg, 40% yield) as yellow oil. ¹H NMR (400 MHz, DMSO-d₆) δ=8.26 (dd, J=2.4, 8.4 Hz, 1H), 8.04-7.90 (m, 6H), 7.51 (dd, J=2.4, 8.4 Hz, 2H), 7.49-7.43 (m, 4H), 7.42-7.36 (m, 3H), 7.17 (td, J=2.0, 7.6 Hz, 1H), 6.95 (d, J=3.2 Hz, 2H), 6.39 (ddd, J=2.4, 4.8, 7.6 Hz, 1H), 4.03 (q, J=7.2 Hz, 1H), 3.83 (d, J=2.0 Hz, 3H), 2.83-2.72 (m, 2H), 2.64-2.52 (m, 2H), 2.46-2.23 (m, 2H), 1.89-1.76 (m, 1H), 1.34 (dd, J=4.0, 6.4 Hz, 3H).

Step 7. To a solution of methyl 4-[1-[3-[4-[2-(2-amino-3-pyridyl)-5-phenyl-imidazo[4,5-b]pyridin-3-yl]phenyl]pyrrolidin-1-yl]ethyl]benzoate (110 mg, 0.185 mmol) in THF (1.5 mL) and Water (1.5 mL) was added LiOH. H₂O (19.0 mg, 0.462 mmol) at 25° C. The mixture was stirred at 25° C. for 16 hrs. at which point it was concentrated in vacuo. The crude material was purified by prep-HPLC (Welch Xtimate C18 150*30 mm*5 μm water (FA)-ACN) to give 4-[1-[3-[4-[2-(2-amino-3-pyridyl)-5-phenyl-imidazo[4,5-b]pyridin-3-yl]phenyl]pyrrolidin-1-yl]ethyl]benzoic acid 171 (79.0 mg, 74% yield) as a yellow solid. ¹H NMR (400 MHz, DMSO-d₆) δ=8.23 (dd, J=2.0, 8.4 Hz, 1H), 8.17 (s, 1H), 8.01-7.93 (m, 4H), 7.89 (dd, J=4.0, 8.4 Hz, 2H), 7.50-7.41 (m, 6H), 7.40-7.35 (m, 3H), 7.17 (td, J=1.6, 7.2 Hz, 1H), 6.39 (ddd, J=3.2, 4.8, 7.6 Hz, 1H), 3.53-3.47 (m, 1H), 3.45-3.36 (m, 1H), 2.94-2.79 (m, 2H), 2.69-2.57 (m, 1H), 2.57-2.53 (m, 1H), 2.34-2.25 (m, 1H), 1.87-1.76 (m, 1H), 1.35 (d, J=6.4 Hz, 3H). HPLC Rₜ=2.549 min in 8.0 min chromatography, 10-80AB_8 min.1 cm, purity 47.1%. R (=2.577 min in 8.0 min chromatography, 10-80AB_8 min.1 cm, purity 53%. LCMS Rₜ=2.238 min in 4.0 min chromatography, purity 100%, MS ESI calcd, for 580.26 [M+H]⁺ 581.26, found 581.1.

### Example 140. Synthesis of 4-((5-(2-(2-aminopyridin-3-yl)-5-(4-fluorophenyl)-3H-imidazo[4,5-b]pyridin-3-yl)pyridin-2-yl)carbamoyl)benzoic acid (Compound 109)

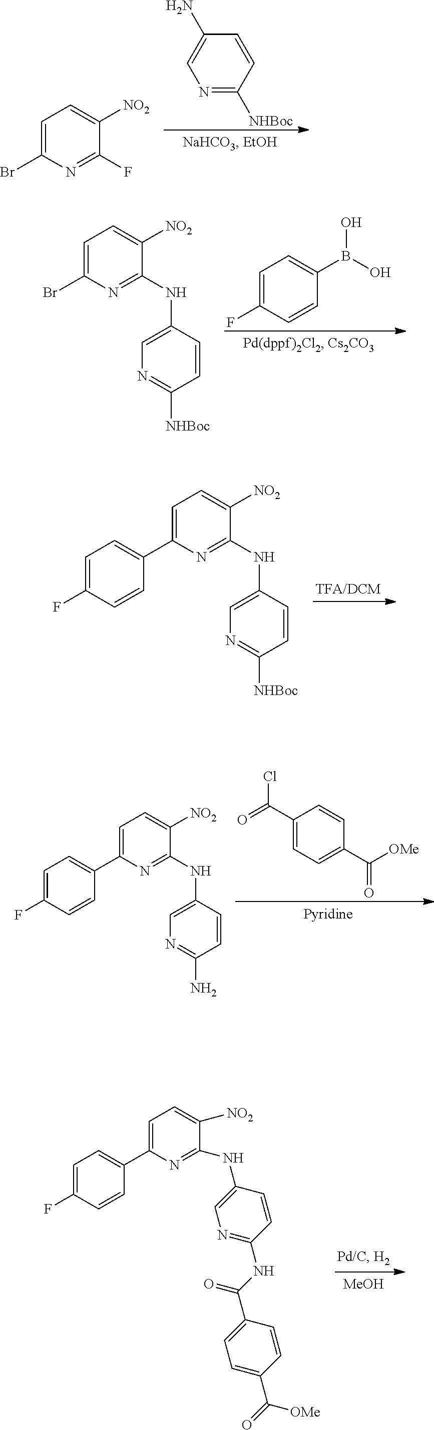

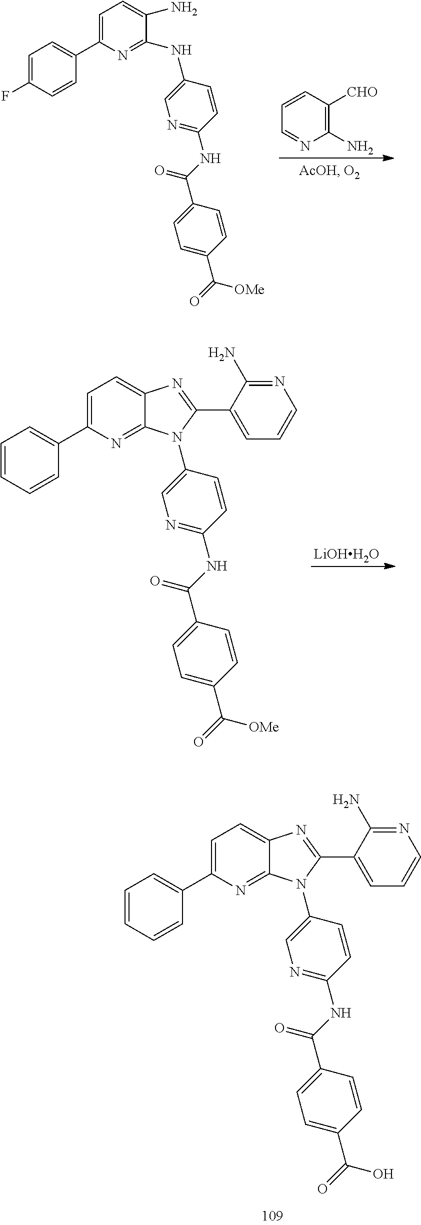 [SMILES: COC(=O)c1ccc(C(=O)Nc2ccc(Nc3nc(-c4ccc(F)cc4)ccc3N)cn2)cc1 ; Nc1ncccc1C=O ; COC(=O)c1ccc(C(=O)Nc2ccc(-n3c(-c4cccnc4N)nc4ccc(-c5ccccc5)nc43)cn2)cc1 ; "LiOH·H2O" ; Nc1ncccc1-c1nc2ccc(-c3ccccc3)nc2n1-c1ccc(NC(=O)c2ccc(C(=O)O)cc2)nc1]

Step 1. To a solution of 6-bromo-2-fluoro-3-nitropyridine (1.00 g, 4.53 mmol) in ethanol (10 mL) was added NaHCO₃ (0.460 g, 4.53 mmol) and tert-butyl N-(5-amino-2-pyridyl)carbamate (0.850 g, 4.07 mmol) at 25° C. The mixture was stirred at 25° C. for 16 hrs.

at which point water (5 mL) was added to the reaction mixture. The mixture was pulping with isopropyl ether (10 mL) for 10 mins. The reaction mixture was filtered and The filter cake was concentrated to give tert-butyl (5-((6-bromo-3-nitropyridin-2-yl)amino)pyridin-2-yl)carbamate (1.80 g, 96.9% yield) as a yellow solid. ¹H NMR (DMSO-d₆, 400 MHz) δ_{H}=10.09 (s, 1H), 9.79 (s, 1H), 8.43-8.36 (m, 2H), 7.89-7.78 (m, 2H), 7.12 (d, J=8.4 Hz, 1H), 1.48 (s, 9H).

Step 2. To a solution of tert-butyl (5-((6-bromo-3-nitropyridin-2-yl)amino)pyridin-2-yl)carbamate (1.80 g, 4.39 mmol) and (4-fluorophenyl) boronic acid (0.800 g. 5.70 mmol) in 1,4-Dioxane (30 mL) and water (6 mL) was added Cs₂CO₃ (4.29 g, 13.2 mmol) and Pd(dppf)Cl₂ (0.32 g, 0.439 mmol). The mixture was heated to 100° C. and stirred for 12 hrs. then concentrated and water (S mL) was added to the resulting residue. The mixture was extracted with ErOAc (3×10 mL) and the combined organic phase was washed with brine (10 mL), dried over anhydrous Na₂SO₄, filtered and concentrated. The residue was purified by flash silica gel chromatography (eluent of 0~15% EtOAc in PE) to give tert-butyl (5-((6-(4-fluorophenyl)-3-nitropyridin-2-yl)amino)pyridin-2-yl)carbamate (800 mg, 43% yield) as a yellow solid. ¹H NMR (DMSO-d₆, 400 MHz) δ_{H}=10.06 (s, 1H), 9.80 (s, 1H), 8.64-8.50 (m, 2H), 8.14-8.02 (m, 3H), 7.86 (d, J=9.2 Hz, 1H), 7.57 (d, J=8.8 Hz, 1H), 7.36 (m, 2H), 1.49 (s, 9H).

Step 3. To a solution of tert-butyl (5-((6-(4-fluorophenyl)-3-nitropyridin-2-yl)amino)pyridin-2-yl)carbamate (2.00 g, 4.70 mmol) in DCM (20 mL) was added TFA (2.0 mL, 4.70 mmol). The mixture was stirred at 25° C. for 12 hrs. then concentrated directly to give N⁵-(6-(4-fluorophenyl)-3-nitropyridin-2-yl)pyridine-2,5-diamine (1.50 g, 92% yield) as yellow oil, which was used in the next step directly. LCMS Rₜ=0.412 min in 1 min chromatography, purity 71.0%, MS ESI calcd, for 325.30, [M+H]⁺ 326.31, found 326.10.

Step 4. To a solution of N₅-(6-(4-fluorophenyl)-3-nitropyridin-2-yl)pyridine-2,5-diamine (280 mg, 0.861 mmol) in Pyridine (10 mL) was added methyl 4-chlorocarbonylbenzoate (205 mg, 1.03 mmol). The mixture was heated to 80° C. and stirred for 12 hrs. Water (5 mL) was added to the residue and the resulting mixture was extracted with EtOAc (3×10 mL). The combined organic phase was washed with brine (10 mL), dried over anhydrous Na₂SO₄, filtered and concentrated. The residue was purified by flash silica gel chromatography (eluent of 0~12% MeOH in DCM) to give methyl 4-((5-((6-(4-fluorophenyl)-3-nitropyridin-2-yl)amino)pyridin-2-yl)carbamoyl)benzoate (420 mg, 75% yield) as light yellow oil. ¹H NMR (DMSO-d₆, 400 MHz) δ_{H}=11.10 (s, 1H), 10.16 (s, 1H), 8.79-8.74 (m, 1H), 8.63 (d, J=8.8 Hz, 1H), 8.31-8.25 (m, 1H), 8.21-8.06 (m, 7H), 7.62 (d, J=8.8 Hz, 1H), 7.37 (t, J=8.8 Hz, 2H), 3.90 (s, 3H).

Step 5. To a solution of methyl 4-((5-((6-(4-fluorophenyl)-3-nitropyridin-2-yl)amino)pyridin-2-yl)carbamoyl)benzoate (400 mg, 0.821 mmol) in THF (10 mL) was added Pd/C (100 mg, purity 10%) under N₂. The reaction mixture was degassed and purged with H₂ (15 psi) for 3 times. The mixture was stirred at 25° C. for 12 hrs under H₂ (15 psi). The reaction mixture was filtered and the filtrate was concentrated directly to give methyl 4-((5-((3-amino-6-(4-fluorophenyl)pyridin-2-yl)amino)pyridin-2-yl)carbamoyl)benzoate (220 mg, 58% yield) as light yellow oil, which was used in the next step directly. ¹H NMR (DMSO-d₆, 400 MHz) ôH=10.89 (s, 1H), 8.93 (s, 1H), 8.17-8.04 (m, 7H), 7.98-7.90 (m, 2H), 7.29-7.18 (m. 3H), 7.01 (d, J=7.6 Hz, 1H), 5.29 (s, 2H), 3.90 (s, 3H).

Step 6. To a solution of methyl 4-((5-((3-amino-6-(4-fluorophenyl)pyridin-2-yl) amino)pyridin-2-yl)carbamoyl)benzoate (220 mg, 0.481 mmol) in acetic acid (3 mL) was added 2-aminopyridine-3-carbaldehyde (79.0 mg, 0.649 mmol) under 02. The mixture was stirred at 80° C. for 1 hr under 02. The reaction mixture was concentrated directly. The residue was purified by flash silica gel chromatography (eluent of 0~5% MeOH in DCM) to give methyl 4-((5-(2-(2-aminopyridin-3-yl)-5-(4-fluorophenyl)-3H-imidazo[4,5-b]pyridin-3-yl) pyridin-2-yl)carbamoyl)benzoate (260 mg, 97% yield) as light yellow oil. ¹H NMR (DMSO-do, 400 MHz) δ_{H}=11.26 (s, 1H), 8.52 (d, J=2.4 Hz, 1H), 8.40-8.27 (m, 2H), 8.17-8.14 (m, 2H), 8.12-8.07 (m, 4H), 8.06-7.99 (m, 3H), 7.40 (dd, J=1.6, 7.6 Hz, 1H), 7.31 (t, J=8.8 Hz. 2H), 6.86-6.71 (m, 2H), 6.53 (dd, J=4.8, 7.6 Hz, 1H). 3.90 (s, 3H).

Step 7. To a solution of methyl 4-((5-(2-(2-aminopyridin-3-yl)-5-(4-fluorophenyl)-3H-imidazo[4,5-b]pyridin-3-yl)pyridin-2-yl)carbamoyl)benzoate (260 mg, 0.465 mmol) in THF (3 mL) and water (3 mL) was added LiOH H₂O (59.0 mg, 1.39 mmol). The mixture was stirred at 25° C. for 2 hrs. then concentrated directly. The residue was purified by prep-HPLC (Column: Welch Xtimate C18 150*30 mm*5 μm; mobile phase: [water (FA)-ACN]; B %: 0%-35%; 25 min) to give 4-((5-(2-(2-aminopyridin-3-yl)-5-(4-fluorophenyl)-3H-imidazo[4,5-b]pyridin-3-yl)pyridin-2-yl)carbamoyl)benzoic acid 109 (10.9 mg, 4% yield) as a light yellow solid. ¹H NMR (DMSO-d₆, 400 MHz) δ_{H}=11.23 (s, 1H), 8.52 (d, J=2.4 Hz, 1H). 8.39-8.27 (m, 2H), 8.15-8.00 (m, 9H), 7.43-7.27 (m, 3H), 6.82 (s, 2H), 6.53 (dd, J=4.8, 7.6 Hz, 1H). HPLC Rₜ=2.734 min in 8 min chromatography, purity 100%. LCMS Rₜ=1.240 min in 2 min chromatography, purity 98.5%, MS ESI calcd, for 545.16, [M+H]⁺ 546.16, found 546.1.

### Example 141. Synthesis of 4-((5-(2-(2-aminopyridin-3-yl)-6-isopropyl-1H-benzo[d]imidazol-1-yl)pyridin-2-yl)carbamoyl)benzoic acid (Compound 172)

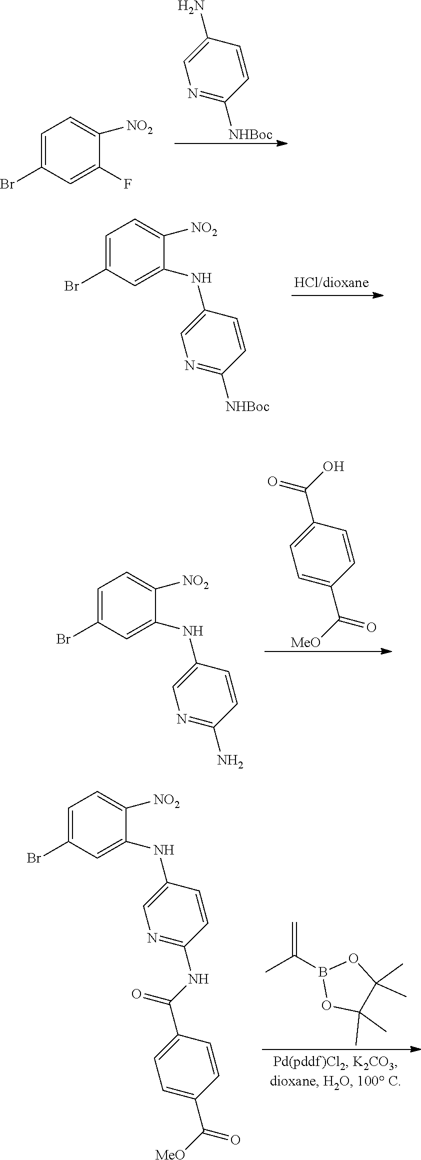

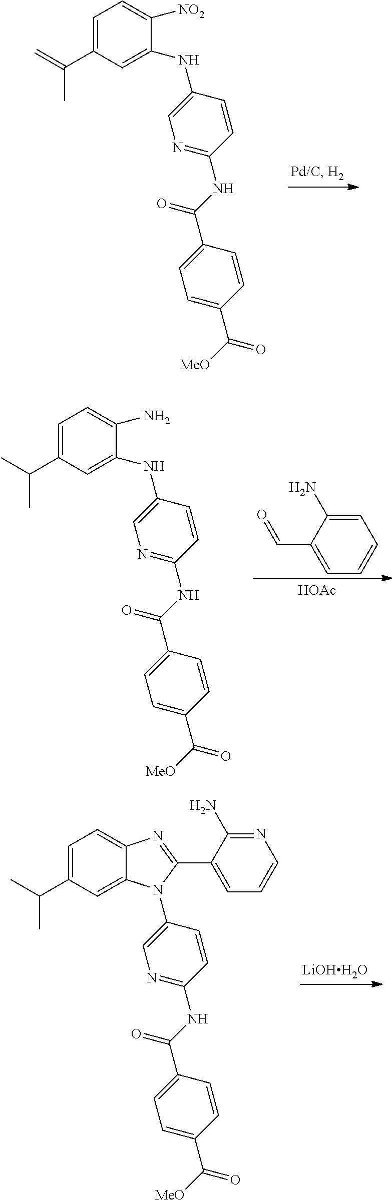 [SMILES: C=C(C)c1ccc([N+](=O)[O-])c(Nc2ccc(NC(=O)c3ccc(C(=O)OC)cc3)nc2)c1 ; COC(=O)c1ccc(C(=O)Nc2ccc(Nc3cc(C(C)C)ccc3N)cn2)cc1 ; Nc1ccccc1C=O ; COC(=O)c1ccc(C(=O)Nc2ccc(-n3c(-c4cccnc4N)nc4ccc(C(C)C)cc43)cn2)cc1 ; "LiOH·H2O" ; "HOAc"]

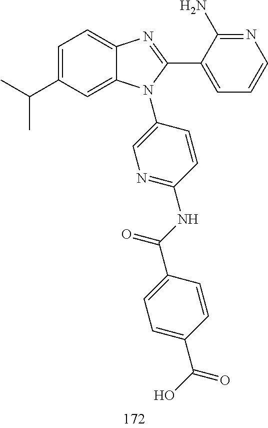 [SMILES: CC(C)c1ccc2nc(-c3cccnc3N)n(-c3ccc(NC(=O)c4ccc(C(=O)O)cc4)nc3)c2c1]

Step 1. To a solution of 4-bromo-2-fluoro-1-nitrobenzene (3.00 g, 13.6 mmol) in1, 4-Dioxane (30 mL) was added tert-butyl (5-aminopyridin-2-yl)carbamate (2.85 g, 13.6 mmol) and NaHCO₃ (1.15 g, 13.6 mmol) at 25° C. The mixture was heated to 80° C. and stirred for 14 hrs. then filtered. The filter cake was dried and then stirred in ACN (10 mL) for 10 mins. The reaction mixture was filtered and the filter cake was dried affording tert-butyl (5-((5-bromo-2-nitrophenyl)amino)pyridin-2-yl)carbamate (1.19 g, 21%) as a yellow solid. ¹H NMR (400 MHz, DMSO-d₆) δ=9.89 (s, 1H), 9.47 (s, 1H), 8.21 (d, J=2.0 Hz, 1H), 8.05 (d, J=9.6 Hz, 1H), 7.87 (d, J=8.8 Hz, 1H), 7.72 (dd, J=2.4, 8.8 Hz, 1H), 7.01-6.94 (m, 2H), 1.49 (s, 9H).

Step 2. To a solution of tert-butyl (5-((5-bromo-2-nitrophenyl)amino)pyridin-2-yl)carbamate (1.19 g, 2.91 mmol) in DCM (2 mL) was added HCl/dioxane (20 mL, 2 M). The mixture was stirred at 25° C. for 14 hrs. The reaction mixture was concentrated directly to afford N₅-(5-bromo-2-nitrophenyl)pyridine-2,5-diamine (920 mg, crude) as a yellow solid, which was used to the next step without purification. ¹H NMR (400 MHz, DMSO-d₆) δ=9.34 (3, 1H), 8.19-8.10 (m, 1H), 8.04 (d, J=9.2 Hz, 1H), 7.99 (s, 1H), 7.82 (d, J=8.8 Hz, 1H), 7.14-6.97 (m, 4H).

Step 3. To a mixture of N₅-(5-bromo-2-nitrophenyl)pyridine-2,5-diamine (920 mg, 2.98 mmol) in Pyridine (10 mL) was added 4-(methoxycarbonyl)benzoic acid (751 mg, 4.17 mmol), EDCI (1.26 g. 8.93 mmol). The mixture was heated to 80° C. and stirred for 2 hrs. Water (10 mL) was added into the mixture and the resulting mixture was filtered and the filter cake was dried. The crude product was stirred with ACN (5 mL) for 14 hrs and filtered. The filter cake was dried to afford methyl 4-((5-((5-bromo-2-nitrophenyl)amino)pyridin-2-yl)carbamoyl)benzoate (480 mg, 34%) as a yellow solid. ¹H NMR (400 MHz, DMSO-d₆) δ=11.14 (s, 1H), 9.54 (s, 1H), 8.41 (d, J=1.6 Hz, 1H), 8.27 (d, J=8.8 Hz, 1H), 8.18-8.04 (m, 5H), 7.92-7.86 (m, 1H), 7.14-7.09 (m, 1H), 7.06 (dd, J=1.6, 8.8 Hz, 1H), 3.91 (s, 3H).

Step 4. To a solution of methyl 4-((5-((5-bromo-2-nitrophenyl)amino)pyridin-2-yl)carbamoyl)benzoate (480 mg, 1.02 mmol) in1,4-Dioxane (10 mL) and water (2 mL) was added Pd(dppf)Cl₂ (37.0 mg, 0.0500 mmol) and 4,4,5,5-tetramethyl-2-(prop-1-en-2-yl)-1,3,2-dioxaborolane (205 mg, 1.22 mmol) at 25° C. The mixture was heated to 100° C. and stirred for 14 hrs under N₂ atmosphere. The reaction mixture was concentrated directly. The residue was purified by combi-flash column (EtOAc in PE=0%~100%) to afford methyl 4-((5-((2-nitro-5~ (prop-1-en-2-yl)phenyl)amino)pyridin-2-yl)carbamoyl)benzoate (160 mg, 36% yield) as yellow oil. ¹H NMR (400 MHz, DMSO-d₆) δ=11.14 (s, 1H), 9.55 (s, 1H), 8.48 (d, J=2.4 Hz, 1H), 8.30 (d, J=8.8 Hz, 1H), 8.20-8.10 (m, 5H), 7.94 (dd, J=2.8, 9.2 Hz, 1H), 7.19-7.08 (m, 2H), 5.55 (s, 1H), 5.31 (s, 1H), 3.94 (s, 3H), 2.04 (s, 3H).

Step 5. To a solution of methyl 4-((5-((2-nitro-5-(prop-1-en-2-yl)phenyl)amino)pyridin-2-yl)carbamoyl)benzoate (160 mg, 0.370 mmol) in methanol (5 mL) was added wet Pd/C (10%, 0.1 g) under N₂ atmosphere. The suspension was degassed and purged with H₂ for 3 times then stirred under H₂ (50 psi) at 80° C. for 12 hrs. The reaction mixture was filtered with THE (20 mL) and the filtrate was concentrated to afford methyl 4-((5-((2-amino-5-isopropylphenyl)amino)pyridin-2-yl)carbamoyl)benzoate (150 mg, crude) as a brown solid. ¹H NMR (400 MHz, DMSO-d₆) δ=10.68 (s, 1H), 8.19-8.13 (m, 2H), 8.12-8.03 (m, 3H), 7.98 (d, J=8.8 Hz, 1H), 7.93 (d, J=2.8 Hz, 1H), 7.34 (s, 1H), 7.21-7.16 (m, 1H), 6.94 (d, J=1.6 Hz, 1H), 6.82-6.78 (m, 1H), 6.77-6.73 (m, 1H), 4.64 (s, 2H), 3.95 (s, 3H), 1.22-1.17 (m, 6H).

Step 6. To a solution of methyl 4-((5-((2-amino-5-isopropylphenyl)amino)pyridin-2-yl)carbamoyl)benzoate (160 mg, 0.39 mmol) in acetic acid (2 mL) was added 2-aminobenzaldehyde (72.0 mg, 0.590 mmol). The reaction mixture was degassed and purged with O₂ for 3 times. The mixture was heated to 80° C. and stirred for 2 hrs. then concentrated directly to afford methyl 4-((5-(2-(2-aminopyridin-3-yl)-6-isopropyl-1H-benzo[d]imidazol-1-yl)pyridin-2-yl)carbamoyl)benzoate (150 mg, 60% yield) as a brown oil. LCMS Rₜ=0.894 min in 1.5 min chromatography, purity 52.65%, MS ESI calcd, for 506.21 [M+H]⁺ 507.21, found 507.2.

Step 7. To a solution of methyl 4-((5-(2-(2-aminopyridin-3-yl)-6-isopropyl-1H-benzo[d]imidazol-1-yl)pyridin-2-yl)carbamoyl)benzoate (150 mg, 0.290 mmol) in THF (2 mL), water (2 mL) was added LiOH·H₂O (124 mg, 2.96 mmol) at 25° C. The mixture was stirred at 25° C. for 2 hrs. HCOOH (2 mL) was added to the mixture. The resulting suspension was extracted with DCM (3×35 mL). The combined organic phase was dried over anhydrous Na₂SO₄, filtered and concentrated. The residue was purified by prep-HPLC (condition: water (FA)-ACN; Column: Xtimate C18 40*200 mm*7 μm; B %: 6%-46%; Gradient Time, 25 min) to afford 4~ ((5-(2-(2-aminopyridin-3-yl)-6-isopropyl-1H-benzo[d]imidazol-1-yl)pyridin-2-yl)carbamoyl)benzoic acid 172 (37.0 mg, 34%) as a yellow solid. ¹H NMR (400 MHz, DMSO-d₆) δ=11.25 (s, 1H), 8.41 (d, J=2.4 Hz, 1H), 8.38 (d, J=8.8 Hz, 1H), 8.14-8.09 (m, 2H), 8.08-8.02 (m, 3H), 7.98 (dd, J=1.6, 4.8 Hz, 1H), 7.73 (d, J=8.0 Hz, 1H), 7.29-7.22 (m, 2H), 7.09 (s, 1H), 6.83 (s, 2H), 6.46 (dd, J=4.8, 7.6 Hz, 1H), 3.06-2.98 (m, 1H), 1.23 (d, J=6.8 Hz, 6H). HPLC Rₜ=2.624 min in 8 min chromatography, purity 95.9%. LCMS Rₜ=1.749 min in 4 min chromatography, purity 99.1%, MS ESI calcd, for 492.19 [M+H]⁺ 493.19, found 493.2.

### Example 142. Synthesis of 4-((3-(4-(2-(2-aminopyridin-3-yl)-5-isopropyl-3H-imidazo[4,5-b]pyridin-3-yl)phenyl) azetidin-1-yl)methyl)benzoic acid (Compound 173)

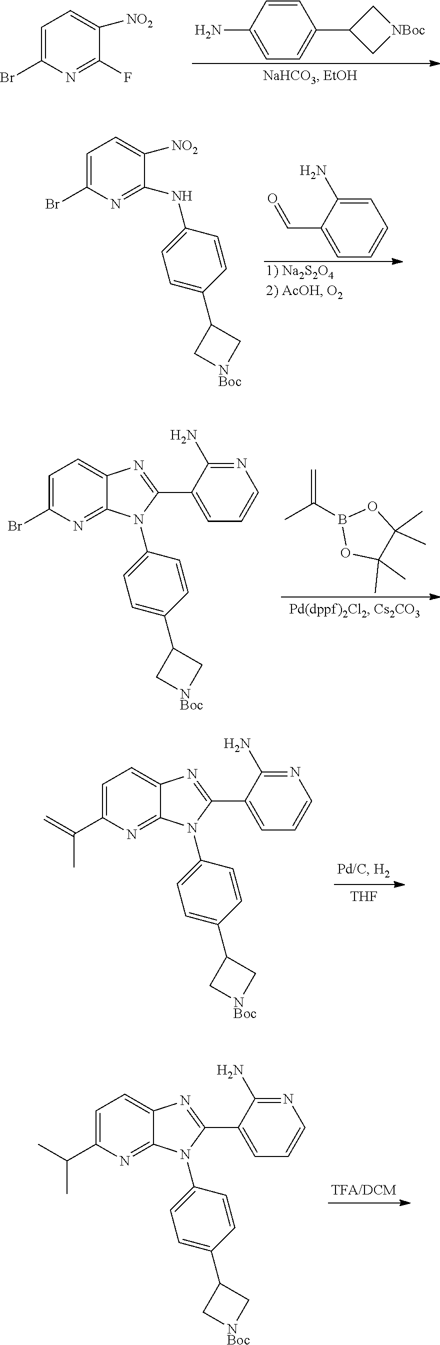

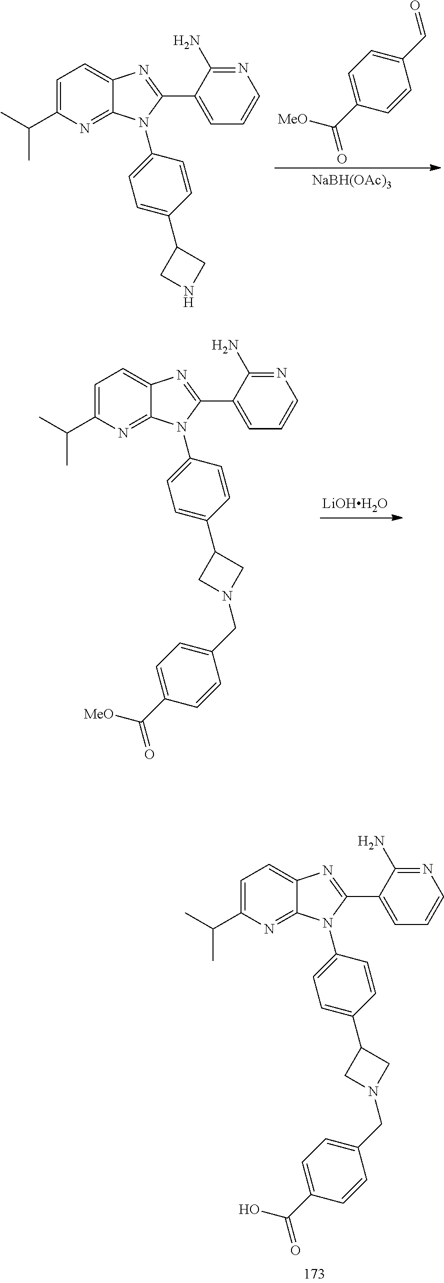 [SMILES: CC(C)c1ccc2nc(-c3cccnc3N)n(-c3ccc(C4CNC4)cc3)c2n1 ; COC(=O)c1ccc(C=O)cc1 ; COC(=O)c1ccc(CN2CC(c3ccc(-n4c(-c5cccnc5N)nc5ccc(C(C)C)nc54)cc3)C2)cc1 ; "LiOH·H2O" ; "NaBH(OAc)3" ; CC(C)c1ccc2nc(-c3cccnc3N)n(-c3ccc(C4CN(Cc5ccc(C(=O)O)cc5)C4)cc3)c2n1]

Step 1. To a solution of 6-bromo-2-fluoro-3-nitropyridine (1.00 g, 4.53 mmol) in ethanol (10 mL) was added NaHCO₃ (458 mg, 4.53 mmol) and tert-butyl 3-(4-aminophenyl) azetidine-1-carboxylate (1.01 g, 4.07 mmol) at 25° C. The mixture was stirred at 25° C. for 16 hrs. then diluted with water (5 mL). The mixture was stirred in isopropyl ether (50 mL) for 10 mins then filtered. The filter cake was dried giving tert-butyl 3-(4-((6-bromo-3-nitropyridin-2-yl)amino)phenyl) azetidine-1-carboxylate (2.00 g, 98% yield) as a yellow solid. ¹H NMR (DMSO-d₆, 400 MHz) δ_{H}=10.09 (s, 1H), 8.40 (d, J=8.4 Hz, 1H), 7.58 (d, J=8.4 Hz, 2H), 7.37 (d, J=8.4 Hz, 2H), 7.14 (d, J=8.4 Hz, 1H), 4.25 (s, 2H), 3.90-3.76 (m, 3H), 1.41 (s, 9H).

Step 2. To a solution of tert-butyl 3-(4-((6-bromo-3-nitropyridin-2-yl)amino)phenyl) azetidine-1-carboxylate (1.50 g. 3.34 mmol) and 2-aminopyridine-3-carbaldehyde (0.550 g, 4.51 mmol) in DMSO (10 mL) and Methanol (10 mL) was added Na₂S₂O₄ (1.74 g, 10.0 mmol). The mixture was heated to 100° C. and stirred for 12 hrs. The reaction mixture was concentrated. Aq. LiCl (10 mL) was added to the residue and the product was extracted with EtOAc (3×10 mL). The combined organic phase was washed with brine (20 mL), dried over anhydrous Na₂SO₄, filtered and concentrated. Acetic acid (3 mL) and 2-aminopyridine-3-carbaldehyde (0.550 g, 4.51 mmol) was added into the residue. The mixture was stirred at 80° C. for 1 hr under 02. The residue was purified by flash silica gel chromatography (eluent of 0~5% MeOH in DCM) to give tert-butyl 3-(4-(2-(2-aminopyridin-3-yl)-5-bromo-3H-imidazo[4,5-b]pyridin-3-yl)phenyl) azetidine-1-carboxylate (110 mg, 6% yield) as a yellow solid. ¹H NMR (DMSO-d₆, 400 MHz) δ_{H}=8.17 (d, J=8.4 Hz, 1H), 7.99 (d, J=3.6 Hz, 1H), 7.57 (d, J=8.4 Hz, 1H), 7.52-7.49 (m, 2H), 7.42 (d, J=8.4 Hz, 2H), 7.21 (d, J=6.8 Hz, 1H), 6.86 (s, 2H), 6.42 (dd, J=4.8, 7.6 Hz, 1H), 4.28 (s, 2H), 3.90 (s, 3H), 1.40 (s, 9H).

Step 3. To a solution of tert-butyl 3-(4-(2-(2-aminopyridin-3-yl)-5-bromo-3H-imidazo[4,5-b]pyridin-3-yl)phenyl) azetidine-1-carboxylate (200 mg, 0.384 mmol) and 2-isopropenyl-4,4,5,5-tetramethyl-1,3,2-dioxaborolane (84.0 mg, 0.499 mmol) in 1,4-Dioxane (15.0 mL) and water (2 mL) was added Cs₂CO₃ (375 mg, 1.15 mmol) and Pd(dppf)Cl₂ (28 mg, 0.0384 mmol). The mixture was heated to 90° C. and stirred for 12 hrs. The reaction mixture was then concentrated and water (5 mL) was added to the residue. The resulting mixture was extracted with EtOAc (3×10 mL). The combined organic phase was washed with brine (10 mL), dried over anhydrous Na₂SO₄, filtered and concentrated. The residue was purified by flash silica gel chromatography (eluent of 0~70% EtOAc in PE) to give tert-butyl 3-(4-(2-(2-aminopyridin-3-yl)-5-(prop-1-en-2-yl)-3H-imidazo[4,5-b]pyridin-3-yl)phenyl) azetidine-1-carboxylate (120 mg, 65% yield) as a yellow solid. ¹H NMR (DMSO-d₆, 400 MHz) δ_{H}=8.15 (d, J=8.4 Hz, 1H), 7.99 (dd, J=1.6, 4.8 Hz, 1H), 7.66 (d, J=8.4 Hz, 1H), 7.51-7.39 (m, 4H), 7.18 (dd, J=1.6, 7.6 Hz, 1H), 6.95-6.90 (m, J=4.4 Hz, 1H), 6.42 (dd, J=4.8, 7.6 Hz, 1H), 5.84 (s, 1H), 5.26 (s, 1H), 4.28 (s, 2H), 3.95-3.84 (m, 3H), 2.13 (s, 3H), 1.40 (s, 9H).

Step 4. To a solution of tert-butyl 3-(4-(2-(2-aminopyridin-3-yl)-5-(prop-1-en-2-yl)-3H-imidazo[4,5-b]pyridin-3-yl)phenyl) azetidine-1-carboxylate (120 mg, 0.249 mmol) in THE (10 mL) was added wet Pd/C (50 mg, purity 10%) under N₂. The reaction mixture was degassed and purged with H₂ (15 psi) for 3 times. The mixture was stirred at 25° C. for 12 hrs under H₂ (15 psi). The reaction mixture was filtered and the filtrate was concentrated directly to give tert-butyl 3-(4-(2-(2-aminopyridin-3-yl)-5-isopropyl-3H-imidazo[4,5-b]pyridin-3-yl)phenyl) azetidine-1-carboxylate (100 mg, 83% yield) as a yellow solid, which was used in the next step directly. ¹H NMR (DMSO-d₆, 400 MHz) δ_{H}=8.14-7.91 (m, 2H), 7.51-7.37 (m, 4H), 7.30 (d, J=8.4 Hz, 1H), 7.14 (dd, J=1.6, 7.6 Hz, 1H), 6.86 (s, 2H), 6.40 (dd, J=4.8, 7.6 Hz, 1H), 4.28 (s, 2H), 3.89 (s, 3H), 3.06 (td, J=6.8, 13.6 Hz, 1H), 1.41 (s, 9H), 1.23 (d, J=6.8 Hz, 6H).

Step 5. To a solution of tert-butyl 3-(4-(2-(2-aminopyridin-3-yl)-5-isopropyl-3H-imidazo[4,5-b]pyridin-3-yl)phenyl) azetidine-1-carboxylate (100 mg, 0.206 mmol) in DCM (5 mL) was added TFA (0.500 mL, 0.206 mmol). The mixture was stirred at 25° C. for 12 hrs. The reaction mixture was concentrated directly to give 3-(3-(4-(azetidin-3-yl)phenyl)-5-isopropyl-3H-imidazo[4,5-b]pyridin-2-yl)pyridin-2-amine (80.0 mg, 95% yield) as yellow oil, which was used in the next step directly. LCMS Rₜ=0.342 min in 1 min chromatography, purity 94.8%, MS ESI calcd, for 384.21, [M+H]⁺ 385.21, found 385.2.

Step 6. To a solution of 3-(3-(4-(azetidin-3-yl)phenyl)-5-isopropyl-3H-imidazo[4,5-b]pyridin-2-yl)pyridin-2-amine (80.0 mg, 0.208 mmol) and methyl 4-formylbenzoate (38.0 mg, 0.229 mmol) in DCM (10 mL) was added TEA (63.0 mg, 0.624 mmol) and NaBH(OAc)₃ (66.0 mg, 0.312 mmol). The mixture was stirred at 25° C. for 2 hrs. Water (5 mL) was added to the residue and the resulting mixture was extracted with DCM (3×5 mL). The combined organic phase was washed with brine (10 mL), dried over anhydrous Na₂SO₄, filtered and concentrated. The residue was purified by flash silica gel chromatography (eluent of 0~10% MeOH in DCM) to give methyl 4-((3-(4-(2-(2-aminopyridin-3-yl)-5-isopropyl-3H-imidazo[4,5-b]pyridin-3-yl)phenyl) azetidin-1-yl)methyl)benzoate (120 mg, 92% yield) as yellow oil. LCMS Rₜ=0.381 min in 1 min chromatography, purity 85.3%, MS ESI calcd, for 532.26. [M+H]⁺ 533.26, found 533.3

Step 7. To a solution of methyl 4-((3-(4-(2-(2-aminopyridin-3-yl)-5-isopropyl-3H-imidazo[4,5-b]pyridin-3-yl)phenyl) azetidin-1-yl)methyl)benzoate (100 mg, 0.188 mmol) in THF (3 mL) and Water (3 mL) was added LiOH·H₂O (12 mg, 0.282 mmol). The mixture was stirred at 25° C. for 2 hrs. The reaction mixture was concentrated directly. The residue was purified by prep-HPLC (Column: Welch Xtimate C18 150*30 mm*5 μm; mobile phase: [water (NH₄HCO₃)-ACN]; B %: 0%-35%; 25 min) to give 4-((3-(4-(2-(2-aminopyridin-3-yl)-5-isopropyl-3H-imidazo[4,5-b]pyridin-3-yl)phenyl) azetidin-1-yl)methyl)benzoic acid 173 (48 mg, 48.8% yield) as a white solid. ¹H NMR (DMSO-d₆, 400 MHz) δ_{H}=8.10 (d, J=8.4 Hz, 1H), 7.97 (dd, J=1.6, 4.8 Hz, 1H), 7.89 (d, J=8.0 Hz, 2H), 7.50 (d, J=8.4 Hz, 2H), 7.43 (d, J=8.0 Hz, 2H), 7.37 (d, J=8.4 Hz, 2H), 7.30 (d, J=8.0 Hz, 1H), 7.14 (dd, J=1.6, 7.6 Hz, 1H), 6.85 (s, 2H), 6.39 (dd, J=4.8, 7.6 Hz, 1H), 3.80-3.60 (s, 5H), 3.22 (t, J=6.4 Hz, 2H), 3.10-3.02 (m, 1H), 1.23 (d, J=6.8 Hz, 6H). HPLC Rₜ=1.674 min in 8 min chromatography, purity 99.91%. LCMS Rₜ=0.915 min in 2 min chromatography, purity 98.38%, MS ESI calcd. for 518.62, [M+H]⁺ 519.62, found 519.2.

### Example 143. Synthesis of 4((5-(2-(2-aminopyridin-3-yl)-6-(tert-butyl)-1H-benzo[d]imidazol-1-yl)pyridin-2-yl)carbamoyl)benzoic acid (Compound 174)

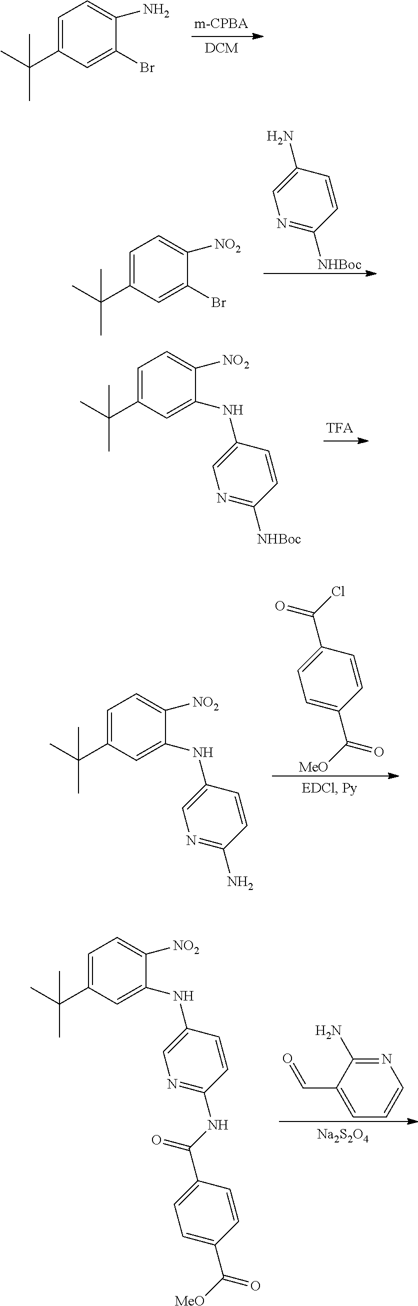

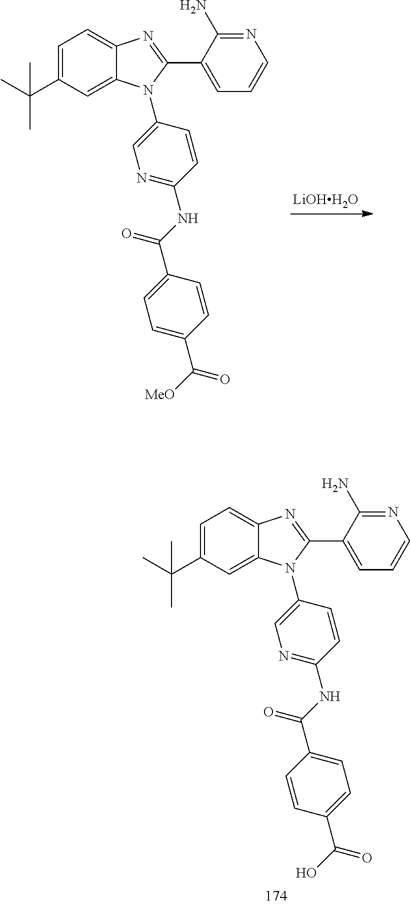 [SMILES: COC(=O)c1ccc(C(=O)Nc2ccc(-n3c(-c4cccnc4N)nc4ccc(C(C)(C)C)cc43)cn2)cc1 ; "LiOH·H2O" ; CC(C)(C)c1ccc2nc(-c3cccnc3N)n(-c3ccc(NC(=O)c4ccc(C(=O)O)cc4)nc3)c2c1]

Step 1. To a solution of 2-bromo-4-(tert-butyl) aniline (9.00 g, 39.5 mmol) in DCM (300 mL) was added m-CPBA (30.0 g, 148 mmol) at 25° C. The mixture was stirred at 70° C. for 12 hrs under N₂ atmosphere. The reaction was quenched with aqueous Na₂S₂O₃ (150 mL) and the resulting mixture was extracted with DCM (3×150 mL). The combined organic phase was washed with brine (100 mL), dried over anhydrous Na₂SO₄, filtered and concentrated. The residue was purified by combi-flash column (100% PE) to afford 2-bromo-4-(tert-butyl)-1-nitrobenzene (9.00 g, 88% yield) as yellow oil. ¹H NMR (DMSO-d₆, 400 MHz) δ_{H}=7.96 (d, J=8.4 Hz, 1H), 7.85 (d, J=2.0 Hz, 1H), 7.64 (dd, J=2.0, 8.4 Hz, 1H), 1.31 (s, 9H).

Step 2. To a solution of 2-bromo-4-(tert-butyl)-1-nitrobenzene (8.60 g, 33.3 mmol) in 1,4-Dioxane (100 mL) was added tert-butyl (5-aminopyridin-2-yl)carbamate (7.67 g, 36.7 mmol), Cs₂CO₃ (27.1 g, 83.3 mmol), Xantphos (1.45 g, 2.50 mmol) and Pd₂(dba)₃ (1.53 g, 1.67 mmol) at 25° C. The mixture was stirred at 90° C. for 12 hrs. The reaction mixture was filtered. The filtrate was dissolved in water (100 mL). The resulting suspension was extracted with DCM (3×200 mL). The combined organic phase was washed with brine (100 mL), dried over anhydrous Na₂SO₄, filtered and concentrated. The residue was purified by combi-flash column (EtOAc in PE, 0%~12%) to afford tert-butyl (5-((5-(tert-butyl)-2-nitrophenyl)amino)pyridin-2-yl)carbamate (12.2 g, 95% yield) as a red solid. ¹H NMR (DMSO-d₆, 400 MHz) δ_{H}=9.86 (s, 1H), 9.40 (s, 1H), 8.25 (d, J=2.4 Hz, 1H), 8.06 (d, J=8.8 Hz, 1H), 7.85 (d, J=8.8 Hz, 1H), 7.74 (dd, J=2.4, 8.8 Hz, 1H), 7.03-6.88 (m, 2H), 1.48 (s, 9H), 1.17 (s, 9H).

Step 3. A solution of tert-butyl (5-((5-(tert-butyl)-2-nitrophenyl)amino)pyridin-2-yl)carbamate (2.0 g, 5.18 mmol) in DCM (16 mL) and TFA (8.0 mL) was stirred at 25° C. for 1 hr. The mixture was concentrated directly to afford N₅-(5-(tert-butyl)-2-nitrophenyl)pyridine-2,5-diamine (1.80 g, crude) as a yellow solid. LCMS Rₜ=0.636 min in 1.5 min chromatography, purity 90%, MS ESI calcd, for 286.14 [M+H]⁺ 287.14, found 287.0

Step 4. To a solution of N₅-(5-(tert-butyl)-2-nitrophenyl)pyridine-2,5-diamine (1.75 g, 6.11 mmol) in Pyridine (20 mL) was added methyl 4-(chlorocarbonyl)benzoate (1.46 g, 7.33 mmol) and EDCI (3.51 g, 18.3 mmol) at 25° C. The mixture was heated to 80° C. and stirred for 2 hrs. then diluted with EtOAc (50 mL), washed with aqueous HCl (4×10 mL, 1M). The combined organic phase was washed with brine (2×20 mL), dried over anhydrous Na₂SO₄, filtered and concentrated. The residue was purified by combi-flash column (EtOAc in PE, 0%~20%) to afford methyl 4-((5-((5-(tert-butyl)-2-nitrophenyl)amino)pyridin-2-yl)carbamoyl)benzoate (570 mg, 1.27 mmol, 21% yield) as a yellow solid. JH NMR (DMSO-d₆, 400 MHz) δ_{H}=11.09 (s, 1H), 9.46 (s, 1H), 8.42 (d, J=2.4 Hz, 1H), 8.25 (d, J=8.8 Hz, 1H), 8.18-8.11 (m, 2H), 8.11-8.04 (m, 3H), 7.87 (dd, J=2.8, 8.8 Hz, 1H), 7.10 (s, 1H), 6.99 (dd, J=1.6, 8.8 Hz, 1H), 3.90 (s, 3H), 1.20 (s, 9H).

Step 5. To a solution of methyl 4-((5-((5-(tert-butyl)-2-nitrophenyl)amino)pyridin-2-yl)carbamoyl)benzoate (520 mg, 1.16 mmol) in DMSO (6 mL) and methanol (6 mL) was added 2-aminonicotinaldehyde (212 mg, 1.74 mmol) and Na₂S₂O₄ (1.01 g, 5.80 mmol) at 25° C. The mixture was heated to 100° C. and stirred for 12 hrs. The mixture was diluted with EtOAc (20 mL), washed with brine (3×10 mL). The combined organic phase was dried over anhydrous Na₂SO₄, filtered and concentrated. The residue was purified by combi-flash column (EtOAC in PE, 0%~60%) to afford methyl 4-((5-(2-(2-aminopyridin-3-yl)-6-(tert-butyl)-1H-benzo[d]imidazol-1-yl)pyridin-2-yl)carbamoyl)benzoate (330 mg, 55% yield) as a yellow solid. ¹H NMR (DMSO-d₆, 400 MHz) δ=11.29 (s, 1H), 8.47-8.32 (m, 2H), 8.18-8.02 (m, 5H), 7.98 (dd, J=1.6, 4.8 Hz, 1H), 7.75 (d, J=8.4 Hz, 1H), 7.45 (dd, J=1.6, 8.8 Hz, 1H), 7.25 (dd, J=1.6, 7.6 Hz, 1H), 7.17 (s, 1H), 6.81 (s, 2H), 6.46 (dd, J=4.8, 7.6 Hz, 1H), 3.90 (s, 3H), 1.31 (s, 9H).

Step 6. A solution of methyl 4-((5-(2-(2-aminopyridin-3-yl)-6-(tert-butyl)-1H-benzo[d]imidazol-1-yl)pyridin-2-yl)carbamoyl)benzoate (100 mg, 0.190 mmol) and LiOH·H₂O (24.0 mg, 0.580 mmol) in THF (2 mL) and water (2 mL) was stirred at 25° C. for 12 hrs. The mixture was filtered directly. The mixture was purified by prep-HPLC (column: Xtimate C18 200*40 mm*7 μm: mobile phase: [water (HCl)-ACN]; B %: 12%-52%, 20.5 min) to afford 4-((5-(2-(2-aminopyridin-3-yl)-6-(tert-butyl)-1H-benzo[d]imidazol-1-yl)pyridin-2-yl)carbamoyl)benzoic acid 174 (60.3 mg, 62% yield) as a white solid. ¹H NMR (DMSO-d₆, 400 MHz) δ=11.28 (s, 1H), 8.53 (d, J=2.4 Hz, 1H), 8.41 (d, J=8.8 Hz, 1H), 8.13-8.05 (m, 6H), 7.89-7.77 (m, 2H), 7.54 (dd, J=1.6, 8.8 Hz, 1H), 7.24 (d, J=1.2 Hz, 1H), 6.87 (t, J=6.4 Hz, 1H), 1.33 (s, 9H). HPLC Rₜ=1.995 min in 8 min chromatography, purity 99.9%. LCMS Rₜ=1.915 min in 4 min chromatography, purity 98.6%, MS ESI calcd, for $06.21 [M+H]⁺ 507.21, found 507.2.

### Example 144. Synthesis of 4-((5-(2-(2-aminopyridin-3-yl)-6-(trifluoromethoxy)-1H-benzo[d]imidazol-1-yl)pyridin-2-yl)carbamoyl)benzoic acid (Compound 175)

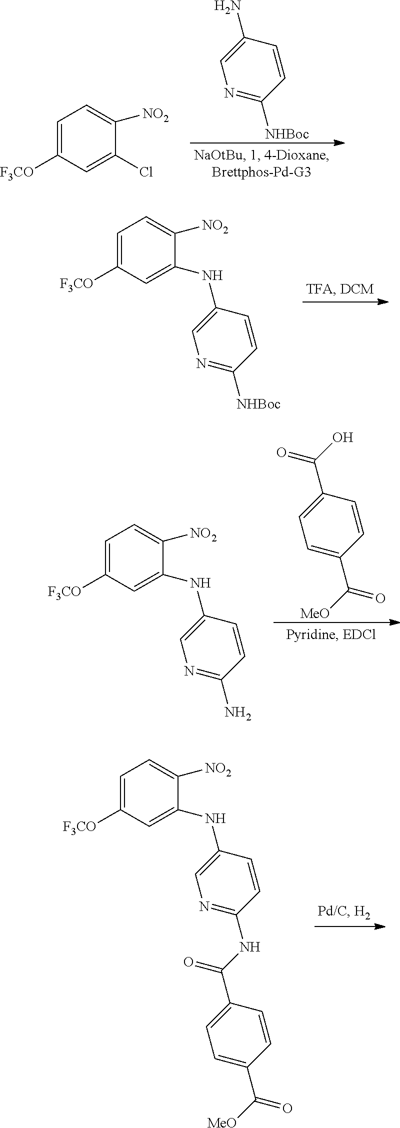

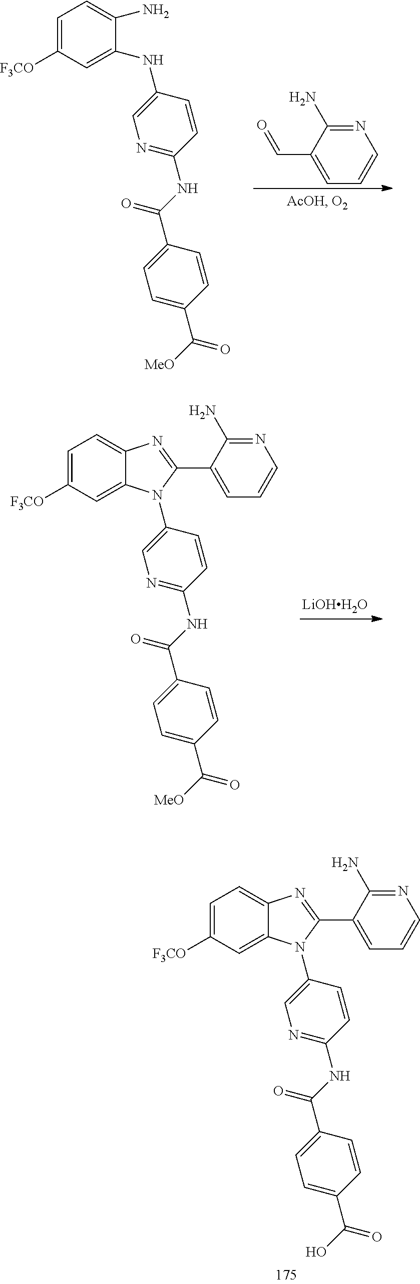

Step 1. To a solution of tert-butyl (5-aminopyridin-2-yl)carbamate (2.43 g, 11.6 mmol) in 1,4-Dioxane (60.0 mL) was added 2-chloro-1-nitro-4-(trifluoromethoxy)benzene (2.00 g, 8.28 mmol), NaOtBu (2.39 g, 24.8 mmol) and Brettphos-Pd-G3 (750 mg, 0.828 mmol) at 25° C. The reaction mixture was degassed and purged with N₂ for 3 times then heated to 80° C. and stirred for 14 hrs under N₂. Water (60 mL) was added to the reaction and the resulting mixture was extracted with EtOAc (3×60 mL). The combined organic phase was washed with brine (50 mL), dried over anhydrous Na₂SO₄, filtered and concentrated. The residue was purified by flash silica gel chromatography (eluent of 0~5% EtOAc in PE) to give tert-butyl (5-((2-nitro-5-(trifluoromethoxy)phenyl)amino)pyridin-2-yl)carbamate (1.60 g, 47% yield) as a yellow solid. ¹H NMR (DMSO-d₆, 400 MHz) δ_{H}=9.90 (s, 1H), 9.54 (s, 1H), 8.32-8.20 (m, 2H), 7.87 (d, J=8.8 Hz, 1H), 7.73 (dd, J=8.8, 2.4 Hz, 1H), 6.80 (d, J=9.2 Hz, 1H), 6.70 (s, 1H), 1.48 (s, 9H).

Step 2. To a solution of tert-butyl (5-((2-nitro-5-(trifluoromethoxy)phenyl)amino)pyridin-2-yl)carbamate (500 mg, 1.20 mmol) in DCM (10 mL) was added TFA (137 mg, 1.20 mmol). The mixture was stirred at 25° C. for 12 hrs. The reaction mixture was concentrated directly to give N5-(2-nitro-5-(trifluoromethoxy)phenyl)pyridine-2,5-diamine (400 mg, 95% yield) as yellow oil, which was used in the next step directly. LCMS Rₜ=0.391 min in 1 min chromatography, purity 90.5%, MS ESI calcd. for 314.22 [M+H]⁺ 315.22, found 315.00.

Step 3. To a solution of N₅-(2-nitro-5~ (trifluoromethoxy)phenyl)pyridine-2,5-diamine (350 mg, 1.11 mmol) and 4-(methoxycarbonyl)benzoic acid (201 mg, 1.11 mmol) in Pyridine (10 mL) was added EDCI (320 mg, 1.67 mmol). The mixture was stirred at 25° C. for 12 hrs. then water (5 mL) was added to the residue and the resulting mixture was extracted with EtOAc (3×10 mL). The combined organic phase was washed with brine (10 mL), dried over anhydrous Na₂SO₄, filtered and concentrated. The residue was purified by flash silica gel chromatography (eluent of 0~20% EtOAc in PE) to give methyl 4-((5-((2-nitro-5-(trifluoromethoxy)phenyl)amino)pyridin-2-yl)carbamoyl)benzoate (410 mg, 77% yield) as light yellow oil. ¹H NMR (DMSO-d₆, 400 MHz) δ_{H}=11.15 (s, 1H), 9.62 (s, 1H), 8.41 (d, J=2.4 Hz, 1H), 8.28 (dd, J=9.2, 5.2 Hz, 2H), 8.16-8.12 (m, 2H), 8.08 (s, 1H), 7.87 (dd, J=8.8, 2.4 Hz, 1H), 7.28-7.17 (m, 3H), 3.90 (s, 3H).

Step 4. To a solution of methyl 4-((5-((2-nitro-5-(trifluoromethoxy)phenyl)amino)pyridin-2-yl)carbamoyl)benzoate (400 mg, 0.840 mmol) in THF (10 mL) was added Pd/C (100 mg, 10%) under N₂. The reaction mixture was degassed and purged with H₂ (15 psi) for 3 times and then stirred at 25° C. for 12 hrs under H₂ (15 psi). The mixture was filtered and the filtrate was concentrated directly to give methyl 4-((5-((2-amino-5-(trifluoromethoxy)phenyl)amino)pyridin-2-yl)carbamoy ll)benzoate (260 mg, 69% yield) as a yellow solid, which was used in the next step directly. ¹H NMR (DMSO-do, 400 MHz) δ_{H}=10.77 (s, 1H), 8.13-8.09 (m, 2H), 8.05-8.03 (m, 2H), 8.02-7.95 (m, 2H), 7.48 (s, 1H), 7.26 (dd, J=2.8, 8.8 Hz, 1H), 6.91 (s, 1H), 6.81-6.78 (m, 1H), 3.91-3.89 (m, 3H).

Step 5. To solution of methyl 4-((5-((2-amino-5-(trifluoromethoxy)phenyl)amino)pyridin-2-yl)carbamoyl)benzoate (260 mg, 0.582 mmol) in acetic acid (5 mL) was added 2-aminonicotinaldehyde (85.0 mg, 0.699 mmol) under 02. The mixture was heated to 80° C. and stirred for 1 hr under 02. The reaction mixture was concentrated directly and the residue was purified by flash silica gel chromatography (eluent of 60-70% EtOAc in PE) to give methyl 4-((5-(2-(2-aminopyridin-3-yl)-6-(trifluoromethoxy)-1H-benzo[d]imidazol-1-yl)pyridin-2-yl)carbamoyl)benzoate (200 mg, 63% yield) as light yellow oil. ¹H NMR (DMSO-d₆, 400 MHz) δ_{H}=11.30 (s, 1H), 8.45 (d, J=2.4 Hz, 1H), 8.37 (d, J=8.8 Hz, 1H), 8.17-8.12 (m, 2H), 8.10-8.06 (m, 3H), 8.02-7.99 (m, 1H), 7.93 (d, J=8.8 Hz, 1H), 7.38-7.29 (m, 3H), 6.73 (s, 2H), 6.49 (dd, J=7.6, 4.8 Hz, 1H), 3.90 (s, 3H).

Step 6. To a solution of methyl 4-((5-(2-(2-aminopyridin-3-yl)-6-(trifluoromethoxy)-1H-benzo[d]imidazol-1-yl)pyridin-2-yl)carbamoyl)benzoate (200 mg, 0.365 mmol) in THF (4 mL) and water (4 mL) was added LiOH·H₂O (46.0 mg, 1.09 mmol). The mixture was heated to 40° C. and stirred for 2 hrs. The reaction mixture was concentrated directly and the residue was purified by prep-HPLC (Column: Welch Xtimate C18 150*30 mm*5 μm; mobile phase: [water (NH₄HCO₃)-ACN]: B %: 6%-46%; 25 min) to give 4-((5-(2-(2-aminopyridin-3-yl)-6-(trifluoromethoxy)-1H-benzo[d]imidazol-1-yl)pyridin-2-yl)carbamoyl)benzoic acid 175 (78.6 mg, 40% yield) as a light yellow solid. ¹H NMR (DMSO-d₆, 400 MHz) δ=11.22 (s, 1H), 8.51-8.30 (m, 2H), 8.11-7.99 (m, 6H), 7.92 (d, J=8.4 Hz, 1H), 7.40-7.26 (m, 3H), 6.73 (s, 2H), 6.52-6.46 (m, 1H), HPLC Rₜ=2.58 min in 8 min chromatography, purity 100%. LCMS Rₜ=1.238 min in 2 min chromatography, purity 99.4%, MS ESI calcd, for 534.13, [M+H]⁺ 535.13, found 535.0.

### Example 145. Synthesis of 4-[[5-[2-(2-amino-3-pyridyl)-6-isopropoxy-benzimidazol-1-yl]-2-pyridyl]carbamoyl]benzoic acid (Compound 176)

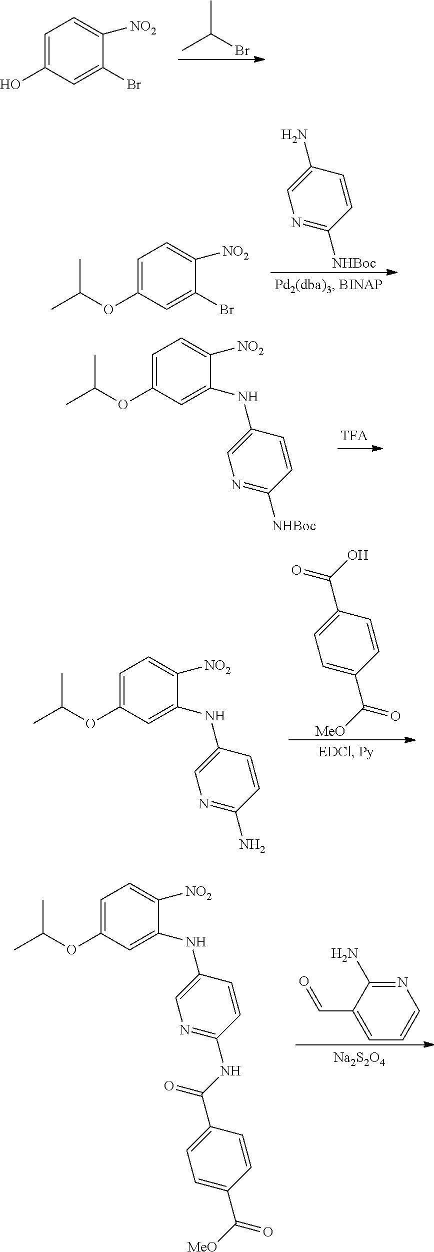

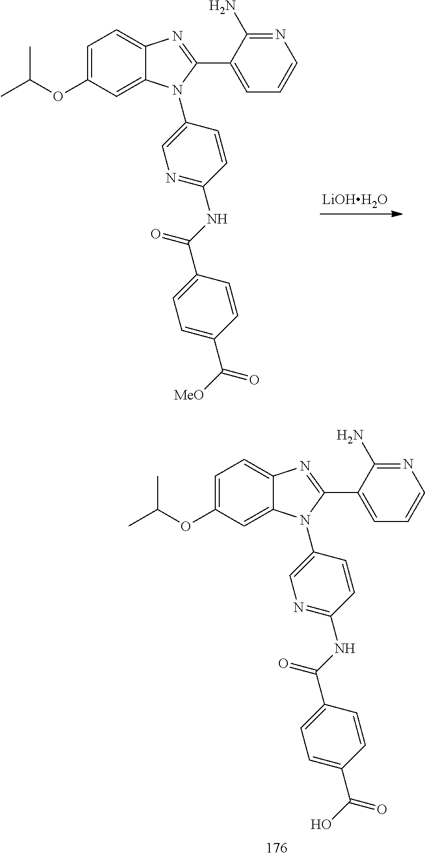 [SMILES: COC(=O)c1ccc(C(=O)Nc2ccc(-n3c(-c4cccnc4N)nc4ccc(OC(C)C)cc43)cn2)cc1 ; "LiOH·H2O" ; CC(C)Oc1ccc2nc(-c3cccnc3N)n(-c3ccc(NC(=O)c4ccc(C(=O)O)cc4)nc3)c2c1]

Step 1. To a solution of 3-bromo-4-nitro-phenol (2.00 g, 9.17 mmol) in DMF (30 mL) was added K₂CO₃ (3.80 g, 27.5 mmol) and 2-bromopropane (2.26 g, 18.3 mmol). The reaction was heated to 90° C. and stirred for 16 hrs. Water (100 mL) was then added and the mixture was extracted with EtOAc (3×100 mL). The organic layer was dried over Na₂SO₄ and concentrated in vacuo. The crude was purified by flash column (0~50% of EtOAc in PE) to give 2-bromo-4-isopropoxy-1-nitro-benzene (2.00 g, 84% yield) as yellow oil. ¹H NMR (400 MHz, DMSO-d₆) δ=8.04 (d, J=9.2 Hz, 1H), 7.40 (d, J=2.8 Hz, 1H), 7.12 (dd, J=2.8, 8.8 Hz, 1H), 4.90-4.73 (m, 1H), 1.29 (d, J=6.4 Hz, 6H).

Step 2. To the solution of 2-bromo-4-isopropoxy-1-nitro-benzene (2.30 g, 8.84 mmol), t-BuONa (2.55 g, 26.5 mmol) and BINAP (700 mg, 1.13 mmol) in toluene (50 mL) was added Pd₂(dba)₃ (460 mg, 0.800 mmol), the reaction was heated to 80° C. and stirred for 1 hr. The reaction mixture was concentrated and diluted with EtOAc (100 mL) and washed with water (50 mL) and brine (50 mL×2), dried over anhydrous sodium sulfate, filtered and concentrated. The crude product was purified by flash chromatography on silica gel (20% of EtOAc in PE) to afford tert-butyl N-[5-(5-isopropoxy-2-nitro-anilino)-2-pyridyl]carbamate (1.00 g. 87% yield) as a yellow solid. ¹H NMR (400 MHz, DMSO-d₆) δ=9.89 (s, 1H), 9.53 (s, 1H), 8.24 (d, J=2.4 Hz, 1H), 8.10 (d, J=9.6 Hz, 1H), 7.86 (d, J=9.2 Hz, 1H), 7.75 (dd, J=2.4, 8.8 Hz, 1H), 6.46 (dd, J=2.4, 9.6 Hz, 1H), 6.19 (d, J=2.4 Hz, 1H), 4.55 (q, J=6.0 Hz, 1H), 1.48 (s, 9H), 1.21 (d, J=6.0 Hz, 6H).

Step 3. To a solution of tert-butyl N-[5-(5-isopropoxy-2-nitro-anilino)-2-pyridyl]carbamate (1.20 g, 3.09 mmol) in DCM (6 mL) was added TFA (6 mL, 3.09 mmol) at 25° C. The mixture was stirred at 25° C. for 5 hr then concentrated in vacuo to give N₅-(5-isopropoxy-2-nitro-phenyl)pyridine-2.5-diamine (1.50 g, crude) as a yellow solid, which was directly used in the next step. ¹H NMR (400 MHz, DMSO-d₆) δ=9.38 (s, 1H), 8.27-8.14 (m, 1H), 8.11 (d, J=9.6 Hz, 1H), 8.00 (s, 1H), 7.94 (dd, J=2.0, 9.2 Hz, 1H), 7.03 (d, J=9.2 Hz, 1H), 6.50 (dd, J=2.4, 9.2 Hz, 1H), 6.20 (d, J=2.4 Hz, 1H), 4.66 (q, J=6.0 Hz, 1H), 1.22 (d, J=6.0 Hz, 6H).

Step 4. To a solution of N₅-(5-isopropoxy-2-nitro-phenyl)pyridine-2,5-diamine (350 mg, 1.21 mmol) in Pyridine (10 mL) was added 4-methoxycarbonylbenzoic acid (262 mg, 1.46 mmol) and EDCI (348 mg, 1.82 mmol) at 25° C. under N₂. The mixture was stirred at 25° C. for 16 hrs. then water (50 mL) was added and the mixture was extracted with EtOAc (3×50 mL). The organic phase was washed with HCl (50 mL) and brine (50 mL), dried over Na₂SO₄, filtered and concentrated in vacuo. The crude was purified by flash chromatography eluting with PE/EtOAc (from 0% to 25% of EtOAc in PE) to give methyl 4-[[5-(5-isopropoxy-2-nitro-anilino)-2-pyridyl]carbamoyl]benzoate (155 mg, 28% yield) as a yellow solid. ¹H NMR (400 MHz, DMSO-d₆) δ=11.13 (s, 1H), 9.61 (s, 1H), 8.42 (d, J=2.4 Hz, 1H), 8.27 (d, J=8.8 Hz, 1H), 8.17-8.11 (m, 3H), 8.07 (d, J=9.6 Hz, 3H), 7.90 (dd, J=2.4, 8.8 Hz, 1H), 6.49 (dd, J=2.4, 9.6 Hz, 1H), 6.32 (d, J=2.4 Hz, 1H), 4.63-4.54 (m, 1H), 3.90 (s, 3H), 1.22 (d, J=6.0 Hz, 6H).

Step 5. To a solution of methyl 4-[[5-(5-isopropoxy-2-nitro-anilino)-2-pyridyl]carbamoyl]benzoate (155 mg, 0.344 mmol) in DMSO (4.00 mL) and methanol (1.00 mL) was added 2-aminopyridine-3-carbaldehyde (101 mg, 0.826 mmol) and Na₂S₂O₄ (300 mg, 1.72 mmol) at 25° C. The mixture was stirred at 100° C. for 32 hrs, the water (20 mL) was added. The mixture was extracted with EtOAc (3×20 mL) and washed with brine (20 mL), dried over Na₂SO₄, filtered and concentrated in vacuo. The crude was purified by flash chromatography eluting with PE/EtOAc (from 0% to 30% of EtOAc in PE) to give methyl 4-[[5-[2-(2-amino-3-pyridyl)-6-isopropoxy-benzimidazol-1-yl]-2-pyridyl]carbamoyl]benzoate (50.0 mg, 28% yield) as yellow oil. ¹H NMR (400 MHz, DMSO-d₆) δ=8.46-8.35 (m, 3H), 8.19-8.01 (m, 8H), 7.97 (dd, J=1.6, 4.8 Hz, 1H), 7.71-7.63 (m, 1H), 7.21 (dd, J=1.6, 7.6 Hz, 1H), 6.97-6.93 (m, 1H), 6.85 (s, 2H), 6.73 (d, J=2.4 Hz, 1H), 6.45 (dd, J=5.2, 7.6 Hz, 1H).

Step 6. To a solution of methyl 4-[[5-[2-(2-amino-3-pyridyl)-6-isopropoxy-benzimidazol-1-yl]-2-pyridyl]carbamoyl]benzoate (50.0 mg, 0.0957 mmol) in THF (1 mL) and water (1 mL) was added LiOH·H₂O (10.0 mg, 0.239 mmol) at 25° C. The mixture was stirred at 25° C. for 16 hrs. then concentrated in vacuo. The crude was purified by prep-HPLC (Welch Xtimate C18 150*30 mm*5 μm water (FA)-ACN) to give 4-[[5-[2-(2-amino-3-pyridyl)-6-isopropoxy-benzimidazol-1-yl]-2-pyridyl]carbamoyl]benzoic acid 176 (8.60 mg, 18% yield) as a yellow solid. ¹H NMR (400 MHz, DMSO-d₆) δ=8.40-8.36 (m, 2H), 8.08-8.01 (m, 5H), 7.97 (dd, J=1.6, 5.2 Hz, 1H), 7.69 (d, J=8.8 Hz, 1H), 7.21 (dd, J=2.0, 7.6 Hz, 1H), 6.95 (dd, J=2.4, 8.8 Hz, 1H), 6.86 (s, 2H), 6.73 (d, J=2.4 Hz, 1H), 6.45 (dd, J=4.8, 7.6 Hz, 1H), 4.60 (td, J=6.0, 12.0 Hz, 1H), 1.24 (d, J=6.0 Hz, 6H). HPLC Rₜ=2.926 min in 8.0 min chromatography, 10-80AB_8 min.1 cm, purity 100%. LCMS Rₜ==2.468 min in 4.0 min chromatography, purity 97.0%, MS ESI calcd, for 508.19 [M+H]⁺ 509.19, found 509.1.

### Example 146. Synthesis of 4-(5-(2-(2-aminopyridin-3-yl)-5-phenyl-3H-imidazo[4,5-b]pyridin-3-yl)picolinamido)-2-methylbenzoic acid (Compound 177)

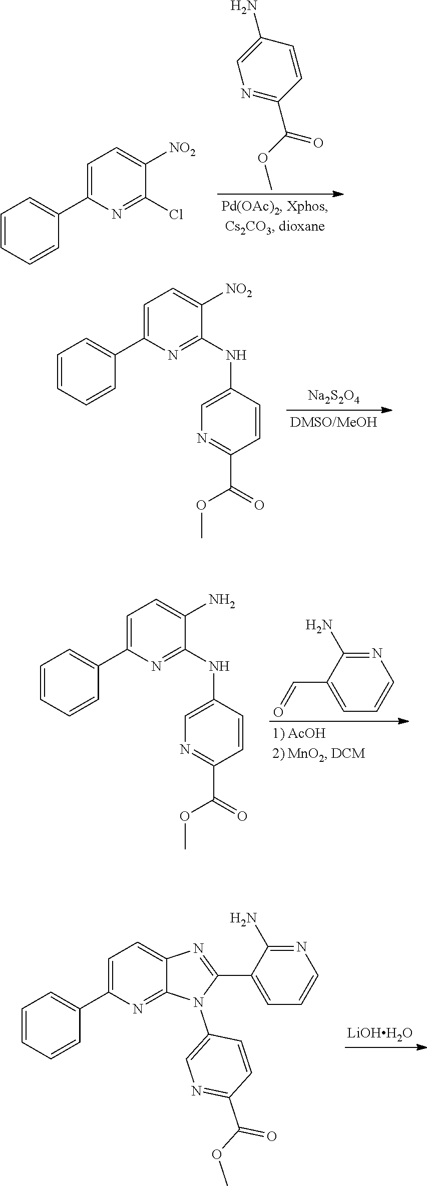

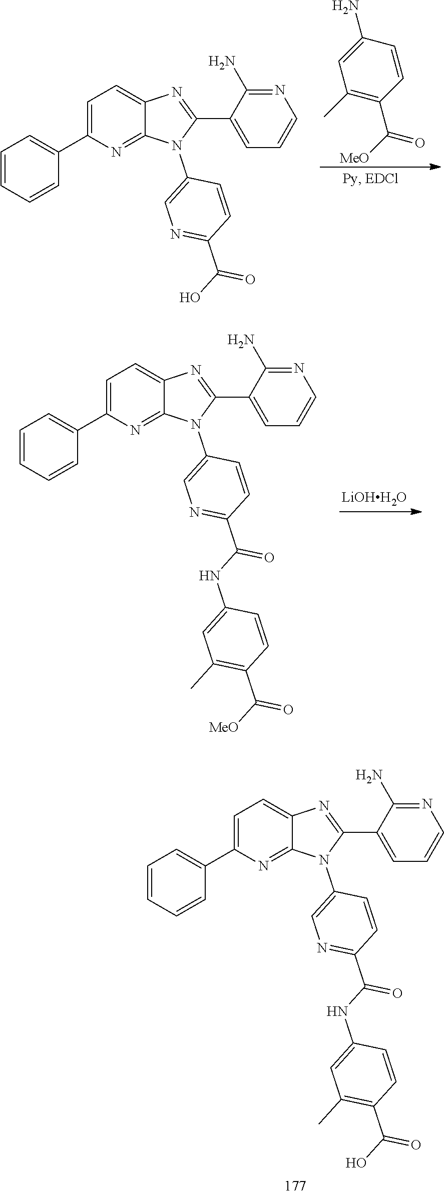

Step 1. To a solution of 2-chloro-3-nitro-6-phenyl-pyridine (10.0 g, 42.6 mmol) and methyl 5-aminopyridine-2-carboxylate (6.48 g, 42.6 mmol) in 1,4-Dioxane (150 mL) was added Cs₂CO₃ (41.7 g, 128 mmol), Xphos (4.06 g, 8.52 mmol) and Pd(OAc)₂ (0.96 g, 4.26 mmol) under N₂. The reaction mixture was degassed and purged with N₂ for 3 times. The mixture was heated to 100° C. and stirred for 12 hrs under N₂. Water (50 mL) was added to the reaction mixture and the resulting mixture was extracted with EtOAc (3×50 mL). The combined organic phase was washed with brine (50 mL), dried over anhydrous Na₂SO₄, filtered and concentrated. The residue was purified by flash silica gel chromatography (eluent of 0~12% MeOH in DCM) to give methyl 5-((3-nitro-6-phenylpyridin-2-yl)amino) picolinateS (6.80 g, 43% yield) as a yellow solid. ¹H NMR (DMSO-d₆, 400 MHz) &H=10.31 (s, 1H), 9.11 (d, J=2.4 Hz, 1H), 8.66 (d, J=8.8 Hz, 1H), 8.40 (dd, J=2.4, 8.4 Hz, 1H), 8.16-8.09 (m, 3H), 7.73 (d, J=8.4 Hz, 1H), 7.58-7.55 (m, 3H), 3.89 (s, 3H).

Step 2. To a solution of methyl 5-((3-nitro-6-phenylpyridin-2-yl)amino) picolinate (5.60 g, 16.0 mmol) in DMSO (40 mL) and methanol (40 mL) was added Na₂S₂O₄ (5.57 g, 32.0 mmol) under N₂. The mixture was heated to 100° C. and stirred for 12 hrs. then concentrated. Saturated aq. LiCl (30 mL) was added to the residue and the resulting mixture was extracted with EtOAc (3×30 mL). The combined organic phase was washed with brine (30 mL), dried over anhydrous Na₂SO₄, filtered and concentrated to give Methyl 5-((3-amino-6-phenylpyridin-2-yl)amino) picolinate (2.80 g, crude) as light yellow oil, which was used in the next step directly. ¹H NMR (DMSO-d₆, 400 MHz) δ_{H}=9.02 (d, J=2.4 Hz, 1H), 8.59 (s, 1H), 8.30 (dd, J=2.4, 8.8 Hz, 1H), 8.02 (d, J=8.8 Hz, 1H), 7.94-7.91 (m, 2H), 7.76-7.72 (m, 1H), 7.43-7.41 (m, 2H), 7.10 (d, J=8.0 Hz, 1H), 6.94-6.91 (m, 1H), 5.40 (s, 2H), 3.84 (s, 3H).

Step 3. To a solution of methyl 5-((3-amino-6-phenylpyridin-2-yl)amino) picolinate (2.50 g. 7.80 mmol) in acetic acid (20 mL) was added 2-aminopyridine-3-carbaldehyde (1.05 g. 8.58 mmol). The mixture was heated to 110° C. and stirred for 1 hr. then concentrated. DCM (40 mL) and MnO₂ (6.80 g, 78 mmol) were added and the resulting mixture was stirred at 50° C. for 16 hrs. The reaction mixture was filtered and the filtrate was concentrated to dryness. The residue was purified by flash silica gel chromatography (eluent of 0~10% MeOH in DCM) to give methyl 5-(2-(2-aminopyridin-3-yl)-5-phenyl-3H-imidazo[4,5-b]pyridin-3-yl)picolinate (1.50 g, 40% yield) as yellow oil. ¹H NMR (DMSO-d₆, 400 MHz) δ_{H}=8.89 (d, J=2.0 Hz, 1H), 8.32 (d, J=8.4 Hz, 1H), 8.24 (d, J=8.4 Hz, 1H), 8.16-8.13 (m, 1H), 8.09-8.07 (m, 2H), 8.06-8.03 (m, 2H), 7.49-7.46 (m, 2H), 7.42-7.38 (m, 2H), 6.67 (s, 2H), 6.54 (dd, J=4.8, 7.6 Hz, 1H). 3.92 (s, 3H).

Step 4. To a solution of methyl 5-(2-(2-aminopyridin-3-yl)-5-phenyl-3H-imidazo[4,5-b]pyridin-3-yl)picolinate (1.50 g, 3.55 mmol) in water (20 mL) and THF (20 mL) was added LiOH·H₂O (0.75 g, 17.8 mmol). The mixture was stirred at 25° C. for 12 hrs. then concentrated directly. The residue was purified by prep-HPLC (Column: Welch Xtimate C18 150*30 mm*Sum; mobile phase: [water (HCl)-ACN]; B %: 0%-35%; 25 min) to give 5-(2-(2-aminopyridin-3-yl)-5-phenyl-3H-imidazo[4,5-b]pyridin-3-yl)picolinic acid (400 mg, 28% yield) as a light yellow solid. ¹H NMR (DMSO-d₆, 400 MHz) δ_{H}=8.94 (d, J=2.0 Hz, 1H), 8.40 (d, J=8.4 Hz, 1H), 8.29-7.87 (m, 8H), 7.52-7.41 (m, 3H), 6.93-6.86 (m, 1H).

Step 5. To a solution of 5-(2-(2-aminopyridin-3-yl)-5-phenyl-3H-imidazo[4,5-b]pyridin-3-yl)picolinic acid (200 mg, 0.490 mmol) and methyl 4-amino-2-methyl-benzoate (81 mg, 0.490 mmol) in Pyridine (10 mL) was added EDCI (140 mg, 0.735 mmol). The mixture was stirred at 25° C. for 12 hrs. Water (5 mL) was added to the residue and the resulting mixture was extracted with EtOAc (3×10 mL). The combined organic phase was washed with brine (10 mL), dried over anhydrous Na₂SO₄, filtered and concentrated. The residue was purified by flash silica gel chromatography (eluent of 0~12% MeOH in DCM) to give methyl 4-(5-(2-(2-aminopyridin-3-yl)-5-phenyl-3H-imidazo[4,5-b]pyridin-3-yl)picolinamido)-2-methylbenzoate (200 mg, 74% yield) as light yellow oil. ¹H NMR (400 MHz, DMSO-d₆) δ=10.93 (s, 1H), 8.91 (d, J=2.0 Hz, 1H), 8.37-8.23 (m, 3H), 8.11-8.05 (m, 4H), 7.95-7.86 (m, 3H), 7.51-7.39 (m, 4H), 6.67 (s, 2H), 6.56 (dd, J=4.8, 7.6 Hz, 1H), 3.81 (s, 3H), 2.54 (s, 3H).

Step 6. To a solution of methyl 4-(5-(2-(2-aminopyridin-3-yl)-5-phenyl-3H-imidazo[4,5-b]pyridin-3-yl)picolinamido)-2-methylbenzoate (180 mg, 0.324 mmol) in THF (3 mL), water (3 mL) and methanol (3 mL) was added NaOH (817 mg, 19.4 mmol). The mixture was heated to 50° C. and stirred for 2 hrs. then concentrated directly. The residue was purified by prep-HPLC (Column: Welch Xtimate C18 150*30 mm*5 μm; mobile phase: [water (FA)-ACN]; B %: 0%-35%; 25 min) to give 4-(5-(2-(2-aminopyridin-3-yl)-5-phenyl-3H-imidazo[4,5-b]pyridin-3-yl)picolinamido)-2-methylbenzoic acid 177 (3.30 mg, 2% yield) as a light yellow solid. ¹H NMR (400 MHz, DMSO-d₆) δ=10.86 (s, 1H), 8.91 (s, 1H), 8.37-8.23 (m, 3H), 8.12-8.04 (m, 4H), 7.90-7.80 (m, 3H), 7.52-7.39 (m, 4H), 6.68 (s, 2H), 6.56 (dd, J=5.6, 7.2 Hz, 1H), 2.54 (s, 3H). HPLC Rₜ=2.948 min in 8 min chromatography, purity 100%. LCMS Rₜ=1.311 min in 2 min chromatography, purity 99.6%, MS ESI calcd. for 541.19 [M+H]⁺ 542.19, found 542.1.

### Example 147. Synthesis of 4-(5-(2-(2-aminopyridin-3-yl)-5-phenyl-3H-imidazo[4,5-b]pyridin-3-yl)picolinamido)-3-methylbenzoic acid (Compound 178)

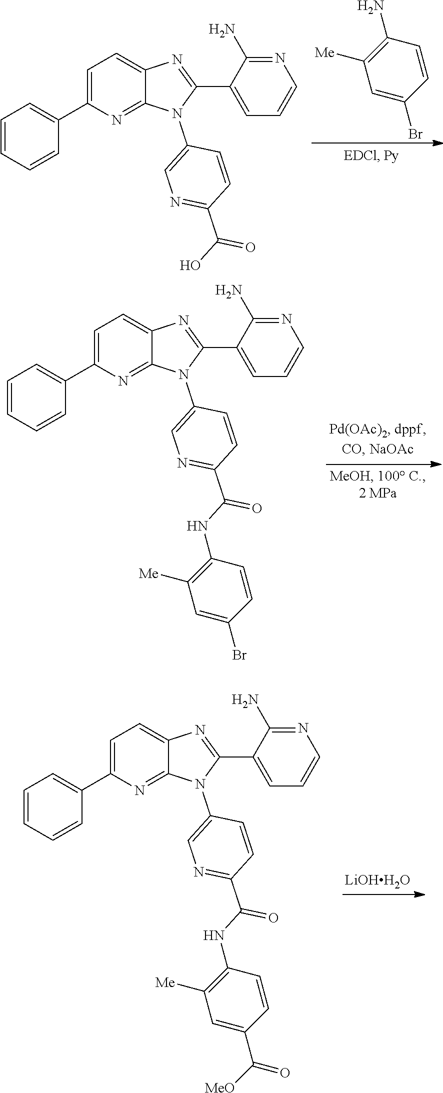

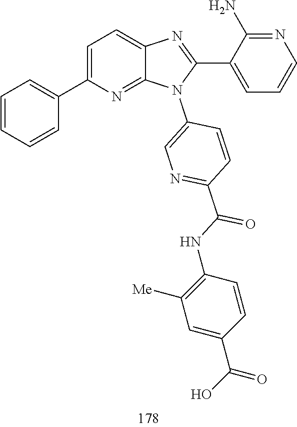 [SMILES: Cc1cc(C(=O)O)ccc1NC(=O)c1ccc(-n2c(-c3cccnc3N)nc3ccc(-c4ccccc4)nc32)cn1]

Step 1. To a solution of 5-(2-(2-aminopyridin-3-yl)-5-phenyl-3H-imidazo[4,5-b]pyridin-3-yl)picolinic acid (350 mg, 0.857 mmol) and 4-bromo-2-methyl-aniline (159 mg, 0.857 mmol) in Pyridine (5 mL) was added EDCI (246 mg, 1.29 mmol). The mixture was stirred at 25° C. for 12 hrs. then water (5 mL) was added to the reaction mixture. The resulting mixture was extracted with EtOAc (3×10 mL) and the combined organic phase was washed with brine (10 mL), dried over anhydrous Na₂SO₄, filtered and concentrated. The residue was purified by flash silica gel chromatography (eluent of 0~12% MeOH in DCM) to give 5-(2-(2-aminopyridin-3-yl)-5-phenyl-3H-imidazo[4,5-b]pyridin-3-yl)-N-(4-bromo-2-methylphenyl) picolinamide (420 mg, 77% yield) as light yellow oil. ¹H NMR (DMSO-d₆, 400 MHz) δ_{H}=10.38 (s, 1H), 8.96-8.85 (m, 1H), 8.36-8.21 (m, 3H), 8.10-8.04 (m, 4H), 7.67 (d, J=8.4 Hz, 1H), 7.53-7.39 (m, 6H), 6.67 (s, 2H), 6.56 (dd, J=4.8, 7.6 Hz, 1H), 2.30 (s, 3H).

Step 2. To a solution of 5-(2-(2-aminopyridin-3-yl)-5-phenyl-3H-imidazo[4,5-b]pyridin-3-yl)-N-(4-bromo-2-methylphenyl)picolinamide (420 mg, 0.729 mmol) and sodium acetate (179 mg, 2.19 mmol) in methanol (5 mL) and DMSO (10 mL) was added dppf (40.0 mg, 0.0729 mmol) and Pd(OAc)₂ (25.0 mg, 0.109 mmol) at 25° C. The reaction mixture was degassed and purged with CO for 3 times then heated to 100° C. and stirred for 12 hrs under CO (2 MPa). The reaction mixture was concentrated, and water (1 mL) was added to the residue. The resulting mixture was extracted with EtOAc (3×20 mL) and the combined organic phase was washed with brine (10 mL), dried over anhydrous Na₂SO₄, filtered and concentrated. The residue was purified by flash silica gel chromatography (eluent of 0~10% MeOH in DCM) to give methyl 4-(5-(2-(2-aminopyridin-3-yl)-5-phenyl-3H-imidazo[4,5-b]pyridin-3-yl)picolinamido)-3-methylbenzoate [3.3]heptane-2-carboxy late (400 mg, 83% yield) as yellow oil. ¹H NMR (DMSO-d₆, 400 MHz) δ_{H}=10.47 (s, 1H), 8.97-8.87 (m, 1H), 8.39-8.30 (m, 2H), 8.29-8.24 (m, 1H), 8.09-8.06 (m, 4H), 7.90-7.86 (m, 2H), 7.51-7.41 (m, 5H), 6.81-6.42 (m, 3H), 3.85 (s, 3H), 2.41 (s, 3H).

Step 3. To a solution of methyl 4-(5-(2-(2-aminopyridin-3-yl)-5-phenyl-3H-imidazo[4,5-b]pyridin-3-yl)picolinamido)-3-methylbenzoate (150 mg, 0.270 mmol) in THF (3 mL) and water (3 mL) was added LiOH·H₂O (11 mg, 0.270 mmol). The mixture was stirred at 25° C. for 2 hrs then concentrated directly. The residue was purified by prep-HPLC (Column: Welch Xtimate C18 150*30 mm*5 μm; mobile phase: [water (FA)-ACN]; B %: 0%-35%; 25 min) to give 4-(5-(2-(2-aminopyridin-3-yl)-5-phenyl-3H-imidazo[4,5-b]pyridin-3-yl)picolinamido)-3-methylbenzoic acid 178 (17.1 mg, 12% yield) as a light yellow solid. ¹H NMR (DMSO-d₆, 400 MHz) δ_{H}=10.45 (s, 1H), 8.96-8.87 (m, 1H), 8.40-8.23 (m, 3H), 8.10-8.00 (m, 5H), 7.90-7.80 (m, 2H), 7.52-7.38 (m, 4H), 6.68 (s, 2H), 6.56 (dd, J=4.8, 7.6 Hz, 1H), 2.39 (s, 3H). HPLC Rₜ=2.914 min in 8 min chromatography, purity 100%. LCMS Rₜ=1.283 min in 2 min chromatography, purity 99.4%, MS ESI calcd, for 541.19, [M+H]⁺ 542.19, found 542.2.

### Example 148. Synthesis of 4-(5-(2-(2-aminopyridin-3-yl)-5-phenyl-3H-imidazo[4,5-b]pyridin-3-yl)picolinamido)-3-fluorobenzoic acid (Compound 179)

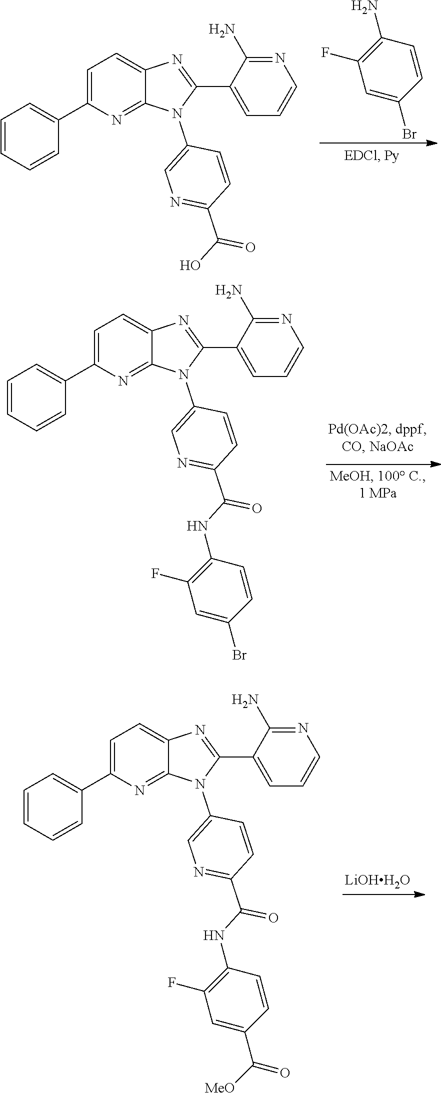

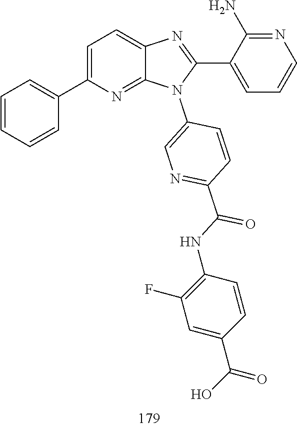 [SMILES: Nc1ncccc1-c1nc2ccc(-c3ccccc3)nc2n1-c1ccc(C(=O)Nc2ccc(C(=O)O)cc2F)nc1]

Step 1. To a mixture of 4-bromo-2-fluoroaniline (335 mg, 1.76 mmol) and intermediate 5-(2-(2-aminopyridin-3-yl)-5-phenyl-3H-imidazo[4,5-b]pyridin-3-yl) picolinic acid (600 mg, 1.47 mmol) in Pyridine (6 mL) was added EDCI (845 mg, 4.41 mmol). The mixture was stirred at 25° C. for 2 hrs. then water (40 mL) was added. The resulting mixture was extracted with EtOAc (3×60 mL) and the combined organic phase was dried over anhydrous Na₂SO₄, filtered and concentrated. The residue was purified by combi-flash column (MeOH in DCM=0%~8%) to give 5-(2-(2-aminopyridin-3-yl)-5-phenyl-3H-imidazo[4,5-b]pyridin-3-yl)-N-(4-bromo-2-fluorophenyl)picolinamide (600 mg, 65% yield) as a yellow solid. JH NMR (400 MHz, DMSO-d₆) δ=10.54 (s, 1H), 8.93 (d, J=2.0 Hz, 1H), 8.33 (d, J=7.6 Hz, 2H), 8.24 (dd, J=2.0, 8.4 Hz, 1H), 8.14-8.03 (m, 4H), 7.92 (t, J=8.4 Hz, 1H), 7.70 (dd, J=2.0, 10.4 Hz, 1H), 7.53-7.39 (m, 5H), 6.69 (s, 2H), 6.56 (dd, J=4.8, 7.6 Hz, 1H).

Step 2. To a stirred solution of 5-(2-(2-aminopyridin-3-yl)-5-phenyl-3H-imidazo[4,5-b]pyridin-3-yl)-N-(4-bromo-2-fluorophenyl)picolinamide (600 mg, 1.03 mmol) in Methanol (10 mL) and DMSO (6 mL) was added 1,1′-bis-(diphenylphosphino) ferrocene (57.0 mg, 0.103 mmol), sodium acetate (263 mg, 3.20 mmol), Pd(OAc)₂ (23.0 mg, 0.103 mmol) at 25° C. The reaction mixture was degassed and purged with N₂ for 3 times then heated to 100° C. under CO gas atmosphere (2 Mpa) and stirred for 48 hrs. The reaction mixture was concentrated and water (30 mL) was added to the residue. The resulting mixture was extracted with EtOAc (3×60 mL). The combined organic phase was washed with brine (20 mL), dried over anhydrous Na₂SO₄, filtered and concentrated. The residue was purified by flash silica gel chromatography (eluent of 0~55% EtOAc in PE) and then purified by prep-HPLC (Column: Welch Xtimate C18 40*200 mm 7 μm; mobile phase: water (NH₄HCO₃)-ACN; B %: 46%-86%; 25 min) to give methyl 4-(5-(2-(2-aminopyridin-3-yl)-5-phenyl-3H-imidazo[4,5-b]pyridin-3-yl)picolinamido)-3-fluorobenzoate (250 mg, 43% yield) as a yellow solid. ¹H NMR (400 MHz, DMSO-d₆) δ=10.63 (s, 1H), 8.95 (d, J=2.0 Hz, 1H), 8.41-8.24 (m, 4H), 8.11-8.04 (m, 4H), 7.92-7.79 (m, 2H), 7.51-7.40 (m, 4H), 6.68 (s, 2H), 6.56 (dd, J=5.2, 7.6 Hz, 1H), 3.88 (s, 3H).

Step 3. To a solution of methyl 4-(5-(2-(2-aminopyridin-3-yl)-5-phenyl-3H-imidazo[4,5-b]pyridin-3-yl)picolinamido)-3-fluorobenzoate (200 mg, 0.357 mmol) in THF (4 mL) and water (2 mL) was added LiOH·H₂O (45.0 mg, 1.07 mmol) and 2 drop of MeOH. The mixture was stirred at 40° C. for 4 hrs. The mixture was filtered. The residue was purified by prep-HPLC (Column: Welch Xtimate C18 40*200 mm 7 μm; mobile phase: water (NH₄HCO₃)-ACN; B %: 8%-48%; 25 min) to give 4-(5-(2-(2-aminopyridin-3-yl)-5-phenyl-3H-imidazo[4,5-b]pyridin-3-yl)picolinamido)-3-fluorobenzoic acid 179 (73.5 mg, 38% yield) as a light yellow solid. ¹H NMR (DMSO-d₆, 400 MHz) δ_{H}=10.60 (s, 1H), 8.95 (d, J=1.6 Hz, 1H), 8.39-8.31 (m, 2H), 8.28-8.22 (m, 2H), 8.12-8.04 (m, 4H), 7.89-7.73 (m, 2H), 7.53-7.41 (m, 4H), 6.69 (s, 2H), 6.56 (dd, J=5.2, 7.6 Hz, 1H). HPLC Rₜ=4.624 min in 8 min chromatography, purity 99.3%. LCMS Rₜ=2.632 min in 4 min chromatography, purity 99.8%, MS ESI calcd. for 545.16, [M+H]⁺ 546.16, found 546.2.

### Example 149. Synthesis of 4-(4-(2-(2-aminopyridin-3-yl)-5-phenyl-3H-imidazo[4,5-b]pyridin-3-yl)benzamido)-3-fluorobenzoic acid (Compound 180)

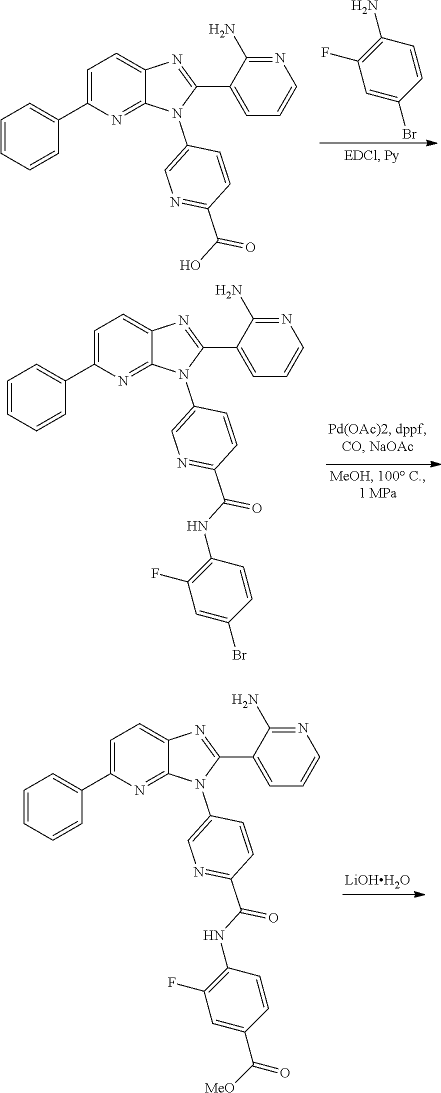

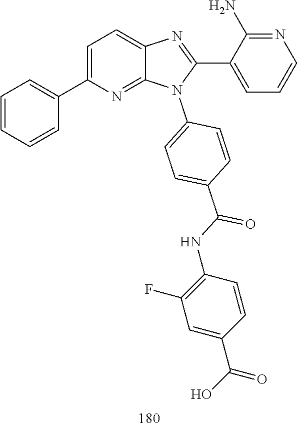 [SMILES: Nc1ncccc1-c1nc2ccc(-c3ccccc3)nc2n1-c1ccc(C(=O)Nc2ccc(C(=O)O)cc2F)cc1]

Step 1. To a solution of 4-(2-(2-aminopyridin-3-yl)-5-phenyl-3H-imidazo[4,5-b]pyridin-3-yl)benzoic acid (200 mg, 0.491 mmol) and 4-bromo-2-fluoro-aniline (93.0 mg, 0.491 mmol) in Pyridine (10 mL) was added EDCI (141 mg, 0.736 mmol). The mixture was stirred at 25° C. for 12 hrs. Water (5 mL) was added to the reaction mixture. The resulting mixture was extracted with EtOAc (3×10 mL). The combined organic phase was washed with brine (10 mL), dried over anhydrous Na₂SO₄, filtered and concentrated. The residue was purified by flash silica gel chromatography (eluent of 0~12% MeOH in DCM) to give 4-(2-(2-aminopyridin-3-yl)-5-phenyl-3H-imidazo[4,5-b]pyridin-3-yl)-N-(4-bromo-2-fluorophenyl)benzamide (240 mg, 84% yield) as a light yellow solid. ¹H NMR (DMSO-d₆, 400 MHz) δ_{H}=10.38 (s, 1H), 8.30 (d, J=8.4 Hz, 1H), 8.13 (d, J=8.4 Hz, 2H), 8.09-8.01 (m, 4H), 7.71-7.58 (m, 4H), 7.50-7.38 (m, 4H), 7.31-7.24 (m, 1H), 6.90 (s, 2H), 6.48 (dd, J=4.8, 7.6 Hz, 1H).

Step 2. To a solution of 4-(2-(2-aminopyridin-3-yl)-5-phenyl-3H-imidazo[4,5-b]pyridin-3-yl)-N-(4-bromo-2-fluorophenyl)benzamide (240 mg, 0.414 mmol) and sodium acetate (102 mg, 1.24 mmol) in Methanol (5 mL) and DMF (1 mL) was added dppf (23.0 mg, 0.0414 mmol) and Pd(OAc)₂ (14.0 mg, 0.0621 mmol) at RT. The reaction mixture was degassed and purged with CO for 3 times. The mixture was stirred at 100° C. for 12 hrs under CO (2 MPa). The reaction mixture was concentrated. Water (5 mL) was added to the residue. The resulting mixture was extracted with EtOAc (3×10 mL). The combined organic phase was washed with brine (10 mL), dried over anhydrous Na₂SO₄, filtered and concentrated. The residue was purified by flash silica gel chromatography (eluent of 0~10% MeOH in DCM) to give methyl 4-(4-(2-(2-aminopyridin-3-yl)-5-phenyl-3H-imidazo[4,5-b]pyridin-3-yl)benzamido)-3-fluorobenzoate (320 mg, 83% yield) as yellow oil. LCMS Rₜ=0.607 min in 1 min chromatography, purity 100%, MS ESI calcd, for 558.18, [M+H]⁺ 559.18, found 559.2.

Step 3. To a solution of methyl 4-(4-(2-(2-aminopyridin-3-yl)-5-phenyl-3H-imidazo[4,5-b]pyridin-3-yl)benzamido)-3-fluorobenzoate (320 mg, 0.573 mmol) in THF (4 mL) and Water (4 mL) was added LiOH·H₂O) (48.0 mg, 1.15 mmol). The mixture was stirred at 25° C. for 2 hrs. The reaction mixture was concentrated directly. The residue was purified by prep-HPLC (Column: Welch Xtimate C18 150*30 mm*5 μm; mobile phase: [water (NaHCO₃)-ACN]; B %: 0%-35%; 25 min) to give 4-(4-(2-(2-aminopyridin-3-yl)-5-phenyl-3H-imidazo[4,5-b]pyridin-3-yl)benzamido)-3-fluorobenzoic acid 180 (38.9 mg, 13% yield) as a light yellow solid. ¹H NMR (DMSO-d₆, 400 MHz) δ_{H}=10.52 (s, 1H), 8.30 (d, J=8.4 Hz, 1H), 8.14 (d, J=8.4 Hz, 2H), 8.09-8.01 (m, 4H), 7.90-7.73 (m, 3H), 7.70 (d, J=8.4 Hz, 2H), 7.52-7.37 (m, 3H), 7.29 (dd, J=1.6, 7.6 Hz, 1H), 6.90 (s, 2H), 6.49 (dd, J=4.8, 7.6 Hz, 1H). HPLC Rₜ=2.709 min in 8 min chromatography, purity 99.9%. LCMS Rₜ=1.226 min in 2 min chromatography, purity 99.8%, MS ESI calcd, for 544.17, [M+H]⁺ 545.17, found 545.2.

### Example 150. Synthesis of 4-((6-(2-(2-aminopyridin-3-yl)-5-phenyl-3H-imidazo[4,5-b]pyridin-3-yl)-2-methylpyridin-3-yl)carbamoyl)-2-methylbenzoic acid (Compound 181)

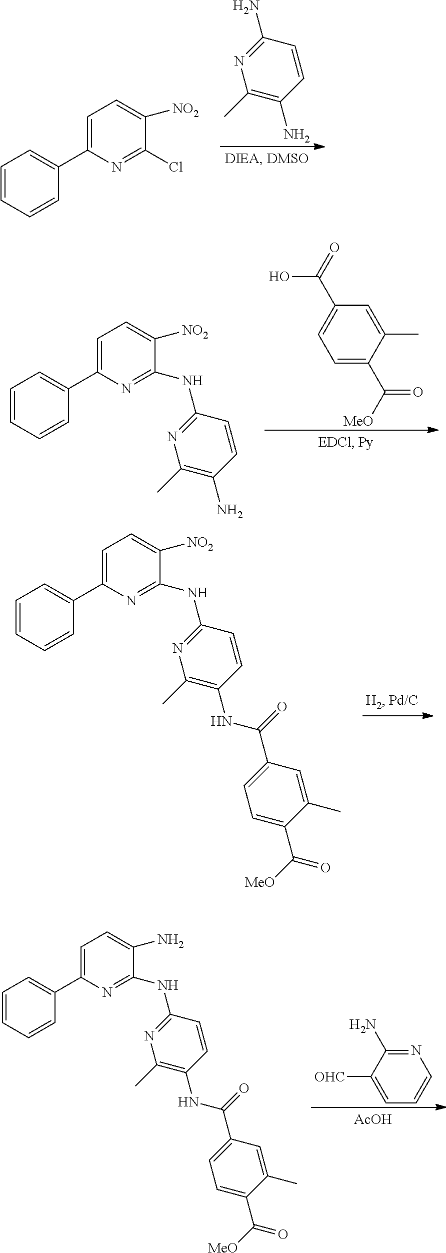

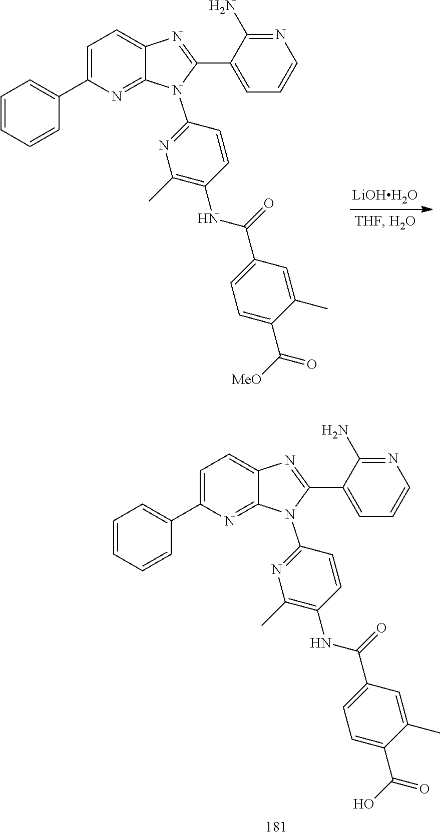

Step 1. To a solution of 2-chloro-3-nitro-6-phenylpyridine (4.00 g, 17.0 mmol) and 6-methylpyridine-2,5-diamine (2.52 g, 20.5 mmol) in DMSO (50 mL) was added DIEA (8.5 mL, 51.1 mmol). Then the mixture was stirred at 80° C. for 12 hrs. Water (10 mL) was added to the residue. The resulting mixture was extracted with EtOAc (10 mL*3). The combined organic phase was washed with brine (20 mL), dried over anhydrous Na₂SO₄, filtered and concentrated. The residue was purified by flash silica gel chromatography (eluent of 0~10% MeOH in DCM) to give 6-methyl-N₂-(3-nitro-6-phenylpyridin-2-yl)pyridine-2,5-diamine (5.38 g, 15.2 mmol) as a yellow solid. LCMS Rₜ=0.549 min in 1 min chromatography, purity 91.0%, MS ESI calcd, for 321.34, [M+H]⁺ 322.34, found 322.0.

Step 2. To a solution of 6-methyl-N₂-(3-nitro-6-phenylpyridin-2-yl)pyridine-2,5-diamine (496 mg, 1.54 mmol) in Pyridine (5 mL) was added 4-(methoxycarbonyl)-3-methylbenzoic acid (250 mg, 1.29 mmol) and EDCI (296 mg, 1.54 mmol) at 25° C. The mixture was stirred at 40° C. for 16 hrs. Water (50 mL) was added to the reaction and the reaction mixture was stirred about 2 hrs. Then the reaction mixture was filtered. The filter cake was washed with EtOAc (10 mL) and water (15 mL). Then the filter cake was concentrated to give methyl 2-methyl-4-((2-methyl-6-((3-nitro-6-phenylpyridin-2-yl)amino)pyridin-3-yl)carbamoyl)benzoate (550 mg, 16.3% yield) as a red solid, which was used in the next step directly. LCMS Rₜ=1.097 min in 1.5 min chromatography, purity 19.4%, MS ESI calcd. for 497.17. [M+H]⁺ 498.17, found 498.2.

Step 3. To a solution of methyl 2-methyl-4-((2-methyl-6-((3-nitro-6-phenylpyridin-2-yl)amino)pyridin-3-yl)carbamoyl)benzoate (550 mg, 1.11 mmol) in ethyl acetate (10 mL) was added wet Pd/C (10%, 0.150 g) under Ar atmosphere. The suspension was degassed and purged with H₂ for 3 times. The reaction mixture was stirred under H₂ (15 psi) at 25° C. for 16 hrs. The reaction mixture was filtered. The filter cake was washed with EtOAc (40 mL). The filtrate was concentrated to give methyl 4-((6-((3-amino-6-phenylpyridin-2-yl)amino)-2-methylpyridin-3-yl)carbamoyl)-2-methylbenzoate (350 mg, 67.7% yield) as a yellow solid, which was used in the next step directly. LCMS Rₜ=0.864 min in 1.5 min chromatography, purity 16.8%, MS ESI calcd, for 467.20, [M+H]⁺ 468.20, found 468.2.

Step 4. To a solution of 2-aminonicotinaldehyde (183 mg, 1.50 mmol) and methyl 4-((6-((3-amino-6-phenylpyridin-2-yl)amino)-2-methylpyridin-3-yl)carbamoyl)-2-methylbenzoate (350 mg, 0.749 mmol) in acetic acid (4 mL) at 25° C. The mixture was stirred at 80° C. for 2 hrs under O₂. The reaction mixture was concentrated directly to give methyl 4-((6-(2-(2-aminopyridin-3-yl)-5-phenyl-3H-imidazo[4,5-b]pyridin-3-yl)-2-methylpyridin-3-yl)carbamoyl)-2-methylbenzoate (500 mg, 13.6% yield) as a yellow solid, which was used in the next step directly. LCMS Rₜ=0.909 min in 1.5 min chromatography, purity 11.6%, MS ESI calcd, for 569.22, [M+H]⁺ 570.22, found 570.3.

Step 5. To a solution of methyl 4-((6-(2-(2-aminopyridin-3-yl)-5-phenyl-3H-imidazo[4,5-b]pyridin-3-yl)-2-methylpyridin-3-yl)carbamoyl)-2-methylbenzoate (0.043 mL, 0.878 mmol) in THF (3 mL) was added water (3 mL) and LiOH·H₂O (108 mg, 2.63 mmol) at 25° C. The mixture was stirred at 25° C. for 2 hr. The reaction mixture was filtered. The filter cake was washed with DMF (7 mL). The filtrate was concentrated. The residue was purified by prep-HPLC (column: Welch Xtimate C18 40*200 mm 7 μm; mobile phase: water (FA)-ACN; gradient: 14%-54% B over 25 min) to give 4-((6-(2-(2-aminopyridin-3-yl)-5-phenyl-3H-imidazo[4,5-b]pyridin-3-yl)-2-methylpyridin-3-yl)carbamoyl)-2-methylbenzoic acid 181 (9.50 mg, 1.73% yield) as a light yellow solid. ¹H NMR (DMSO-d₆, 400 MHz) δ_{H}=10.28 (s, 1H), 8.30 (d, J=8.4 Hz, 1H), 8.19 (d, J=8.4 Hz, 1H), 8.07 (d, J=7.6 Hz, 2H), 8.05-8.00 (m, 2H), 7.95-7.84 (m, 4H), 7.52-7.45 (m, 2H), 7.42 (d, J=7.2 Hz, 1H), 7.22-7.18 (m, 1H), 7.05 (s, 2H), 6.46 (dd, J=4.8, 7.6 Hz, 1H), 2.61 (s, 3H), 2.34 (s, 3H). HPLC Rₜ=2.737 min in 8 min chromatography, purity 88.96%. LCMS Rₜ=0.817 min in 1.5 min chromatography, purity 93.08%, MS ESI calcd, for 555.20, [M+H]⁺ 556.20, found 556.2.

### Example 151. Synthesis of 4-((5-(2-(2-aminopyridin-3-yl)-6-phenyl-1H-benzo[d]imidazol-1-yl)-6-methylpyridin-2-yl)carbamoyl)benzoic acid (Compound 182)

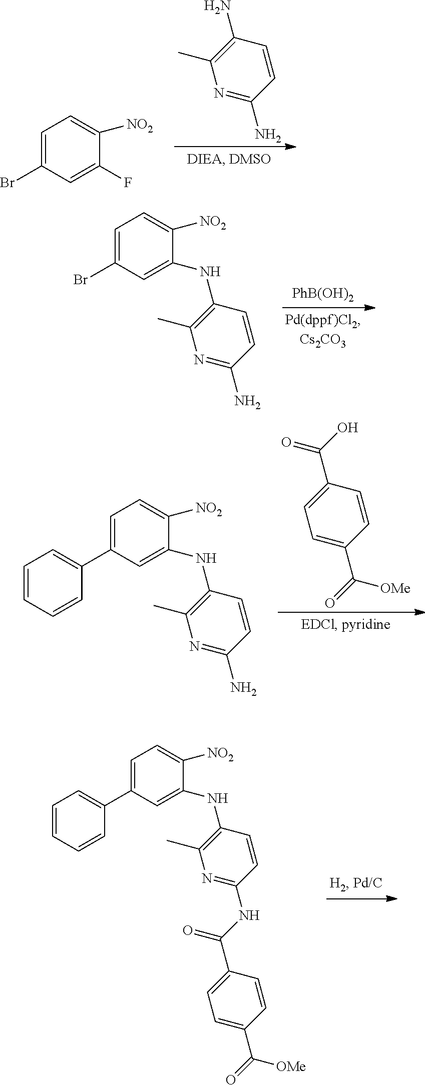

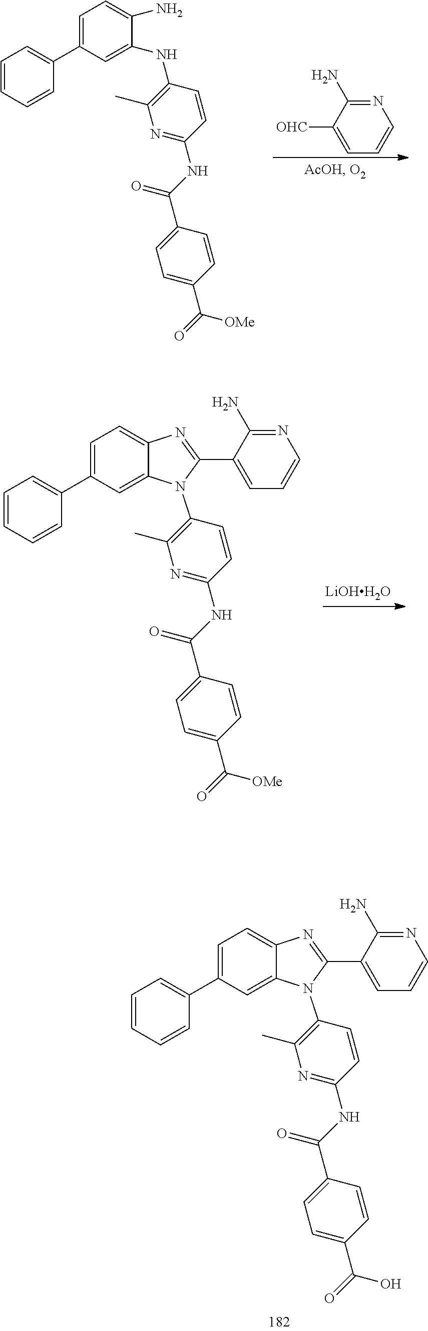

Step 1. To a solution of 4-bromo-2-fluoro-1-nitro-benzene (500 mg, 2.27 mmol) in DMSO (5 mL) was added 6-methylpyridine-2,5-diamine (280 mg, 2.27 mmol) and DIEA (881 mg, 6.82 mmol) at 25° C. The mixture was stirred at 80° C. for 12 hrs. The reaction mixture was concentrated. Water (5 mL) was added and the resulting mixture was extracted with EtOAc (10 mL*3). The combined organic phase was washed with brine (10 mL), dried over anhydrous Na₂SO₄, filtered and concentrated. The residue was purified by flash silica gel chromatography (eluent of 20~30% EtOAc in PE) to give N₅-(5-bromo-2-nitrophenyl)-6-methylpyridine-2,5-diamine (270 mg, 36.8% yield) as a yellow solid. JH NMR (DMSO-d₆, 400 MHz) δ_{H}=9.25 (s, 1H), 8.03 (d, J=9.2 Hz, 1H), 7.23 (d, J=8.4 Hz, 1H), 6.94-6.85 (m, 1H), 6.58 (d, J=2.0 Hz, 1H), 6.38 (d, J=8.4 Hz, 1H), 6.08 (s, 2H), 2.11 (s, 3H).

Step 2. To a solution of N₅-(5-bromo-2-nitrophenyl)-6-methylpyridine-2,5-diamine (270 mg, 0.836 mmol) in 1,4-Dioxane (10 mL) and water (2 mL) was added phenylboronic acid (204 mg, 1.67 mmol), Cs₂CO₃ (817 mg, 2.51 mmol) and Pd(dppf)Cl₂ (61.0 mg, 0.0836 mmol) at 25° C. The mixture was stirred at 90° C. for 12 hrs. Water (15 mL) was added to the residue. The resulting mixture was extracted with EtOAc (20 mL*3). The combined organic phase was washed with brine (20 mL), dried over anhydrous Na₂SO₄, filtered and concentrated. The residue was purified by flash silica gel chromatography (eluent of 35~40% EtOAc in PE) to give 6-methyl-N₅-(4-nitro-[1,1′-biphenyl]-3-yl)pyridine-2,5-diamine (170 mg, 63.5% yield) as a red solid. ¹H NMR (DMSO-d₆, 400 MHz) δ_{H}=9.28 (s, 1H), 8.20 (d, J=8.8 Hz, 1H), 7.49-7.41 (m, 5H), 7.30 (d, J=8.4 Hz, 1H), 7.04 (dd, J=1.6, 8.8 Hz, 1H), 6.66 (d, J=1.6 Hz, 1H), 6.38 (d, J=8.4 Hz, 1H), 6.02 (s, 2H), 2.16 (s, 3H).

Step 3. To a solution of 6-methyl-N₅-(4-nitro-[1, 1′-biphenyl]-3-yl)pyridine-2,5-diamine (500 mg, 1.56 mmol) and 4-(methoxycarbonyl)benzoic acid (337 mg, 1.87 mmol) in Pyridine (10 mL) was added EDCI (448 mg, 2.34 mmol). The mixture was stirred at 80° C. for 12 hrs. Water (10 mL) was added and the resulting mixture was extracted with EtOAc (20 mL*3). The combined organic phase was washed with brine (10 mL), dried over anhydrous Na₂SO₄, filtered and concentrated. The residue was purified by flash silica gel chromatography (eluent of 20~30% EtOAc in PE) to give methyl 4-((6-methyl-5-((4-nitro-[1,1′-biphenyl]-3-yl)amino)pyridin-2-yl)carbamoyl)benzoate (500 mg, 56.4% yield) as a yellow solid. ¹H NMR (DMSO-d₆, 400 MHz) δ_{H}=11.09 (s, 1H), 9.48 (s, 1H), 8.25 (d, J=8.8 Hz, 1H), 8.18-8.13 (m, 3H), 8.09-8.05 (m, 2H), 7.87 (d, J=8.8 Hz, 1H), 7.55-7.51 (m, 2H), 7.48-7.45 (m, 2H), 7.44-7.43 (m, 1H), 7.14 (dd, J=2.0, 8.8 Hz, 1H), 6.80 (d, J=2.0 Hz, 1H), 3.90 (s, 3H), 2.41 (s, 3H).

Step 4. To a solution of methyl 4-((6-methyl-5-((4-nitro-[1, 1′-biphenyl]-3-yl)amino)pyridin-2-yl)carbamoyl)benzoate (500 mg, 1.04 mmol) in THF (10 mL) was added Pd/C (200 mg, purity 10%) under N₂. The reaction mixture was degassed and purged with H₂ (30 psi) for 3 times. The mixture was stirred at 25° C. for 12 hrs under H₂ (30 psi). The reaction mixture was filtered and the filtrate was concentrated directly to give methyl 4-((5-((4-amino-[1,1′-biphenyl]-3-yl)amino)-6-methylpyridin-2-yl)carbamoyl)benzoate (120 mg, 25.6% yield) as light yellow oil, which was used in the next step directly.

Step 5. To a solution of methyl 4-((5-((4-amino-[1,1′-biphenyl]-3-yl)amino)-6-methylpyridin-2-yl)carbamoyl)benzoate (100 mg, 0.221 mmol) in acetic acid (3 mL) was added 2-aminonicotinaldehyde (32 mg, 0.265 mmol) under 02. The mixture was stirred at 80° C. for 1 hr under 02. The reaction mixture was concentrated directly to give methyl 4- ((5-(2-(2-aminopyridin-3-yl)-6-phenyl-1H-benzo[d]imidazol-1-yl)-6-methylpyridin-2-yl)carbamoyl)benzoate (100 mg, 28.6% yield) as light yellow oil. LCMS Rₜ=0.638 min in 1 min chromatography, purity 40.99%, MS ESI calcd, for 554.21 [M+H]⁺ 555.21, found 555.3.

Step 6. To a solution of methyl 4-((5-(2-(2-aminopyridin-3-yl)-6-phenyl-1H-benzo[d]imidazol-1-yl)-6-methylpyridin-2-yl)carbamoyl)benzoate (138 mg, 0.249 mmol) in THF (3 mL) and water (3 mL) was added LiOH·H₂O (21 mg, 0.498 mmol) at 25° C. The mixture was stirred at 25° C. for 12 hrs. The reaction mixture was concentrated directly. The residue was purified by prep-HPLC (Column: Welch Xtimate C18 150*30 mm*5 μm; mobile phase: [water (NH₄HCO₃)-ACN]; B %: 8%-48%; 25 min) to give 4-((5-(2-(2-aminopyridin-3-yl)-6-phenyl-1H-benzo[d]imidazol-1-yl)-6-methylpyridin-2-yl)carbamoyl)benzoic acid 182 (14.2 mg, 10.5% yield) as a light yellow solid. ¹H NMR (DMSO-d₆, 400 MHz) δ_{H}=11.24 (s, 1H), 8.31 (d, J=8.8 Hz, 1H), 8.15-8.07 (m, 3H), 8.06-7.99 (m, 3H), 7.92 (d, J=8.4 Hz, 1H), 7.73-7.64 (m, 3H), 7.47-7.40 (m, 2H), 7.36-7.29 (m, 2H), 7.24-7.14 (m, 3H), 6.45 (dd, J=4.8, 7.6 Hz, 1H), 1.98 (s, 3H). HPLC R; =2.878 min in 8 min chromatography, purity 99.52%. LCMS Rₜ=1.309 min in 2 min chromatography, purity 99.21%, MS ESI calcd. for 540.58. [M+H]⁺ 541.58, found 541, 1.

### Example 152. Synthesis of 4-((4-(4-(2-(2-aminopyridin-3-yl)-5-phenyl-3H-imidazo[4,5-b]pyridin-3-yl)phenyl)piperidin-1-yl)methyl)benzoic acid (Compound 139)

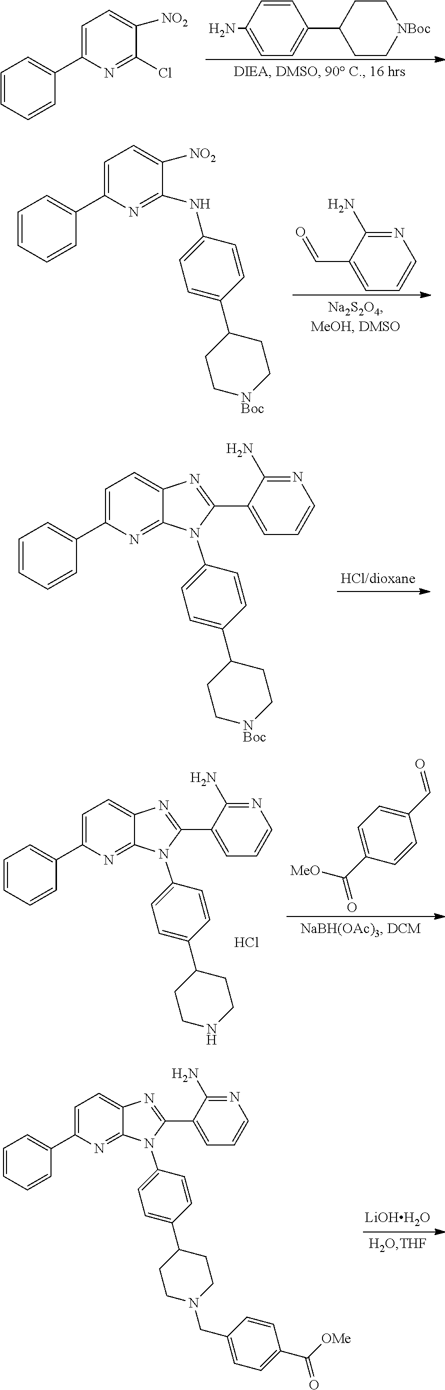

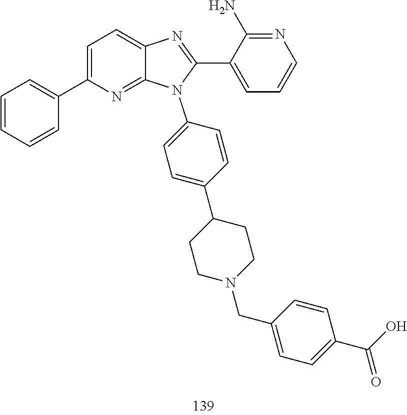 [SMILES: Nc1ncccc1-c1nc2ccc(-c3ccccc3)nc2n1-c1ccc(C2CCN(Cc3ccc(C(=O)O)cc3)CC2)cc1]

Step 1. To a solution of tert-butyl 4-(4-aminophenyl)piperidine-1-carboxylate (2.83 g, 10.2 mmol) in DMSO (20 mL) was added DIEA (3.30 g, 25.6 mmol) and 2-chloro-3-nitro-6-phenyl-pyridine (2.00 g, 8.52 mmol) at 25° C., and the mixture was stirred at 80° C. for 14 hrs. EA (50 mL) was added to the reaction, and the resulting suspension was washed with brine (40 mL×3), dried over anhydrous Na₂SO₄, filtered and concentrated. The mixture was triturated with MeCN (50 mL) and filtered. The filter cake was concentrated to give tert-butyl 4-[4-[(3-nitro-6-phenyl-2-pyridyl)amino]phenyl]piperidine-1-carboxylate (3.30 g, 6.95 mmol, 81.6% yield) as a red solid. LCMS Rₜ=1.877 min in 2 min chromatography, purity 62.7%, MS ESI calcd, for 474.23, [M+H]⁺ 475.23, found 475.3.

Step 2. To a solution of tert-butyl 4-[4-[(3-nitro-6-phenyl-2-pyridyl)amino]phenyl]piperidine-1-carboxylate (2.80 g, 5.90 mmol) and 2-aminopyridine-3-carbaldehyde (1.44 g, 11.8 mmol) in DMSO (28 mL) and methanol (2 mL) was added Na₂S₂O₄ (5.96 g, 34.2 mmol) under N₂. The mixture was stirred at 100° C. for 12 hrs. The reaction mixture was concentrated. Water (40 mL) was added to the residue. The resulting mixture was extracted with EtOAc (60 mL×3). The combined organic phase was washed with brine (50 mL), dried over anhydrous Na₂SO₄, filtered and concentrated. The residue was purified by flash silica gel chromatography (0-52% of EtOAc in PE) to give tert-butyl 4-[4-[2-(2-amino-3-pyridyl)-5-phenyl-imidazo[4,5-b]pyridin-3-yl]phenyl]piperidine-1-carboxylate (1.23 g, 2.25 mmol, 38% yield) as a yellow solid. LCMS Rₜ=0.886 min in 1.5 min chromatography, purity 25.7%, MS ESI calcd, for 546.27, [M+H]⁺ 547.27, found 547.7.

Step 3. To a solution of tert-butyl 4-[4-[2-(2-amino-3-pyridyl)-5-phenyl-imidazo[4,5-b]pyridin-3-yl]phenyl]piperidine-1-carboxylate (0.400 g, 0.732 mmol) in 1,4-dioxane (2 mL) was added HCl/dioxane (26 mL, 52.2 mmol). The mixture was stirred at 25° C. for 12 hrs. The reaction mixture was concentrated to give 3-[5-phenyl-3-[4-(4-piperidyl)phenyl]imidazo[4,5-b]pyridin-2-yl]pyridin-2-amine (500 mg, 1.12 mmol, HCl salt) as a yellow solid. LCMS Rₜ=0.618 min in 2.0 min chromatography, purity 94.6%, MS ESI calcd, for 446.22. [M+H]⁺ 447.22, found 447.2.

Step 4. To a solution of 3-[5-phenyl-3-[4-(4-piperidyl)phenyl]imidazo[4,5-b]pyridin-2-yl]pyridin-2-amine; hydrochloride (100 mg, 0.207 mmol) in DCM (2.00 mL) was added methyl 4-formylbenzoate (42.0 mg, 0.253 mmol), TEA (0.028 mL, 0.207 mmol) under N₂ atmosphere. NaBH(OAc)₃ (132 mg, 0.621 mmol) was then added into the reaction mixture. The reaction mixture was degassed and purged with N₂ for 3 times. The mixture was stirred at 20° C. for 6 hrs. The reaction mixture was taken up in EtOAc (50 mL) and washed with water (30 mL) and brine (3× 20 mL). The organic layers were then separated and dried over Na₂SO₄ before concentration to dryness. The crude was then purified by flash column chromatography eluting with 70% of EtOAc in isohexane. The desired fractions were concentrated to dryness in vacuo to give methyl 4-[[4-[4-[2-(2-amino-3-pyridyl)-5-phenyl-imidazo[4,5-b]pyridin-3-yl]phenyl]-1-piperidyl]methyl]benzoate (80.0 mg, 0.127 mmol, 61% yield) as a light yellow solid. LCMS Rₜ=0.397 min in 1 min chromatography, purity 93.1%, MS ESI calcd, for 594.27 [M+H]⁺ 595.27, found 595.1.

Step 5. To a solution of methyl 4-[[4-[4-[2-(2-amino-3-pyridyl)-5-phenyl-imidazo[4,5-b]pyridin-3-yl]phenyl]-1-piperidyl]methyl]benzoate (80.0 mg, 0.135 mmol) in THF (2.00 mL), water (1.00 mL), methanol (0.5 mL) was added LiOH·H₂O (9.30 mg, 0.404 mmol) under N₂ atmosphere. The mixture was stirred at 40° C. for 6 hrs. The reaction mixture was concentrated and then purified by prep-HPLC (column: Welch Xtimate C18 150*30 mm*5 μm, mobile phase: [water (FA)-ACN]; B %: 0%-30%, 14 min) to give 4-[[4-[4-[2-(2-amino-3-pyridyl)-5-phenyl-imidazo[4,5-b]pyridin-3-yl]phenyl]-1-piperidyl]methyl]benzoic acid 139 (41.0 mg, 0.0694 mmol, 52% yield) as a light yellow solid. ¹H NMR (400 MHz, DMSO-d₆) δ=8.26 (d, J=8.4 Hz, 1H). 8.05-7.96 (m, 4H), 7.88 (d, J=8.0 Hz, 2H), 7.50-7.36 (m, 9H), 7.17 (dd, J=2.0, 7.6 Hz, 1H), 6.96 (s, 2H), 6.40 (dd, J=4.8, 7.6 Hz, 1H), 3.57 (s, 2H), 2.94 (d, J=11.2 Hz, 2H), 2.63-2.60 (m, 1H), 2.14-2.06 (m, 2H), 1.86-1.71 (m, 4H). HPLC Rₜ=2.260 min in 8 min chromatography, purity 98.8%. LCMS Rₜ=1.576 min in 4 min chromatography, purity 96.1%, MS ESI calcd, for 580.26 [M+H]⁺ 581.26, found 581.3.

### Example 153. Synthesis of 4-((3-(4-(2-(2-aminopyridin-3-yl)-5-phenyl-3H-imidazo[4,5-b]pyridin-3-yl)phenyl) azetidin-1-yl)methyl)benzoic acid (Compound 183)

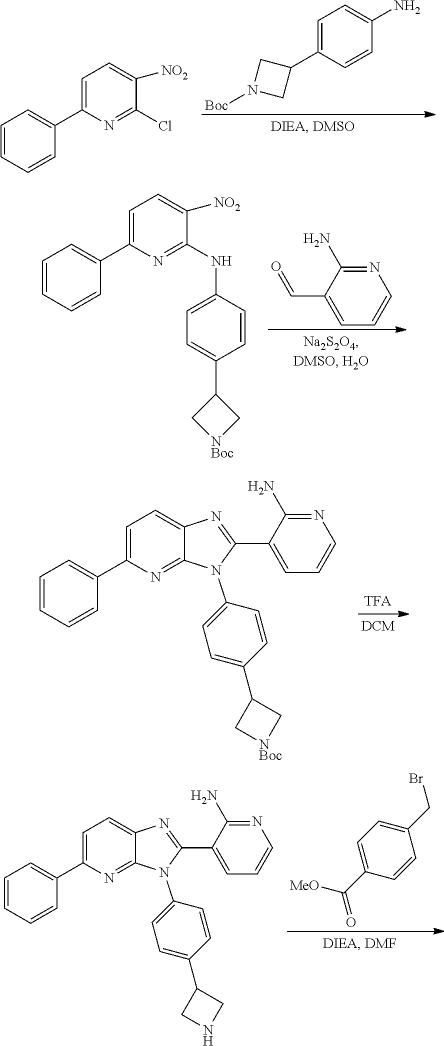

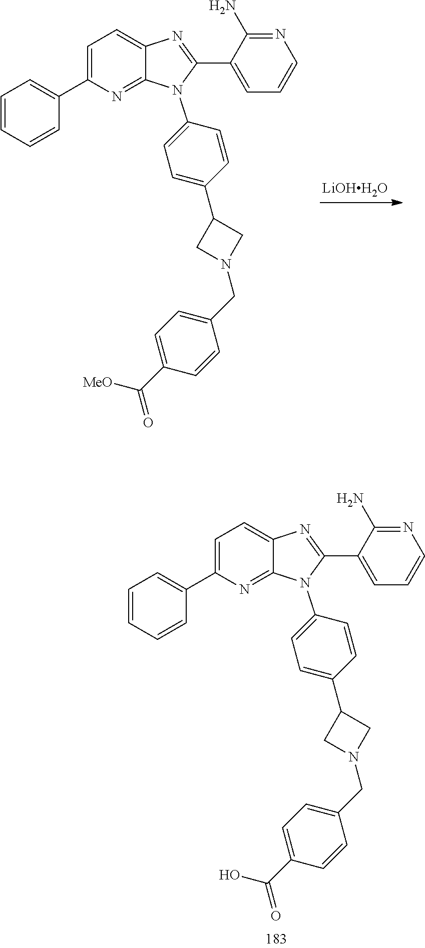 [SMILES: COC(=O)c1ccc(CN2CC(c3ccc(-n4c(-c5cccnc5N)nc5ccc(-c6ccccc6)nc54)cc3)C2)cc1 ; "LiOH·H2O" ; Nc1ncccc1-c1nc2ccc(-c3ccccc3)nc2n1-c1ccc(C2CN(Cc3ccc(C(=O)O)cc3)C2)cc1]

Step 1. To a solution of 2-chloro-3-nitro-6-phenylpyridine (2.00 g, 8.05 mmol) in DMSO (20.0 mL) was added tert-butyl 3-(4-aminophenyl) azetidine-1-carboxylate (2.00 g, 8.05 mmol) and DIEA (2.10 mL, 12.3 mmol) at 25° C. The mixture was stirred at 80° C. for 16 hrs. Water (200 mL) was added. The resulting mixture was extracted with EtOAc (200 mL×3). The combined organic phase was washed with brine (50 mL), water (50 mL), dried over anhydrous Na₂SO₄, filtered and concentrated. The residue was purified by flash silica gel chromatography (eluent of 0-33% EtOAc/PE @, 45 mL/min) to give tert-butyl 3-(4-((3-nitro-6-phenylpyridin-2-yl)amino)phenyl) azetidine-1-carboxylate (1.20 g, 67% yield) as a red solid. ¹H NMR (400 MHz, CHLOROFORM-d) δ=10.31 (s, 1H), 8.59 (d, J=8.8 Hz, 1H), 8.10-8.00 (m, 2H), 7.76 (d, J=8.4 Hz, 2H), 7.57-7.45 (m, 3H), 7.38 (d, J=8.4 Hz, 2H), 7.31 (d, J=8.8 Hz, 1H), 4.36 (t, J=8.8 Hz, 2H), 4.02 (dd, J=8.4, 6.0 Hz, 2H), 3.82-3.73 (m, 1H), 1.49 (s, 9H).

Step 2. To a solution of tert-butyl 3-(4-((3-nitro-6-phenylpyridin-2-yl)amino)phenyl) azetidine-1-carboxylate (2.30 g, 5.15 mmol) in DMSO (23.0 mL) and methanol (23.0 mL) was added 2-aminonicotinaldehyde (849 mg, 6.95 mmol) and Na₂S₂O₄ (1.79 g, 10.3 mmol) at 25° C. The mixture was stirred at 100° C. for 16 hrs. The reaction mixture was concentrated to remove MeOH. Water (50 mL) was added to the residue. The resulting mixture was extracted with EtOAc (100 mL×3). The combined organic phase was washed with brine (20 mL), water (20 mL), dried over anhydrous Na₂SO₄, filtered and concentrated. The residue was purified by flash silica gel chromatography (eluent of 0-66% EtOAc in PE) to give tert-butyl 3-(4-(2-(2-aminopyridin-3-yl)-5-phenyl-3H-imidazo[4,5-b]pyridin-3-yl)phenyl) azetidine-1-carboxylate (400 mg, 17% yield) as a yellow oil. ¹H NMR (400 MHz, DMSO-d₆) δ=8.74 (d, J=2.4 Hz, 1H), 8.71 (d, J=2.4 Hz, 1H), 8.34-8.28 (m, 3H), 8.08 (dd, J=2.0, 8.4 Hz, 2H), 7.94 (dd, J=2.0, 4.8 Hz, 1H), 7.51 (d, J=8.4 Hz, 2H), 7.39 (dd, J=6.4, 8.4 Hz, 4H), 7.13 (dd, J=1.6, 7.6 Hz, 1H), 6.49-6.43 (m, 1H), 3.17 (s, 2H), 3.16 (s, 2H), 3.05 (s, 1H), 1.40 (s, 9H).

Step 3. To a solution of tert-butyl 3-(4-(2-(2-aminopyridin-3-yl)-5-phenyl-3H-imidazo[4,5-b]pyridin-3-yl)phenyl) azetidine-1-carboxylate (200 mg, 0.386 mmol) in DCM (4.00 mL) was added TFA (2.00 mL) at 25° C. The mixture was stirred at 25° C. for 2 hrs. The reaction mixture was concentrated to give 3-(3-(4-(azetidin-3-yl)phenyl)-5-phenyl-3H-imidazo[4,5-b]pyridin-2-yl)pyridin-2-amine (161 mg, 99.8% yield, TFA salt) as a brown oil, which was used in the next step directly. LCMS Rₜ=0.410 min in 1 min chromatography, purity 79.5%, MS ESI calcd, for 418.19 [M+H]⁺ 419.19, found 419.1.

Step 4. To a stirred solution of 3-[3-[4-(azetidin-3-yl)phenyl]-5-phenyl-imidazo[4,5-b]pyridin-2-yl]pyridin-2-amine (160 mg, 0.380 mmol) in DMF (2 mL) was added DIEA (247 mg, 1.91 mmol). Methyl 4-(bromomethyl)benzoate (88.0 mg, 0.382 mmol) was then added to the reaction mixture. The reaction mixture was stirred at 25° C. under N₂ for 10 hrs. The reaction mixture was diluted with EtOAc (20 mL) and washed with water (15 mL) and brine (15 mL×2), dried over anhydrous sodium sulfate and filtered. The filtrate was concentrated to afford methyl 4-[[3-[4-[2-(2-amino-3-pyridyl)-5-phenyl-imidazo[4,5-b]pyridin-3-yl]phenyl]azetidin-1-yl]methyl]benzoate (100 mg, 46.2% yield) as a yellow solid. LCMS Rₜ=0.40 min in 1.0 min chromatography. purity 68.6%, MS ESI calcd, for 566.24 [M+H]⁺ 567.24, found 567.3.

Step 5. To a stirred solution of methyl 4-[[3-[4-[2-(2-amino-3-pyridyl)-5-phenyl-imidazo[4,5-b]pyridin-3-yl]phenyl]azetidin-1-yl]methyl]benzoate (100 mg, 0.170 mmol) in THF (1 mL) and water (1 mL) was added LiOH·H₂O (14.0 mg, 0.350 mmol). The reaction mixture was stirred at 25° C. under N₂ for 2 hrs. The reaction mixture was adjusted to around 5 with 1N HCl solution and concentrated to afford a residue. The crude product was purified by prep-HPLC (column: welch xtimate C18 150×30 mm×Sum, method: water (FA)-CAN, begin B: 17, end B 47) to afford 4-[3-[4-[2-(2-amino-3-pyridyl)-5-phenyl-imidazo[4,5-b]pyridin-3-yl]phenyl]azetidin-1-yl]methyl]benzoic acid 183 (4.90 mg, 5% yield) as a white solid. ¹H NMR (400 MHz, DMSO-d₆) δ=8.25 (d, J=8.4 Hz, 1H), 8.05-7.82 (m, 6H), 7.60-7.33 (m, 9H), 7.21 (dd, J=2.0, 7.6 Hz, 1H), 6.42 (dd, J=4.8, 7.6 Hz, 1H), 3.83-3.67 (m, 5H), 3.33-3.25 (m, 2H). HPLC Rₜ=3.054 min in 8 min chromatography, purity 99.5%. LCMS Rₜ=1.636 min in 4 min chromatography, purity 97.9%, MS ESI calcd, for 552.23 [M+H]⁺ 553.23, found 553.3.

### Example 154. Synthesis of 4-((3-(4-(2-(2-aminopyridin-3-yl)-5-phenyl-3H-imidazo[4,5-b]pyridin-3-yl)phenyl) azetidin-1-yl)methyl)-2-methoxybenzoic acid (Compound 184)

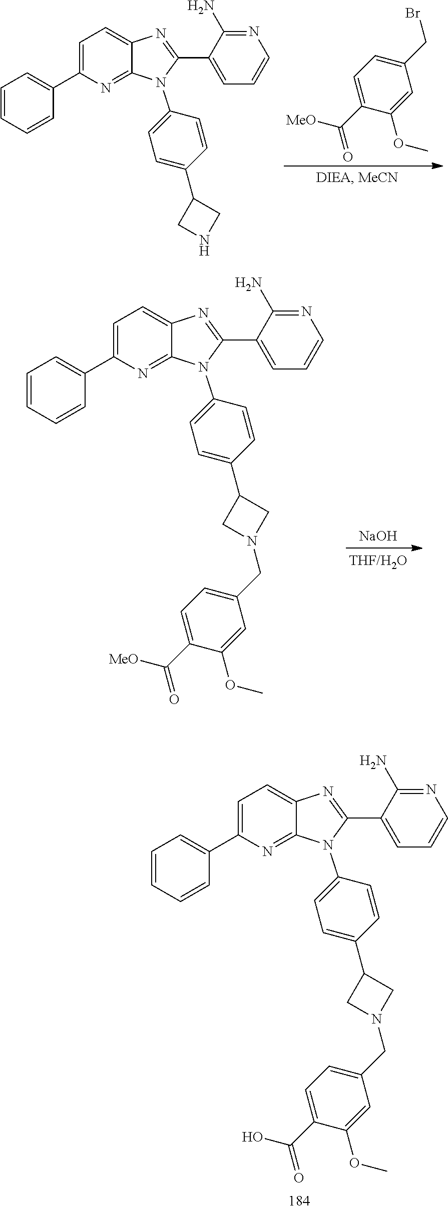 [SMILES: Nc1ncccc1-c1nc2ccc(-c3ccccc3)nc2n1-c1ccc(C2CNC2)cc1 ; COC(=O)c1ccc(CBr)cc1OC ; COC(=O)c1ccc(CN2CC(c3ccc(-n4c(-c5cccnc5N)nc5ccc(-c6ccccc6)nc54)cc3)C2)cc1OC ; "NaOH" ; COc1cc(CN2CC(c3ccc(-n4c(-c5cccnc5N)nc5ccc(-c6ccccc6)nc54)cc3)C2)ccc1C(=O)O]

Step 1. To the solution of 3-[3-[4-(azetidin-3-yl)phenyl]-5-phenyl-imidazo[4,5-b]pyridin-2-yl]pyridin-2-amine (50.0 mg, 0.119 mmol) in MeCN (2.00 mL) was added methyl 4-(bromomethyl)-2-methoxy-benzoate (43.0 mg, 0.167 mmol) and DIEA (0.110 mL, 0.597 mmol). The mixture was stirred at 30° C. for 16 hrs. H₂O (40 mL) was added and the mixture extracted with DCM (30 mL×3), The combined organic layer was dried over Na₂SO₄, filtered and concentrated in vacuum. The crude was purified by flash column (0-30% of MeOH in DCM) to give methyl 4-[[3-[4-[2-(2-amino-3-pyridyl)-5-phenyl-imidazo[4,5-b]pyridin-3-yl]phenyl]azetidin-1-yl]methyl]-2-methoxy-benzoate (50.0 mg, 70% yield) as a yellow solid. ¹H NMR (400 MHz, DMSO-d₆) δ=8.26 (d, J=8.4 Hz, 1H), 8.04-7.97 (m, 4H), 7.61 (d, J=7.6 Hz, 1H), 7.59-7.52 (m, 2H), 7.52-7.43 (m, 4H), 7.43-7.37 (m, 1H), 7.21 (dd, J=1.6, 7.6 Hz, 1H), 7.10 (s, 1H), 7.02-6.89 (m, 3H), 6.42 (dd, J=4.8, 7.6 Hz, 1H), 3.83 (s, 3H), 3.77 (s. 3H), 3.75-3.66 (m, 5H), 3.25 (s, 2H).

Step 2. To a solution of methyl 4-[[3-[4-[2-(2-amino-3-pyridyl)-5-phenyl-imidazo[4,5-b]pyridin-3-yl]phenyl]azetidin-1-yl]methyl]-2-methoxy-benzoate (50.0 mg, 0.0838 mmol) in water (1 mL) and THF (1 mL) was added NaOH (10.0 mg, 0.251 mmol). The reaction mixture was stirred at 30° C. for 16 hrs. The reaction mixture was concentrated and purified by prep-HPLC (column: Xtimate C18 150*40 mm*5 μm; mobile phase: water (FA)-ACN: B %: 15%-45%, 60 min) to give 4-[[3-[4-[2-(2-amino-3-pyridyl)-5-phenyl-imidazo[4,5-b]pyridin-3-yl]phenyl]azetidin-1-yl]methyl]-2-methoxy-benzoic acid 184 (16.0 mg, 31.8% yield) as a yellow solid. ¹H NMR (400 MHz, DMSO-d₆) δ=8.27 (d, J=8.4 Hz, 1H), 8.18 (s, 1H), 8.04-7.99 (m, 3H), 7.97 (s, 1H), 7.60 (d, J=7.6 Hz, 1H), 7.57-7.53 (m, 2H), 7.50-7.44 (m, 4H), 7.43-7.38 (m, 1H), 7.21 (dd, J=1.6, 7.6 Hz, 1H). 7.06 (s, 1H), 6.98-6.91 (m, 3H), 6.42 (dd, J=4.8, 7.6 Hz, 1H), 3.82 (s, 3H), 3.73-3.69 (m, 4H), 3.25-3.22 (m, 3H). HPLC Rₜ=3.663 min in 8 min chromatography, purity 99.6%. LCMS Rₜ=2.876 min in 4 min chromatography, purity 99.3%, MS ESI calcd. For 582.24 [M+H]⁺ 583.24, found 583.3.

### Example 155. Synthesis of 4-((3-(4-(2-(2-aminopyridin-3-yl)-5-phenyl-3H-imidazo[4,5-b]pyridin-3-yl)phenyl) azetidin-1-yl)methyl)-2-fluoro-6-hydroxybenzoic acid (Compound 185)

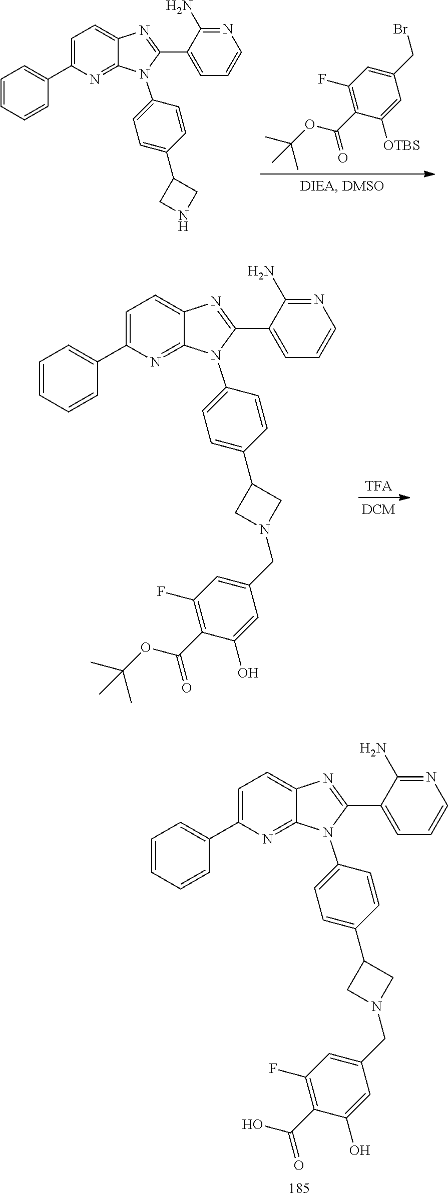 [SMILES: Nc1ncccc1-c1nc2ccc(-c3ccccc3)nc2n1-c1ccc(C2CNC2)cc1 ; CC(C)(C)OC(=O)c1c(F)cc(CBr)cc1O[Si](C)(C)C(C)(C)C ; CC(C)(C)OC(=O)c1c(O)cc(CN2CC(c3ccc(-n4c(-c5cccnc5N)nc5ccc(-c6ccccc6)nc54)cc3)C2)cc1F ; "TFA" ; Nc1ncccc1-c1nc2ccc(-c3ccccc3)nc2n1-c1ccc(C2CN(Cc3cc(O)c(C(=O)O)c(F)c3)C2)cc1]

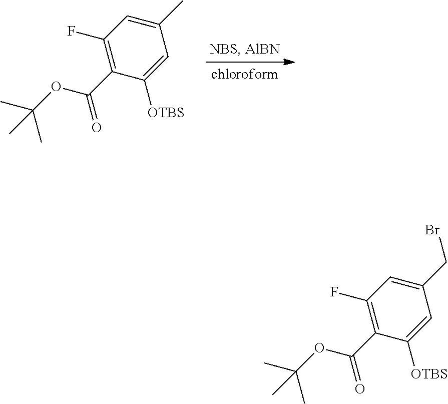 [SMILES: Cc1cc(F)c(C(=O)OC(C)(C)C)c(O[Si](C)(C)C(C)(C)C)c1 ; CC(C)(C)OC(=O)c1c(F)cc(CBr)cc1O[Si](C)(C)C(C)(C)C]

Step 1. To a solution of tert-butyl 2-[tert-butyl(dimethyl) silyl]oxy-6-fluoro-4-methyl-benzoate (100 mg, 0.294 mmol) in chloroform (6 mL) was added NBS (52.0 mg, 0.441 mmol) and AIBN (4.80 mg, 0.0294 mmol) at 25° C. under N₂. The mixture was stirred at 65° C. for 3.5 hrs. H₂O (200 mL) was added. The mixture was extracted with EtOAc (200 mL×3). The organic phase was washed with brine (200 mL), dried over Na₂SO₄, filtered and concentrated under reduced pressure. The crude was purified by flash chromatography eluting with PE/EtOAc (from 0% to 10% of EtOAc in PE) to give tert-butyl 4-(bromomethyl)-2-[tert-butyl (dimethyl) silyl]oxy-6-fluoro-benzoate (70.0 mg, 57% yield) as a yellow oil. ¹H NMR (400 MHz, CHLOROFORM-d) δ=6.74-6.69 (m, 1H), 6.63 (s, 1H), 4.35 (s, 2H), 1.57 (s, 9H), 0.99 (s, 9H), 0.26 (s, 6H).

Step 2. To a solution of 3-[3-[4-(azetidin-3-yl)phenyl]-5-phenyl-imidazo[4,5-b]pyridin-2-yl]pyridin-2-amine (40.0 mg, 0.0960 mmol) in DMSO (3 mL) was added tert-butyl 4-(bromomethyl)-2-[tert-butyl(dimethyl) silyl]oxy-6-fluoro-benzoate (44.0 mg, 0.105 mmol) and DIEA (0.300 mL, 0.0956 mmol) at 25° C. under N₂. The mixture was stirred at 25° C. for 16 hrs. H₂O (20 mL) was added. The mixture was extracted with EtOAc (20 mL×3). The organic phase was washed with brine (20 mL), dried over Na₂SO₄, filtered and concentrated under reduced pressure to give a crude. The crude was purified by flash chromatography eluting with DCM/MeOH (from 0% to 10% of MeOH in DCM) to give tert-butyl 4-[[3-[4-[2-(2-amino-3-pyridyl)-5-phenyl-imidazo[4,5-b]pyridin-3-yl]phenyl]azetidin-1-yl]methyl]-2-fluoro-6-hydroxy-benzoate (70.0 mg, impure) as a yellow solid. ¹H NMR (400 MHz, CHLOROFORM-d) δ=11.51 (br s, 1H), 8.09 (d, J=8.4 Hz, 1H), 8.06 (dd, J=1.6, 4.8 Hz, 1H), 8.01 (d, J=7.6 Hz, 2H), 7.77 (d, J=8.4 Hz, 1H), 7.47-7.39 (m, 6H), 7.38-7.31 (m, 2H), 7.11 (dd, J=1.6, 8.0 Hz, 1H), 6.74 (s, 1H), 6.68 (br s, 2H), 6.60 (d, J=12.0 Hz, 1H), 6.37 (dd, J=5.2, 8.0 Hz, 1H), 3.79 (s, 2H), 3.61 (s, 2H), 3.32-3.25 (m, 2H), 1.60 (s, 9H).

Step 3. To a solution of tert-butyl 4-[[3-[4-[2-(2-amino-3-pyridyl)-5-phenyl-imidazo[4,5-b]pyridin-3-yl]cyclohexa-1,3-dien-1-yl]azetidin-1-yl]methyl]-2-fluoro-6-hydroxy-benzoate (80.0 mg, 0.124 mmol) in DCM (2.5 mL) was added TFA (0.500 mL, 0.124 mmol) at 25° C. The mixture was stirred at 25° C. for 16 hrs. The crude was purified by prep-HPLC (Welch Xtimate C18 150*30 mm*Sum water (FA)-ACN) to give 4-[[3-[4-[2-(2-amino-3-pyridyl)-5-phenyl-imidazo[4,5-b]pyridin-3-yl]phenyl]azetidin-1-yl]methyl]-2-fluoro-6-hydroxy-benzoic acid 185 (15.6 mg, 21% yield) as a white solid. ¹H NMR (400 MHz, DMSO-d₆) δ=8.25 (d, J=8.4 Hz, 1H), 8.02-7.95 (m, 4H), 7.61-7.56 (m, 2H), 7.53-7.44 (m, 4H), 7.40 (d, J=7.6 Hz, 1H), 7.24 (dd, J=2.0, 7.6 Hz, 1H), 6.65 (s, 1H), 6.52 (d, J=10.8 Hz, 1H), 6.44 (dd, J=5.2, 8.8 Hz, 1H), 4.38-4.11 (m, 7H). HPLC Rₜ=2.488 min in 8.0 min chromatography, 10-80AB_8 min.1 cm, purity 100%. LCMS Rₜ=3.283 min in 4.0 min chromatography, purity 100%, MS ESI calcd, for 586.21 [M+H]⁺ 587.21, found 587.3.

### Example 156. AKT1 E17K Kinase Biological Activity Assay for Exemplary Compounds

AKT1 E17K kinase assay: Inhibitory effects of compounds on AKT1 E17K were undertaken in a 10-pt dose response kinase activity assay measuring phospho-peptide product formation based on TR-FRET signal as described in the following paragraph.

Purified human recombinant kinase full-length AKT1 E17K was expressed in insect cells and activated in vitro by PDPK1. Ulight™-CREBtide (PerkinElmer, catalog number TRF0107) was used as the peptide substrate. The reaction buffer consisted of 50 mM HEPES pH 7.5, 10 mM MgCl₂, 0.01% Triton X-100, 0.01% BSA, 2 mM DTT, 0.6 nM AKT1 E17K, 50 nM. Ulight™ CREBtide and either 50 μM ATP or 2 mM ATP. The compound solution was prepared as 10 mM DMSO stock. For the assay, a 1:3 serial dilution 10-pt dose response of test compounds, in duplicate, for each concentration were dispensed into a 384-well plate (Corning, catalog number 4513). Final DMSO concentration in the assay was 1%. The enzyme mixture (5 μL. 1.2 nM) were added and the mixture was incubated for 15 min at RT prior to the start of the kinase reaction by the addition of 5 μL substrate mixture (100 nM Ulight™-CREBtide). The reaction was stopped after 60 min of incubation at RT by the addition of 10 μL of Detection Mix (PerkinElmer, #CR97-100) consisting of Europium-anti-phospho-CREB (Ser133) (Perkin Elmer, catalog numberTRF0200). Incubation with the detection mix for a further 60 min allowed binding of antibody to the phospho-Ulight™ CREBtide. The fluorescence emissions at 620 nm and 665 nm after excitation at 350 om was measured using Envision reader. The ratio of the emissions at 665 nm and at 620 nm provided the measure for the amount of phosphorylated CREBtide product of AKT1 E17K kinase activity. To determine the IC₅₀ values, the data was normalized (enzyme reaction without inhibitor=0% inhibition, all other assay components but no enzyme=100% inhibition) and IC₅₀ values were calculated by a 4-parameter fit using an in-house developed protocol.

Table 4 assigns each compound a code for potency in the AKT1 E17K kinase assay: A, B, C, or D. According to the code, A represents an IC₅₀ value <50 nM; B represents an IC₅₀ value from ≥50 nM to <200 nM; C represents an IC₅₀ value from ≥200 nM to <500 nM; and D represents an IC₅₀ value ≥500 nM.

TABLE 4

| | AKT1 E17K Lance |
|---|---|
| Compound No. | IC₅₀ |
| 1 | D |
| 2 | D |
| 3 | C |
| 4 | D |
| 5 | D |
| 6 | B |
| 7 | B |
| 8 | B |
| 9 | C |
| 10 | D |
| 11 | D |
| 12 | D |
| 13 | D |
| 14 | A |
| 15 | A |
| 16 | B |
| 17 | B |
| 18 | B |
| 19 | A |
| 20 | C |
| 21 | A |
| 22 | C |
| 23 | A |
| 24 | B |
| 25 | A |
| 26 | D |
| 27 | C |
| 28 | D |
| 29 | B |
| 30 | B |
| 31 | D |
| 32 | B |
| 33 | D |
| 34 | C |
| 35 | A |
| 36 | B |
| 37 | C |
| 38 | D |
| 39 | B |
| 40 | A |
| 41 | C |
| 42 | B |
| 43 | D |
| 44 | D |
| 45 | D |
| 46 | B |
| 47 | C |
| 48 | D |
| 49 | D |
| 50 | D |
| 51 | B |
| 52 | D |
| 53 | A |
| 54 | A |
| 55 | A |
| 56 | A |
| 57 | A |
| 58 | B |
| 59 | A |
| 60 | A |
| 61 | B |
| 62 | B |
| 63 | C |
| 64 | A |
| 65 | C |
| 66 | C |
| 67 | B |
| 68 | B |
| 69 | D |
| 70 | A |
| 71 | B |
| 72 | B |
| 73 | D |
| 74 | D |
| 75 | A |
| 76 | A |
| 77 | B |
| 78 | B |
| 79 | C |
| 80 | C |
| 81 | B |
| 82 | C |
| 83 | C |
| 84 | B |
| 85 | B |
| 86 | A |
| 87 | D |
| 88 | D |
| 89 | D |
| 90 | D |
| 91 | D |
| 92 | C |
| 93 | D |
| 94 | D |
| 95 | C |
| 96 | C |
| 97 | D |
| 98 | D |
| 99 | D |
| 100 | D |
| 101 | D |
| 102 | D |
| 103 | D |
| 104 | D |
| 105 | D |
| 106 | D |
| 107 | D |
| 108 | D |
| 109 | B |
| 117 | A |
| 118 | A |
| 125 | B |
| 126 | D |
| 139 | D |
| 145 | B |
| 146 | C |
| 147 | B |
| 148 | B |
| 149 | B |
| 150 | A |
| 151 | A |
| 152 | B |
| 153 | A |
| 154 | A |
| 155 | D |
| 156 | A |
| 157 | D |
| 158 | D |
| 159 | C |
| 160 | B |
| 161 | C |
| 162 | C |
| 163 | B |
| 164 | D |
| 165 | B |
| 166 | C |
| 167 | D |
| 168 | D |
| 169 | C |
| 170 | D |
| 171 | C |
| 172 | D |
| 173 | D |
| 174 | D |
| 175 | D |
| 176 | D |
| 177 | C |
| 178 | D |
| 179 | C |
| 180 | A |
| 181 | D |
| 182 | D |
| 183 | B |
| 184 | A |
| 185 | A |

### Example 157. AKT1 Kinase Biological Activity Assay for Exemplary Compounds

AKT1 kinase assay: Inhibitory effects of compounds on wildtype AKT1 were undertaken in a 10-pt dose response kinase activity assay measuring phospho-peptide product formation based on TR-FRET signal as described in the following paragraph.

Purified human recombinant kinase full-length wildtype AKT1 was expressed in insect cells and activated in vitro by PDPK1. Ulight™—CREBtide (PerkinElmer, catalog number TRF0107) was used as the peptide substrate. The reaction buffer consisted of 50 mM HEPES pH 7.5, 10 mM MgCl₂, 0.01% Triton X-100, 0.01% BSA, 2 mM DTT, 0.6 nM AKT1, 50 nM Ulight™-CREBtide and either 50 μM ATP or 2 mM ATP. The compound solution was prepared as 10 mM DMSO stock. For the assay, a 1:3 serial dilution 10-pt dose response of test compounds, in duplicate, for each concentration were dispensed into a 384-well plate (Corning, catalog number 4513). Final DMSO concentration in the assay was 1%. The enzyme mixture (5 μL, 1.2 nM) were added and the mixture was incubated for 15 min at RT prior to the start of the kinase reaction by the addition of 5 μL substrate mixture (100 nM Ulight™ CREBtide). The reaction was stopped after 60 min of incubation at RT by the addition of 10 μL of Detection Mix (PerkinElmer, #CR97-100) consisting of Europium-anti-phospho-CREB (Ser133) (Perkin Elmer, catalog numberTRF0200). Incubation with the detection mix for a further 60 min allowed binding of antibody to the phospho-Ulight™ CREBtide. The fluorescence emissions at 620 nm and 665 nm after excitation at 350 om was measured using Envision reader. The ratio of the emissions at 665 nm and at 620 nm provided the measure for the amount of phosphorylated CREBtide product of AKT1 kinase activity. To determine the IC₅₀ values, the data was normalized (enzyme reaction without inhibitor=0% inhibition, all other assay components but no enzyme=100% inhibition) and IC₅₀ values were calculated by a 4-parameter fit using an in-house developed protocol.

Results are provided in Table 5. The symbol +++ represents an IC₅₀ value <500 nM; the symbol ++ represents an IC₅₀ value ≥500 nM.

TABLE 5

| Compound No. | AKT1 Lance IC₅₀ |
|---|---|
| 1 | +++ |
| 2 | ++ |
| 3 | +++ |
| 4 | +++ |
| 5 | +++ |
| 6 | +++ |
| 7 | +++ |
| 8 | +++ |
| 9 | +++ |
| 10 | +++ |
| 11 | +++ |
| 12 | +++ |
| 13 | ++ |
| 14 | +++ |
| 15 | +++ |
| 16 | +++ |
| 17 | +++ |
| 18 | +++ |
| 19 | +++ |
| 20 | +++ |
| 21 | +++ |
| 22 | +++ |
| 23 | +++ |
| 24 | +++ |
| 25 | +++ |
| 26 | +++ |
| 27 | +++ |
| 28 | ++ |
| 29 | +++ |
| 30 | +++ |
| 31 | +++ |
| 32 | +++ |
| 33 | +++ |
| 34 | +++ |
| 35 | +++ |
| 36 | +++ |
| 37 | +++ |
| 38 | ++ |
| 39 | +++ |
| 40 | +++ |
| 41 | +++ |
| 42 | +++ |
| 43 | +++ |
| 44 | +++ |
| 45 | +++ |
| 46 | +++ |
| 47 | +++ |
| 48 | ++ |
| 49 | ++ |
| 50 | ++ |
| 51 | +++ |
| 52 | ++ |
| 53 | +++ |
| 54 | +++ |
| 55 | +++ |
| 56 | +++ |
| 57 | +++ |
| 58 | +++ |
| 59 | +++ |
| 60 | +++ |
| 61 | +++ |
| 62 | +++ |
| 63 | +++ |
| 64 | +++ |
| 65 | +++ |
| 66 | ++ |
| 67 | +++ |
| 68 | +++ |
| 69 | +++ |
| 70 | +++ |
| 71 | +++ |
| 72 | +++ |
| 73 | +++ |
| 74 | ++ |
| 75 | +++ |
| 76 | +++ |
| 77 | +++ |
| 78 | +++ |
| 79 | +++ |
| 80 | +++ |
| 81 | +++ |
| 82 | +++ |
| 83 | +++ |
| 84 | +++ |
| 85 | +++ |
| 86 | +++ |
| 87 | +++ |
| 88 | +++ |
| 89 | +++ |
| 90 | +++ |
| 91 | +++ |
| 92 | +++ |
| 93 | +++ |
| 94 | +++ |
| 95 | +++ |
| 96 | +++ |
| 97 | ++ |
| 98 | +++ |
| 99 | +++ |
| 100 | ++ |
| 101 | ++ |
| 102 | +++ |
| 103 | ++ |
| 104 | ++ |
| 105 | +++ |
| 106 | ++ |
| 107 | ++ |
| 108 | ++ |
| 109 | +++ |
| 117 | +++ |
| 118 | +++ |
| 125 | +++ |
| 126 | +++ |
| 139 | +++ |
| 145 | +++ |
| 146 | +++ |
| 147 | +++ |
| 148 | +++ |
| 149 | +++ |
| 150 | +++ |
| 151 | +++ |
| 152 | +++ |
| 153 | +++ |
| 154 | +++ |
| 155 | +++ |
| 156 | +++ |
| 157 | +++ |
| 158 | +++ |
| 159 | +++ |
| 160 | +++ |
| 161 | +++ |
| 162 | +++ |
| 163 | +++ |
| 164 | +++ |
| 165 | +++ |
| 166 | +++ |
| 167 | +++ |
| 168 | +++ |
| 169 | +++ |
| 170 | +++ |
| 171 | +++ |
| 172 | +++ |
| 173 | +++ |
| 174 | ++ |
| 175 | ++ |
| 176 | ++ |
| 177 | +++ |
| 178 | +++ |
| 179 | +++ |
| 180 | +++ |
| 181 | ++ |
| 182 | +++ |
| 183 | +++ |
| 184 | +++ |
| 185 | +++ |

### INCORPORATION BY REFERENCE

The entire disclosure of each of the patent documents and scientific articles referred to herein is incorporated by reference for all purposes.

### EQUIVALENTS

The invention may be embodied in other specific forms without departing from the spirit or essential characteristics thereof. The foregoing embodiments are therefore to be considered in all respects illustrative rather than limiting the invention described herein. Scope of the invention is thus indicated by the appended claims rather than by the foregoing description, and all changes that come within the meaning and range of equivalency of the claims are intended to be embraced therein.

1. A compound represented by Formula I: 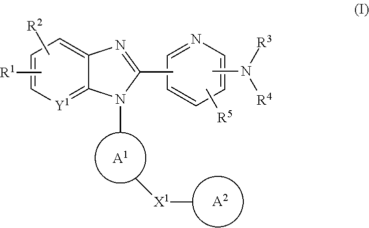
or a pharmaceutically acceptable salt thereof; wherein:
R¹ is phenyl, a 5-6 membered heteroaryl containing 1 or 2 heteroatoms selected from nitrogen, C₁₋₄ alkyl, C₁₋₄ haloalkyl, C₁₋₄ alkoxyl, C₁₋₄ haloalkoxy, cyano, C₃₋₄ cycloalkyl, —C(O)N(R¹⁰)(R¹¹), or hydrogen, wherein the phenyl and heteroaryl are each substituted with m occurrences of R¹³;
R², R³, R⁴, R⁵, and R⁹ are independently hydrogen or C₁₋₄ alkyl;
R⁶ is halo, C₁₋₄ haloalkyl, or C₁₋₄ alkyl;
R⁷ represents independently for each occurrence —(C₀₋₄ alkylene), —CO₂R⁹, hydroxyl, C₁₋₄ alkoxyl, C₁₋₄ alkyl, C₁₋₄ haloalkyl, cyano, —(C₀₋₄ alkylene)-C(O)N(R¹⁰)(R¹¹), —(C₀₋₄ alkylene)-N(R¹⁰)C(O)R¹², —SO₃R¹¹, —S(O)₂N(R¹⁰)(R¹¹), —S(O)₂R¹², or —N(R¹⁰)(R¹¹);
R⁸ represents independently for each occurrence hydroxyl, C₁₋₄ alkoxyl, C₁₋₄ alkyl, C₁₋₄ haloalkyl, halo, cyano, or —N(R¹⁰)(R¹¹);
R¹⁰ and R¹¹ each represent independently for each occurrence hydrogen or C₁₋₄ alkyl, or R¹⁰ and R¹¹ are taken together with the nitrogen atom to which they are attached to form a 3-7 membered ring;
R¹² represents independently for each occurrence C₁₋₆ alkyl or C₃₋₆ cycloalkyl,
R¹³ represents independently for each occurrence halo, C₁₋₄ alkyl, —(C₀₋₄ alkylene)-C(O)N(R¹⁰)(R¹¹), —(C₀₋₄ alkylene)-N(R¹⁰)C(O)R¹², —C(O)N(R¹⁰)-phenyl, or —N(R¹⁰)C(O)-phenyl;
A¹ is phenylene or pyridinylene, each of which is substituted with 0 or 1 occurrence of R⁶;
A² is phenyl or pyridinyl, each of which is substituted with (i) 1 occurrence of R⁷ and (ii) n occurrences of R⁸,
X¹ is —N(R⁹)C(O)-Ψ, —C(O)N(R⁹)-Ψ, —(C₁₋₃ alkylene)-N(R⁹)C(O)-Ψ, -(3-6 membered saturated heterocyclylene containing 1 or 2 heteroatoms selected from nitrogen)-C(O)-Ψ, —N(R⁹)C(R¹⁰)(R¹¹)-Ψ, —C(R⁹)(R¹⁰)N(R⁹)C(R⁹)(R¹⁰)—, —C(O)N(R⁹)—(C₁₋₃ alkylene)-Ψ, or -(3-6 membered saturated heterocyclylene containing 1 or 2 heteroatoms selected from nitrogen)-C(R¹⁰)(R¹¹)-Ψ; wherein Ψ is a bond to A²;
Y¹ is N or —C(H)—; and
m and n are independently 0, 1, or 2.

2. The compound of claim 1, wherein the compound is represented by Formula I: 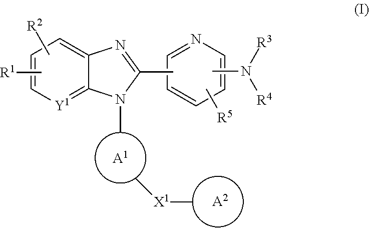
or a pharmaceutically acceptable salt thereof; wherein:
R¹ is phenyl, a 5-6 membered heteroaryl containing 1 or 2 heteroatoms selected from nitrogen, C₁₋₄ haloalkyl, C₁₋₄ alkoxyl, cyano, C₃₋₄ cycloalkyl, —C(O)N(R¹⁰)(R¹¹), or hydrogen, wherein the phenyl and heteroaryl are each substituted with m occurrences of R¹³;
R², R³, R⁴, R⁵, and R⁹ are independently hydrogen or C₁₋₄ alkyl;
R⁶ is halo, C₁₋₄ haloalkyl, or C₁₋₄ alkyl;
R⁷ represents independently for each occurrence —(C₀₋₄ alkylene)-CO₂R⁹, hydroxyl, C₁₋₄ alkoxyl, C₁₋₄ alkyl, C₁₋₄ haloalkyl, cyano, —(C₀₋₄ alkylene)-C(O)N(R¹⁰)(R¹¹), —(C₀₋₄ alkylene)-N(R¹⁰)C(O)R¹², —SO₃R¹¹, —S(O)₂N(R¹⁰)(R¹¹), —S(O)₂R¹², or —N(R¹⁰)(R¹¹);
R⁸ represents independently for each occurrence hydroxyl, C₁₋₄ alkoxyl, C₁₋₄ alkyl, C₁₋₄ haloalkyl, halo, cyano, or —N(R¹⁰)(R¹¹);
R¹⁰ and R¹¹ each represent independently for each occurrence hydrogen or C₁₋₄ alkyl, or R¹⁰ and R¹¹ are taken together with the nitrogen atom to which they are attached to form a 3-7 membered ring;
R¹² represents independently for each occurrence C₁₋₆ alkyl or C₃₋₆ cycloalkyl;
R¹³ represents independently for each occurrence halo, C₁₋₄ alkyl, —(C₀₋₄ alkylene)-C(O)N(R¹⁰)(R¹¹), —(C₀₋₄ alkylene)-N(R¹⁰)C(O)R¹², —C(O)N(R¹⁰)-phenyl, or —N(R¹⁰)C(O)-phenyl;
A¹ is phenylene or pyridinylene, each of which is substituted with 0 or 1 occurrence of R⁶;
A² is phenyl or pyridinyl, each of which is substituted with (i) 1 occurrence of R⁷ and (ii) n occurrences of R⁸;
X¹ is —N(R⁹)C(O)-Ψ, —(C₁₋₃ alkylene)-N(R⁹)C(O)-Ψ, -(3-6 membered saturated heterocyclylene containing 1 or 2 heteroatoms selected from nitrogen)-C(O)—, —N(R⁹)C(R¹⁰)(R¹¹)-Ψ, —C(R⁹)(R¹⁰)N(R⁹)C(R⁹)(R¹⁰)—, —C(O)N(R⁹)—(C₁₋₃ alkylene)-Ψ, or -(3-6 membered saturated heterocyclylene containing 1 or 2 heteroatoms selected from nitrogen)-C(R¹⁰)(R¹¹)-Ψ; wherein Ψ is a bond to A²;
Y¹ is N or —C(H)—; and
m and n are independently 0, 1, or 2

3. The compound of claim 1 or 2, wherein the compound is a compound of Formula I.

4. The compound of claim 1 or 2, wherein the compound is represented by Formula Ia or a pharmaceutically acceptable salt thereof.

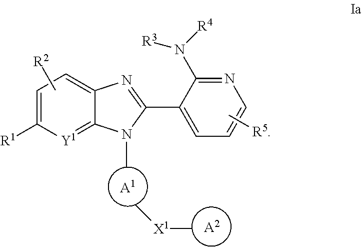

5. The compound of any one of claims 1-4, wherein Y¹ is N.

6. The compound of any one of claims 1-4, wherein Y¹ is —C(H)—.

7. The compound of any one of claims 1-6, wherein R¹ is phenyl.

8. The compound of any one of claims 1-6, wherein R¹ is C₁₋₄ haloalkyl, C₁₋₄ alkoxyl, or hydrogen.

9. The compound of any one of claims 1-8, wherein R² is hydrogen.

10. The compound of any one of claims 1-9, wherein R³ is hydrogen.

11. The compound of any one of claims 1-10, wherein R⁴ is hydrogen.

12. The compound of any one of claims 1-11, wherein R⁵ is hydrogen.

13. The compound of any one of claims 1-12, wherein X¹ is —N(R⁹)C(O)-Ψ.

14. The compound of any one of claims 1-12, wherein X¹ is —C(O)N(R⁹)-Ψ.

15. The compound of any one of claims 1-12, wherein X¹ is —(C₁₋₃ alkylene)-N(R⁹)C(O)-Ψ.

16. The compound of any one of claims 1-12, wherein X¹ is —(CH₂)—N(R⁹)C(O)-Ψ.

17. The compound of any one of claims 1-12, wherein X¹ is a -(3-6 membered saturated heterocyclylene containing 1 or 2 heteroatoms selected from nitrogen)-C(O)-Ψ.

18. The compound of any one of claims 1-12, wherein X¹ is a -(4-membered saturated heterocyclylene containing 1 heteroatom selected from nitrogen)-C(O)-Ψ.

19. The compound of any one of claims 1-12, wherein X¹ is

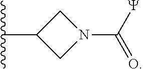

20. The compound of any one of claims 1-19, wherein R⁹ is hydrogen.

21. The compound of any one of claims 1-20, wherein A¹ is phenylene substituted with 0 or 1 occurrence of R⁶.

22. The compound of any one of claims 1-20, wherein A¹ is phenylene.

23. The compound of any one of claims 1-20, wherein A¹ is pyridinylene.

24. The compound of any one of claims 1-23, wherein A² is phenyl substituted with (i) 1 occurrence of R⁷ and (ii) n occurrences of R⁸.

25. The compound of any one of claims 1-23, wherein A² is pyridinyl substituted with (i) 1 occurrence of R⁷ and (ii) n occurrences of R⁸.

26. The compound of any one of claims 1-23, wherein A² is one of the following

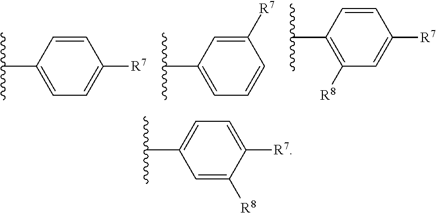

27. The compound of any one of claims 1-25, wherein n is 1.

28. The compound of any one of claims 1-25, wherein n is 0.

29. The compound of claim 1 or 2, wherein the compound is a compound of Formula Ib, Ic, Id, Ie, If, or Ig, or a pharmaceutically acceptable salt thereof:

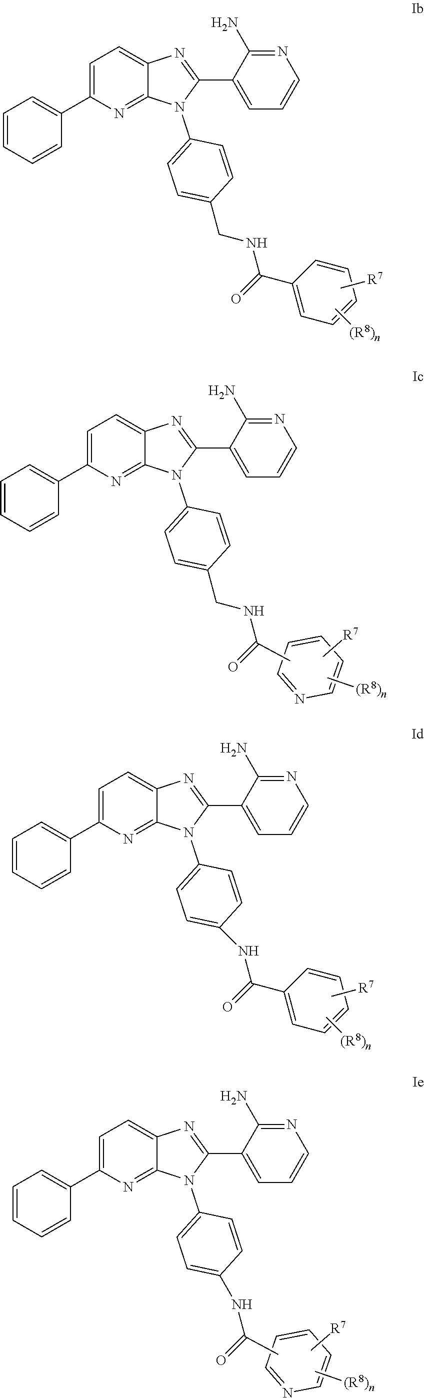

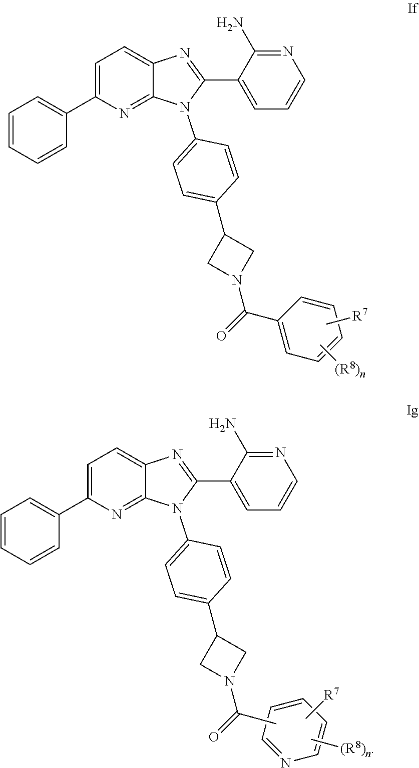

30. The compound of claim 1 or 2, wherein the compound is a compound of Formula Ih, Ii, Ij, Ik, Il, or Im, or a pharmaceutically acceptable salt thereof:

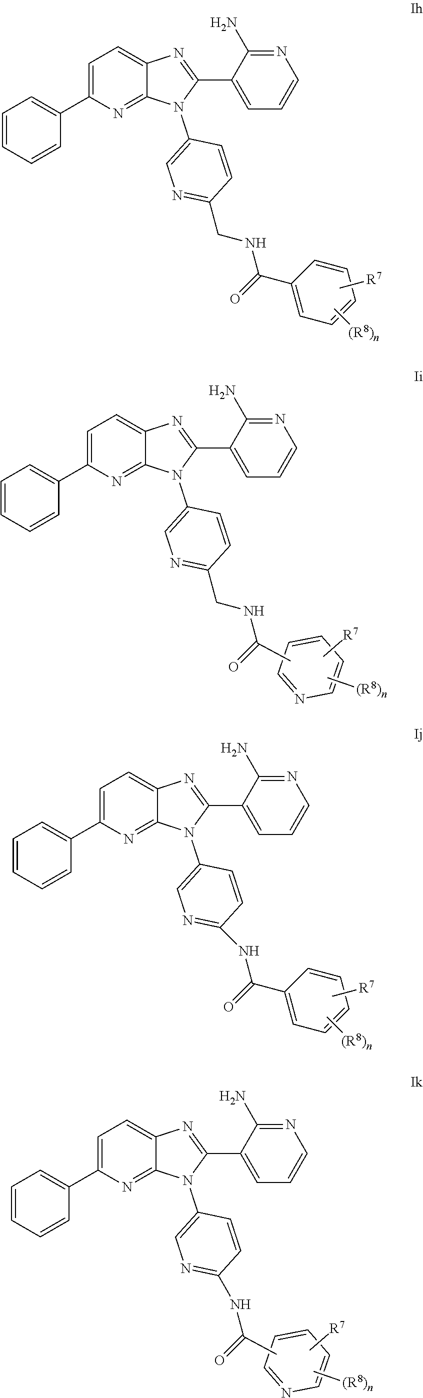

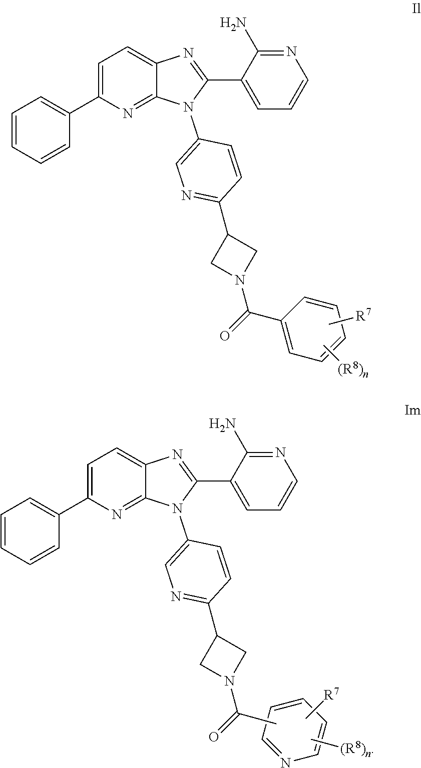

31. The compound of any one of claims 1-27, 29, or 30, wherein R⁸ represents independently for each occurrence hydroxyl, C₁₋₄ alkoxyl, and C₁₋₄ alkyl.

32. The compound of any one of claims 1-27, 29, or 30, wherein R⁸ is hydroxyl.

33. The compound of any one of claims 1-27, 29, or 30, wherein R⁸ represents independently for each occurrence C₁₋₄ alkyl.

34. The compound of any one of claims 1-27, 29, or 30, wherein R⁸ is methyl.

35. The compound of any one of claims 1-27, 29, or 30, wherein R⁸ represents independently for each occurrence C₁₋₄ alkoxyl.

36. The compound of any one of claims 1-27, 29, or 30, wherein R⁸ is methoxy.

37. The compound of any one of claims 1-36, wherein R⁷ is —(C₀₋₄ alkylene)-CO₂R⁹.

38. The compound of any one of claims 1-36, wherein R⁷ is —CO₂H.

39. The compound of any one of claims 1-36, wherein R⁷ is —CH₂CO₂H.

40. The compound of any one of claims 1-36, wherein R⁷ is —CH₂CH₂CO₂H.

41. The compound of any one of claims 1-36, wherein R⁷ is —C(H)(CH₃) CO₂H.

42. The compound of any one of claims 1-36, wherein R⁷ is hydroxyl.

43. The compound of any one of claims 1-36, wherein R⁷ is C₁₋₄ alkoxyl.

44. The compound of any one of claims 1-36, wherein R⁷ is cyano.

45. The compound of any one of claims 1-36, wherein R⁷ is —(C₀₋₄ alkylene)-C(O)N(R¹⁰)(R¹¹), —(C₀₋₄ alkylene)-N(R¹⁰)C(O)R¹², or —N(R¹⁰) (R¹¹).

46. The compound of any one of claims 1-36, wherein R⁷ is-SO₃R¹¹, —S(O)₂N(R¹⁰)(R¹¹), or —S(O)₂R¹².

47. A compound in Table 1 herein, or a pharmaceutically acceptable salt thereof.

48. A pharmaceutical composition comprising a compound of any one of claims 1-47 and a pharmaceutically acceptable carrier.

49. A method of treating a disease or disorder associated with aberrant AKT1 signaling, comprising administering to a subject in need thereof a therapeutically effective amount of a compound of any one of claims 1-47 to treat the disease or disorder.

50. The method of claim 49, wherein the disease or disorder associated with aberrant AKT1 signaling is an AKT1 E17K associated disease or disorder.

51. A method of treating cancer, comprising administering to a subject in need thereof a therapeutically effective amount of a compound of any one of claims 1-47 to treat the cancer.

52. The method of claim 51, wherein the cancer is a solid tumor.

53. The method of claim 51, wherein the cancer is ovarian cancer, uterine cancer, endometrial cancer, cervical cancer, prostate cancer, testicular cancer, breast cancer, brain cancer, lung cancer, oral cancer, esophageal cancer, head and neck cancer, stomach cancer, colon cancer, rectal cancer, skin cancer, sebaceous gland carcinoma, bile duct cancer, gallbladder cancer, liver cancer, pancreatic cancer, bladder cancer, urinary tract cancer, kidney cancer, eye cancer, thyroid cancer, lymphoma, leukemia, urothelial cancer, colorectal cancer, or glioblastoma multiforme.

54. The method of claim 51, wherein the cancer is a breast invasive carcinoma, colon adenocarcinoma, head and neck cancer, lung adenocarcinoma, rectal adenocarcinoma, acute myeloid leukemia, glioblastoma multiforme, brain lower grade glioma, colorectal cancer, uterine corpus endometrial carcinoma, cervical cancer, endocervical cancer, thyroid carcinoma, prostate adenocarcinoma, skin cutaneous melanoma, bladder urothelial carcinoma, head and neck squamous cell carcinoma, or stomach adenocarcinoma.

55. The method of claim 51, wherein the cancer is an adenocarcinoma, squamous cell carcinoma, epithelial neoplasm, glioma, ductal neoplasm, lobular neoplasm, cystic neoplasm, mucinous neoplasm, or serous neoplasm, acinar cell neoplasm, basal cell neoplasm, fibroepithelial neoplasm, transitional cell papilloma, or transitional cell carcinoma.

56. The method of claim 51, wherein the cancer is a cervical cancer, uterine cancer, breast cancer, thyroid cancer, prostate cancer, lung cancer, bladder cancer, skin cancer, stomach cancer, lymphoma, or leukemia.

57. The method of claim 51, wherein the cancer is a lymphoma or leukemia.

58. The method of any one of claims 51-57, wherein the cancer has an AKT1 mutation.

59. The method of any one of claims 51-57, wherein the cancer has an AKT1 E17K mutation.

60. A method of treating a disease or disorder associated with active PI3K signaling, comprising administering to a subject in need thereof a therapeutically effective amount of a compound of any one of claims 1-47 to treat the disease or disorder.

61. The method of any one of claims 49-60, wherein the subject is a human.

62. A method of inhibiting AKT1 activity, comprising contacting an AKT1 with an effective amount of a compound of any one of claims 1-47 to thereby inhibit the AKT1 activity.

63. The method of claim 62, wherein the AKT1 is AKT1 E17K.
